(19) United States
(12) Patent Application Publication
(10) Pub. No.: US 2012/0053148 A1
(43) Pub. Date: Mar. 1, 2012

(54) INHIBITORS OF HEPATITIS C VIRUS

(75) Inventors: Zhenhong R. Cai, Palo Alto, CA (US); Zhimin Du, Foster City, CA (US); Mingzhe Ji, Union City, CA (US); Haolun Jin, Foster City, CA (US); Choung U. Kim, San Carlos, CA (US); Michael R. Mish, La Honda, CA (US); Barton W. Phillips, San Mateo, CA (US); Hyung-jung Pyun, Fremont, CA (US); Xiaoning C. Sheng, San Carlos, CA (US); Qiaoyin Wu, Foster City, CA (US); Catalin Sebastian Zonte, San Francisco, CA (US)

(73) Assignee: GILEAD SCIENCES, INC., Foster City, CA (US)

(21) Appl. No.: 13/157,127

(22) Filed: Jun. 9, 2011

Related U.S. Application Data
(60) Provisional application No. 61/353,113, filed on Jun. 9, 2010.

(51) Int. Cl. A61K 31/675; C07D 417/12; A61P 31/14; A61K 31/541; C07D 401/14; C07D 401/12; A61K 31/4709

(52) U.S. Cl. 514/82; 546/169; 544/58.2; 514/314; 514/228.2; 546/167

(57) ABSTRACT

The present application includes novel inhibitors of HCV, compositions containing such compounds, therapeutic methods that include the administration of such compounds.

### CROSS-REFERENCE TO RELATED APPLICATIONS

Priority is claimed to U.S. Provisional Application No. 61/353,113, filed 9 Jun. 2010, herein incorporated by reference in its entirety for all purposes.

### FIELD OF THE INVENTION

The present application includes novel inhibitors of HCV, compositions containing such compounds, therapeutic methods that include the administration of such compounds.

### BACKGROUND OF THE INVENTION

Hepatitis is a disease occurring throughout the world. Hepatitis is generally of viral nature, although there are other known causes. Viral hepatitis is by far the most common form of hepatitis. In the U.S. nearly 750,000 are affected by hepatitis each year, and out of those, more than 150,000 are infected with the hepatitis C virus (“HCV”). HCV is a positive-stranded RNA virus belonging to the Flaviviridae family and has closest relationship to the pestiviruses that include hog cholera virus and bovine viral diarrhea virus (BVDV).

HCV is believed to replicate through the production of a complementary negative-strand RNA template. The HCV genome is a single-stranded, positive-sense RNA of about 9,600 bp coding for a polyprotein of 3009-3030 amino-acids, which is cleaved co- and post-translationally by cellular and two viral proteinases into mature viral proteins (core, E1, E2, p7, NS2, NS3, NS4A, NS4B, NS5A, NS5B). The structural proteins, E1 and E2, are believed to be embedded into a viral lipid envelope and form stable heterodimers. The structural core protein is believed to interact with the viral RNA genome to form the nucleocapsid. The nonstructural proteins designated NS2 to NS5 include proteins with enzymatic functions involved in virus replication and protein processing including a polymerase, protease, and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post-transfusion has been markedly reduced lately due to advances in diagnostic tools used to screen blood donors.

One available treatment for HCV infection is interferon-α (IFN-α). According to different clinical studies, however, only 70% of treated patients normalize alanine aminotransferase (ALT) levels in the serum and after discontinuation of IFN, 35% to 45% of these responders relapse. In general, only 20% to 25% of patients have long-term responses to IFN. Clinical studies have shown that combination treatment with IFN and ribavirin (RIBA) results in a superior clinical response than IFN alone. Different genotypes of HCV respond differently to IFN therapy; genotype 1 is more resistant to IFN therapy than types 2 and 3.

There is therefore a great need for the development of anti-viral agents.

### SUMMARY OF THE INVENTION

One embodiment of the present invention includes compounds of Formula 1:

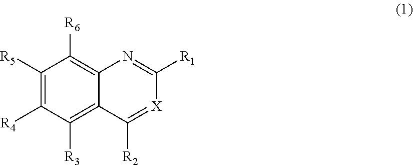

or a pharmaceutically acceptable salt, solvate, tautomer, or prodrug thereof, wherein:
R₁ is selected from the group consisting of optionally substituted C₁-C₆ alkyl, optionally substituted C₁-C₆ alkenyl, optionally substituted aryl, optionally substituted heteroaryl, optionally substituted arylalkyl, optionally substituted heteroarylalkyl, optionally substituted arylalkenyl, —C(O)NZ₁R₇, —NHR₈, —YR₉, —NHYR₉, —C(O)NR₁₀R₁₁, —C═N—NR_{A}R_{B}, and —C═N—O;
wherein Z₁ is hydrogen or C₁-C₆ alkyl;
wherein R₇ and R₈ are independently selected from the group consisting of H, sulfonyl, optionally substituted heterocyclylalkyl, optionally substituted aryl, optionally substituted arylalkyl, optionally substituted heteroaryl, and optionally substituted heteroarylalkyl;
wherein R₉ is selected from the group consisting of optionally substituted arylalkyl and optionally substituted heteroarylalkyl;
wherein R₁₀ and R₁₁, together with the nitrogen to which they are both attached, form a heterocycle which is optionally substituted;
wherein Y is selected from the group consisting of C(O) and SO₂; and
wherein R_{A} and R_{B} are each independently C₁-C₆ alkyl;
R₂ is selected from the group consisting of hydrogen, hydroxyl, cyano, sulfonamide, sulfone, optionally substituted C₁-C₆ alkyl, optionally substituted C₁-C₆ alkoxy, optionally substituted aryl, optionally substituted heteroaryl, optionally substituted cycloalkyl, optionally substituted heterocycle, NHR₁₂, NR₁₃R₁₄, S(O)₀₋₂R₁₅, and halogen provided that when X is N then R₂ is not halogen;
wherein R₁₂, R₁₃ and R₁₄ are each independently selected from the group consisting of hydrogen, sulfonyl, sulfone, sulfonamide, acetyl, optionally substituted C₁-C₆ alkyl, optionally substituted C₁-C₆ alkenyl, optionally substituted C₁-C₆ alkoxy, and hydroxy; and
wherein R₁₅ is selected from the group consisting of sulfonyl, sulfone, sulfonamide, acetyl, optionally substituted C₁-C₆ alkyl, optionally substituted C₁-C₆ alkenyl, optionally substituted C₁-C₆ alkoxy, and hydroxy;
R₃ is selected from the group consisting of hydrogen, halogen, optionally substituted C₁-C₆ alkyl, optionally substituted C₁-C₆ alkenyl, C₁-C₆ haloalkyl, cyano, optionally substituted C₁-C₆ alkoxy, sulfone, and sulfonamide;
R₄ is selected from the group consisting of optionally substituted aryl, and optionally substituted heteroaryl, provided that said heteroaryl is not a bicyclic heteroaryl (e.g., said heteroaryl can be a monocyclic heteroaryl);
R₅ is selected from the group consisting of hydrogen, halogen, optionally substituted C₁-C₆ alkyl, optionally substituted C₁-C₆ alkenyl, C₁-C₆ haloalkyl, cyano, optionally substituted C₁-C₆ alkoxy, sulfone, and sulfonamide, provided that R₅ is not silicon and does not include a chemical group comprising a silicon atom;
R₆ is selected from the group consisting of hydrogen, halogen, haloalkyl, C₁-C₆ haloalkoxy, optionally substituted C₁-C₆ alkyl, optionally substituted C₂-C₆ alkenyl, optionally substituted C₂-C₆ alkynyl, optionally substituted aryl, optionally substituted arylalkyl, optionally substituted arylalkenyl, optionally substituted heteroaryl, optionally substituted heteroarylalkyl, optionally substituted heteroarylalkenyl, and —C(O)OH;
X is selected from the group consisting of N and CH;
and provided that Formula 1 does not include a compound selected from the group consisting of

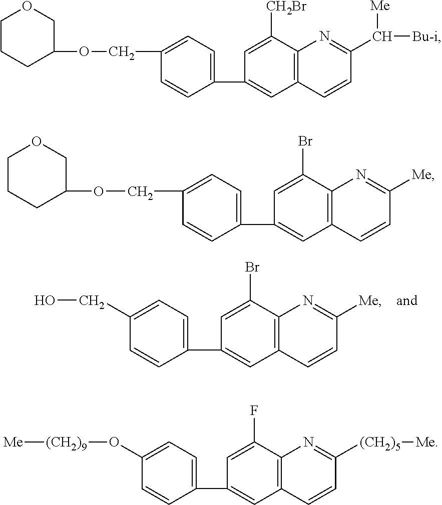

In another embodiment, the present invention provides compounds of Formula 2:

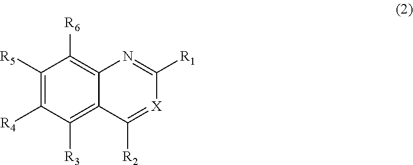

or a pharmaceutically acceptable salt, solvate, tautomer, or prodrug thereof, wherein:
R₁ is selected from the group consisting of optionally substituted C₁-C₆ alkyl, optionally substituted C₁-C₆ alkenyl, optionally substituted aryl, optionally substituted heteroaryl, optionally substituted arylalkyl, optionally substituted heteroarylalkyl, optionally substituted arylalkenyl, —C(O)NZ₁R₇, —NHR₈, —YR₉, —NHYR₉, —C(O)NR₁₀R₁₁, —C═N—NR_{A}R_{B}, and —C═N—O;
wherein Z₁ is hydrogen or C₁-C₆ alkyl;
wherein R₇ and R₈ are independently selected from the group consisting of H, sulfonyl, optionally substituted heterocyclylalkyl, optionally substituted heterocyclylalkylalkyl, optionally substituted aryl, optionally substituted arylalkyl, optionally substituted heteroaryl, optionally substituted heteroarylalkyl, optionally substituted heterocyclylalkynyl, optionally substituted heterocyclylalkenyl, optionally substituted arylalkenyl, optionally substituted arylalkynyl, optionally substituted cycloalkyl, optionally substituted cycloalkylalkyl, optionally substituted alkyl, and hydrogen;
wherein R₉ is selected from the group consisting of optionally substituted arylalkyl and optionally substituted heteroarylalkyl;
wherein R₁₀ and R₁₁, together with the nitrogen to which they are both attached, form a heterocycle which is optionally substituted;
wherein Y is selected from the group consisting of C(O) and SO₂; and
wherein R_{A} and R_{B} are each independently C₁-C₆ alkyl;
R₂ is selected from the group consisting of hydrogen, hydroxyl, cyano, sulfonamide, sulfone, optionally substituted C₁-C₆ alkyl, optionally substituted C₂-C₆ alkene, optionally substituted C₂-C₆ alkyne, optionally substituted C₁-C₆ alkoxy, optionally substituted aryl, optionally substituted heteroaryl, optionally substituted cycloalkyl, optionally substituted heterocycle, NHR₁₂, NR₁₃R₁₄, S(O)₀₋₂R₁₅, and halogen provided that when X is N then R₂ is not halogen;

wherein R₁₂, R₁₃ and R₁₄ are each independently selected from the group consisting of hydrogen, sulfonyl, sulfone, sulfonamide, acetyl, optionally substituted C₁-C₆ alkyl, optionally substituted C₁-C₆ alkenyl, optionally substituted C₁-C₆ alkoxy, and hydroxy; and

wherein R₁₅ is selected from the group consisting of sulfonyl, sulfone, sulfonamide, acetyl, optionally substituted C₁-C₆ alkyl, optionally substituted C₁-C₆ alkenyl, optionally substituted C₁-C₆ alkoxy, and hydroxy;
R₃ is selected from the group consisting of hydrogen, halogen, optionally substituted C₁-C₆ alkyl, optionally substituted C₂-C₆ alkenyl, C₁-C₆ haloalkyl, cyano, optionally substituted C₁-C₆ alkoxy, sulfone, and sulfonamide;
R₄ is selected from the group consisting of optionally substituted aryl, optionally substituted heteroaryl, provided that said heteroaryl is not a bicyclic heteroaryl, —C(O)NR₁₆R₁₇, optionally substituted cycloalkyl, optionally substituted cycloalkoxy, optionally substituted C₁-C₆ alkoxy, —NR₁₈R₁₉, optionally substituted C₁-C₆ alkyl, optionally substituted C₂-C₆ alkenyl, optionally substituted C₂-C₆ alkynyl, cyano, —C(O)R₂₀R₂₁, —NR₂₂C(O)R₂₃, —NR₂₂S(O)₂R₂₃, optionally substituted heterocycloalkyl;
wherein each of R₁₆, R₁₇, R₁₈, R₁₉, R₂₀, R₂₁, R₂₂ and R₂₃ are independently selected from the group consisting of hydrogen, optionally substituted C₁-C₆ alkyl, and optionally substituted C₂-C₆ alkenyl;
R₅ is selected from the group consisting of hydrogen, halogen, optionally substituted C₁-C₆ alkyl, optionally substituted C₂-C₆ alkenyl, C₁-C₆ haloalkyl, cyano, optionally substituted C₁-C₆ alkoxy, sulfone, and sulfonamide, provided that R₅ does not include a chemical group comprising a silicon atom;
R₆ is selected from the group consisting of hydrogen, halogen, haloalkyl, C₁-C₆ haloalkoxy, optionally substituted C₁-C₆ alkyl, optionally substituted C₂-C₆ alkenyl, optionally substituted C₂-C₆ alkynyl, optionally substituted aryl, optionally substituted arylalkyl, optionally substituted arylalkenyl, optionally substituted heteroaryl, optionally substituted heteroarylalkyl, optionally substituted heteroarylalkenyl, and —C(O)OH;
X is selected from the group consisting of N and CH;
and provided that Formula 2 does not include a compound selected from the group consisting of

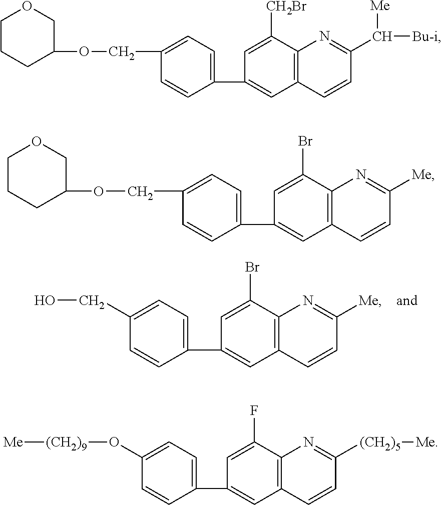

In another embodiment, there is provided a compound of Formula (3),

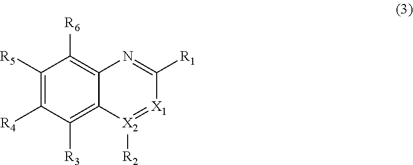

or a pharmaceutically acceptable salt, solvate, tautomer, or prodrug thereof, wherein:
X₁ is CR₆ or N;
X₂ is C or N
R₁ is C(O)NZ₁R₇ or heteroaryl, wherein said heteroaryl is optionally substituted with NZ₁R₇;
R₂ is:
   a) not present when X₂ is N, or
   b) H, (C₁-C₃)alkyl, (C₂-C₆)alkenyl, (C₂-C₆)alkynyl, (C₁-C₃)alkoxy, hydroxyl, halo, amino, amido, amino(C₁-C₈)alkylamido, heterocyclyl, sulfonyl, aminosulfonyl, amino(C₁-C₈)alkysulfonyl, cyano, or (C₁-C₃)haloalkyl;
R₃ is H, halo, (C₁-C₃)alkyl, or (C₁-C₃)haloalkyl;
R₄ is Halo, (C₁-C₆)alkyl, (C₂-C₆)alkenyl, (C₂-C₆)alkynyl, (C₁-C₆)alkoxy, cycloalkyl, heterocyclyl, aryl, heteroaryl, cycloalkoxy, aryloxy, amino, aminosulfonyl, alkylsulfonyl and amido, and wherein any of the preceding substitutents are optionally independently substituted with halo, amino, amido, (C₁-C₆)alkyl, (C₂-C₆)alkenyl, (C₂-C₆)alkynyl, (C₁-C₆)alkoxy, cycloalkyl, heterocyclyl, hydroxyl, and combinations thereof;
R₅ is H or halo;
R₆ is Halo, (C₁-C₈)alkyl, (C₂-C₈)alkenyl, C₂-C₈)alkynyl, (C₁-C₈)haloalkyl, (C₂-C₈)haloalkenyl, (C₂-C₈)haloalkynyl, (C₁-C₈)alkoxy, (C₁-C₈)haloalkoxy, heterocyclyl, heteroaryl, CO, C(O)OH, hydroxyl, (C₁-C₄)alkylsulfonyl, aminosulfonyl, amino(C₁-C₄)alkylsulfonyl or aryl;
R₇ is H, sulfonyl, (C₁-C₆)alkyl, (Cₜ—C₆)alkoxy, (C₂-C₆)alkenyl, (C₂-C₆)alkynyl, (C₁-C₆)alcohol, (C₁-C₆)alkylcycloalkyl, cyano(C₁-C₆)alkyl, (C₁-C₆)alkylcarbonyl, aryl, (C₁-C₆)arylalkyl, (C₁-C₆)alkoxyaryl (C₂-C₆)alkenylaryl (C₁-C₆)alkylheterocycle, (C₁-C₆)alkylheteroaryl,
wherein any of said (C₁-C₆)alkyl, (C₁-C₆)alkoxy, (C₂-C₆)alkenyl, (C₂-C₆)alkynyl,

(C₁-C₆)alcohol, (C₁-C₆)alkylcycloalkyl, (C₁-C₆)alkylnitrile, (C₁-C₆)alkylcarbonyl, aryl, (C₁-C₆)arylalkyl, (C₁-C₆)alkoxyaryl (C₂-C₆)alkenylaryl (C₁-C₆)alkylheterocycle, (C₁-C₆)alkylheteroaryl, are optionally substituted with carbonyl, (C₁-C₄)alkyl, (C₁-C₄)haloalkyl, hydroxyl, C(O)O—R₂₉; or,
Z₁ and R₇, together with the nitrogen atom to which they are attached, form a 5 or 6 membered heterocycle, said heterocycle optionally substituted with aryl or heteroaryl; and
R₂₉ is H or (C₁-C₄)alkyl.

In some embodiments, X₂ is N, and R₂ is not present.

In other embodiments, X₂ is C.

In another embodiment, there is provided a compound of Formula (4):

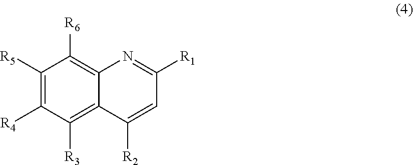

or a pharmaceutically acceptable salt, solvate, tautomer, or prodrug thereof, wherein:
R₁ is substituted C₁-C₆ alkyl, optionally substituted C₁-C₆ alkenyl, optionally substituted aryl, optionally substituted heteroaryl, optionally substituted arylalkyl, optionally substituted heteroarylalkyl, optionally substituted arylalkenyl, —C(O)NZ₁R₇, —NHR₈, —YR₉, —NHYR₉, —C(O)NR₁₀R₁₁, —C═N—NR_{A}R_{B}, or —C═N—O;
wherein Z₁ is H or C₁-C₆ alkyl;
wherein R₇ and R₈ are independently optionally substituted heterocyclylalkyl, optionally substituted heterocyclylalkylalkyl, optionally substituted aryl, optionally substituted arylalkyl, optionally substituted heteroaryl, optionally substituted heteroarylalkyl, optionally substituted heterocyclylalkynyl, optionally substituted heterocyclylalkenyl, optionally substituted arylalkenyl, optionally substituted arylalkynyl, optionally substituted cycloalkyl, optionally substituted cycloalkylalkyl, optionally substituted alkyl, or H;
wherein R₉ is optionally substituted arylalkyl or optionally substituted heteroarylalkyl;
wherein R₁₀ and R₁₁, together with the nitrogen to which they are both attached, form a heterocycle which is optionally substituted;
wherein Y is C(O) or SO₂; and
wherein R_{A} and R_{B} are each independently C₁-C₆ alkyl;
R₂ is H, hydroxyl, cyano, sulfonamide, sulfone, optionally substituted C₁-C₆ alkyl, optionally substituted C₂-C₆ alkene, optionally substituted C₂-C₆ alkyne, optionally substituted C₁-C₆ alkoxy, optionally substituted aryl, optionally substituted heteroaryl, optionally substituted cycloalkyl, optionally substituted heterocycle, NHR₁₂, NR₁₃R₁₄, S(O)₀₋₂R₁₅, or halogen;
wherein R₁₂, R₁₃ and R₁₄ are each independently H, sulfonyl, sulfone, sulfonamide, acetyl, optionally substituted C₁-C₆ alkyl, optionally substituted C₁-C₆ alkenyl, optionally substituted C₁-C₆ alkoxy, or hydroxy; and
wherein R₁₅ is sulfonyl, sulfone, sulfonamide, acetyl, optionally substituted C₁-C₆ alkyl, optionally substituted C₁-C₆ alkenyl, optionally substituted C₁-C₆ alkoxy, or hydroxy;
R₃ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide;
R₄ is optionally substituted aryl, optionally substituted heteroaryl, provided that said heteroaryl is not a bicyclic heteroaryl, —C(O)NR₁₆R₁₇, optionally substituted cycloalkyl, optionally substituted cycloalkoxy, optionally substituted C₁-C₆ alkoxy, —NR₁₈R₁₉, optionally substituted C₁-C₆ alkyl, optionally substituted C₂-C₆ alkenyl, optionally substituted C₂-C₆ alkynyl, cyano, —C(O)R₂OR₂₁, —NR₂₂C(O)R₂₃, —NR₂₂S(O)₂R₂₃, or optionally substituted heterocycloalkyl;
wherein each of R₁₆, R₁₇, R₁₈, R₁₉, R₂₀, R₂₁, R₂₂ and R₂₃ are independently H, optionally substituted C₁-C₆ alkyl, or optionally substituted C₂-C₆ alkenyl;
R₅ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide, provided that R₅ does not include a chemical group comprising a silicon atom;
R₆ is H, halogen, haloalkyl, C₁-C₆ haloalkoxy, optionally substituted C₁-C₆ alkyl, optionally substituted C₂-C₆ alkenyl, optionally substituted C₂-C₆ alkynyl, optionally substituted aryl, optionally substituted arylalkyl, optionally substituted arylalkenyl, optionally substituted heteroaryl, optionally substituted heteroarylalkyl, optionally substituted heteroarylalkenyl, or —C(O)OH.

In another embodiment, there is provided a compound of Formula 5:

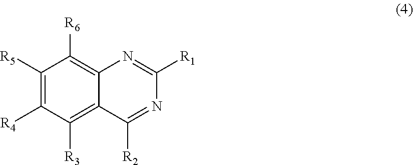

or a pharmaceutically acceptable salt, solvate, tautomer, or prodrug thereof, wherein:
R₁ is substituted C₁-C₆ alkyl, optionally substituted C₁-C₆ alkenyl, optionally substituted aryl, optionally substituted heteroaryl, optionally substituted arylalkyl, optionally substituted heteroarylalkyl, optionally substituted arylalkenyl, —C(O)NZ₁R₇, —NHR₈, —YR₉, —NHYR₉, —C(O)NR₁₀R₁₁, —C═N—NR_{A}R_{B}, or —C═N—O;
wherein Z₁ is H or C₁-C₆ alkyl;
wherein R₇ and R₉ are independently optionally substituted heterocyclylalkyl, optionally substituted heterocyclylalkylalkyl, optionally substituted aryl, optionally substituted arylalkyl, optionally substituted heteroaryl, optionally substituted heteroarylalkyl, optionally substituted heterocyclylalkynyl, optionally substituted heterocyclylalkenyl, optionally substituted arylalkenyl, optionally substituted arylalkynyl, optionally substituted cycloalkyl, optionally substituted cycloalkylalkyl, optionally substituted alkyl, or H;
wherein R₉ is optionally substituted arylalkyl or optionally substituted heteroarylalkyl;
wherein R₁₀ and R₁₁, together with the nitrogen to which they are both attached, form a heterocycle which is optionally substituted;
wherein Y is C(O) or SO₂; and
wherein R_{A} and R_{B} are each independently C₁-C₆ alkyl;
R₂ is H, hydroxyl, cyano, sulfonamide, sulfone, optionally substituted C₁-C₆ alkyl, optionally substituted C₂-C₆ alkene, optionally substituted C₂-C₆ alkyne, optionally substituted C₁-C₆ alkoxy, optionally substituted aryl, optionally substituted heteroaryl, optionally substituted cycloalkyl, optionally substituted heterocycle, NHR₁₂, NR₁₃R₁₄, S(O)₀₋₂R₁₅, or halogen;
wherein R₁₂, R₁₃ and R₁₄ are each independently H, sulfonyl, sulfone, sulfonamide, acetyl, optionally substituted C₁-C₆ alkyl, optionally substituted C₁-C₆ alkenyl, optionally substituted C₁-C₆ alkoxy, or hydroxy; and
wherein R₁₅ is sulfonyl, sulfone, sulfonamide, acetyl, optionally substituted C₁-C₆ alkyl, optionally substituted C₁-C₆ alkenyl, optionally substituted C₁-C₆ alkoxy, or hydroxy;
R₃ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide;
R₄ is optionally substituted aryl, optionally substituted heteroaryl, provided that said heteroaryl is not a bicyclic heteroaryl, —C(O)NR₁₆R₁₇, optionally substituted cycloalkyl, optionally substituted cycloalkoxy, optionally substituted C₁-C₆ alkoxy, —NR₁₈R₁₉, optionally substituted C₁-C₆ alkyl, optionally substituted C₂-C₆ alkenyl, optionally substituted C₂-C₆ alkynyl, cyano, —C(O)R₂₀R₂₁, —NR₂₂C(O)R₂₃, —NR₂₂S(O)₂R₂₃, or optionally substituted heterocycloalkyl;
wherein each of R₁₆, R₁₇, R₁₈, R₁₉, R₂₀, R₂₁, R₂₂ and R₂₃ are independently H, optionally substituted C₁-C₆ alkyl, or optionally substituted C₂-C₆ alkenyl;
R₅ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide, provided that R₅ does not include a chemical group comprising a silicon atom; and
R₆ is H, halogen, haloalkyl, C₁-C₆ haloalkoxy, optionally substituted C₁-C₆ alkyl, optionally substituted C₂-C₆ alkenyl, optionally substituted C₂-C₆ alkynyl, optionally substituted aryl, optionally substituted arylalkyl, optionally substituted arylalkenyl, optionally substituted heteroaryl, optionally substituted heteroarylalkyl, optionally substituted heteroarylalkenyl, or —C(O)OH.

In another embodiment, there is provided a compound of Formula 6:

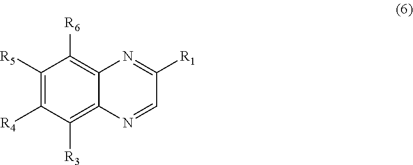

or a pharmaceutically acceptable salt, solvate, tautomer, or prodrug thereof, wherein:
R₁ is optionally substituted C₁-C₆ alkyl, optionally substituted C₁-C₆ alkenyl, optionally substituted aryl, optionally substituted heteroaryl, optionally substituted arylalkyl, optionally substituted heteroarylalkyl, optionally substituted arylalkenyl, —C(O)NZ₁R₇, —NHR₈, —YR₉, —NHYR₉, —C(O)NR₁₀R₁₁, —C═N—NR_{A}R_{B}, and —C═N—O;
wherein Z₁ is H or C₁-C₆ alkyl;
wherein R₇ and R₈ are independently optionally substituted heterocyclylalkyl, optionally substituted heterocyclylalkylalkyl, optionally substituted aryl, optionally substituted arylalkyl, optionally substituted heteroaryl, optionally substituted heteroarylalkyl, optionally substituted heterocyclylalkynyl, optionally substituted heterocyclylalkenyl, optionally substituted arylalkenyl, optionally substituted arylalkynyl, optionally substituted cycloalkyl, optionally substituted cycloalkylalkyl, optionally substituted alkyl, or H;
wherein R₉ is optionally substituted arylalkyl or optionally substituted heteroarylalkyl;
wherein R₁₀ and R₁₁, together with the nitrogen to which they are both attached, form a heterocycle which is optionally substituted;
wherein Y is C(O) or SO₂; and
wherein R_{A} and R_{B} are each independently C₁-C₆ alkyl;
R₃ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide;
R₄ is optionally substituted aryl, optionally substituted heteroaryl, provided that said heteroaryl is not a bicyclic heteroaryl, —C(O)NR₁₆R₁₇, optionally substituted cycloalkyl, optionally substituted cycloalkoxy, optionally substituted C₁-C₆ alkoxy, —NR₁₈R₁₉, optionally substituted C₁-C₆ alkyl, optionally substituted C₂-C₆ alkenyl, optionally substituted C₂-C₆ alkynyl, cyano, —C(O)R₂OR₂₁, —NR₂₂C(O)R₂₃, —NR₂₂S(O)₂R₂₃, optionally substituted heterocycloalkyl;
wherein each of R₁₆, R₁₇, R₁₈, R₁₉, R₂₀, R₂₁, R₂₂ and R₂₃ are independently H, optionally substituted C₁-C₆ alkyl, or optionally substituted C₂-C₆ alkenyl;
R₅ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide, provided that R₅ does not include a chemical group comprising a silicon atom; and
R₆ is H, halogen, haloalkyl, C₁-C₆ haloalkoxy, optionally substituted C₁-C₆ alkyl, optionally substituted C₂-C₆ alkenyl, optionally substituted C₂-C₆ alkynyl, optionally substituted aryl, optionally substituted arylalkyl, optionally substituted arylalkenyl, optionally substituted heteroaryl, optionally substituted heteroarylalkyl, optionally substituted heteroarylalkenyl, or —C(O)OH.

In some embodiments of Formulae (1)-(6), R₃ is H.

In some embodiments of Formulae (1)-(6), R₅ is H or halogen.

In some embodiments of Formulae (1)-(6), R₁ is —(O)NR₁₀R₁₁, and R₁₀ and R₁₁, together with the nitrogen to which they are both attached, form a heterocycle which is optionally substituted.

In another embodiment, there is provided a compound of Formula (7):

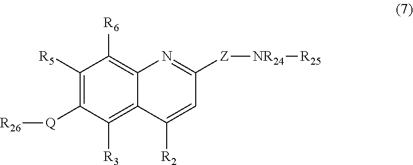

or a pharmaceutically acceptable salt, solvate, tautomer, or prodrug thereof, wherein:
R₂ is H, hydroxyl, cyano, sulfonamide, sulfone, optionally substituted C₁-C₆ alkyl, optionally substituted C₂-C₆ alkene, optionally substituted C₂-C₆ alkyne, optionally substituted C₁-C₆ alkoxy, optionally substituted aryl, optionally substituted heteroaryl, optionally substituted cycloalkyl, optionally substituted heterocycle, NHR₁₂, NR₁₃R₁₄, S(O)₀₋₂R₁₅, or halogen;
R₃ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, and sulfonamide;

### R₅ is H or F;

R₆ is H, halogen, haloalkyl, (C₁-C₆) haloalkoxy, optionally substituted (C₁-C₆) alkyl, optionally substituted (C₂-C₆) alkenyl, optionally substituted (C₂-C₆) alkynyl, optionally substituted aryl, optionally substituted arylalkyl, optionally substituted arylalkenyl, optionally substituted heteroaryl, optionally substituted heteroarylalkyl, optionally substituted heteroarylalkenyl, and —C(O)OH;
Z is carbocyclyl, aryl, O-carbocyclyl, O-aryl, or a 5-6 membered heterocyclyl;

### Q is CH₂, O or S;

R₂₄ is H or optionally substituted (C₁-C₆)alkyl;
R₂₅ is H, (C₁-C₆)alkyl, (C₂-C₆)alkynyl, (C₁-C₆)haloalkyl, (C₂-C₆)haloalkenyl, (C₂-C₆)haloalkynyl, carbocyclyl, aryl, or heterocyclyl and
R₂₆ is H, (C₁-C₆)alkyl, (C₂-C₆)alkenyl, (C₂-C₆)alkynyl, (C₁-C₆)haloalkyl, (C₂-C₆)haloalkenyl, (C₂-C₆)haloalkynyl, carbocyclyl, aryl, or heterocyclyl.

In another embodiment, there is provided a compound of Formula (8):

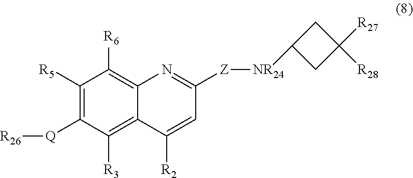

or a pharmaceutically acceptable salt, solvate, tautomer, or prodrug thereof, wherein:
R₂ is H, hydroxyl, cyano, sulfonamide, sulfone, optionally substituted C₁-C₆ alkyl, optionally substituted C₂-C₆ alkene, optionally substituted C₂-C₆ alkyne, optionally substituted C₁-C₆ alkoxy, optionally substituted aryl, optionally substituted heteroaryl, optionally substituted cycloalkyl, optionally substituted heterocycle, NHR₁₂, NR₁₃R₁₄, S(O)₀₋₂R₁₅, or halogen;
R₃ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide;

### R₅ is H or F;

R₆ is H, halogen, haloalkyl, (C₁-C₆) haloalkoxy, optionally substituted (C₁-C₆) alkyl, optionally substituted (C₂-C₆) alkenyl, optionally substituted (C₂-C₆) alkynyl, optionally substituted aryl, optionally substituted arylalkyl, optionally substituted arylalkenyl, optionally substituted heteroaryl, optionally substituted heteroarylalkyl, optionally substituted heteroarylalkenyl, and —C(O)OH;
Z is a 5-6 membered heterocyclyl;

### Q is CH₂, O or S;

R₂₄ is H or optionally substituted (C₁-C₆)alkyl;
R₂₆ is H, (C₁-C₆)alkyl, (C₂-C₆)alkenyl, (C₂-C₆)alkynyl, (C₁-C₆)haloalkyl, (C₂-C₆)haloalkenyl, (C₂-C₆)haloalkynyl, carbocyclyl, aryl, or heterocyclyl.
R₂₇ is H, O, N, S, phosphate, or optionally substituted (C₁-C₆)alkyl; and
R₂₈ is H, (C₁-C₆)alkyl, (C₂-C₆)alkenyl, (C₂-C₆)alkynyl, (C₁-C₆)haloalkyl, (C₂-C₆)haloalkenyl, or (C₂-C₆)haloalkynyl.

In some embodiments of Formula (8),
R₃ is H
Z is a 5-6 membered heterocyclyl;
Q is CH₂, O or S;
R₅ is H;
R₆ is (C₁-C₆)alkyl, (C₂-C₆)alkenyl, (C₂-C₆)alkynyl, (C₁-C₃)haloalkyl, or O—(C₁-C₃)haloalkyl;
R₂₆ is H, (C₁-C₆)alkyl, (C₂-C₆)alkenyl, (C₂-C₆)alkynyl, (C₁-C₆)haloalkyl, (C₂-C₆)haloalkenyl, (C₂-C₆)haloalkynyl, carbocyclyl, aryl, or heterocyclyl.
R₂₇ is H, O, N, S, phosphate, or optionally substituted (C₁-C₆)alkyl; and
R₂₈ is H, (C₁-C₆)alkyl, (C₂-C₆)alkenyl, (C₂-C₆)alkynyl, (C₁-C₆)haloalkyl, (C₂-C₆)haloalkenyl, or (C₂-C₆)haloalkynyl.

In another embodiment, there is provided a compound selected from the group consisting of:

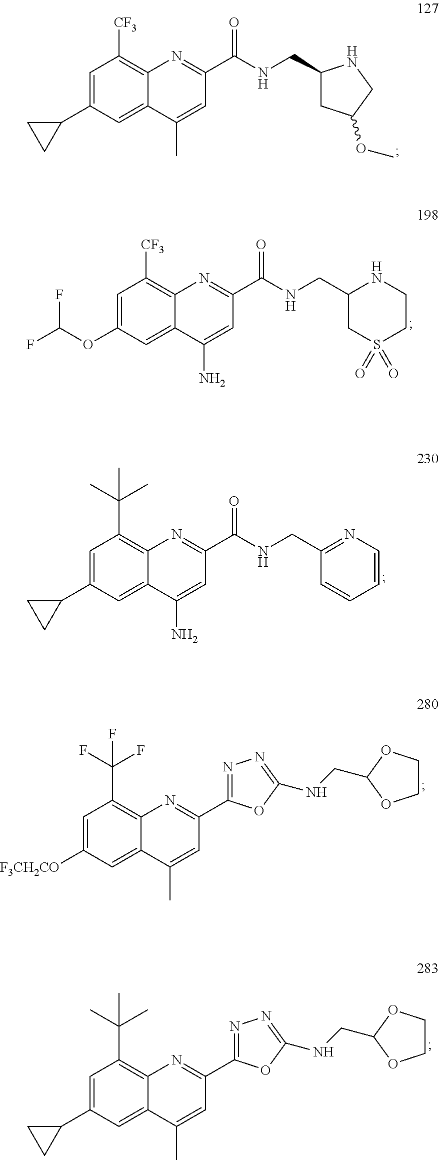

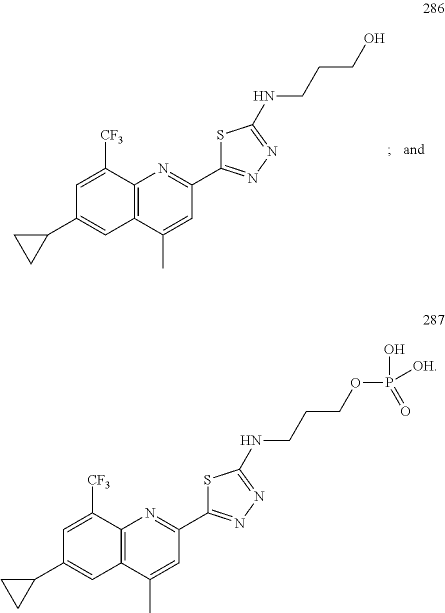

In another embodiment, there is provided a compound selected from the group consisting of:

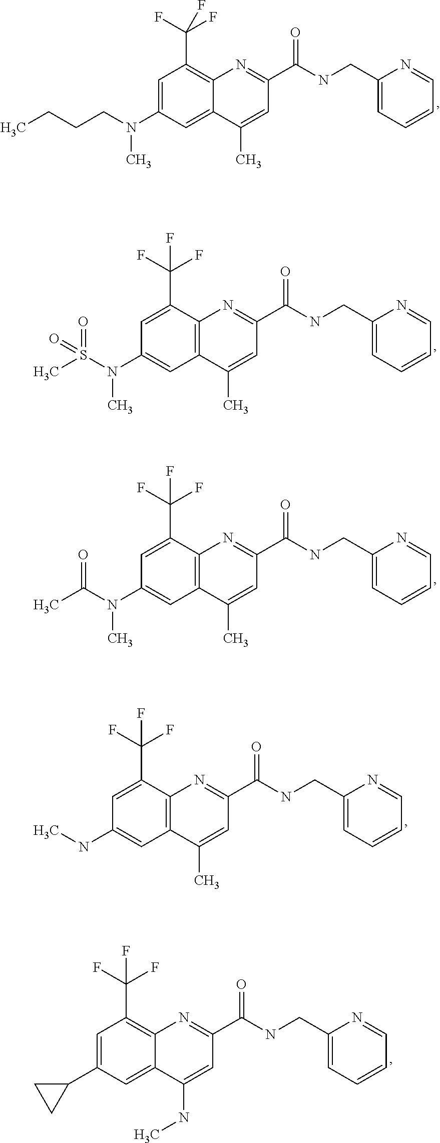

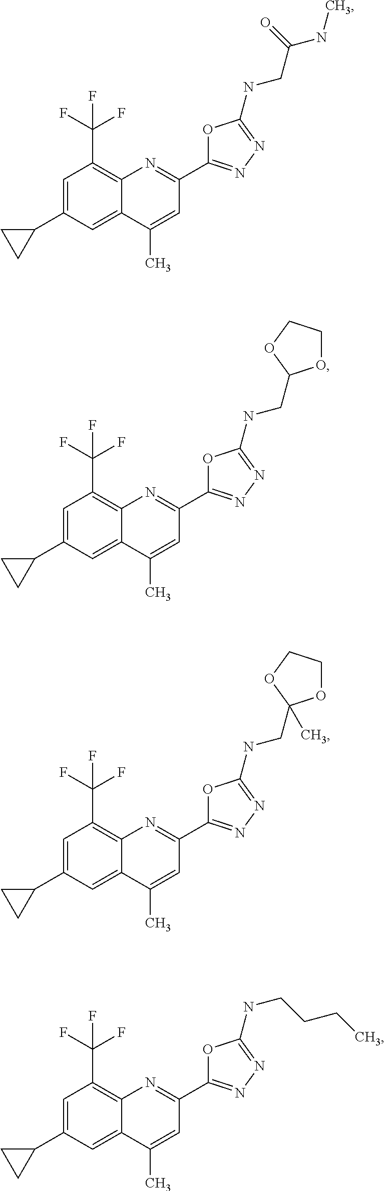

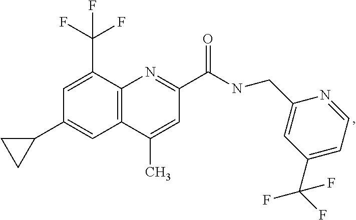

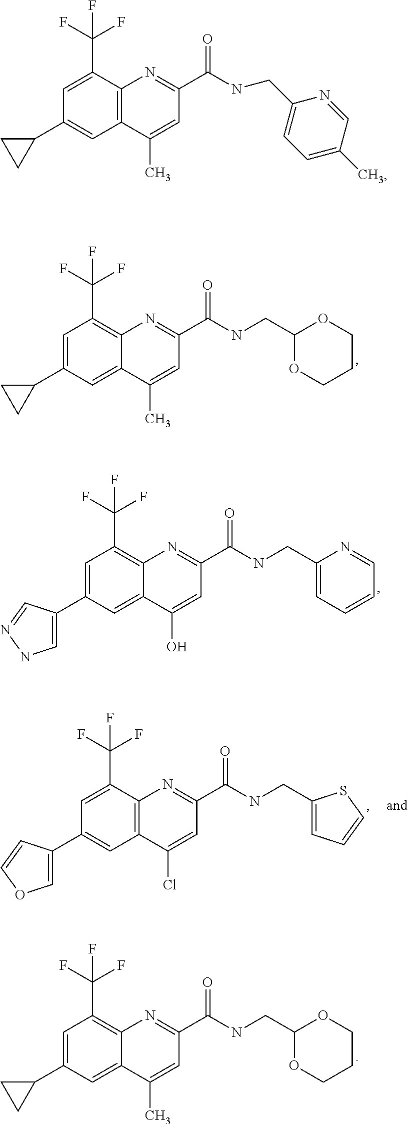

In another embodiment, there is provided a compound selected from the group consisting of:

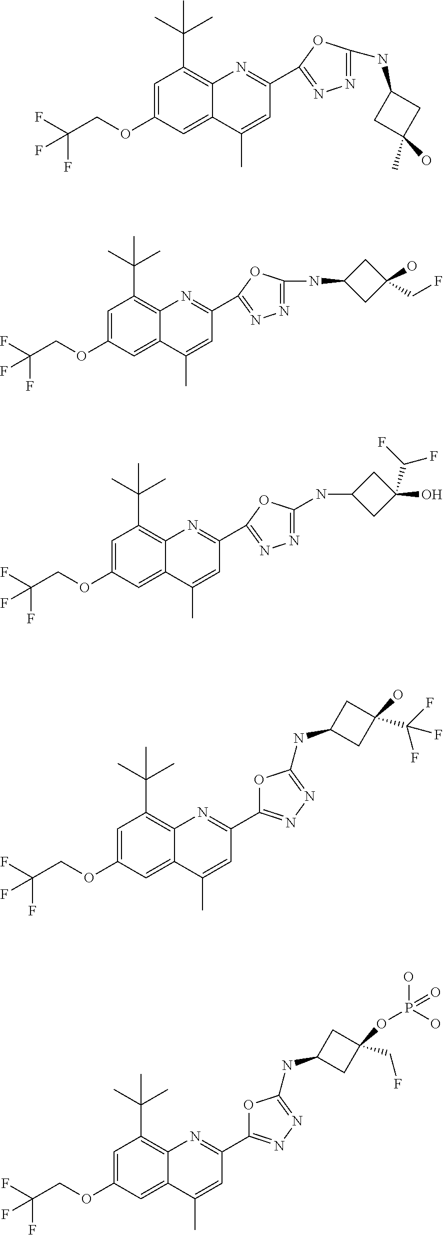

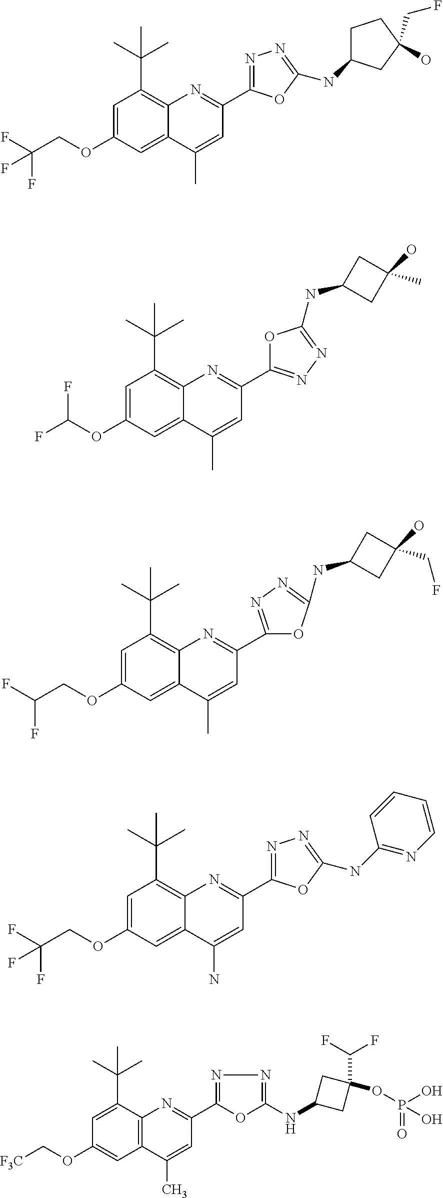

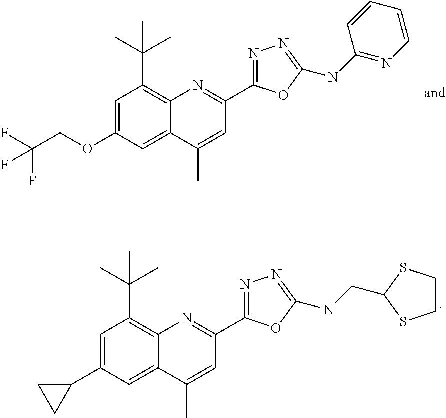

In another embodiment, there is provided a pharmaceutical composition comprising a compound according to any embodiment or example, and one or more pharmaceutically acceptable carrier or excipient.

In another embodiment, there is provided a pharmaceutical composition comprising a compound according to any embodiment or example, and one or more pharmaceutically acceptable carrier or excipient, and further comprising one or more additional therapeutic agent.

In another embodiment, there is provided a method for treating a viral infection comprising administering a compound according to any embodiment or example herein.

In some embodiments, the treatment results in one or more of a reduction in viral load or clearance of RNA.

In another embodiment, there is provided a compound according to any embodiment or example herein for the manufacture of a medicament for the treatment of a viral infection.

In another embodiment, there is provided a compound according to any embodiment or example herein for use in treating a viral infection.

In some embodiments, the treatment results in one or more of a reduction in viral load or clearance of RNA.

In another embodiment, there is provided a method for treating or preventing HCV comprising administering a compound according to any embodiment or example herein.

In another embodiment, there is provided a compound according to any embodiment or example herein for the manufacture of a medicament for the treatment or prevention of HCV.

It will be understood that wherever a hydrogen occurs in a compound of the present invention, the hydrogen can exist as any naturally occurring isotope, such as deuterium.

Another embodiment of the present invention includes a pharmaceutical composition comprising a compound according to the present invention and one or more pharmaceutically acceptable carrier or excipient. In a further embodiment, one or more additional therapeutic agent is also provided in the composition.

Another embodiment of the present invention includes a method for treating a viral infection comprising administering a compound of the present invention. In one embodiment, the treatment results in one or more of a reduction in viral load or clearance of RNA.

Another embodiment of the present invention includes use of a compound of the present invention for the manufacture of a medicament for the treatment of a viral infection. Another embodiment includes a compound for use in treating a viral infection. In one embodiment of each aspect of use and compound, the treatment results in one or more of a reduction in viral load or clearance of RNA.

Another embodiment of the present invention includes a method for treating or preventing HCV comprising administering a compound of the present invention. Another embodiment includes the use of a compound of the present invention for the manufacture of a medicament for the treatment or prevention of HCV.

Another embodiment of the present invention includes pharmaceutical composition comprising a compound of the present invention and one or more pharmaceutically acceptable carrier or excipient. The pharmaceutical composition of the present invention may further comprise one or more additional therapeutic agent. The one or more additional therapeutic agent may be, without limitation, selected from: interferons, ribavirin or its analogs, HCV NS3 protease inhibitors, alpha-glucosidase 1 inhibitors, hepatoprotectants, nucleoside or nucleotide inhibitors of HCV NS5B polymerase, non-nucleoside inhibitors of HCV NS5B polymerase, HCV NS5A inhibitors, TLR-7 agonists, cyclophilin inhibitors, HCV IRES inhibitors, pharmacokinetic enhancers, and other drugs for treating HCV, or mixtures thereof.

Another embodiment of the present invention includes a method for treating a viral infection comprising administering a compound of the present invention. The compound is administered to a human subject in need thereof, such as a human being who is infected with a virus of the Flaviviridae family, such as hepatitis C virus. In one embodiment, the viral infection is acute or chronic HCV infection. In one embodiment, the treatment results in one or more of a reduction in viral load or clearance of RNA.

Another embodiment of the present invention includes the use of a compound according to the present invention for the manufacture of a medicament for the treatment of a viral infection. Another embodiment of the present invention includes a compound according to the present invention for the use in treating a viral infection. In one embodiment, the viral infection is acute or chronic HCV infection. In one embodiment, the treatment results in one or more of a reduction in viral load or clearance of RNA.

The present invention includes combinations of embodiments and embodiments, as well as preferences, as herein described throughout the present specification.

### DETAILED DESCRIPTION

Reference will now be made in detail to certain claims of the invention, examples of which are illustrated in the accompanying structures and formulas. While the invention will be described in conjunction with the enumerated claims, it will be understood that they are not intended to limit the invention to those claims. On the contrary, the invention is intended to cover all alternatives, modifications, and equivalents, which may be included within the scope of the present invention as defined by the claims.

All documents referenced herein are each incorporated by reference in their entirety for all purposes.

### Definitions

Unless stated otherwise, the following terms and phrases as used herein are intended to have the following meanings. The fact that a particular term or phrase is not specifically defined should not be correlated to indefiniteness or lacking clarity, but rather terms herein are used within their ordinary meaning. When trade names are used herein, applicants intend to independently include the tradename product and the active pharmaceutical ingredient(s) of the tradename product.

The term “treating”, and grammatical equivalents thereof, when used in the context of treating a disease, means prophylactic or palliative treatment, or slowing or stopping the progression of a disease, or ameliorating at least one symptom of a disease, more preferably ameliorating more than one symptom of a disease. For example, treatment of a hepatitis C virus infection can include reducing the HCV viral load in an HCV infected human being, and/or reducing the severity of jaundice present in an HCV infected human being.

The term “alcohol,” as used herein, means an aliphatic group wherein one or more hydrogen atoms is replaced by an —OH moiety.

“Alkyl” is hydrocarbon containing normal, secondary, tertiary or cyclic carbon atoms. For example, an alkyl group can have 1 to 20 carbon atoms (i.e., C₁-C₂₀ alkyl), 1 to 10 carbon atoms (i.e., C₁-C₁₀ alkyl), or 1 to 6 carbon atoms (i.e., C₁-C₆ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl 1-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

“Alkoxy” means a group having the formula —O-alkyl, in which an alkyl group, as defined above, is attached to the parent molecule via an oxygen atom. The alkyl portion of an alkoxy group can have 1 to 20 carbon atoms (i.e., C₁-C₂₀ alkoxy), 1 to 12 carbon atoms (i.e., C₁-C₁₂ alkoxy), or 1 to 6 carbon atoms (i.e., C₁-C₆ alkoxy). Examples of suitable alkoxy groups include, but are not limited to, methoxy (—O—CH₃ or —OMe), ethoxy (—OCH₂CH₃ or —OEt), t-butoxy (—O—C(CH₃)₃ or -OtBu), and the like. When an alkyl group is otherwise substituted, for example by a halogen, the alkoxy may be referred to as O-alkyl by way of example, and without limitation, an alkyl trisubstituted with fluorine and attached through an oxygen atom may be referred to as O—CF₃.

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl), 1 to 12 carbon atoms (i.e., C₁-C₁₂ haloalkyl), or 1 to 6 carbon atoms (i.e., C₁-C₆ alkyl). Examples of suitable haloalkyl groups include, but are not limited to, —CF₃, —CHF₂, —CFH₂, —CH₂CF₃, and the like.

“Alkenyl” is a hydrocarbon containing normal, secondary, tertiary, or cyclic carbon atoms with at least one site of unsaturation, i.e. a carbon-carbon, sp2 double bond. For example, an alkenyl group can have 2 to 20 carbon atoms (i.e., C₂-C₂₀ alkenyl), 2 to 12 carbon atoms (i.e., C₂-C₁₂ alkenyl), or 2 to 6 carbon atoms (i.e., C₂-C₆ alkenyl). Examples of suitable alkenyl groups include, but are not limited to, ethylene, vinyl (—CH═CH₂), allyl (—CH₂CH═CH₂), cyclopentenyl (—C₅H₇), and 5-hexenyl (—CH₂CH₂CH₂CH₂CH═CH₂).

“Alkynyl” is a hydrocarbon containing normal, secondary, tertiary or cyclic carbon atoms with at least one site of unsaturation, i.e. a carbon-carbon, sp triple bond. For example, an alkynyl group can have 2 to 20 carbon atoms (i.e., C₂-C₂₀ alkynyl), 2 to 12 carbon atoms (i.e., C₂-C₁₂ alkyne), or 2 to 6 carbon atoms (i.e., C₂-C₆ alkynyl). Examples of suitable alkynyl groups include, but are not limited to, acetylenic (—C═CH), propargyl (—CH₂C—CH), and the like.

“Alkylene” refers to a saturated, branched or straight chain or cyclic hydrocarbon radical having two monovalent radical centers derived by the removal of two hydrogen atoms from the same or two different carbon atoms of a parent alkane. For example, an alkylene group can have 1 to 20 carbon atoms, 1 to 10 carbon atoms, or 1 to 6 carbon atoms. Typical alkylene radicals include, but are not limited to, methylene (—CH₂—), 1,1-ethylene (—CH(CH₃)—), 1,2-ethylene (—CH₂CH₂—), 1,1-propylene (—CH(CH₂CH₃)—), 1,2-propylene (—CH₂CH(CH₃)—), 1,3-propylene (—CH₂CH₂CH₂—), 1,4-butylene (—CH₂CH₂CH₂CH₂—), and the like.

“Alkenylene” refers to an unsaturated, branched or straight chain or cyclic hydrocarbon radical having two monovalent radical centers derived by the removal of two hydrogen atoms from the same or two different carbon atoms of a parent alkene. For example, and alkenylene group can have 1 to 20 carbon atoms, 1 to 10 carbon atoms, or 1 to 6 carbon atoms. Typical alkenylene radicals include, but are not limited to, 1,2-ethylene (—CH═CH—).

“Alkynylene” refers to an unsaturated, branched or straight chain or cyclic hydrocarbon radical having two monovalent radical centers derived by the removal of two hydrogen atoms from the same or two different carbon atoms of a parent alkyne. For example, an alkynylene group can have 1 to 20 carbon atoms, 1 to 10 carbon atoms, or 1 to 6 carbon atoms. Typical alkynylene radicals include, but are not limited to, acetylene (—C≡C—), propargyl (—CH₂C≡C—), and 4-pentynyl (—CH₂CH₂CH₂C≡C—).

When an alkyl, alkenyl or alkynyl group has the generalized prefix (Cₙ-Cₘ), such as, for example, (C₁-C₃)alkyl, it is to be understood that the term provides for the number of carbons in the hydrocarbon chain. Thus, for example, (C₁-C₃)alkyl includes methyl, ethyl, n-propyl and sec-propyl. If the indicated group is optionally substituted, then, for example, the term (C₁-C₃)alkyl would also provide for substituted hydrocarbons of the indicated number of the carbon “backbone,” for illustration, and without limitation, a (C₁-C₃)alkyl optionally substituted with halo would encompass methyl, monofluoromethyl, difluoromethyl, trifluoromethyl, monofluoroethyl, chlorodifluoromethyl, iodoethyl, 2-bromopropyl, and the like.

“Amino” refers to a primary, secondary or tertiary amine group of the generalized formula —NRR′, where when R and R′ are both H, a primary amine is referenced, where either R or R′ is H and the other is not, a secondary amine is referenced, and where both R and R′ are other than H, a tertiary amine is referenced.

“Amido, “carboxamide” and “amide” refer to a group of general formula:

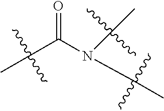

“Aryl” means a monovalent aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 12 carbon atoms. Typical aryl groups include, but are not limited to, radicals derived from benzene (e.g., phenyl), substituted benzene, naphthalene, anthracene, biphenyl, and the like.

“Arylene” refers to an aryl as defined above having two monovalent radical centers derived by the removal of two hydrogen atoms from the same or two different carbon atoms of a parent aryl. Typical arylene radicals include, but are not limited to, phenylene.

“Arylalkyl” refers to an acyclic alkyl radical in which one of the hydrogen atoms bonded to a carbon atom, typically a terminal or sp3 carbon atom, is replaced with an aryl radical. Typical arylalkyl groups include, but are not limited to, benzyl, 2-phenylethan-1-yl, naphthylmethyl, 2-naphthylethan-1-yl, naphthobenzyl, 2-naphthophenylethan-1-yl and the like. The arylalkyl group can comprise 6 to 20 carbon atoms, e.g., the alkyl moiety is 1 to 6 carbon atoms and the aryl moiety is 6 to 14 carbon atoms.

“Aryloxy” refers to an aryl moiety wherein the point or attachment to the adjacent moiety is through an oxygen atom.

The terms “CO” or “carbonyl” as used interchangeably herein, means a ketone of general formula:

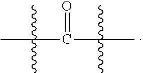

The term “C(O)OH” means a carboxylic acid of general formula:

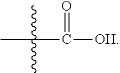

The term C(O)O—R (where R is defined with more particularity by means of a subscript herein) means an ester of general formula:

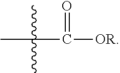

“Cyano” means a carbon atom triple bonded to a nitrogen atom, represented by the formula: —C≡N

“Cycloalkyl” refers to a saturated or partially unsaturated ring having 3 to 7 carbon atoms as a monocycle, 7 to 12 carbon atoms as a bicycle, and up to about 20 carbon atoms as a polycycle. Monocyclic cycloalkyl groups have 3 to 6 ring atoms, still more typically 5 or 6 ring atoms. Bicyclic cycloalkyl groups have 7 to 12 ring atoms, e.g., arranged as a bicyclo (4,5), (5,5), (5,6) or (6,6) system, or 9 or 10 ring atoms arranged as a bicyclo (5,6) or (6,6) system. Cycloalkyl groups include hydrocarbon mono-, bi-, and poly-cyclic rings, whether fused, bridged, or spiro. Non-limiting examples of monocyclic cycloalkyls include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, and the like.

“Cycloalkoxy” refers to a cycloalkyl that is attached to the adjacent moiety through an oxygen atom.

“Cycloalkylene” refers to a cycloalkyl as defined above having two monovalent radical centers derived by the removal of two hydrogen atoms from the same or two different carbon atoms of a parent cycloalkyl. Typical cycloalkylene radicals include, but are not limited to, cyclopropylene and cyclopentylene.

“Halo” or “Halogen” refers to F, Cl, Br, or I.

As used herein the term “haloalkyl” refers to an alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein, the term “haloalkoxy” refers to a group —OR^{a}, where R^{a} is a haloalkyl group as herein defined. As non-limiting examples, haloalkoxy groups include —O(CH₂)F, —O(CH)F₂, O(CHF)Cl, and —OCF₃.

“Heterocycle” or “heterocyclyl” refers to a saturated or partially saturated cyclic group having from 1 to 14 carbon atoms and from 1 to 6 heteroatoms selected from N, S, P, or O, and includes single ring and multiple ring systems including, fused, bridged, and Spiro ring systems. When a “heterocycle” or “heteroaryl” is prefaced by the term “n-membered,” then the total number of atoms, both carbon atoms and heteroatoms, is indicated. Thus, by way of illustrative example, a 5-membered heterocyclyl may include, without limitation, pyrrolidinyl, tetrahydrofuranyl or tetrahydrothiophenyl.

“Heterocycle” or “heterocyclyl” as used herein includes by way of example and not limitation those heterocycles described in Paquette, Leo A.; Principles of Modern Heterocyclic Chemistry (W. A. Benjamin, New York, 1968), particularly Chapters 1, 3, 4, 6, 7, and 9; The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. In one embodiment, the carbon, nitrogen, phosphorous, or sulfur atom(s) of the heterocyclic group may be oxidized to provide for C(═O), N-oxide, phosphinane oxide, sulfinyl, or sulfonyl moieties. As one example, substituted heterocyclyls include, for example, heterocyclic rings substituted with any of the substituents disclosed herein including oxo groups. A non-limiting example of a carbonyl substituted heterocyclyl is:

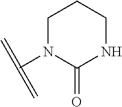

Examples of heterocycles include by way of example and not limitation pyridyl, dihydroypyridyl, tetrahydropyridyl (piperidyl), thiazolyl, tetrahydrothiophenyl, sulfur oxidized tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, piperidinyl, 4-piperidonyl, pyrrolidinyl, azetidinyl, 2-pyrrolidonyl, pyrrolinyl, tetrahydrofuranyl, tetrahydroquinolinyl, tetrahydroisoquinolinyl, decahydroquinolinyl, octahydroisoquinolinyl, azocinyl, triazinyl, 6H-1,2,5-thiadiazinyl, 2H,6H-1,5,2-dithiazinyl, thienyl, thianthrenyl, pyranyl, isobenzofuranyl, chromenyl, xanthenyl, phenoxathinyl, 2H-pyrrolyl, isothiazolyl, isoxazolyl, pyrazinyl, pyridazinyl, indolizinyl, isoindolyl, 3H-indolyl, 1H-indazoly, purinyl, 4H-quinolizinyl, phthalazinyl, naphthyridinyl, quinoxalinyl, quinazolinyl, cinnolinyl, pteridinyl, 4aH-carbazolyl, carbazolyl, β-carbolinyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, piperazinyl, indolinyl, isoindolinyl, quinuclidinyl, morpholinyl, oxazolidinyl, benzotriazolyl, benzisoxazolyl, oxindolyl, benzoxazolinyl, isatinoyl, and bis-tetrahydrofuranyl:

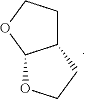

“Heteroaryl” refers to a monovalent aromatic cyclic group having at least one heteroatom in the ring. Thus, “heteroaryl” refers to an aromatic group of from 1 to 14 carbon atoms and 1 to 6 heteroatoms selected from oxygen, nitrogen, sulfur, or phosphorous. For multiple ring systems, by way of example, the term “heteroaryl” includes fused, bridged, and Spiro ring systems having aromatic and non-aromatic rings. In one embodiment, the carbon, nitrogen, or sulfur ring atom(s) of the heteroaryl group may be oxidized to provide for C(═O), N-oxide, sulfinyl, or sulfonyl moieties.

Non-limiting examples of heteroaryl rings include pyridinyl, pyrrolyl, oxazolyl, indolyl, isoindolyl, purinyl, furanyl, thienyl, benzofuranyl, benzothiophenyl, carbazolyl, imidazolyl, thiazolyl, isoxazolyl, pyrazolyl, isothiazolyl, quinolyl, isoquinolyl, pyridazyl, pyrimidyl, pyrazyl, and the like.

“Heterocyclylene” refers to a heterocyclyl, as defined herein, derived by replacing a hydrogen atom from a carbon atom or, as appropriate, a heteroatom of a heterocyclyl, with an open valence. Similarly, “heteroarylene” refers to an aromatic heterocyclylene.

“Heterocyclylalkyl” or “heteroaralkyl” refers to an acyclic alkyl radical in which one of the hydrogen atoms bonded to a carbon atom, typically a terminal or sp3 carbon atom, is replaced with a heterocyclyl radical (i.e., a heterocyclyl-alkylene-moiety). Typical heterocyclyl alkyl groups include, but are not limited to heterocyclyl-CH₂—, 2-(heterocyclyl)ethan-1-yl, and the like, wherein the “heterocyclyl” portion includes any of the heterocyclyl groups described above, including those described in Principles of Modern Heterocyclic Chemistry. One skilled in the art will also understand that the heterocyclyl group can be attached to the alkyl portion of the heterocyclyl alkyl by means of a carbon-carbon bond or a carbon-heteroatom bond, with the proviso that the resulting group is chemically stable. The group comprises 2 to 20 carbon atoms, e.g., the alkyl portion of the group comprises 1 to 6 carbon atoms and the heterocyclyl moiety comprises 3 to 14 members. Examples of heterocyclylalkyls include by way of example and not limitation 5-membered sulfur, oxygen, and/or nitrogen containing heterocycles such as thiazolylmethyl, 2-thiazolylethan-1-yl, imidazolylmethyl, oxazolylmethyl, thiadiazolylmethyl, and the like, 6-membered sulfur, oxygen, and/or nitrogen containing heterocycles such as piperidinylmethyl, piperazinylmethyl, morpholinylmethyl, pyridinylmethyl, pyridizylmethyl, pyrimidylmethyl, pyrazinylmethyl, and the like. Similarly, heteroaralkyl groups include, but are not limited to, —CH₂-pyridinyl, —CH₂-pyrrolyl, —CH₂-oxazolyl, —CH₂-indolyl, —CH₂-isoindolyl, —CH₂-purinyl, —CH₂-furanyl, —CH₂-thienyl, —CH₂-benzofuranyl, —CH₂-benzothiophenyl, —CH₂-carbazolyl, —CH₂-imidazolyl, —CH₂-thiazolyl, —CH₂-isoxazolyl, —CH₂-pyrazolyl, —CH₂-isothiazolyl, —CH₂-quinolyl, —CH₂-isoquinolyl, —CH₂-pyridazyl, —CH₂-pyrimidyl, —CH₂-pyrazyl, —CH(CH₃)-pyridinyl, —CH(CH₃)— pyrrolyl, —CH(CH₃)-oxazolyl, —CH(CH₃)-indolyl, —CH(CH₃)-isoindolyl, —CH(CH₃)— purinyl, —CH(CH₃)-furanyl, —CH(CH₃)-thienyl, —CH(CH₃)-benzofuranyl, —CH(CH₃)— benzothiophenyl, —CH(CH₃)-carbazolyl, —CH(CH₃)-imidazolyl, —CH(CH₃)-thiazolyl, —CH(CH₃)-isoxazolyl, —CH(CH₃)-pyrazolyl, —CH(CH₃)-isothiazolyl, —CH(CH₃)— quinolyl, —CH(CH₃)-isoquinolyl, —CH(CH₃)-pyridazyl, —CH(CH₃)-pyrimidyl, —CH(CH₃)-pyrazyl, and the like.

The term “heterocyclyloxy” represents a heterocyclyl group attached to the adjacent atom by an oxygen.

When there is a sulfur atom present, the sulfur atom can be at different oxidation levels, namely, S, SO, SO₂, or SO₃. All such oxidation levels are within the scope of the present invention.

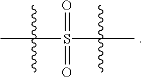

“Sulfonyl” refers to a moiety of general structure

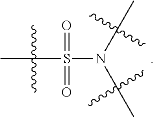

“Aminosulfonyl” refers to a moiety of general structure:

“Alkylsulfonyl” refers to a moiety of general structure:

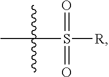

wherein R is an alkyl group as defined herein.

When there is a phosphorous atom present, the phosphorous atom can be at different oxidation levels, namely, POR^{a}R^{b}R^{c}, PO₂R^{a}R^{b}, or PO₃R^{a}R^{b}, where R^{a}, R^{b}, and R^{c} each independently is chosen from H, C₁₋₁₂ alkyl, C₂₋₁₂ alkenyl, C₂₋₁₂ alkynyl, C₆₋₁₄ aryl, 3-12 membered heterocycle, 3-18 membered heteroaralkyl, C₆₋₁₈ aralkyl; or two taken together (with or without oxygens) form a 5 to 10 membered heterocycle. All such oxidation levels are within the scope of the present invention.

A wavy line such as:

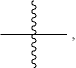

or a double hatched, broken lines such as:

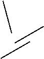

represent a point of attachment of a substituent.

The term “optionally substituted” in reference to a particular moiety of the compound of the Formulae of the invention, for example an “optionally substituted aryl group”, refers to a moiety having none, one, or more substituents.

The term “substituted” in reference to a particular moiety of the compound of the Formulae of the invention, for example, “substituted aryl”, refers to a moiety in which one or more hydrogen atoms are each independently replaced with a non-hydrogen substituent. Divalent groups may also be similarly substituted.

Those skilled in the art will recognize that when moieties such as “alkyl”, “aryl”, “heterocyclyl”, etc. are substituted with one or more substituents, they could alternatively be referred to as “alkylene”, “arylene”, “heterocyclylene”, etc. moieties (i.e., indicating that at least one of the hydrogen atoms of the parent “alkyl”, “aryl”, “heterocyclyl” moieties has been replaced with the indicated substituent(s)). When moieties such as “alkyl”, “aryl”, “heterocyclyl”, etc. are referred to herein as “substituted” or are shown diagrammatically to be substituted (or optionally substituted, e.g., when the number of substituents ranges from zero to a positive integer), then the terms “alkyl”, “aryl”, “heterocyclyl”, etc. are understood to be interchangeable with “alkylene”, “arylene”, “heterocyclylene”, and the like.

As will be appreciated by those skilled in the art, the compounds of the present invention may exist in solvated or hydrated form. The scope of the present invention includes such forms. Again, as will be appreciated by those skilled in the art, the compounds may be capable of esterification. The scope of the present invention includes esters and other physiologically functional derivatives. The scope of the present invention includes prodrug forms of the compound herein described.

“Ester” means any ester of a compound in which any of the —COOH functions of the molecule is replaced by a —C(O)OR function, or in which any of the —OH functions of the molecule are replaced with a —OC(O)R function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof.

The term “prodrug” as used herein refers to any compound that when administered to a biological system generates the drug substance, i.e., active ingredient, as a result of such processes as spontaneous chemical reaction(s), enzyme catalyzed chemical reaction(s), photolysis, and/or metabolic chemical reaction(s). A prodrug is thus a covalently modified analog or latent form of a therapeutically active compound. Example of prodrugs include ester moieties, quaternary ammonium moieties, glycol moieties, and the like.

One skilled in the art will recognize that substituents and other moieties of the compounds of Formula 1 should be selected in order to provide a compound which is sufficiently stable to provide a pharmaceutically useful compound which can be formulated into an acceptably stable pharmaceutical composition. Compounds of Formula 1 which have such stability are contemplated as falling within the scope of the present invention.

As will be appreciated by those skilled in the art, the compounds of the present invention may contain one or more chiral centers. The scope of the present invention includes such forms. Again, as will be appreciated by those skilled in the art, the compound is capable of esterification. The scope of the present invention includes esters and other physiologically functional derivatives. In addition, the scope of the present invention includes prodrug forms of the compounds herein described.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/d iastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

“Enantiomers” refer to stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York.

Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory.

A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates. Thus, where the term “a pharmaceutically acceptable salt, solvate, tautomer, or prodrug thereof” is used, it is to be appreciated that each of these forms is independent of the others, and also includes combinations thereof. For example, the term “a pharmaceutically acceptable salt, solvate, tautomer, or prodrug thereof” includes, without limitation, a pharmaceutically acceptable salt alone, two or more pharmaceutically acceptable salts together, a pharmaceutically acceptable salt and prodrug, a pharmaceutically acceptable salt of a prodrug, and a pharmaceutically acceptable salt which is a solvate, for example. In the case of tautomers, when tautomerization is possible in a compound, a given illustrative chemical structure, even when illustrating only one form, is to be interpreted as including its tautomeric structural form as well.

### Protecting Groups

In the context of the present invention, protecting groups include prodrug moieties and chemical protecting groups.

Protecting groups are available, commonly known and used, and are optionally used to prevent side reactions with the protected group during synthetic procedures, i.e. routes or methods to prepare the compounds of the invention. For the most part the decision as to which groups to protect, when to do so, and the nature of the chemical protecting group “PG” will be dependent upon the chemistry of the reaction to be protected against (e.g., acidic, basic, oxidative, reductive or other conditions) and the intended direction of the synthesis. The PG groups do not need to be, and generally are not, the same if the compound is substituted with multiple PG. In general, PG will be used to protect functional groups such as carboxyl, hydroxyl, thio, or amino groups and to thus prevent side reactions or to otherwise facilitate the synthetic efficiency. The order of deprotection to yield free, deprotected groups is dependent upon the intended direction of the synthesis and the reaction conditions to be encountered, and may occur in any order as determined by the artisan.

Various functional groups of the compounds of the invention may be protected. For example, protecting groups for —OH groups (whether hydroxyl, carboxylic acid, phosphonic acid, or other functions) include “ether- or ester-forming groups”. Ether- or ester-forming groups are capable of functioning as chemical protecting groups in the synthetic schemes set forth herein. However, some hydroxyl and thio protecting groups are neither ether- nor ester-forming groups, as will be understood by those skilled in the art, and are included with amides, discussed below.

A very large number of hydroxyl protecting groups and amide-forming groups and corresponding chemical cleavage reactions are described in Protective Groups in Organic Synthesis, Theodora W. Greene and Peter G. M. Wuts (John Wiley & Sons, Inc., New York, 1999, ISBN 0-471-16019-9) (“Greene”). See also Kocienski, Philip J.; Protecting Groups (Georg Thieme Verlag Stuttgart, New York, 1994), which is incorporated by reference in its entirety herein. In particular Chapter 1, Protecting Groups: An Overview, pages 1-20, Chapter 2, Hydroxyl Protecting Groups, pages 21-94, Chapter 3, Diol Protecting Groups, pages 95-117, Chapter 4, Carboxyl Protecting Groups, pages 118-154, Chapter 5, Carbonyl Protecting Groups, pages 155-184. For protecting groups for carboxylic acid, phosphonic acid, phosphonate, sulfonic acid and other protecting groups for acids see Greene as set forth below. Such groups include by way of example and not limitation, esters, amides, hydrazides, and the like.

### Ether- and Ester-Forming Protecting Groups

Ester-forming groups include: (1) phosphonate ester-forming groups, such as phosphonamidate esters, phosphorothioate esters, phosphonate esters, and phosphon-bis-amidates; (2) carboxyl ester-forming groups, and (3) sulphur ester-forming groups, such as sulphonate, sulfate, and sulfinate.

### Metabolites of the Compounds of the Invention

Also falling within the scope of this invention are the in vivo metabolic products of the compounds described herein. Such products may result for example from the oxidation, reduction, hydrolysis, amidation, esterification and the like of the administered compound, primarily due to enzymatic processes. Accordingly, the invention includes compounds produced by a process comprising contacting a mammal with a compound of this invention for a period of time sufficient to yield a metabolic product of the compound. Such products typically are identified by preparing a radiolabelled (e.g., C¹⁴ or H³) compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS or NMR analysis. In general, analysis of metabolites is done in the same way as conventional drug metabolism studies well-known to those skilled in the art. The conversion products, so long as they are not otherwise found in vivo, are useful in diagnostic assays for therapeutic dosing of the compounds of the invention even if they possess no anti-infective activity of their own.

The definitions and substituents for various genus and subgenus of the present compounds are described and illustrated herein. It should be understood by one skilled in the art that any combination of the definitions and substituents described above should not result in an inoperable species or compound. “Inoperable species or compounds” means compound structures that violates relevant scientific principles (such as, for example, a carbon atom connecting to more than four covalent bonds) or compounds too unstable to permit isolation and formulation into pharmaceutically acceptable dosage forms.

### Pharmaceutical Formulations

The compounds of this invention are typically formulated with conventional carriers and excipients, which will be selected in accord with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986), herein incorporated by reference in its entirety. Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11, but is ordinarily about 7 to 10.

While it is possible for the active ingredients to be administered alone it may be preferable to present them as pharmaceutical formulations. The formulations of the invention, both for veterinary and for human use, comprise at least one active ingredient, together with one or more acceptable carriers and optionally other therapeutic ingredients. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.), herein incorporated by reference in its entirety. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient.

For administration to the eye or other external tissues e.g., mouth and skin, the formulations are preferably applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise one or more compounds of the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth herein, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations suitable for administration to the eye include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10% particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

Formulations suitable for intrapulmonary or nasal administration have a particle size for example in the range of 0.1 to 500 μm (including particle sizes in a range between 0.1 and 500 μm in increments such as 0.5 μm, 1 μm, 30 μm, 35 μm, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents such as compounds heretofore used in the treatment or prophylaxis of infections as described herein.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

Compounds of the invention can also be formulated to provide controlled release of the active ingredient to allow less frequent dosing or to improve the pharmacokinetic or toxicity profile of the active ingredient. Accordingly, the invention also provided compositions comprising one or more compounds of the invention formulated for sustained or controlled release.

The effective dose of an active ingredient depends at least on the nature of the condition being treated, toxicity, whether the compound is being used prophylactically (lower doses) or against an active disease or condition, the method of delivery, and the pharmaceutical formulation, and will be determined by the clinician using conventional dose escalation studies. The effective dose can be expected to be from about 0.001 to about 100 mg/kg body weight per day, typically from about 0.1 to about 50 mg/kg body weight per day, more typically from about 1.0 to about 10 mg/kg body weight per day.

In yet another embodiment, the present application discloses pharmaceutical compositions comprising a compound of Formula 1 or a pharmaceutically acceptable salt thereof, and a pharmaceutically acceptable carrier or excipient.

### Routes of Administration

One or more compounds of the invention (herein referred to as the active ingredients) are administered by any route appropriate to the condition to be treated. Suitable routes include oral, rectal, nasal, topical (including buccal and sublingual), vaginal and parenteral (including subcutaneous, intramuscular, intravenous, intradermal, intrathecal and epidural), and the like. It will be appreciated that the preferred route may vary with for example the condition of the recipient. An advantage of the compounds of this invention is that they are orally bioavailable and can be dosed orally.

### Combination Therapy, Including HCV Combination Therapy

In another embodiment, the compounds of the present invention may be combined with one or more active agent. Non-limiting examples of suitable combinations include combinations of one or more compounds of the present invention with one or more interferons, ribavirin or its analogs, HCV NS3 protease inhibitors, alpha-glucosidase 1 inhibitors, hepatoprotectants, nucleoside or nucleotide inhibitors of HCV NS5B polymerase, non-nucleoside inhibitors of HCV NS5B polymerase, HCV NS5A inhibitors, TLR-7 agonists, cyclophillin inhibitors, HCV IRES inhibitors, pharmacokinetic enhancers, and other drugs for treating HCV.

More specifically, one or more compounds of the present invention may be combined with one or more compounds selected from the group consisting of

1) interferons, e.g., pegylated rIFN-alpha 2b (PEG-Intron), pegylated rIFN-alpha 2a (Pegasys), rIFN-alpha 2b (Intron A), rIFN-alpha 2a (Roferon-A), interferon alpha (MOR-22, OPC-18, Alfaferone, Alfanative, Multiferon, subalin), interferon alfacon-1 (Infergen), interferon alpha-n1 (Wellferon), interferon alpha-n3 (Alferon), interferon-beta (Avonex, DL-8234), interferon-omega (omega DUROS, Biomed 510), albinterferon alpha-2b (Albuferon), IFN alpha XL, BLX-883 (Locteron), DA-3021, glycosylated interferon alpha-2b (AVI-005), PEG-Infergen, PEGylated interferon lambda (PEGylated IL-29), and belerofon,

2) ribavirin and its analogs, e.g., ribavirin (Rebetol, Copegus), and taribavirin (Viramidine),

3) HCV NS3 protease inhibitors, e.g., boceprevir (SCH-503034, SCH-7), telaprevir (VX-950), VX-813, TMC-435 (TMC435350), ABT-450, BI-201335, Bl-1230, MK-7009, SCH-900518, VBY-376, VX-500, GS-9256, GS-9451, BMS-790052, BMS-605339, PHX-1766, AS-101, YH-5258, YH5530, YH5531, and ITMN-191 (R-7227),

4) alpha-glucosidase 1 inhibitors, e.g., celgosivir (MX-3253), Miglitol, and UT-231B,

5) hepatoprotectants, e.g., emericasan (IDN-6556), ME-3738, GS-9450 (LB-84451), silibilin, and MitoQ,

6) nucleoside or nucleotide inhibitors of HCV NS5B polymerase, e.g., R1626, R7128 (R4048), IDX184, IDX-102, PSI-7851, BCX-4678, valopicitabine (NM-283), and MK-0608,

7) non-nucleoside inhibitors of HCV NS5B polymerase, e.g., filibuvir (PF-868554), ABT-333, ABT-072, BI-207127, VCH-759, VCH-916, JTK-652, MK-3281, VBY-708, VCH-222, A848837, ANA-598, GL60667, GL59728, A-63890, A-48773, A-48547, BC-2329, VCH-796 (nesbuvir), GSK625433, BILN-1941, XTL-2125, and GS-9190,

8) HCV NS5A inhibitors, e.g., AZD-2836 (A-831), AZD-7295 (A-689), and BMS-790052,

9) TLR-7 agonists, e.g., imiquimod, 852A, GS-9524, ANA-773, ANA-975, AZD-8848 (DSP-3025), PF-04878691, and SM-360320,

10) cyclophillin inhibitors, e.g., DEBIO-025, SCY-635, and NIM811,

11) HCV IRES inhibitors, e.g., MCI-067,

12) pharmacokinetic enhancers, e.g., BAS-100, SPI-452, PF-4194477, TMC-41629, GS-9350, GS-9585, and roxythromycin,

13) other drugs for treating HCV, e.g., thymosin alpha 1 (Zadaxin), nitazoxanide (Alinea, NTZ), BIVN-401 (virostat), PYN-17 (altirex), KPE02003002, actilon (CPG-10101), GS-9525, KRN-7000, civacir, GI-5005, XTL-6865, BIT225, PTX-111, ITX2865, TT-033i, ANA 971, NOV-205, tarvacin, EHC-18, VGX-410 C, EMZ-702, AVI 4065, BMS-650032, BMS-791325, Bavituximab, MDX-1106 (ONO-4538), Oglufanide, FK-788, and VX-497 (merimepodib).

In yet another embodiment, the present application discloses pharmaceutical compositions comprising a compound of the present invention, or a pharmaceutically acceptable salt thereof, in combination with at least one additional active agent, and a pharmaceutically acceptable carrier or excipient. In yet another embodiment, the present application provides a combination pharmaceutical agent with two or more therapeutic agents in a unitary dosage form. Thus, it is also possible to combine any compound of the invention with one or more other active agents in a unitary dosage form.

The combination therapy may be administered as a simultaneous or sequential regimen. When administered sequentially, the combination may be administered in two or more administrations.

Co-administration of a compound of the invention with one or more other active agents generally refers to simultaneous or sequential administration of a compound of the invention and one or more other active agents, such that therapeutically effective amounts of the compound of the invention and one or more other active agents are both present in the body of the patient.

Co-administration includes administration of unit dosages of the compounds of the invention before or after administration of unit dosages of one or more other active agents, for example, administration of the compounds of the invention within seconds, minutes, or hours of the administration of one or more other active agents. For example, a unit dose of a compound of the invention can be administered first, followed within seconds or minutes by administration of a unit dose of one or more other active agents. Alternatively, a unit dose of one or more other active agents can be administered first, followed by administration of a unit dose of a compound of the invention within seconds or minutes. In some cases, it may be desirable to administer a unit dose of a compound of the invention first, followed, after a period of hours (e.g., 1-12 hours), by administration of a unit dose of one or more other active agents. In other cases, it may be desirable to administer a unit dose of one or more other active agents first, followed, after a period of hours (e.g., 1-12 hours), by administration of a unit dose of a compound of the invention.

The combination therapy may provide “synergy” and “synergistic effect”, i.e. the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e. serially, whereas in combination therapy, effective dosages of two or more active ingredients are administered together.

### Methods of Treatment

Another embodiment of the present invention includes a method for treating a viral infection comprising administering a compound of the present invention. In one embodiment, the treatment results in one or more of a reduction in viral load or clearance of RNA.

Another embodiment of the present invention includes a method for treating or preventing HCV comprising administering a compound of the present invention. Another embodiment includes the use of a compound of the present invention for the manufacture of a medicament for the treatment or prevention of HCV.

Another embodiment of the present invention includes a method for treating a viral infection comprising administering a compound of the present invention. The compound is administered to a human subject in need thereof, such as a human being who is infected with a virus of the Flaviviridae family, such as hepatitis C virus. In one embodiment, the viral infection is acute or chronic HCV infection. In one embodiment, the treatment results in one or more of a reduction in viral load or clearance of RNA.

The effective dose can be expected to be from about 0.001 to about 100 mg/kg body weight per day, typically from about 0.1 to about 50 mg/kg body weight per day, more typically from about 1.0 to about 10 mg/kg body weight per day.

The following Examples illustrate but do not limit the present invention.

### Example 1

### Preparation of Compound 5

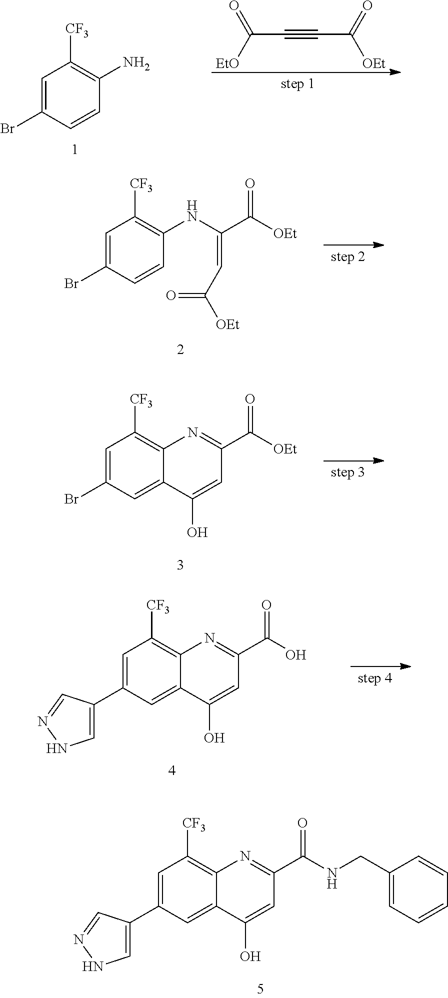

### Step 1

4-Bromo-2-trifluoromethyl-phenylamine (26 g, 0.11 mol)) and But-2-ynedioic acid diethyl ester (20.7 g, 0.12 mol) were dissolved in MeOH (120 ml) in a 500 ml round bottom flask and refluxed. The reaction was monitored by LC-MS. 3 h later; LC-MS showed 4-Bromo-2-trifluoromethyl-phenylamine (15%) was still present, then 0.1 eq. But-2-ynedioic acid diethyl ester was added after reaction cooling down. 2 h later, LC-MS did not shown much progress. The reaction mixture was concentrated down to remove the solvent under vacuum, thick oil was obtained and it was used as crude for the next step. MS [M+H]⁺=411.8 (100%), 409.8 (90%)

### Step 2

A sand bath was heated to 400° C. The crude material from previous step was charged in Ph₂O (100 ml) in a 500 ml round bottom flask with mouth open, the reaction mixture was placed in the preheated sand bath and inner temperature was monitored. After 1 h, the inner temperature reached to 240° C., and the solution color changed from yellow to green then to brown during inner temperature rising. When inner temperature reached 240° C., every 3 minutes the reaction was monitored by LC-MS, when no SM was left, remove the heat. White solid which is product crashed out when solution cooled to room temperature. Solid was filtered and washed with hexane, mother liquid was concentrated and more solid crashed out, repeat above procedure to recover more product. After 3 times repeating, product was obtained in 17 g. MS [M+H]⁺=366.0 (100%), 364.0 (98%).

### Step 3

To a mixture of compound 3 (0.4 g, 1.1 mmol), 4-(3,3,4,4-Tetramethyl-borolan-1-yl)-pyrazole-1-carboxylic acid tert-butyl ester (0.64 g, 2.2 mmol), Pd(PPh₃)₄ (0.13 g, 0.11 mmol) in a microwave tube was added dioxane (5 ml) and K₃PO₄ (1M) (3.3 ml). The reaction mixture was placed in microwave reactor at 120° C. for 30 minutes. Pd catalyst was filtered off. When the mixture was acidified with HCl (2N) to PH=4, solid product 4 was precipitated out. The filter cake was washed with water followed by hexane, and dried under high vacuum to afford light brown color solid. The crude material was taken forward to next step without further purification. 400 MHz ¹H NMR (DMSO): 8.49 (s, 1H), 8.36 (s, 1H), 8.31 (s, 2H), 7.42 (s, 1H). MS[M+H]=324; LCMS RT=1.63 min

### Step 4

Acid 4 (0.02 g, 0.06 mmol) and benzylamine (0.01 g, 0.12 mmol) were dissolved in DMF (1.5 ml), followed by the addition of EDCl (0.03 g, 0.16 mmol), HOBt (0.02 g, 0.16 mmol), and NMM (0.02 g, 0.25 mmol). The reaction was stirred at rt for overnight, and monitored by LC-MS. Reaction mixture was purified by prep-HPLC to afford light brown solid 5 (0.01 g, 0.02 mmol). ¹H-NMR (400 MHz, DMSO-d6) δ 12.26 (bs, 1H), 8.66 (m, 1H), 8.53 (m, 1H), 8.40 (s, 1H), 8.34 (m, 1H), 7.61 (s, 1H), 7.32 (m, 4H), 7.23 (m, 1H), 4.58 (d, 2H). ¹⁹F NMR (376.1 MHz) δ −58.56 (s), 73.98 (s), MS [M+H]⁺=413.1

### Example 2

### Preparation of Compound 7

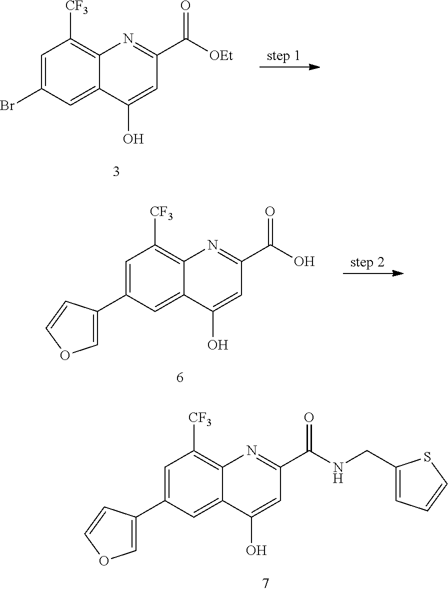

### Step 1

The procedure was same as step 3 in Example 1 to afford compound 6. MS [M+H]⁺=324.0

### Step 2

Acid 6 (534 mg, 1.65 mmol) dissolved in DMF (8 ml) was added NMM (545 ul, 4.96 mmol) and HATU (942 mg, 2.48 mmol) at RT under N₂. After stirred for 5 min, C-thiophen-2-yl-methylamine (374 mg, 3.30 mmol) was added. The reaction was stirred at RT for overnight until completion. Reaction mixture was diluted with EtOAc, washed with 3% LiCl (aq), sat'd NaHCO₃ and brine. The organic layer was dried (Na₂SO₄) and concentrated. The crude product was purified by flash chromatography on silica gel with EA/Hex to give 386 mg (56%) of compound 7. ¹H-NMR (400 MHz, DMSO-d6) δ 12.39 (bs, 1H), 8.68 (t, 1H), 8.53 (s, 2H), 8.40 (s, 1H), 7.80 (t, 1H), 7.62 (s, 1H), 7.39 (m, 1H), 7.05 (m, 1H), 6.96 (m, 1H), 4.74 (d, 2H); ¹⁹F NMR (376.1 MHz) δ −58.59 (s); MS [M+H]⁺=418.9.

### Example 3

### Preparation of Compounds 8-20

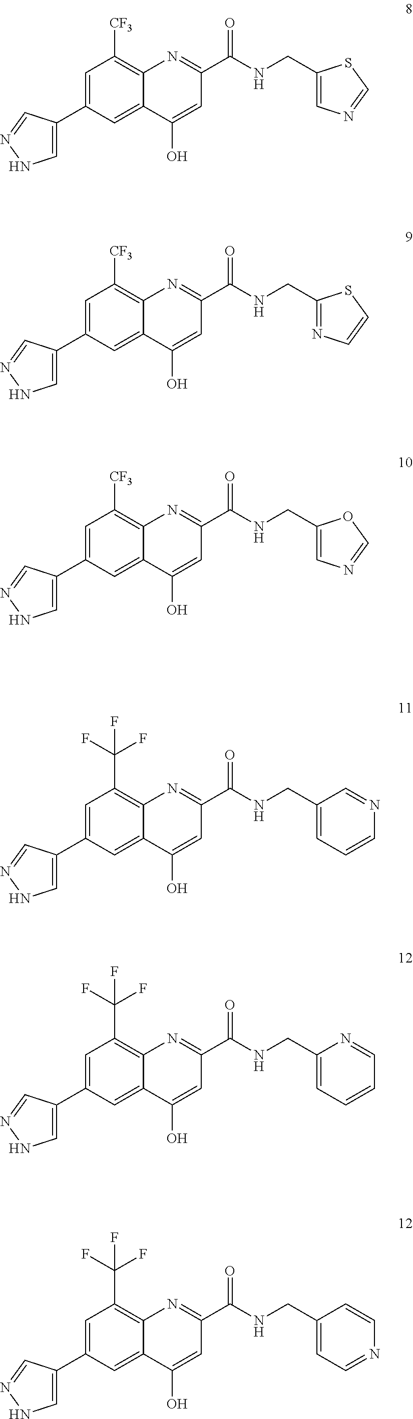

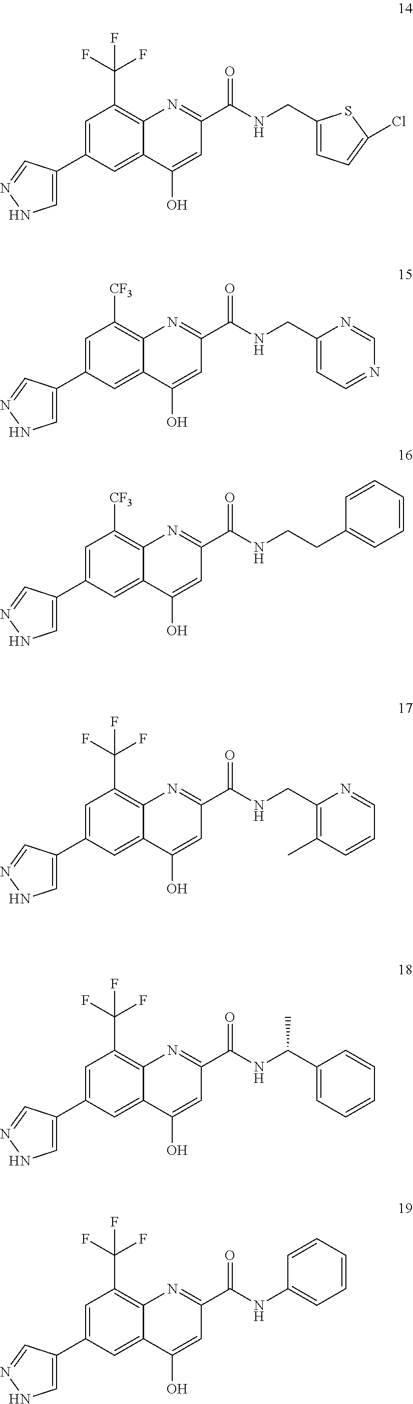

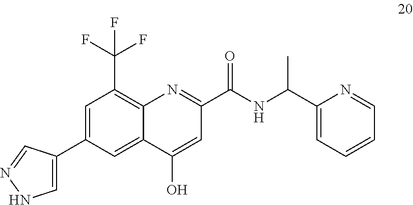

The compounds in the example were made according to procedures in example 1.

Compound 8: ¹H-NMR (400 MHz, DMSO-d6) δ 12.24 (bs, 1H), 8.93 (m, 1H), 8.73 (m, 1H), 8.49 (s, 1H), 8.37 (s, 1H), 8.31 (m, 2H), 7.80 (s, 1H), 7.56 (m, 1H), 4.75 (d, 2H). ¹⁹F NMR (376.1 MHz) δ −58.47 (s), −74.59 (s); MS [M+H]⁺=420.1.

Compound 9: ¹H-NMR (400 MHz, DMSO-d6) δ 12.30 (bs, 1H), 8.99 (m, 1H), 8.54 (m, 1H), 8.42 (s, 1H), 8.36 (m, 2H), 8.42 (m, 1H), 8.36, (m, 2H), 7.74 (m, 1H), 7.61 (m, 2H), 4.88 (d, 2H). ¹⁹F NMR (376.1 MHz) δ −58.46 (s), −74.55 (s); MS [M+H]⁺=420.1.

Compound 10: ¹H-NMR (400 MHz, DMSO-d6) δ 12.27 (bs, 1H), 8.53 (m, 2H), 8.41 (s, 1H), 8.35 (m, 2H), 8.00 (s, 1H), 7.60 (s, 1H), 4.49 (d, 2H). ¹⁹F NMR (376.1 MHz) δ −58.58 (s), −74.09 (s); MS [M+H]⁺=404.1.

Compound 11: ¹H-NMR (400 MHz, DMSO-d6) δ 12.26 (bs, 1H), 8.83 (m, 1H), 8.67 (s, 1H), 8.57 (m, 1H), 8.54 (m, 2H), 8.48 (m, 1H), 8.40 (s, 1H), 8.35 (m, 2H), 8.01 (m, 1H), 7.59 (m, 2H), 4.65 (d, 2H). ¹⁹F NMR (376.1 MHz) δ −58.43 (s), −74.17 (s); MS [M+H]⁺=414.1.

Compound 12: ¹H-NMR (400 MHz, DMSO-d6) δ 12.28 (bs, 1H), 9.08 (m, 1H), 8.61 (m, 1H), 8.54 (m, 1H), 8.42 (m, 1H), 8.36 (m, 2H), 7.96 (m, 1H), 7.60 (s, 1H), 7.51 (m, 1H), 7.44 (m, 1H), 4.65 (d, 2H). ¹⁹F NMR (376.1 MHz) δ −58.51 (s), −74.66 (s); MS [M+H]⁺=414.1.

Compound 13: ¹H-NMR (400 MHz, DMSO-d6) δ 12.31 (bs, 1H), 8.92 (m, 1H), 8.66 (m, 2H), 8.55 (s, 1H), 8.42 (s, 1H), 8.36 (s, 1H), 7.68 (m, 2H), 7.60 (s, 1H), 4.74 (d, 2H). ¹⁹F NMR (376.1 MHz) δ −58.38 (s), −74.08 (s); MS [M+H]⁺=414.1.

Compound 14: ¹H-NMR (400 MHz, DMSO-d6) δ 13.12 (bs, 1H), 8.75 (m, 2H), 8.58 (s, 1H), 7.86 (s, 1H), 7.53 (m, 1H), 6.98-6.91 (m, 3H), 4.64 (d, 2H). ¹⁹F NMR (376.1 MHz) δ −58.73 (s); MS [M+H]⁺=453.0.

Compound 15: ¹H-NMR (400 MHz, DMSO-d6) δ 12.27 (bs, 1H), 9.11 (m, 1H), 9.02 (s, 1H), 8.71 (m, 1H), 7.55 (m, 1H), 8.43 (s, 1H), 8.36 (m, 2H), 7.60 (s, 1H), 7.44 (m, 1H), 4.69 (d, 2H). ¹⁹F NMR (376.1 MHz) δ −58.48 (s), −74.55 (s); MS [M+H]⁺=415.1.

Compound 16: ¹H-NMR (400 MHz, DMSO-d6) δ 12.20 (bs, 1H), 8.49 (m, 1H), 8.36 (s, 1H), 8.31 (s, 1H), 8.18 (b, 1H), 7.54 (s, 1H), 7.20 (m, 4H), 7.15 (m, 1H), 3.57 (m, 2H), 2.82 (d, 2H). ¹⁹F NMR (376.1 MHz) δ −58.65 (s), −74.55 (s); MS [M+H]⁺=427.2.

Compound 17: ¹H-NMR (400 MHz, DMSO-d6) δ 12.28 (br, 1H), 9.441 (br, 1H), 8.54 (d, 1H), 8.46 (d, 1H), 8.437 (s, 1H), 8.36 (s, 2H), 7.85 (m, 1H), 7.61 (s, 1H), 7.43 (m, 1H), 4.72 (d, 2H), 2.38 (s, 3H). ¹⁹F NMR (376.1 MHz) δ −58.61 (s), −74.58 (s); MS [M+H]⁺=428.1.

Compound 18: ¹H-NMR (400 MHz, DMSO-d6) δ 12.30 (br, 1H), 8.53 (m, 2H), 8.42 (s, 1H), 8.32 (m, 2H), 7.55 (s, 1H), 7.37 (m, 4H), 7.26 (m, 1H), 5.12 (m, 1H), 1.50 (d, 3H). ¹⁹F NMR (376.1 MHz) δ −58.79 (s), −74.52 (s); MS [M+H]⁺=427.0.

Compound 19: ¹H-NMR (400 MHz, DMSO-d6) δ 12.47 (br, 1H), 10.19 (s, 1H), 8.57 (d, 1H), 8.47 (s, 1H), 8.38 (m, 2H), 7.70 (d, 2H), 7.66 (s, 1H), 7.42 (m, 2H), 7.17 (m, 1H). ¹⁹F NMR (376.1 MHz) δ −58.68 (s), −74.09 (s); MS [M+H]⁺=400.

Compound 20: ¹H-NMR (400 MHz, DMSO-d6) δ 12.30 (br, 1H), 9.19 (d, 1H), 8.60 (d, 1H), 8.53 (d, 1H), 8.43 (s, 1H), 8.36 (s, 2H), 7.90 (t, 1H), 7.56 (m, 2H), 7.40 (m, 1H), 5.22 (m, 1H), 1.51 (d, 3H). ¹⁹F NMR (376.1 MHz) δ −58.72 (s), −74.09 (s); MS [M+H]⁺=400.

### Example 4

### Preparation of Compound 23

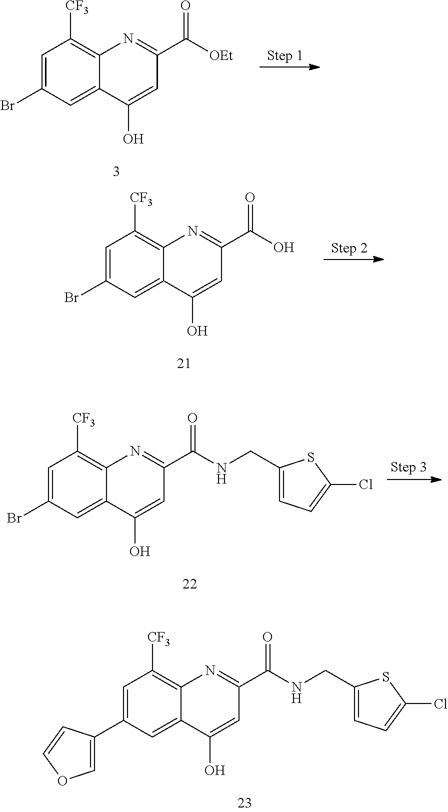

### Step 1

6-Bromo-4-hydroxy-8-trifluoromethyl-quinoline-2-carboxylic acid ethyl ester 3 from example 1 (360 mg, 0.99 mmol) was dissolved in THF/MeOH (5 ml/2 ml), followed by the addition of LiOH aqueous solution (1 N) (5 ml). reaction mixture was stirred at rt for 2 h, and it was monitored by LC-MS. Desired product was crashed out when reaction mixture was acidified by 2N HCl to PH=3. Solid was filtered off and washed with H₂O and followed by hexane, the solid was dried under high vacuum and compound 21 (0.2 g, 0.6 mmol, 60%) was obtained. LC-MS (M+1)=336.0

### Step 2

Acid 21 (0.16 g, 0.48 mmol) and C-(5-Chloro-thiophen-2-yl)-methylamine HCl salt (0.18 g, 0.96 mmol) were dissolved in DMF (2 ml), followed by the addition of EDCl (0.23 g, 1.2 mmol), HOBt (0.16 g, 1.2 mmol), and NMM (0.19 g, 1.9 mmol). The reaction was stirred at rt for overnight, and monitored by LC-MS. LiCl (5%) was added to the reaction mixture, product was precipitated out. Solid was filtered off and washed with H₂O and Hexane, dried under high vacuum to obtain desired product (0.14 g, 0.30 mmol) in brown color.

LC-MS (M+1)=466.7

### Step 3

To a mixture of compound 22 (0.07 g, 0.15 mmol), 3-furan boronic acid (0.017 g, 0.15 mmol), Pd(PPh₃)₄ (0.008 g, 5% loading) in a microwave tube was added Dioxane (1 ml) and followed by K₃PO₄ (1M) (1 ml). The reaction mixture was subjected in microwave at 120° C. for 10 minutes. Crude material was purified by pre-HPLC to afford white solid 23 (0.01 g, 0.02 mmol). ¹H-NMR (400 MHz, DMSO-d6) δ 12.37 (bs, 1H), 8.76 (m, 2H), 8.53 (m, 2H), 8.40 (s, 1H), 7.81 (s, 1H), 7.61 (m, 2H), 7.20 (s, 1H), 6.94 (m, 1H), 6.91 (m, 1H), 4.65 (d, 2H). ¹⁹F NMR (376.1 MHz) δ −58.52 (s), 73.45 (s), 117.0 (m) (TFA); MS [M+H]⁺=451.2.

### Example 5

### Preparation of Compounds 25 and 26

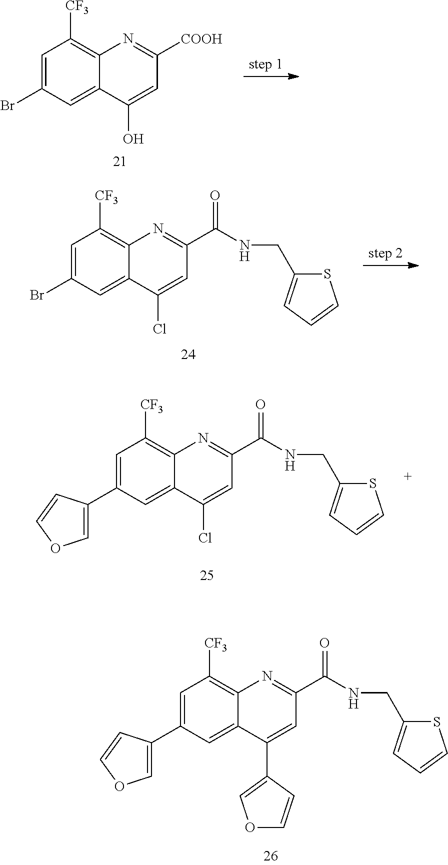

### Step 1

A 100-mL 1-neck rbf was charged with intermediate 21 from example 4 (0.92 g, 2.75 mmol), 15 mL thionyl chloride and DMF (4 drops). The reaction mixture was heated up to reflux for 2 hs with stirring. Excess thionyl chloride was then removed in vacuo, and the residue co-evaporated with toluene (2×20 mL). The residue was dissolved in DMF (10 mL) and was added 2-thiophene methylamine (0.37 g, 3.3 mmol) and NMM (0.36 g, 3.58 mmol). The reaction mixture was stirred at room temperature for 1 h and diluted with EtOAc (100 mL) and washed with 5% LiCl and dried with sodium sulfate. After removal of the solvent in vacuo, the residue was purified by preparative flash chromatography (silica gel, ethyl acetate/hexane gradient) affording 0.6 g of intermediate 24 as a white solid. ¹H-NMR (400 MHz, CH₃OH-d₄) δ 8.76 (s, 1H), 8.42 (s, 1H), 8.37 (s, 1H), 7.33 (m, 1H), 7.09 (m, 1H), 6.96 (m, 1H), 4.84 (s, 2H); ¹⁹F NMR (376.1 MHz, CH₃OH-d₄) δ −60.21 (s); MS [M+H]⁺=449.9.

### Step 2

A 25-mL microwave tube was charged with intermediate 24 (0.6 g, 1.34 mmol), 3-furanboronic acid (0.3 g, 2.68 mmol), tetrakis(triphenylphosphine)palladium(0) (0.078 g, 0.068 mmol), 1M potassium phosphate (3 mL) and dioxane (5 mL). The reaction mixture was heated up to 140° C. under microwave with stirring for 10 mins. The reaction mixture was diluted with EtOAc (100 mL) and washed with water (2×50 mL) and dried with sodium sulfate. After removal of the solvent in vacuo, the residue was purified by preparative flash chromatography (silica gel, ethyl acetate/hexane gradient) affording 0.29 g of compound 25 and 0.08 g of compound 26, both as white solids. Compound 25: ¹H-NMR (400 MHz, CCl₃H-d) δ 8.44 (m, 2H), 8.23 (s, 1H), 7.95 (s, 1H), 7.56 (m, 1H), 7.29 (m, 1H), 7.06 (m, 1H), 6.97 (m, 1H), 6.85 (m. 1H), 4.88 (m, 2H); ¹⁹F NMR (376.1 MHz, CCl₃H-d) δ −60.07 (s); MS [M+H]⁺=436.8.

Compound 26: ¹H-NMR (400 MHz, CCl₃H-d) δ 8.59 (m, 1H), 8.41 (m, 1H), 8.36 (s, 1H), 8.22 (s, 1H), 7.88 (s, 1H), 7.83 (s, 1H), 7.66 (m, 1H), 7.53 (m, 1H), 7.23 (m, 1H), 7.08 (m, 1H), 6.97 (m, 1H), 6.79 (m. 2H), 4.1 (d, J=6.4 Hz, 2H); ¹⁹F NMR (376.1 MHz, CCl₃H-d) δ −60.11 (s); MS [M+H]⁺=468.9

### Example 6

### Preparation of Compound 27

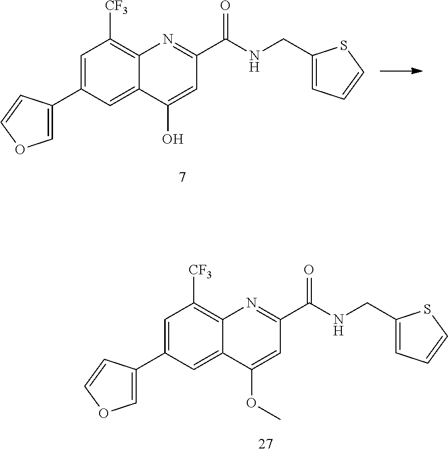

Compound 7 from example 2 (102 mg, 0.244 mmol) dissolved in DMF (2 ml) was added K₂CO₃ (41.3 mg, 0.293 mmol) followed by MeI (38 mg, 0.268 mmol) dropwise at RT under N₂. After stirred for 2 h, more of K₂CO₃ (41.3 mg, 0.293 mmol) and MeI (38 mg, 0.268 mmol) were added. The reaction was stirred at RT for another 1 h for completion. Reaction mixture was diluted with EtOAc, washed with 3% LiCl (aq), sat'd NaHCO₃ and brine. The organic layer was dried (Na₂SO₄) and concentrated. The crude product was purified by flash chromatography on silica gel with EA/Hex to give 76 mg (72%) of compound 27. ¹H-NMR (400 MHz, CHCl₃-d) δ 8.63 (m, 1H), 8.44 (m, 1H), 8.17 (s, 1H), 7.91 (s, 1H), 7.80 (s, 1H), 7.54 (m, 1H), 7.23 (m, 1H), 7.07 (m, 1H), 6.97 (m, 1H), 6.84 (m, 1H), 4.88 (d, 2H), 4.16 (s, 3H); ¹⁹F NMR (376.1 MHz) δ −60.18 (s); MS [M+H]⁺=433.0. 77

### Example 7

### Preparation of Compound 28

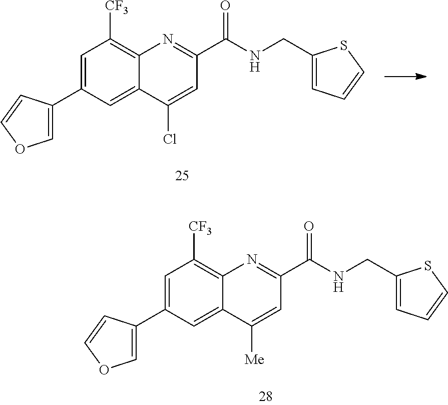

Intermediate 25 from Example 5 (16 mg, 0.037 mmol) was suspended in dioxane (0.5 ml) in a small microwave vial with [1,1′bis(diphenylphosphino)ferrocene]palladium(II) chloride (1:1 complex with DCM) (1.5 mg, 0.002 mmol). The vial was sealed, placed under N₂ and treated with a solution of Me₂Zn in THF (90 uL, 0.18 mmol, 2.0 M). The homogeneous solution was heated at 100° C. for 5 h. The reaction was then cooled to rt, diluted with DMF and purified by RP-HPLC. Lyophilization provided the desired product 28 as a white powder (6.9 mg, 34% yield). ¹H-NMR (400 MHz, CHCl₃-d) δ 8.56 (t, J=6 Hz, 1H), 8.439 (s, 1H), 8.30 (d, J=5 Hz, 1H), 8.10 (s, 1H), 7.99 (s, 1H), 7.63 (s, 1H), 7.23 (d, J=6 Hz, 1H), 7.01 (s, 1H), 7.00 (m, 1H), 6.89 (m 1H), 4.77 (d, J=6 Hz, 2H), 2.82 (s, 3H); MS [M+H]⁺=416.91

### Example 8

### Preparation of Compounds 29-31

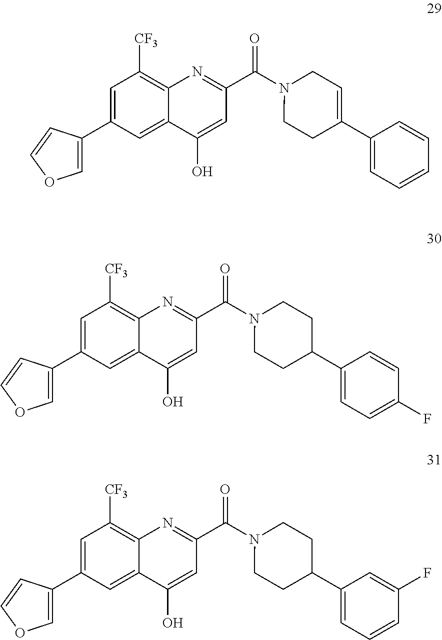

The compounds in the example were made from compound 6 according to the procedure in step 2 of example 2.

Compound 29: ¹H-NMR (400 MHz, DMSO-d6) δ 12.23 (sb, 1H), 8.53 (m, 2H), 8.39 (s, 1H), 7.81 (s, 1H), 7.42 (m, 2H), 7.37-7.16 (m, 5H), 4.31 (m, 2H), 3.89 (m, 1H), 3.78 (m, 2H), 2.61 (m, 2H). ¹⁹F NMR (376.1 MHz) δ −58.64 (s), −58.69 (s); MS [M+H]⁺=465.0.

Compound 30: ¹H-NMR (400 MHz, DMSO-d6) δ 12.20 (bs, 1H), 8.55 (m, 2H), 8.40 (m, 1H), 7.83 (m, 1H), 7.31 (m, 2H), 7.22 (m, 1H), 7.14 (m, 3H), 4.69 (d, 1H), 4.20 (d, 1H), 3.15 (m, 1H), 2.90 (m, 2H), 1.91 (m, 1H), 1.74 (m, 2H), 1.65 (m, 1H); ¹⁹F NMR (376.1 MHz) δ −58.76 (s), −73.94 (s), −117.0 (m) (TFA salt); MS [M+H]⁺=485.20.

Compound 31: ¹H-NMR (400 MHz, DMSO-d6) δ 12.20 (bs, 1H), 8.55 (m, 2H), 8.40 (m, 1H), 7.83 (m, 1H), 7.37 (m, 2H), 7.22 (m, 1H), 7.17-7.09 (m, 3H), 7.06-7.01 (m, 1H), 4.69 (d, 2H), 4.20 (d, 1H), 3.15 (m, 1H), 2.90 (m, 2H), 1.93 (m, 1H), 1.76 (m, 2H), 1.67 (m, 1H); ¹⁹F NMR (376.1 MHz) δ −58.76 (s), −73.95 (s), −113.30 (m) (TFA salt); MS [M+H]⁺=485.20.

### Example 9

### Preparation of Compounds 34-36

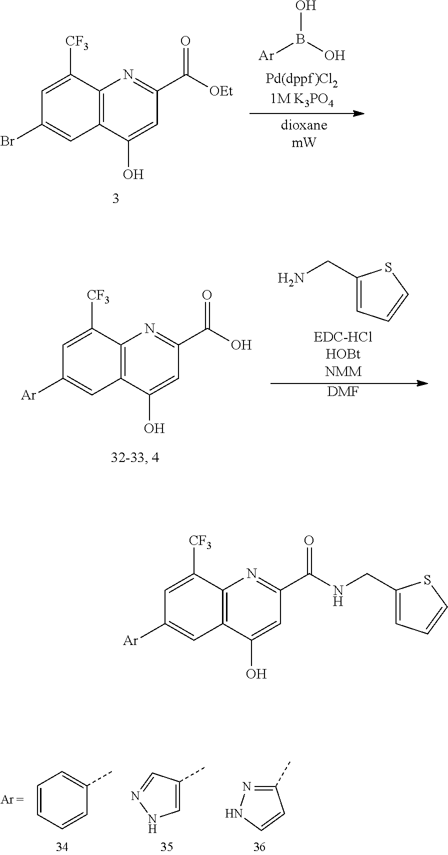

### Step 1

A 20-mL microwave vial equipped with a stir bar was loaded with Intermediate 3 from example 1 (300 mg, 0.824 mmol), phenyl boronic acid (138 mg, 1.24 mmol) and Pd(dppf)Cl₂ (67 mg, 0.082 mmol). Aqueous 1M K₃PO₄ (2.5 mL, 2.5 mmol) and dioxane (8 mL) were added to the mixture. The vial was sealed and subjected to heating in a microwave oven at 100° C. for 15 min. Analysis by LC/MS revealed the presence of desired product and some starting material. The vial was re-sealed and subjected to heating at the same temperature for an additional hour. Complete conversion to the desired product 32 was observed. The reaction mixture was concentrated under vacuum, and the residue was treated with 5 mL of aqueous 1M HCl. The resulting solid (210 mg, 73% yield) was used in the next step. An analytical sample was purified by HPLC. Compound 33 was synthesized in a similar manner. The preparation of compound 4 was described in example 1.

Compound 32: 400 MHz ¹H NMR (DMSO): 8.58 (s, 1H), 8.38 (s, 1H), 7.82-7.81 (d, 2H), 7.51-7.47 (m, 3H), 7.43-7.39 (t, 1H). MS[M+H]=334; LCMS RT=2.06 min

Compound 33: MS[M+H]=324; LCMS RT=1.63 min

### Step 2

A 8-mL vial equipped with a stir bar was loaded with compound 32 (110 mg, 0.329 mmol), EDC hydrochloride (148 mg, 0.772 mmol), HOBt (104 mg, 0.770 mmol) and N-methyl morpholine (70 uL, 0.670 mmol). To this mixture anhydrous DMF (3 mL) was added, followed by the addition of 2-aminomethyl thiophene (140 uL, 1.24 mmol). The reaction mixture was stirred for 1 hour at room temperature. Analysis by LC/MS showed complete conversion of the starting material to the desired product. The reaction mixture was purified using preparative HPLC to give the final compound 34 as the trifluoroacetate salt (53 mg, 38% yield). Compounds 35 and 36 were synthesized in a similar manner from compounds 4 and 33, respectively.

Compound 34: 400 MHz ¹H NMR (DMSO): 12.46 (s, 1H), 8.72 (s, 1H), 8.61 (s, 1H), 8.40 (s, 1H), 7.85 (d, 2H), 7.66 (s, 1H), 7.54-7.50 (m, 2H), 7.46-7.39 (m, 2H), 7.05 (s, 1H), 6.96 (s, 1H), 4.76-4.75 (d, 2H). MS[M+H]=429; LCMS RT=2.71 min

Compound 35: 400 MHz ¹H NMR (DMSO): 12.56 (s, 1H), 8.66 (s, 1H), 8.39 (s, 1H), 8.34 (s, 2H), 7.71 (s, 1H), 7.29 (d, 1H), 7.04 (s, 1H), 6.95 (s, 1H), 4.74-4.73 (d, 2H). MS[M+H]=419; LCMS RT=2.21 min

Compound 36: 400 MHz ¹H NMR (DMSO): 12.68 (s, 1H), 8.74 (s, 1H), 8.67-8.64 (t, 1H), 8.58 (s, 1H), 7.80 (s, 1H), 7.73 (s, 1H), 7.37-7.35 (d, 1H), 7.02 (s, 1H), 6.97 (s, 1H), 6.94-6.92 (m, 1H), 4.73-4.71 (d, 2H). MS[M+H]=419; LCMS RT=2.23 min

### Example 9

### Preparation of Compounds 39 and 40

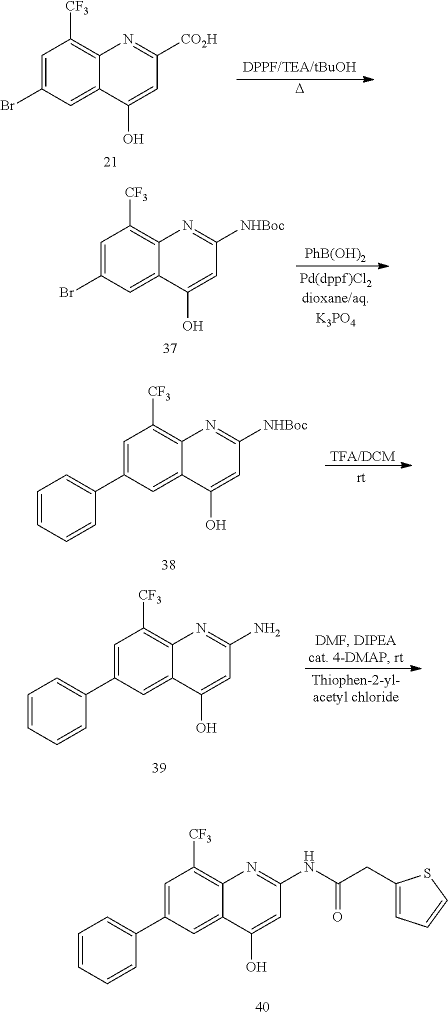

### Step 1

1 g (3 mmol) of compound 21 from example 4 was dissolved in 10 mL t-BuOH and 1.5 mL triethylamine was then added. Finally, 1.1 mL DPPA was added dropwise and the reaction heated to 65° C. under N₂ atmosphere. After overnight, coversion to product was estimated to be 30%. An additional portion of TEA and DPPA were added and the reaction allowed to continue heating for an additional 20 h. At that time, the reaction was diluted with 300 mL EtOAc, washed with water and brine, and concentrated after drying with sodium sulfate. The resulting crude product, which was passed through a short plug of silica gel prior to use in the following procedures, was identified by LC/MS analysis as a mixture of N-Boc carbamate and free aniline, and was utilized directly in the following steps without additional purification.

### Step 2

Standard Suzuki coupling conditions utilizing Pd(dppf)Cl₂, phenyl boronic acid, dioxane/aq. K₃PO₄ were carried out on 300 mg of compound 5003. The resulting crude product was purified by column chromatography (ISCO, 0 to 80% EtOAc in hexanes) to furnish compound 38, 300 mg. ¹H NMR (CDCl₃) diagnostic peaks at δ 8.05 (s, 1H), 7.95 (s, 1H), 1.45 (s, 9H);

MS [M+H]⁺=405.

### Step 3

Biaryl compound 38 was dissolved in 10 mL DCM, and 10 mL TFA was then added. The reaction was monitored by LC/MS and judged complete at t=3 h. After removal of the solvent in vacuo, the product was carried forward without additional purification. An analytical sample was prepared by HPLC purification, furnishing 2 mg of compound 39 as a white powder.

¹H-NMR (DMSO-d₆) δ 10.95 (s, 1H), 8.77 (s, 1H) 8.32 (s, 1H), 8.21 (bs, 1H), 7.85 (m, 2H), 7.54 (m, 2H), 7.42 (m, 1H), 6.04 (s, 1H);

MS [M+H]+=304.

### Step 4

30 mg (0.1 mmol) of compound 39 was taken up in 2 mL DMF, and treated with 5 equiv DIPEA, 5 mg 4-DMAP, and 2-thiophene-2-yl-acetyl chloride (3 equiv, 0.3 mmol). The reaction was monitored by LC/MS and judged complete at t=3 h. At this time, the reaction mixture was introduced directly onto HPLC for purification of the resulting amide. After lyophilization, the 2 mg of the final product 40 was obtained as a light yellow powder.

¹H-NMR (CD₃OD) δ 8.77 (m, 1H), 8.45 (m, 1H), 7.80 (m, 2H), 7.55 (m, 1H), 7.52 (m, 2H), 7.44 (m, 1H), 7.35 (d, J=4.4 Hz, 1H), 7.06 (d, J=4.4 Hz, 1H), 7.00 (m, 1H), 4.10 (s, 2H);

MS [M+H]⁺=428.

### Example 10

### Preparation of Compounds 47-49

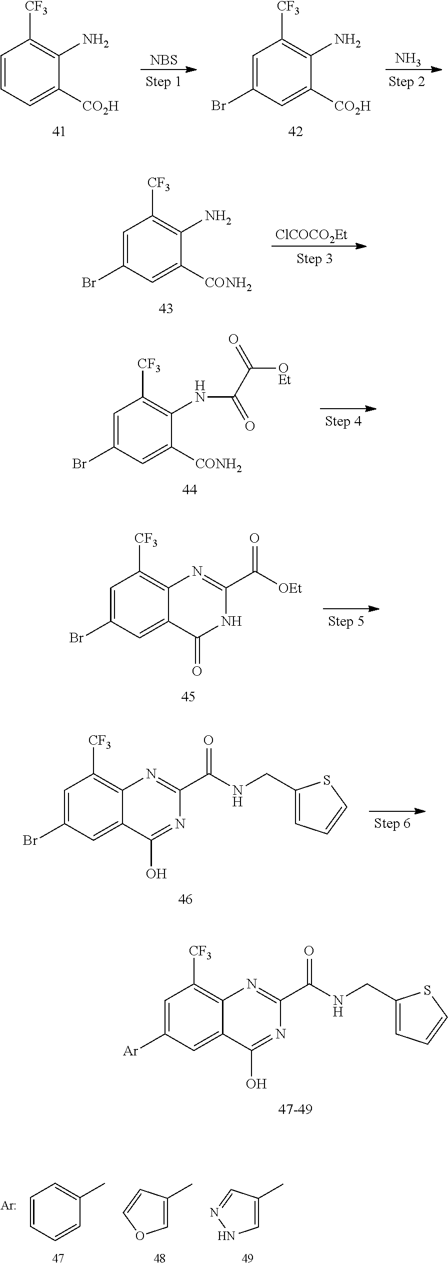

### Step 1

NBS (4.38 g, 24.37 mmol) in DMF (25 mL) was added dropwise to 2-Amino-3-trifluoromethyl-benzoic acid 41 (5 g, 24.37 mmol) dissolved in DMF (25 ml) at RT under N₂. The reaction mixture was stirred at RT for overnight, and it was monitored by LC-MS at negative mood. After completion of the reaction, it was concentrated by reduced pressure evaporation to about 25 mL of solvent left. The mixture was transferred slowly to a beaker containing ˜500 mL ice-water with vigorously stirring. After 30 min, desired product was collected by filtration. The solid was washed with H₂O followed by ether/hexane (1/3) then hexane, and dried under high vacuum to give light yellow solid as compound 42 (6.42 g, 22.6 mmol, 93%). MS [M−H]⁻=282 (98%), 284 (100%).

### Step 2

Acid 42 (6.4 g, 32.53 mmol) was dissolved in 0.5 M NH₃ in dioxane (180 mL, 90.12 mmol). To which, was added EDCl (5.2 g, 27.04 mmol), HOBt (4.0 g, 29.29 mmol), and DIPEA (16.5 mL, 94.63 mmol). The reaction was monitored by LC-MS. After 24 h, it was still about half of the SM (2) left. 100 mL of 0.5M NH3/dioxane was added and the mixture was stirred for another 24 h. It was concentrated, and purified by flash chromatography on silica gel with EA/Hex to give 5.6 g (88%) of compound 43. MS [M+H]⁺=282.9 (100%), 284.9 (99%).

### Step 3

Aniline 43 (2.88 g, 10.2 mmol) with lutidine (2.6 mL, 22.22 mmol) in THF (100 mL) was cooled to 0° C. under N₂. Chloro-ethyloxalate (1.3 mL, 11.22 mmol) was introduced via syringe slowly. The mixture was stirred at 0° C. for 30 min then RT. It was monitored by LC/MS. At 7 h, another 0.6 mL of chloroethyloxalate was added at 0° C. then warmed to RT overnight. Total reaction time was 24 h. MS [M+H]+=381.0 (100%), 383.0 (98%).

### Step 4

Above mixture was heated to 100° C. for 24 h. LC/MS showed little starting material left. After cooled to RT, it was diluted with EA, washed with sat'd NaHCO₃ and brine. The organic layer was dried (Na₂SO₄) and concentrated. The crude product was slurried in EtOAc/Hex (1/1) ((100 mL) for 10 min, stored in freezer overnight. The white solid was collected by filtration and raised with ether/hexane (1/3) then hexane to give 1.45 g (39%) of compound 45. [M+H]+=365.0 (98%), 367.0 (100%).

### Step 5

The mixture of compound 45 (821 mg, 2.25 mmol) and 2-aminomethyl thiophene (0.926 mL, 9.00 mmol) in DMF (22.5 mL) was heated at 90° C. for 2 h. After cooling to RT, the mixture was diluted with water (20 mL), and the pH was adjusted to 6-7 using 1N HCl. The white solid was collected by filtration and rinsed with water, ether/hexane (1/3) and hexane. The solid was air dried to give 1.26 g (still wet) of compound 46. [M+H]+=330.0 (98%), 432.1 (100%).

### Step 6

To a mixture of crude compound 46 (612 mg, 1.416 mmol), phenylboronic acid (264 mg, 2.12 mmol), Pd(PPh₃)₄ (82 mg, 0.07 mmol) in a microwave tube was added dioxane (4.2 ml) and K₃PO₄ (1M) (4.2 ml, 4.2 mmol). The reaction mixture was subjected in microwave at 140° C. for 10 minutes. After cooled to RT, it was diluted with EA, washed sat'd NaHCO₃ and brine. The organic layer was dried (Na₂SO₄) and concentrated. The crude product was slurried in DCM/ether (1/2) ((15 mL) for 10 min. The white solid was collected by filtration and raised with ether/hexane (1/3) then hexane to give 240 mg of compound 47. The mother liquid was further purified by flash chromatography on silica gel with EtOAc/Hex to give 168 mg of compound 47.

¹H-NMR (400 MHz, DMSO-d6) δ 12.81 (sb, 1H), 8.94 (t, 1H), 8.56 (d, 2H), 8.41 (d, 1H), 7.84 (t, 2H), 7.51 (m, 2H), 7.40-7.40 (m, 2H), 7.05 (m, 1H), 6.96 (m, 1H), 4.69 (d, 2H); ¹⁹F NMR (376.1 MHz) δ −58.28 (5); MS [M−H]⁻=428.2.

Compounds 48 and 49 were made by the same procedure as in step 6 using the corresponding boronic acids.

Compound 48: ¹H-NMR (400 MHz, DMSO-d6) δ 12.73 (sb, 1H), 8.90 (t, 1H), 8.53 (d, 2H), 8.39 (s, 1H), 7.80 (m, 1H), 7.40 (m, 1H), 7.05 (s, 1H), 6.95 (m, 1H), 4.67 (d, 2H); ¹⁹F NMR (376.1 MHz) δ −58.28 (s); MS [M−H]⁻=428.2.

Compound 49: ¹H-NMR (400 MHz, DMSO-d6) δ 12.98 (sb, 1H), 8.92 (t, 1H), 8.35 (s, 1H), 8.33 (s, 1H), 8.08 (s, 1H), 7.98 (s, 1H), 7.36 (m, 1H), 7.01 (m, 1H), 6.93 (m, 1H), 4.59 (d, 2H); ¹⁹F NMR (376.1 MHz) δ −58.45 (s); MS [M−H]⁻=418.2.

### Example 11

### Preparation of Compounds 51-53

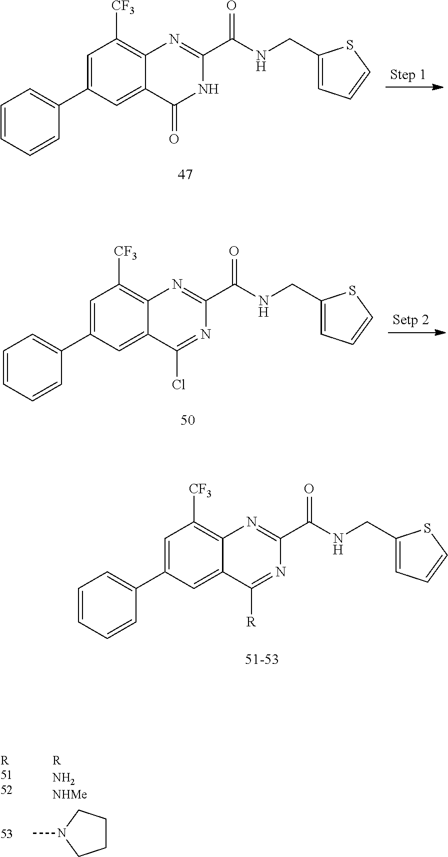

### Step 1

POCl₃ (1.5 mL) was added to 4-oxo-6-phenyl-8-trifluoromethyl-3,4-dihydro-quinazoline-2-carboxylic acid (thiophen-2-ylmethyl)-amide 47 (50 mg, 0.116 mmol) dissolved in dioxane (1.5 ml) at RT under N₂. The reaction mixture was heated to 100° C. for 2 h. It was monitored by LC-MS. After completion of the reaction, it was concentrated by evaporation at reduced pressure to dryness and azotropy with toluene twice to give the crude product 50.

### Step 2

Crude compound 50 was dissolved in 7 N NH₃ in MeOH (5 mL) and stirred at RT for 18 h. The reaction was monitored by LC-MS. It was concentrated, and mixture purified by RP-HPLC to give compound 51 (15 mg, 30%).

¹H-NMR (400 MHz, DMSO-d6) δ 8.87 (s, 1H), 8.78 (t, 1H), 8.51 (br, 1H), 8.43 (s, 1H), 8.37 (br, 1H), 7.87 (d, 2H), 7.53 (t, 2H), 7.42 (d, d, 2H), 7.03 (m, 1H), 6.96 (m, 1H), 4.66 (d, 2H); ¹⁹F NMR (376.1 MHz) δ −58.01 (s); MS [M−H]⁺=429.1.

Compounds 52 and 53 were made by the similar procedure as 51 using the corresponding amines.

Compound 52: ¹H-NMR (400 MHz, DMSO-d6) δ 8.86 (s, 1H), 8.74 (t, 1H), 8.66 (br, 1H), 8.28 (s, 1H), 7.60 (d, 2H), 7.29 (m, 3H), 7.15 (m, 1H), 6.98 (m, 1H), 6.89 (m, 1H), 4.80 (d, 2H), 2.94 (s, 3H); ¹⁹F NMR (376.1 MHz) δ −60.27 (s); MS [M−H]⁺=443.1.

Compound 53: ¹H-NMR (400 MHz, DMSO-d6) δ 8.64 (br, 1H), 8.50 (m, 1H), 8.28 (br, 1H), 7.62 (d, 2H), 7.51 (m, 2H), 7.42 (m, 1H), 7.21 (m, 1H), 7.07 (m, 1H), 6.95 (m, 1H), 4.86 (d, 2H), 4.10 (m, 4H), 2.08 (m, $H); ¹⁹F NMR (376.1 MHz) δ −60.71 (s); MS [M−H]⁺=483.2

### Example 12

### Preparation of Compounds 54

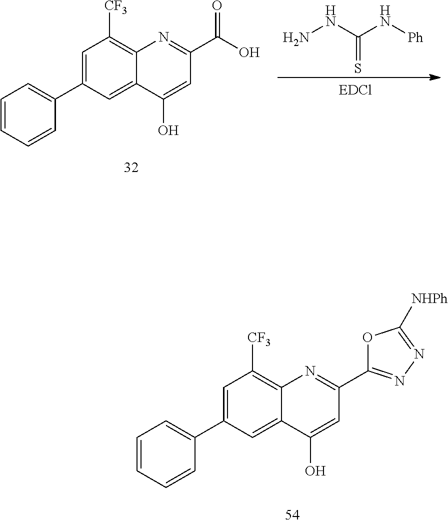

Compound 32 (35 mg, 0.105 mmol), suspended in DCM (2 mL), was treated with 4-phenyl-3-thiosemicarbazide (18 mg, 0.105 mmol) and EDC hydrochloride (60 mg, 0.315 mmol). The yellow-colored suspension was stirred at room temperature overnight. LC/MS showed a mixture of desired product and uncyclized intermediate. Reaction mixture was concentrated. The residue was suspended in DMF and filtered through a syringe filter before purification by prep HPLC to give an off-white solid 54 (5 mg, 12%).

¹H NMR (400 MHz, DMSO-d6) δ 12.39 (s, 1H), 10.89 (s, 1H), 8.59 (s, 1H), 8.38 (s, 1H), 7.84 (d, 2H), 7.68 (m, 3H), 7.52 (m, 2H), 7.48 (m, 1H), 7.40 (m, 2H), 7.01 (t, 1H); ¹⁹F NMR (376.1 MHz) δ −58.52, −73.89 (TFA salt); LC/MS RT=2.60 min.

### Example 13

### Preparation of Compounds 59

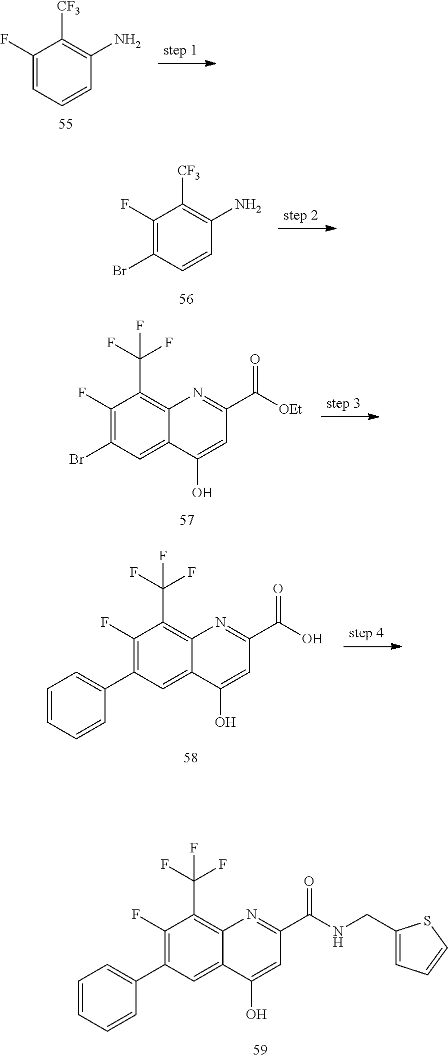

### Step 1

A 100-mL 1-neck rbf was charged with 55 (5.0 g, 27.9 mmol) and DMF (20 mL). The reaction mixture was cooled to 0° C. with ice-water bath. A solution of NBS (5.0 g, 27.9 mmol) in DMF (20 mL) was added dropwise to the reaction mixture with stirring and maintained at 0° C. for 5 minutes. The reaction mixture was EtOAc (200 mL) and washed with 5% LiCl and dried with sodium sulfate. After removal of the solvent in vacuo, intermediate 56 (7.0 g, 98%) was obtained and used for next step without further purification. ¹H-NMR (400 MHz, CCl₃H-d) δ 7.35 (t, J=7.2 Hz, 1H), 6.40 (d, J=9.2 Hz, 1H), 3.83 (br, 2H); ¹⁹F NMR (376.1 MHz, CCl₃H-d) δ −56.05 (d, J=24.4, Hz, 3 F), −105.53 (m, 1 F).

### Step 2

A 250-mL 1-neck rbf was charged with intermediate 56 (7.0 g, 27.1 mmol), diethyl acetylene dicarboxylate (6.2 g, 36.3 mmol) and MeOH (100 mL). The reaction mixture was heated to reflux for overnight. After cooling back to room temperature and removal of the solvent in vacuo, the residue was dissolved in diphenyl ether (20 mL) and heated to 240° C. with stirring for 10 minutes. After the reaction mixture was cooled back to room temperature, the residue was purified by preparative flash chromatography (silica gel, ethyl acetate/hexane gradient) affording 2.5 g of intermediate 57 as a yellow solid. ¹H-NMR (400 MHz, CCl₃H-d) δ 8.70 (d, J=7.2 Hz, 1H), 6.92 (s, 1H), 4.48 (q, J=6.8 Hz, 2H), 1.41 (t, J=6.8 Hz, 3H); ¹⁹F NMR (376.1 MHz, CCl₃H-d) δ −54.74 (d, J=24.4, Hz, 3 F), −94.40 (m, 1 F); MS [M+H]⁺=382.1.

### Step 3

A 25-mL microwave tube was charged with intermediate 57 (0.5 g, 1.31 mmol), phenylboronic acid (0.36 g, 1.91 mmol), tetrakis(triphenylphosphine)palladium(0) (0.075 g, 0.07 mmol), 1M potassium phosphate (3 mL) and dioxane (5 mL). The reaction mixture was heated up to 140° C. under microwave with stirring for 10 mins. The reaction mixture was acidified to pH 2 by adding 1 N HCl and diluted with EtOAc (50 mL) and washed with water (2×50 mL) and dried with sodium sulfate. After removal of the solvent in vacuo, 0.5 g of the intermediate 58 was obtained as a white solid and used for next step without further purification.

### Step 4

A 50-mL 1-neck rbf was charged with intermediate 58 (0.10 g, 0.28 mmol), 2-thiophene methylamine (0.065 g, 0.56 mmol), HATU (0.21 g, 0.56 mmol), NMM (0.14 g, 1.4 mmol) and DMF (3 mL). The reaction mixture was stirred at room temperature for overnight and purified by HPLC to afford compound 59 (65 mg, 50%) as a white solid.

¹H-NMR (400 MHz, CCl₃H-d) δ 10.60 (br, 1H), 8.72 (br, 1H), 8.50 (d, J=7.2 Hz, 1H), 8.22 (s, 1H), 7.50-7.38 (m, 6H), 7.08 (m, 1H), 6.98 (m, 1H), 6.79 (m, 1H), 4.79 (d, J=5.2 Hz, 2H); ¹⁹F NMR (376.1 MHz, CCl₃H-d) δ −54.13 (d, J=23.7 Hz, 3 F), −103.60 (m, 1 F); MS [M+H]⁺=446.4.

### Example 13

### Preparation of Compounds 64

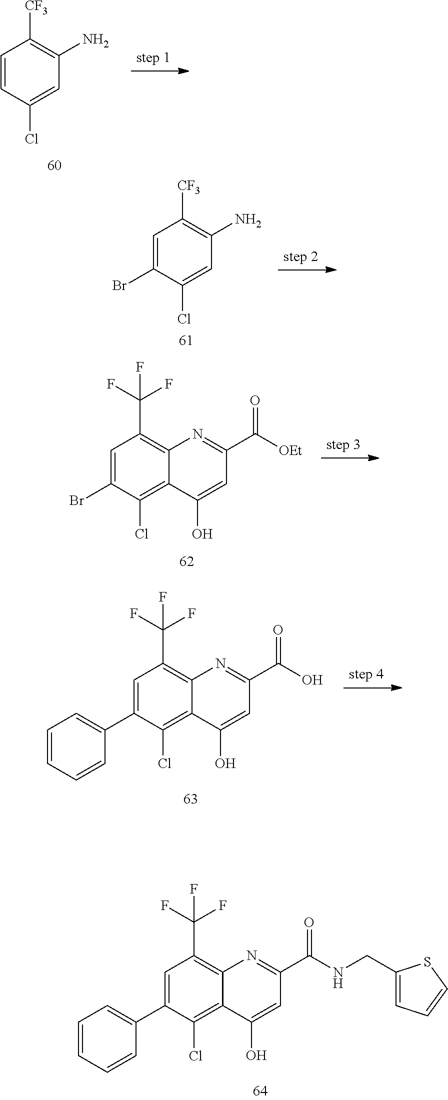

Compound 64 was prepared in a manner similar to that described in preparation of compound 59, except the 5-chloro-2-trifluoromethyl-phenylamine was used in place of the 3-fluoro-2-trifluoromethyl-phenylamine.

¹H-NMR (400 MHz, CCl₃H-d) δ 9.20 (br, 1H), 8.60 (br, 1H), 7.97 (m, 1H), 7.51-7.40 (m, 6H), 7.22 (m, 1H), 7.08 (m, 1H), 6.96 (m, 1H), 4.88 (m, 2H); ¹⁹F NMR (376.1 MHz, CCl₃H-d) δ −60.55 (s); MS [M+H]⁺=462.9.

### Example 14

### Preparation of Compound 65 and 66

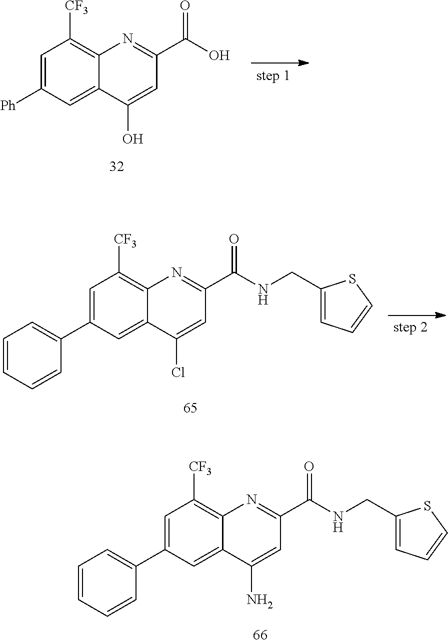

### Step 1

To a mixture of the acid 32 (1.033 g, 3.10 mmol) in thionyl chloride (15 mL) was added ˜4 drops of DMF and refluxed for 20 h. After the solution was concentrated, the residue was azeotrophed with toluene (×2). The resulting residue was stirred with DMF (3 mL) at 0° C. as 2-aminomethylthiophene (0.39 mL, 3.80 mmol) and N-methylmorpholine (1.02 mL, 9.27 mmol) were added. The mixture was diluted with DMF (2 mL), and stirred at it for 1 h, diluted with water (˜25 mL) and ethyl acetate (˜15 mL). Stirring was continued at 0° C. for 30 min and filtered. The solids collected were washed with water and ethyl acetate and dried in vacuum to afford amide 65 (700 mg, 51%). ¹H-NMR (400 MHz, DMSO-d₆) δ 8.84 (t, J=6.0 Hz, 1H), 8.67 (d, J=1.6 Hz, 1H), 8.60 (br s, 1H), 8.41 (s, 1H), 7.95 (d, J=6.0 Hz, 2H), 7.59 (t, J=7.2 Hz, 2H), 7.52 (t, J=7.2 Hz, 1H), 7.43 (dd, J=5.2 and 1.2 Hz, 1H), 7.11 (br d, J=2.4 Hz, 1H), 7.00 (dd, J=5.2 and 3.2 Hz, 1H), 4.81 (d, J=6.0 Hz, 2H); ¹⁹F NMR (376.1 MHz, DMSO-d₆) δ− 58.32 (s); MS [M+H]⁺=446.9 (100%), 448.8 (40%)

### Step 2

The vials containing a mixture of compound 65 (25 mg, 0.056 mmol) in concentrated ammonium hydroxide (20 mL) was heated at 150° C. for 1 h in a microwave reactor. After a lump of starting material-like solids were removed from each vial, the two suspensions were combined, concentrated, and dried in vacuum. The residue was dissolved in DMF with a drop of trifluoroacetic acid, filtered, and purified by preparative HPLC to obtain compound 66 (11 mg, 19%).

¹H-NMR (400 MHz, CD₃OD) δ 8.67 (s, 1H), 8.35 (s, 1H), 7.81 (d, J=7.2 Hz, 1H), 7.53 (t, J=7.2 Hz, 2H), 7.41-7.44 (m, 2H), 7.33 (dd, J=5.2 and 1.2 Hz, 1H), 7.10 (br d, J=2.1 Hz, 1H), 6.99 (dd, J=5.2 and 3.6 Hz, 1H), 4.84 (s, 2H); ¹⁹F NMR (376.1 MHz, CD₃OD) δ −61.64 (s, 3H), −77.51 (s, 3H); MS [M+H]⁺=428.0

### Example 15

### Preparation of Compound 68

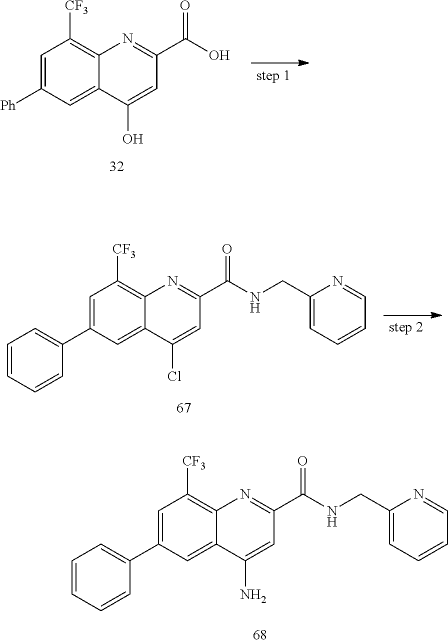

### Step 1

Compound 67 (40 mg, 25%) was prepared in a manner similar to that described in the synthesis of compound 65, except 2-aminomethylpyridine was used in place of 2-aminomethylthiophene. ¹H-NMR (400 MHz, DMSO-d₆) δ 9.22 (t, J=5.2 Hz, 1H), 8.70 (br d, J=1.6 Hz, 1H), 8.64 (br s, 1H), 8.58 (d, J=4.8 Hz, 1H), 8.43 (s, 1H), 7.97 (d, J=7.6 Hz, 2H), 7.81 (td, J=7.8 and 1.6 Hz, 1H), 7.60 (t, J=7.4 Hz, 2H), 7.54 (t, J=7.2 Hz, 1H), 7.43 (d, J=8.4 Hz, 1H), 7.33 (dd, J=7.2 and 5.2 Hz, 1H), 4.76 (d, J=5.2 Hz, 2H); ¹⁹F NMR (376.1 MHz, DMSO-d₆) δ −58.43 (s); MS [M+H]⁺=442.1 (100%), 444.1 (35%)

### Step 2

A mixture of compound 67 (31 mg, 0.070 mmol) in concentrated ammonium hydroxide (20 mL) was heated at 150° C. for 1 h at microwave reactor. After the suspension was concentrated, and dried in vacuum, the residue was dissolved in DMF with a drop of trifluoroacetic acid, filtered, and purified by preparative HPLC to obtain compound 68 (17 mg, 37%).

¹H-NMR (400 MHz, CD₃OD) δ 8.71 (br d, J=2.4 Hz, 1H), 8.64 (d, J=2.0 Hz, 1H), 8.39 (td, J=7.2 and 1.6 Hz, 1H), 8.33 (s, 1H), 7.92 (d, J=8.4 Hz, 1H), 7.77-7.84 (m, 3H), 7.52 (t, J=7.6 Hz, 2H), 7.40-7.47 (m, 2H), 4.97 (s, 2H); ¹⁹F NMR (376.1 MHz, CD₃OD) δ −61.43 (s, 3H), −77.26 (s, 6H); MS [M+H]⁺=423.2

### Example 16

### Preparation of Compound 70

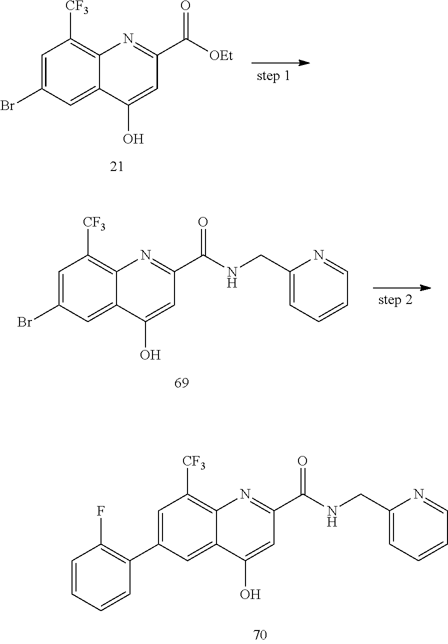

### Step 1

The crude acid 21 and HATU (3.146 g, 8.28 mmol) in DMF (20 mL) was stirred at rt as 2-aminomethylpyridine (0.68 mL, 6.65 mmol) and N-methylmorpholine (2.2 mL, 20.00 mmol) were added. After 2 h, the mixture was diluted with 5% aqueous LiCl (˜120 mL) and ethyl acetate (˜400 mL) and heated to warm, and filtered to remove remaining solids. Two phases of the filtrate were separated and the aqueous fraction was extracted with warm ethyl acetate (˜200 mL). After the organic fractions were washed with warm water (×2), combined, dried (MgSO₄), and concentrated. The residue was triturated with ethyl acetate (20˜30 mL) at rt for 5 min, and filtered. The solids collected were washed with ethyl acetate, and dried in vacuum to afford compound 69 (1.36 g, 58%). MS [M+H]⁺=426.1 and 428.1

Compound 70 (45 mg, 57%) was prepared from 69 in a manner similar to that described in the synthesis of compound 63, except 2-fluorophenylboronic acid was used in place of phenylboronic acid.

¹H-NMR (400 MHz, CD₃OD) δ 8.71 (br d, J=5.2 Hz, 1H), 8.67 (s, 1H), 8.40 (td, J=8.0 and 1.6 Hz, 1H), 8.33 (s, 1H), 7.95 (d, J=8.0 Hz, 1H), 7.82 (t, J=6.4 Hz, 1H), 7.52-7.69 (m, 3H), 7.46-7.52 (m, 1H), 7.36 (td, J=7.6 and 1.2 Hz, 1H), 7.26-7.33 (m, 1H), 5.00 (s, 2H), 2.31 (s, 3H); ¹⁹F NMR (376.1 MHz, CD₃OD) δ −61.43 (s, 3H), −77.45 (s, 6H), −120.25 (m, 1H); MS [M+H]⁺=442.2

### Example 17

### Preparation of Compound 79

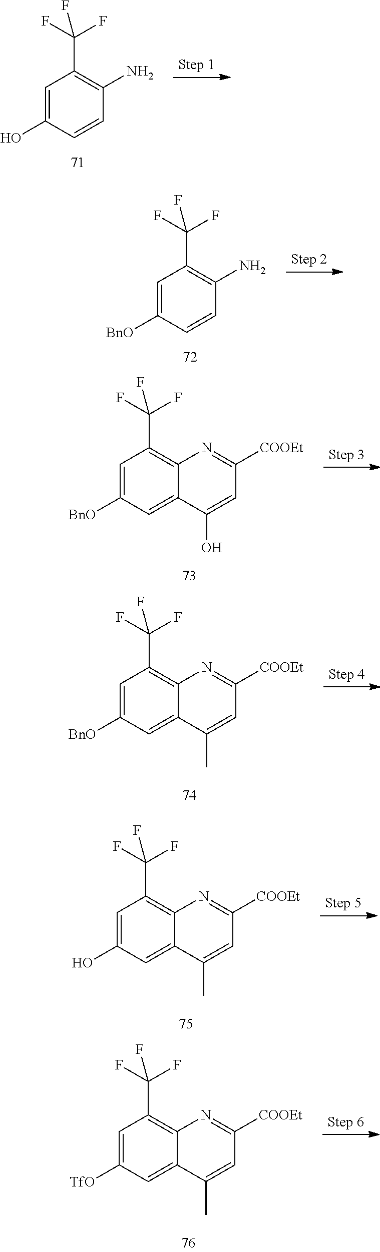

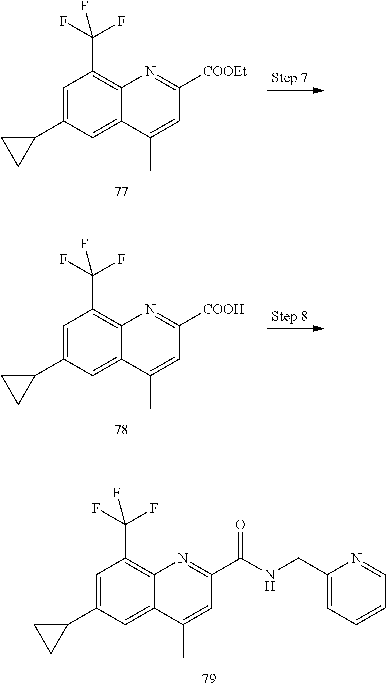

### Step 1

A solution of 4-amino-2-(trifluoromethyl)phenol (compound 71, 4.302 g, 24.3 mmol), triphenylphosphine (7.650 g, 29.2 mmol), and benzyl alcohol (3.05 mL, 29.5 mmol) in THF (50 mL) was stirred at 0° C. as diisopropyl azodicarboxylate (5.65 mL, 29.2 mmol) was added. After 5 min at 0° C., the ice bath was removed and the resulting solution was stirred at rt for 18 h. After the solution was concentrated, the residue was triturated with ethyl acetate (˜30 mL) before filtering insoluble triphenylphosphine oxide. The filtrate was concentrated and the residue was purified by combiflash using hexane-ethyl acetate to obtain 5.854 g (90%) of compound 72. MS [M+H]⁺=268.0

### Step 2

A solution of the compound 72 (5.854 g, 21.9 mmol) and diethylacetylenedicarboxylate (3.85 mL, 24.2 mmol) in methanol (22 mL) was refluxed for 3 h. The solution was concentrated and the resulting viscous syrup was dried in vacuum for ˜30 min. The crude product was used for the cyclization.

A solution of crude adduct in diphenyl ether (22 mL) was heated with a heating mantle (set at 250° C.) while monitoring the inner temperature. At 15 min, the inner temperature reached to 200° C. At 25 min, the inner temperature became ˜220° C. and the mixture boiled due to ethanol formed. After 30 min, the black solution was cooled to it and then diluted with hexanes (˜30 mL). The resulting mixture was stirred at it for ˜30 min and the solids formed were filtered. After the solids were washed with a mixture (˜2:3) of diphenyl ether and hexanes followed by hexanes, it was dried to get 6.067 g (71%) of compound 73 containing a little bit of diphenyl ether. MS [M+H]⁺=392.1

### Step 3

A solution of compound 73 (5.645 g, 14.4 mmol), 4-dimethylaminopyridine (175 mg, 1.43 mmol), and 2,6-lutidine (5.0 mL, 43.1 mmol) in dichloromethane (50 mL) was stirred at 0° C. as trifluoromethanesulfonyl chloride (3.8 mL, 35.9 mmol) was added dropwise. After 5 min, the ice bath was removed and the solution was stirred at it for 1 h. The solution was concentrated and the residue was dissolved in ethyl acetate (˜170 mL) and ice cold 0.2 N HCl (˜250 mL). After the two phases were separated, the aqueous fraction was extracted with ethyl acetate (˜50 mL×1). The organic fractions were washed with ice cold water (×1), combined, dried (Na₂SO₄), and concentrated. The crude triflate was used for the next reaction.

A mixture of the crude triflate, Pd(dppf)Cl₂—CH₂Cl₂ (1.175 g, 1.44 mmol), methylboronic acid (2.592 g, 43.3 mmol), and powdered K₂CO₃ (7.971 g, 57.68 mmol) in 1,4-dioxane (50 mL) was refluxed at 105° C. bath for 2 h. The mixture was diluted with ethyl acetate (˜250 mL) and washed with water (˜250 mL×2). The aqueous fractions were extracted with ethyl acetate (250 mL×1), and the combined organic fractions were dried (Na₂SO₄), and concentrated with silicagel. The adsorbed product was purified by combiflash hexanes-ethyl acetate to obtain 5.178 g (92%) of compound 74. MS [M+H]⁺=390.1

### Step 4

A mixture of compound 74 (5.168 g, 13.27 mmol) and 10% Pd/C (511 mg) in methanol (40 mL) and ethyl acetate (80 mL) was stirred vigorously under H₂ atmosphere. After 1.5 h, additional 10% Pd/C (517 mg) was added and the resulting mixture was stirred under H₂ atmosphere for 6 h. The mixture was filtered through celite pad and the filterate was concentrated to obtain 3.945 g (99%) of compound 75. MS [M+H]⁺=300.0

### Step 5

A solution of compound 75 (3.643 g, 12.17 mmol), DMAP (149 mg, 1.22 mmol), and 2,6-lutidine (6.4 mL, 55.1 mmol) in dichloromethane (50 mL) was stirred at 0° C. as trifluoromethanesulfonyl chloride (4.9 mL, 46.3 mmol) was added After 5 min, the solution was warmed to rt and stirred for 1.5 h. The solution was concentrated and the residue was dissolved in ethyl acetate (˜150 mL) before washing with ice-cold 0.5 N HCl (×1). The separated aqueous solution was extracted with ethyl acetate (100 mL×1). The organic fractions were washed with water (×1), combined, dried (Na₂SO₄), and concentrated to obtain crude compound 28. The crude compound 76 was used for the next reaction. MS [M+H]⁺=432.0

### Step 6

A mixture of compound 76 (954 mg, 2.21 mmol), Pd(dppf)Cl₂—Ch₂Cl₂ (181 mg, 0.221 mmol), cyclopropylboronic acid (570 mg, 6.64 mmol), and powdered K₂CO₃ (1.23 g, 8.90 mmol) in dioxane (19 mL) was refluxed at 105° C. bath for 2 h before additional cyclopropylboronic acid (190 mg, 2.21 mmol), and powdered K₂CO₃ (260 mg, 1.88 mmol) were added. The mixture was refluxed for 3 h more and diluted with water (˜150 mL) before extraction with ethyl acetate (˜100 mL×2). The extracts were washed with water (×1), combined, dried (Na₂SO₄), and concentrated. The residue was purified by combiflash (80 g column) using hexane and ethyl acetate as eluents to obtain 604 mg (84%) of compound 77. MS [M+H]⁺=324.1

### Step 7

A solution of compound 77 (604 mg, 1.87 mmol) in THF (12 mL), methanol (12 mL), and 1 N KOH (5.6 mL, 5.6 mmol) was stirred at it for 1 h. After the solution was acidified with 1 N HCl (6 mL), the mixture was concentrated to ˜1/3 volume, diluted with water, and extracted with ethyl acetate (×2). The extracts were washed with water (×1), combined, dried (Na₂SO₄), and concentrated to obtain crude compound 78. The crude compound 30 was used for the next reaction. MS [M+H]⁺=296.0

### Step 8

Compound 79 (342 mg, 90%) was prepared from compound 78 (293 mg, 0.992 mmol) in a manner that described previously. ¹H-NMR (400 MHz, CDCl₃) δ 9.15 (br t, 1H), 8.64 (d, J=4.4 Hz, 1H), 8.22 (s, 1H), 7.90 (s, 1H), 7.78 (s, 1H), 7.71 (br, 1H), 7.40 (br d, J=6.4 Hz, 1H), 7.24 (br, 1H), 4.89 (br d, 2H), 2.79 (s, 3H), 2.18 (m, 1H), 1.17 (m, 2H), 0.90 (m, 2H); ¹⁹F NMR (376.1 MHz, CDCl₃) δ −59.89 (s, 3 F); MS [M+H]⁺=386.2

The following compounds were prepared in similar manners:

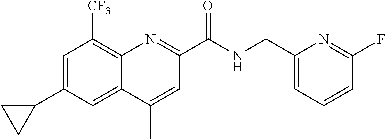

¹H-NMR (400 MHz, CDCl3) δ 8.93 (t, 1H), 8.18 (s, 1H), 7.89 (s, 1H), 7.74 (m, 2H), 7.23 (d, 1H), 6.83 (d, 1H), 4.79 (d, 2H), 2.77 (s, 3H), 2.16 (m, 1H), 1.15 (m, 2H), 0.88 (m, 2H); ¹⁹F NMR (376.1 MHz) δ −60.39 (s), −67.84 (d); MS [M−H]⁺=404.17.

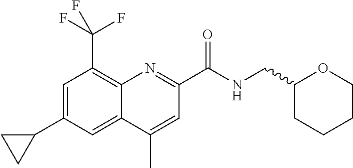

¹H-NMR (400 MHz, CDCl₃) δ 8.63 (br t, 1H), 8.16 (s, 1H), 7.87 (s, 1H), 7.75 (s, 1H), 4.06 (m, 1H), 3.71 (ddd, J=13.6, 6.8, and 4.0 Hz, 1H), 3.44-3.58 (m, 2H), 3.38 (ddd, J=13.2, 7.6, and 4.8 Hz, 1H), 2.76 (s, 3H), 2.16 (m, 1H), 1.87 (br d, J=˜10.4 Hz, 1H), 1.35-1.69 (m, 5H), 1.16 (m, 2H), 0.88 (m, 2H); ¹⁹F NMR (376.1 MHz, CDCl₃) δ −60.01 (s, 3 F); MS [M+H]⁺=393.2

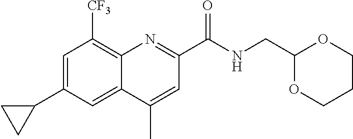

¹H-NMR (400 MHz, CH₃OH-d₄) δ 8.12 (s, 1H), 8.07 (s, 1H), 7.88 (s, 1H), 4.18 (m, 2H), 3.88 (m, 2H), 3.62 (m, 2H), 2.82 (s, 3H), 2.25 (m, 1H), 2.15 (m, 1H), 1.44 (m, 2H), 1.21 (m, 2H), 0.95 (m, 2H); ¹⁹F NMR (400 MHz, CH₃OH-d₄) δ −61.81 (s); MS [M+H]⁺=395.

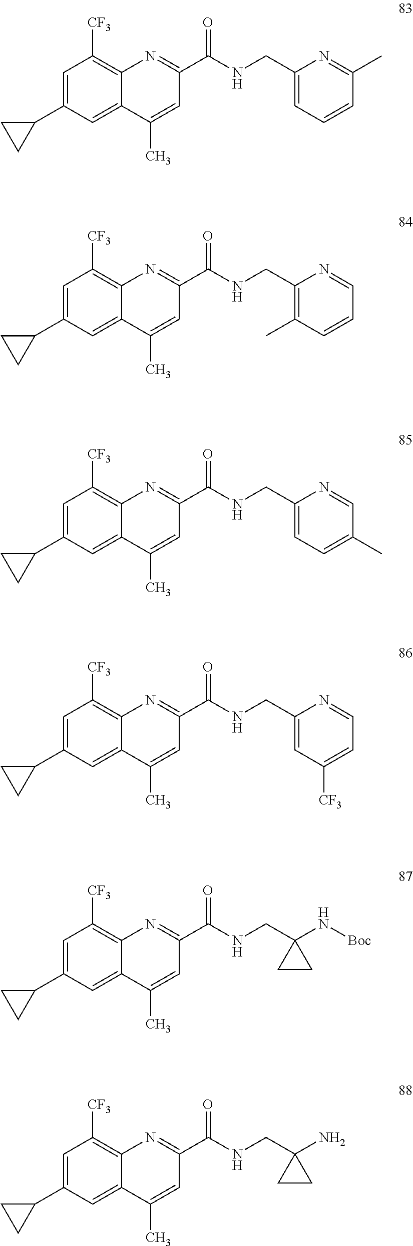

Compound 83: ¹H-NMR (400 MHz, CDCl₃) δ 9.36 (bs, 1H), 8.20 (m, 1H), 7.87 (m, 1H), 7.76 (m, 1H), 7.54 (m, 1H), 7.12 (m, 1H), 7.06 (m, 1H), 4.79 (d, 2H), 2.76 (s, 3H), 2.61 (s, 3H), 2.13 (m, 1H), 1.15 (m, 2H), 0.87 (m, 2H). ¹⁹F NMR (376.1 MHz) δ −60.20 (s); MS [M+H]⁺=400.17.

Compound 84: ¹H-NMR (400 MHz, CDCl₃) δ 8.52 (m, 1H), 8.19 (s, 1H), 7.87 (s, 1H), 7.76 (s, 1H), 7.50 (m, 1H), 7.17 (m, 1H), 4.76 (d, 2H), 2.76 (s, 3H), 2.36 (s, 3H), 2.13 (m, 1H), 1.15 (m, 2H), 0.87 (m, 2H). ¹⁹F NMR (376.1 MHz) δ −60.43 (s); MS [M+H]⁺=400.19.

Compound 85: ¹H-NMR (400 MHz, CDCl₃) δ 9.09 (bs, 1H), 8.43 (m, 1H), 8.19 (s, 1H), 7.87 (m, 1H), 7.75 (m, 1H), 7.45 (m, 1H), 7.25 (m, 1H), 4.79 (d, 2H), 2.76 (s, 3H), 2.31 (s, 3H), 2.13 (m, 1H), 1.15 (m, 2H), 0.88 (m, 2H). ¹⁹F NMR (376.1 MHz) δ −60.37 (s); MS [M+H]⁺=400.18.

Compound 86: ¹H-NMR (400 MHz, CDCl₃) δ 9.12 (m, 1H), 8.79 (m, 1H), 8.19 (s, 1H), 7.88 (m, 1H), 7.76 (m, 1H), 7.56 (m, 1H), 7.42 (m, 1H), 4.92 (d, 2H), 2.76 (s, 3H), 2.15 (m, 1H), 1.15 (m, 2H), 0.88 (m, 2H). ¹⁹F NMR (376.1 MHz) δ −60.41 (s), −65.35 (s); MS [M+H]⁺=454.16.

Compound 87: ¹H-NMR (400 MHz, CDCl₃) δ 8.60 (bs, 1H), 8.15 (s, 1H), 7.85 (s, 1H), 7.75 (s, 1H), 5.00 (bs, 1H), 3.66 (d, 2H), 2.75 (s, 3H), 2.16 (m, 1H), 1.43 (s, 9H), 1.15 (m, 2H), 0.87 (m, 2H). ¹⁹F NMR (376.1 MHz) δ −60.35 (s); MS [M+H]⁺=463.92.

Compound 88: ¹H-NMR (400 MHz, CDCl₃) δ 8.73 (bs, 1H), 8.10 (s, 1H), 8.03 (s, 1H), 7.86 (s, 1H), 3.76 (d, 2H), 2.73 (s, 3H), 2.24 (m, 1H), 1.20-0.93 (m, 8H). ¹⁹F NMR (376.1 MHz) δ −61.54 (s), 177.52 (s, TFA); MS [M+H]⁺=364.38.

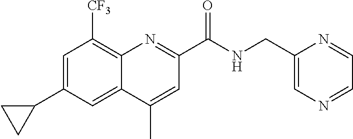

¹H-NMR (400 MHz, CH₃OH-d₄) δ 8.71 (s, 1H), 8.63 (m, 1H), 8.58 (m, 1H), 8.17 (s, 1H), 8.11 (s, 1H), 8.03 (s, 1H), 4.61 (s, 2H), 2.81 (s, 3H), 2.25 (m, 1H), 1.20 (m, 2H), 0.95 (m, 2H); ¹⁹F NMR (400 MHz, CH₃OH-d₄) δ −61.64 (s); MS [M+H]⁺=387.

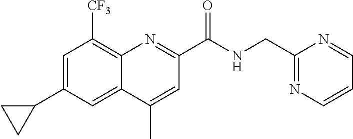

¹H-NMR (400 MHz, CH₃OH-d₄) δ 8.80 (m, 2H), 8.17 (s, 1H), 8.09 (s, 1H), 7.88 (s, 1H), 7.43 (m, 1H), 4.61 (s, 2H), 2.81 (s, 3H), 2.25 (m, 1H), 1.20 (m, 2H), 0.95 (m, 2H); ¹⁹F NMR (400 MHz, CH₃OH-d₄) δ −61.34 (s); MS [M+H]⁺=387.

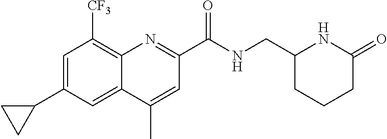

¹H-NMR (400 MHz, CH₃OH-d₄) δ 8.09 (s, 1H), 8.05 (s, 1H), 7.86 (s, 1H), 3.69 (m, 2H), 3.54 (m, 1H), 2.80 (s, 3H), 2.29 (m, 3H), 1.99 (m, 2H), 1.76 (m, 1H), 1.60 (m, 1H), 1.18 (m, 2H), 0.93 (m, 2H); ¹⁹F NMR (400 MHz, CH₃OH-d₄) δ −61.64 (s); MS [M+H]⁺=406.

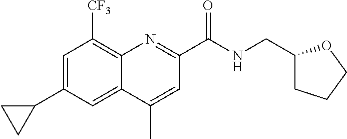

¹H-NMR (400 MHz, CH₃OH-d₄) δ 8.12 (s, 1H), 8.07 (s, 1H), 7.88 (s, 1H), 4.18 (m, 1H), 4.00 (m, 1H), 3.88 (m, 1H), 3.62 (m, 2H), 2.82 (s, 3H), 2.25 (m, 1H), 2.13-1.92 (m, 3H), 1.72 (m, 1H), 1.21 (m, 2H), 0.95 (m, 2H); ¹⁹F NMR (400 MHz, CH₃OH-d₄) δ −61.61 (s); MS [M+H]⁺=379.

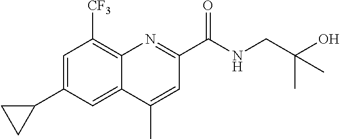

¹H-NMR (400 MHz, CH₃OH-d₄) δ 8.08 (s, 1H), 8.04 (s, 1H), 7.85 (s, 1H), 3.41 (s, 2H), 2.82 (s, 3H), 2.25 (m, 1H), 1.33 (s, 6H), 1.18 (m, 2H), 0.92 (m, 2H); ¹⁹F NMR (400 MHz, CH₃OH-d₄) δ −61.65 (s); MS [M+H]⁺=367.

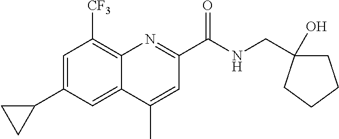

¹H-NMR (400 MHz, CH₃OH-d₄) δ 8.11 (s, 1H), 8.07 (s, 1H), 7.88 (s, 1H), 3.59 (s, 2H), 2.81 (s, 3H), 2.25 (m, 1H), 1.83-1.62 (m, 8H), 1.18 (m, 2H), 0.92 (m, 2H); ¹⁹F NMR (400 MHz, CH₃OH-d₄) δ −61.41 (s); MS [M+H]⁺=393.

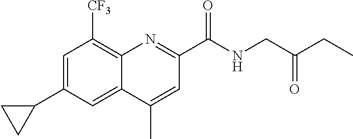

¹H-NMR (400 MHz, CH₃OH-d₄) δ 8.09 (s, 1H), 8.04 (s, 1H), 7.85 (s, 1H), 4.39 (s, 2H), 2.81 (s, 3H), 2.60 (m, 2H), 2.25 (m, 1H), 1.19 (m, 2H), 1.12 (m, 3H), 0.92 (m, 2H); ¹⁹F NMR (400 MHz, CH₃OH-d₄) δ −61.64 (s); MS [M+H]⁺=365.

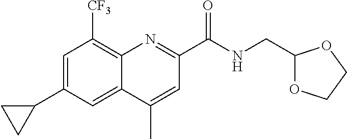

¹H-NMR (400 MHz, CH₃OH-d₄) δ 8.10 (s, 1H), 8.04 (s, 1H), 7.83 (s, 1H), 5.11 (m, 1H), 4.02 (m, 2H), 3.93 (m, 2H), 3.69 (m, 2H), 2.80 (s, 3H), 2.27 (m, 1H), 1.18 (m, 2H), 0.95 (m, 2H); ¹⁹F NMR (400 MHz, CH₃OH-d₄) δ −61.39 (s); MS [M+H]⁺=381.

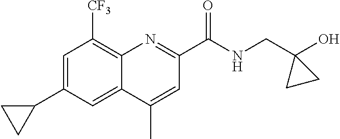

¹H-NMR (400 MHz, CH₃OH-d₄) δ 8.08 (s, 1H), 8.01 (s, 1H), 7.82 (s, 1H), 3.61 (s, 2H), 2.79 (s, 3H), 2.22 (m, 1H), 1.13 (m, 2H), 0.91 (m, 2H), 0.88 (m, 2H), 0.70 (m, 2H); ¹⁹F NMR (400 MHz, CH₃OH-d₄) δ −61.66 (s); MS [M+H]⁺=365.

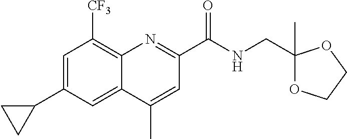

¹H-NMR (400 MHz, CH₃OH-d₄) δ 8.10 (s, 1H), 8.04 (s, 1H), 7.83 (s, 1H), 4.01 (s, 3H), 3.65 (m, 2H), 2.81 (s, 3H), 2.25 (m, 1H), 1.18 (m, 2H), 0.93 (m, 2H); ¹⁹F NMR (400 MHz, CH₃OH-d₄) δ −61.75 (s); MS [M+H]⁺=395.

### Preparation of Compounds 100 to 101

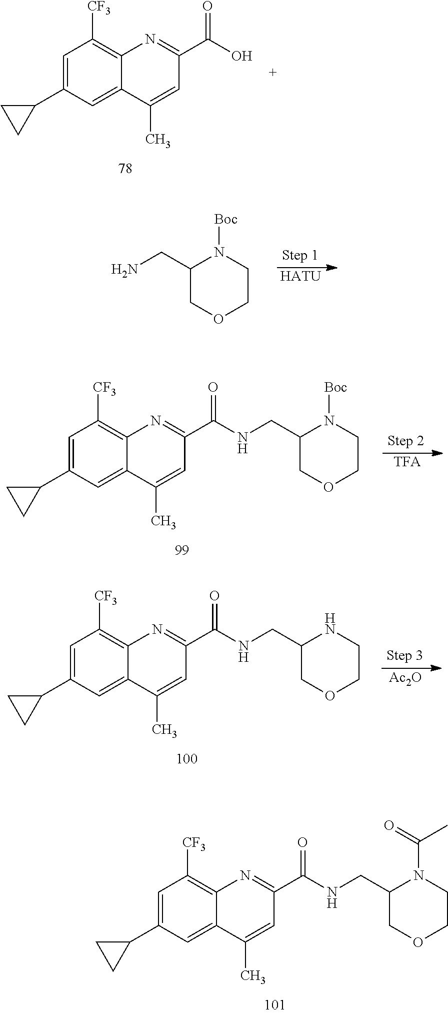

### Step 1

These compounds were made according to procedures in example compound 79.

### Compound 99

¹H-NMR (400 MHz, CD₃OD) δ 8.49 (bs, 1H), 8.06 (s, 1H), 7.99 (s, 1H), 7.82 (s, 1H), 4.23 (m, 1H), 4.13 (m, 1H), 3.89-3.72 (m, 3H), 3.57 (m, 2H), 3.40 (m, 2H), 2.75 (s, 3H), 2.21 (m, 1H), 1.15 (m, 2H), 1.10 (s, 9H), 0.90 (m, 2H). ¹⁹F NMR (376.1 MHz) δ −61.29 (s); MS [M+H]⁺=493.80.

### Compound 100

Compound 100 was from compound 99 by treating with TFA.

¹H-NMR (400 MHz, CD₃OD) δ 8.49 (bs, 1H), 8.14 (m, 1H), 8.07 (m, 1H), 7.87 (m, 1H), 4.01 (m, 2H), 3.85-3.57 (m, 5H), 3.35-3.15 (m, 2H), 2.26 (m, 1H), 1.15 (m, 2H), 0.90 (m, 2H). ¹⁹F NMR (376.1 MHz) δ −61.53 (s), −77.47 (s, TFA); MS [M+H]⁺=394.02.

### Compound 101

Compound 100 (60 mg, 0.122 mmol) dissolved in Py (1 ml) was added Ac₂O (0.3 mL). The reaction was stirred at RT for 1 h for completion. Reaction mixture was diluted with EtOAc, washed with sat'd NaHCO₃ and brine. The organic layer was dried (Na₂SO₄) and concentrated. The crude product was purified by flash chromatography on silica gel with EA/Hex to give 53 mg (100%) of compound 101.

¹H-NMR (400 MHz, CD₃OD) δ 8.49 (bs, 1H), 8.07 (m, 1H), 8.04 (m, 1H), 7.85 (m, 1H), 4.75 (m, 1H), 4.30-4.11 (m, 2H), 3.93 (m, 2H), 3.72-3.40 (m, 4H), 2.79 (s, 3H), 2.26 (m, 1H), 2.06 & 1.99 (s, s, 3H), 1.15 (m, 2H), 0.90 (m, 2H). ¹⁹F NMR (376.1 MHz) δ −61.47, 61.49 (s, s); MS [M+H]⁺=436.09.

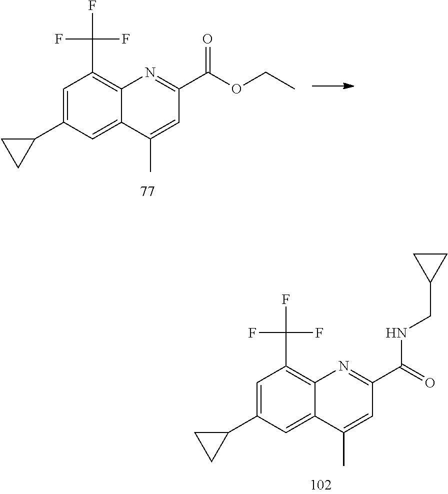

Compound 77 (100.2 mg, 0.31 mmol), aminomethylcyclopropylamine (91.4 mg, 1.28 mmol) and DMF (2 mL) were heated in a microwave reactor (100° C., 15 min; 120° C., 15 min; 140° C., 15 min; 160° C., 15 min). An additional portion of amine (77.2 mg, 1.08 mmol) was added and heating was continued (180° C., 30 min; 180° C., 1 h; 180° C., 1 h) before adding additional amine (200 μL) and continuing to heat (200° C., 1 h). The reaction was concentrated, portioned between ethyl acetate and 5% aqueous LiCl, washing of the organic phase with water and brine before drying (Na₂SO₄) and concentrating again. Purification was accomplished via flash chromatography (silica gel), affording 65.2 mg of compound 102.

¹H NMR (400 MHz, dmso) δ 2.23 (dt, J=3.7, 1.8 Hz, 1H), δ 8.32 (t, J=5.8 Hz, 1H), 8.17-8.11 (m, 2H), 7.98 (d, J=1.6 Hz, 1H), δ 3.38-3.29 (m, 1H), 2.85 (d, J=0.7 Hz, 3H), 2.36 (dq, J=8.3, 5.0 Hz, 1H), 1.27-1.06 (m, 3H), 1.06-0.96 (m, 2H), 0.63-0.40 (m, 2H), 0.42-0.2 (m, 2H); ¹⁹F NMR (376 MHz, dmso) δ −58.89 (s), MS [M+H]⁺=349.02

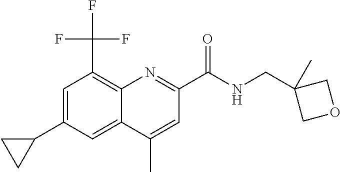

¹H NMR (400 MHz, dmso) δ 8.46 (t, J=6.4 Hz, 1H), 8.08 (dd, J=4.5, 1.3 Hz, 2H), 7.91 (d, J=1.6 Hz, 1H), 5.72 (s, 1H), 4.40 (d, J=5.9 Hz, 2H), 4.22 (d, J=5.9 Hz, 2H), 3.58 (d, J=6.5 Hz, 2H), 2.78 (d, J=0.8 Hz, 3H), 2.35-2.21 (m, 1H), 1.27 (s, 3H), 1.11 (ddd, J=8.3, 6.7, 4.3 Hz, 2H), 0.96 (dt, J=6.8, 4.6 Hz, 2H). ¹⁹F NMR (376 MHz, dmso) δ −58.97 (s), MS [M+H]⁺=379.08.

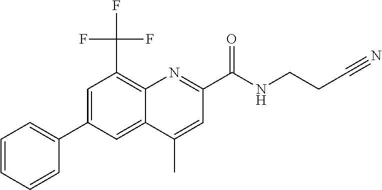

¹H NMR (400 MHz, dmso) δ 8.63 (s, 1H), 8.56 (t, J=6.2 Hz, 1H), 8.48 (s, 1H), 8.17 (s, 1H), 7.95 (d, J=7.3 Hz, 2H), 7.55 (t, J=7.5 Hz, 2H), 7.47 (t, J=7.3 Hz, 1H), 3.67 (q, J=6.5 Hz, 2H), 2.90 (s, 3H) 2.84 (t, J=6.5 Hz, 2H); ¹⁹F NMR (376 MHz, dmso) δ −58.25 (s); MS [M+H]⁺=384.08.

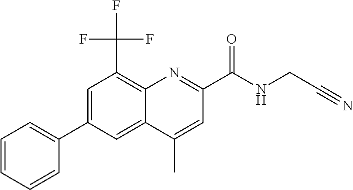

¹H NMR (400 MHz, dmso) δ 8.86 (t, J=5.8 Hz, 1H), 8.62 (s, 1H), 8.48 (s, 1H), 8.18 (s, 1H), 7.95 (d, J=7.3 Hz, 2H), 7.55 (t, J=7.5 Hz, 2H), 7.47 (t, J=7.3 Hz, 1H), 4.43 (d, J=5.8 Hz, 2H), 2.90 (s, 3H); ¹⁹F NMR (376 MHz, dmso) δ −58.16 (s); MS [M+H]⁺=370.05.

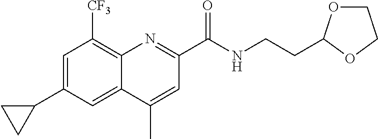

¹H-NMR (400 MHz, CH₃OH-d₄) δ 8.09 (s, 1H), 8.05 (s, 1H), 7.86 (s, 1H), 5.02 (m, 1H), 4.05 (m, 2H), 3.91 (m, 2H), 3.65 (m, 2H), 2.80 (s, 3H), 2.25 (m, 1H), 2.00 (m, 2H), 1.18 (m, 2H), 0.95 (m, 2H); ¹⁹F NMR (400 MHz, CH₃OH-d₄) δ −61.78 (s); MS [M+H]⁺=395.

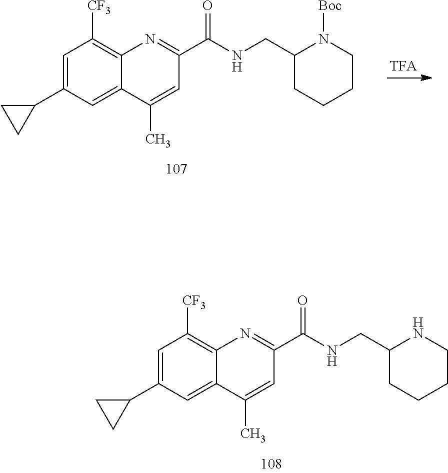

The compounds were made according to procedures described previously.

Compound 107: ¹H-NMR (400 MHz, CD₃OD) δ 8.44 (bs, 1H), 8.06 (s, 1H), 7.99 (s, 1H), 7.82 (s, 1H), 4.82 (m, 1H), 4.48 (m, 1H), 3.97 (m, 2H), 3.47 (m, 1H), 3.04 (m, 1H), 2.77 (s, 3H), 2.22 (m, 1H), 1.67 (m, 4H), 1.21 (m, 2H), 1.12 (s, 9H), 0.90 (m, 2H). ¹⁹F NMR (376.1 MHz) δ −61.34 (s); MS [M+H]⁺=491.84.

Compound 108: ¹H-NMR (400 MHz, CD₃OD) δ 8.68 (bs, 1H), 8.14 (s, 1H), 7.07 (s, 1H), 7.88 (s, 1H), 3.80-3.64 (m, 2H), 3.36 (m, 2H), 2.94 (m, 1H), 2.03-1.54 (m, 6H), 1.17 (m, 2H), 0.91 (m, 2H). ¹⁹F NMR (376.1 MHz) δ −61.54 (s), −77.51 (s, TFA); MS [M+H]⁺=392.07.

### Preparation of Compound 110

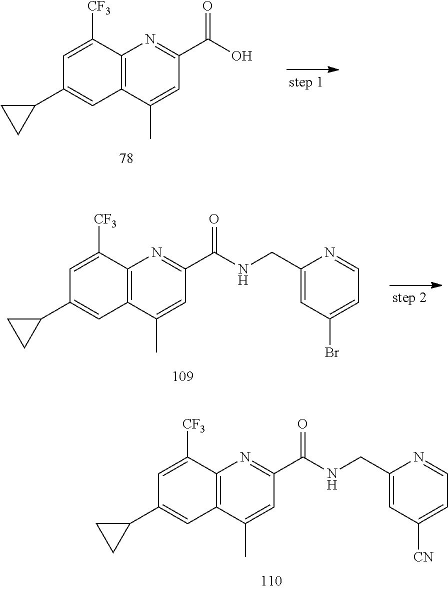

### Step 1

Compound 78 (0.200 g, 0.678 mmol) was dissolved in 5 ml of DMF in a 25 ml round bottom flask. HATU (0.516 mg, 1.36 mmol), N-methylmorpholine (0.373 ml, 3.39 mmol) and (4-bromopyridin-2-yl)methanamine (0.380 mg, 2.033 mmol) were added and the mixture was stirred at room temperature for 1 hour. The mixture was diluted with EtOAc. The organic solution was washed successively with concentrated NH₄Cl, water and brine and then dried over Na₂SO₄. The solution was concentrated under vacuum and the resulting solid was used in the next step without purification. MS [M+H]⁺=464.04

### Step 2

Compound 109 (0.678 mmol), zinc (II) cyanide (50 mg, 0.406 mmol) and Pd(PPh₃)₄ (40 mg, 0.034 mmol) were combined in a 25 ml round bottom flask. The reaction vessel was placed under vacuum and then refilled with Ar three times. DMF (4 ml) was added to the solid mixture. The reaction vessel was heated to 80° C. with stirring. The reaction was monitored by LC-MS, which showed complete conversion of the starting material after 2 hours. After the flask was cooled to room temperature, the mixture was purified by HPLC to give compound 110. ¹H-NMR (400 MHz, cdcl₃) δ 9.07 (s, 1H), 8.79 (s, 1H), 8.19 (s, 1H), 7.90 (s, 1H), 7.78 (s, 1H), 7.60 (s, 1H), 7.44 (d, J=4.3 Hz, 1H), 7.15 (d, J=7.4 Hz, 1H), 4.90 (d, J=5.8 Hz, 1H), 2.78 (s, 3H), 2.34 (s, 1H), 1.17 (dd, J=8.3, 1.5 Hz, 2H), 0.89 (dd, J=5.0, 1.4 Hz, 2H). MS [M+H]⁺=411.22.

### Preparation of Compound 112

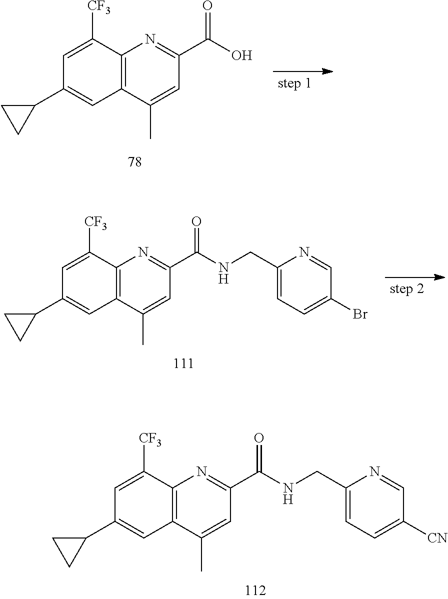

Compound 112. ¹H-NMR (400 MHz, CHCl₃-d) δ 9.10 (s, 1H), 8.88 (s, 1H), 8.17 (s, 1H), 7.99-7.84 (m, 2H), 7.78 (s, 1H), 7.49 (d, J=8.2 Hz, 1H), 7.36-7.20 (m, 1H), 7.15 (d, J=7.6 Hz, 1H), 4.91 (d, J=5.8 Hz, 2H), 2.77 (s, 6H), 2.33 (s, 1H), 2.21-2.12 (m, 1H), 1.17 (q, J=6.3 Hz, 2H), 0.88 (q, J=5.2 Hz, 2H). MS [M+H]⁺=411.20.

### Preparation of Compound 113

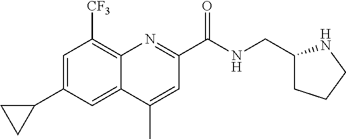

¹H-NMR (400 MHz, CH₃OH-d₄) δ 8.08 (s, 1H), 8.04 (s, 1H), 7.85 (s, 1H), 3.82 (m, 3H), 3.40 (m, 2H), 2.81 (s, 3H), 2.25 (m, 2H), 2.10 (m, 2H), 1.88 (m, 1H), 1.18 (m, 2H), 0.92 (m, 2H); ¹⁹F NMR (400 MHz, CH₃OH-d₄) δ −61.31 (s); MS [M+H]⁺=378.

### Preparation of Compound 116 to 118

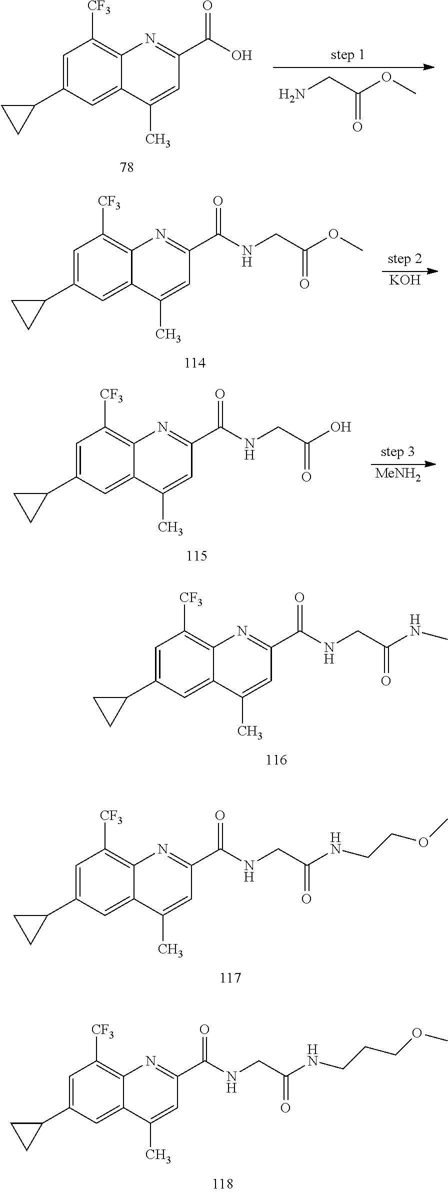

### Step 1

The compounds 114 was made using 6-cyclopropyl-4-methyl-8-trifluoromethyl-quinoline-2-carboxylic acid and amino-acetic acid methyl ester with HATU coupling according to procedure in example 79

### Step 2

Compound 114 (123 mg, 0.336 mmol) dissolved in THF (2 ml) and MeOH (0.1 mL) was added 1M KOH (0.672 mL). The reaction was stirred at RT for 1 h for completion. Reaction mixture was acidified with 1N HCl to pH ˜5. It was extracted with EtOAc, washed with brine. The organic layer was dried (MgSO₄) and concentrated to give 128 mg, 100% yield of compound 115.

### Step 3

Compound 115 (32 mg, 0.091 mmol) dissolved in DMF (1 ml) was added NMM (0.04 mL, 0.364 mmol), HATU (52 mg, 0.136 mmol) and methylamine (2M in THF) (0.09 mL, 0.182 mmol). The reaction was stirred at RT for 1 h for completion. Reaction mixture was diluted with EtOAc, washed with 3% LiCl (aq), sat'd NaHCO₃ and brine. The organic layer was dried (Na₂SO₄) and concentrated. The crude product was purified by flash chromatography on silica gel with EA/Hex to give 16 mg of compound 116.

Compound 116 ¹H-NMR (400 MHz, CHCl₃-d) δ 8.65 (bs, 1H), 8.15 (s, 1H), 7.88 (s, 1H), 7.77 (s, 1H), 6.22 (bs, 1H), 4.17 (d, 2H), 2.84 (d, 3H), 2.77 (s, 3H), 2.16 (m, 1H), 1.15 (m, 2H), 0.88 (m, 2H); ¹⁹F NMR (376.1 MHz) δ −60.34 (s); MS [M+H]⁺=366.08.

Compound 117 and 118 were made with same procedure using corresponding amines.

Compound 117 ¹H-NMR (400 MHz, CHCl₃-d) δ 8.67 (bs, 1H), 8.15 (s, 1H), 7.88 (s, 1H), 7.77 (s, 1H), 6.41 (bs, 1H), 4.18 (d, 2H), 3.47 (m, 4H), 3.30 (s, 3H), 2.77 (s, 3H), 2.16 (m, 1H), 1.15 (m, 2H), 0.88 (m, 2H); ¹⁹F NMR (376.1 MHz) δ −60.34 (s); MS [M+H]⁺=410.10.

Compound 118 ¹H-NMR (400 MHz, CHCl₃-d) δ 8.64 (bs, 1H), 8.17 (s, 1H), 7.88 (s, 1H), 7.77 (s, 1H), 6.70 (bs, 1H), 4.18 (d, 2H), 3.41 (m, 4H), 3.10 (s, 3H), 2.18 (s, 3H), 2.16 (m, 1H), 1.75 (m, 2H), 1.15 (m, 2H), 0.88 (m, 2H); ¹⁹F NMR (376.1 MHz) δ −60.33 (s); MS [M+H]⁺=423.13.

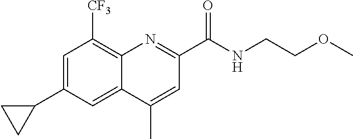

¹H-NMR (400 MHz, CH₃OH-d₄) δ 8.08 (s, 1H), 8.04 (s, 1H), 7.85 (s, 1H), 3.63 (m, 4H), 3.41 (s, 3H), 2.80 (s, 3H), 2.25 (m, 1H), 1.18 (m, 2H), 0.92 (m, 2H); ¹⁹F NMR (400 MHz, CH₃OH-d₄) δ −61.81 (s); MS [M+H]⁺=353.

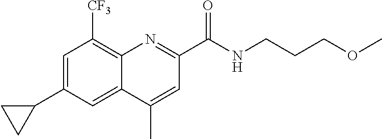

¹H-NMR (400 MHz, CH₃OH-d₄) δ 8.08 (s, 1H), 8.04 (s, 1H), 7.86 (s, 1H), 3.59 (m, 4H), 3.37 (s, 3H), 2.79 (s, 3H), 2.25 (m, 1H), 1.90 (m, 2H), 1.18 (m, 2H), 0.92 (m, 2H); ¹⁹F NMR (400 MHz, CH₃OH-d₄) δ −61.64 (s); MS [M+H]⁺=367.

### Preparation of Compounds 121-125

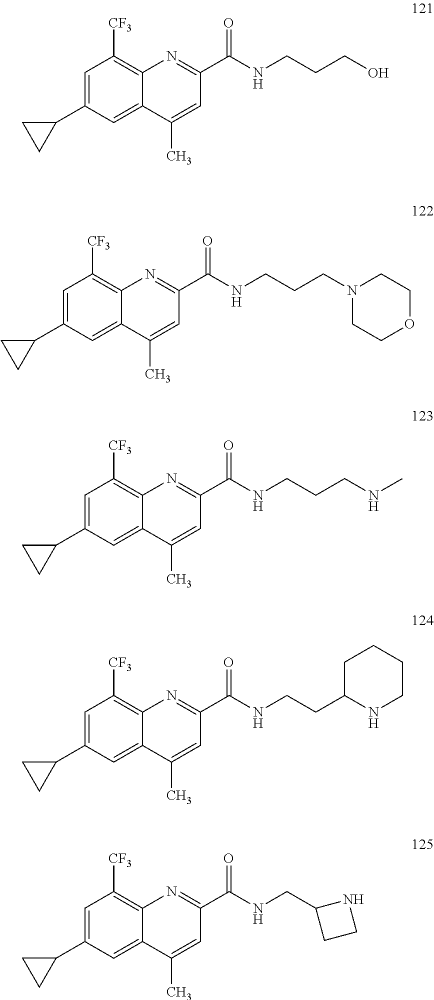

The compounds 35-40 were made using 6-cyclopropyl-4-methyl-8-trifluoromethyl-quinoline-2-carboxylic acid and corresponding amine by HATU coupling according to described procedure.

Compound 121 ¹H-NMR (400 MHz, CHCl₃-d) δ 8.55 (bs, 1H), 8.14 (s, 1H), 7.88 (s, 1H), 7.75 (s, 1H), 3.74 (m, 4H), 2.75 (s, 3H), 2.16 (m, 1H), 1.85 (m, 2H), 1.15 (m, 2H), 0.89 (m, 2H); ¹⁹F NMR (376.1 MHz) δ −60.33 (s); MS [M+H]⁺=353.14.

Compound 122 ¹H-NMR (400 MHz, CHCl₃-d) δ 8.30 (bs, 1H), 8.14 (s, 1H), 7.87 (s, 1H), 7.74 (s, 1H), 3.70 (m, 4H), 3.58 (m, 2H), 2.75 (s, 3H), 2.46 (m, 6H), 2.13 (m, 1H), 1.85 (m, 2H), 1.15 (m, 2H), 0.89 (m, 2H); ¹⁹F NMR (376.1 MHz) δ −60.31 (s); MS [M+H]⁺=422.20.

Compound 123 ¹H-NMR (400 MHz, CHCl₃-d) δ 9.85 (bs, 1H), 8.45 (bs, 1H), 8.09 (s, 1H), 7.88 (s, 1H), 7.75 (s, 1H), 3.64 (m, 2H), 3.05 (m, 2H), 2.75 (s, 3H), 2.73 (s, 3H), 2.16 (m, 3H), 1.85 (m, 2H), 1.15 (m, 2H), 0.89 (m, 2H); ¹⁹F NMR (376.1 MHz) δ −60.33 (s), 76.23 (s, TFA); MS [M+H]⁺=465.91.

Compound 124 ¹H-NMR (400 MHz, CHCl₃-d) δ 8.53 (bs, 1H), 7.86 (s, 1H), 7.76 (s, 1H), 7.74 (s, 1H), 4.85 (m, 1H), 4.43 (m, 1H), 3.25 (m, 1H), 2.91 (m, 2H), 2.72 (s, 3H), 2.41-1.69 (m, 8H), 1.15 (m, 2H), 0.89 (m, 2H); ¹⁹F NMR (376.1 MHz) δ −60.41 (s), 76.20 (s, TFA); MS [M+H]⁺=406.26.

Compound 125 ¹H-NMR (400 MHz, CHCl₃-d) δ 7.98 (m, 1H), 7.75 (m, 1H), 7.65 (m, 1H), 4.89 (m, 1H), 4.76 (m, 2H), 2.66 (m, 1H), 2.62 (m, 3H), 2.46 (m, 1H), 2.08 (m, 2H), 1.18 (m, 1H), 1.08 (m, 2H), 0.79 (m, 2H); ¹⁹F NMR (376.1 MHz) δ −61.10 (s); MS [M+H]⁺=364.09.

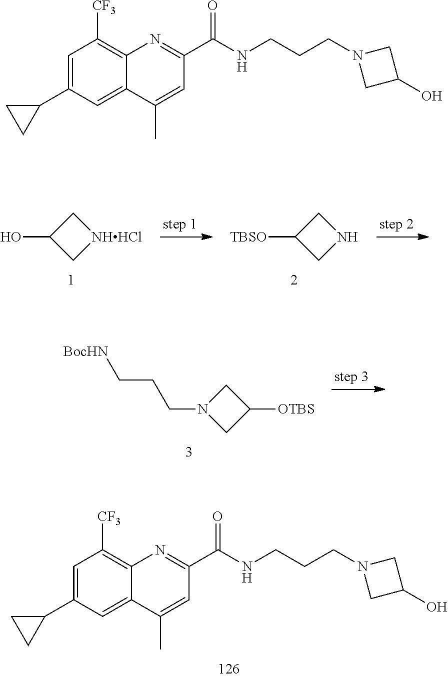

### Step 1

A 100-mL 1-neck rbf was charged with intermediate 1 (0.51 g, 9.1 mmol), imidazole (1.85 g, 27.3 mmol), and dichloromethane (20 mL). The reaction mixture was cooled to 0° C. with stirring and tert-butyldimethylsilyl (2.0 g, 13.7 mmol) was added in portion wise. The reaction mixture was stirred at room temperature for overnight. The reaction mixture was diluted with EtOAc (100 mL) and washed with sat.NaHCO₃, brine (2×50 mL) and dried with sodium sulfate. After removal of the solvent in vacuo, the crude residue was purified by silica gel chromatography with EtOAc/Hexane to give the desired compound 2 (1.5 g, 88%) as white solid MS [M+H]⁺=188.

### Step 2

A 50-mL 1-neck rbf was charged with intermediate 2 (0.51 g, 2.13 mmol), cesium carbonate (1.38 g, 4.26 mmol), and DMF (5 mL). (3-Bromo-propyl)-carbamic acid tert-butyl ester (0.4 g, 2.13 mmol) was added to the reaction mixture. The reaction mixture was stirred at 50° C. for 10 minutes and cooled to room temperature. The reaction crude was diluted with EtOAc (100 mL) and washed with sat.NaHCO₃, brine (2×50 mL) and dried with sodium sulfate. After removal of the solvent in vacuo, the crude residue was purified by silica gel chromatography with EtOAc/Hexane to give the desired compound 3 (0.7 g, 95%) as liquid. MS [M+H]⁺=345.

### Step 3

A 50-mL 1-neck rbf was charged with intermediate 3 (0.10 g, 0.29 mmol) and 4 N HCl in dioxane (3 mL). The reaction mixture was stirred at room temperature for 1 hour and concentrated to remove the solvent. The crude, HATU (0.12 g, 0.32 mmol), NMM (0.050 g, 0.48 mmol) and the acid part (0.05 g, 0.16 mmol) were dissolved in DMF (2 mL) in the 50-mL 1-neck rbf. The reaction mixture was stirred at room temperature for overnight and purified by HPLC to afford compound 126 (60 mg, 92%) as a white solid.

¹H-NMR (400 MHz, CH₃OH-d₄) δ 8.08 (s, 1H), 8.05 (s, 1H), 7.86 (s, 1H), 4.39 (m, 1H), 3.82 (m, 2H), 3.63 (m, 2H), 3.09 (m, 2H), 2.80 (s, 3H), 2.75 (m, 2H), 2.25 (m, 1H), 1.77 (m, 2H), 1.18 (m, 2H), 0.93 (m, 2H); ¹⁹F NMR (400 MHz, CH₃OH-d₄) δ −61.62 (s); MS [M+H]⁺=408.

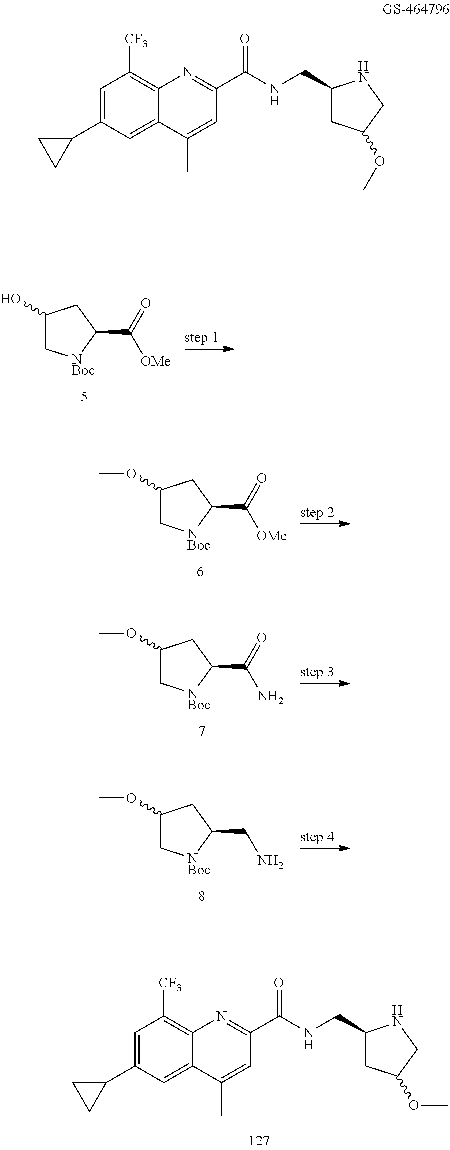

### Step 1

A 100-mL 1-neck rbf was charged with intermediate 5 (5.0 g, 20.4 mmol) and DMF (50 mL). NaH (0.98 g, 24.5 mmol, 60% in mineral oil) was added to the reaction mixture and followed by methyl iodide (4.3 g, 30.6 mmol). The reaction mixture was stirred at room temperature for 1 hour. The reaction mixture was diluted with EtOAc (100 mL) and washed with sat.NaHCO₃, brine (2×50 mL) and dried with sodium sulfate. After removal of the solvent in vacuo, the crude residue was purified by silica gel chromatography with EtOAc/Hexane to give the desired compound 6 (5.0 g, 94%) as liquid. MS [M+H]⁺=260.

### Step 2

A 250-mL 1-neck rbf was charged with intermediate 6 (5.0 g, 19.3 mmol), 1 M KOH (40 mL), and THF (40 mL). The reaction mixture was stirred at room temperature for 5 hours. The reaction mixture was acidified to pH=4 and extracted with EtOAc (200 mL) and dried with sodium sulfate. After removal of the solvent in vacuo, the crude residue was under high vacuum overnight. The crude, 4-methylmorphiline (2.2 g, 21.2 mmol), and THF (50 mL) were dissolved in a 250-mL 1-neck rbf. The reaction mixture was cooled to 0° C. and isobutylchloroformate (2.9 g, 21.2 mmol) was added slowly. The reaction mixture was stirred at 0° C. for 1 hour and diluted with EtOAc (100 mL) and washed with sat.NaHCO₃, brine (2×50 mL) and dried with sodium sulfate. After removal of the solvent in vacuo, the crude residue was purified by silica gel chromatography with EtOAc/Hexane to give the desired compound 7 (2.60 g, 52%) as liquid. MS [M+H]⁺=245.

### Step 3

A 250-mL 1-neck rbf was charged with intermediate 7 (1.8 g, 7.3 mmol) and THF (10 mL). The reaction mixture was cooled to 0° C. and 1 M borane in THF (30 mL) was added portion wise. The reaction mixture was heated to reflux with stirring for 2 hours and cooled to room temperature. MeOH (5 mL) was added drop wise to the reaction mixture. After removal of the solvent in vacuo, the crude was dissolved in EtOAc (100 mL) and washed with sat. NaHCO₃. After removal of the solvent, the crude compound 8 was under high vacuum overnight and used for next step without further purification. MS [M+H]⁺=231.

### Step 4

A 50-mL 1-neck rbf was charged with intermediate 8 (0.12 g, 0.51 mmol), HATU (0.12 g, 0.32 mmol), NMM (0.050 g, 0.48 mmol), the acid part (0.05 g, 0.16 mmol) and DMF (2 mL). The reaction mixture was stirred at room temperature for 1 hour and purified by HPLC. The intermediate was stirred in TFA (2 mL) for 1 hour and purified by HPLC to afford compound 127 (50 mg, 75%) as a white solid. ¹H-NMR (400 MHz, CH₃OH-d₄) δ 8.17 (m, 2H), 7.87 (s, 1H), 4.45-3.19 (m, 8H), 2.33 (m, 2H), 1.98-1.78 (m, 2H), 1.20 (m, 2H), 0.95 (m, 2H); ¹⁹F NMR (400 MHz, CH₃OH-d₄) δ −61.44 (s); MS [M+H]⁺=408.

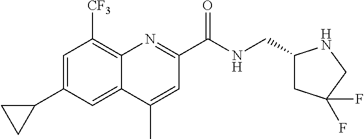

¹H-NMR (400 MHz, CH₃OH-d₄) δ 8.07 (s, 1H), 8.02 (s, 1H), 7.84 (s, 1H), 3.61 (m, 3H), 3.28 (m, 2H), 2.78 (s, 3H), 2.42 (m, 1H), 2.23 (m, 1H), 2.06 (m, 1H), 1.26 (m, 2H), 0.90 (m, 2H); ¹⁹F NMR (400 MHz, CH₃OH-d₄) δ −61.62 (s, 3 F), −98.54 (m, 2 F); MS [M+H]⁺=414.

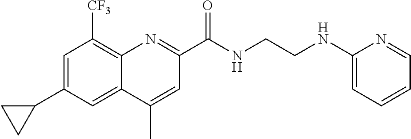

¹H-NMR (400 MHz, CDCl3) δ 8.17 (t, 1H), 8.16 (s, 1H), 7.95 (d, 1H), 7.88 (d, 1H), 7.75 (s, 1H), 7.51 (t, 1H), 6.70 (d, 1H), 6.59 (t, 1H), 3.76 (m, 2H), 3.64 (m, 2H), 2.16 (m, 1H), 1.33 (m, 2H), 1.15 (m, 2H), 0.88 (m, 3H); ¹⁹F NMR (376.1 MHz) δ −60.38 (s), −76.57 (s); MS [M−H]⁺=415.20.

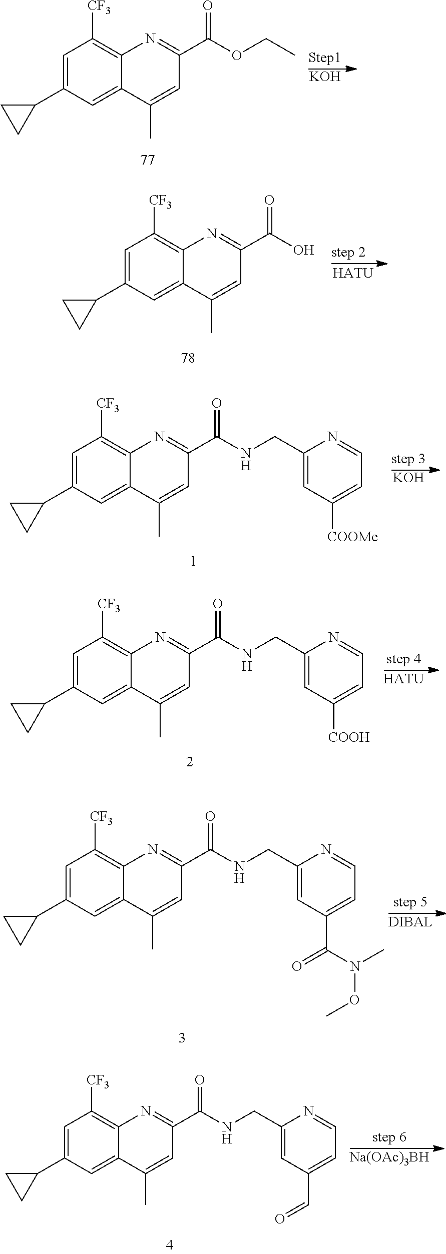

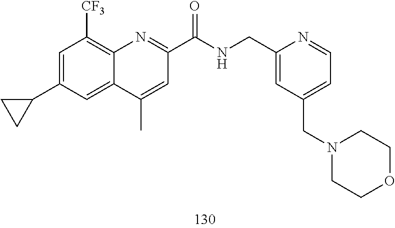

### Step 1, 2, 3 and 4

The procedures were described previously.

### Step 5

Compound 6-Cyclopropyl-4-methyl-8-trifluoromethyl-quinoline-2-carboxylic acid [4-(methoxy-methyl-carbamoyl)-pyridin-2-ylmethyl]-amide 3 (0.136 g, 0.28 mmol) was dissolved in THF (2 ml) and the solution was cooled to −78° C. and followed by the addition of DIBAL (1N in THF) (0.43 mmol). The resulting reaction mixture was stirred at −78° C. for 4 hours. More DIBAL (0.14 mmol) was added to the reaction mixture. One hour later, cooling bath was removed and NH₄Cl (sat.) (4 ml) was added to the reaction and followed by EtOAc (4 ml) and Rochelle salts (sat.) (4 ml) and the resulting mixture was stirred at room temperature for 40 minutes to separate the two phases. Organic phase was dried with sodium sulfate. After removal of the solvent in vacuo, compound 4 was obtained and no further purification was performed.

### Step 6

Compounds 6-Cyclopropyl-4-methyl-8-trifluoromethyl-quinoline-2-carboxylic acid (4-formyl-pyridin-2-ylmethyl)-amide 4 (0.03 g, 0.073 mmol) and Morpholine (0.0095 g, 0.11 mmol) were dissolved in DCM (1 ml) and the resulting mixture was stirred at rt for 1 hour. Na(OAc)₃BH (0.038 g, 0.182 mmol) was added to the reaction mixture and followed by the addition of catalytic amount of AcOH. One hour later, the reaction mixture was diluted with DCM (2 ml) and washed with NaHCO₃ (sat.), H₂O and Brine. Solvent was removed under vacuo and the residue was purified by HPLC to obtain compound 130 (0.032 g, 90%).

Compound 130: ¹H-NMR (400 MHz, CDCl3) δ 9.04 (t, 1H), 8.76 (br, 1H), 8.04 (s, 1H), 7.86 (s, 1H), 7.81 (s, 1H), 7.76 (m, 2H), 7.69 (d, 1H), 4.99 (d, 2H), 4.30 (s, 2H), 3.92 (s, 4H), 3.19 (s, 4H), 2.72 (s, 3H), 2.16 (m, 1H), 1.15 (m, 2H), 0.88 (m, 2H); ¹⁹F NMR (376.1 MHz) δ −60.49 (s), −76.53 (s); MS [M−H]⁺=485.40.

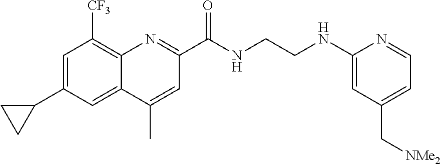

Compound 131: ¹H-NMR (400 MHz, CDCl3) δ 9.04 (t, 1H), 8.76 (br, 1H), 8.10 (s, 1H), 7.88 (s, 1H), 7.76 (s, 1H), 7.59 (m, 2H), 7.55 (d, 1H), 4.93 (d, 2H), 4.24 (s, 2H), 2.81 (s, 9H), 2.76 (s, 3H), 2.16 (m, 1H), 1.15 (m, 2H), 0.88 (m, 2H); ¹⁹F NMR (376.1 MHz) δ −60.41 (s), −76.43 (s); MS [M−H]⁺=404.17.

### Preparation of Compound 132

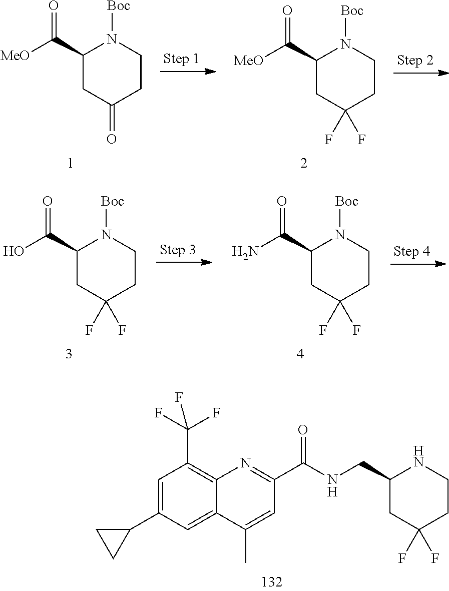

### Step 1

A solution of compound 1 (2.17 g, 8.43 mmol) in dichloromethane (100 mL) was stirred at −78° C. bath as deoxofluoro (3.6 mL, 19.53 mmol) was added dropwise. The solution was stirred for 3 h at the cold bath and at it for 16 h. The solution was cooled to 0° C. and some ice was added to the solution. After 10 min, saturated aq. NaHCO₃ solution was added. After two fractions were separated, the aqueous fraction was extracted with dichloromethane (30 mL×2) and combined organic fractions were dried (MgSO₄) and concentrated. The residue was purified by combiflash using hexanes and ethyl acetate to obtain compound 2 (1.554 g, 66%) with some impurities.

### Step 2

A solution of compound 2 (1.526 g, 5.462 mmol) in THF (11 mL), methanol (11 mL), and 1 N KOH (11 mL, 11 mmol) was stirred at it for 1.5 h and then additional 1 N KOH (5.5 mL, 5.5 mmol) was added. After 1 h, the mixture was concentrated to a half volume and diluted with water before washing with ether (×1). The aqueous solution was acidified with 1 N HCl (20 mL) and the product was extracted with ethyl acetate (×2). The extracts were washed with brine (×1), combined, dried (Na₂SO₄), and concentrated to obtain compound 3 (1.429 g, 98%). MS [M−H]⁻=264.2

### Step 3

A solution of compound 3 (1.429 g, 5.39 mmol) and N-methylmorpholine (1.8 mL, 16.37 mmol) in THF (25 mL) was stirred at ice-salt bath as isobutyl chloroformate (0.79 mL, 6.04 mmol) was added dropwise. After 30 min, concentrated NH₄OH (3 mL) was added and the mixture was stirred in the cold bath for 1 h and at rt for 1 h. The solution was diluted with water, acidified with concentrated HCl, then product was extracted with ethyl acetate (×2). The extracts were washed with brine (×1), combined, dried (Na₂SO₄), and concentrated. The residue was purified by combiflash using hexanes and ethyl acetate to obtain compound 4 (1.273 g, 89%) with some impurities.

### Step 4

A solution of compound 4 (325 mg, 1.23 mmol) in dichloromethane (3 mL) and 4 N HCl in dioxane (3 mL, 12 mmol) was stirred at it for 1 h and concentrated. After the residue was dried in vacuum for 30 min, the residue was stirred with THF (5 mL) at it as 1 M LiAlH₄ solution in ether (5 mL, 5 mmol) was added. The resulting solution was refluxed for 4 h and then stirred at 0° C. After the mixture was diluted with THF (6 mL), it was quenched by adding slowly water (0.19 mL, 15% NaOH (0.19 mL), and water (0.57 mL) sequentially. The resulting mixture was stirred for 30 min at 0° C. and filtered through celite pad. After the filtrate was concentrated, the residue was dissolved in ethyl acetate, dried (Na₂SO₄), and concentrated again.

The residue, compound 78 (53 mg, 0.181 mmol), and HATU (108 mg, 0.284 mmol) were dissolved in DMF (3 mL) and stirred at 0° C. as N-methylmorpholine (0.06 mL, 0.546 mmol) was added. After the mixture was stirred for 30 min at 0° C. and for 1 h at rt, it was diluted with 5% aqueous LiCl solution, and extracted with ethyl acetate (×2). The organic fractions were washed with water (×1), combined, dried (Na₂SO₄), and concentrated. The residue was partially purified by combiflash using hexanes and ethyl acetate. The partially purified product was further purified by preparative HPLC followed by repeated preparative TLC to obtain compound 132 (20 mg, 26%). ¹H-NMR (400 MHz, CDCl₃) δ 8.54 (br t, 1H), 8.16 (s, 1H), 7.88 (s, 1H), 7.77 (s, 1H), 3.56 (t, J=5.6 Hz, 2H), 3.08-3.21 (m, 2H), 2.86 (br t, J=11.4 Hz, 1H), 2.77 (s, 3H), 2.10-2.20 (m, 2H), 1.99-2.10 (m, 1H), 1.57-1.92 (m, 4H), 1.14-1.20 (m, 2H), 0.86-0.92 (m, 2H); ¹⁹F NMR (376.1 MHz, CDCl₃) δ −60.40 (s, 3 F), −88.62 (d, J=236.2 Hz, 1 F), −101.62 (dtt, J=236.9, 33.8, and 11.7 Hz, 1 F); MS [M+H]⁺=428.1

### Preparation of Compounds 133-134

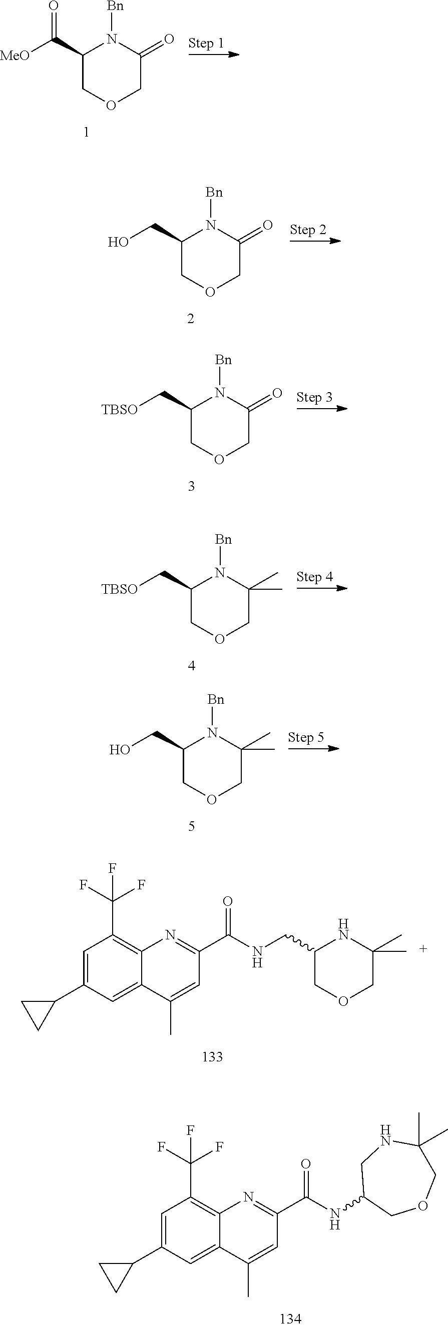

### Step 1

A suspension of compound 1 (2.001 g, 8.027 mmol) and NaBH₄ (762 mg, 20.14 mmol) in THF (32 mL) was stirred at 55° C. bath as methanol (6.5 mL) was added over 30 min. After 30 min, water (20 mL) was added to the mixture and the solution was concentrated to remove organic solvents. After the resulting mixture was diluted with brine (30 mL), the product was extracted with ether (×2) and the extracts were washed with brine (×2), combined, dried (Na₂SO₄), and concentrated. The crude residue 2 was used for the next reaction. MS [M+H]⁺=222.1

### Step 2

A solution of the crude 2, TBSCl (1.468 g, 9.739 mmol), and imidazole (839 mg, 12.32 mmol) in dichloromethane (20 mL) was stirred at rt for 1 h. The mixture was diluted with water and the product was extracted with ethyl acetate (×2), washed with water (×1), dried (Na₂SO₄), and concentrated to obtain 1.003 g (37% for 2 steps) of compound 3. MS [M+H]⁺=336.3

### Step 3

To a solution of compound 3 (1.003 g, 2.99 mmol) in THF (6 mL) was added ZrCl₄ (700 mg, 3.00 mmol) at −10° C. The mixture was stirred at −10° C. for 30 min and 3 M solution of MeMgBr in ether was added dropwise. The resulting thick mixture was stirred at rt for 4 h. The mixture was mixed with ether and aqueous solution of Na, K tartarate and the mixture was filtered through celite pad. The two phases of the filtrate were separated and the aqueous fraction was extracted with ethyl acetate (×1). The organic fractions were washed with water, combined, dried (Na₂SO₄), and concentrated. The residue was purified by combiflash using hexanes and ethyl acetate to obtain compound 4 (811 mg, 78%). MS [M+H]⁺=350.3

### Step 4

A solution of compound 4 (807 mg, 2.309 mmol)in THF (5 mL) was stirred at it as 1 M tetrabutylammonium fluoride in THF (2.6 mL, 2.6 mmol) was added. After 1 h stirring, additional 1 M tetrabutylammonium fluoride in THF (2.6 mL, 2.6 mmol) was added. After stirring at it overnight, The solution was diluted with water and the product was extracted with ethyl acetate (×2). The organic fractions were washed with water (×1), combined, dried (Na₂SO₄), and concentrated. The residue was purified by combiflash using hexanes and ethyl acetate to obtain compound 5 (567 mg, quantitative). MS [M+H]⁺=236.1

### Step 5

A solution of compound 5 (152 mg, 0.646 mmol) and triethylamine (0.15 mL, 1.076 mmol) in dichloromethane (6 mL) was stirred at an ice-salt bath as methanesulfonyl chloride (0.07 mL, 0.900 mmol) was added dropwise. After 30 min, the mixture was diluted with ice-cold dichloromethane (˜10 mL) and washed with ice-cold saturated aq. NaHCO₃ solution. The aqueous fraction was extracted with ice-cold dichloromethane (×1). The organic fractions were washed with ice-cold brine (×1), combined, dried (MgSO₄), and concentrated to ˜½ volume. The concentrated solution was diluted with DMF (˜5 mL) before addition of sodium azide (210 mg, 3.23 mmol). The resulting mixture was cautiously concentrated to remove remained dichloromethane. The remained solution was stirred at 50° C. bath for 18 h. The mixture was diluted with 5% aq. LiCl solution and the product was extracted with ethyl acetate (×2). The organic fractions were washed with water (×1), combined, dried (Na₂SO₄), and concentrated. The crude residue contained two products with the same mass and the crude mixture was used for the next reaction. MS [M+H]⁺=261.1 The previous crude mixture and Pd(OH)₂/C (20 mg) in methanol (10 mL) and c. HCl (0.5 mL) was stirred vigorously under H₂ atmosphere for 3.5 h. The mixture was filtered through celite and the filtrate was concentrated. The resulting residue was concentrated with toluene twice and dried in vacuum.

The residue, compound 78 (148 mg, 0.500 mmol), and HATU (282 mg, 0.742 mmol) were dissolved in DMF (5 mL) and stirred at rt as N-methylmorpholine (0.55 mL, 5.00 mmol) was added. After 30 min at rt, the solution was diluted with 5% aqueous LiCl solution, and extracted with ethyl acetate (×2). The organic fractions were washed with water (×1), combined, dried (Na₂SO₄), and concentrated. The residue was purified by preparative HPLC to obtain compound 133 (83 mg, 25%) and 134 (129 mg, 40%).

Compound 133: ¹H-NMR (400 MHz, CDCl₃) δ ˜9.7 (br, 2H), 8.46 (br t, 1H), 8.02 (s, 1H), 7.81 (s, 1H), 7.74 (s, 1H), 4.14 (d, J=11.2 Hz, 1H), 3.82-3.92 (m, 2H), 3.66-3.82 (m, 2H), 3.70 (d, J=12.4 Hz, 1H), 3.59 (d, J=12.4 Hz, 1H), 2.69 (s, 3H), 2.15 (m, 1H), 1.54 (s, 3H), 1.42 (s, 3H), 1.15-1.21 (m, 2H), 0.85-0.91 (m, 2H); ¹⁹F NMR (376.1 MHz, CDCl₃) δ −60.31 (s, 3 F), −76.37 (s, 6 F); MS [M+H]⁺=422.2;

Compound 134: ¹H-NMR (400 MHz, CDCl₃) δ 9.73 (br, 2H), 8.34 (d, J=7.2 Hz, 1H), 7.80 (s, 1H), 7.71 (s, 1H), 7.61 (s, 1H), 4.83 (br, 1H), 4.26 (dd, J=12.0 and 6.0 Hz, 1H), 3.87 (d, J=13.2 Hz, 1H), 3.77-3.86 (m, 2H), 3.68 (d, J=13.2 Hz, 1H), 3.56 (m, 1H), 2.41 (s, 3H), 2.12 (m, 1H), 1.51 (s, 3H), 1.45 (s, 3H), 1.15-1.21 (m, 2H), 0.84-0.89 (m, 2H); ¹⁹F NMR (376.1 MHz, CDCl₃) δ −60.40 (s, 3 F), −76.18 (s, 6 F); MS [M+H]⁺=422.2

### Example 18

### Preparation of Compound 138

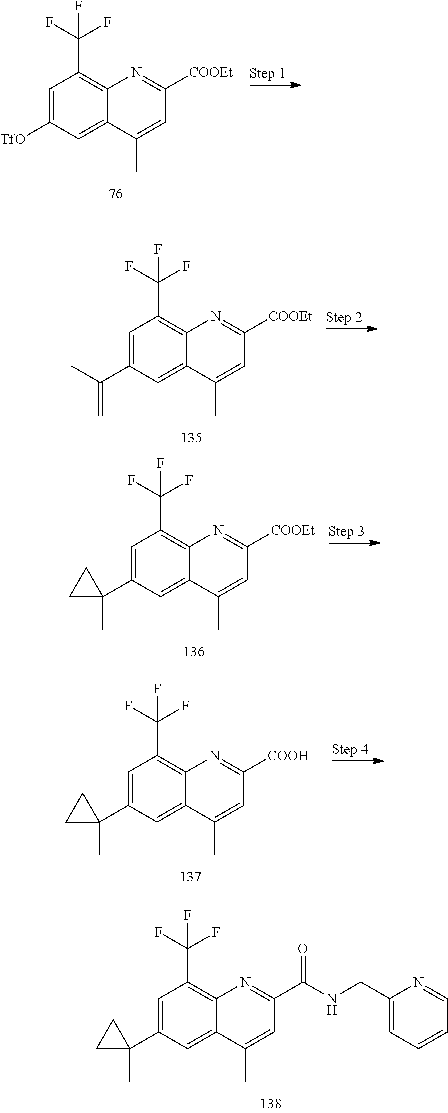

### Step 1

Compound 135 (261 mg, 83%) was prepared from 76 in a manner similar to that described in the synthesis of compound 79, except isopropenylboronic acid pinacol ester was used in place of cyclopropylboronic acid. MS [M+H]⁺=324.1

### Step 2

A mixture of compound 135 (101 mg, 0.313 mmol) and Pd(OAc)₂ (1.7 mg, 0.0076 mmol) in CH₂Cl₂ (1.5 mL) was stirred at 0° C. as a solution of CH₂N₂ in ether (˜4 mL) was added. After 1 h at 0° C., the mixture was warmed to rt and filtered through a celite pad. The filtrate was concentrated and the residue was purified by combiflash using hexanes and ethyl acetate to obtain compound 136 (84 mg, 79%). MS [M+H]⁺=338.1

### Step 3

Compound 137 (77 mg, quantitative) was prepared from 136 in a manner similar to that described in the synthesis of compound 78. MS [M+H]⁺=310.1

### Step 4

Compound 138 (26 mg, 97%) was prepared from 137 in a manner similar to that described previously. ¹H-NMR (400 MHz, CD₃OD) δ 9.15 (br t, J=5.2 Hz, 1H), 8.63 (d, J=4.8 Hz, 1H), 8.22 (s, 1H), 8.05 (d, J=1.6 Hz, 1H), 7.97 (s, J=1.2 Hz, 1H), 7.71 (td, J=8.0 and 2.0 Hz, 1H), 7.40 (d, J=7.6 Hz, 1H), 7.24 (dd, J=6.8 and 5.2 Hz, 1H), 4.89 (d, J=5.6 Hz, 2H), 2.80 (s, 3H), 1.54 (s, 3H), 1.02 (m, 2H), 0.92 (m, 2H); ¹⁹F NMR (376.1 MHz, CD₃OD) δ −60.32 (s, 3 F); MS [M+H]⁺=400.2

The following compounds were prepared in the manner similarly to 135

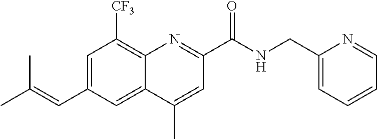

¹H-NMR (400 MHz, CH₃OH-d₄) δ 8.52 (m, 1H), 8.16 (s, 1H), 8.11 (s, 1H), 8.02 (s, 1H), 7.80 (m, 1H), 7.42 (m, 1H), 7.33 (m, 1H), 6.52 (s, 1H), 4.75 (s, 2H), 2.80 (s, 3H), 2.05 (m, 6H); ¹⁹F NMR (400 MHz, CH₃OH-d₄) δ −61.75 (s); MS [M+H]⁺=400.

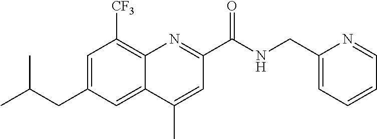

Compound 140 were obtained by hydrogenation of compound 139

¹H-NMR (400 MHz, CH₃OH-d₄) δ 8.71 (m, 1H), 8.43 (m, 1H), 8.11 (s, 1H), 8.07 (s, 1H), 8.03 (s, 1H), 7.93 (m, 1H), 7.80 (m, 1H), 4.99 (s, 2H), 2.81 (m, 5H), 2.05 (m, 1H), 0.99 (m, 6H); ¹⁹F NMR (400 MHz, CH₃OH-d₄) δ −61.46 (s); MS [M+H]⁺=402.

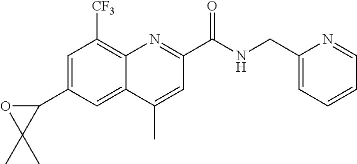

Compound 141 were obtained by epoxidation of compound 139

¹H-NMR (400 MHz, CH₃OH-d₄) δ 8.57 (m, 1H), 8.33 (s, 1H), 8.20 (s, 1H), 8.17 (s, 1H), 7.80 (m, 1H), 7.45 (m, 1H), 7.36 (m, 1H), 4.79 (s, 2H), 4.20 (s, 1H), 2.84 (s, 3H), 1.54 (s, 3H), 1.08 (s, 3H); ¹⁹F NMR (400 MHz, CH₃OH-d₄) δ −61.72 (s);

MS [M+H]⁺=416.

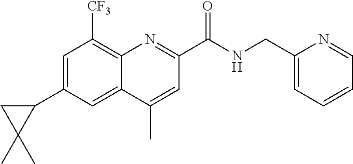

Compound 142 was prepared from compound 139 similarly to compound 136

¹H-NMR (400 MHz, CH₃OH-d₄) δ 8.69 (m, 1H), 8.33 (m, 1H), 8.11 (s, 1H), 8.07 (s, 1H), 8.03 (s, 1H), 7.89 (m, 1H), 7.76 (m, 1H), 4.97 (s, 2H), 2.81 (s, 3H), 2.21 (m, 1H), 1.32 (s, 3H), 1.15 (m, 2H), 0.99 (m, 2H), 0.83 (s, 3H); ¹⁹F NMR (400 MHz, CH₃OH-d₄) δ −61.92 (s); MS [M+H]⁺=414.

### Preparation of Compound 143

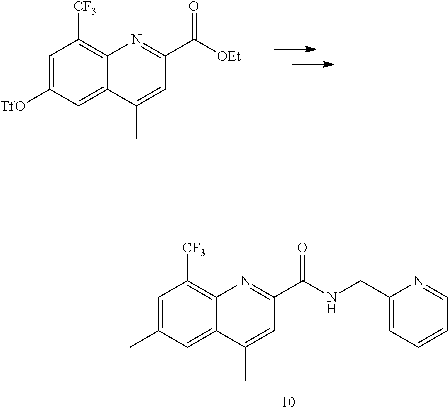

¹H-NMR (400 MHz, CDCl₃) δ 9.21 (m, 1H), 9.17 (br, 1H), 8.63 (m, 1H), 8.23 (s, 1H), 8.00 (s, 1H), 7.95 (s, 1H), 7.68 (m, 1H), 7.36 (m, 1H), 7.21 (m, 1H), 4.86 (d, 2H), 2.79 (s, 3H), 2.64 (s, 3H); ¹⁹F NMR (376.1 MHz) δ −60.37 (s); MS [M+H]⁺=360.13.

### Preparation of Compound 144

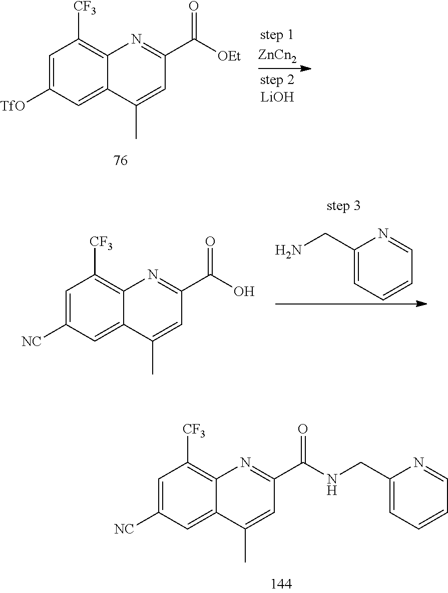

### Step 1

Intermediate 76 (254 mg, 0.589 mmol), Zinc cyanide (42.3 mg, 0.353 mmol), tetrakis(triphenylphosphine)palladium(0) (34.0 mg, 0.030 mmol) in DMF (3 mL) was degased with nitrogen three times. The reaction mixture was heated up to 80° C. under nitrogen with stirring for 60 mins. After cooling to RT, the reaction mixture was diluted with EtOAc (100 mL) and washed with 3% LiCl/water and brine, and dried with sodium sulfate. After removal of the solvent in vacuo, the residue was purified by preparative flash chromatography (silica gel, ethyl acetate/hexane gradient) affording 130 mg of ester as white solids, 72%.

Step 2 and step 3 was done as described, to afford compound 144. ¹H-NMR (400 MHz, DMSO-d6) δ 9.21 (m, 1H), 9.18 (s, 1H), 8.66 (s, 1H), 8.61 (m, 1H), 8.29 (s, 1H), 7.90 (t, 1H), 7.50 (d, 1H), 7.40 (m, 1H), 4.77 (d, 2H), 2.90 (s, 3H); ¹⁹F NMR (376.1 MHz) δ −59.31 (s); MS [M+H]⁺=371.15.

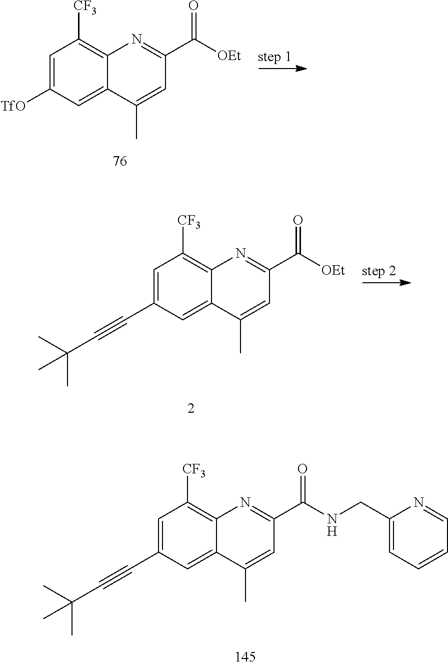

### Step 1

A 100-mL 1-neck rbf was charged with intermediate 76 (0.20 g, 0.46 mmol), 3,3-dimethyl-1-butyne (0.038 g, 0.46 mmol), tetrakis(triphenylphosphine)palladium(0) (0.010 g, 0.0092 mmol), catalytic amount copper iodide (5 mg) and triethylamine (3 mL). The reaction mixture was heated up to 45° C. with stirring for 2 hours. The reaction mixture was diluted with EtOAc (100 mL) and washed with water (2×50 mL) and dried with sodium sulfate. After removal of the solvent in vacuo, the crude residue was used directly for next step MS [M+H]⁺=364.

### Step 2

A 50-mL 1-neck rbf was charged with intermediate 2 (0.060 g, 0.16 mmol), 1 M potassium hydroxide (0.5 mL) and THF (1 mL). The reaction mixture was stirred at room temperature for 1 hour and acidified to pH=4 by adding 1M hydrogen chloride solution in water. The reaction crude was extracted with EtOAc (2×30 mL) and the combined organic layer was dried with sodium sulfate. After removal of the solvent in vacuo, the crude acid and 2-(aminomethyl)pyridine (0.034 g, 0.32 mmol), HATU (0.12 g, 0.32 mmol), NMM (0.050 g, 0.48 mmol) were dissolved in DMF (3 mL) in a 25-mL 1-neck rbf. The reaction mixture was stirred at room temperature for overnight and purified by HPLC to afford compound 145 (60 mg, 88%) as a white solid. ¹H-NMR (400 MHz, CH₃OH-d₄) δ 8.57 (m, 1H), 8.25 (s, 1H), 8.09 (s, 1H), 8.01 (s, 1H), 7.83 (m, 1H), 7.43 (m, 1H), 7.38 (m, 1H), 4.78 (m, 2H), 2.77 (s, 3H), 1.40 (s, 9H); ¹⁹F NMR (400 MHz, CH₃OH-d₄) δ −61.82 (s); MS [M+H]⁺=426.

Compound 146 to 149 were prepared in a manner similar to that described previously.

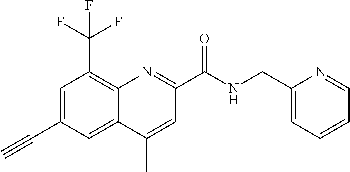

¹H-NMR (400 MHz, CH₃OH-d₄) δ 8.61 (m, 2H), 8.22 (m, 2H), 7.87 (m, 1H), 7.47 (m, 1H), 7.39 (m, 1H), 4.79 (s, 2H), 3.39 (s, 1H), 2.86 (s, 3H); ¹⁹F NMR (400 MHz, CH₃OH-d₄) δ −61.49 (s); MS [M+H]⁺=370.

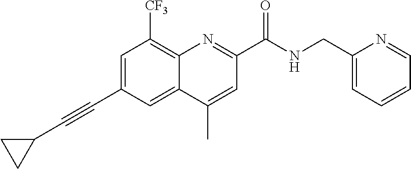

¹H-NMR (400 MHz, CH₃OH-d₄) δ 8.64 (m, 1H), 8.34 (s, 1H), 8.23 (m, 1H), 8.13 (s, 1H), 8.05 (s, 1H), 7.79 (m, 1H), 7.68 (m, 1H), 4.93 (s, 2H), 2.79 (s, 3H), 1.56 (m, 1H), 0.97 (m, 2H), 0.84 (m, 2H); ¹⁹F NMR (400 MHz, CH₃OH-d₄) δ −61.88 (s); MS [M+H]⁺=410.

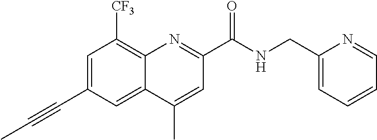

¹H-NMR (400 MHz, CH₃OH-d₄) δ 8.53 (m, 1H), 8.35 (s, 1H), 8.13 (s, 1H), 8.05 (s, 1H), 7.82 (m, 1H), 7.45 (m, 1H), 7.31 (m, 1H), 4.78 (s, 2H), 2.78 (s, 3H), 2.11 (s, 3H); ¹⁹F NMR (400 MHz, CH₃OH-d₄) δ −61.95 (s); MS [M+H]⁺=384.

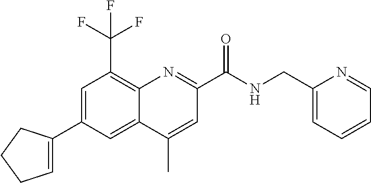

¹H-NMR (400 MHz, CH₃OH-d₄) δ 8.72 (m, 1H), 8.42 (m, 2H), 8.22 (m, 2H), 7.92 (m, 1H), 7.80 (m, 1H), 6.63 (s, 1H), 5.03 (s, 2H), 2.90 (m, 2H), 2.83 (s, 3H) 2.66 (m, 2H), 2.18 (m, 1H); ¹⁹F NMR (400 MHz, CH₃OH-d₄) δ −61.19 (s); MS [M+H]⁺ =412.

### Compound 150

Compound 150 was obtained from 149 by hydrogenation.

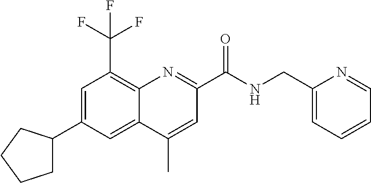

¹H-NMR (400 MHz, CH₃OH-d₄) δ 8.71 (m, 1H), 8.40 (m, 1H), 8.22 (s, 1H), 8.18 (m, 2H), 7.95 (m, 1H), 8.80 (m, 1H), 5.03 (s, 2H), 3.33 (m, 1H), 2.85 (s, 3H), 2.29-1.76 (m, 8H); ¹⁹F NMR (400 MHz, CH₃OH-d₄) δ −61.09 (s); MS [M+H]⁺=414.

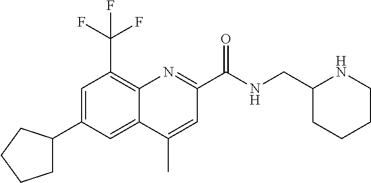

¹H-NMR (400 MHz, CH₃OH-d₄) δ 8.23 (s, 1H), 8.18 (s, 1H), 8.10 (s, 1H), 3.81-3.62 (m, 2H), 3.43 (m, 3H), 2.97 (m, 1H), 2.83 (s, 3H), 2.29-1.56 (m, 14H); ¹⁹F NMR (400 MHz, CH₃OH-d₄) δ −61.02 (s); MS [M+H]⁺=420.

### Preparation of Compound 152

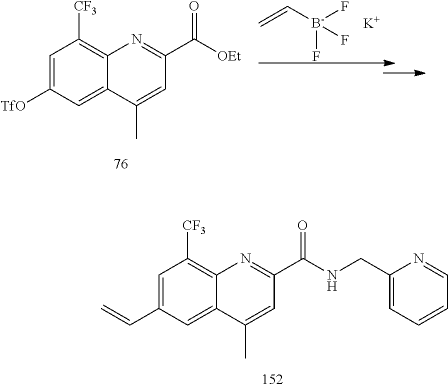

Compound 152 was made in the same manner.

¹H-NMR (400 MHz, CDCl₃) δ 9.18 (br, 1H), 8.64 (m, 1H), 8.25 (br, 1H), 8.09 (m, 1H), 7.68 (m, 1H), 7.36 (m, 1H), 7.21 (m, 1H), 6.94 (m, 1H), 6.00 (d, 1H), 5.53 (d, 1H), 4.87 (d, 2H), 2.81 (s, 3H); ¹⁹F NMR (376.1 MHz) δ −60.60 (s); MS [M+H]⁺=372.15.

### Preparation of Compound 154

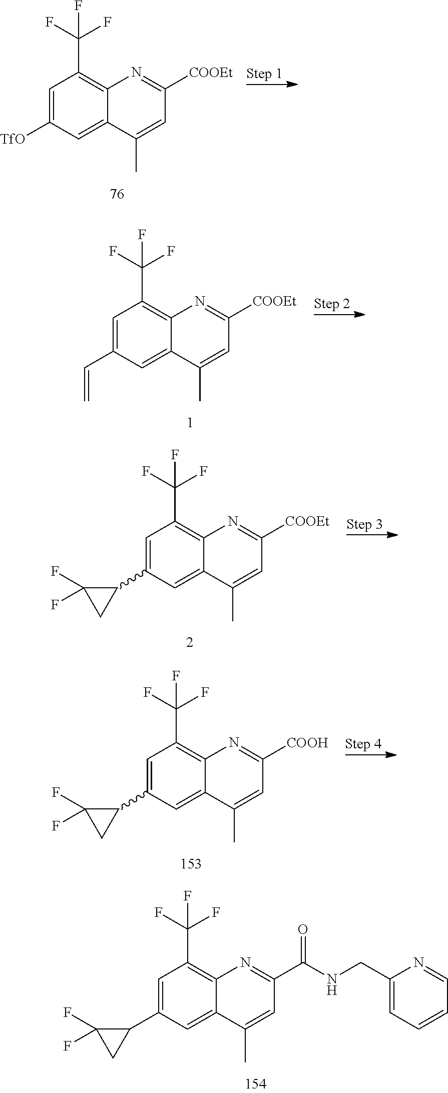

### Step 1

Compound 1 (365 mg, 76%) was prepared from 76. MS [M+H]⁺=310.1

### Step 2

A mixture of compound 1 (364 mg, 1.176 mmol) and sodium fluoride (1.3 mg, 0.031 mmol) in toluene (0.7 mL) was stirred at 110° C. as FSO₂CF₂COOTMS (0.6 mL, 3.045 mmol) was added over 5 h. The mixture was mixed with water containing some NaHCO₃ and extracted with dichloromethane (×2). The organic fractions were washed with water (×1), combined, dried (Na₂SO₄), and concentrated. The residue was purified by combiflash using hexanes and ethyl acetate to obtain compound 2 (327 mg, 77%). MS [M+H]⁺=360.1

### Step 3

Compound 153 (300 mg, quantitative) was prepared from 2. MS [M+H]⁺=332.1

### Step 4

Compound 154 (102 mg, 91%) was prepared from compound 71 (89 mg, 0.267 mmol) in a manner similar to that described in the synthesis of compound 14. ¹H-NMR (400 MHz, CDCl₃) δ 9.18 (br t, 1H), 8.62 (br d, J=3.6 Hz, 1H), 8.24 (s, 1H), 8.05 (s, 1H), 7.95 (s, 1H), 7.66 (td, J=7.6 and 1.2 Hz, 1H), 7.34 (d, J=7.6 Hz, 1H), 7.20 (br t, J=˜6 Hz, 1H), 4.85 (d, J=5.6 Hz, 2H), 2.99 (td, J=12.0 and 8.0 Hz, 1H), 2.78 (s, 3H), 1.96-2.06 (m, 1H), 1.76-1.85 (m, 1H); ¹⁹F NMR (376.1 MHz, CDCl₃) δ −60.46 (s, 3 F), −126.26 (dtd, J=155.5, 12.4 and 3.8 Hz, J=155.5, 12.6 and 5.5 Hz, 1 F), −141.96 (ddd, J=155.5, 12.6 and 5.5 Hz, 1 F); MS [M+H]⁺=422.2

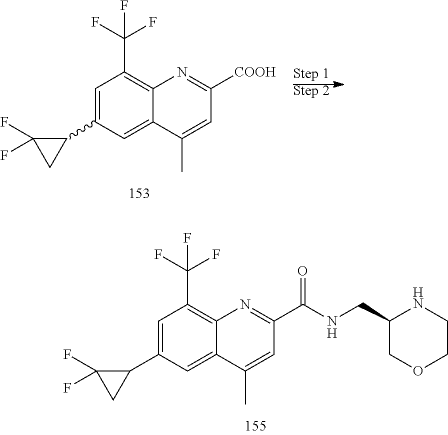

Compound 155 (139 mg, 96%) was prepared from compound 153 in two steps. ¹H-NMR (400 MHz, CDCl₃) δ 8.48 (br t, 1H), 8.20 (s, 1H), 8.05 (s, 1H), 7.95 (s, 1H), 3.88 (dd, J=11.2 and 2.8 Hz, 1H), 3.78 (br d, J=11.2 Hz, 1H), 3.50-3.58 (m, 1H), 3.49 (t, J=6.0 Hz, 2H), 3.37 (br t, J=10.0 Hz, 1H), 3.11-3.17 (m, 1H), 2.94-3.04 (m, 3H), 2.79 (s, 3H), 1.98-2.08 (m, 1H), 1.94 (br, 1H), 1.77-1.85 (m, 1H); ¹⁹F NMR (376.1 MHz, CDCl₃) δ −60.47 (s, 3 F), −126.27 (dtd, J=156.0, 12.4 and 3.8 Hz, J=155.5, 12.6 and 5.5 Hz, 1 F), −141.94 (ddd, J=156.0, 13.2 and 5.3 Hz, 1 F); MS [M+H]⁺=430.1

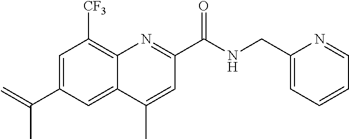

¹H-NMR (400 MHz, CDCl₃) δ 9.17 (m, 1H), 8.62 (d, 1H), 8.26 (s, 1H), 8.24 (s, 1H), 8.16 (s, 1H), 7.66 (m, 1H), 7.35 (d, 1H), 7.20 (m, 1H), 5.61 (s, 1H), 5.34 (s, 1H), 4.85 (d, 2H), 2.80 (s, 3H), 2.29 (s, 3H); ¹⁹F NMR (376.1 MHz) δ −59.99 (s); MS [M+H]⁻=386.2.

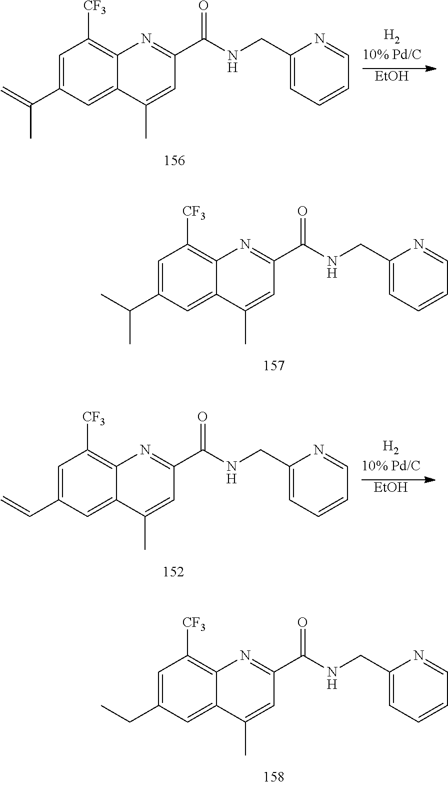

Compound 157: ¹H-NMR (400 MHz, CDCl₃) δ 9.10 (t, 1H), 8.70 (d, 1H), 8.18 (s, 1H), 7.89 (m, 2H), 7.86 (t, 1H), 7.55 (d, 1H), 7.38 (m, 1H), 4.95 (d, 2H), 3.17 (m, 1H), 2.79 (s, 3H), 1.38 & 1.37 (s, s, 6H); ¹⁹F NMR (376.1 MHz) δ −59.88 (s); MS [M+H]⁻=388.2.

Compound 158: ¹H-NMR (400 MHz, CDCl₃) δ 9.08 (t, 1H), 8.59 (d, 1H), 8.15 (s, 1H), 7.91 (d, 2H), 7.66 (m, 1H), 7.35 (d, 1H), 7.19 (t, 1H), 4.82 (d, 2H), 2.85 (m, 2H), 2.73 (s, 3H), 1.32 (t, 3H); ¹⁹F NMR (376.1 MHz) δ −60.36 (s); MS [M+H]⁻=374.2.

### Preparation of Compound 160

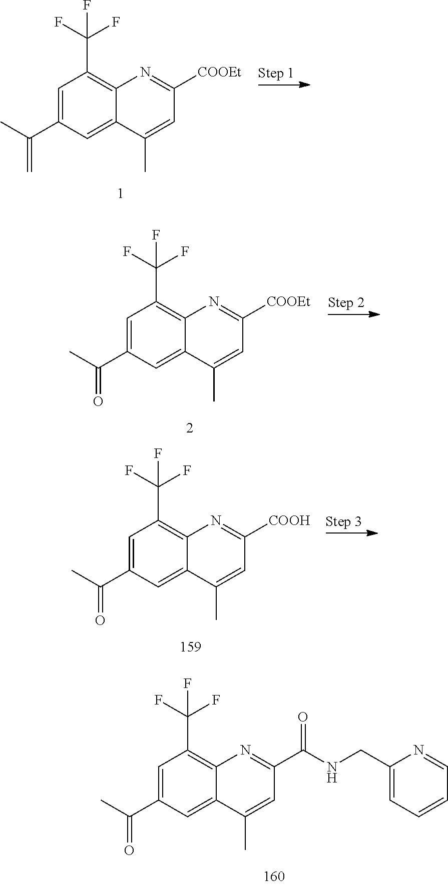

### Step 1

A solution of compound 1 (78.6 mg, 0.243 mmol) in dichloromethane (10 mL) and methanol (1 mL) was stirred at −78° C. as ozone was bubbled until the blue color appeared. After the solution was purged with oxygen until the blue color was disappeared, dimethyl sulfide (5 mL) was added and the resulting solution was stirred at it for 4.5 h. The solution was concentrated and the residue was purified by combiflash using hexanes and ethyl acetate to obtain compound 2 (72 mg, 91%). MS [M+H]⁺=326.1

### Step 2

Compound 159 (65 mg, quantitative) was prepared from in a manner similar to that described previously. MS [M+H]⁺=298.0

### Step 3

Compound 160 (25 mg, 91%) was prepared from 43 in a manner similar to that described in the synthesis of compound 14. ¹H-NMR (400 MHz, CDCl₃) δ 9.24 (br t, 1H), 8.84 (d, J=2.0 Hz, 1H), 8.64 (s, 1H), 8.64 (s, 1H), 8.32 (s, 1H), 7.68 (td, J=7.6 and 2.0 Hz, 1H), 7.35 (d, J=8.0 Hz, 1H), 7.22 (dd, J=7.8 and 5.6 Hz, 1H), 4.86 (d, J=5.6 Hz, 2H), 2.90 (s, 3H), 2.79 (s, 3H); ¹⁹F NMR (376.1 MHz, CDCl₃) δ −60.71 (s, 3 F); MS [M+H]⁺=388.1

### Preparation of Compound 161

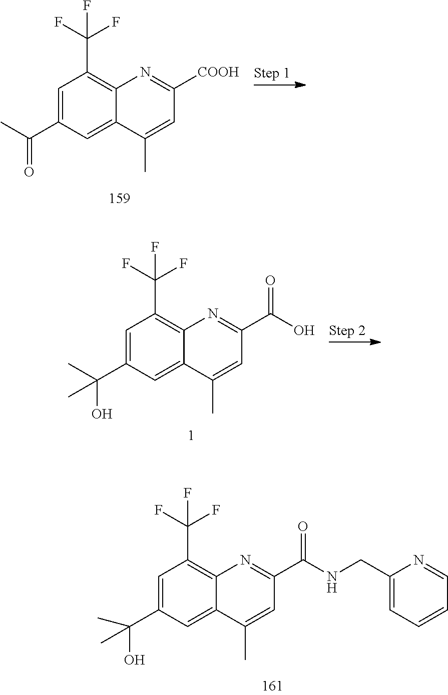

### Step 1

A solution of compound 159 (49 mg, 0.166 mmol) in THF (5 mL) was stirred at −70° C. as 3 M methylmagnesium bromide in ether (0.3 mL, 0.9 mmol) was added dropwise. After 30 min, additional 3 M methylmagnesium bromide in ether (0.3 mL, 0.9 mmol) was added and the resulting mixture was stirred for 30 min at the cold bath and then for 1 h at rt. After the mixture was quenched with 1 N HCl, the product was extracted with ethyl acetate (×2). After the extracts were washed with water (×1), combined, dried (Na₂SO₄) and concentrated, the crude compound 1 was used for the next reaction. MS [M+H]⁺=314.1

### Step 2

Compound 161 (27 mg, 37% for 2 steps) was prepared from 1 in a manner similar to that described previously. ¹H-NMR (400 MHz, CDCl₃) δ 9.19 (br t, J=5.4 Hz, 1H), 8.62 (m, 1H), 8.30 (d, J=2.0 Hz, 1H), 8.22 (d, J=2.0 Hz, 1H), 8.08 (s, 1H), 7.68 (td, J=7.6 and 2.0 Hz, 1H), 7.35 (d, J=7.6 Hz, 1H), 7.22 (dd, J=7.6 and 5.2 Hz, 1H), 4.85 (d, J=5.6 Hz, 2H), 2.73 (s, 3H), 2.7 (br, 1H), 1.72 (s, 6H); ¹⁹F NMR (376.1 MHz, CDCl₃) δ −63.03 (s, 3 F); MS [M+H]⁺=404.2

### Example 19

### Compounds 162-169

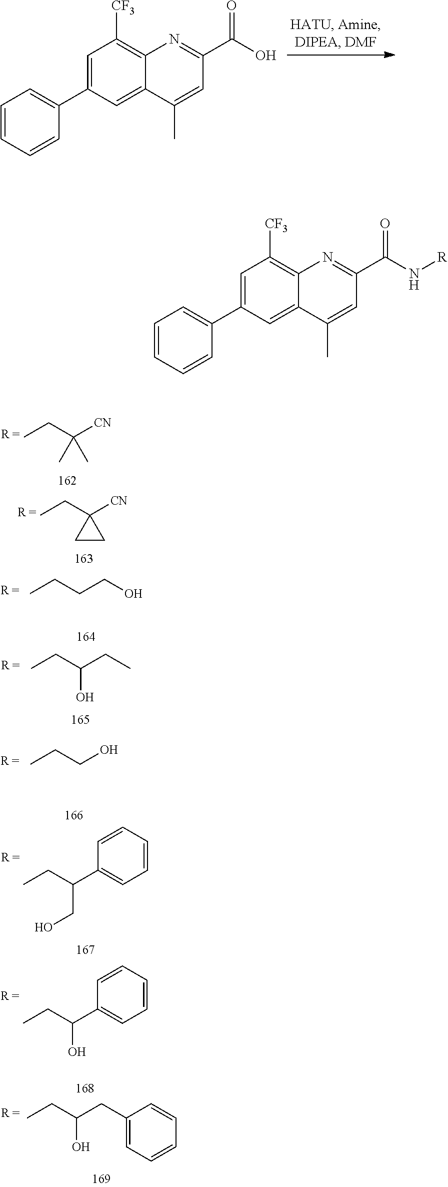

Acid (33.1 mg, 0.1 mmol) and 2,2-dimethyl-3-aminopropylnitrile hydrochloride (16.2 mg, 0.12 mmol) were dissolved in DMF (1.5 ml), followed by the addition of HATU (57 mg, 0.15 mmol), and DIPEA (38.7 mg, 0.3 mmol). The reaction was stirred at it for 4 h, and monitored by LC-MS. Reaction mixture was purified by prep-HPLC to afford light brown solid compound 162 (37.2 mg).

¹H-NMR (400 MHz, DMSO-d6) δ 8.65 (d, J=2 Hz, 1H), 8.55 (t, 1H), 8.51 (d, 1H), 8.19 (d, 1H), 7.99 (m, 1H), 7.97 (m, 1H), 7.57 (m, 2H), 7.49 (m, 1H), 3.65 (d, 2H). 2.92 (s, 3H), 1.37 (s, 6H). ¹⁹F NMR (376.1 MHz) δ −59.01 (s), 73.93 (s), MS [M+H]⁺=412.12

### Compound 163

¹H-NMR (400 MHz, DMSO-d6) δ 8.65 (s, 1H), 8.49 (m, 2H), 8.2 (s, 1H), 7.97 (m, 2H), 7.57 (m, 2H), 7.5 (m, 1H), 3.49 (m, 2H). 2.92 (s, 3H), 0.58 (d, 4H). ¹⁹F NMR (376.1 MHz) δ −58.9 (s), 74.5 (s), MS [M+H]⁺=401.09

### Compound 164

¹H-NMR (400 MHz, DMSO-d6) δ 8.63 (d, 1H), 8.55 (t, 1H), 8.48 (m, 1H), 8.16 (s, 1H), 7.97 (m, 1H), 7.95 (m, 1H), 7.57 (m, 2H), 7.5 (m, 1H), 3.53 (m, 4H). 2.91 (s, 3H), 1.72 (m, 2H). ¹⁹F NMR (376.1 MHz) δ −58.85 (s), 74.8 (s), MS [M+H]⁺=389.11

### Compound 165

¹H-NMR (400 MHz, DMSO-d6) δ 8.62 (d, 1H), 8.48 (d, 1H), 8.43 (t, 1H), 8.17 (s, 1H), 7.96 (m, 1H), 7.95 (m, 1H), 7.55 (m, 2H), 7.5 (m, 1H), 3.53 (m, 2H). 3.45 (m, 1H), 2.9 (s, 3H), 1.4 (m, 2H). 0.88 (t, 3H). ¹⁹F NMR (376.1 MHz) δ −58.45 (s), 73.8 (s), MS [M+H]⁺=403.05

### Compound 166

¹H-NMR (400 MHz, DMSO-d6) δ 8.62 (d, 1H), 8.47 (s, 1H), 8.42 (t, 1H), 8.17 (s, 1H), 7.95 (d, 2H), 7.55 (m, 2H), 7.49 (m, 1H), 3.55 (m, 2H). 3.46 (m, 2H), 2.9 (s, 3H). ¹⁹F NMR (376.1 MHz) δ −58.36 (s), 73.8 (s), MS [M+H]⁺=375.07

### Compound 167

¹H-NMR (400 MHz, DMSO-d6) δ 9.06 (d, 1H), 8.36 (s, 2H), 8.32 (s, 1H), 7.72 (m, 2H), 7.54 (m, 2H), 7.46 (m, 1H), 7.42 (m, 4H), 7.32 (m, 1H), 5.31 (m, 1H), 4.01 (m, 2H), 2.85 (s, 3H), ¹⁹F NMR (376.1 MHz) δ −59.9 (s). MS [M+H]⁺=451.04

### Compound 168

¹H-NMR (400 MHz, DMSO-d6) δ 8.6 (t, 1H), 8.35 (m, 2H), 8.24 (s, 1H), 7.7 (m, 2H), 7.54 (m, 2H), 7.47 (m, 3H), 7.38 (m, 2H), 7.35 (m, 1H), 5.02 (m, 1H), 3.91 (m, 1H), 3.74 (m, 1H), 2.85 (s, 3H). ¹⁹F NMR (376.1 MHz) δ −59.03 (s). MS [M+H]⁺=451.01

### Compound 169

¹H-NMR (400 MHz, DMSO-d6) δ 8.49 (m, 1H), 8.35 (m, 2H), 8.2 (s, 1H), 7.7 (m, 2H), 7.54 (m, 2H), 7.46 (m, 1H), 7.3 (m, 4H), 7.2 (m, 1H), 4.39 (m, 1H), 3.86 (m, 1H), 3.76 (m, 1H), 3.08 (m, 1H), 2.98 (m, 1H), 2.83 (s, 3H). ¹⁹F NMR (376.1 MHz) δ −59.76 (s). MS [M+H]⁺=465.09

### Compound 170-171

Compounds 170 and 171 were obtained from compound 169 by de-hydration.

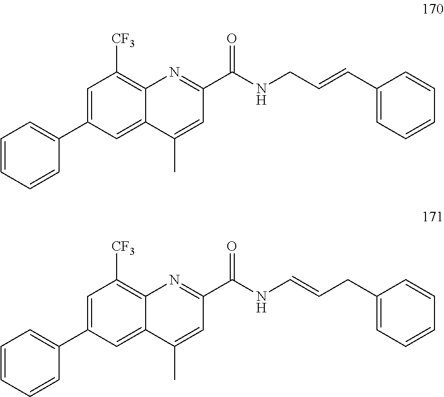

### Compound 170

¹H-NMR (400 MHz, CDCl3) δ 8.42 (m, 1H), 8.37 (m, 1H), 8.35 (m, 1H), 8.28 (m, 1H), 7.73 (m, 1H), 7.71 (m, 1H), 7.53 (m, 2H), 7.46 (m, 1H), 7.4 (m, 3H), 7.3 (m, 2H), 6.66 (m, 1H), 6.35 (m, 1H), 4.34 (m, 2H), 2.86 (s, 3H). ¹⁹F NMR (376.1 MHz) δ −59.83 (s). MS [M+H]⁺=446.97.

### Compound 171

¹H-NMR (400 MHz, CDCl3) δ 8.37 (m, 3H), 8.27 (m, 1H), 7.98 (m, 1H), 7.72 (m, 2H), 7.53 (m, 2H), 7.48 (m, 1H), 7.31 (m, 5H), 7.03 (m, 1H), 5.15 (m, 1H), 3.61 (m, 2H), 2.87 (s, 3H). ¹⁹F NMR (376.1 MHz) δ −59.91 (s). MS [M+H]⁺=447.12.

### Compound 172

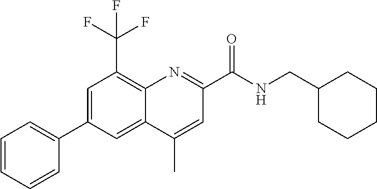

¹H-NMR (400 MHz, CCl3H-d) δ 8.33 (m, 2H), 8.22 (s, 1H), 7.71 (m, 2H), 7.53 (m, 2H), 7.43 (m, 1H), 3.42 (m, 1H), 3.38 (m, 2H), 2.84 (s, 3H) 1.82-1.04 (m, 11H); ¹⁹F NMR (400 MHz, CCl₃H-d) δ −60.06 (s); MS [M+H]⁺=427.

### Example 20

### Preparation of Compound 173

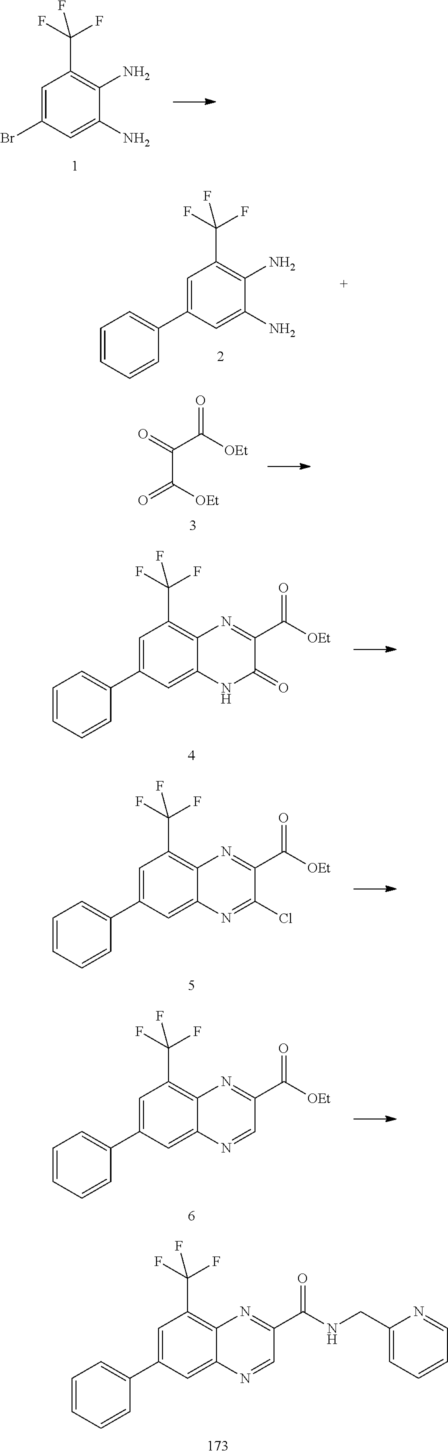

### Step 1

### 5-Bromo-3-(trifluoromethyl)benzene-1,2-diamine 1 (998 mg, 3.92 mmol), phenylboronic acid (575 mg, 4.71 mmol) and palladium tetrakis(triphenylphosphine)palladium(0) (228 mg, 0.197 mmol) in dioxane (10 mL) and 1 M K₃PO₄ (5 mL) was heated at 140° C. in a microwave reactor for 10 min. The reaction mixture was diluted with ethyl acetate and washed with water (×2). After the aqueous fractions were extracted with ethyl acetate (×1), the combined organic fractions were dried (Na₂SO₄) and concentrated. The residue was purified by combiflash using hexane and ethyl acetate as eluents to obtain 570 mg (58%) of compound 2.

### Step 2

A solution of compound 2 (450 mg, 1.78 mmol) and diethyl ketomalonate 3 (0.33 mL, 2.14 mmol) in ethanol (7 mL) was refluxed at 85° C. oil bath for 3 h. After the resulting mixture was concentrated, the residue was dissolved in hot ethyl acetate and adsorbed on silicagel to purify by combiflash using ethyl acetate and hexane as eluents to obtain 282 mg (44%) of 12 and 223 mg (35%) of 4. MS [M+H]⁺=363.0

A mixture of compound 4 (137 mg, 0.378 mmol) and dimethylaniline (24 uL, 0.189 mmol) in POCl₃ (5 mL) was refluxed for 3.5 h and concentrated. After the residue was treated with ice followed by aq. NaHCO₃, the product was extracted with ethyl acetate (2×30 mL). The extracts were washed with water (×1) combined, dried (Na₂SO₄) and concentrated. The product was purified by combiflash using ethyl acetate and hexane as eluents to obtain 123 mg (85%) of compound 5. MS [M+H]⁺=381.0 (very weak)

A mixture of compound 5 (123 mg, 0.323 mmol), sodium acetate (114 mg, 1.39 mmol), and 10% Pd/C (11.5 mg) in DMF (3 mL) was stirred under H2 atmosphere at rt for 1 h and then added additional 10% Pd/C (20.4 mg) before stirring at rt for 2.5 h. After the mixture was filtered through a celite pad, the filtrate was concentrated. The residue was dissolved in ethyl acetate, washed with water (×1), dried (Na₂SO₄) and concentrated with small amount of silica gel. The adsorbed product was purified by combiflash using hexane and ethyl acetate as eluents to obtain 73.3 mg (66%) of compound 6. MS [M+H]⁺=347.0

Compound 173 (22 mg, 90% for 2 steps) was prepared from compound 6 (21 mg, 0.060 mmol) in a manner similar to that previously. ¹H-NMR (400 MHz, CDCl₃) δ 9.79 (s, 1H), 9.08 (br, 1H), 8.66 (d, J=4.0 Hz, 1H), 8.56 (s, 1H), 8.47 (s, 1H), 7.74-7.83 (m, 3H), 7.58 (t, J=7.6 Hz, 2H), 7.51 (t, J=7.6 Hz, 1H), 7.46 (d, J=7.6 Hz, 1H), 4.93 (d, J=5.2 Hz, 2H); ¹⁹F NMR (376.1 MHz, CDCl₃) δ −59.63 (s, 3 F); MS [M+H]⁺=409.2

### Example 21

### Preparation of Compound 174

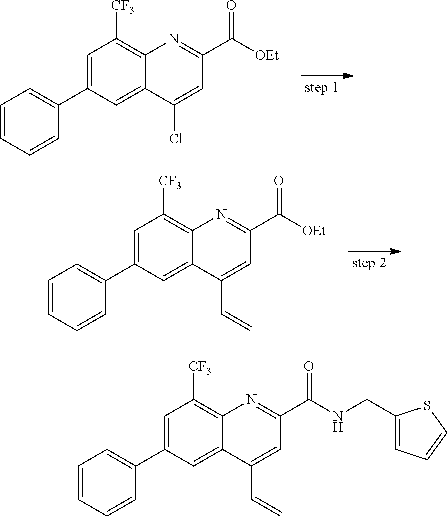

### Step 1

A solution of starting material (50 mg, 0.12 mmol), TEA (0.17 μL, 0.12 mmol), potassium trifluorovinylborate (24.1 mg, 0.12 eq) and 1,1′-Bis(diphenylphosphino)ferrocene-palladium(II)dichloride dichloromethane complex (9.6 mg, 0.01 mmol) in 2.5 mL EtOH was heated at 70° C. for 1.5 h. The reaction mixture was cooled to it and diluted with 50 mL EtOAc and 50 mL phosphate buffer (pH 3.0). The organic layer was separated, dried with sodium sulfate, filtered thru a silica plug and concentrated in vacuo to provide the desired product (63 mg, 126%) as a brown oil contaminated with the EtOH adduct. MS [M+H]⁺=416.1, LCMS rt=2.84 min.

### Step 2

The crude product from step 1 was taken up in 2.5 mL THF and treated with LiOH (240 uL, 0.24 mmol, 1M aqueous) and the solution allowed to stir at it for 3 h. The reaction was treated with HCl (240 uL, 0.24 mmol, 1 M aqueous) then diluted with dioxane (25 mL) and concentrated in vacuo. The dilution and concentration from dioxane was repeated twice. The residue was taken up in 3 mL DMF and treated with py-BOP (94 mg, 0.18 mmol), NMM (66 uL, 0.6 mmol) and aminomethylthiophene (18 uL, 0.18 mmol). After 15 min stirring, the crude reaction mixture was purified by RP-HPLC to provide the desired product (11.3 mg, 21% yield, 2 steps).

¹H-NMR (400 MHz, DMSO) δ 8.86 (s, 1H), 8.57 (s, 1H), 8.46 (s, 1H), 8.02 (d, J=7 Hz, 2H), 7.64-7.56 (m, 2H), 7.55 (d, J=7 Hz, 1H), 7.47 (dd, J=5, 1 Hz, 1H), 7.15 (d, J=3 Hz, 1H), 7.05-7.02 (m, 1H), 6.37 (d, J=18 Hz, 1H), 5.90 (d, J=12 Hz, 1H), 4.86 (d, J=7 Hz, 2H); MS [M+H]⁺=439.0

### Example 23

### Preparation of Compound 175

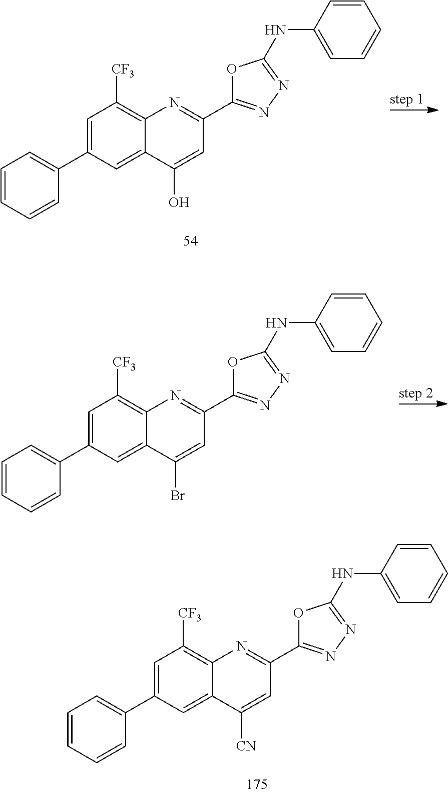

A suspension of compound 54 (255 mg, 0.57 mmol) was taken up in 2 mL DCE and POBr₃ (585 mg, 2 mmol) was added. The mixture was heated to 80° C. for 1 h, then cooled to rt. The reaction was quenched with water and EtOAc and the mixture stirred vigorously for 15 min. The organic layer was separated, washed with sat. NaHCO₃, dried with sodium sulfate and concentrated in vacuo. The solid residue was triturated with Et₂O to provide the desired product (237 mg, 81% yield) as an orange solid. ¹H-NMR (400 MHz, DMSO-d₆) δ 11.10 (s, 1H), 8.69 (s, 1H), 8.64 (d, J=3 Hz, 2H), 7.99 (d, J=7 Hz, 2H), 7.65 (d, J=8 Hz, 2H), 7.64 (dd, J=8, 7 Hz, 2H), 7.58 (d, J=7 Hz, 1H), 7.44 (ap t, J=8 Hz, 2H), 7.11 (ap t, J=8 Hz, 1H); MS [M+H]⁺=511.1, 513.1, LCMS rt=2.82 min.

### Step 2

A mixture of bromide (100 mg, 0.195 mmol) and copper(I) cyanide (87 mg, 0.98 mmol) in 1.5 mL DMSO was heated under μ-wave radiation at 160° C. for 60 min. The mixture was diluted with EtOAc and 1:1 NH₃:NH₄Cl. The organic layer was separated and washed with the ammonium chloride buffer (2×) and brine. The organic layer was dried with sodium sulfate and concentrated in vacuo. The residue was triturated with Et₂O and water to provide the desired product 175 (85 mg, 95% yield) contaminated with 10% of the starting bromide.

¹H-NMR (400 MHz, DMSO-d₆) δ 8.97 (s, 1H), 8.71 (s, 1H), 8.56 (s, 1H), 8.03 (d, J=7 Hz, 2H), 7.76 (d, J=8 Hz, 2H), 7.66 (dd, J=8, 7 Hz, 2H), 7.60 (d, J=7 Hz, 1H), 7.44 (ap t, J=8 Hz, 2H), 7.11 (ap t, J=7 Hz, 1H); MS [M+H]⁺=458.1; LCMS rt=2.83.

### Example 23

The following compounds were made from compound 76 by the standard cross coupling reactions with appropriate reagents:

### Preparation of Compound 176

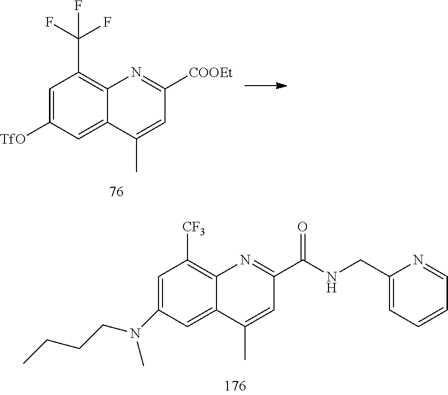

¹H-NMR (400 MHz, CDCl3) δ 8.96 (m, 1H), 8.79 (m, 1H), 8.30 (m, 1H), 7.94 (m, 2H), 7.75 (m, 1H), 7.67 (s, 1H), 6.94 (m, 1H), 5.09 (d, 2H), 3.48 (m, 2H), 3.10 (s, 3H), 2.61 (s, 3H), 1.61 (m, 2H), 1.37 (m, 2H), 0.96 (m, 3H); ¹⁹F NMR (376.1 MHz) δ −60.10 (s); MS [M−H]⁺=431.2.

### Compound 177

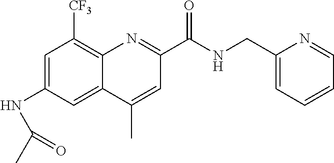

¹H-NMR (400 MHz, DMSO) δ 10.63 (s, 1H), 9.06 (t, 1H), 8.67 (d, 1H), 8.61 (d, 1H), 8.44 (d, 1H), 8.10 (s, 1H), 7.96 (t, 1H), 7.52 (d, 1H), 7.45 (d, 1H), 4.76 (d, 2H), 2.72 (s, 3H), 2.13 (s, 3H); ¹⁹F NMR (376.1 MHz) δ −59.14 (s), −75.16 (s); MS [M−H]⁺=403.14.

### Compound 178

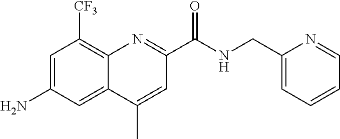

¹H-NMR (400 MHz, DMSO) δ 8.91 (t, 1H), 8.63 (d, 1H), 8.03 (t, 1H), 7.9 (s, 1H), 7.68 (d, 1H), 7.56 (d, 1H), 7.51 (t, 1H), 7.14 (d, 1H), 4.76 (d, 2H), 2.58 (s, 3H); ¹⁹F NMR (376.1 MHz) δ −59.16 (s), −75.26 (s); MS [M−H]⁺=361.17.

### Compound 179

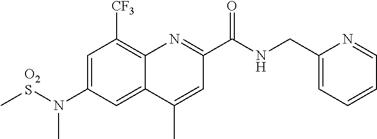

¹H-NMR (400 MHz, DMSO) δ 9.09 (t, 1H), 8.55 (d, 1H), 8.14 (s, 1H), 8.09 (s, 1H), 7.83 (t, 1H), 7.41 (d, 1H), 7.33 (t, 1H), 4.72 (d, 2H), 3.57 (s, 3H), 3.17 (s, 3H), 2.46 (s, 3H); ¹⁹F NMR (376.1 MHz) δ −59.23 (s), −75.16 (s); MS [M−H]⁺=453.46.

### Compound 180

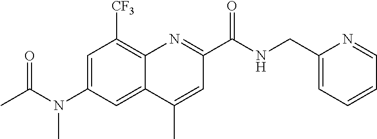

¹H-NMR (400 MHz, CD3OD) δ 8.61 (d, 1H), 8.29 (m, 2H), 8.13 (m, 2H), 7.83 (d, 1H), 7.71 (t, 1H), 5.20 (d, 2H), 3.34 (s, 3H), 2.77 (s. 3H), 1.92 (s, 3H); ¹⁹F NMR (376.1 MHz) δ −61.67 (s), −77.89 (s); MS [M−H]⁺=417.16.

### Compound 181

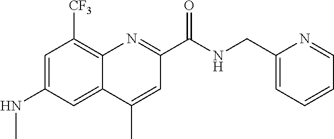

¹H-NMR (400 MHz, DMSO) δ 8.92 (t, 1H), 8.60 (d, 1H), 7.93 (m, 2H), 7.69 (s, 1H), 7.49 (d, 1H), 7.43 (t, 1H), 6.83 (d, 1H), 4.74 (d, 2H), 2.85 (s, 3H), 2.64 (s, 3H); ¹⁹F NMR (376.1 MHz) δ −59.33 (s), −75.08 (s); MS [M−H]⁺=375.12.

### Example 24

The following compounds were prepared from compound 75 by standard alkylation and coupling reactions with amines.

### Compound 182

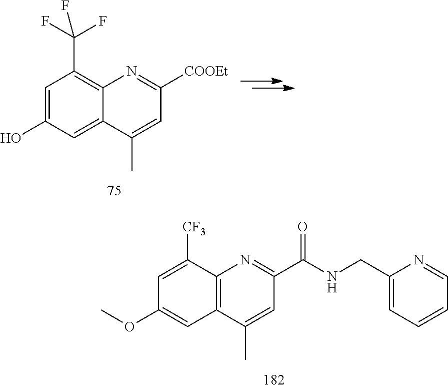

¹H-NMR (400 MHz, DMSO) δ 9.06 (dd, 1H), 8.63 (d, 1H), 8.11 (s, 1H), 7.98 (m, 1H), 7.87 (s, 1H), 7.66 (dd, 1H), 7.55 (m, 2H), 7.47 (m, 1H), 4.78 (d, 2H), 4.02 (s, 3H), 2.79 (s, 3H); ¹⁹F NMR (376.1 MHz) δ −59.17 (s); MS [M−H]⁺=376.1.

### Compound 183

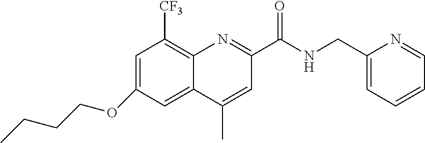

¹H-NMR (400 MHz, DMSO) δ 9.05 (t, 1H), 8.61 (d, 1H), 8.07 (s, 2H), 7.95 (t, 1H), 7.82 (d, 1H), 7.62 (m, 1H), 7.52 (m, 1H), 7.43 (t, 1H), 4.75 (d, 2H), 4.21 (t, 1H), 2.75 (s, 3H), 1.78 (m, 2H), 1.46 (m, 2H), 0.94 (m, 2H); ¹⁹F NMR (376.1 MHz) δ −58.70 (s); MS [M−H]⁺=418.2.

### Compound 184

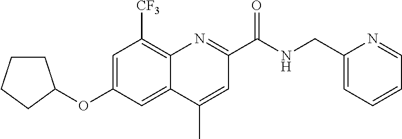

¹H-NMR (400 MHz, DMSO) δ 9.04 (t, 1H), 8.64 (dd, 1H), 8.02 (m, 2H), 7.77 (d, 1H), 7.59 (m, 2H), 7.52 (m, 1H), 5.14 (m, 1H), 4.78 (d, 2H), 2.74 (s, 3H), 1.79-1.58 (m, 8H); ¹⁹F NMR (376.1 MHz) δ −58.71 (s); MS [M−H]⁺=430.2.

### Compound 187

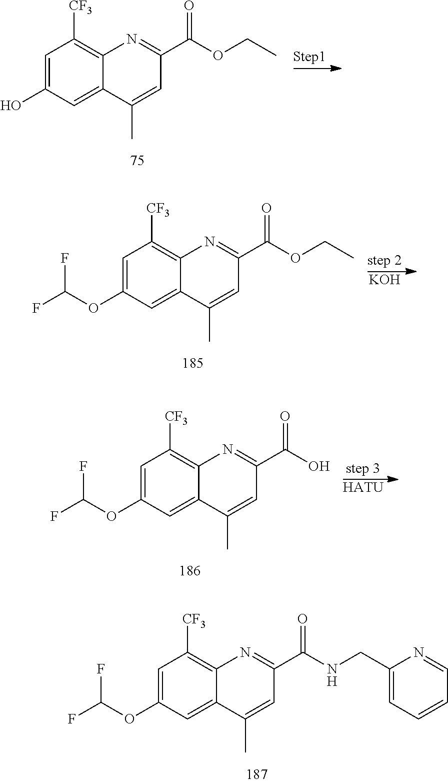

### Step 1

### 6-Hydroxy-4-methyl-8-trifluoromethyl-quinoline-2-carboxylic acid ethyl ester 75 (0.1 g, 0.33 mmol) was dissolved in Dioxane (2 ml) and followed by the addition of NaOH (1N, 2 ml) in a sealed tube (10 ml size). The solution was cooled to −41° C., and chlorodifluoromethane was bubbled in the solution for 3 min, then the tube was capped tightly and heated at 60° C. for 3 hours. The reaction was then cooled to room temperature and pH was adjusted to 7 by 9 N HCl and extracted with EtOAc (5 ml, 3×). Organic phases were combined and dried with sodium sulfate. After removal of the solvent in vacuo, crude compound 185 was obtained and no further purification was performed.

### Step 2 and 3

The procedures were described previously.

Compound 187: ¹H-NMR (400 MHz, DMSO) δ 9.08 (t, 1H), 8.56 (d, 1H), 8.15 (s, 1H), 8.11-8.07 (dd, 2H), 7.88 (t, 1H), 7.46 (d, 1H), 7.37 (d, 1H), 7.39 (t, 1H), 4.78 (d, 2H), 2.76 (s, 3H); ¹⁹F NMR (376.1 MHz) δ −59.14 (s), −75.08 (s), −83.86 (d); MS [M−H]⁺=412.08.

### Preparation of Compound 189

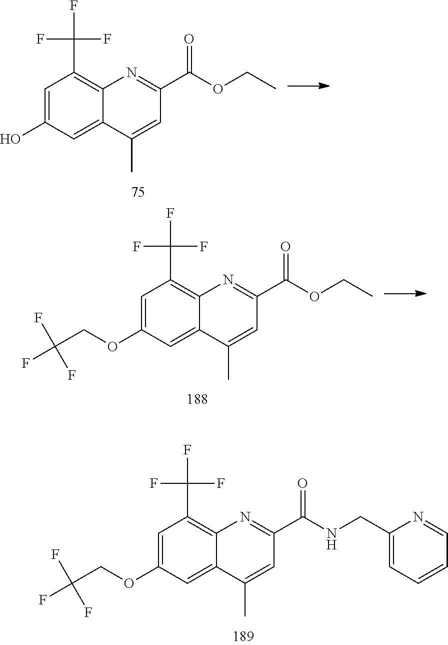

### Step 1

Potassium carbonate (388 mg, 2.81 mmol)), compound 75 (330 mg, 1.1 mmol), 2-iodo-1,1,1-trifluoroethane (462 mg, 2.2 mmol) and DMF (2.5 mL) were heated in a microwave until no further reaction was noticed (HPLC analysis). The reaction was diluted into ethyl acetate (50 mL) and water (25 mL). Ethyl acetate (3×˜12 mL) was used to extract the aqueous phase. The combined organic phases were washed with 5% aqueous LiCl and brine before drying (Na₂SO₄), filtering, and evaporation in vacuo at 30° C. Purification was accomplished via flash chromatography (silica gel) affording compound 188 (194 mg).

¹H NMR (400 MHz, cdcl₃) δ 8.08 (s, 1H), 7.85 (d, J=2.7 Hz, 1H), 7.42 (d, J=2.6 Hz, 1H), 4.63-4.43 (m, 4H), 2.74 (s, 3H), 1.46 (t, J=7.1 Hz, 3H); ¹⁹F NMR (376 MHz, cdcl₃) δ −60.89 (s), −74.06 (t, J=7.8 Hz); MS [M+H]⁺=354.00.

### Step 2

Compound 188 (7.6 mg, 0.256 mmol), 2-aminomethylpyridine (157 μL, 1.53 mmol) and DMF (1 mL) were heated in a microwave reactor (140° C., 20 min; 180° C., 30 min; 180° C., 2 h; 200° C., 1 h). Amine (0.1 mL, 0.97 mmol) was added to the reaction and heating continued (180° C., 2 h). Isolation and purification were accomplished via preparative HPLC affording compound 189 (59.6 mg).

¹H NMR (400 MHz, dmso) δ 9.07 (t, J=5.5 Hz, 1H), 8.59 (d, J=4.8 Hz, 1H), 8.13 (s, 1H), 8.03-7.85 (m, 3H), 7.50 (s, 1H), 7.42 (dd, J=19.7, 13.6 Hz, 1H), 5.10 (q, J=8.8 Hz, 2H), 4.75 (d, J=5.6 Hz, 2H), 2.79 (s, 3H); ¹⁹F NMR (376 MHz, dmso) δ −59.15 (s), −72.85 (t, J=8.8 Hz), −75.07 (s); MS [M+H]⁺=444.21.

### Compound 190

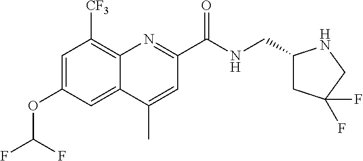

¹H-NMR (400 MHz, CH₃OH-d₄) δ 8.23 (s, 1H), 8.09 (s, 1H), 8.01 (s, 1H), 7.45-6.95 (m, 1H), 3.63 (m, 3H), 3.25 (m, 2H), 2.82 (s, 3H), 2.45 (m, 1H), 2.16 (m, 1H); ¹⁹F NMR (400 MHz, CH₃OH-d₄) δ −61.95 (s, 3 F), −85.20 (d, 2 F), −97.23-100.44 (m, 2 F); MS [M+H]⁺=440.

### Compound 191

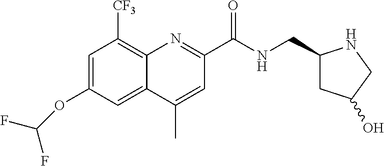

¹H-NMR (400 MHz, CH₃OH-d₄) δ 8.26 (s, 1H), 8.11 (s, 1H), 8.03 (s, 1H), 7.44-6.95 (m, 1H), 4.60 (m, 1H), 4.20 (m, 1H), 3.85 (m, 1H), 3.52 (m, 1H), 3.28 (m, 1H), 3.20 (m, 1H), 2.82 (s, 3H), 2.22 (m, 1H), 1.72 (m, 1H); ¹⁹F NMR (400 MHz, CH₃OH-d₄) δ −61.85 (s, 3 F), −85.50 (d, 2 F); MS [M+H]⁺=420.

### Preparation of Compound 194

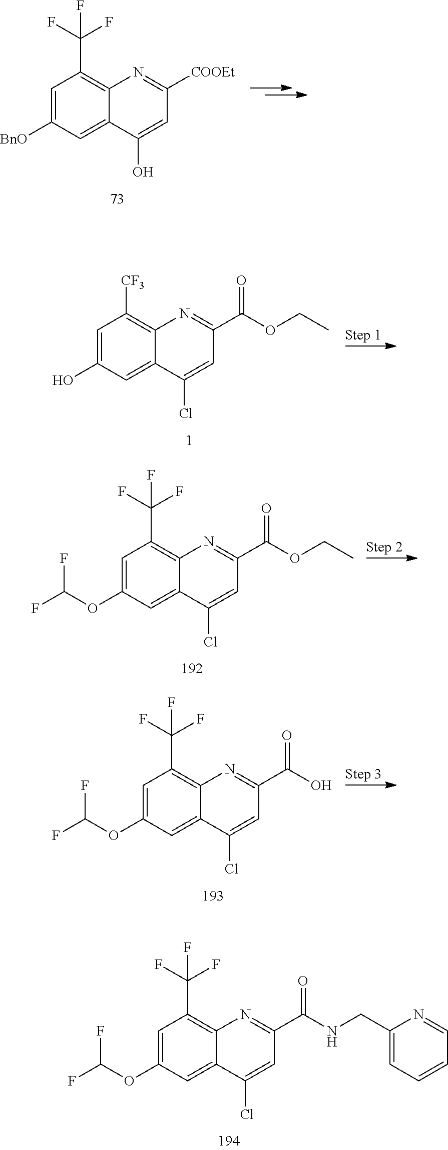

### Step 1

Phenol (1.6 g, 5 mmol, prepared from 73) and K₂CO₃ (25 g, 180 mmol) dissolved in mixture of acetonitril (18 ml) and water (18 ml) was added 1-chloro-1,1-difluoroacetophone (5 g, 25 mmol) at rt. After 4 h heating at 80° C. The reaction mixture was poured into saturated water solution of NaHCO₃ and diluted with EtOAc, washed with sat'd NaHCO₃ and brine. The organic layer was dried (Na₂SO₄) and concentrated. The crude product was purified by flash chromatography on silica gel with EA/Hex to give 780 mg difluoromethyl phenol ether 192. ¹H-NMR (400 MHz, DMSO-d6) δ 8.34 (s, 1H), 8.1 (m, 1H), 8.97 (m, 1H), 6.74 (t, 1H), 4.5 (q, 2H), 1.47 (t, 3H). ¹⁹F NMR (376.1 MHz) δ −60.88 (s), −83 (d). MS [M+H]⁺=370.08.

### Step 2

Ethyl ester (208 mg, 0.56 mmol) dissolved MeOH (5 ml) was added 2N LiOH in water (0.5 ml, 1 mmol) at RT. After 4 h, The reaction mixture was poured into 1N HCl solution (20 ml), and diluted with EtOAc, washed with brine. The organic layer was dried (Na₂SO₄) and concentrated to give crude acid 194. MS [M+H]⁺=338.13.

### Step 3

Acid (67 mg, 2 mmol), 1-aminomethylpyridium (27 mg, 0.25 mmol) and DIPEA (51.6 mg, 0.4 mmol) dissolved in DMF (3 ml) was added HATU (152 mg, 0.4 mmol) at RT. After 2 h, the reaction mixture was subject to prepare HPLC purification to give 61 mg of compound 194. ¹H-NMR (400 MHz, DMSO-d6) δ 9.14 (s, 1H), 8.63 (d, 1H), 8.52 (s, 1H), 8.14 (d, 1H), 7.97 (d, 1H), 7.68 (m, 1H), 7.34 (m, 1H), 7.22 (m, 1H), 6.73 (t, 1H), 4.84 (m, 2H). ¹⁹F NMR (376.1 MHz) δ −60.77 (s), −86.9 (d). MS [M+H]⁺=432.29.

### Preparation of Compound 195

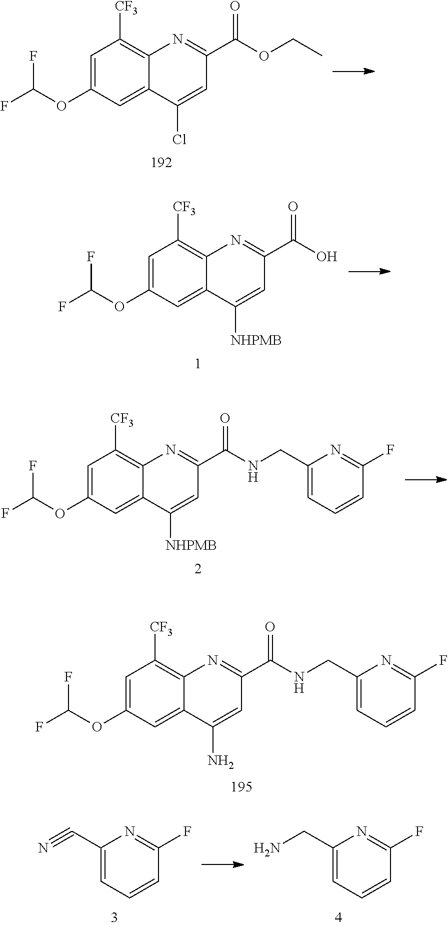

Compound 192 (0.2 g, 0.54 mmol), NH₂PMB (0.15 g, 1.08 mmol), and Cs₂CO₃ (0.7 g, 2.1 mmol) were dissolved in Dioxane (3 ml). the mixture solution was purged with N₂ three time and then followed by the addition of Pd₂(dba)₃ (0.026 g, 0.027 mmol). The resulting solution was purged with N₂ two more times and stirred at 95° C. for 4 hours. The reaction mixture was diluted with EtOAc and washed with H₂O (5×, 40 ml) and desired product went into aqueous phase and impurities stayed in organic phase. Aqueous phase was concentrated down to 30 ml and acidified to pH 5 by HCl (Con.), and was extracted by EtOAc (3×, 30 ml). The total organic phase were combined and dried with sodium sulfate. After removal of the solvent in vacuo, compound 1 was obtained (120 mg, 50%).

Other procedures were described previously.

6-Fluoro-pyridine-2-carbonitrile 3 (0.2 g, 1.64 mmol) was dissolved in MeOH (4 ml), and followed by the addition of Pd/C (10% wet) (0.05 g) and HCl (Con.) (1 ml). The resulting reaction mixture was purged with H₂ five times and stirred at room temperature 4 hours. The reaction mixture was filtered through a pile of celite pad and the filtration was stripped off to obtain the crude product in light yellow solid form. No further purification was performed.

Amine was coupled with 4 to obtain 2 which was deprotected to afford compound 195:

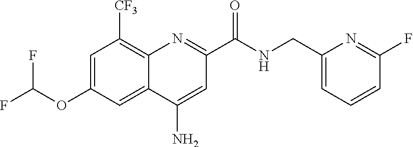

¹H-NMR (400 MHz, CD3OD) δ 8.11 (d, 1H), 7.92-7.84 (m, 2H), 7.42 (s, 1H), 7.29 (dd, 1H), 6.93 (dd, 1H), 4.69 (d, 2H), 3.33 (s, 1H); ¹⁹F NMR (376.1 MHz) δ −62.38 (s), −70.60 (d), −84.65 (d); MS [M−H]⁺=431.29

### Preparation of Compounds 196 and 197

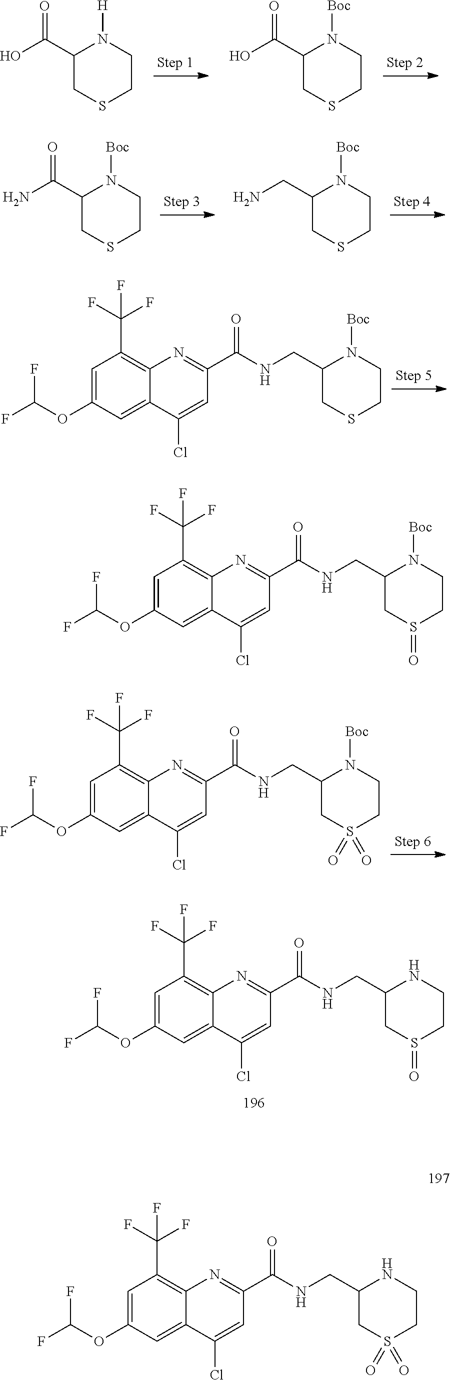

### Step 1

Thiomorpholine carboxylic acid hydrochloride (1.5 g, 8.2 mmol) and TEA (2.89 g, 28.6 mmol) dissolved in DCM (50 ml) was added (Boc)₂O (2.7 g, 12.3 mmol) at RT. After 4 h, the reaction mixture was poured into 1N HCl water solution and diluted with EtOAc, washed with brine. The organic layer was dried (Na₂SO₄) and concentrated to give 3.1 g Boc protected thiomorpholine carboxylic acid. ¹H-NMR (400 MHz, CDCl₃) δ 5.2 (d, 1H), 4.3 (dd, 1H), 3.2 (m, 1H), 3.1 (t, 1H), 2.9 (dd, 1H), 2.71 (t, 1H), 2.46 (m, 1H), 1.48 (s, 9H).

### Step 2

Boc protected thiomorpholine carboxylic acid (3.1 g, 13 mmol), HOBt (2.1 g, 16 mmol) and EDCl hydrochloride (3.7 g, 20 mmol) dissolved in DMF (20 ml) was added 28% of ammonium hydroxide solution (4.5 g, 130 mmol) at RT. After 4 h, the reaction mixture was poured into saturated water solution of NaHCO₃ and diluted with EtOAc, washed with sat'd NaHCO₃ and brine. The organic layer was dried (Na₂SO₄) and concentrated. The crude product was purified by flash chromatography on silica gel with EA/Hex to give 1.23 g of amide. ¹H-NMR (400 MHz, CDCl₃) δ 5.2 (d, 1H), 6.15 (br., 1H), 5.68 (br., 1H), 5.02 (br., 1H), 4.25 (br., 1H), 3.16 (d, 1H), 2.8 (m, 1H), 2.7 (t, 1H), 2.4 (d, 1H), 1.47 (s, 9H).

### Step 3

Amide (890 mg, 3.6 mmol) dissolved in THF (20 ml) was added 1N solution of BH₃ in THF (15 ml) RT. After reflux 4 h, the reaction mixture was quenched with MeOH, then the mixture was poured into saturated water solution of NaHCO₃ and diluted with EtOAc, washed with sat'd NaHCO₃ and brine. The organic layer was dried (Na₂SO₄) and concentrated to give 547 mg crude amine.

### Step 4

Acid (100 mg, 0.22 mmol), crude amine (70 mg) and DIPEA (77.4 mg, 0.6 mmol) dissolved in DMF (5 ml) was added HATU (171 mg, 0.45 mmol) at RT. After 2 h, the reaction mixture was poured into saturated water solution of NaHCO₃ and diluted with EtOAc, washed with sat'd NaHCO₃ and brine. The organic layer was dried (Na₂SO₄) and concentrated. The crude product was purified by flash chromatography on silica gel with EA/Hex to give coupling product.

### Step 5

Coupling product (130 mg, 0.23 mmol) dissolved in MeOH (20 ml) was added oxone (431 mg, 0.7 mmol) at RT. After 2 h, the reaction was quenched with 10% of Na₂S₂O₃ in sat'd NaHCO₃ water solution. The reaction mixture was poured into saturated water solution of NaHCO₃ and diluted with EtOAc, washed with sat'd NaHCO₃ and brine. The organic layer was dried (Na₂SO₄) and concentrated to give mixture of sulfoxide and sulfone.

### Step 6

The mixture of sulfoxide and sulfone dissolved in DCM (5 ml) was added TFA (2 ml) at RT. After 2 h, the solvent was removed, the residue was subject to prepare HPLC purification to give 4.2 mg of compound 196 and 72.5 mg of compound 197.

### Compound 196

¹H-NMR (400 MHz, DMSO-d6) δ 8.75 (t, 1H), 8.48 (s, 1H), 8.26 (d, 1H), 8.12 (d, 1H), 7.23 (t, 1H), 4.24 (m, 1H), 3.87 (m, 2H), 3.55 (m, 1H), 3.3 (m, 3H), 3.2 (m, 1H), 2.99 (m, 2H). ¹⁹F NMR (376.1 MHz) δ −61.85 (s), −77.64 (s), −86 (d). MS [M+H]⁺=472.15.

### Compound 197

¹H-NMR (400 MHz, DMSO-d6) δ 8.63 (t, 1H), 8.4 (s, 1H), 8.15 (d, 1H), 8.05 (d, 1H), 7.15 (t, 1H), 3.94 (m, 1H), 3.75 (m, 3H), 3.46 (m, 2H), 3.31 (m, 2H), 3.22 (m, 2H). ¹⁹F NMR (376.1 MHz) δ −61.87 (s), −77.68 (s), −86 (d). MS [M+H]⁺=488.08.

### Compound 198

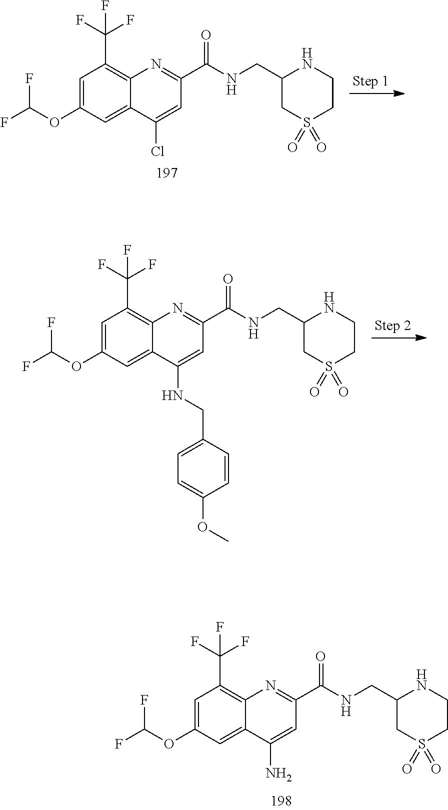

### Step 1

Compound 197 (57 mg, 0.11 mmol), p-Methoxylbenzylamine (69 mg, 0.5 mmol), tris(dibenzylidenacetone)dipalladium(0) chloroform adduct (10.4 mg, 0.01 mmol) and (Cs)₂CO₃ (163 mg, 0.5 mmol) in dioxane (5 ml) was added 2-dicyclohexylphosphino-2′-(N,N-dimethylamino)biphenyl (7.88 mg, 0.02 mmol) at RT. After heating to 100° C. for 4 h, the reaction mixture was poured into saturated water solution of NaHCO₃ and diluted with EtOAc, washed with sat'd NaHCO₃ and brine. The organic layer was dried (Na₂SO₄) and concentrated to give crude coupling product.

### Step 2

Coupling product dissolved in TFA (2 ml) was added MsOH (1 ml) at RT. After 2 h, the TFA and MsOH was removed under vacuum. The residue was subjected to prepare HPLC purification to give 22 mg of compound 198. ¹H-NMR (400 MHz, DMSO-d6) δ 8.06 (d, 1H), 7.8 (d, 1H), 7.38 (s, 1H), 6.93 (t, 1H), 3.96 (m, 1H), 3.72 (m, 2H), 3.49 (m, 2H), 3.32 (m, 2H), 3.21 (m, 3H). ¹⁹F NMR (376.1 MHz) δ −62.25 (s), −77.78 (s), −84.8 (d). MS [M+H]⁺=469.06.

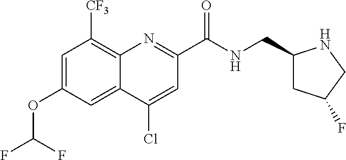

¹H-NMR (400 MHz, CH₃OH-d₄) δ 8.51 (s, 1H), 8.22 (s, 1H), 8.18 (s, 1H), 7.42-7.05 (m, 1H), 5.51-5.38 (m, 1H), 4.18 (m, 1H), 3.88 (m, 2H), 3.78-3.45 (m, 2H), 2.60 (m, 1H), 2.22 (m, 1H); ¹⁹F NMR (400 MHz, CH₃OH-d₄) δ −61.90 (s, 3 F), −86.10 (d, 2 F), −176.52 (m, 1 F); MS [M+H]⁺=442.

### Compound 200

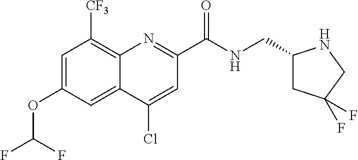

¹H-NMR (400 MHz, CH₃OH-d₄) δ 8.43 (s, 1H), 8.24 (s, 1H), 8.15 (s, 1H), 7.45-7.00 (m, 1H), 3.63 (m, 3H), 3.25 (m, 2H), 2.45 (m, 1H), 2.16 (m, 1H); ¹⁹F NMR (400 MHz, CH₃OH-d₄) δ −61.95 (s, 3F), −85.90 (d, 2 F), −97.23-100.44 (m, 2 F); MS [M+H]⁺=460.

### Preparation of Compound 202

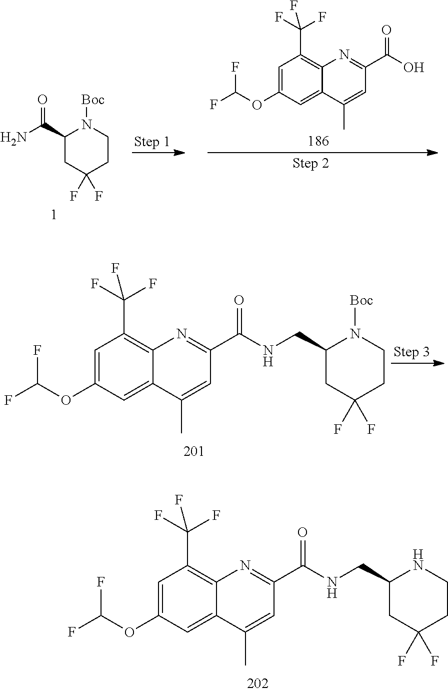

### Step 1 and Step 2

Compound 201 (41 mg, 19%) was prepared from compound 186 (50 mg, 0.086 mmol) in a manner similar to that described in the synthesis of compound 76. MS [M+H]⁺=554.0

### Step 3

Compound 202 (46 mg, quantitative) was prepared from compound 201 in a manner similar to that described previously. ¹H-NMR (400 MHz, CD₃OD) δ 8.75 (br t, J=6.4 Hz, 1H), 8.23 (s, 1H), 8.07 (d, J=2.2 Hz, 1H), 8.02 (d, J=2.2 Hz, 1H), 7.18 (t, J=73.0 Hz, 1H), 3.88 (dd, J=14.8 and 7.2 Hz, 1H), 3.77 (dd, J=14.8 and 3.2 Hz, 1H), 3.68-3.78 (m, 1H), 2.52 (m, 1H), 3.57 (dm, J=−13.6 Hz, 1H), 3.20 (td, J=13.6 and 2.8 Hz, 1H), 2.83 (s, 3H), 2.52 (br m, 1H), 2.06-2.42 (m, 3H); ¹⁹F NMR (376.1 MHz, CD₃OD) δ −61.85 (s, 3F), −77.98 (s, 6F), −85.49 (d, J=73.0 Hz, 2F), −95.46 (d, J=244.8 Hz, 1F), −103.44 (dtt, J=244.8, 32.2 and 10.7 Hz, 1F); MS [M+H]⁺=454.1

### Preparation of Compound 203

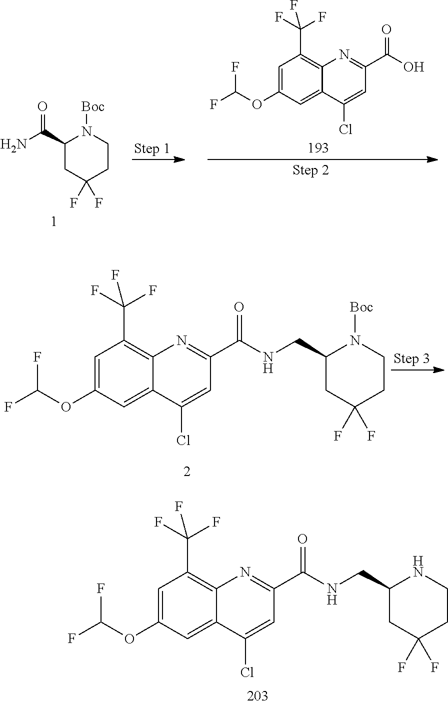

### Step 1 and Step 2

A solution of compound 1 (457 mg, 1.73 mmol) in THF (9 mL) was stirred at rt as 1.0 M borane-THF complex in THF (9 mL, 9 mmol) was added and the resulting solution was refluxed for 2 h. After cooling to rt, methanol (15 mL) was added carefully and the resulting solution was concentrated. The residue was dissolved in ether, washed with 1N NaOH (×1), and water (×1). After the organic fraction was dried (MgSO₄) and concentrated, the residue was used for the next reaction. A solution of the crude amine, compound 193 (506 mg, 1.480 mmol), and HATU (850 mg, 2.235 mmol) in DMF (9 mL) was stirred at rt as N-methylmorpholine (0.8 mL, 7.276 mmol) was added. After 1.5 h at rt, the solution was diluted with water and the product was extracted with ethyl acetate (×2). The organic fractions were washed with water (×1), combined, dried (Na₂SO₄), and concentrated. The residue was partially purified by combiflash using hexanes and ethyl acetate. The impure product was further purified by preparative HPLC to obtain compound 76 (124 mg, 19%). MS [M+H]⁺=573.7

### Step 3

Compound 2 (53 mg, 88%) was prepared from compound 76 (50 mg, 0.086 mmol) in a manner similar to that described in the synthesis of compound 203. ¹H-NMR (400 MHz, CD₃OD) δ 8.71 (br t, J=6.4 Hz, 1H), 8.44 (s, 1H), 8.19 (d, J=2.4 Hz, 1H), 8.09 (d, J=2.4 Hz, 1H), 7.22 (t, J=72.4 Hz, 1H), 3.90 (dd, J=14.4 and 7.2 Hz, 1H), 3.80 (dd, J=14.4 and 4.0 Hz, 1H), 3.74 (m, 1H), 3.57 (dm, J=−13.2 Hz, 1H), 3.20 (td, J=13.4 and 3.2 Hz, 1H), 2.51 (m, 1H), 2.09-2.41 (m, 3H); ¹⁹F NMR (376.1 MHz, CD₃OD) δ −61.88 (s, 3F), −77.96 (s, 6F), −85.99 (d, J=72.4 Hz, 2F), −95.45 (d, J=244.8 Hz, 1F), −103.42 (dtt, J=244.8, 32.3 and 10.7 Hz, 1F); MS [M+H]⁺=474.2

### Preparation of Compound 204

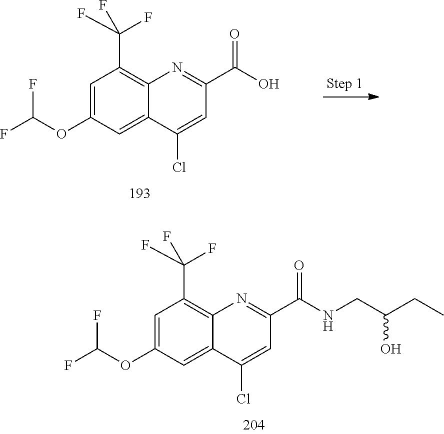

Compound 204 (343 mg, 94%) was prepared from compound 193 (303 mg, 0.888 mmol) in a manner similar to that described previously. ¹H-NMR (400 MHz, CDCl₃) δ 8.50 (s, 1H), 8.44 (br, 1H), 8.14 (d, J=2.0 Hz, 1H), 7.97 (d, J=2.0 Hz, 1H), 6.75 (t, J=72.0 Hz, 1H), 3.78 (m, 1H), 3.74 (ddd, J=13.6, 6.4 and 3.2 Hz, 1H), 3.46 (ddd, J=13.6, 7.6 and 5.6 Hz, 1H), 2.16 (br, 1H), 1.60 (m, 2H), 1.03 (t, J=7.6 Hz, 3H); ¹⁹F NMR (376.1 MHz, CDCl₃) δ −60.78 (s, 3F), −82.96 (d, J=72.0 Hz, 2F); MS [M+H]⁺=413.0

### Preparation of Compound 205

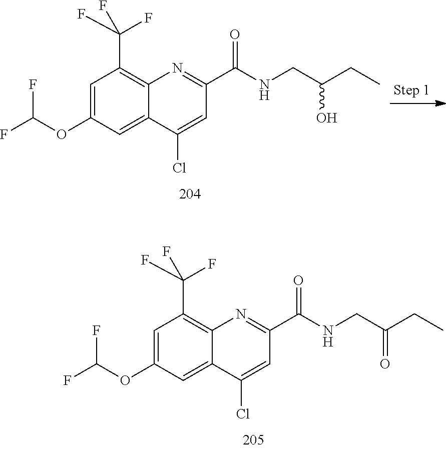

A solution of compound 204 (301 mg, 0.728 mmol) in dichloromethane (15 mL) was stirred at rt as Dess-Martin periodinane (339 mg, 0.799 mmol) was added. After 30 min at rt, additional Dess-Martin periodinane (169 mg, 0.399 mmol) was added. After 20 min, the mixture was diluted with dichloromethane and water containing Na₂S₂O₃ and NaHCO₃. The separated aqueous fraction was extracted with dichloromethane (×1). The organic fractions were washed with brine (×1), combined, dried (Na₂SO₄), and concentrated. The residue was purified by combiflash using hexanes and dichloromethane to obtain compound 205 (204 mg, 68%). ¹H-NMR (400 MHz, CDCl₃) δ 8.75 (br, 1H), 8.49 (s, 1H), 8.15 (d, J=1.6 Hz, 1H), 8.00 (d, J=1.6 Hz, 1H), 6.75 (t, J=72.0 Hz, 1H), 4.40 (d, J=5.2 Hz, 2H), 2.58 (q, J=7.4 Hz, 2H), 1.19 (t, J=7.4 Hz, 3H); ¹⁹F NMR (376.1 MHz, CDCl₃) δ −60.78 (s, 3F), −82.94 (d, J=72.0 Hz, 2F); MS [M+H]⁺=411.2

### Preparation of Compound 206

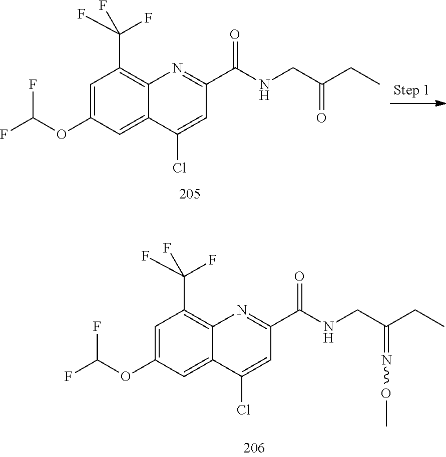

A suspension of compound 205 (196 mg, 0.478 mmol), methoxylamine hydrochloride (205 mg, 2.455 mmol), and sodium acetate (198 mg, 2.414 mmol) in water (2.5 mL) and ethanol (10 mL) was stirred at it for 16 h. The mixture was diluted with water and the product was extracted with ethyl acetate (×2). The organic fractions were washed with water (×1), combined, dried (Na₂SO₄), and concentrated. The residue was purified by combiflash using hexanes and ethyl acetate to obtain compound 206 (205 mg, 98%) as ˜7:3 mixture of two oxime isomers. ¹H-NMR (400 MHz, CDCl₃) δ 8.91 (br, 0.7H), 8.72 (br, 0.3H), 8.52 (s, 0.7H), 8.51 (s, 0.3H), 8.16 (d, J=2.0 Hz, 1H), 7.99 (d, J=2.0 Hz, 1H), 6.75 (t, J=72.0 Hz, 1H), 4.34 (d, J=6.4 Hz, 0.6H), 4.34 (d, J=6.4 Hz, 0.6H), 4.24 (d, J=4.4 Hz, 1.4H), 3.96 (s, 0.9H), 3.96 (s, 2.1H), 2.40 (q, J=7.4 Hz, 1.4H), 2.33 (q, J=7.3 Hz, 0.6H), 1.15 (t, J=7.4 Hz, 0.6H), 1.13 (t, J=7.4 Hz, 1.4H); ¹⁹F NMR (376.1 MHz, CDCl₃) δ −60.66 (s, 2.1F), −60.82 (s, 0.9F), −82.90 (d, J=72.0 Hz, 1.4F), −82.94 (d, J=72.0 Hz, 0.6F); MS [M+H]⁺=440.0

### Preparation of Compound 207

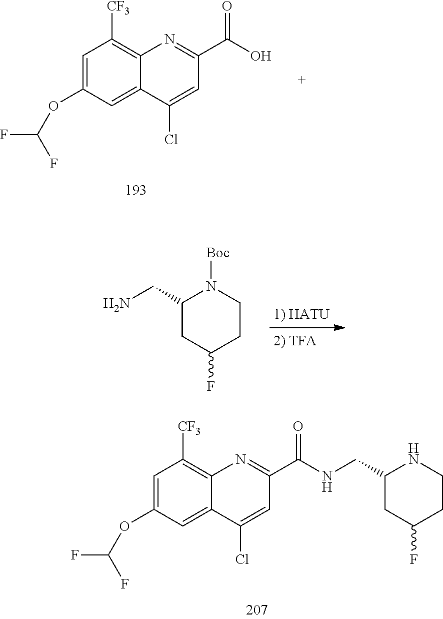

Compound 207 was made using 6-difluoromethoxy-4-methyl-8-trifluoromethyl-quinoline-2-carboxylic acid and 3-aminomethyl-morpholine-4-carboxylic acid tert-butyl ester by HATU coupling according to above procedure, followed by TFA treatment.

Compound 207: (400 MHz, DMSO-d6): 8.44 (bs, 1H), 8.22 (s, 1H), 7.89 (s, 1H), 7.88 (s, 1H), 6.68 (t, 1H), 3.89-3.76 (m, 2H), 3.58-3.34 (m, 4H), 3.39 (m, 1H), 2.96 (m, 2H), 2.76 (s, 3H). ¹⁹F NMR (376.1 MHz) δ −60.59 (s, 3F), 82.30 (d, 2F); MS [M+H]⁺=420.08.

### Example 25

### Preparation of compound 210

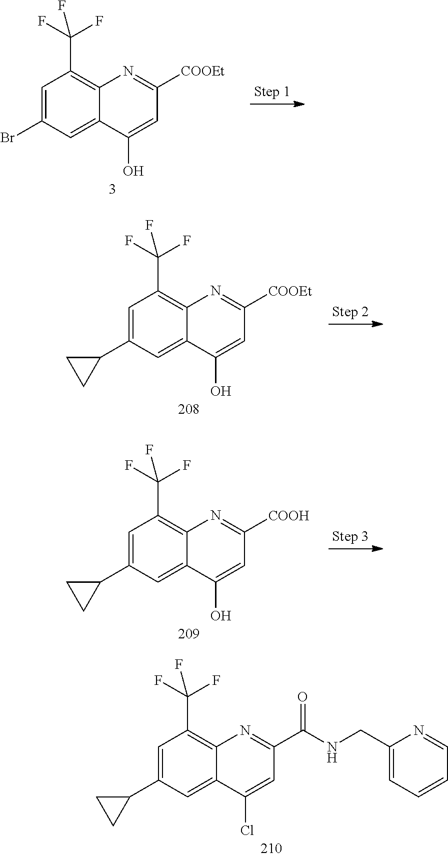

### Step 1

Compound 208 (3.086 g, 34%) was prepared from 3 in a manner described previously. MS [M+H]⁺=326.1

### Step 2

Compound 209 (314 mg, 69%) was prepared from 208 in a manner similar to that described previously. MS [M+H]⁺=298.1

### Step 3

To a solution of compound 209 (314 mg, 1.06 mmol) in thionyl chloride (15 mL) was added DMF (4 drops) at rt and the resulting solution was refluxed for 24 h. The mixture was concentrated and the residue was coevaporated with toluene (×2).

The residue was dissolved in DMF (1.5 mL) with 2-aminomethylpyridine (0.1 mL, 0.978 mmol) and N-methylmorpholine (0.15 mL, 1.364 mmol) at 0° C. After 30 min at 0° C., the mixture was diluted with water and the product was extracted with ethyl acetate (×2). The extracts were washed with water, combined, dried (Na₂SO₄), and concentrated. The residue was purified by combiflash using hexanes and ethyl acetate to obtain compound 210 (136 mg, 69%). ¹H-NMR (400 MHz, CDCl₃) δ 9.12 (br t, J=5.2 Hz, 1H), 8.61 (m, 1H), 8.43 (s, 1H), 8.08 (d, J=2.0 Hz, 1H), 7.83 (d, J=1.6 Hz, 1H), 7.66 (td, J=7.6 and 2.0 Hz, 1H), 7.34 (d, J=8.0 Hz, 1H), 7.20 (dd, J=7.2 and 4.8 Hz, 1H), 4.84 (d, J=5.6 Hz, 2H), 2.17 (m, 1H), 1.16-1.21 (m, 2H), 0.89-0.93 (m, 2H); ¹⁹F NMR (376.1 MHz, CDCl₃) δ −60.43 (s, 3F); MS [M+H]⁺=406.1

### Preparation of Compound 211

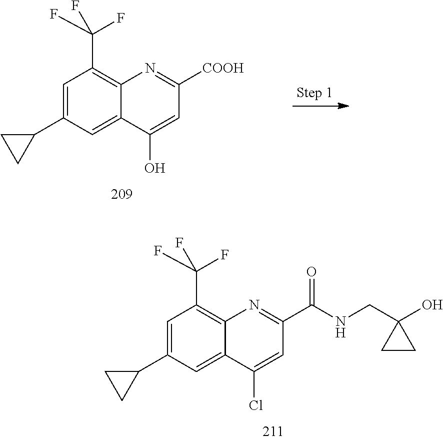

Compound 211 (92 mg, 42%) was prepared from 209 in a manner similar to that described previously. ¹H-NMR (400 MHz, CDCl₃) δ 8.52 (br, 1H), 8.44 (s, 1H), 8.13 (d, J=1.6 Hz, 1H), 7.86 (d, J=1.6 Hz, 1H), 3.69 (d, J=6.0 Hz, 2H), 2.21 (m, 1H), 1.20-1.26 (m, 3H), 0.92-0.96 (m, 2H), 0.88-0.91 (m, 2H), 0.70-0.73 (m, 2H); ¹⁹F NMR (376.1 MHz, CDCl₃) δ −60.44 (s, 3F); MS [M+H]⁺=385.0

### Preparation of Compound 211

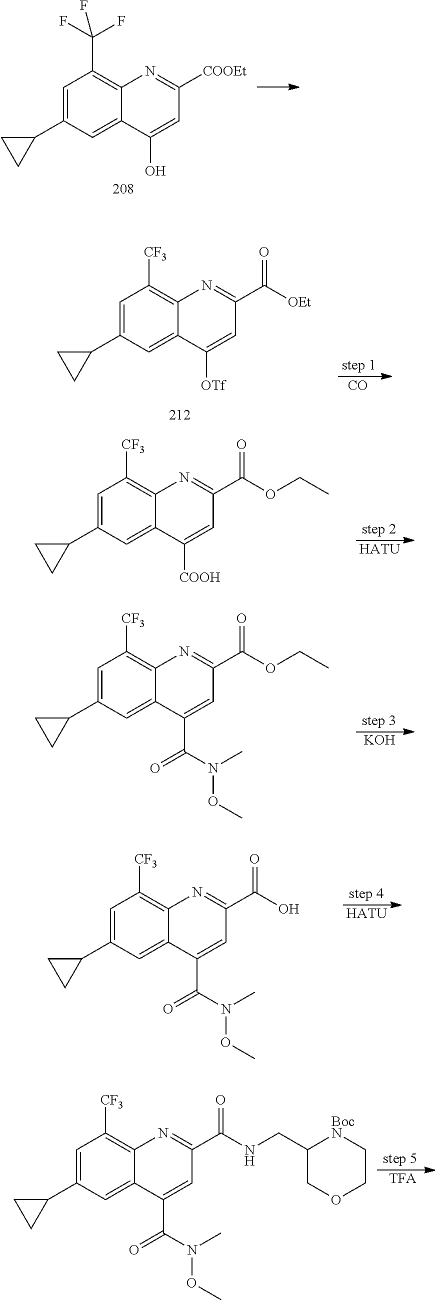

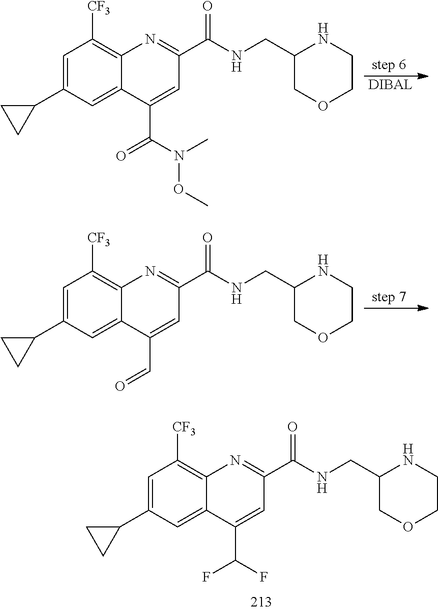

### Step 1

Compounds 6-Cyclopropyl-4-trifluoromethanesulfonyloxy-8-trifluoromethyl-quinoline-2-carboxylic acid ethyl ester 212 (prepared from 208) (0.5 g, 1.1 mmol), DPPP (0.14 g, 0.33 mmol), Pd(OAc)₂ (0.05 g, 0.22 mmol) and triethylamine (0.16 g, 1.54 mmol) were mixed in co solvent DMF/H₂O (10 ml/1 ml). The mixture was flushed with CO (5×) and stirred with a CO balloon on top of it at 60° C. for three hours. The reaction mixture was diluted with EtOAc (15 ml) and was washed with LiCl (5%), HCl (1 N) and brine. Organic phase was dried with sodium sulfate. After removal of the solvent in vacuo, crude compound (17) was obtained and no further purification was performed.

### Step 2, 3, 4, 5, 6 and 7

The procedures were described previously.

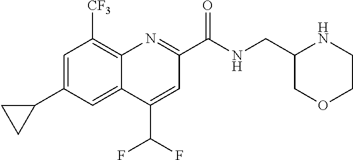

Compound 213: ¹H-NMR (400 MHz, CDCl3) δ 8.46 (t, 1H), 8.35 (s, 1H), 7.97 (s, 1H), 7.81 (s, 1H), 7.09 (s, 1H), 4.12-3.83 (m, 5H), 3.72 (m, 2H), 3.58 (m, 2H), 3.25 (m, 1H), 2.17 (m, 1H), 1.20 (m, 2H), 0.918 (m, 2H); ¹⁹F NMR (376.1 MHz) δ −60.49 (s), −76.47 (s); MS [M−H]⁺=430.17.

The following compounds were prepared from compound 212 using appropriate reagents.

### Compound 214

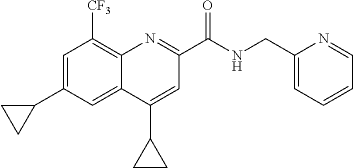

¹H-NMR (400 MHz, CD3OD) δ 8.52 (dd, 1H), 8.38 (d, 1H), 7.89 (s, 1H), 7.87 (s, 1H), 7.80 (t, 1H), 7.31 (t, 1H), 4.77 (d, 2H), 2.62 (m, 1H), 2.28 (m, 1H), 1.27 (m, 2H), 1.18 (m, 2H), 0.94 (m, 4H); ¹⁹F NMR (376.1 MHz) δ −61.72 (s); MS [M−H]⁺=412.17.

### Compound 215

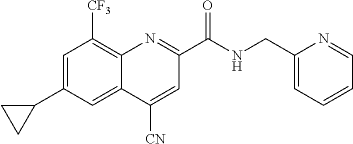

¹H-NMR (400 MHz, DMSO) δ 9.11 (t, 1H), 8.67 (d, 1H), 8.64 (s, 1H), 8.14 (t, 1H), 8.03 (m, 2H), 7.37 (d, 1H), 7.59 (t, 1H), 4.82 (d, 2H), 2.39 (m, 1H), 1.16 (m, 1H), 0.98 (m, 2H); ¹⁹F NMR (376.1 MHz) δ −58.77 (s), −75.52 (s); MS [M−H]⁺=397.15.

### Compound 216

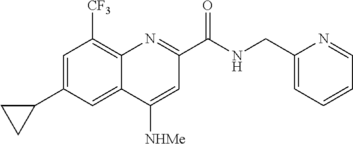

¹H-NMR (400 MHz, CDCl3) δ 9.17 (t, 1H), 8.60 (d, 1H), 7.65 (m, 3H), 7.36 (m, 2H), 7.18 (t, 1H), 4.83 (d, 2H), 3.08 (d, 3H), 2.08 (m, 1H), 1.24 (m, 2H), 0.80 (m, 2H); ¹⁹F NMR (376.1 MHz) δ −60.61 (s); MS [M−H]⁺=401.3.

### Compound 217

Compound 217 was prepared from compound 208.

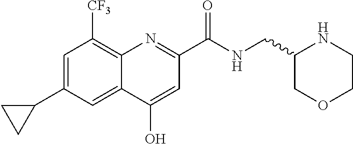

¹H-NMR (400 MHz, CH₃OH-d₄) δ 8.08 (s, 1H), 7.82 (s, 1H), 7.41 (s, 1H), 4.10-3.20 (m, 9H), 2.20 (m, 1H), 1.24 (m, 2H), 0.96 (m, 2H); ¹⁹F NMR (400 MHz, CH₃OH-d₄) δ −61.58 (s); MS [M+H]⁺=396.

### Compound 218

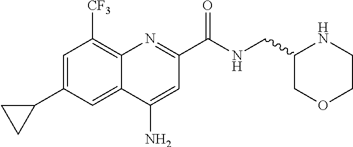

¹H-NMR (400 MHz, CH₃OH-d₄) δ 8.18 (s, 1H), 8.04 (s, 1H), 7.86 (s, 1H), 4.15-3.25 (m, 9H), 2.30 (m, 1H), 1.24 (m, 2H), 0.96 (m, 2H); ¹⁹F NMR (400 MHz, CH₃OH-d₄) δ −61.58 (s); MS [M+H]⁺=395.

### Preparation of Compounds 220 and 221

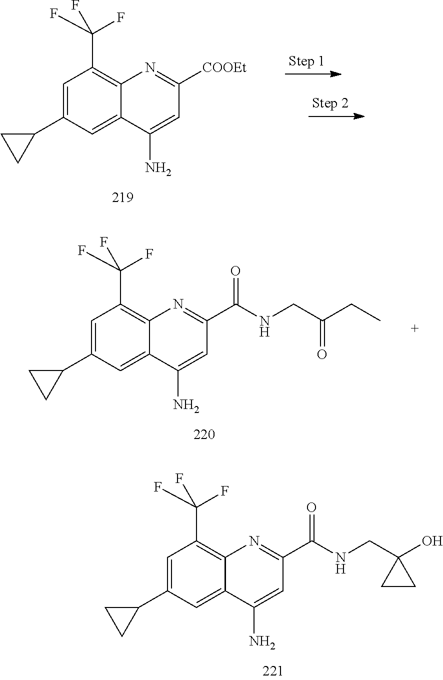

### Step 1 and 2

Compound 220 and 221 were prepared from compound 219 (which was prepared from compound 209) in a manner similar to that described previously using a mixture (˜4:1 ratio) of the corresponding amines. Two products were purified by combiflash using hexanes and ethyl acetate to obtain compound 220 (21 mg, 77%) and impure 221.

Compound 220: ¹H-NMR (400 MHz, CDCl₃) δ 8.89 (br, 1H), 7.72 (s, 1H), 7.64 (s, 1H), 7.48 (s, 1H), 4.36 (d, J=5.2 Hz, 2H), 2.57 (q, J=7.4 Hz, 2H), 2.11 (m, 1H), 1.17 (t, J=7.4 Hz, 3H), 1.10-1.13 (m, 2H), 0.83-0.87 (m, 2H); ¹⁹F NMR (376.1 MHz, CDCl₃) δ −60.72 (s, 3H); MS [M+H]⁺=366.1

The impure 221 was further purified by preparative HPLC and the collected pure fraction was concentrated, dissolved in ethyl acetate and washed with saturated NaHCO₃ solution before drying (Na₂SO₄) and concentration to obtain compound 52 (4.3 mg, 16%).

Compound 221: ¹H-NMR (400 MHz, CD₃OD) δ 8.03 (br, 1H), 7.82 (br, 1H), 7.37 (s, 1H), 3.59 (s, 2H), 2.16 (m, 1H), 1.09-1.14 (m, 2H), 0.87-0.91 (m, 2H), 0.73-0.78 (m, 2H), 0.67-0.71 (m, 2H); ¹⁹F NMR (376.1 MHz, CDCl₃) δ −62.03 (s, 3F); MS [M+H]⁺=366.1

### Preparation of Compound 222

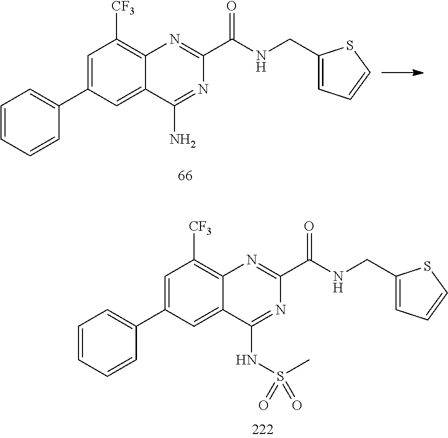

Amine 66 (30 mg, 0.070 mmol) dissolved in DCM (2 ml) was added DIPEA (24 μl, 0.140 mmol) and MsCl (65 μl, 0.084 mmol) at RT under N₂. After stirred for 3 hrs, the reaction was completed and concentrated. The crude product was purified by HPLC to give 12 mg (34%) of compound 222.

¹H-NMR (400 MHz, DMSO-d6) δ 9.72 (m, 1H), 8.61 (s, 1H), 8.26 (s, 1H), 7.77 (d, 2H), 7.52 (m, 2H), 7.40 (m, 2H), 7.04 (m, 1H), 6.95 (m, 1H), 4.69 (d, 2H), 3.06 (s, 3H); ¹⁹F NMR (376.1 MHz) δ −59.57 (s); MS [M+H]⁺=507.0.

### Example 26

### Preparation of Compound 226

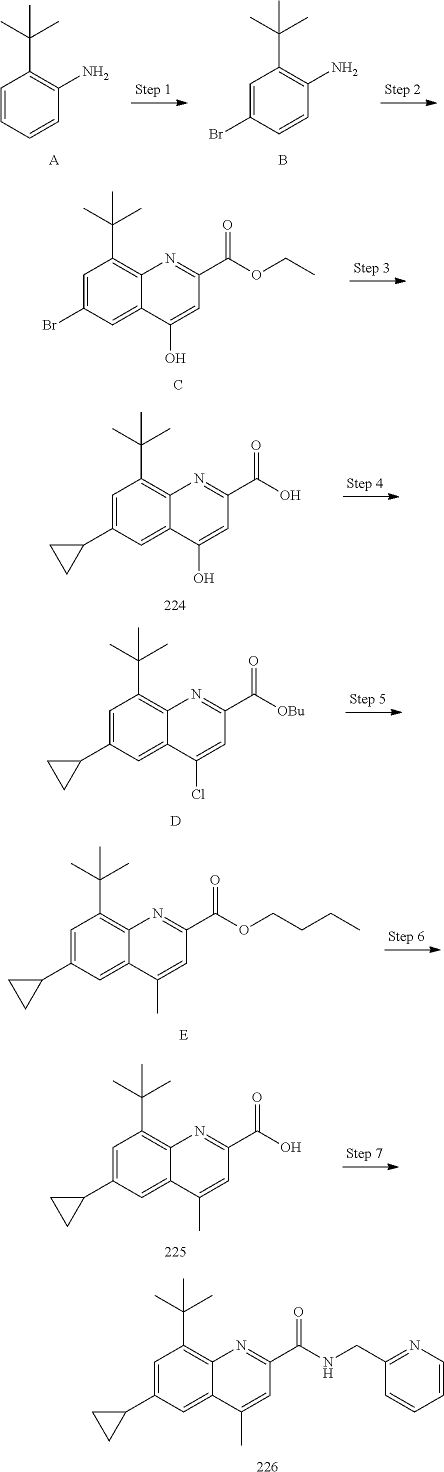

### Step 1

A was brominated by NBS to afford B

### Step 2

4-Bromo-2-trifluoromethyl-phenylamine (72.9 g, 0.32 mol) and But-2-ynedioic acid diethyl ester (59.9 g, 0.353 mol) were dissolved in MeOH (120 ml) in a 500 ml round bottom flask and refluxed for 2 h. The volatiles were removed in vacuo, and the residue was placed in a pre-heated reaction block (220° C.) and fitted with a vacuum adapter and a thermocouple (J-KEM). The reaction was heated under house vacuum (˜80 torr) until the internal temperature reached 187° C. (˜45 min). Analysis by LCMS indicates the reaction was complete. The reaction was allowed to cool in air with vigorous stirring using an overhead stirrer. Once the reaction had cooled to 120° C., heptane was added portionwise, providing a thick slurry of crystalline product. The slurry was stirred at reflux for one hour, then cooled to it and stirred overnight. Filtration provided the desired compound C (81.1 g, 72% Yield) and a tan powder. MS [M+H]⁺=352.23 (100%), 354.0 (90%).

### Step 3

A 2-L 3-neck Morton flask was charged with Palladium acetate (1.4 g, 6.36 mmol) and [1,1′-biphenyl]-2-yldicyclohexyl-phosphine (4.45 g, 12.7 mmol). The flask was evacuated and backfilled with N2 (3×) and 500 mL dioxane was added via cannula. After stirring for 15 min under N2, potassium trifluorocylopropyl borate (34.1 g, 239 mmol), potassium phosphate (135 g, 636 mmol) and compound C (55.7 g, 159 mmol) were added. 50 ml degassed water was added, and the reaction stirred under vacuum for 1 min, then back-filled with N2 (3×). The reaction was then heated to 100° C. overnight. The reaction was diluted with 750 ml water and cooled to rt. The mixture was acidified with conc HCl and the solid precipitate filtered and dried in a vacuum oven overnight to provide

Compound 224 (41.1 g, 91%) as a black solid.

MS [M+H]⁺=286.18 (100%), 287.17 (98%).

### Step 4

A solution of compound 224 (10.1 g, mmol) in 150 mL thionyl chloride was heated at 65° C. for 2 h. The volatiles were removed in vacuo and the residue was then taken up in toluene and concentrated again. The residue was dissolved in 100 mL DCM and treated with 16 mL 1-butanol and 30 mL pyridine. After stirring for 10 min, the reaction was diluted with chloroform (250 mL) and 1 N HCL. The organic layer was separated, washed with brine and dried with sodium sulfate. After removal of the volatiles in vacuo, the residue was purified by column chromatography to provide Compound D as a lightly colored oil (7.9 g, mmol) MS[M+H]=318.35 (100%), 320.33 (30%).

### Step 5

A well degassed solution of compound D (3.31 g, 10 mmol) and palladium 1,2-Bis(diphenylphosphino)ethane in (56 mg, 0.1 mmol) in 50 mL dioxane was treated with dimethyl zinc (15 mL, 30 mmol, 2M in toluene) and the mixture heated to 100° C. for 5 h. The reaction was then cooled to −10° C. and quenched with 2N HCl and EtOAc. Aqueous work-up provided Compound E as a yellow oil. MS [M+H]⁺=312.10 (100%), 313.13 (20%)

### Step 6

Compound E was taken up in 150 mL THF and treated with LiOH (40 mL, 40 mmol, 1 N in water) and heated to 60° C. overnight. Acidic aqueous work-up (2.5 N HCl. MS [M+H]⁺=284.13.

### Step 7

Compound 225 (290 mg, 0.63 mmol) and 2-methylamino pyridine (96 uL, 0.94 mmol) were dissolved in DMF (1.5 ml), and treated with NMM (203 uL, 1.88 mmol) and BOP (416 mg, 0.94 mmol). After 5 min stirring, the reaction was purified by prep-HPLC to afford compound H as a white solid light brown solid 226 (298 mg, 96% yield). ¹H-NMR (400 MHz, DMSO-d6) δ 9.20 (t, J=4 Hz, 1H), 8.57 (m, 1H), 7.97 (s, 1H), 7.78 (td, J=12, 4 Hz, 1H), 7.62 (d, J=4 Hz, 1H), 7.42 (m, 2H), 7.40 (m, 1H), 7.31 (m, 1H), 4.72 (d, J=4 Hz, 2H), 2.71 (s, 3H), 2.16 (m, 1H), 1.66 (s, 9H), 1.07 (m, 2H), 0.87 (m, 2H); MS [M+H]⁺=402.23.

### Preparation of Compound 227

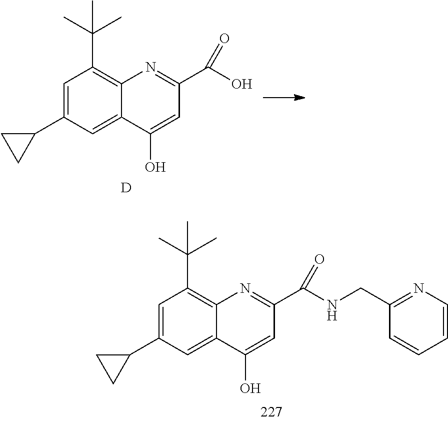

Compound 227 was prepared using the same procedure described previously. ¹H-NMR (400 MHz, DMSO-d6) δ 11.53 (bs, 1H), 9.17 (s, 1H), 8.57 (bs, 1H), 7.81 (t, J=8 Hz, 1H), 7.67 (s, 1H), 7.49 (s, 1H), 7.41 (m, 2H), 7.32 (m, 1H), 4.70 (d, J=4 Hz, 1H), 2.11 (m, 1H), 1.69 (s, 9H), 1.01 (m, 2H), 0.77 (m, 2H); MS [M+H]⁺=375.

### Preparation of Compound 228

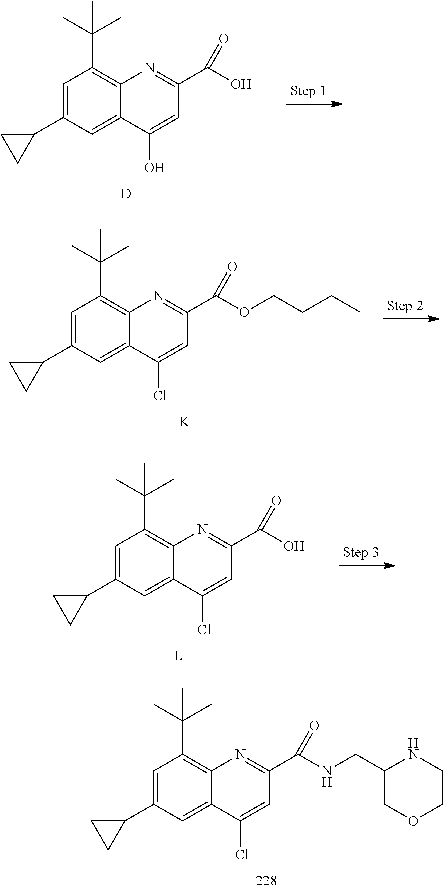

The procedure used to prepare compound 228 was same as step 7 in example compound 226. ¹H-NMR (400 MHz, DMSO-d6) δ 9.23 (bs, 1H), 9.10 (bs, 1H), 8.20 (m, 1H), 7.93 (s, 1H), 7.61 (s, 1H), 7.41 (s, 1H), 3.96 (d, J=12 Hz, 1H), 3.85 (d, J=12 Hz, 1H), 3.75-3.4 (m, 5H), 3.22 (m, 1H), 3.05 (m, 1H), 2.15 (m, 1H), 1.62 (s, 9H), 1.04 (m, 2H), 0.86 (m, 2H); MS [M+H]⁺=401.2.

### Preparation of Compound 229

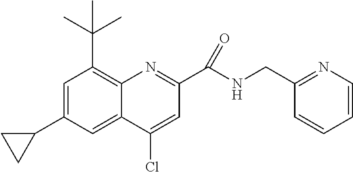

¹H-NMR (400 MHz, DMSO-d6) δ 9.22 (bs, 1H), 8.57 (d, J=4 Hz, 1H), 8.18 (s, 1H), 7.79 (t, J=8 Hz, 1H), 7.77 (s, 1H), 7.54 (s, 1H), 7.73 (d, J=8 Hz, 1H), 7.32 (m, 1H), 4.73 (d, J=8 Hz, 2H), 2.23 (m, 1H), 1.67 (s, 9H), 1.09 (m, 2H), 0.88 (m, 2H); MS [M+H]⁺=394.4.

### Preparation of Compound 230

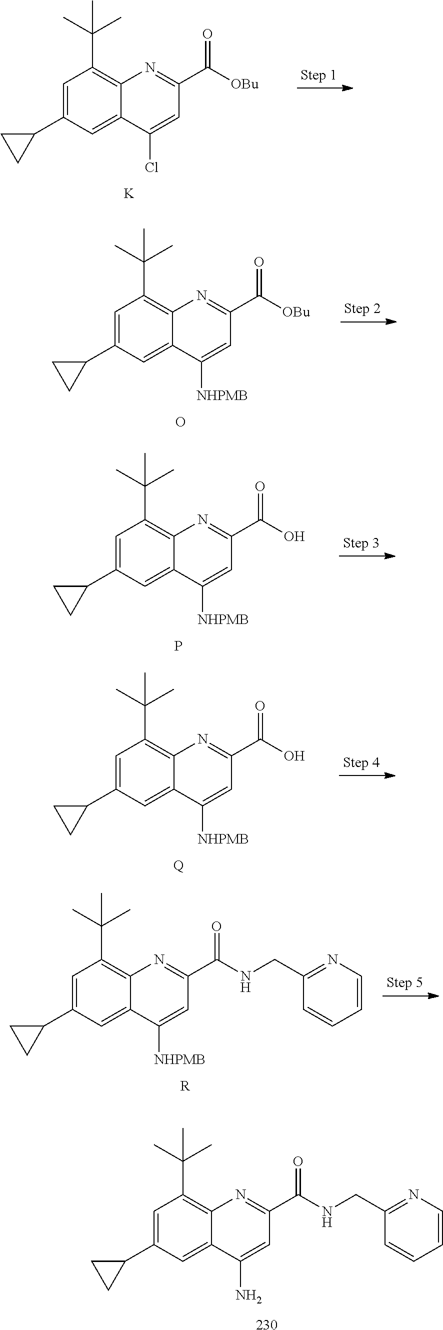

### Step 1

A 1-L 3 neck rbf was charged with Pd₂(dba)₃ (672 mg, 0.175 mmol), DavePhos® (1.18 g, 3.01 mmol) and cesium carbonate (29.3 g, 90.3 mmol). The reaction flask was evacuated and back-filled with N2 (3×) and the solids taken up in 250 mL dioxane. After 5 min stirring, a solution of compound K (10.8 g, 30.1 mmol) in degassed dioxane was added, followed by p-methoxybenzylamine (5.8 mL, 45 mmol). The reaction mixture was heated at 100° C. overnight. Aqueous work-up (EtOAc, water) and silica gel chromatography provided compound O (11.1 g, 83% yield) as a tan solid. MS [M+H]⁺=461.36.

### Step 2

Compound O was hydrolyzed using the same procedure in Example 1, step 6 to provide Compound P.

MS [M+H]⁺=405.31.

### Step 3 (180-1)

Compound P was reacted using the same procedure as Example I, step 7. MS[M+H]=405.31

### Step 4

Compound R was prepared using the same procedure as Example I, step 7 to provide Compound R. MS[M+H]=495.42

### Step 5

Compound P (293 mg, 0.60 mmol) was taken up in 15 mL TFA at rt and treated with p-TsOH-H₂O (190.22 mg, 2.1 mmol). After 5 min the reaction was diluted with EtOAc and 250 mL 10% K₂CO₃. The organic layer was dried (sodium sulfate) and concentrated to provide a solid. This material was purified by Prep. HPLC to yield compound 230 (212 mg) as a white solid. 400 MHz ¹H NMR (DMSO): 7.69 (s, 1H), 7.65 (bs, 2H), 7.49 (s, 1H), 7.14 (s, 1H), 3.67 (m, 2H), 2.05 (m, 1H), 1.56 (s, 9H), 1.01 (m, 2H), 0.84 (m, 1H). MS[M+H]=375.26.

### Preparation of Compound 231

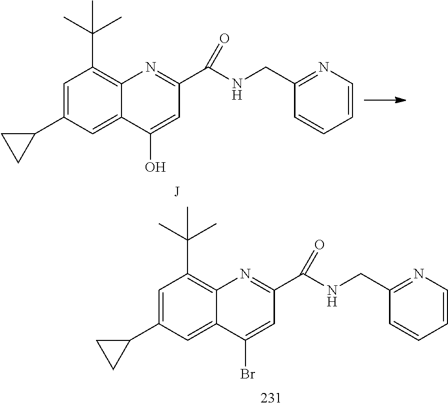

Compound J (155 mg, 0.41 mmol) was taken up in 15 mL DCE and treated with 1 g phosphorous oxybromide The mixture was heated at 75° C. for 2 h, then quenched with water. Aqueous work up (EtOAc, 10% K₂CO₃) and trituration with ether provided compound 231 (145 mg, 0.38 mmol)as a brown solid. 400 MHz ¹H NMR (DMSO): ¹H-NMR (400 MHz, DMSO-d6) δ 9.20 (t, J=4 Hz, 1H), 8.57 (m, 1H), 7.97 (s, 1H), 7.78 (td, J=12, 4 Hz, 1H), 7.62 (d, J=4 Hz, 1H), 7.42 (m, 2H), 7.40 (m, 1H), 7.31 (m, 1H), 4.72 (d, J=4 Hz, 2H), 2.16 (m, 1H), 1.66 (s, 9H), 1.07 (m, 2H), 0.87 (m, 2H); MS[M+H]=438.44 (100%), 440.18 (98%).

### Example 27

### Preparation of Compounds 232-235

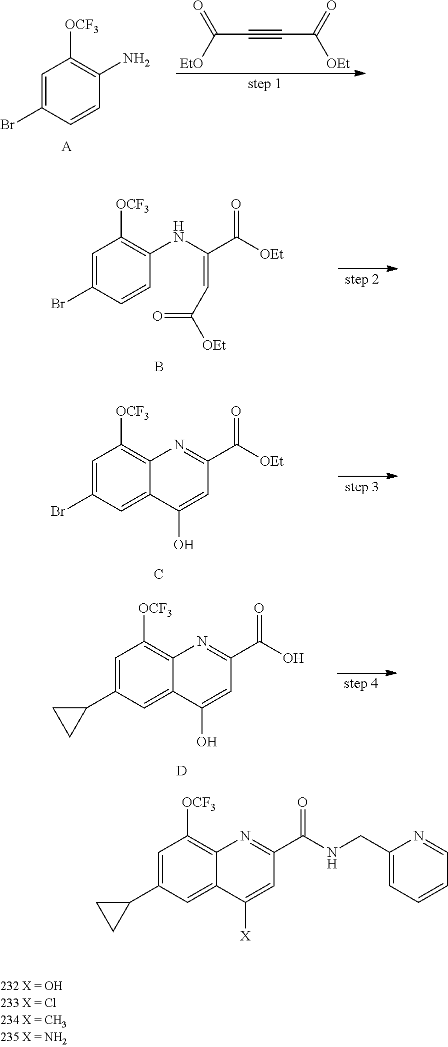

### Step 1

4-Bromo-2-trifluoromethoxy-phenylamine (13 g, 0.05 mol) and But-2-ynedioic acid diethyl ester (10.3 g, 0.12 mol) were dissolved in EtOH (120 ml) in a 500 ml round bottom flask and refluxed. The reaction was monitored by LC-MS. 3 h later; 0.3 eq. but-2-ynedioic acid diethyl ester was added. At t=5 h, the reaction mixture was concentrated down to remove the solvent under vacuum, a thick oil was obtained and was used without purification for the next step. MS [M+H]⁺=426.

### Step 2

A heat gun was set to produce a temperature of approximately 400° C. The crude material B obtained from the previous step was dissolved in Ph₂O (100 ml) in a 500 ml round bottom flask attached to a condenser, and the reaction mixture was placed over the heat gun. After 5 min, the solvent temperature had reached 260° C. as evidenced by rapid boiling, and the solution color changed from yellow to green then to brown. After 15 minutes Ph₂O reflux, the heat gun was removed. At this time, reaction product precipitated out as a light tan solid upon cooling to room temperature. This solid was filtered and washed with hexane, mother liquor was concentrated and more solid crashed out, the above procedure was repeated to recover additional product. 9 g of product C was obtained. MS [M+H]⁺=380.

### Step 3

To a mixture of compound C (0.4 g, 1.1 mmol), cyclopropyl boronic acid (0.64 g, 2.2 mmol), Pd(dppf)Cl₂ (0.13 g, 0.11 mmol) in a conical reaction vessel was added dioxane (5 ml) and K₃PO₄ (1M) (3.3 ml). The reaction mixture was placed in microwave reactor at 120° C. for 30 minutes. After cooling, Pd catalyst and by-products were filtered off. When the mixture was acidified with HCl (2N) to pH=4, solid product 4 was precipitated out. The filter cake was washed with water followed by hexane, and dried under high vacuum to afford light brown color solid. The crude material 504 was taken forward to next step without further purification. MS [M+H]=314; LCMS rt=1.85 min.

### Step 4

### Synthesis of Compound 232

Acid D (0.2 g, 0.5 mmol) and 2-aminomethylpyridine (0.1 g, 1.2 mmol) were dissolved in DMF (15 ml), followed by the addition of EDCl (0.3 g, 1.6 mmol), HOBt (0.2 g, 1.6 mmol), and NMM (0.2 g, 2.5 mmol). The reaction was stirred at rt for overnight, and monitored by LC-MS. Reaction mixture was purified by prep-HPLC to afford light brown solid 232 (0.1 g, 0.2 mmol). ¹H-NMR (400 MHz, DMSO-d6) δ 9.22 (m, 2H), 8.31 (m, 1H), 8.01 (m, 1H), 7.75 (s, 1H), 7.74 (m, 2H), 7.55 (m, 2H), 7.35 (m, 2H), 7.29 (m, 2H), 4.58 (d, 2H), 2.19 (m, 1H), 1.06 (m, 2H), 0.83 (m, 2H). ¹⁹F NMR (376.1 MHz) δ 56.79 (s), MS [M+H]⁺=404.

### Synthesis of 233

Acid D (0.2 g, 0.6 mmol) was dissolved into 3 mL dioxane and 3 mL POCl₃, followed by catalytic DMF. The reaction was heated to 60° C. for 1 h, at which time volatiles were removed and 2-aminomethylpyridine (0.1 g, 1.2 mmol) were dissolved in dioxane and added to the resulting residue. The reaction was stirred at rt for 15 minutes, and then injected directly onto HPLC. Reaction mixture was purified by prep-HPLC to afford light yellow solid 233 (0.02 g). ¹H-NMR (400 MHz, CDCl₃) δ 9.20 (bs, 1H), 8.60 (m, 1H), 8.42 (s, 1H), 7.89 (s, 1H), 7.72 (m, 1H), 7.39 (s, 1H), 4.86 (d, 2H), 2.14 (m, 1H), 1.19 (m, 2H), 0.89 (m, 2H). MS [M+H]⁺=422.

### Synthesis of 234

A sample of 233, 50 mg, was treated to standard Suzuki coupling conditions using methylboronic acid, as described elsewhere in this document. The resulting product was purified by HPLC to give 20 mg 234 as product. ¹H-NMR (400 MHz, DMSO-d6) δ 9.26 (m, 2H), 8.52 (m, 1H), 8.08 (s, 1H), 7.80 (s, 1H), 7.76 (m, 1H), 7.58 (s, 1H), 7.38 (m, 1H), 7.30 (m, 1H), 4.68 (d, 2H), 2.76 (s, 3H), 2.24 (m, 1H), 1.09 (m, 2H), 0.92 (m, 2H). MS [M+H]⁺=402.

### Synthesis of 235

A sample of heteroaryl chloride 233, 50 mg, was treated to standard nucleophilic amine displacement conditions using DMB-amine as reaction solvent. Final treatment with neat TFA at 50° C., followed by HPLC purification gave 15 mg 235 as product. (400 MHz, CD₃CN) δ 9.13 (bs, 1H), 8.65 (m, 1H), 8.22 (m, 1H), 7.77 (s, 1H), 7.67 (m, 1H), 7.63 (s, 1H), 7.51 (s, 1H), 7.43 (s, 1H), 4.90 (bs, 2H), 2.13 (m, 1H), 1.13 (m, 2H), 0.89 (m, 2H). MS [M+H]⁺=403.

### Example 28

### Preparation of Compounds 236-239

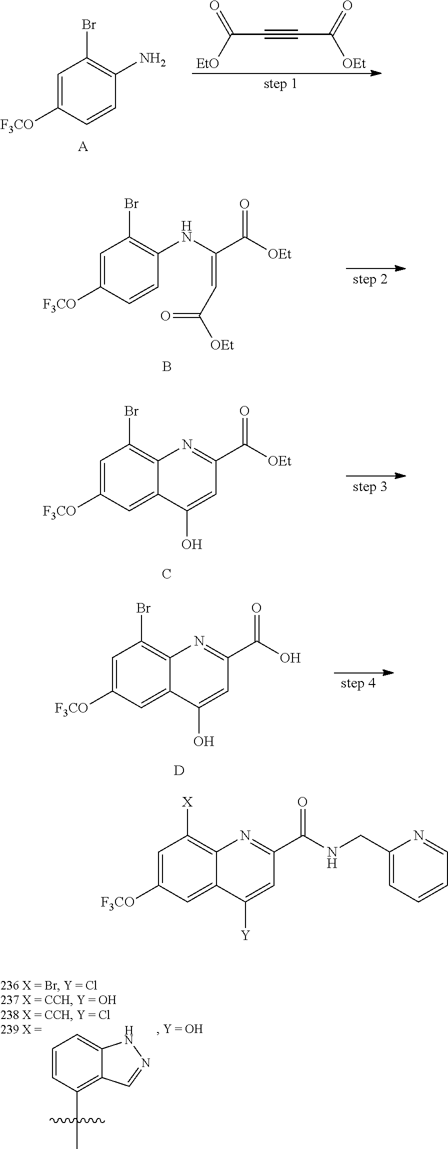

### Step 1

The procedure used was the same as step 1 in Example 27, in this case beginning with 10 g 2-bromo-4-trifluoromethoxy phenylamine to afford compound B. MS [M+H]⁺=426.

### Step 2

The procedure utilized was same as step 2 in Example 27 to afford 10.5 g compound C upon precipitation. MS [M+H]⁺=380.

### Step 3

Standard procedure for the hydrolysis of C with LiOH in THF/water was utilized to furnish acid B. MS [M+H]⁺=352.

### Synthesis of Heteroaryl Bromide 236

Acid D (0.2 g, 0.6 mmol) and 2-aminomethylpyridine (0.1 g, 1.2 mmol) were dissolved in DMF (15 ml), followed by the addition of EDCl (0.3 g, 01.6 mmol), HOBt (0.2 g, 1.6 mmol), and NMM (0.2 g, 2.5 mmol). The reaction was stirred at rt for overnight, and monitored by LC-MS. Reaction mixture was purified by prep-HPLC to afford light brown solid 236 (0.1 g, 0.02 mmol). ¹H-NMR (400 MHz, DMSO-d6) δ 9.35 (m, 1H), 8.55 (m, 1H), 8.47 (s, 1H), 8.39 (s, 1H), 7.18 (s, 1H), 7.74 (m, 1H), 7.38 (m, 1H), 7.29, 4.72 (d, 2H). MS [M+H]⁺=462.

### Synthesis of Acetylene 237

In a procedure utilizing TMS acetylene, CuI, Pd(dppf)Cl₂, and TEA at 110° C., bromide C was converted to the corresponding acetylene. After standard hydrolysis of the ester with aq. LiOH, during which the silyl group was also seen to cleave, the resulting acid (0.2 g, 0.6 mmol) and 2-aminomethylpyridine (0.1 g, 0.9 mmol) were dissolved in DMF (15 ml), followed by the addition of EDCl (0.3 g, 01.6 mmol), HOBt (0.2 g, 1.6 mmol), and NMM (0.2 g, 2.5 mmol). The reaction was stirred at it for overnight, and monitored by LC-MS. Reaction mixture was purified by prep-HPLC to afford light brown solid 237 (0.1 g, 0.02 mmol). ¹H-NMR (400 MHz, DMSO-d6) δ 12.26 (bs, 1H), 8.66 (m, 1H), 8.53 (m, 1H), 8.40 (s, 1H), 8.34 (m, 1H), 7.61 (s, 1H), 7.32 (m, 4H), 7.23 (m, 1H), 4.58 (d, 2H). ¹⁹F NMR (376.1 MHz) δ −58.56 (s), 73.98 (s), MS [M+H]⁺=388.

### Synthesis of 238

In a procedure utilizing TMS acetylene, CuI, catalytic Pd(dppf)Cl₂, and TEA as solvent at 110° C., heteroaryl bromide C was converted to the resulting acetylene. After standard hydrolysis of the ester with aq. LiOH, during which the silyl group was also removed, the resulting acid (0.2 g, 0.6 mmol) was dissolved into 3 mL dioxane and 3 mL POCl₃, followed by catalytic DMF. The reaction was heated to 60° C. for 1 h, at which time volatiles were removed and 2-aminomethylpyridine (0.1 g, 0.9 mmol) was dissolved in dioxane and added to the resulting residue. The reaction was stirred at it for 15 minutes, and then injected directly onto HPLC. Reaction mixture was purified by prep-HPLC to afford light yellow solid 238 (0.02 g). ¹H-NMR (400 MHz, DMSO-d6) δ 9.35 (m, 1H), 8.55 (m, 1H), 8.47 (s, 1H), 8.39 (s, 1H), 7.18 (s, 1H), 7.74 (m, 1H), 7.38 (m, 1H), 7.29, 4.72 (d, 2H). MS [M+H]⁺=407.

### Synthesis of 239

Compound C (0.5 g, 0.06 mmol), azaindole 4-boronic acid (0.5 g, 3 mmol), Pd(dppf)Cl₂ (0.13 g, 0.11 mmol) in a microwave tube was added dioxane (5 ml) and K₃PO₄ (1M) (3.3 ml). The reaction mixture was placed in microwave reactor at 120° C. for 30 minutes. LC/MS revealed that the coupling reaction was complete and that ester hydrolysis to the corresponding acid was evident in the major product. Upon filtration to remove insoluble Pd-based by-products followed by concentration of the reaction solvent, the crude material was taken forward to next step without further purification. Standard EDCl coupling of this material gave 1 mg final diaryl product 239 after HPLC purification of a 50 mg sample of the crude residue. ¹H-NMR (400 MHz, DMSO-d6) δ 8.65 (bs, 1H), 8.55 (m, 1H), 8.17 (s, 1H), 7.96 (s, 1H), 7.77 (m, 1H), 7.64 (m, 2H), 7.58-7.34 (cm, 4H), 4.64 (d, 2H). MS [M+H]⁺=480.

### Preparation of Compounds 240 and 241

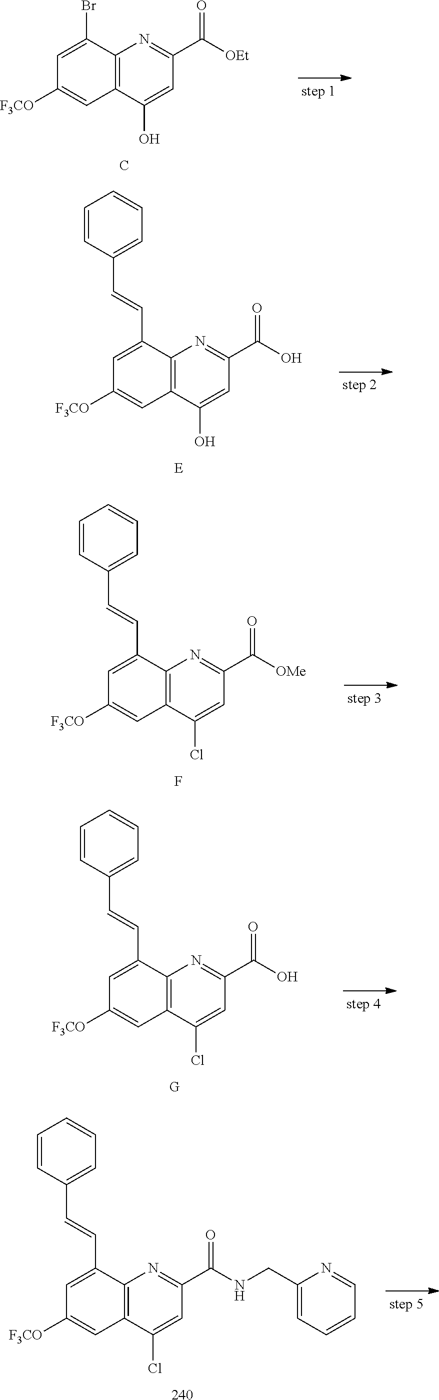

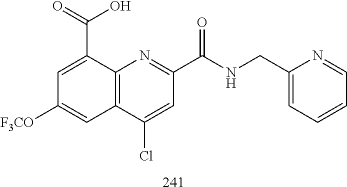

### Step 1

Compound C (0.500 g, 1.32 mmol), styrene boronic acid (0.292 g, 1.98 mmol), Pd(dppf)Cl₂ (0.107 mg, 0.134 mmol) and K₃PO₄ (4 ml, 1M solution) were combined in a 100 ml round bottom flask. The reaction vessel was placed under vacuum and then refilled with Ar three times. 1,4-dioxane (13 ml) was added to the solid mixture. The reaction vessel was heated to 140° C. with stirring. The reaction was monitored by LC-MS, which showed complete conversion of the starting material after 1 hour. After the flask was cooled to room temperature, the mixture was concentrated under vacuum and re-dissolved in EtOAc. The organic solution was washed successively with concentrated NH₄Cl, water and brine and then dried over Na₂SO₄. The solution was concentrated under vacuum and the resulting solid was used in the next step without purification. MS [M+H]⁺=331.12.

### Step 2

Compound E (0.470 g, 0.798 mmol) was dissolved in 10 ml of DCE. Oxalyl chloride (0.8 ml) and DMF (0.050 ml) were added and the mixture was stirred overnight at room temperature. Methanol (10 ml) was added to quench the reaction. The mixture was concentrated under vacuum and re-dissolved in EtOAc. The organic solution was washed successively with concentrated NH₄Cl, water and brine and then dried over Na₂SO₄. The solution was concentrated under vacuum and the resulting solid was used in the next step without purification. MS [M+H]⁺=408.19.

### Step 3

Compound F (300 mg, 0.720 mmol) was dissolved in a 1:1 mixture of methanol and THF (15 ml). Lithium hydroxide (3 ml of 1M solution) was added and the mixture was stirred at room temperature for 1 hour. Complete conversion of the starting material was observed by LC-MS. The organic solvents were removed under vacuum and hydrochloric acid (3 ml of 1M solution) was added. The resulting precipitate was filtered, washed with water and dried under vacuum. MS [M+H]⁺=394.25.

### Step 4

Compound G (0.275 g, 0.700 mmol) was dissolved in 15 ml of DMF in a 50 ml round bottom flask. HATU (1.00 g, 2.63 mmol), N-methylmorpholine (0.665 mg, 6.58 mmol) and 2-aminomethyl-pyridine (0.739 g, 3.95 mmol) were added and the mixture was stirred at room temperature for 1 hour. The solution was purified by HPLC to give compound 240 (150 mg). ¹H-NMR (400 MHz, cdcl₃) δ 9.68 (s, 2H), 8.75-8.54 (m, 6H), 8.33 (s, 6H), 8.08 (d, J=7.5 Hz, 4H), 7.96 (s, 5H), 7.88 (d, J=6.9 Hz, 5H), 7.76 (s, 4H), 7.43 (t, J=7.4 Hz, 5H), 7.31 (dd, J=15.4, 7.7 Hz, 6H), 5.16 (s, 7H). MS [M+H]⁺=484.30.

### Step 5

Compound 240 (0.130 g, 0.267 mmol) was dissolved in DMF (2 ml), and OsO₄ (0.334 ml, 2.5% in tBuOH) was added and stirred for 5 min. Oxone (0.656 g, 1.07 mmol) was added in one portion and the reaction was stirred at room temperature for 3 hours. LC-MS showed completion of the reaction. Sodium sulfite (1.5 mmol) was added and stirred for an additional hour. EtOAc was added to extract the products and 1N HCl was used to dissolve the salts. The organic extract was washed with 1N HCl (3×) and brine, dried over Na₂SO₄, and the solvent was removed under vacuum to obtain the crude product, which was purified by HPLC to give compound 241 (18 mg). ¹H-NMR (400 MHz, DMSO-d6) δ 9.24 (t, J=5.7 Hz, 1H), 8.57 (d, J=4.8 Hz, 1H), 8.40 (s, 1H), 8.27 (d, J=18.3 Hz, 2H), 7.84 (t, J=7.6 Hz, 1H), 7.45 (d, J=8.0 Hz, 1H), 7.41-7.30 (m, 1H), 4.71 (d, J=5.8 Hz, 3H). MS [M+H]⁺=426.21.

### Example 29

### Preparation of Compound 242

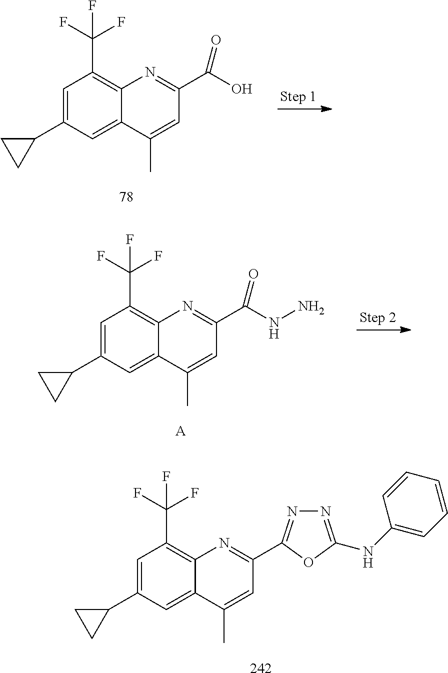

### Step 1

A solution of compound 78 (750 mg, 2.32 mmol), tert-butyl carbazate (462 mg, 3.5 mmol), DIEA (1.9 mL, 11.6 mmol) and Py-BOP (1.5 g, 2.9 mmol) was stirred in DMF for 15 min, then diluted with EtOAc and washed with 1N citric acid (1×), sodium citrate (1×) and LiCl (1×). After removal of the volatiles, the residue was purified by silica gel chromatography. The isolated product was then treated with 50% TFA in DCM for 30 min. After concentration in vacuo, an aqueous work-up (EtOAc, sat. NaHCO3) provided compound CC (371 mg, 46% yield) as a yellow solid. MS [M+H]⁺=310.10.

### Step 2

Compound A (70 mg, 0.19 mmol) and phenyl isothiocyanate (29 uL, 0.21 mmol) were heated in 2 mL DCE at 80 C for 2 h. EDC (215 mg, 0.95 eq) was then added as a solid, and the reaction left to stir overnight. Reaction mixture was concentrated in vacuo and taken up in 3 mL DMF. Purified by prep HPLC to provide compound 242 (31 mg, 40% Yield) as a white powder. 400 MHz ¹H NMR (DMSO): 10.91 (s, 1H), 8.15 (s, 1H), 8.05 (s, 1H), 7.98 (s, 1H), 7.67 (d, J=8 Hz, 2H), 7.35 (ap t., J=8 Hz, 2H), 7.01 (m, 1H) 2.78 (s, 3H), 2.34 (m, 1H), 1.04 (m, 2H), 0.96 (m, 2H) ¹⁹F NMR (376.1 MHz) δ −58.9, −75.0 (s); LCMS [M+H]=411.18.

### Preparation of Compound 243

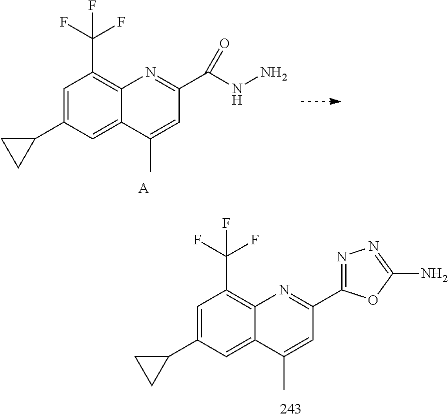

Compound A (343 mg, 1.1 mmol) was taken up in 5 mL dioxane and 5 mL sat. NaHCO3. Cyanogen bromide (117 mg, 1.1 mmol) was added, and the mixture left to stir overnight. The yellow suspension was diluted with 35 mL water and the prec. Filtered to provide compound 243 (430 mg, >100%. 400 MHz ¹H NMR (DMSO): 8.05 (s, 1H), 8.02 (m, 2H), 7.86 (s, 1H), 7.58 (s, 1H), 2.77 (s, 3H), 2.28 (m, 1H), 1.09 (m, 2H), 0.94 (m, 2H); MS[M+H]=335.13.

### Preparation of Compound 244

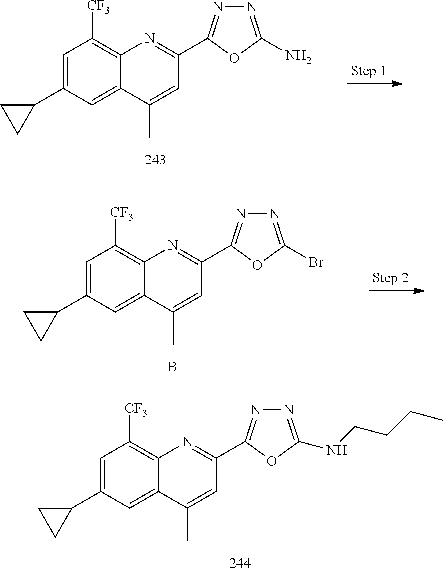

### Step 1

Compound 243 (250 mg, 0.748 mmol), suspended in acetonitrile (9 mL), was treated with copper (II) bromide (250 mg, 1.12 mmol), followed by t-butyl nitrite (180 μL, 1.50 mmol). Reaction mixture was stirred at rt for 2 h and then concentrated. The residue was diluted with EtOAc, and washed with water. The organic layer was concentrated to give the crude compound B as a yellowish-brown solid (260 mg, 87%).

### Step 2

Compound B (130 mg, 0.327 mmol), suspended in THF (3 mL), was treated with n-butyl amine (50 μL, 0.490 mmol). The reaction mixture was heated at 50° C. for 2 h. It was then cooled to rt and concentrated. The residue was suspended in DMF and filtered through a syringe filter before purification by prep HPLC to give an off-white solid 244 (50 mg, 39%)

¹H NMR (400 MHz, DMSO-d6) δ 8.13 (t, J=5.6 Hz, 1H), 8.06 (s, 1H), 8.02 (s, 1H), 7.86 (s, 1H), 3.25 (dd, J=12.8, 6.9 Hz, 2H), 2.75 (d, J=0.8 Hz, 3H), 2.27 (t, J=6.7 Hz, 1H), 1.54 (dd, J=14.7, 7.6 Hz, 2H), 1.34 (dd, J=15.0, 7.4 Hz, 2H), 1.13-1.06 (m, 2H), 0.94 (dt, J=6.9, 4.5 Hz, 2H), 0.88 (t, J=7.4 Hz, 3H); 19F NMR (376.1 MHz) δ −58.82, −75.01 (TFA salt); MS [M+H]⁺=391.2; LC/MS RT=2.57 min.

### Preparation of Compounds 245-262

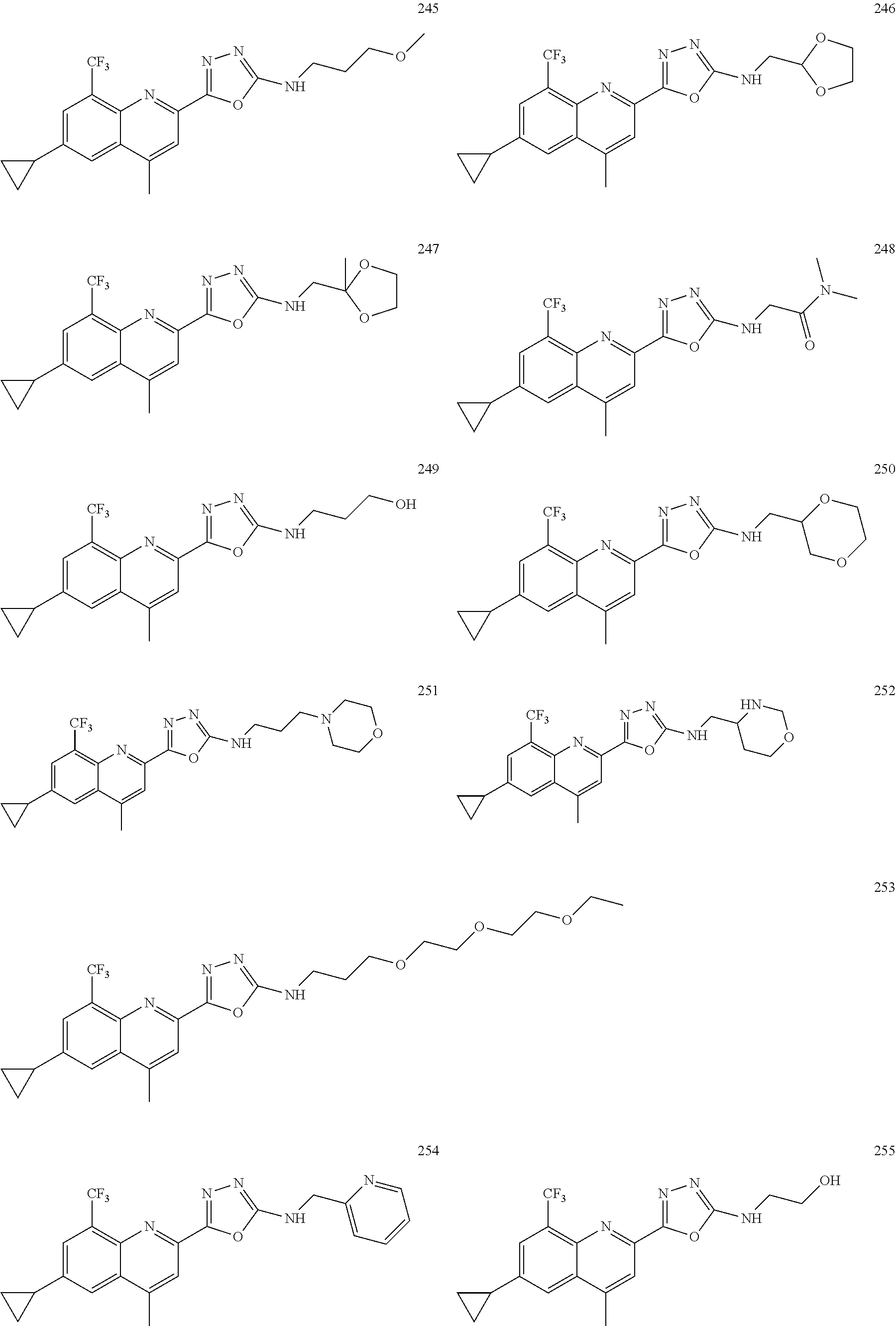

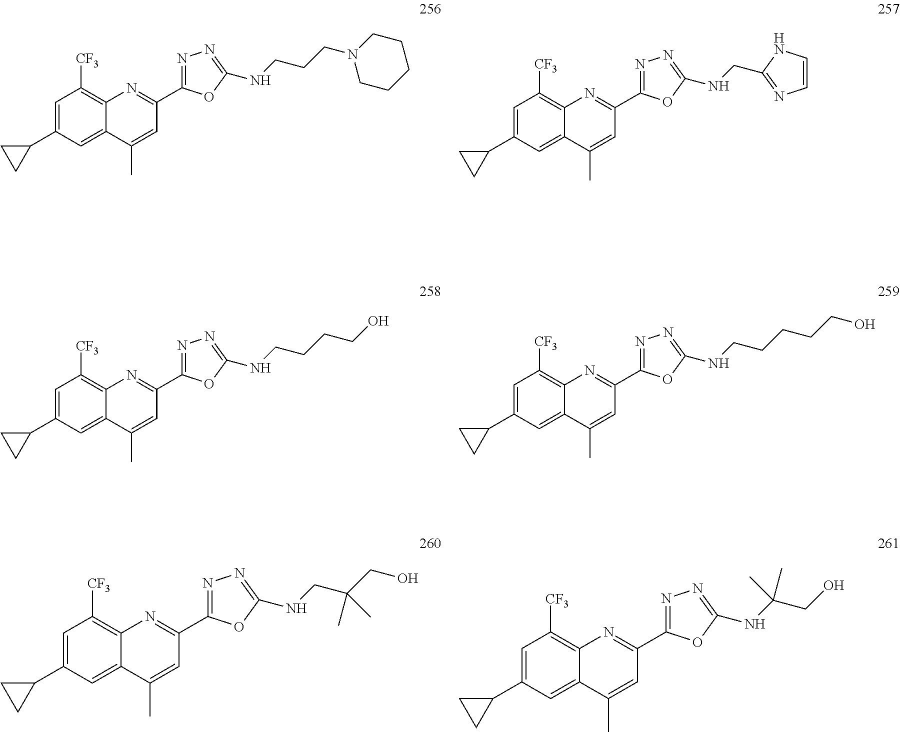

The compounds in the example were made according to procedures described in example compound 244.

245: ¹H NMR (400 MHz, DMSO-d6) δ 8.18 (t, J=5.6 Hz, 1H), 8.08 (s, 1H), 8.04 (s, 1H), 7.88 (s, 1H), 3.34 (m, 4H), 3.22 (s, 3H), 2.77 (d, J=0.8 Hz, 3H), 2.30 (m, 1H), 1.82 (dd, J=14.7, 7.6 Hz, 2H), 1.14-1.10 (m, 2H), 0.98 (dt, J=6.9, 4.5 Hz, 2H); ¹⁹F NMR (376.1 MHz) δ −58.81, −74.98 (TFA salt); MS [M+H]⁺=407.2; LC/MS RT=2.42 min.

246: ¹H NMR (400 MHz, DMSO-d6) δ 8.34 (t, J=6.1 Hz, 1H), 8.06 (s, 1H), 8.02 (d, J=1.8 Hz, 1H), 7.86 (s, 1H), 5.03 (t, J=4.3 Hz, 1H), 3.96-3.89 (m, 2H), 3.83-3.77 (m, 2H), 3.39 (dd, J=6.1, 4.3 Hz, 2H), 2.76 (s, 3H), 2.27 (td, J=8.3, 4.1 Hz, 1H), 1.13-1.04 (m, 2H), 0.98-0.89 (m, 2H); ¹⁹F NMR (376.1 MHz) δ −58.82, −74.98 (TFA salt); MS [M+H]⁺=421.2; LC/MS RT=2.38 min.

247: ¹H NMR (400 MHz, DMSO-d6) δ 8.28 (t, J=6.6 Hz, 1H), 8.05 (s, 1H), 8.02 (s, 1H), 7.86 (s, 1H), 3.94 (dt, J=13.0, 8.4 Hz, 2H), 3.89-3.83 (m, 2H), 3.36 (d, J=6.5 Hz, 2H), 2.75 (d, J=0.8 Hz, 3H), 2.28 (d, J=8.2 Hz, 1H), 1.30 (s, 3H), 1.14-1.04 (m, 2H), 0.94 (dt, J=6.8, 4.5 Hz, 2H); ¹⁹F NMR (376.1 MHz) δ −58.82, −74.98 (TFA salt); MS [M+H]⁺=421.2; LC/MS RT=2.38 min.

248: ¹H NMR (400 MHz, DMSO-d6) δ 8.29-8.21 (m, 1H), 8.06 (s, 1H), 8.02 (s, 1H), 7.87 (s, 1H), 4.12 (d, J=6.4 Hz, 2H), 2.98 (s, 3H), 2.83 (s, 3H), 2.76 (s, 3H), 2.33-2.23 (m, 1H), 1.10 (d, J=6.1 Hz, 2H), 0.94 (d, J=4.9 Hz, 2H); ¹⁹F NMR (376.1 MHz) δ −58.83, −74.90 (TFA salt); MS [M+H]⁺=420.3; LC/MS RT=2.30 min.

249: ¹H NMR (400 MHz, DMSO-d6) δ 8.10 (s, 1H), 8.06 (s, 1H), 8.02 (s, 1H), 7.86 (s, 1H), 3.47 (t, J=6.2 Hz, 2H), 3.31 (d, J=6.4 Hz, 2H), 2.75 (s, 3H), 2.27 (m, 1H), 1.77-1.66 (m, 2H), 1.09 (d, J=8.2 Hz, 2H), 0.93 (d, J=4.8 Hz, 2H); ¹⁹F NMR (376.1 MHz) δ −58.80, −75.08 (TFA salt); MS [M+H]⁺=393.3; LC/MS RT=2.22 min.

250: ¹H NMR (400 MHz, DMSO-d6) δ 8.27 (s, 1H), 8.06 (s, 1H), 8.02 (s, 1H), 7.86 (s, 1H), 3.82-3.65 (m, 3H), 3.63-3.49 (m, 2H), 3.46 (d, J=8.2 Hz, 1H), 3.33-3.21 (m, 3H), 2.75 (s, 3H), 2.27 (s, 1H), 1.09 (d, J=8.6 Hz, 2H), 0.94 (d, J=6.1 Hz, 2H); ¹⁹F NMR (376.1 MHz) δ −58.80, −75.09 (TFA salt); MS [M+H]⁺=435.3; LC/MS RT=2.35 min.

251: ¹H NMR (400 MHz, DMSO-d6) δ 9.67-9.53 (m, 1H), 8.28 (s, 1H), 8.07 (s, 1H), 8.03 (s, 1H), 7.87 (s, 1H), 3.95 (d, J=13.0 Hz, 2H), 3.59 (d, J=12.5 Hz, 2H), 3.42 (d, J=11.2 Hz, 2H), 3.35 (d, J=5.8 Hz, 2H), 3.20 (s, 2H), 3.03 (s, 2H), 2.77 (d, J=7.5 Hz, 3H), 2.27 (s, 1H), 1.98 (s, 2H), 1.10 (d, J=6.2 Hz, 2H), 0.93 (d, J=5.1 Hz, 2H); ¹⁹F NMR (376.1 MHz) δ −58.62, −74.68 (TFA salt); MS [M+H]⁺=462.2; LC/MS RT=2.05 min.

252: ¹H NMR (400 MHz, DMSO-d6) δ 9.12 (s, 1H), 8.88 (s, 1H), 8.47 (s, 1H), 8.09 (s, 1H), 8.03 (s, 1H), 7.88 (s, 1H), 3.98 (d, J=9.1 Hz, 1H), 3.85 (d, J=11.8 Hz, 1H), 3.64 (t, J=10.9 Hz, 1H), 3.52 (m, 4H), 3.23 (s, 1H), 3.08 (s, 1H), 2.77 (s, 3H), 2.28 (s, 1H), 1.10 (d, J=8.5 Hz, 2H), 0.94 (d, J=5.0 Hz, 2H); ¹⁹F NMR (376.1 MHz) δ −58.80, −74.31 (TFA salt); MS [M+H]⁺=434.3; LC/MS RT=1.98 min.

253: ¹H NMR (400 MHz, DMSO-d6) δ 8.11 (t, J=5.5 Hz, 1H), 8.06 (s, 1H), 8.02 (s, 1H), 7.85 (s, 1H), 3.51-3.26 (m, 14H), 2.75 (s, 3H), 2.27 (s, 1H), 1.85-1.75 (m, 2H), 1.09 (d, J=8.3 Hz, 2H), 1.04 (t, J=7.0 Hz, 3H), 0.93 (d, J=5.0 Hz, 2H); ¹⁹F NMR (376.1 MHz) δ −58.80; MS [M+H]⁺=509.3; LC/MS RT=2.45 min.

254: ¹H NMR (400 MHz, DMSO-d6) δ 8.89 (s, 1H), 8.61 (d, J=4.7 Hz, 1H), 8.01 (dd, J=18.9, 9.8 Hz, 3H), 7.87 (s, 1H), 7.63 (d, J=7.8 Hz, 1H), 7.52-7.44 (m, 1H); 4.66 (d, J=5.7 Hz, 2H), 2.75 (s, 3H), 2.26 (td, J=8.3, 4.3 Hz, 1H), 1.15-1.04 (m, 2H), 0.98-0.88 (m, 2H); ¹⁹F NMR (376.1 MHz) δ −58.80, −75.21 (TFA salt); MS [M+H]⁺=426.2; LC/MS RT=2.30 min.

255: ¹H NMR (400 MHz, DMSO-d6) δ 8.14 (s, 1H), 8.06 (s, 1H), 8.02 (s, 1H), 7.86 (s, 1H), 3.55 (t, J=5.9 Hz, 2H), 3.31 (d, J=5.8 Hz, 2H), 2.75 (s, 3H), 2.27 (m, 1H), 1.09 (m, 2H), 0.93 (m, 2H); ¹⁹F NMR (376.1 MHz) δ −58.82, −74.98 (TFA salt); MS [M+H]⁺=379.2; LC/MS RT=2.20 min.

256: ¹H NMR (400 MHz, DMSO-d6) δ 9.03 (s, 1H), 8.28 (t, J=5.7 Hz, 1H), 8.07 (s, 1H), 8.03 (s, 1H), 7.87 (s, 1H), 3.42 (d, J=12.1 Hz, 2H), 3.34 (d, J=6.2 Hz, 2H), 3.11 (s, 2H), 2.84 (d, J=11.0 Hz, 2H), 2.77 (d, J=8.2 Hz, 3H), 2.27 (s, 1H), 1.97 (s, 2H), 1.78 (d, J=14.2 Hz, 2H), 1.70-1.49 (m, 3H), 1.34 (d, J=11.9 Hz, 1H), 1.15-1.04 (m, 2H), 0.98-0.89 (m, 2H); ¹⁹F NMR (376.1 MHz) δ −58.79, −74.47 (TFA salt); MS [M+H]⁺=460.3; LC/MS RT=2.13 min.

257: ¹H NMR (400 MHz, DMSO-d6) δ 9.05 (s, 1H), 8.08 (s, 1H), 8.04 (s, 1H), 7.89 (s, 1H), 7.59 (s, 2H), 4.81 (d, J=5.6 Hz, 2H), 2.76 (s, 3H), 2.28 (m, 1H), 1.10 (d, J=8.1 Hz, 2H), 0.94 (d, J=5.0 Hz, 2H); ¹⁹F NMR (376.1 MHz) δ −58.79, −74.14 (TFA salt); MS [M+H]⁺=415.3; LC/MS RT=1.99 min.

258: ¹H NMR (400 MHz, DMSO-d6) δ 8.15 (s, 1H), 8.06 (s, 1H), 8.02 (s, 1H), 7.86 (s, 1H), 3.39 (t, J=6.4 Hz, 2H), 3.25 (d, J=6.1 Hz, 2H), 2.75 (s, 3H), 2.27 (s, 1H), 1.66-1.53 (m, 2H), 1.52-1.40 (m, 2H), 1.09 (d, J=8.3 Hz, 2H), 0.93 (d, J=4.8 Hz, 2H); ¹⁹F NMR (376.1 MHz) δ −58.81, −75.21 (TFA salt); MS [M+H]⁺=407.3; LC/MS RT=2.30 min.

259: ¹H NMR (400 MHz, DMSO-d6) δ 8.13 (s, 1H), 8.05 (s, 1H), 8.02 (s, 1H), 7.85 (s, 1H), 3.36 (m, 3H), 3.24 (d, J=6.3 Hz, 2H), 2.75 (s, 3H), 2.27 (s, 1H), 1.62-1.50 (m, 2H), 1.41 (d, J=6.8 Hz, 2H), 1.34 (d, J=7.0 Hz, 2H), 1.09 (d, J=7.7 Hz, 2H), 0.94 (m, 2H); ¹⁹F NMR (376.1 MHz) δ −58.77; MS [M+H]⁺=421.3; LC/MS RT=2.34 min.

260: ¹H NMR (400 MHz, DMSO-d6) δ 11.10 (s, 1H), 10.28 (s, 1H), 9.93 (s, 1H), 8.10 (s, 1H), 8.06 (s, 1H), 7.93 (s, 1H), 4.31 (s, 2H), 3.10 (s, 2H), 2.80 (s, 3H), 2.30 (d, J=5.0 Hz, 1H), 1.13 (m, 2H), 1.03 (s, 6H), 1.00-0.93 (m, 2H); ¹⁹F NMR (376.1 MHz) δ −58.44, −74.27 (TFA salt); MS [M+H]⁺=407.3; LC/MS RT=2.30 min.

261: ¹H NMR (400 MHz, DMSO-d6) δδ 10.85-10.72 (m, 1H), 10.50 (s, 1H), 8.09 (s, 2H), 7.93 (s, 1H), 4.68 (s, 2H), 2.80 (s, 3H), 2.30 (m, 1H), 1.35 (s, 6H), 1.12 (m, 2H), 0.96 (m, 2H); ¹⁹F NMR (376.1 MHz) δ −58.45, −74.33 (TFA salt); MS [M+H]⁺=407.3; LC/MS RT=2.13 min.

### Preparation of Compound 262

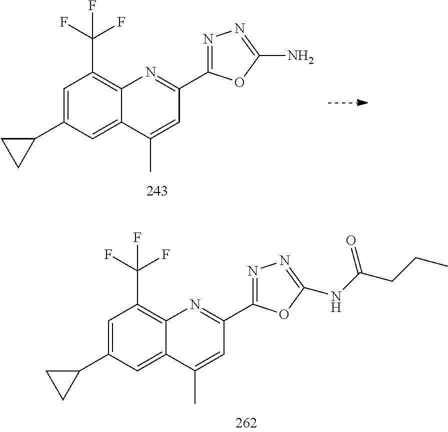

A solution of compound 243 (113 mg, 0.17 mmol) in 3 mL DCM was treated with the acid chloride of butyric acid (50 uL, 0.21 mmol) and pyridine (67 uL, 0.8 uL) and stirred at rt for 30 min. The volatiles were removed in vacuo, and the residue taken up in 3 mL DMF. Purification by RP HPLC provided compound 262. 400 MHz ¹H NMR (DMSO): 11.82 (s, 1H), 8.15 (s, 1H), 8.06 (s, 1H), 7.90 (s, 1H), 2.79 (s, 3H), 2.44 (t, J=8 Hz, 2H), 1.61 (app hextet, J=8 Hz, 2H), 1.10 (m, 2H), 0.96 (m, 2H), 0.94 (t, J=8 Hz, 3H). MS[M+H]=405.35.

### Preparation of Compound 263

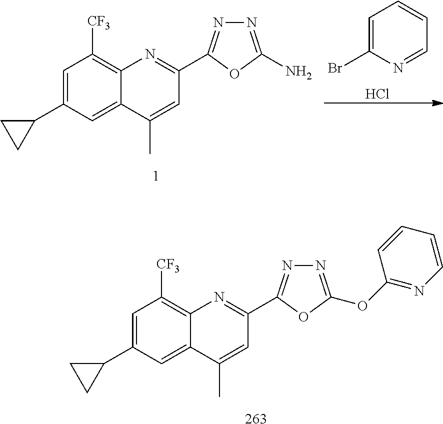

Compound 243 (50 mg, 0.150 mmol) and 2-bromo pyridine (150 μL, 1.50 mmol) were combined in a vial and one drop of conc. HCl solution was added. The reaction mixture was heated at 140° C. for 30 min. After cooling to rt, the reaction was concentrated and the residue was dissolved in DMF, filtered through a syringe filter and purified by prep HPLC to give compound 263 as a white solid (15 mg, 24%).

¹H NMR (400 MHz, DMSO-d6) δ 8.56 (s, 1H), 8.10 (d, J=16.4 Hz, 2H), 8.02 (d, J=8.2 Hz, 1H), 7.91 (d, J=9.6 Hz, 2H), 7.41 (s, 1H), 2.81 (s, 3H), 2.30 (m, 1H), 1.12 (m, 2H), 0.98 (m, 2H); ¹⁹F NMR (376.1 MHz) δ −58.80, −75.16 (TFA salt); MS [M+H]⁺=413.1; LC/MS RT=2.54 min.

### Preparation of Compound 64

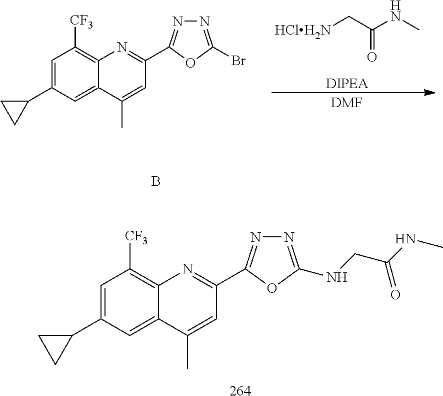

Compound B from this example (100 mg, 0.251 mmol), suspended in DMF (3 mL), was treated with 2-amino-N-methyl acetamide hydrochloride (47 mg, 0.377 mmol), followed by diisopropyl ethylamine (90 μL, 0.503 mmol). The reaction mixture was stirred at rt overnight. It was concentrated and the residue was suspended in DMF and filtered through a syringe filter before purification by prep HPLC to give an off-white solid 264 (20 mg, 20%).

¹H NMR (400 MHz, DMSO-d6) δ 8.44 (s, 1H), 8.07 (s, 1H), 8.03 (s, 1H), 7.99 (s, 1H), 7.87 (s, 1H), 3.82 (d, J=6.2 Hz, 2H), 2.76 (s, 3H), 2.58 (d, J=4.6 Hz, 3H), 2.27 (m, 1H), 1.09 (m, 2H), 0.94 (m, 2H); ¹⁹F NMR (376.1 MHz) δ −58.80, −75.16 (TFA salt); MS [M+H]⁺=406.2; LC/MS RT=2.20 min.

### Preparation of Compounds 265-270

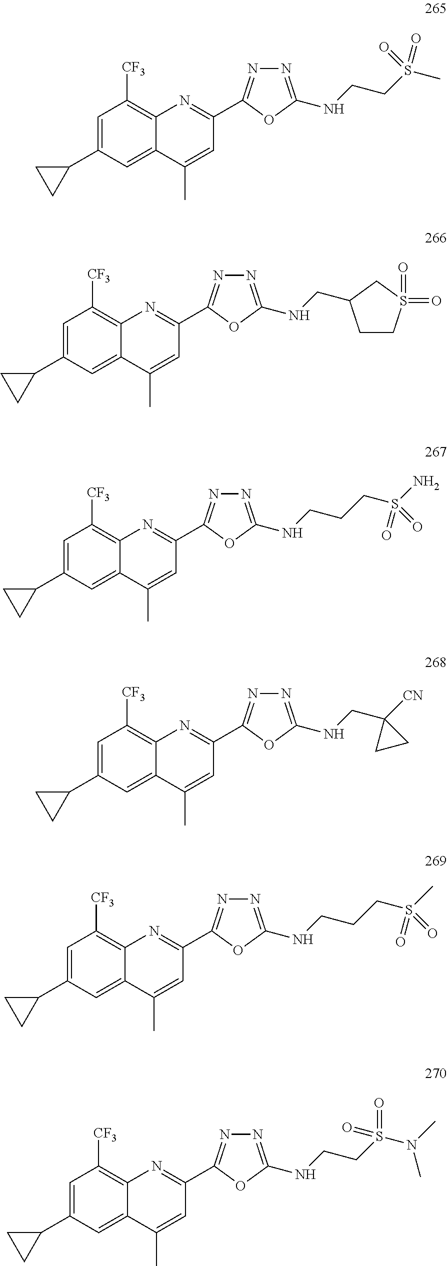

These compounds were made according to procedures described previously.

265: ¹H NMR (400 MHz, DMSO-d6) δ 8.45 (s, 1H), 8.08 (s, 1H), 8.03 (s, 1H), 7.86 (s, 1H), 3.69 (d, J=6.2 Hz, 2H), 3.45 (t, J=6.8 Hz, 2H), 3.03 (s, 3H), 2.76 (s, 3H), 2.27 (m, 1H), 1.10 (m, 2H), 0.94 (m, 2H); ¹⁹F NMR (376.1 MHz) δ −58.78, −74.93 (TFA salt); MS [M+H]⁺=441.3; LC/MS RT=2.25 min.

266: ¹H NMR (400 MHz, DMSO-d6) δ 8.35 (s, 1H), 8.07 (s, 1H), 8.03 (s, 1H), 7.87 (s, 1H), 3.39 (t, J=6.3 Hz, 2H), 3.24 (m, 1H), 3.19 (m, 1H), 3.07 (m, 1H), 2.91-2.82 (m, 1H), 2.75 (s, 3H), 2.72 (m, 1H), 2.27 (m, 2H), 1.91-1.79 (m, 1H), 1.09 (d, J=8.5 Hz, 2H), 0.94 (d, J=5.0 Hz, 2H); ¹⁹F NMR (376.1 MHz) δ 58.79, −75.07 (TFA salt); MS [M+H]⁺=467.3; LC/MS RT=2.37 min.

267: ¹H NMR (400 MHz, DMSO-d6) δ 8.25 (s, 1H), 8.07 (s, 1H), 8.02 (s, 1H), 7.86 (s, 1H), 3.39 (d, J=6.1 Hz, 2H), 3.24-3.13 (m, 2H), 2.75 (s, 3H), 2.27 (m, 1H), 2.00 (s, 2H), 1.09 (m, 2H), 0.95 (m, 2H); ¹⁹F NMR (376.1 MHz) δ −58.77, −75.04 (TFA salt); MS [M+H]⁺=455.3; LC/MS RT=2.24 min.

268: ¹H NMR (400 MHz, DMSO-d6) δ 8.70 (s, 1H), 8.07 (s, 1H), 8.03 (s, 1H), 7.87 (s, 1H), 3.44 (d, J=6.0 Hz, 2H), 2.76 (s, 3H), 2.28 (s, 1H), 1.24 (s, 2H), 1.17 (s, 2H), 1.10 (d, J=6.5 Hz, 2H), 0.95 (s, 2H); ¹⁹F NMR (376.1 MHz) δ −58.81, −75.07 (TFA salt); MS [M+H]⁺=414.3; LC/MS RT=2.48 min.

269: ¹H NMR (400 MHz, DMSO-d6) δ 8.25 (t, J=5.6 Hz, 1H), 8.07 (s, 1H), 8.02 (s, 1H), 7.86 (s, 1H), 3.39 (q, J=6.7 Hz, 2H), 3.25-3.14 (m, 2H), 2.95 (s, 3H), 2.76 (s, 3H), 2.28 (d, J=8.2 Hz, 1H), 2.01 (dd, J=14.9, 7.1 Hz, 2H), 1.15-1.04 (m, 2H), 0.97-0.89 (m, 2H); ¹⁹F NMR (376.1 MHz) δ −58.79, −75.17 (TFA salt); MS [M+H]⁺=455.3; LC/MS RT=2.22 min.

270: ¹H NMR (400 MHz, DMSO-d6) δ 8.42 (s, 1H), 8.07 (s, 1H), 8.03 (s, 1H), 7.87 (s, 1H), 3.63 (s, 2H), 3.37 (d, J=7.3 Hz, 2H), 2.77 (s, 9H), 2.34-2.22 (m, 1H), 1.10 (m, 2H), 0.95 (m, 2H); ¹⁹F NMR (376.1 MHz) δ −58.82, −74.64 (TFA salt); MS [M+H]⁺=470.1; LC/MS RT=2.49 min.

### Preparation of Compound 271

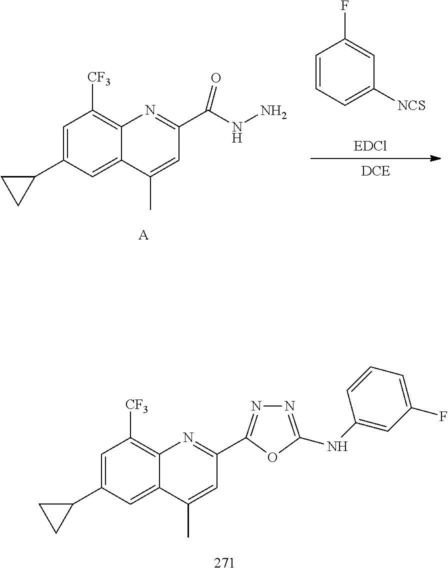

Compound A (100 mg, 0.324 mmol), suspended in DCE (3 mL), was treated with 3-fluorophenyl isothiocyanate (50 mg, 0.323 mmol). The reaction mixture was stirred at 55° C. for 2 h. It was then cooled to it and EDCl (186 mg, 0.971 mmol) was then added. The reaction mixture was heated at 55° C. for another 3 h. It was concentrated and the residue was suspended in DMF and filtered through a syringe filter before purification by prep HPLC to give a white solid 271 (11 mg, 9%).

¹H NMR (400 MHz, DMSO-d6) δ 11.18 (s, 1H), 8.17 (s, 1H), 8.06 (s, 1H), 7.90 (s, 1H), 7.59 (d, J=12.1 Hz, 1H), 7.44-7.34 (m, 2H), 6.84 (s, 1H), 2.79 (s, 3H), 2.29 (s, 1H), 1.11 (d, J=8.0 Hz, 2H), 0.96 (d, J=6.7 Hz, 2H); ¹⁹F NMR (376.1 MHz) δ −58.80, −75.16 (TFA salt); MS [M+H]⁺=429.3; LC/MS RT=2.65 min.

### Preparation of Compound 272

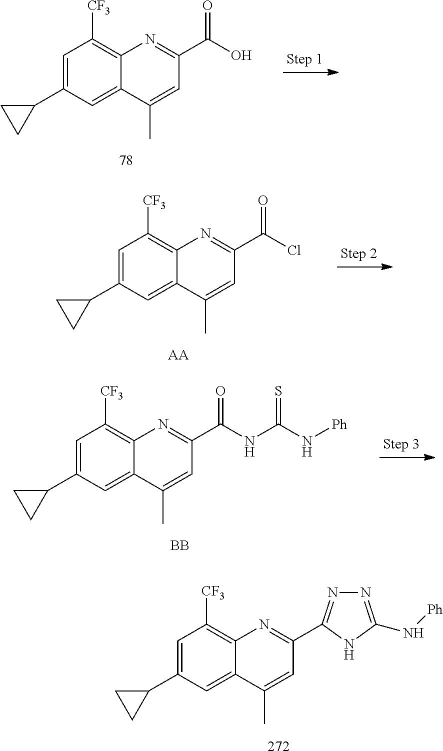

### Step 1

Compound 78 (100 mg, 0.678 mmol) was dissolved in DCM (5 mL) and treated with oxalyl chloride (120 μL, 1.356 mmol) followed by 50 μL of DMF. After 30 minutes, the reaction mixture was concentrated to give crude compound AA as a yellow solid.

### Step 2

Compound AA (50 mg, 0.160 mmol), suspended in dioxane (1.5 mL) was treated with phenylthiourea (24 mg, 0.160 mmol) followed by triethylamine (22 μL, 0.160 mmol). The reaction mixture was heated at 115° C. for 1 h. After cooling to rt, the reaction mixture was filtered and the filtrate was concentrated to give crude compound BB as a light-yellow crystalline solid (70 mg, 100%).

### Step 3

Compound BB (68 mg, 0.159 mmol) was dissolved in chloroform (1.5 mL) and treated with hydrazine hydrate (25 μL, 0.793 mmol). The reaction mixture was heated at 67° C. for 1.5 h. After cooling to rt, the reaction mixture was concentrated. The residue was suspended in DMF and filtered through a syringe filter before purification by prep HPLC to give 272 as an off-white solid (3 mg, 4%).

¹H NMR (400 MHz, DMSO-d6) δ 8.05 (d, J=12.5 Hz, 2H), 7.85 (s, 1H), 7.56 (s, 2H), 7.24 (s, 2H), 6.86-6.76 (m, 1H), 2.78 (s, 3H), 2.36-2.22 (m, 1H), 1.10 (s, 2H), 0.95 (s, 2H); ¹⁹F NMR (376.1 MHz) δ −74.97 (TFA salt); MS [M+H]⁺=410.3; LC/MS RT=2.69 min.

### Example 30

### Preparation of Compound 273

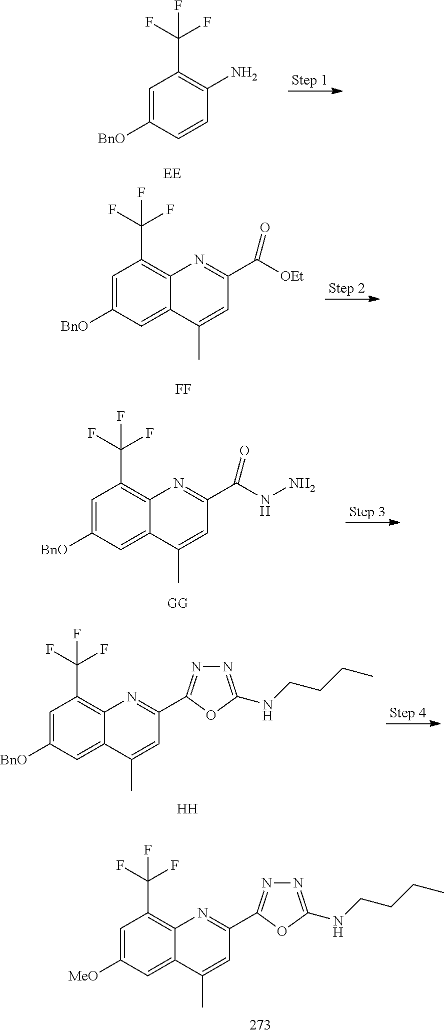

### Step 1

Compound EE was converted to compound FF using the procedures described in example I, Steps 1 thru 5. MS [M+H]⁺=390.10

### Step 2

Compound FF was converted to compound GG using the procedure described in Example IX, Step 1.

MS [M+H]⁺=392.10.

### Step 3

Compound GG was converted to compound HH using the procedure described previously. MS[M+H]=457.18.

### Step 4

A solution of compound HH (457 mg, 1 mmol) in 6 mL TFA was treated with 3 mmol of thioanisole and heated at 40 C for 16 h. Volatiles were removed in vacuo and the residue crystallized refluxing DCM to provide 250 mg of the phenol intermediate as a white solid. 100 mg (0.272 mmol) of the phenol was suspended in MeOH and treated with potassium carbonate (47 mg, 0.34 mmol) and MeI (27 uL, 0.34 mmol). After stirring for 1 h at 55 C The reaction was purified by prep HPLC to provide Compound 273 (27.1 mg, 24% Yield). 400 MHz ¹H NMR (DMSO): 400 MHz ¹H NMR (DMSO): 11.82 (s, 1H) 8.15 (s, 1H), 8.06 (s, 1H), 7.90 (s, 1H), 2.79 (s, 1H), 2.44 (m, 2H), 2.32 (m, 1H), 1.60 (hex, J=8 Hz, 2H), 1.10 (m, 2H), 0.96 (m, 2H), 0.94 (t, J=8 Hz, 3H); ¹⁹F NMR (376.1 MHz) δ −58.24, −75.3 (s); MS[M+H]=381.14.

### Preparation of Compound 274

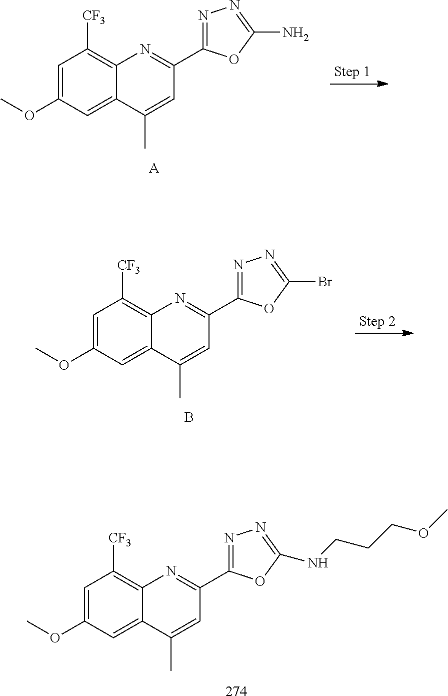

### Step 1

Compound A (1.60 g, 4.93 mmol), suspended in acetonitrile (60 mL), was treated with copper (II) bromide (1.65 g, 7.41 mmol), followed by t-butyl nitrite (1.20 mL, 9.88 mmol). Reaction mixture was stirred at it for 2 h and then concentrated. The residue was diluted with EtOAc and washed with water. The organic layer was concentrated to give the crude compound 5 as a yellowish-brown solid (1.60 g, 84%).

### Step 2

Compound B (100 mg, 0.258 mmol), suspended in THF (3 mL), was treated with 3-methoxypropyl amine (35 mg, 0.387 mmol). The reaction mixture was stirred at it overnight and concentrated. The residue was suspended in DMF and filtered through a syringe filter before purification by prep HPLC to give an off-white solid 274 (33 mg, 32%).

¹H NMR (400 MHz, DMSO-d6) δ 8.11 (s, 1H), 8.07 (s, 2H), 7.80 (s, 1H), 7.63 (s, 1H), 3.99 (s, 3H), 3.38 (t, J=6.2 Hz, 2H), 3.30 (d, J=5.9 Hz, 2H), 3.21 (s, 3H), 2.75 (s, 3H), 1.80 (t, J=6.5 Hz, 2H); ¹⁹F NMR (376.1 MHz) δ −59.20, −74.67 (TFA salt); MS [M+H]⁺=397.2; LC/MS RT=2.33 min.

### Preparation of Compounds 275-279

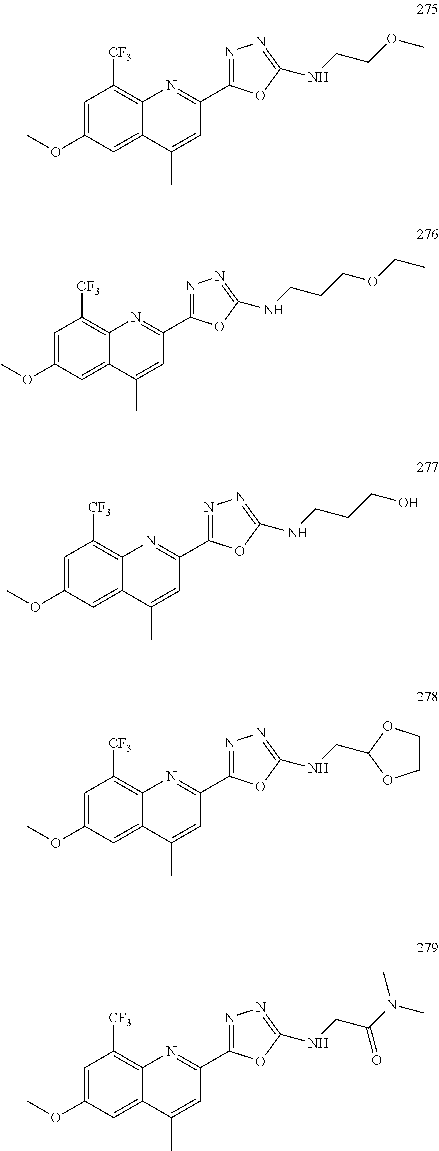

The compounds in the example were made according to procedures described in example 7.

275: ¹H NMR (400 MHz, DMSO-d6) δ 8.20 (t, J=5.7 Hz, 1H), 8.06 (s, 1H), 7.80 (d, J=2.6 Hz, 1H), 7.63 (d, J=2.5 Hz, 1H), 3.99 (s, 3H), 3.50 (t, J=5.6 Hz, 2H), 3.42 (t, J=5.3 Hz, 2H), 3.25 (s, 3H), 2.75 (s, 3H); ¹⁹F NMR (376.1 MHz) δ −59.22, −75.22 (TFA salt); MS [M+H]⁺=383.2; LC/MS RT=2.28 min.

276: ¹H NMR (400 MHz, DMSO-d6) δ 8.19 (s, 1H), 8.07 (s, 1H), 7.81 (s, 1H), 7.64 (s, 1H), 4.00 (d, J=9.6 Hz, 3H), 3.53 (t, J=5.6 Hz, 2H), 3.49-3.35 (m, 4H), 2.75 (s, 3H), 1.08 (t, J=7.0 Hz, 3H); ¹⁹F NMR (376.1 MHz) δ −59.21, −75.14 (TFA salt); MS [M+H]⁺=397.2; LC/MS RT=2.34 min.

277: ¹H NMR (400 MHz, DMSO-d6) δ 8.07 (s, 2H), 7.80 (s, 1H), 7.63 (s, 1H), 3.99 (s, 3H), 3.47 (t, J=6.2 Hz, 2H), 3.30 (d, J=6.3 Hz, 2H), 2.75 (s, 3H), 1.78-1.66 (m, 2H); ¹⁹F NMR (376.1 MHz) δ −59.19, −75.21 (TFA salt); MS [M+H]⁺=383.2; LC/MS RT=2.13 min.

278: ¹H NMR (400 MHz, DMSO-d6) δ 8.31 (s, 1H), 8.06 (s, 1H), 7.80 (s, 1H), 7.63 (s, 1H), 5.03 (s, 1H), 3.99 (s, 3H), 3.92 (t, J=7.0 Hz, 2H), 3.80 (t, J=6.8 Hz, 2H), 3.43-3.35 (m, 2H), 2.75 (s, 3H); ¹⁹F NMR (376.1 MHz) δ −59.20, −75.10 (TFA salt); MS [M+H]⁺=411.1; LC/MS RT=2.26 min.

279: ¹H NMR (400 MHz, DMSO-d6) δ 8.22 (s, 1H), 8.07 (s, 1H), 7.81 (s, 1H), 7.64 (s, 1H), 4.12 (d, J=6.2 Hz, 2H), 3.99 (s, 3H), 2.98 (s, 3H), 2.83 (s, 3H), 2.75 (s, 3H); ¹⁹F NMR (376.1 MHz) δ −59.16, −74.89 (TFA salt); MS [M+H]⁺=410.2; LC/MS RT=2.18 min.

### Preparation of Compound 280

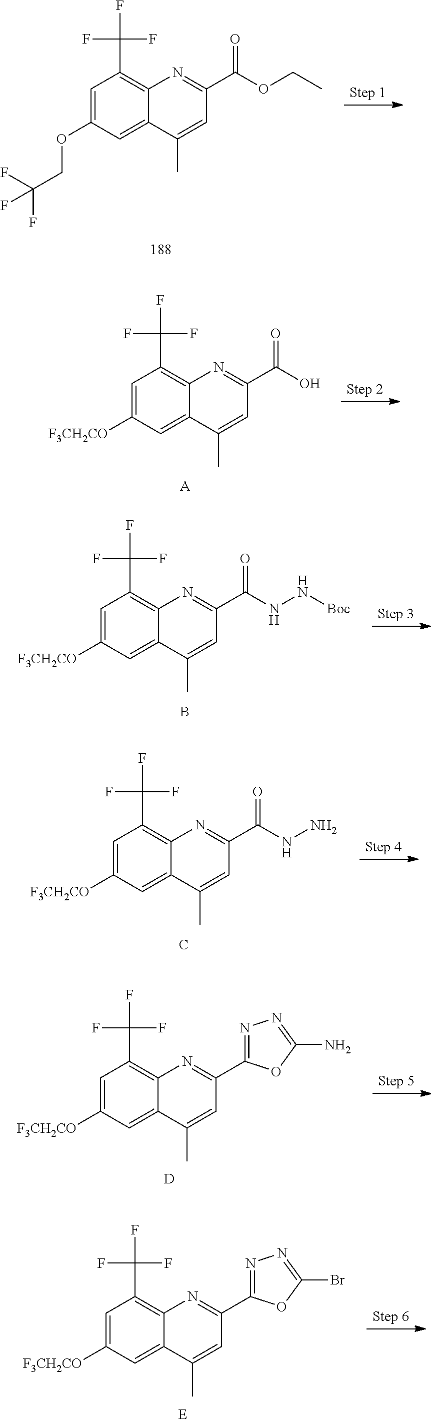

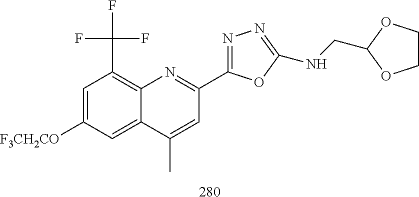

### Step 1

Compound 188 (2.87 g, 7.53 mmol) was dissolved in 100 mL of THF/MeOH (1:1) and treated dropwise with LiOH hydrate (822 mg, 19.6 mmol). The hydrolysis was complete after 17 min. After concentrating the reaction was portioned between ethyl acetate and water containing dilute aqueous HCl, and reextracted with ethyl acetate and washing of the combined organic phases with water and brine before drying and evaporation to afford compound A.

### Step 2

Compound A (7.53 mmol assumed from step 1), dissolved in DCM (25 mL), was treated with diisopropyl ethylamine (6.5 mL, 37.3 mmol), t-butyl carbazate (2.14 gm, 16.2 mmol), followed by HATU (5.37 g, 14.1 mmol). The reaction mixture was stirred at ambient temperature for 2 h and then diluted into saturated aqueous NaHCO₃, extracted with ethyl acetate, washing of the organic phases with water and brine before drying and evaporating. Purification was accomplished via flash column chromatography (silica gel) to give compound B (2.47 g, 70%—two steps).

### Step 3

Compound B (2.47 g) was dissolved in DCM (54 mL), treated with TFA (10 mL) and the resulting yellow solution was stirred at ambient temperature for 1.5 h before diluting with water and extracting with ethyl acetate (to float). The organic phase was washed with water and brine, dried and concentrated to give compound 8 (essentially quantitatively) which was subsequently used as obtained

### Step 4

Compound C (1.96 g, 5.34 mmol), dissolved in dioxane (140 mL), was treated with cyanogen bromide (565 mg, 6.39 mmol) dissolved in dioxane (10 mL), and sodium bicarbonate (677 mg, 8.01 mmol) dissolved in water (8-10 mL). The reaction mixture was stirred at ambient temperature overnight, concentrated, partitioned between ethyl acetate and water. The organic phase was washed with water and brine, dried, evaporated and treated with high vacuum, affording compound D (2.18 g).

### Step 5

Compound D (796 mg, 2.03 mmol), suspended in acetonitrile (30 mL), was treated with copper (II) bromide (960 g, 4.3 mmol), followed by t-butyl nitrite (0.48 μL, 4.06 mmol). Reaction mixture was stirred at ambient temperature for 1.5 h and then concentrated. The residue was diluted with EtOAc, washed with water, brine and dried. The filtered organic layer was concentrated in vacuo to give the crude compound E (749 mg). as a yellowish-brown solid (310 mg, 86%).

### Step 6

Compound E (242 mg, 0.53 mmol) dissolved in DMF (3 mL), was treated with 2-aminomethyl 1,3-dioxolane (117.5 mg, 1.13 mmol). The reaction mixture was stirred at ambient temperature overnight. Purification was accomplished via preparative HPLC to afford compound 280, 150.5 mg.

¹H NMR (400 MHz, dmso) δ 8.34 (t, J=6.1 Hz, 1H), 8.10 (s, 1H), 7.93 (d, J=2.6 Hz, 1H), 7.86 (d, J=2.7 Hz, 1H), 5.06 (dt, J=8.6, 6.5 Hz, 3H), 3.92 (dd, J=8.7, 5.1 Hz, 2H), 3.80 (dd, J=8.7, 5.1 Hz, 2H), 3.40 (dd, J=5.8, 4.5 Hz, 2H), 2.76 (s, 3H); ¹⁹F NMR (376 MHz, dmso) δ −59.19 (s), −72.88 (t, J=8.8 Hz), −75.26 (s); MS [M+H]⁺=393.14.

### Example 31

### Preparation of Compound 281

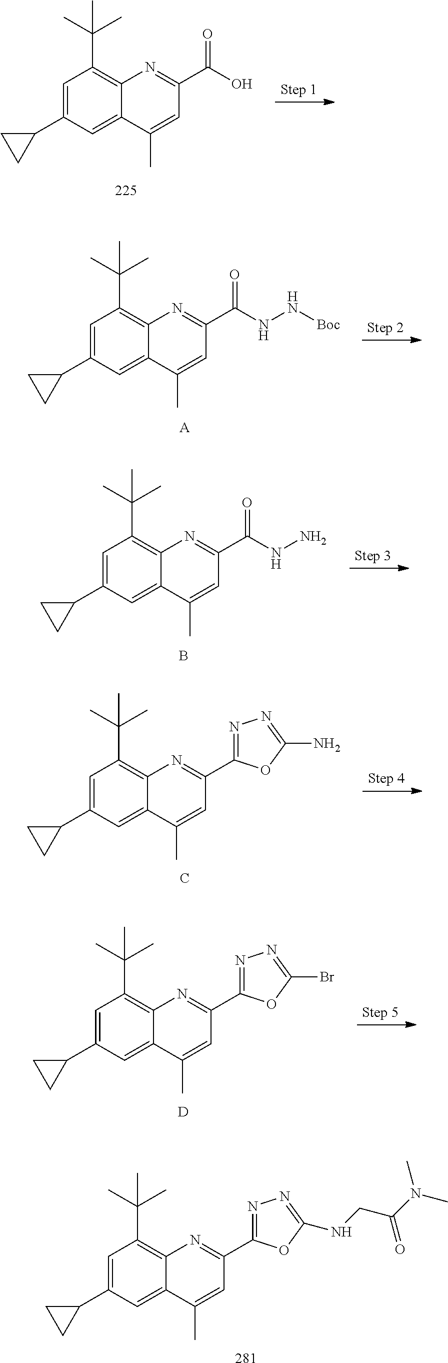

### Step 1

### Compound 225 (800 mg, 2.83 mmol), dissolved in DCM (25 mL), was treated with HATU (2.15 g, 5.65 mmol), t-butyl carbazate (747 mg, 5.65 mmol), followed by N,N-diisopropylethylamine (2 mL, 11.3 mmol). The reaction mixture was stirred at rt for 4 h and then concentrated. The residue was redissolved in EtOAc and washed with 10% sodium citrate solution. The organic layer was concentrated and purified by flash column chromatography to give compound A as a white solid.

### Step 2

Compound A from the previous step was treated with 4 M HCl in dioxane (5 mL) and the resulting yellow solution was stirred at it for 3 h and then concentrated. The residue was redissolved in EtOAc and washed with saturated NaHCO₃ solution. The organic layer was concentrated to give crude compound B as a white solid (540 mg, 64% over 2 steps).

### Step 3

Compound B (540 mg, 1.82 mmol), dissolved in dioxane (50 mL), was treated with cyanogen bromide (193 mg, 1.82 mmol) dissolved in dioxane (5 mL), and sodium bicarbonate (229 mg, 2.73 mmol) dissolved in water (15 mL). The reaction mixture was stirred at it overnight. Water was then added and the reaction mixture was filtered and dried to give crude compound C as a white solid (500 mg, 85%).

### Step 4

Compound C (300 mg, 0.932 mmol), suspended in acetonitrile (10 mL), was treated with copper (II) bromide (312 g, 1.40 mmol), followed by t-butyl nitrite (220 μL, 1.86 mmol). Reaction mixture was stirred at rt for 2 h and then concentrated. The residue was diluted with EtOAc, and washed with water. The organic layer was concentrated to give the crude compound D as a yellowish-brown solid (310 mg, 86%).

### Step 5

Compound D (90 mg, 0.233 mmol), suspended in THF (2 mL), was treated with N,N-dimethyl glycinamide (36 mg, 0.350 mmol). The reaction mixture was stirred at it overnight and concentrated. The residue was suspended in DMF and filtered through a syringe filter before purification by prep HPLC to give an off-white solid 281 (18 mg, 19%).

¹H NMR (400 MHz, DMSO-d6) δ 8.10 (s, 1H), 7.87 (s, 1H), 7.56 (s, 1H), 7.38 (s, 1H), 4.11 (d, J=6.1 Hz, 2H), 2.99 (s, 3H), 2.82 (s, 3H), 2.67 (s, 3H), 2.13 (s, 1H), 1.62 (s, 9H), 1.03 (d, J=8.3 Hz, 2H), 0.83 (s, 2H); ¹⁹F NMR (376.1 MHz) δ −74.97 (TFA salt); MS [M+H]⁺=408.3; LC/MS RT=2.44 min.

### Preparation of Compounds 282-285

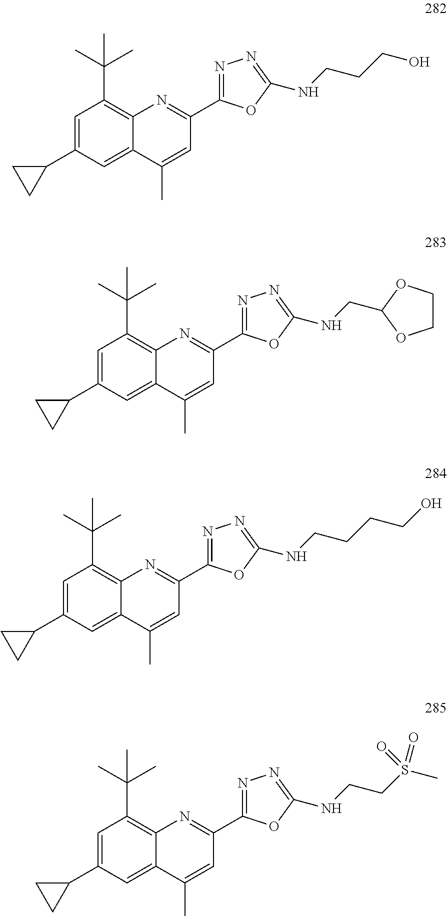

The compounds in the example were made according to procedures described previously.

282: ¹H NMR (400 MHz, DMSO-d6) δ 7.94 (s, 1H), 7.86 (s, 1H), 7.56 (s, 1H), 7.38 (s, 1H), 3.47 (t, J=6.2 Hz, 2H), 3.31 (dd, J=12.9, 6.7 Hz, 2H), 2.67 (s, 3H), 2.13 (m, 1H), 1.74 (dd, J=13.6, 6.6 Hz, 2H), 1.61 (s, 9H), 1.03 (d, J=6.2 Hz, 2H), 0.84 (d, J=6.8 Hz, 2H); ¹⁹F NMR (376.1 MHz) δ −74.80 (TFA salt); MS [M+H]⁺=381.3; LC/MS RT=2.40 min.

283: ¹H NMR (400 MHz, DMSO-d6) δ 8.20 (s, 1H), 7.86 (s, 1H), 7.56 (s, 1H), 7.38 (s, 1H), 5.04 (t, J=4.4 Hz, 1H), 3.92 (t, J=6.9 Hz, 2H), 3.80 (t, J=6.9 Hz, 2H), 3.42-3.35 (m, 2H), 2.67 (s, 3H), 2.13 (m, 1H), 1.61 (s, 9H), 1.04 (d, J=6.4 Hz, 2H), 0.84 (d, J=4.9 Hz, 2H); ¹⁹F NMR (376.1 MHz) δ −75.11 (TFA salt); MS [M+H]⁺=409.3; LC/MS RT=2.54 min.

284: ¹H NMR (400 MHz, DMSO-d6) δ 7.95 (s, 1H), 7.86 (s, 1H), 7.60 (s, 1H), 7.38 (s, 1H), 3.39 (t, J=6.9 Hz, 2H), 3.31 (m, 2H), 2.67 (s, 3H), 2.13 (m, 1H), 1.62 (s, 9H), 1.56 (m, 2H), 1.47 (m, 2H), 1.04 (d, J=6.4 Hz, 2H), 0.84 (d, J=4.9 Hz, 2H); ¹⁹F NMR (376.1 MHz) δ −75.11 (TFA salt); MS [M+H]⁺=395.3; LC/MS RT=2.44 min.

285: ¹H NMR (400 MHz, DMSO-d6) δ 8.26 (s, 1H), 7.89 (s, 1H), 7.57 (s, 1H), 7.38 (s, 1H), 3.73-3.64 (m, 2H), 3.46 (t, J=6.9 Hz, 2H), 3.03 (s, 3H), 2.68 (s, 3H), 2.13 (m, 1H), 1.62 (s, 9H), 1.04 (d, J=6.2 Hz, 2H), 0.84 (d, J=4.9 Hz, 2H); ¹⁹F NMR (376.1 MHz) δ −75.22 (TFA salt); MS [M+H]⁺=429.4; LC/MS RT=2.46 min.

### Example 32

### Preparation of Compound 286

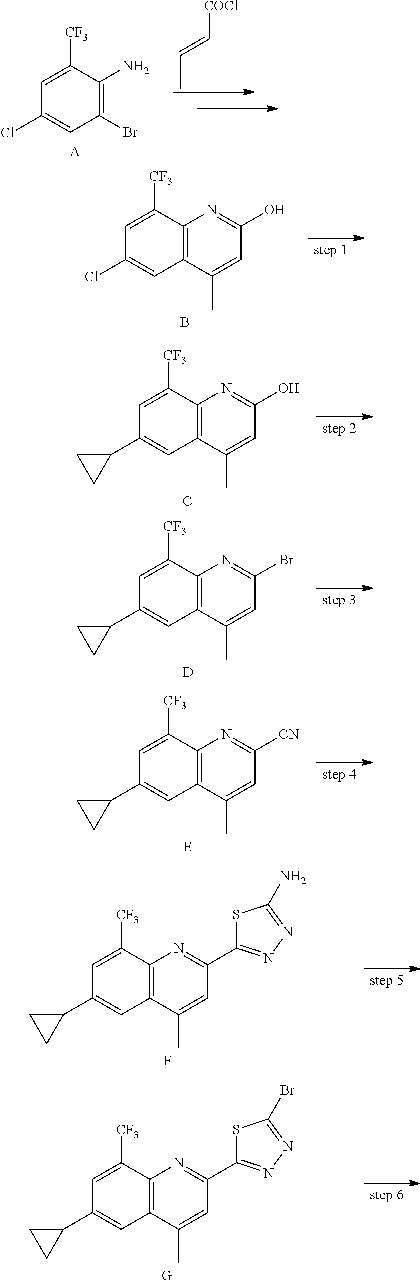

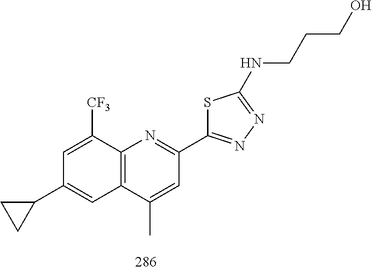

Compound B was obtained from A via amide formation and intramolecular Heck reaction.

### Step 1

Intermediate B (5 g, 19.1 mmol), cyclopropyl potassium trifluoroborate (4.2 g, 28.7 mmol), palladium (II) acetate (0.215 g, 0.95 mmol), Sphos (0.785 g, 1.91 mmol) and K₃PO₄ (8.1 g, 38.2 mmol) were added to a 500 ml round bottom flask. The reaction vessel was placed under vacuum and then refilled with Ar three times. Toluene (60 ml) and water (6 ml) were added to the solid mixture. The reaction vessel was heated to 90° C. with stirring. The reaction was monitored by LC-MS, which showed complete conversion of the starting material after 3 hours. After the flask was cooled to room temperature, the mixture was concentrated under vacuum and re-dissolved in EtOAc. The organic solution was washed successively with concentrated NH₄Cl, water and brine and then dried over Na₂SO₄. The solution was passed through a silica pad and then concentrated under vacuum to give the desired product (2.4 g), which was used in the next step without further purification. MS [M+H]⁺=268.14.

### Step 2

Compound C (1.5 g, 5.61 mmol) was dissolved in 25 ml of 1,4-dioxane in a 100 ml round bottom flask. Phosphorus (V) oxybromide (3.21 g, 11.2 mmol) was added to the flask and the reaction mixture was heated to 80° C. with stirring. LC-MS showed complete conversion to the desired product after 2 hours. After the flask was cooled to room temperature, the mixture was concentrated under vacuum and re-dissolved in EtOAc. The organic solution was washed successively with concentrated NH₄Cl, water and brine and then dried over Na₂SO₄. The solution was concentrated under vacuum and the resulting solid was chromatographed to give the desired product (1.42 g). MS [M+H]⁺=330.35

### Step 3

Compound D (1.42 g. 4.30 mmol) was dissolved in 80 ml of DMF in a 350 ml pressure vessel. Copper (I) cyanide (0.77 g, 8.60 mmol) was added to the mixture. The pressure vessel was sealed and heated to 130° C. with stirring. LC-MS showed complete conversion to the desired product after 3 hours. After the flask was cooled to room temperature, the reaction mixture was further diluted with EtOAc. The organic solution was washed successively with concentrated NH₄Cl, water and brine and then dried over Na₂SO₄. The solution was concentrated under vacuum and the resulting solid was chromatographed to give the desired product (0.722 g). MS [M+H]⁺=277.13.

### Step 4

Compound E (0.722 g, 2.61 g) was dissolved in 25 ml of TFA in a 100 ml round bottom flask. Thiosemicarbazide (0.239 g, 2.61 mmol) was added to the flask and the reaction mixture was heated to 80° C. with stirring. LC-MS showed complete conversion to the desired product after 1 hour. After the flask was cooled to room temperature, the mixture was concentrated under vacuum and re-dissolved in EtOAc. The organic solution was washed successively with concentrated NaHCO₃, water and brine and then dried over Na₂SO₄. The solution was concentrated under vacuum and the resulting solid was chromatographed to give the desired product (0.700 g). MS [M+H]⁺=351.28.

### Step 5

Compound F (0.517 g, 1.48 mmol) was suspended in 15 ml of acetonitrile. Copper (II) bromide (0.496 g, 2.22 mmol) was added and the mixture was stirred for 10 minutes at room temperature. T-butyl nitrite (0.352 ml, 2.95 mmol) was added and the mixture was stirred for 1 hour at room temperature. The solvent was removed under vacuum and the crude mixture was re-dissolved in EtOAc. The organic solution was washed successively with water and brine and then dried over Na₂SO₄. The solution was concentrated under vacuum and the resulting solid (0.420 g) was used in the next step without further purification. MS [M+H]⁺=414.42.

### Step 6

Compound G (0.040 g, 0.096 mmol) was dissolved in 2 ml of DMF in an 8 ml vial. 3-aminopropanol (0.022 g, 0.288 mmol) was added and the mixture was heated to 50° C. The reaction mixture was left stirring overnight. The DMF solution was purified by HPLC to give compound 286 as the TFA salt (23 mg). 400 MHz ¹H NMR (CDCl₃) δ 7.98 (d, 1H), 7.86 (d, 1H), 7.76 (s, 1H), 4.69-4.44 (m, 7H), 3.89 (t, 1H), 3.81-3.57 (m, 2H), 2.92-2.66 (m, 3H), 2.09-2.02 (m, 2H), 2.00 (d, J=8.2 Hz, 2H), 1.34-1.08 (m, 2H), 0.96-0.71 (m, 2H). MS[M+H]=409.16.

### Preparation of Compound 287

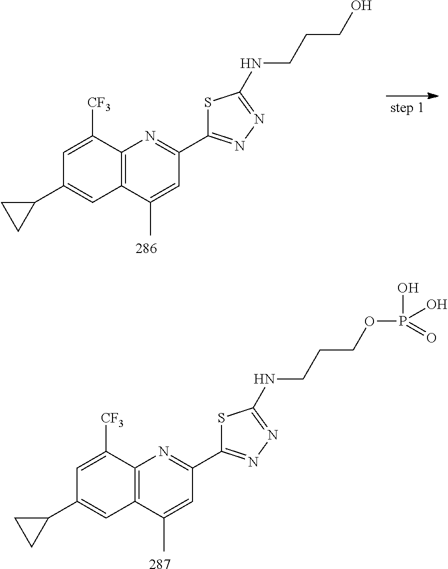

### Step 1

Compound 286 (26 mg, 0.050 mmol) was suspended in 2 ml of DCM. Triethylamine (0.007 ml, 0.050 mmol) was added and the mixture was cooled to 0° C. using an ice bath. Phosphorus (V) oxychloride (7.67 mg, 0.050 mmol) was added dropwise. After the addition was completed, the ice bath was removed and the mixture was stirred at room temperature for 3 hours. The solvent was then removed and the crude was dissolved in 1 ml of THF. To this solution 1 ml of 6 M HCl_{(aq)} was added and the mixture was stirred overnight. The solution was concentrated under vacuum and the crude was re-dissolved in 2 ml of DMF. The solution was purified by HPLC to give compound 287 (5 mg). ¹H-NMR (400 MHz, DMSO-d6) δ 8.09 (s, 1H), 8.00 (s, 1H), 7.83 (s, 1H), 3.90 (s, 2H), 2.75 (s, 3H), 2.64 (s, 2H), 2.29 (s, 2H), 1.90 (s, 1H), 1.09 (s, 2H), 0.93 (s, 2H). ³¹P-NMR δ −0.93 MS [M+H]⁺=489.61.

### Preparation of Compounds 288-304

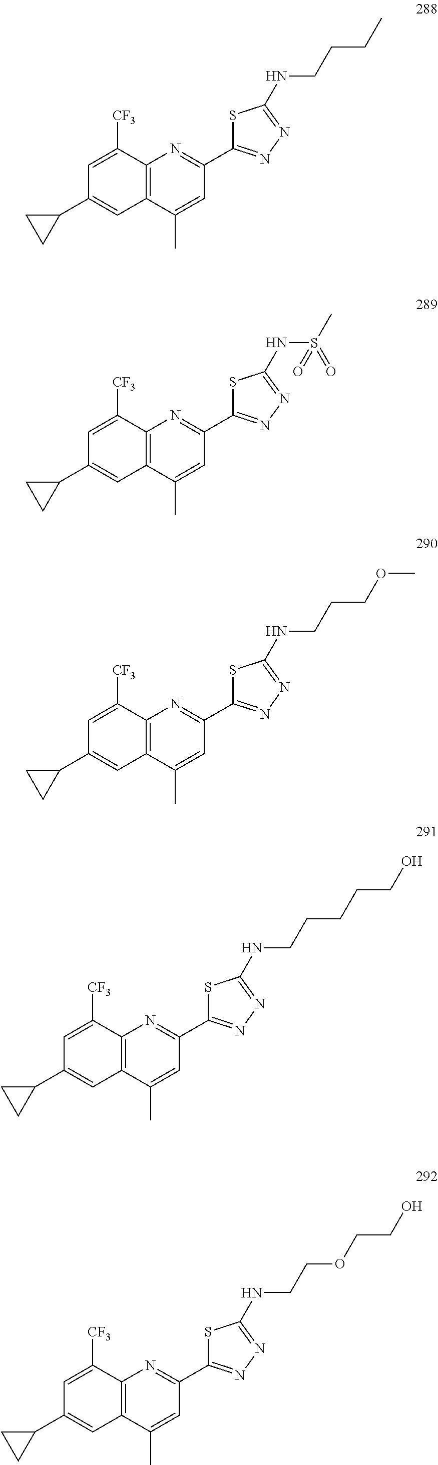

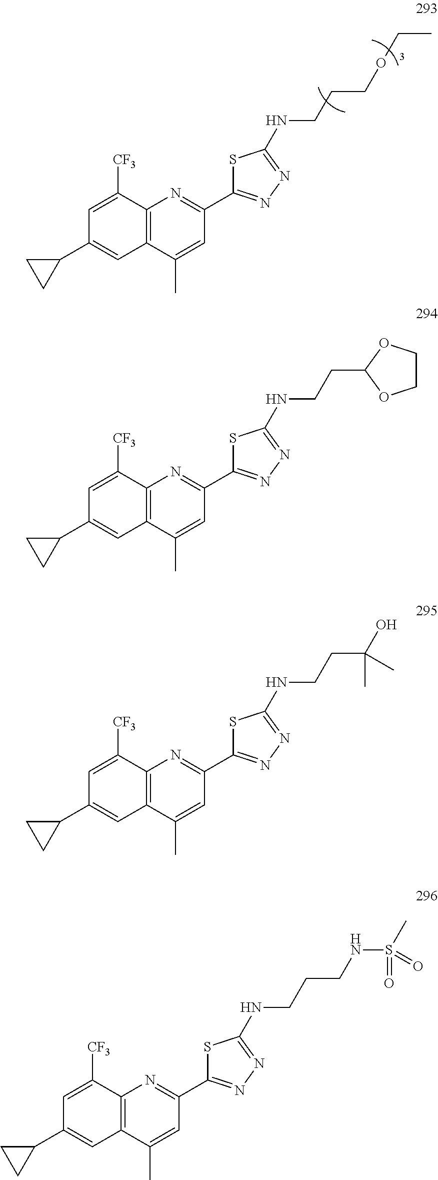

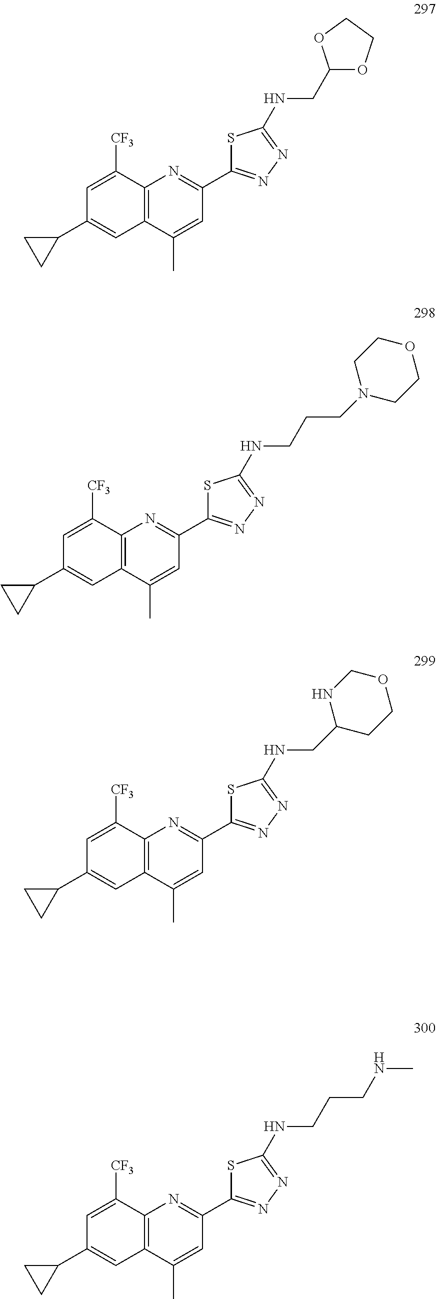

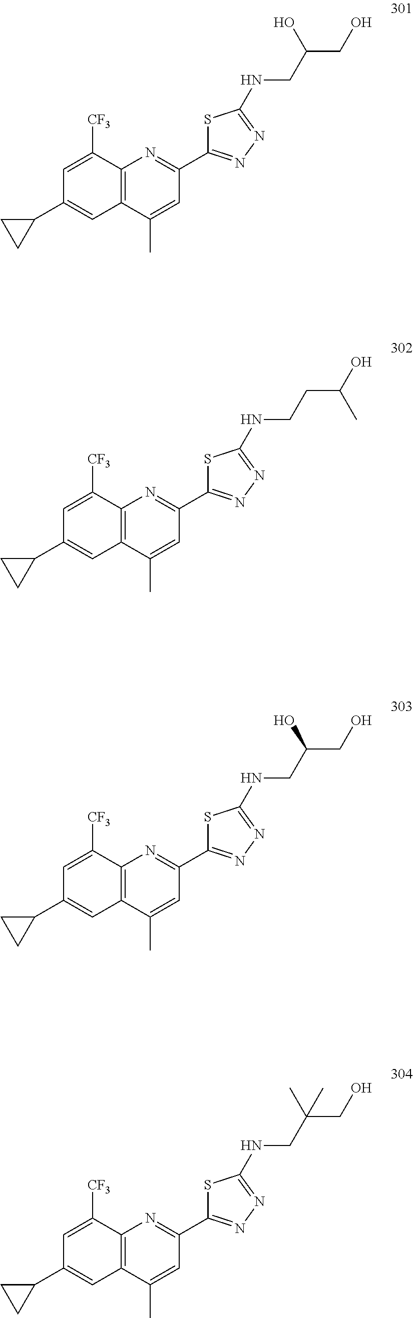

The compounds in the example were made according to the procedure in Step 6 of scheme for example compound 286.

Compound 288: ¹H-NMR (400 MHz, CDCl₃) δ 7.98 (d, 1H), 7.86 (d, 1H), 7.76 (s, 1H), 4.69-4.44 (m, 7H), 3.89 (t, 1H), 3.81-3.57 (m, 2H), 2.92-2.66 (m, 3H), 2.09-2.02 (m, 3H), 2.00 (d, J=8.2 Hz, 1H), 1.34-1.08 (m, 2H), 0.96-0.71 (m, 2H). MS [M+H]⁺=407.24.

Compound 289: ¹H-NMR (400 MHz, DMSO-d6) δ 8.15-7.96 (m, 2H), 7.89 (s, 1H), 3.01 (s, 3H), 2.78 (d, J=0.5 Hz, 3H), 2.37-2.20 (m, 1H), 1.19-1.04 (m, 2H), 1.03-0.83 (m, 2H). MS [M+H]⁺=429.06.

Compound 290: ¹H-NMR (400 MHz, cdcl₃) δ 7.99 (s, 1H), 7.88 (d, J=1.6 Hz, 1H), 7.77 (s, 1H), 3.62 (s, 2H), 3.54 (d, J=5.6 Hz, 2H), 3.39 (s, 2H), 3.33 (s, 1H), 2.78 (d, J=4.3 Hz, 3H), 2.25-2.11 (m, 2H), 2.06 (s, 1H), 1.27-1.09 (m, 2H), 0.89 (dt, J=10.1, 5.0 Hz, 2H). MS [M+H]⁺=423.12.

Compound 291: ¹H-NMR (400 MHz, cdcl₃) δ 7.99 (s, 1H), 7.88 (s, 1H), 7.77 (s, 1H), 4.39 (d, J=6.5 Hz, 1H), 3.71 (dt, J=12.7, 6.0 Hz, 3H), 3.55-3.46 (m, 2H), 2.78 (dd, J=4.7, 0.8 Hz, 3H), 2.28-1.80 (m, 10H), 1.65 (d, J=3.3 Hz, 5H), 1.29-1.07 (m, 2H), 0.89 (td, J=6.6, 3.3 Hz, 2H). MS [M+H]⁺=437.13.

Compound 292: ¹H-NMR (400 MHz, cdcl₃) δ 7.98 (s, 2H), 7.87 (d, J=1.6 Hz, 3H), 7.76 (s, 2H), 3.93 (t, J=5.9 Hz, 2H), 3.89-3.84 (m, 5H), 3.84-3.79 (m, 5H), 3.73-3.61 (m, 12H), 3.60-3.56 (m, 2H), 2.78 (d, J=4.9 Hz, 10H), 2.23-2.08 (m, 1H), 1.24-1.08 (m, 2H), 0.96-0.80 (m, 2H). MS [M+H]⁺=439.10.

Compound 293: ¹H-NMR (400 MHz, cdcl₃) δ 7.97 (s, 1H), 7.86 (d, J=1.6 Hz, 1H), 7.75 (s, 1H), 3.62 (qdd, J=9.2, 6.2, 3.5 Hz, 12H), 3.49 (q, J=7.0 Hz, 2H), 2.76 (s, 3H), 2.15 (ddd, J=13.3, 8.4, 5.0 Hz, 1H), 2.10-1.99 (m, 2H), 1.21-1.10 (m, 5H), 0.87 (tt, J=9.0, 4.5 Hz, 2H). MS [M+H]⁺=525.14.

Compound 294: ¹H-NMR (400 MHz, cdcl₃) δ 7.98 (s, 1H), 7.87 (d, J=1.6 Hz, 1H), 7.76 (d, J=1.3 Hz, 1H), 5.02 (t, J=3.8 Hz, 1H), 4.09-4.02 (m, 2H), 3.92-3.84 (m, 2H), 3.60 (s, 2H), 2.77 (d, J=0.5 Hz, 3H), 2.23-2.09 (m, 3H), 1.22-1.12 (m, 2H), 0.89 (dt, J=6.7, 5.0 Hz, 2H). MS [M+H]⁺=451.05.

Compound 295: ¹H-NMR (400 MHz, cdcl₃) δ 8.25 (s, 1H), 7.97 (s, 1H), 7.86 (s, 1H), 7.75 (d, J=3.2 Hz, 1H), 3.66 (s, 2H), 2.85-2.67 (m, 3H), 2.15 (ddd, J=24.0, 14.3, 9.6 Hz, 1H), 2.01 (dd, J=12.0, 5.2 Hz, 2H), 1.35 (d, J=2.0 Hz, 6H), 1.24-1.09 (m, 2H), 0.89 (dt, J=6.4, 4.8 Hz, 2H). MS [M+H]⁺=435.03.

Compound 296: ¹H-NMR (400 MHz, DMSO-d6) δ 8.18 (t, J=5.3 Hz, 1H), 8.09 (d, J=0.9 Hz, 1H), 8.00 (d, J=1.8 Hz, 1H), 7.82 (d, J=1.7 Hz, 1H), 7.02 (t, J=5.9 Hz, 1H), 3.02 (dd, J=12.9, 6.7 Hz, 2H), 2.88 (d, J=7.9 Hz, 3H), 2.75 (t, J=3.2 Hz, 3H), 2.32-2.20 (m, 1H), 1.78 (p, J=6.9 Hz, 2H), 1.09 (ddd, J=8.3, 6.6, 4.3 Hz, 2H), 0.92 (dt, J=6.8, 4.5 Hz, 2H). MS [M+H]⁺=486.13.

Compound 297: ¹H-NMR (400 MHz, cdcl₃) δ 7.99 (s, 1H), 7.88 (d, J=1.7 Hz, 1H), 7.77 (s, 1H), 4.14 (dd, J=7.7, 6.3 Hz, 4H), 3.98 (dd, J=4.4, 2.3 Hz, 2H), 3.94 (dd, J=7.7, 6.2 Hz, 2H), 3.89 (dd, J=4.5, 2.3 Hz, 2H), 3.68 (s, 2H), 2.88-2.70 (m, 2H), 2.24-2.09 (m, 2H), 1.27-1.06 (m, 2H), 0.97-0.80 (m, 2H). MS [M+H]⁺=437.11.

Compound 298: ¹H-NMR (400 MHz, DMSO-d6) δ 8.24 (t, J=5.4 Hz, 1H), 8.09 (s, 1H), 8.00 (s, 1H), 7.83 (s, 1H), 3.94 (s, 2H), 3.61 (s, 2H), 3.19 (d, J=7.5 Hz, 2H), 3.05 (s, 2H), 2.75 (s, 3H), 2.33-2.19 (m, 1H), 2.07-1.91 (m, 2H), 1.14-1.02 (m, 2H), 0.98-0.86 (m, 2H). MS [M+H]⁺=478.19.

Compound 299: ¹H-NMR (400 MHz, DMSO-d6) δ 8.28 (d, J=5.9 Hz, 1H), 8.09 (s, 1H), 8.02 (s, 1H), 7.85 (s, 1H), 3.97 (d, J=9.2 Hz, 2H), 3.86 (d, J=12.3 Hz, 2H), 3.62 (s, 3H), 3.53 (d, J=9.6 Hz, 2H), 3.24 (s, 1H), 2.76 (s, 2H), 2.33-2.21 (m, 2H), 1.17-1.02 (m, 2H), 0.93 (dd, J=7.2, 4.1 Hz, 2H). MS [M+H]⁺=450.25.

Compound 300: ¹H-NMR (400 MHz, DMSO-d6) δ 8.09 (s, 1H), 8.00 (s, 1H), 7.83 (s, 1H), 7.73 (s, 2H), 3.64 (t, J=6.9 Hz, 2H), 2.85 (dd, J=13.7, 6.6 Hz, 2H), 2.79 (d, J=27.7 Hz, 3H), 2.31-2.20 (m, 1H), 2.00-1.83 (m, 2H), 1.14-1.03 (m, 2H), 0.98-0.83 (m, 2H). MS [M+H]⁺=422.17.

Compound 301: ¹H-NMR (400 MHz, DMSO-d6) δ 8.18 (s, 1H), 8.09 (s, 1H), 8.00 (s, 1H), 7.83 (s, 1H), 3.67 (d, J=5.7 Hz, 1H), 3.51 (d, J=13.1 Hz, 1H), 3.44-3.14 (m, 3H), 2.75 (s, 3H), 2.47 (s, 4H), 2.47-1.90 (m, 9H), 1.08 (d, J=6.3 Hz, 2H), 0.99-0.78 (m, 2H). MS [M+H]⁺=425.20.

Compound 302: ¹H-NMR (400 MHz, cdcl₃) δ 7.90 (s, 1H), 7.77 (s, 1H), 7.68 (s, 1H), 7.19 (s, 2H), 4.06 (s, 2H), 3.70 (d, J=5.7 Hz, 1H), 3.62-3.37 (m, 1H), 2.68 (s, 3H), 1.24 (t, J=6.5 Hz, 3H), 1.19 (d, J=8.3 Hz, 2H), 1.16-1.05 (m, 3H), 0.88-0.72 (m, 4H). MS [M+H]⁺=423.18.

Compound 303: ¹H-NMR (400 MHz, DMSO-d6) δ 8.17 (s, 1H), 8.09 (s, 1H), 8.00 (s, 1H), 7.83 (s, 1H), 3.67 (d, J=6.1 Hz, 1H), 3.51 (d, J=13.3 Hz, 1H), 3.34 (dd, J=11.7, 5.5 Hz, 2H), 3.29-3.18 (m, 1H), 2.75 (s, 3H), 2.25 (d, J=5.0 Hz, 1H), 1.08 (d, J=6.3 Hz, 2H), 0.92 (d, J=4.6 Hz, 2H). MS [M+H]⁺=425.22.

Compound 304: ¹H-NMR (400 MHz, DMSO-d6) δ 8.08 (s, 1H), 8.04 (s, 1H), 7.99 (s, 1H), 7.82 (s, 1H), 3.25 (d, J=5.9 Hz, 2H), 3.16 (s, 3H), 2.74 (s, 2H), 2.27 (d, J=13.7 Hz, 2H), 1.08 (d, J=6.4 Hz, 2H), 0.92 (d, J=6.0 Hz, 3H), 0.85 (s, 6H). MS [M+H]⁺=437.28.

### Preparation of Compound 305

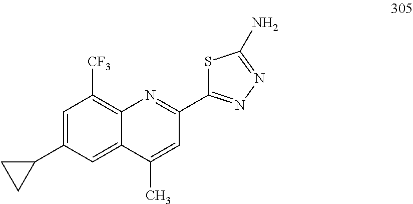

To 6-Cyclopropyl-4-methyl-8-trifluoromethyl-quinoline-2-carbonitrile, the preparation of which is described elsewhere in procedures for example compound 286, 40 mg (0.14 mmol), dissolved in 2 mL TFA was added 15 mg (1.1 equiv) thiosemicarbazide. The reaction was heated at 65° C. for 1 h, at which time the solvent was removed by co-evaporation with toluene and the residue purified by reverse phase HPLC to give 12 mg final product. ¹H-NMR (400 MHz, DMSO-d6) δ 8.09 (s, 1H), 8.00 (s, 1H), 7.83 (s, 1H), 7.59 (m, 1H), 2.74 (s, 3H), 2.26 (m, 1H), 1.09 (d, 2H), 0.92 (d, 2H). MS [M+H]⁺=351.

### Example 33

### Preparation of Compounds 306-309

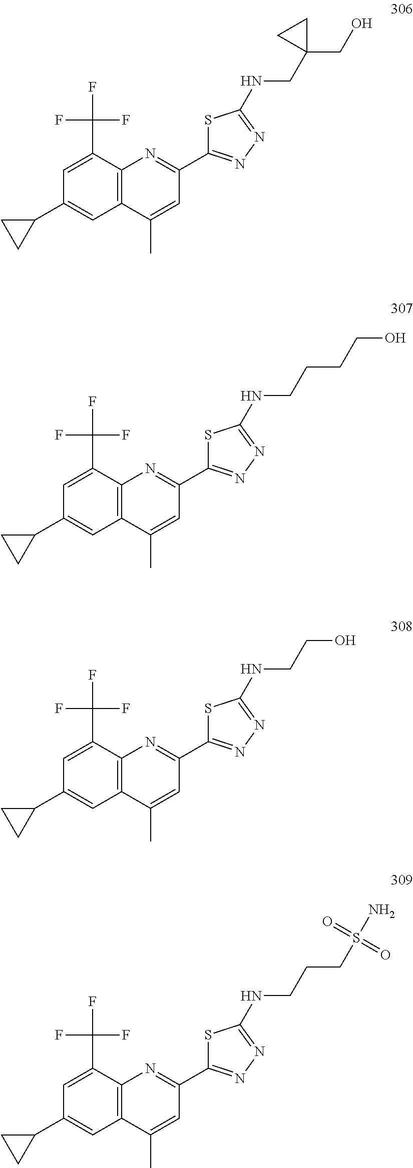

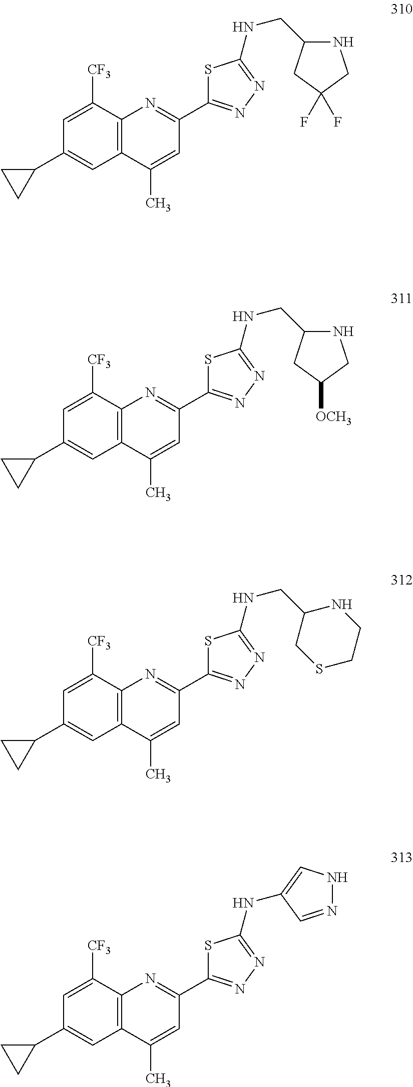

The compounds in the example were made according to the procedure for example compound 286.

Compound 306: ¹H-NMR (400 MHz, DMSO) δ 8.17 (s, 1H), 8.12 (s, 1H), 8.03 (s, 1H), 7.86 (s, 1H), 3.41 (d, J=5.5 Hz, 2H), 3.34 (s, 1H), 2.78 (s, 3H), 2.29 (dd, J=13.6, 8.6 Hz, 1H), 1.16-1.06 (m, 2H), 0.96 (q, J=4.6 Hz, 2H), 0.49 (d, J=3.6 Hz, 2H), 0.44 (d, J=3.6 Hz, 2H). MS [M+H]⁺=435.

Compound 307: ¹H-NMR (400 MHz, DMSO) δ 8.15 (s, 1H), 8.08 (s, 1H), 7.99 (s, 1H), 7.82 (s, 1H), 3.40 (t, J=6.4 Hz, 2H), 3.33 (dd, J=12.5, 6.9 Hz, 2H), 2.75 (s, 3H), 2.27 (d, J=13.9 Hz, 1H), 1.60 (dd, J=14.9, 7.1 Hz, 2H), 1.48 (dd, J=14.7, 6.3 Hz, 2H), 1.13-1.00 (m, 2H), 0.97-0.84 (m, 2H). MS [M+H]⁺=423.

Compound 308: ¹H-NMR (400 MHz, DMSO) δ 8.21 (s, 1H), 8.08 (s, 1H), 7.99 (s, 1H), 7.82 (s, 1H), 3.57 (t, J=5.7 Hz, 2H), 3.41 (q, J=5.4 Hz, 2H), 2.75 (s, 3H), 2.32-2.15 (m, 1H), 1.18-1.02 (m, 2H), 0.93 (dd, J=8.0, 3.1 Hz, 2H). MS [M+H]⁺=395.

Compound 309: ¹H-NMR (400 MHz, DMSO) δ 8.22 (s, 1H), 8.09 (s, 1H), 8.00 (s, 1H), 7.83 (s, 1H), 6.80 (s, 2H), 3.47 (d, J=5.9 Hz, 2H), 3.13-2.92 (m, 2H), 2.72 (d, J=22.0 Hz, 3H), 2.26 (s, 1H), 2.09-1.98 (m, 2H), 1.13-0.97 (m, 2H), 0.92 (d, J=6.7 Hz, 2H). MS [M+H]⁺=472.

Compound 310: 21% yield after HPLC purification. ¹H-NMR (400 MHz, CDCl₃) δ 8.80 (s, 1H), 8.08 (s, 1H), 7.89 (s, 1H), 7.48 (s, 1H), 4.24 (d, 2H), 1.13-1.00 (m, 2H), 0.97-0.84 (m, 2H). MS [M+H]⁺=470.

Compound 311: 32% yield after HPLC purification. ¹H-NMR (400 MHz, CDCl₃) diagnostic peaks at δ 9.10 (s, 1H), 8.12 (s, 1H), 7.94 (s, 1H), 7.89 (s, 1H), 7.55 (s; 1H), 2.75 (s, 3H), 1.12-1.00 (m, 2H), 0.98-0.84 (m, 2H). MS [M+H]⁺=464.

Compound 312: 27% yield after HPLC purification. ¹H-NMR (400 MHz, CDCl₃) δ 8.16 (s, 1H), 8.10 (s, 1H), 7.84 (s, 1H), 7.49 (s, 1H), 4.25 (d, 2H), 2.25 (m, 1H), 1.13-1.00 (m, 2H), 0.97-0.84 (m, 2H). MS [M+H]⁺=466.

Compound 313: 15% yield after HPLC purification. ¹H-NMR (400 MHz, CDCl₃) δ 10.3 (s, 1H), 8.13 (s, 1H), 8.01 (s, 1H), 7.83 (s, 2H), 2.77 (s, 3H), 2.27 (m, 1H), 1.13-1.00 (m, 2H), 0.97-0.84 (m, 2H). MS [M+H]⁺=417.

### Example 34

### Compounds 314-320

### Preparation of 314

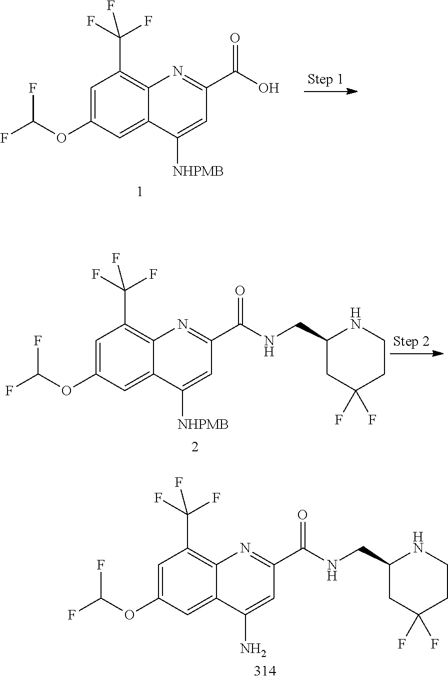

Compound 314 (16.2 mg, 67%) was prepared from compound 1 in a manner similar to that described previously.

¹H-NMR (400 MHz, CD₃OD) δ 8.14 (d, J=2.4 Hz, 1H), 7.87 (d, J=2.4 Hz, 1H), 7.46 (s, 1H), 7.00 (t, J=73.2 Hz, 1H), 3.82 (dd, J=14.4 and 6.8 Hz, 1H), 3.76 (dd, J=14.4 and 4.0 Hz, 1H), 3.65-3.73 (m, 1H), 3.55 (dm, J=13.2 Hz, 1H), 3.19 (td, J=13.6 and 3.2 Hz, 1H), 2.50 (m, 1H), 2.04-2.41 (m, 3H); ¹⁹F NMR (376.1 MHz, CDCl₃) δ −62.21 (s, 3F), −77.74 (s, 6F), −84.72 (d, J=72.58 Hz, 2F), −95.41 (d, J=245.2 Hz, 1F), −103.39 (dtt, J=245.2, 32.2, and 10.3 Hz, 1F); MS [M+H]⁺=455.1

### Preparation of Compounds 315

Compound 315 was prepared in manners similar to compound 314

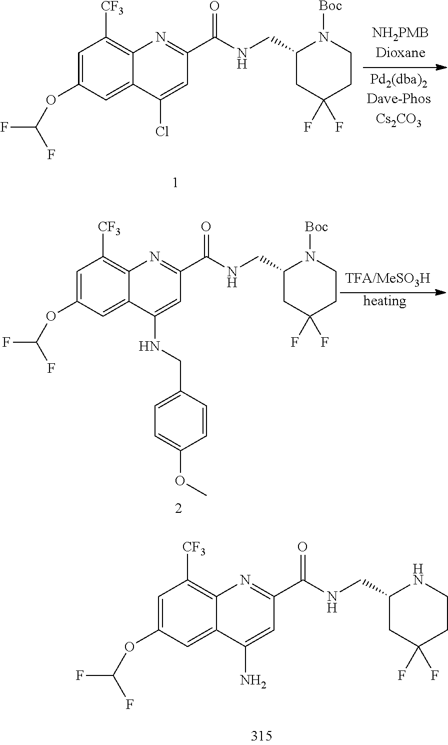

¹H-NMR for 315 (400 MHz, CDCl₃) δ 7.76 (m, 2H), 7.35 (m, 1H), 6.64 (t, 1H), 3.46 (m, 2H), 3.06 (m, 2H), 2.80 (m, 2H), 2.07-1.50 (m, 4H); ¹⁹F NMR (376.1 MHz) δ −61.25 (s, 3F), 82.5 (d, 2F), 89.0 (d, 1F), 101.5-102.1 (m, 1F); MS [M+H]⁺=455.0.

### Preparation of 316

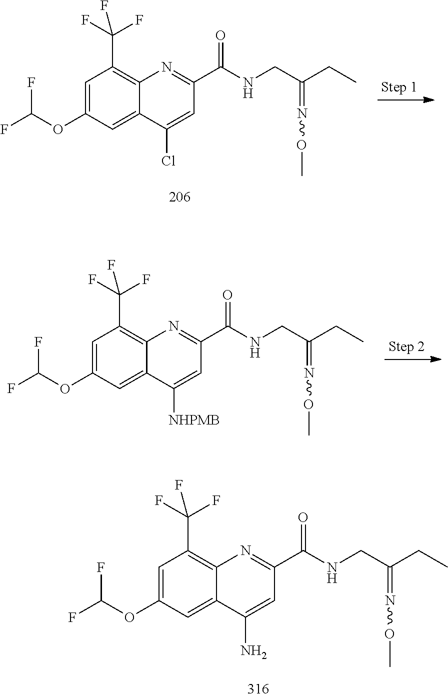

Compound 316 (25.3 mg, 14%) was prepared from compound 206 in a manner similar to that described previously.

¹H-NMR (400 MHz, CD₃OD) δ 8.13 (d, J=2.4 Hz, 1H), 7.87 (d, J=2.4 Hz, 1H), 7.43 (s, 0.6H), 7.41 (s, 0.4H), 7.01 (t, J=73.2 Hz, 1H), 4.30 (s, 0.8H), 4.17 (s, 1.2H), 3.90 (s, 1.2H), 3.89 (s, 1.8H), 2.38 (q, J=7.6 Hz, 1.2H), 2.28 (q, J=7.6 Hz, 0.8H), 1.11 (t, J=7.6 Hz, 3H); ¹⁹F NMR (376.1 MHz, CDCl₃) δ −62.27 (s, 1.8F), −62.43 (s, 1.2F), −78.14 (s, 3F), −84.72 (d, J=72.59 Hz, 1.2F), −84.74 (d, J=73.34 Hz, 0.8); MS [M+H]⁺=421.0

### Compound 317

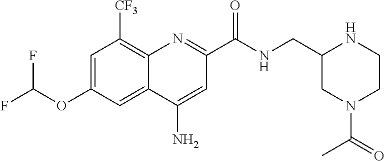

¹H-NMR (400 MHz, CD3OD) δ 8.14 (s, 1H), 7.87 (s, 1H), 7.45 (s, 1H), 4.64-4.51 (dd, 1H), 4.19-4.02 (dd, 1H), 3.85-3.69 (m, 2H), 3.59-3.34 (m, 3H), 3.29-2.96 (m, 3H), 2.14 (s, 3H); ¹⁹F NMR (376.1 MHz) δ −62.25 (s), −84.85 (d); MS [M−H]⁺=462.2.

### Compound 318

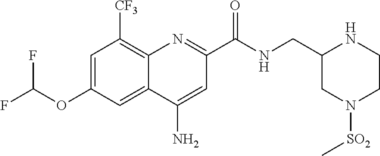

¹H-NMR (400 MHz, CD3OD) δ 8.12 (d, 1H), 7.86 (d, 1H), 7.45 (s, 1H), 7.00 (s, 1H), 3.89-3.71 (m, 3H), 3.66 (m, 1H), 3.53 (m, 1H), 3.29 (m, 1H), 3.21 (m, 3H), 3.09 (m, 1H), 2.95 (s, 3H); ¹⁹F NMR (376.1 MHz) δ −62.22 (s), −84.81 (d); MS [M−H]⁺=498.1.

### Example 35

Compounds 319-337 were prepared in manners similar to compound 230

### Compound 319

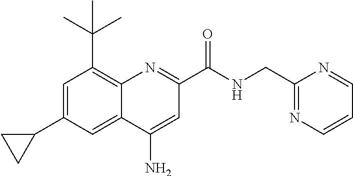

¹H-NMR (400 MHz, CDCl3) δ 8.79 (m, 2H), 8.18 (s, 1H), 7.64 (s, 1H), 7.65 (s, 1H), 7.28 (m, 1H), 4.99 (d, 2H), 2.09 (m, 1H), 1.64 (s, 9H), 1.16 (m, 2H), 0.91 (m, 2H); MS [M−H]⁺=376.26.

### Compound 320

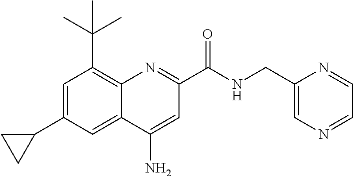

¹H-NMR (400 MHz, CDCl3) δ 8.63 (s, 1H), 8.49 (s, 1H), 8.43 (d, 1H), 8.17 (s, 1H), 7.67 (s, 1H), 7.54 (d, 2H), 4.82 (d, 2H), 1.99 (m, 1H), 1.53 (s, 9H), 1.04 (m, 2H), 0.81 (m, 2H); MS [M−H]⁺=376.26.

### Compound 321

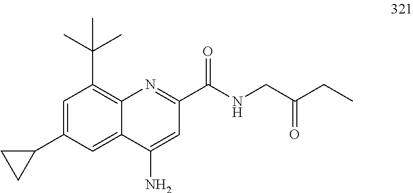

¹H-NMR (400 MHz, CDCl3) δ 8.63 (s, 1H), 8.49 (s, 1H), 8.43 (d, 1H), 8.17 (s, 1H), 7.67 (s, 1H), 7.54 (d, 2H), 4.82 (d, 2H), 1.99 (m, 1H), 1.53 (s, 9H), 1.04 (m, 2H), 0.81 (m, 2H); MS [M−H]⁺=376.26.

### Compound 322

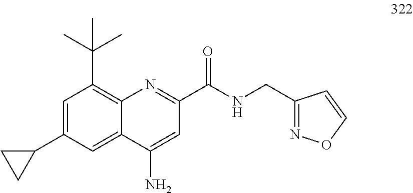

¹H-NMR (400 MHz, CDCl₃) δ 8.54 (br, 1H), 8.35 (m, 1H), 7.49 (m, 1H), 7.37 (m, 1H), 7.29 (s, 1H), 6.40 (m, 1H), 4.82 (m, 4H), 2.05 (m, 1H), 1.61 (s, 9H), 1.03 (m, 2H), 0.80 (m, 2H); MS [M+H]⁺=365.2.

### Compound 323

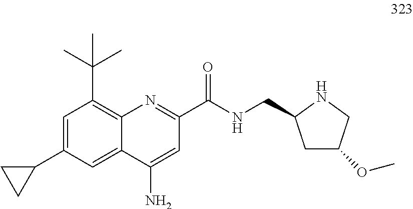

¹H-NMR (400 MHz, CH₃OH-d₄) δ 7.90 (s, 1H), 7.80 (s, 1H), 7.46 (s, 1H), 4.18 (m, 1H), 4.06 (m, 1H), 3.82 (m, 2H), 3.56 (m, 1H), 3.40 (m, 1H), 3.34 (s, 3H), 2.42 (m, 1H), 2.18 (m, 1H), 1.96 (m, 1H), 1.62 (s, 9H), 1.18 (m, 2H), 0.93 (m, 2H); MS [M+H]⁺=397.

### Compound 324

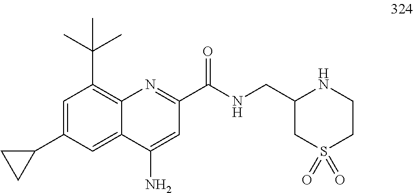

¹H-NMR (400 MHz, CH₃OH-d₄) δ 7.88 (m, 2H), 7.38 (s, 1H), 3.92-3.30 (m, 9H), 2.18 (m, 1H), 1.62 (s, 9H), 1.18 (m, 2H), 0.93 (m, 2H); MS [M+H]⁺=431.

### Compound 325

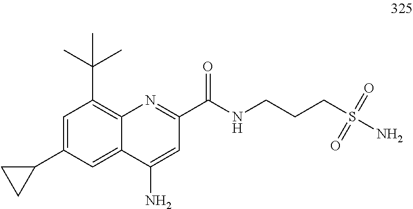

¹H-NMR (400 MHz, CH₃OH-d₄) δ 7.87 (s, 1H), 7.80 (s, 1H), 7.34 (s, 1H), 3.66 (m, 2H), 3.20 (m, 2H), 2.20 (m, 3H), 1.63 (s, 9H), 1.15 (m, 2H), 0.91 (m, 2H); MS [M+H]⁺=405.

### Compound 326

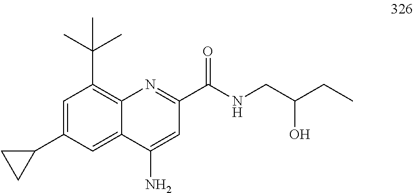

¹H-NMR (400 MHz, CH₃OH-d₄) δ 7.89 (s, 1H), 7.82 (s, 1H), 7.36 (s, 1H), 3.85 (m, 2H), 3.20 (m, 1H), 2.20 (m, 1H), 1.63 (s, 9H), 1.44 (m, 2H), 1.24 (m, 3H), 1.12 (m, 2H), 0.93 (m, 2H); MS [M+H]⁺=356.

### Compound 327

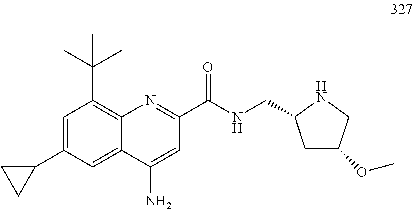

¹H-NMR (400 MHz, CH₃OH-d₄) δ 7.90 (s, 1H), 7.82 (s, 1H), 7.46 (s, 1H), 4.18 (m, 1H), 4.06 (m, 1H), 3.82 (m, 2H), 3.56 (m, 1H), 3.38 (m, 1H), 3.34 (s, 3H), 2.42 (m, 1H), 2.18 (m, 1H), 2.02 (m, 1H), 1.62 (s, 9H), 1.18 (m, 2H), 0.93 (m, 2H); MS [M+H]⁺=397.

### Compound 328

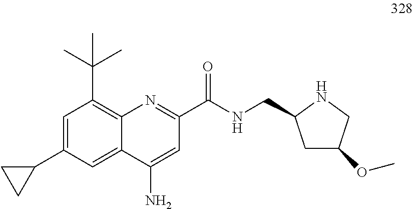

¹H-NMR (400 MHz, CH₃OH-d₄) δ 7.90 (s, 1H), 7.82 (s, 1H), 7.47 (s, 1H), 4.18 (m, 1H), 4.06 (m, 1H), 3.82 (m, 2H), 3.58 (m, 1H), 3.40 (m, 1H), 3.34 (s, 3H), 2.42 (m, 1H), 2.18 (m, 1H), 2.02 (m, 1H), 1.62 (s, 9H), 1.18 (m, 2H), 0.93 (m, 2H); MS [M+H]⁺=397.

### Compound 329

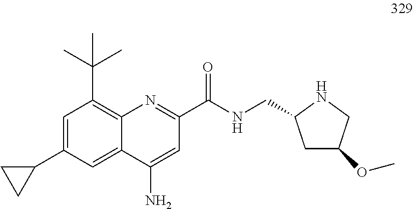

¹H-NMR (400 MHz, CH₃OH-d₄) δ 7.90 (s, 1H), 7.82 (s, 1H), 7.47 (s, 1H), 4.18 (m, 1H), 4.06 (m, 1H), 3.85 (m, 2H), 3.58 (m, 1H), 3.40 (m, 1H), 3.36 (s, 3H), 2.42 (m, 1H), 2.18 (m, 1H), 1.96 (m, 1H), 1.62 (s, 9H), 1.18 (m, 2H), 0.93 (m, 2H); MS [M+H]⁺=397.

### Compound 330

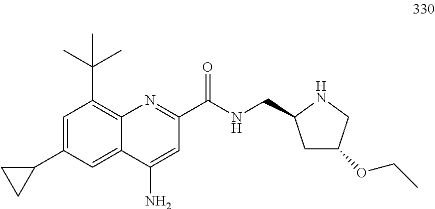

¹H-NMR (400 MHz, CH₃OH-d₄) δ 7.90 (s, 1H), 7.80 (s, 1H), 7.47 (s, 1H), 4.25 (m, 1H), 4.06 (m, 1H), 3.84 (m, 2H), 3.56 (m, 3H), 3.40 (m, 1H), 2.42 (m, 1H), 2.18 (m, 1H), 1.96 (m, 1H), 1.62 (s, 9H), 1.18 (m, 5H), 0.93 (m, 2H); MS [M+H]⁺=411.

### Compound 331

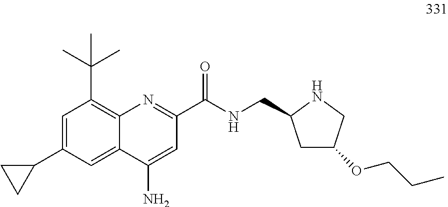

¹H-NMR (400 MHz, CH₃OH-d₄) δ 7.90 (s, 1H), 7.80 (s, 1H), 7.47 (s, 1H), 4.25 (m, 1H), 4.06 (m, 1H), 3.84 (m, 2H), 3.56 (m, 1H), 3.40 (m, 3H), 2.42 (m, 1H), 2.18 (m, 1H), 1.96 (m, 1H), 1.62 (s, 9H), 1.58 (m, 2H), 1.18 (m, 2H), 0.93 (m, 5H); MS [M+H]⁺=425.

### Compound 332

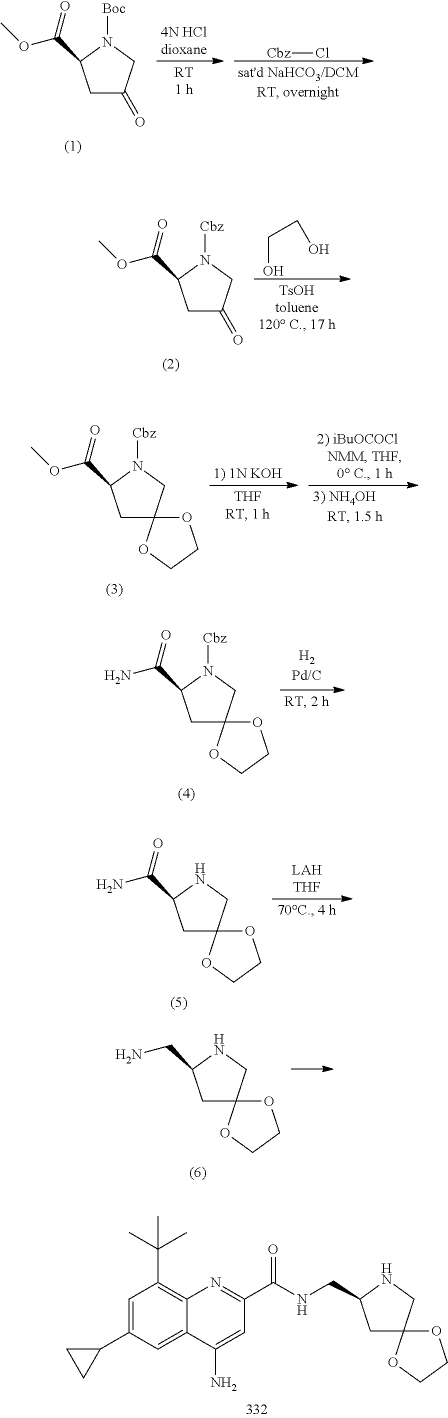

(S)-1-tert-butyl 2-methyl 4-oxopyrrolidine-1,2-dicarboxylate (1) (7.0 g, 28.8 mmol) was dissolved in DCM (30 mL), then added 4N HCl in dioxane (30 mL). The reaction mixture was stirred at RT for 1 h. After the reaction completed, it was concentrated to dryness. To the residue, it was dissolved in EtOAc (200 mL) and sat'd NaHCO₃ (200 mL). Cbz-Cl (16.3 mL, 115 mmol) was added. The reaction mixture was stirred at RT overnight. The layers were separated. The organic layer was concentrated and purified by flash chromatography on silica gel with EtOAc/Hexane to give 6.0 g (75%) of (2).

Compound (2) (5.58 g, 20.1 mmol) in 100 mL toluene with ethylene glycol (21.7 mL, 388.8 mmol) and p-TsOH (534 mg, 2.8 mmol) was placed in a flask equipped with a Dean-Stark Trap. It was heated to 120° C. for 17 h. Cooled to RT, then concentrated by vacuum, the residue was dissolved in EtOAc, washed with sat'd NaHCO₃ and brine. The organic phase was dried (MgSO₄) and concentrated to give a crude mixture which was purified by flash chromatography on silica gel with EtOAc/Hexane to give 3.55 g (55%) of (3).

Compound (3) (3.55 g, 11.0 mmol) in 44 mL THF/4.4 mL MeOH and 22 mL 1M KOH was stirred at RT for 1 h. After the completion of the reaction, it was acidified with 1N HCl to pH<4, extracted with EtOAc twice. The organic phase was dried (MgSO₄) and concentrated to give acid of (3). The acid was dissolved in THF (40 mL) with NMM (3.63 mL, 33 mmol). It was cooled to 0° C. under N₂. Isobutyl-chloroformate (1.615 mL, 12.1 mmol) was added dropwise over 5 min and the mixture was stirred for 60 min @ 0° C. It was then added NH₄OH (7.43 mL, 110 mmol). After stirred at 0° C. for 15 min, RT for 90 min, the reaction was done. It was extracted with EtOAc twice. The organic phase was washed with brine and dried (Na₂SO₄) and concentrated to give a crude mixture which was purified by flash chromatography on silica gel with EtOAc/Hexane to give 1.71 g (51%) of (4).

Compound (4) (0.64 g, 2.09 mmol) was deprotected to (5) by 10% Pd/C hydrogenation in EtOAc/EtOH. After removal of catalyst and solvent, it was dissolved in THF (10 mL) and added LAH at RT. It was stirred until bubble ceased then heated to 70° C. for 3 h. After completion of the reaction, it was cooled to 0° C., added 0.5 mL of water, 0.5 mL of 15% NaOH and another 0.5 mL of water sequentially. It was then diluted with Ether (100 mL), stirred for 30 min at 0° C. before filtering. The filtrate was concentrated. The residue was added EtOAc, dried (Na₂SO₄) and concentrated to give 246 mg (74%) of (6).

Compound 322 ¹H-NMR (400 MHz, CDCl₃) δ 8.49 (b, 1H), 7.46 (m, 1H), 7.36 (m, 1H), 7.32 (m, 1H), 4.85 (s, 2H), 3.89 (m, 4H), 3.56 (m, 3H), 3.00 (m, 2H), 2.13-1.76 (m, 3H), 1.63 (d, 9H), 1.02 (m, 2H), 0.79 (m, 2H); MS [M+H]⁺=425.2

### Compound 333

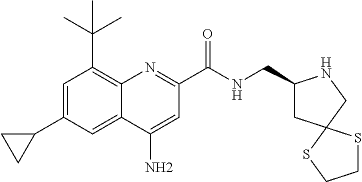

¹H-NMR (400 MHz, CD₃OD₃) δ 7.57 (m, 1H), 7.39 (m, 1H), 7.27 (s, 1H), 3.59-3.54 (m, 3H), 3.35-3.14 (m, 6H), 2.46 (m, 1H), 2.17 1.99 (m, 2H), 1.65 (s, 9H), 1.01 (m, 2H), 0.82 (m, 2H); MS [M+H]⁺=457.2

### Compound 334

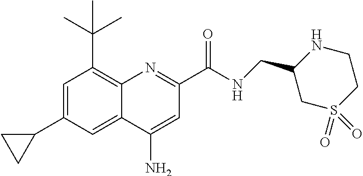

¹H-NMR (400 MHz, DMSO-d6) δ 7.88 (s, 1H), 7.81 (s, 1H), 7.43 (s, 1H), 5.47 (s, 2H), 4.1-3.2 (m, 10H), 2.1 (m, 1H), 1.6 (s, 9H), 1.26 (m, 2H), 0.89 (m, 2H).

MS [M+H]⁺=431.25.

### Compound 335

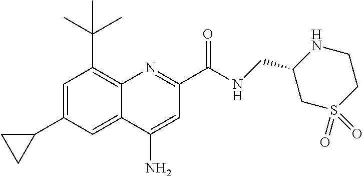

¹H-NMR (400 MHz, DMSO-d6) δ 7.88 (s, 1H), 7.81 (s, 1H), 7.43 (s, 1H), 5.47 (s, 2H), 4.1-3.2 (m, 10H), 2.1 (m, 1H), 1.6 (s, 9H), 1.26 (m, 2H), 0.89 (m, 2H).

MS [M+H]⁺=431.27

### Compound 336

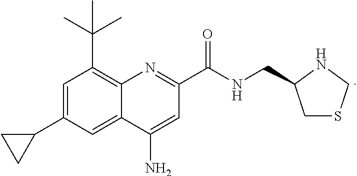

¹H-NMR (400 MHz, DMSO-d6) δ 7.9 (s, 1H), 7.88 (s, 1H), 7.82 (s, 1H), 7.39 (s, 1H), 4.45 (dd, 2H), 4.13 (m, 1H), 3.88 (m, 2H), 3.38 (m, 1H), 3.09 (dd, 1H), 2.16 (m, 1H), 1.64 (s, 9H), 1.15 (m, 2H), 0.9 (m, 2H).

MS [M+H]⁺=385.19

### Compound 337

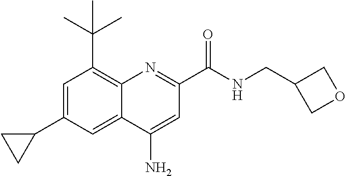

¹H-NMR (400 MHz, DMSO-d6) δ 7.58 (d, 1H), 7.4 (d, 1H), 7.27 (s, 1H), 4.81 (m, 2H), 4.53 (t, 2H), 3.75 (d, 2H), 2.04 (m, 1H), 1.63 (s, 9H), 1.02 (m, 2H), 0.81 (m, 2H).

MS [M+H]⁺=354.23

### Example 36

### Compound 338

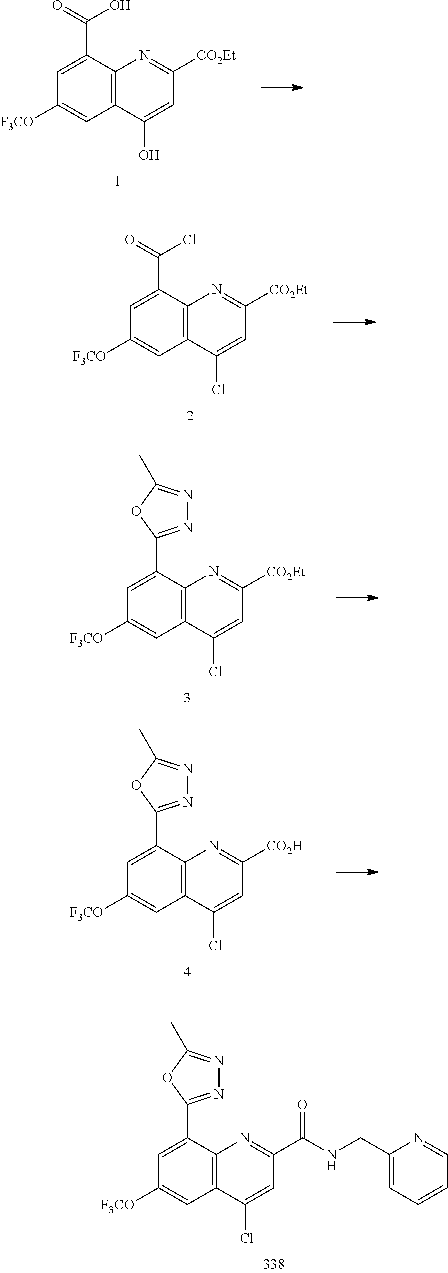

Compound 1 was obtained from the standard condensation chemistry reported elsewhere in these procedures, utilizing diethyl acetylene dixcarboxylate and 2-carboxylate, 4-trifluoromethoxy aniline starting material. Following a series of standard transformations, the method reported in the Journal of Heterocyclic Chemistry (1984), 21(6), p. 1807-1816 was used. Following this procedure, acetyl hydrazide is a reacting partner, and is used to install the methyl oxadiazole of 3. Subsequent ester hydrolysis and amide formation using common methods gave 338 in moderate yield.

Compound 3384: Obtained in 15% yield after HPLC purification. ¹H-NMR (400 MHz, CDCl₃) diagnostic peaks at δ 8.06 (s, 1H), 6.40 (s, 1H), 6.17 (s, 1H), 3.76 (s, 3H), 3.67 (s, 2H). MS [M+H]⁺=464.

### Compound 339

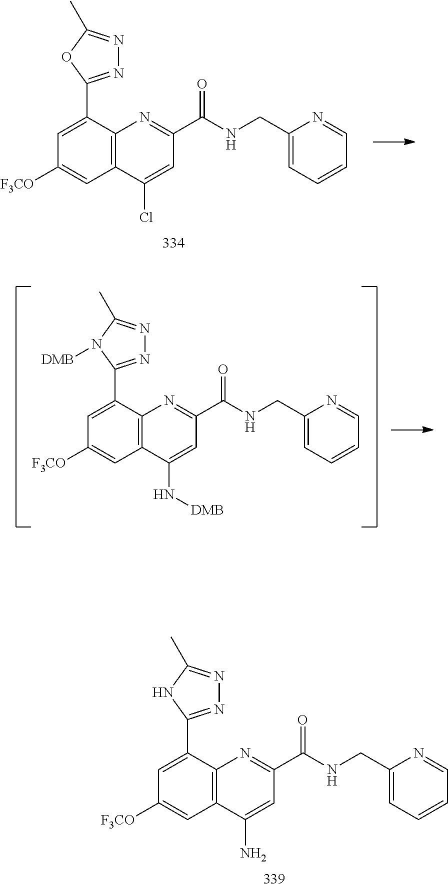

Treatment of 338 with excess bis-DMB amine at 130° C. followed by TFA resulted in 339, which was purified by HPLC chromatography. ¹H-NMR (400 MHz, CDCl₃) diagnostic peaks at δ 9.29 (s, 2H), 8.72 (s, 1H), 8.15 (s, 1H), 8.08 (s, 1H), 7.99 (s, 1H), 7.82 (s, 1H), 5.11 (d, 2H), 2.73 (s, 3H), MS [M+H]⁺=444.

### Example 37

### Compound 340

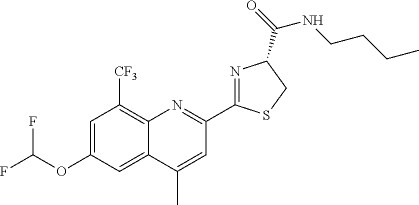

### Compound 339 was prepared from 186

¹H-NMR (400 MHz, DMSO-d6) δ 8.28 (s, 1H), 8.06 (m, !H), 8.02 (d, 1H), 7.94 (d, 1H), 7.14 (t, 1H), 5.31 (t, 1H), 3.66 (m, 1H), 3.49 (m, 1H), 2.79 (s, 3H), 1.54 (m, 2H), 1.36 (m, 2H), 1.16 (m, 2H), 0.93 (t, 3H).

¹⁹F NMR (376.1 MHz) δ −62.26 (s), −85.28 (d).

MS [M+H]⁺=462.2.

### Compound 341

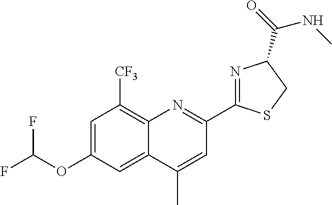

Compound 341 was prepared similarly to 340.

¹H-NMR (400 MHz, DMSO-d6) δ 8.22 (s, 1H), 8.06 (m, !H), 7.94 (d, 1H), 7.86 (d, 1H), 7.07 (t, 1H), 5.24 (t, 1H), 3.59 (m, 1H), 3.43 (m, 1H), 2.75 (d, 3H), 2.71 (s, 3H).

### Example 38

### Preparation of Compound 342-361

### Compound 342

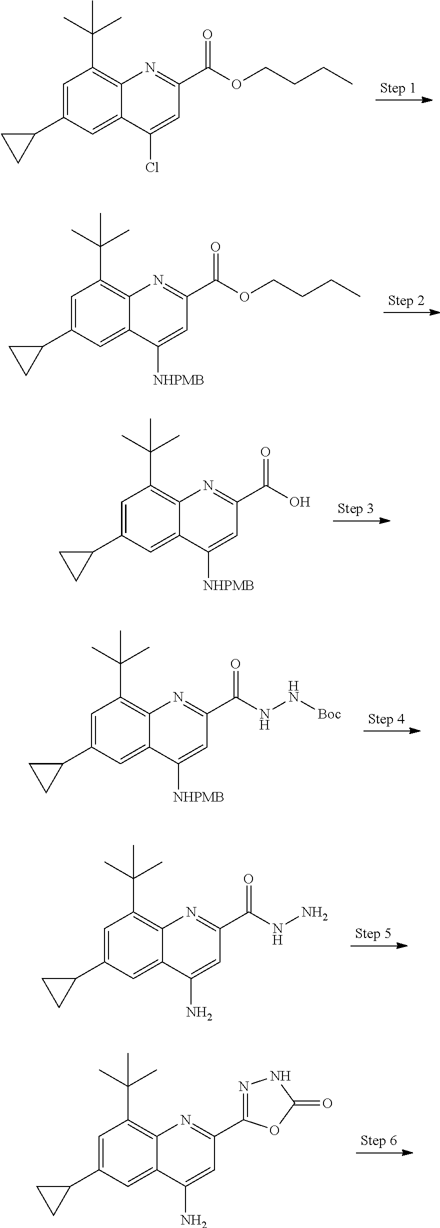

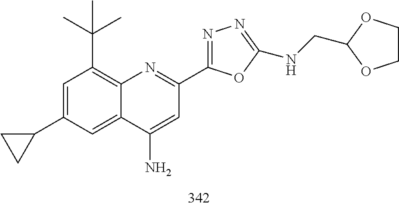

### Step 1

A 1-L 3 neck rbf was charged with Pd₂(dba)₃ (672 mg, 0.175 mmol), DavePhos® 1.18 g, 3.01 mmol) and cesium carbonate (29.3 g, 90.3 mmol). The reaction flask was evacuated and back-filled with N₂ (3×) and the solids taken up in 250 mL dioxane. After 5 min stirring, a solution of compound K (10.8 g, 30.1 mmol) in degassed dioxane was added, followed by p-methoxybenzylamine (5.8 mL, 45 mmol). The reaction mixture was heated at 100° C. overnight. Aqueous work-up (EtOAc, water) and silica gel chromatography provided compound O (11.1 g, 83% yield) as a tan solid. MS [M+H]⁺=461.19.

### Step 2

Compound O was hydrolyzed using the same procedure in Example 1, step 6 to provide Compound P. [M+H]⁺=405.32.

### Step 3

A solution of compound P (3.135 g, 7.75 mmol) in 60 mL DMF was treated with tert-butyl carbazate (1.53 g, 11.6 mmol), NMM (3.7 mL, 34.8 mmol) and BOP (5.14 g, 11.6 mmol). After 5 min the reaction was diluted with 350 mL EtOAc and washed with 5% LiCl solution, 10% citric acid, sat. NaHCO₃ and brine. The organic layer was dried with sodium sulfate, concentrated in vacuo and the residue purified by ISCO chromatography to provide the desired intermediate 3 (2.40 g, 59% yield) as a white solid. [M+H]⁺=519.16

### Step 4

Intermediate 3 (2.4 g, 4.57 mmol) was suspended in 16 mL DCM and treated with 16 mL TFA. After 30 min the homogeneous solution was diluted with EtOAc and aq. potassium carbonate. The organic layer was concentrated to provide intermediate 4 (1.73 g, 69% yield) as a yellow solid.

MS [M+H]⁺=299.18.

### Step 5

A solution of intermediate 4 (91 mg, 0.33 mmol) and DIEA (172 uL, 1.0 mmol) in 10 mL DCM was treated with a solution of triphosgene (59 mg, 0.198 mmol) in 1 mL DCM. After stirring for 2 h at rt, 50 mL water was added and the reaction stirred vigorously until only the free C-4 amino product was visible by LCMS. The organic layer was separated, dried with sodium sulfate and concentrated in vacuo to provide the desired product (103 mg, 96% yield) as a white solid. MS [M+H]⁺=325.22.

### Step 6

A solution of substrate (65 mg, 0.20 mmol) in 1.5 mL DMF was treated with (1,3-dioxolan-2-yl)methanamine (41 mg, 0.40 mmol), DIEA (68 uL, 0.40 mmol) and BOP (97 mg, 0.22 mmol) and allowed to stir for 2 h at rt. The reaction was diluted with EtOAc and washed with 10% citric acid, sat. NaHCO₃ and brine. Silica gel chromatography provided the desired product 342 (32 mg, 39% Yield) as a tan powder; ¹H-NMR (400 MHz, DMSO) δ 8.04 (bs, 1H), 7.58 (s, 1H), 7.34 (s, 1H), 7.11 (s, 1H), 5.03 (t, J=4 Hz, 1H), 3.90 (m, 2H), 3.79 (m, 2H), 3.60 (t, J=5 Hz, 2H), 2.01 (m, 1H), 0.99 (m, 2H), 0.81 (m, 2H); MS [M−H]⁺=410.34.

### Compound 343

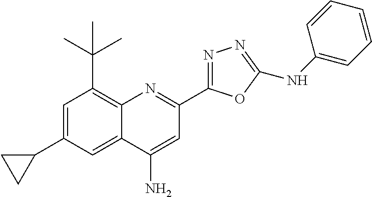

¹H-NMR (400 MHz, DMSO) δ 8.04 (bs, 1H), 7.58 (s, 1H), 7.34 (s, 1H), 7.11 (s, 1H), 5.03 (t, J=4 Hz, 1H), 3.90 (m, 2H), 3.79 (m, 2H), 3.60 (t, J=5 Hz, 2H), 2.01 (m, 1H), 0.99 (m, 2H), 0.81 (m, 2H); MS [M−H]⁺=400.31.

### Compound 344

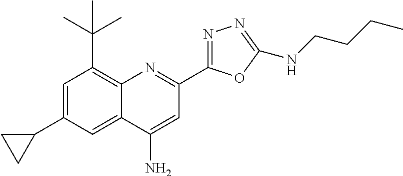

¹H-NMR (400 MHz, DMSO) δ 8.02 (s, 1H), 7.60 (s, 1H), 7.36 (s, 1H), 7.19 (s, 1H), 4.30 (br s, 2H), 3.34 (q, J=6 Hz, 2H), 2.03 (m, 1H), 1.57 (s, 9H), 1.50 (t, J=6 Hz, 2H), 0.997 (m, 2H), 0.88 (t, J=8 Hz, 3H), 0.82 (m, 2H); ¹⁹F NMR (376.1 MHz) δ −75.03 (s); MS [M−H]⁺=380.24.

### Compound 345

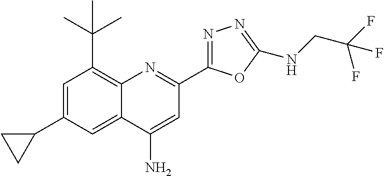

¹H-NMR (400 MHz, DMSO) δ 8.36 (m, 1H), 7.61 (s, 1H), 7.34 (s, 1H), 7.21 (s, 1H), 4.25 (app. quin, J=9 Hz, 2H), 2.02 (m, 1H), 0.98 (m, 2H), 0.81 (m, 2H); ¹⁹F NMR (376.1 MHz) δ −75.16 (s), −71.75 (t, J=9 Hz); MS [M−H]⁺=406.17.

### Compound 346

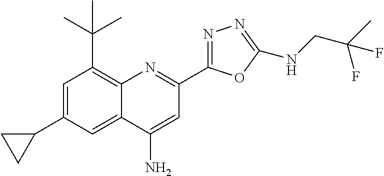

¹H-NMR (400 MHz, DMSO) δ 8.32 (m, 1H), 7.61 (s, 1H), 7.37 (s, 1H), 7.21 (s, 1H), 3.85 (td, J=15, 7 Hz), 2.02 (m, 1H), 1.57 (s, 9H), 1.22 (t, J=6 Hz, 3H), 0.98 (m, 2H), 0.80 (m, 2H); ¹⁹F NMR (376.1 MHz) δ −75.04 (s); MS [M−H]⁺=402.23.

### Compound 347

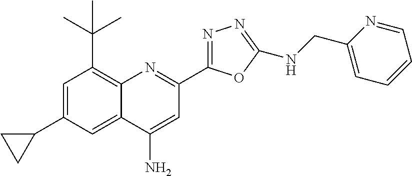

¹H-NMR (400 MHz, DMSO) δ 8.47 (m, 2H), 7.39 (t, J=6 Hz, 1H), 7.57 (s, 1H), 7.39 (d, J=7 Hz, 1H), 7.32 (s, 1H), 7.25 (m, 1H), 7.12 (s, 1H), 6.77 (s, 2H), 4.52 (d, J=6 Hz, 2H), 2.00 (m, 1H), 1.57 (s, 9H), 0.96 (m, 2H), 0.79 (m, 2H); ¹⁹F NMR (376.1 MHz) δ −73.95 (s); MS [M−H]⁺=415.31.

### Compound 348

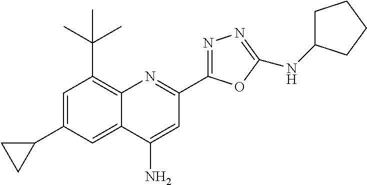

¹H-NMR (400 MHz, DMSO) δ 7.80 (d, J=6 Hz, 2H), 7.57 (s, 1H), 7.33 (s, 1H), 7.12 (s, 1H), 6.76 (s, 2H), 3.89 (m, 1H), 2.03 to 1.91 (m, 3H), 1.78 to 1.45 (m, 6 H) 1.59 (s, 9H), 0.97 (m, 2H), 0.79 (m, 2H); MS [M−H]⁺=392.28.

### Compound 349

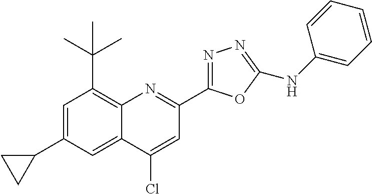

¹H-NMR (400 MHz, DMSO) δ 8.93 (t, 1H), 8.18 (s, 1H), 7.89 (s, 1H), 7.74 (m, 2H), 7.23 (d, 1H), 6.83 (d, 1H), 4.79 (d, 2H), 2.77 (s, 3H), 2.16 (m, 1H), 1.15 (m, 2H), 0.88 (m, 2H); MS [M−H]⁺=419.3.

### Compound 350

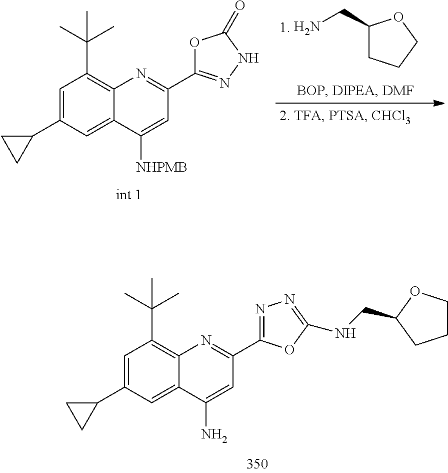

### Step 1:

Int 1 (150 mg, 0.338 mmol), dissolved in DMF (3 mL), was treated with diisopropyl ethylamine (120 μL, 0.676 mmol), (S)-(tetrahydrofuran-2-yl)methanamine (70 μL, 0676 mmol), and BOP reagent (164 mg, 0.372 mmol). The reaction was stirred at rt. After 2 h, the reaction mixture was diluted with water and extracted with EtOAc. The organic layer was concentrated and purified by flash chromatography.

### Step 2:

The crude product from the previous step was dissolved in chloroform (3 mL) and treated with 2 mL of TFA and 450 mg of PTSA. The reaction was stirred at rt overnight. The reaction mixture was concentrated, and the residue was dissolved in EtOAc and washed with sat. NaHCO₃ soln. The organic layer was dried over Na2SO4 and concentrated before purification by prep HPLC to give compound 350 as a white solid (7 mg, 5%).

¹H NMR (400 MHz, DMSO-d6) δ 8.03-7.94 (m, 1H), 7.57 (s, 1H), 7.33 (s, 1H), 7.11 (s, 1H), 4.01 (s, 1H), 3.74 (s, 1H), 3.62 (s, 1H), 3.53 (s, 3H), 3.25 (s, 2H), 2.05-1.95 (m, 1H), 1.95-1.86 (m, 1H), 1.81 (s, 2H), 1.59 (s, 10H), 0.98 (s, 2H), 0.81 (s, 2H); ¹⁹F NMR (376.1 MHz) δ −75.03 (TFA salt); MS [M+1-1]⁺=408.3; LC/MS RT=2.17 min.

### Compound 351-357

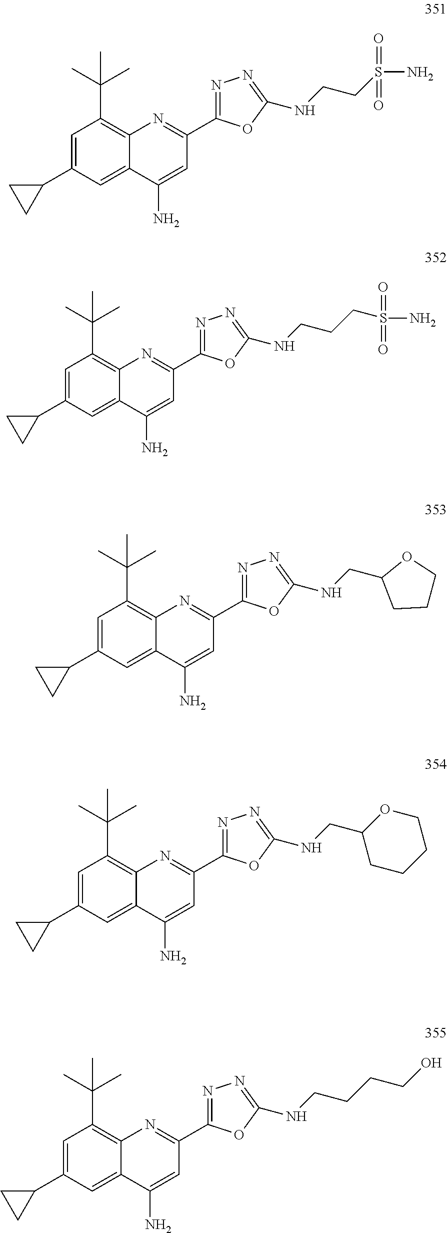

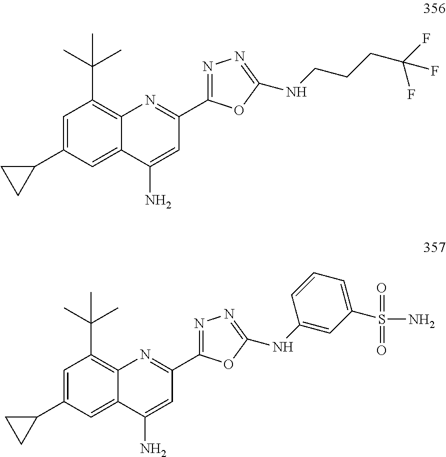

The compounds in the example were made according to procedures described previously.

351: ¹H NMR (400 MHz, DMSO-d6) δ 7.92 (s, 1H), 7.59 (s, 1H), 7.35 (s, 1H), 7.14 (s, 1H), 6.94 (s, 2H), 3.62 (m, 2H), 3.32 (m, 2H), 2.01 (m, 1H), 1.58 (s, 9H), 0.99 (m, 2H), 0.81 (m, 2H); ¹⁹F NMR (376.1 MHz) δ −75.43 (TFA salt); MS [M+H]⁺=431.3; LC/MS RT=1.99 min.

352: ¹H NMR (400 MHz, DMSO-d6) δ 7.58 (s, 2H), 7.44 (d, J=8.3 Hz, 1H), 7.35 (s, 2H), 7.13 (s, 2H), 7.07 (d, J=8.3 Hz, 1H), 6.78 (s, 4H), 3.35 (d, J=5.9 Hz, 4H), 3.09-2.99 (m, 4H), 2.00 (s, 6H), 1.59 (s, 17H), 0.98 (d, J=8.2 Hz, 4H), 0.80 (d, J=4.3 Hz, 4H); ¹⁹F NMR (376.1 MHz) δ −75.34 (TFA salt); MS [M+H]⁺=445.3; LC/MS RT=1.99 min.

353: ¹H NMR (400 MHz, DMSO-d6) δ 8.02 (s, 1H), 7.58 (s, 1H), 7.35 (s, 1H), 7.12 (s, 1H), 4.06-3.97 (m, 1H), 3.75 (dd, J=14.4, 6.9 Hz, 1H), 3.61 (dd, J=14.2, 7.7 Hz, 1H), 3.26 (t, J=5.9 Hz, 2H), 2.01 (s, 1H), 1.90 (d, J=11.3 Hz, 1H), 1.81 (d, J=6.5 Hz, 2H), 1.59 (s, 10H), 0.98 (d, J=8.3 Hz, 2H), 0.80 (d, J=5.0 Hz, 2H); ¹⁹F NMR (376.1 MHz) δ −75.31 (TFA salt); MS [M+H]⁺=408.3; LC/MS RT=2.25 min.

354: ¹H NMR (400 MHz, DMSO-d6) δ 8.08-7.95 (m, 1H), 7.59 (s, 1H), 7.35 (s, 1H), 7.11 (s, 1H), 3.85 (d, J=12.9 Hz, 1H), 3.46 (s, 1H), 3.31 (s, 1H), 3.22 (d, J=5.8 Hz, 2H), 2.01 (s, 1H), 1.76 (s, 1H), 1.59 (s, 10H), 1.43 (s, 3H), 1.18 (s, 1H), 0.98 (d, J=8.3 Hz, 2H), 0.80 (d, J=5.1 Hz, 2H); ¹⁹F NMR (376.1 MHz) δ −75.27 (TFA salt); MS [M+H]⁺=422.4; LC/MS RT=2.32 min.

355: ¹H NMR (400 MHz, DMSO-d6) δ 7.92-7.80 (m, 1H), 7.58 (s, 1H), 7.34 (s, 1H), 7.12 (s, 1H), 3.39 (t, J=6.4 Hz, 2H), 3.22 (d, J=6.3 Hz, 2H), 2.01 (s, 1H), 1.59 (s, 12H), 1.46 (s, 2H), 0.97 (d, J=7.7 Hz, 2H), 0.80 (s, 2H); ¹⁹F NMR (376.1 MHz) δ −74.83 (TFA salt); MS [M+H]⁺=396.3; LC/MS RT=2.46 min.

356: ¹H NMR (400 MHz, DMSO-d6) δ 7.95 (s, 1H), 7.58 (s, 1H), 7.34 (s, 1H), 7.13 (s, 1H), 3.31 (d, J=5.9 Hz, 2H), 2.33 (dd, J=16.3, 11.6 Hz, 3H), 2.01 (s, 1H), 1.85-1.75 (m, 2H), 1.59 (s, 10H), 0.98 (d, J=6.3 Hz, 2H), 0.80 (d, J=3.3 Hz, 2H); ¹⁹F NMR (376.1 MHz) δ −65.22, −75.30 (TFA salt); MS [M+H]⁺=434.3; LC/MS RT=2.37 min.

357: ¹H NMR (400 MHz, DMSO-d6) δ 11.01 (s, 1H), 8.17 (s, 1H), 7.84 (s, 1H), 7.60 (s, 1H), 7.53 (d, J=7.7 Hz, 1H), 7.45 (d, J=7.8 Hz, 1H), 7.38 (s, 3H), 7.23 (s, 1H), 2.02 (s, 1H), 1.63 (s, 9H), 1.00 (s, 2H), 0.82 (s, 2H); ¹⁹F NMR (376.1 MHz) δ −65.22, −75.09 (TFA salt); MS [M+H]⁺=479.3; LC/MS RT=2.31 min.

### Compound 358

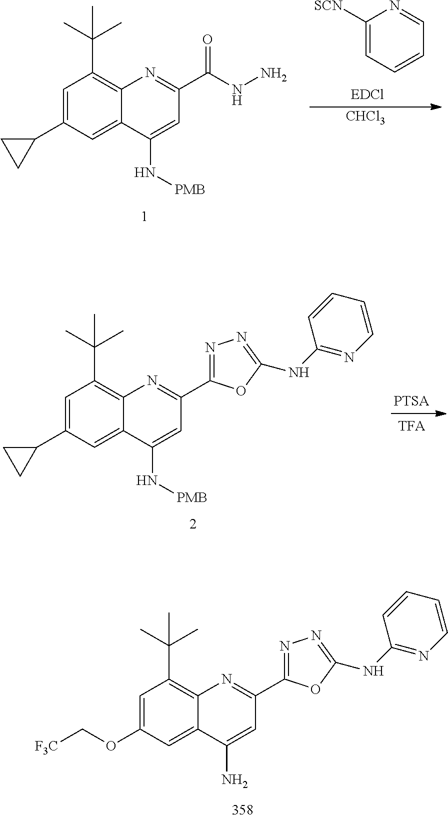

### Step 1:

The procedures described previously were followed to give 2 as a yellow solid.

### Step 2:

The procedures described previously were followed to give Compound 358 as a yellow solid (7 mg, 61%).

¹H NMR (400 MHz, DMSO-d6) δ 8.27 (d, J=5.1 Hz, 1H), 7.85 (s, 2H), 7.61 (s, 1H), 7.37 (s, 1H), 7.20 (s, 1H), 7.03 (s, 1H), 2.85 (s, 1H), 2.69 (s, 1H), 1.62 (s, 9H); ¹⁹F NMR (376.1 MHz) δ −75.36 (TFA salt); MS [M+H]⁺=401.2; LC/MS RT=2.38 min.

### Compound 359

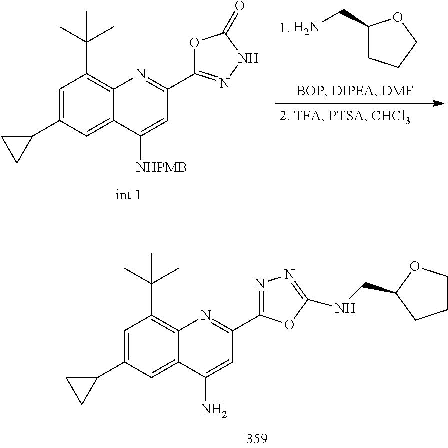

### Step 1:

Int 1 (150 mg, 0.338 mmol), dissolved in DMF (3 mL), was treated with diisopropyl ethylamine (120 μL, 0.676 mmol), (S)-(tetrahydrofuran-2-yl)methanamine (70 μL, 0676 mmol), and BOP reagent (164 mg, 0.372 mmol). The reaction was stirred at rt. After 2 h, the reaction mixture was diluted with water and extracted with EtOAc. The organic layer was concentrated and purified by flash chromatography.

### Step 2:

The crude product from the previous step was dissolved in chloroform (3 mL) and treated with 2 mL of TFA and 450 mg of PTSA. The reaction was stirred at it overnight. The reaction mixture was concentrated, and the residue was dissolved in EtOAc and washed with sat. NaHCO₃ soln. The organic layer was dried over Na2SO4 and concentrated before purification by prep HPLC to give compound 359 as a white solid (7 mg, 5%).

¹H NMR (400 MHz, DMSO-d6) δ 8.03-7.94 (m, 1H), 7.57 (s, 1H), 7.33 (s, 1H), 7.11 (s, 1H), 4.01 (s, 1H), 3.74 (s, 1H), 3.62 (s, 1H), 3.53 (s, 3H), 3.25 (s, 2H), 2.05-1.95 (m, 1H), 1.95-1.86 (m, 1H), 1.81 (s, 2H), 1.59 (s, 10H), 0.98 (s, 2H), 0.81 (s, 2H); ¹⁹F NMR (376.1 MHz) δ −75.03 (TFA salt); MS [M+H]⁺=408.3; LC/MS RT=2.17 min.

### Compound 360

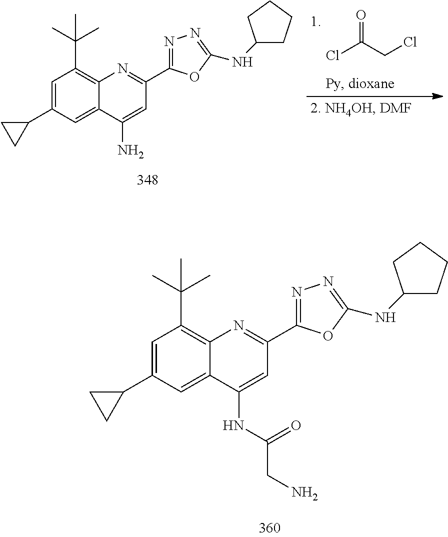

### Step 1:

Compound 348 (99 mg, 0.253 mmol), suspended in dioxane (5 mL) and cooled to 0° C., was treated with pyridine (61 μL, 0.760 mmol) followed by chloroacetyl chloride (60 μL, 0.760 mmol). The reaction mixture was warmed to rt, stirred for 3 h, and concentrated.

### Step 2:

The residue from the previous step (30 mg, 0.064 mmol) was dissolved in DMF (5 mL) and treated with ammonium hydroxide (2 mL). The reaction mixture was stirred at rt overnight. It was then filtered through a syringe filter before purification by prep HPLC to give compound 360 as a white solid (15 mg, 50%).

¹H NMR (400 MHz, DMSO-d6) δ 10.55 (s, 1H), 8.60 (s, 1H), 8.04 (s, 1H), 7.76 (s, 1H), 7.46 (s, 1H), 4.03 (s, 3H), 3.97-3.87 (m, 2H), 2.17-2.05 (m, 1H), 1.98-1.87 (m, 2H), 1.72-1.65 (m, 2H), 1.57-1.48 (m, 2H), 1.08 (s, 2H), 0.89 (s, 2H);

¹⁹F NMR (376.1 MHz) δ −74.51 (TFA salt); MS [M+H]⁺=449.3; LC/MS RT=2.21 min.

### Compound 361

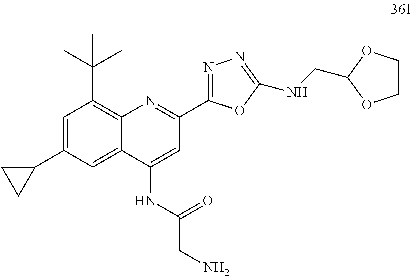

The compound in the example was made according to procedures described previously from compound 342.

¹H NMR (400 MHz, DMSO-d6) δ 10.56 (s, 1H), 8.59 (s, 1H), 8.26 (s, 1H), 8.14 (s, 2H), 7.77 (s, 1H), 7.46 (s, 1H), 5.03 (s, 1H), 4.03 (s, 2H), 3.91 (s, 2H), 3.80 (s, 3H), 3.38 (s, 2H), 2.18-2.05 (m, 1H), 1.62 (s, 9H), 1.08 (s, 2H), 0.89 (s, 2H); ¹⁹F NMR (376.1 MHz) δ −74.43 (TFA salt); MS [M+H]⁺=467.4; LC/MS RT=1.93 min.

### Compound 362

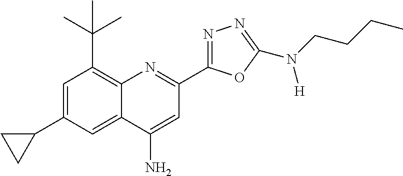

¹H NMR (400 MHz, dmso) δ 7.88 (s, 1H), 7.58 (s, 1H), 7.34 (s, 1H), 7.12 (s, 1H), 3.22 (dd, J=12.9, 6.8 Hz, 2H), 2.07-1.91 (m, 1H), 1.68-1.43 (m, 10H), 1.39-1.25 (m, 2H), 1.01-0.91 (m, 2H), 0.88 (t, J=7.4 Hz, 3H), 0.79 (dd, J=5.7, 3.9 Hz, 2H); ¹⁹F NMR (376 MHz, dmso) δ −75.31; MS [M+H]⁺=437.16.

### Compound 363

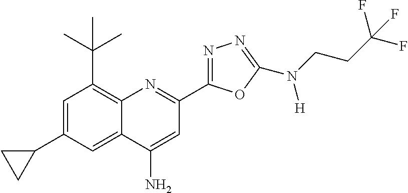

¹H NMR (400 MHz, dmso) δ 8.09 (s, 1H), 7.58 (s, 1H), 7.34 (s, 1H), 7.14 (s, 1H), 3.48 (dd, J=12.9, 6.7 Hz, 2H), 2.10-1.89 (m, 1H), 1.58 (s, 8H), 1.06-0.85 (m, 2H), 0.87-0.68 (m, 2H); ¹⁹F NMR (376 MHz, dmso) δ −64.29, −64.32, −64.35, −75.37; MS [M+H]⁺=437.16.

### Compound 364

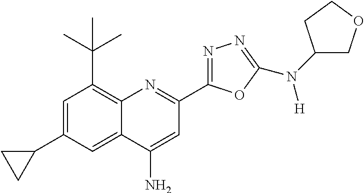

¹H NMR (400 MHz, dmso) δ 8.13 (s, 1H), 7.59 (s, 1H), 7.35 (s, 1H), 7.13 (s, 1H), 4.17 (s, 1H), 3.92-3.73 (m, 2H), 3.76 3.60 (m, 2H), 2.17 (td, J=15.3, 7.7 Hz, 1H), 1.99 (ddd, J=21.1, 12.3, 3.6 Hz, 2H), 1.59 (s, 8H), 1.09-0.88 (m, 2H), 0.87-0.73 (m, 2H); ¹⁹F NMR (376 MHz, dmso) δ −75.38; MS [M+H]⁺=437.16.

### Compound 365

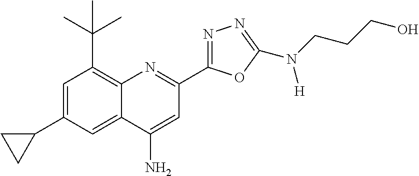

¹H NMR (400 MHz, dmso) δ 7.90 (s, 1H), 7.59 (s, 1H), 7.35 (s, 1H), 7.12 (s, 1H), 3.47 (t, J=6.2 Hz, 2H), 3.29 (dd, J=12.6, 6.6 Hz, 2H), 2.01 (ddd, J=13.4, 8.5, 5.2 Hz, 1H), 1.79-1.64 (m, 2H), 1.58 (s, 8H), 1.05-0.86 (m, 2H), 0.85-0.70 (m, 2H; ¹⁹F NMR (376 MHz, dmso) δ −75.41.

(376 MHz, dmso) δ −75.41; MS [M+H]⁺=437.16.

### Compound 366

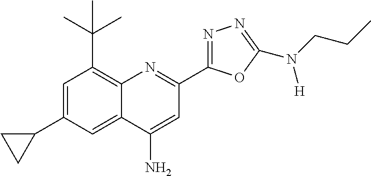

¹H NMR (400 MHz, dmso) δ 7.91 (s, 1H), 7.58 (s, 1H), 7.34 (s, 1H), 7.12 (s, 1H), 3.18 (dd, J=13.1, 6.6 Hz, 2H), 2.00 (td, J=8.4, 4.4 Hz, 1H), 1.66-1.49 (m, 11H), 1.01-0.92 (m, 2H), 0.89 (t, J=7.4 Hz, 3H), 0.84-0.73 (m, 2H); ¹⁹F NMR (376 MHz, dmso) δ −75.32

### Compound 367

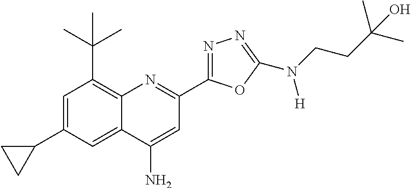

¹H NMR (400 MHz, dmso) δ 7.77 (s, 1H), 7.58 (s, 1H), 7.34 (s, 1H), 7.12 (s, 1H), 3.31 (dt, J=10.6, 5.5 Hz, 2H), 2.01 (t, J=5.0 Hz, 1H), 1.75-1.62 (m, 2H), 1.58 (s, 9H), 1.11 (s, 6H), 1.04-0.86 (m, 2H), 0.79 (dd, J=5.8, 4.0 Hz, 2H); ¹⁹F NMR (376 MHz, dmso) δ −75.27; MS [M+H]⁺=437.16.

### Compound 368

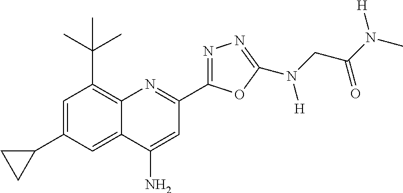

¹H NMR (400 MHz, dmso) δ 8.13 (s, 1H), 7.95 (d, J=4.6 Hz, 1H), 7.58 (s, 1H), 7.34 (s, 1H), 7.13 (s, 1H), 3.79 (d, J=5.9 Hz, 2H), 2.58 (d, J=4.6 Hz, 3H), 2.01 (t, J=4.8 Hz, 1H), 1.59 (s, 9H), 0.97 (dd, J=7.1, 5.1 Hz, 2H), 0.83-0.74 (m, 2H); ¹⁹F NMR (376 MHz, dmso) δ −75.29; MS [M+H]⁺=437.16.

### Compound 369

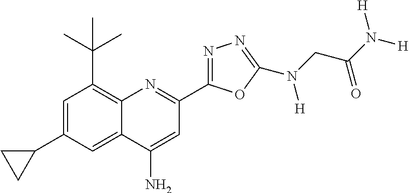

¹H NMR (400 MHz, dmso) δ 8.07 (s, 1H), 7.59 (s, 1H), 7.47 (s, 1H), 7.35 (s, 1H), 7.10 (d, J=20.1 Hz, 2H), 3.78 (d, J=5.6 Hz, 2H), 2.01 (td, J=8.4, 4.2 Hz, 1H), 1.59 (s, 8H), 1.05-0.86 (m, 2H), 0.91-0.72 (m, 2H); ¹⁹F NMR (376 MHz, dmso) δ −75.39; MS [M+H]⁺=437.16.

### Compound 370

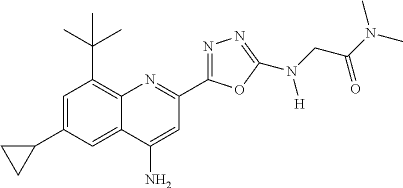

¹H NMR (400 MHz, dmso) δ 7.99 (s, 3H), 7.58 (s, 3H), 7.35 (s, 3H), 7.13 (s, 3H), 4.08 (d, J=4.8 Hz, 5H), 2.98 (s, 7H), 2.82 (s, 7H), 2.07-1.91 (m, 3H), 1.59 (s, 21H), 1.05-0.87 (m, 5H), 0.88-0.69 (m, 5H); ¹⁹F NMR (376 MHz, dmso) δ −75.39; MS [M+H]⁺=437.16.

### Compound 371

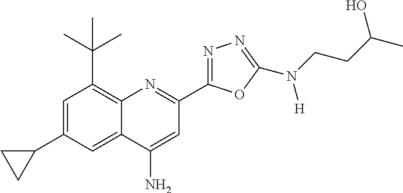

¹H NMR (400 MHz, dmso) δ 7.78 (s, 1H), 7.57 (s, 1H), 7.33 (s, 1H), 7.12 (s, 1H), 3.68 (d, J=6.1 Hz, 1H), 3.27 (dd, J=12.9, 7.0 Hz, 2H), 2.00 (s, 2H), 1.73-1.42 (m, 12H), 1.06 (d, J=6.2 Hz, 3H), 1.00-0.89 (m, 2H), 0.80 (t, J=5.5 Hz, 2H); ¹⁹F NMR (376 MHz, dmso) δ −74.78; MS [M+H]⁺=437.16.

### Compound 372

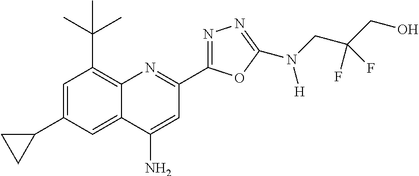

Prepared analogously to compound 33, TFA/pTosH removed both the PMB and OTBDPS protecting groups simultaneously.

¹H NMR (400 MHz, dmso) δ 8.31 (s, 1H), 7.57 (s, 1H), 7.33 (s, 1H), 7.12 (s, 1H), 5.57 (t, J=6.3 Hz, 1H), 3.68 (ddd, J=22.0, 19.8, 10.1 Hz, 4H), 2.08-1.91 (m, 1H), 1.59 (s, 8H), 1.10-0.91 (m, 2H), 0.84-0.70 (m, 2H); ¹⁹F NMR (376 MHz, dmso) δ −112.70, −112.74, −112.78, −112.82, −112.85; MS [M+H]⁺=418.28.

### Example 39

### Compound 373

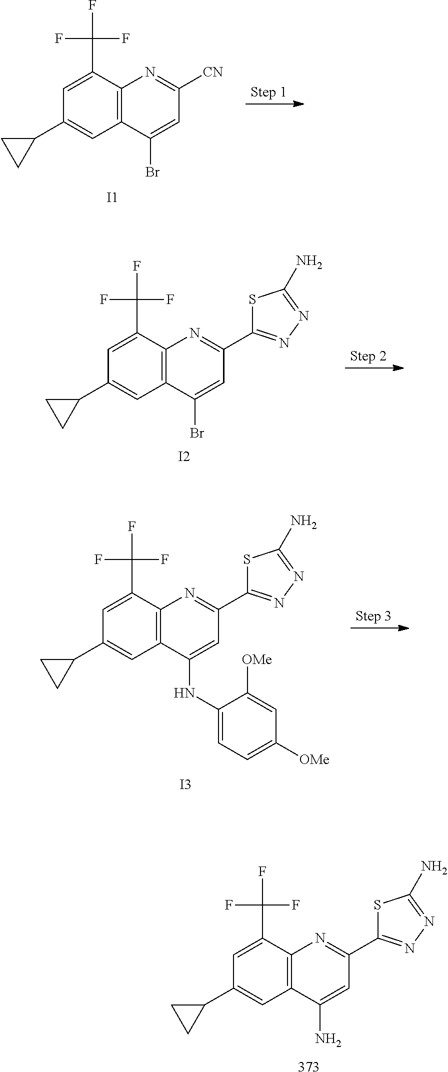

### Step 1

To 4-bromo-6-cyclopropyl-4-methyl-8-trifluoromethyl-quinoline-2-carbonitrile (11) (100 mg, 0.28 mmol) dissolved in 1 mL TFA was added 25 mg (1.1 equiv) thiosemicarbazide. The reaction was heated at 65° C. for 1 h, at which time the solvent was removed by co-evaporation with toluene and the residue purified by flash chromatography to give 63 mg of final product (12). MS [M+H]⁺=415.

### Step 2

Compound I2 (104 mg, 0.25 mmol) was dissolved in 2 mL of 2,4-dimethoxybenzylamine. The mixture was stirred at 80° C. for 5 h. The reaction mixture was cooled to room temperature and diluted in ethyl acetate. The solution was washed with a concentrated ammonium chloride solution, followed by a water wash and a brine wash. The solution was dried over Na₂SO₄ and concentrated under vacuum. The resulting crude was purified by flash chromatography to give 71 mg of final product (13). MS [M+H]⁺=488.

### Step 3

Compound I3 (60 mg, 0.12 mmol) was dissolved in 2 ml of DCE. 1 mL of TFA was added and the reaction was stirred at room temperature for 1 h. The mixture was concentrated under vacuum and the crude was purified by HPLC to give 21 mg of compound 373. ¹H-NMR (400 MHz, DMSO) δ δ 8.01 (s, 1H), 7.78 (s, 1H), 7.51 (s, 2H), 7.27 (s, 1H), 2.12 (s, 1H), 1.08-0.94 (m, 2H), 0.86 (dd, J=8.0, 3.1 Hz, 2H). MS [M+H]⁺=352.

### Compound 374

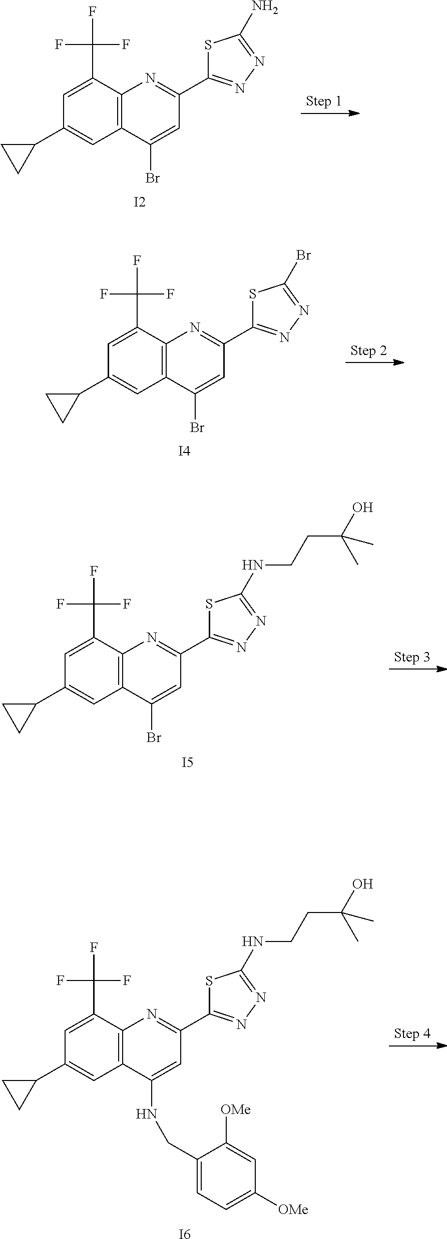

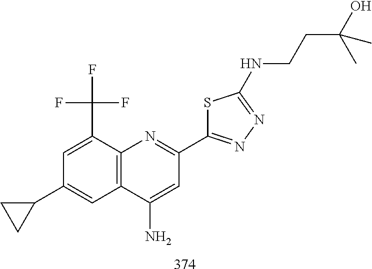

### Step 1

A similar procedure was used as in step 5 of the synthesis for compound 286 to give compound I4. MS [M+H]⁺=479.

### Step 2

A similar procedure was used as in step 6 of the synthesis for compound 286 to give compound I5. MS [M+H]⁺=502.

### Step 3

A similar procedure was used as in step 2 of the synthesis for compound S5 to give compound I6. MS [M+H]⁺=588.

### Step 4

A similar procedure was used as in step 3 of the synthesis for compound 286 to give compound 374. ¹H-NMR (400 MHz, DMSO) δ 8.01 (s, 1H), 7.95 (s, 1H), 7.78 (s, 1H), 7.28 (s, 1H), 7.14 (s, 2H), 3.36 (s, 2H), 2.21-2.00 (m, 1H), 1.76-1.58 (m, 2H), 1.11 (s, 6H), 1.08-0.96 (m, 2H), 0.86 (q, J=4.4 Hz, 2H). MS [M+H]⁺=438.

### Compound 375

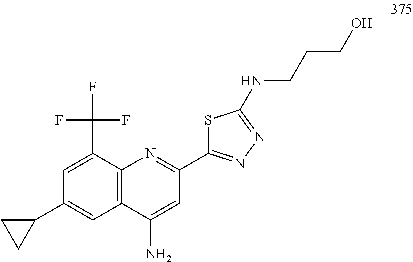

Compound 375 was prepared in a similar manner as compound 374. ¹H-NMR (400 MHz, DMSO) δ 8.13 (s, 1H), 8.01 (s, 1H), 7.79 (s, 1H), 7.28 (s, 1H), 3.47 (t, J=6.2 Hz, 2H), 3.36 (d, J=4.4 Hz, 2H), 2.21-2.06 (m, 1H), 1.72 (p, J=6.6 Hz, 2H), 1.03 (dt, J=6.2, 4.3 Hz, 2H), 0.93-0.83 (m, 2H). MS [M+H]⁺=410.

### Compound 376

Compounds 377 and 378 are obtained in the reaction between various C2 carboxylates and n-butyl thisosemicarbazide, following a method described elsewhere in this patent. Compound 376 results from the treatment of 1012 with DMB-amine at elevated temperature followed by reaction with TFA.

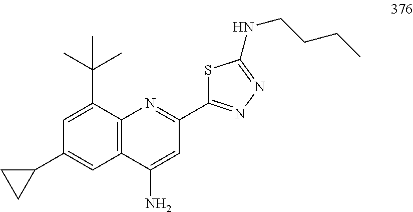

Compound 376: Obtained in 30% yield after HPLC purification. ¹H-NMR (400 MHz, CDCl₃) δ 8.02 (s, 1H), 7.62 (s, 1H), 7.40 (s, 1H), 3.53 (m, 2H), 1.72 (m, 2H), 1.62 (s, 9H), 1.42 (m, 2H), 1.12-1.00 (m, 2H), 0.95 (m, 3H), 0.94-0.84 (m, 2H). MS [M+H]⁺=396.

### Compound 377-378

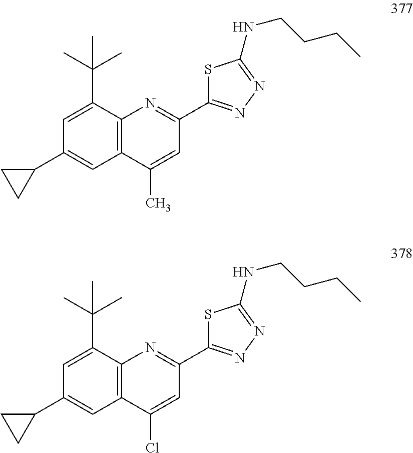

Compound 377: Obtained in 15% yield after HPLC purification. ¹H-NMR (400 MHz, CDCl₃) δ 8.00 (s, 1H), 7.63 (s, 1H), 7.45 (s, 1H), 3.49 (m, 2H), 2.74 (s, 3H), 1.71 (m, 2H), 1.66 (s, 9H), 1.46 (m, 2H), 1.13-1.00 (m, 2H), 0.96 (m, 3H), 0.94-0.84 (m, 2H). MS [M+H]⁺=395.

Compound 378: Obtained in 20% yield after HPLC purification. ¹H-NMR (400 MHz, CDCl₃) δ 8.01 (s, 1H), 7.64 (s, 1H), 7.42 (s, 1H), 3.51 (m, 2H), 1.70 (m, 2H), 1.64 (s, 9H), 1.44 (m, 2H), 1.12-1.00 (m, 2H), 0.95 (m, 3H), 0.94-0.84 (m, 2H). MS [M+H]⁺=415.

### Compound 379

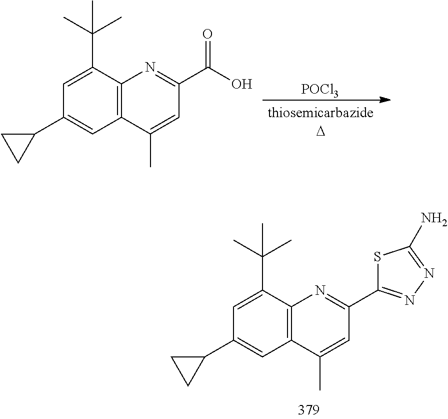

Compound 379 resulted from the treatment of the corresponding carboxylate with POCl₃ and thiosemicarbazide at elevated temperature.

¹H-NMR (400 MHz, CD₃CN) δ 8.04 (s, 1H), 7.62 (s, 1H), 7.44 (s, 1H), 2.71 (s, 3H), 1.61 (s, 9H), 1.07-1.00 (m, 2H), 0.88-0.84 (m, 2H). MS [M+H]⁺=339.

### Compounds 380-382

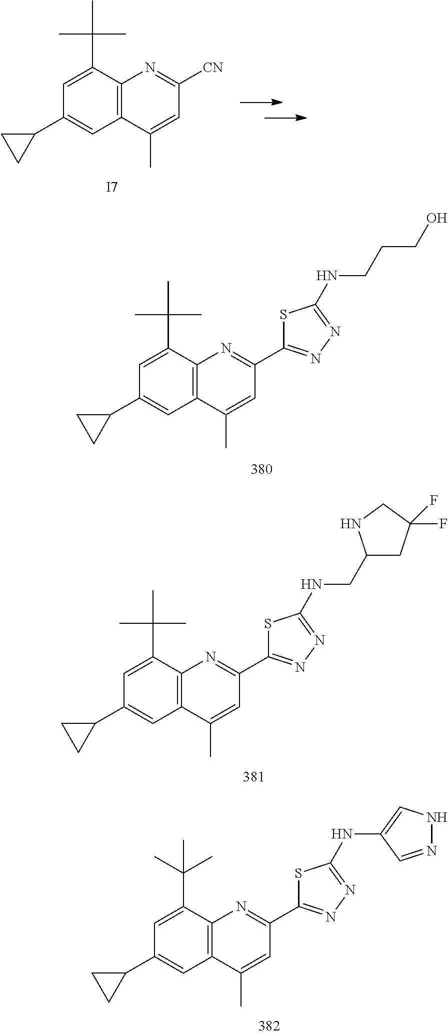

Compounds 380, 381 and 382 were prepared from intermediate 17 in a similar manner as the preparation of Compound 286 from intermediate E.

Compound 380: ¹H-NMR (400 MHz, CDCl₃) δ 7.88 (d, J=6.2 Hz, 1H), 7.52 (t, J=5.9 Hz, 1H), 7.44 (d, J=1.8 Hz, 1H), 3.87 (dd, J=22.2, 16.7 Hz, 2H), 3.77-3.58 (m, 2H), 2.88-2.58 (m, 2H), 2.26-1.89 (m, 4H), 1.76-1.49 (m, 9H), 1.18-1.01 (m, 2H), 0.96-0.73 (m, 2H). MS [M+H]⁺=397.

Compound 381: ¹H-NMR (400 MHz, DMSO) δ 8.55-8.23 (m, 1H), 7.92 (d, J=7.2 Hz, 1H), 7.58 (t, J=10.3 Hz, 1H), 7.39 (t, J=10.1 Hz, 1H), 4.28-3.97 (m, 1H), 3.93-3.49 (m, 3H), 2.82 (d, J=23.8 Hz, 1H), 2.79-2.63 (m, 3H), 2.51-2.41 (m, 3H), 2.13 (ddd, J=13.4, 8.3, 5.1 Hz, 1H), 1.74-1.44 (m, 9H), 1.10-0.93 (m, 2H), 0.91-0.74 (m, 2H). MS [M+H]⁺=458.

Compound 382: ¹H-NMR (400 MHz, DMSO) δ 7.99 (s, 1H), 7.77 (s, 2H), 7.58 (s, 1H), 7.38 (s, 1H), 2.70 (d, J=6.3 Hz, 3H), 2.15 (s, 1H), 1.61 (d, J=6.2 Hz, 9H), 1.04 (s, 2H), 0.85 (s, 2H). MS [M+H]⁺=405.

### Compounds 383-384

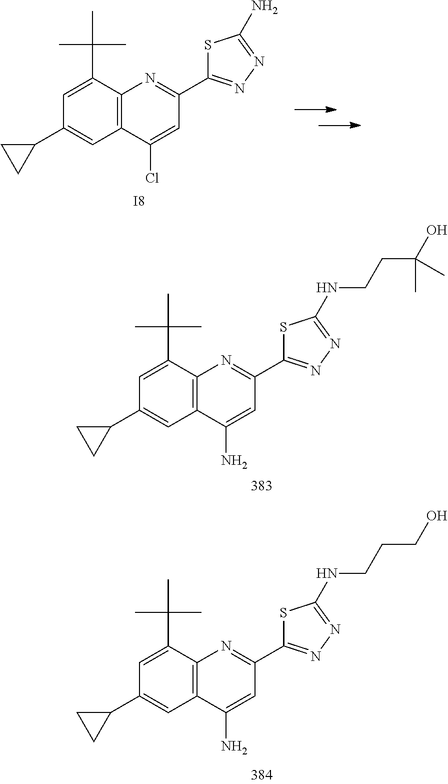

Compounds 383 and 384 were prepared from intermediate 18 in a similar manner described previously.

Compound 383: ¹H-NMR (400 MHz, CDCl₃) δ 7.78 (s, 1H), 7.61 (s, 1H), 7.47 (d, J=28.2 Hz, 1H), 3.65 (dd, J=14.2, 7.5 Hz, 2H), 3.04 (s, 1H), 2.90 (d, J=32.5 Hz, 1H), 2.21-1.99 (m, 1H), 1.59 (s, 9H), 1.33 (d, J=5.1 Hz, 6H), 1.17-1.03 (m, 2H), 0.91 (t, J=15.1 Hz, 2H). MS [M+H]⁺=426.

Compound 384: ¹H-NMR (400 MHz, DMSO) δ 7.97-7.76 (m, 1H), 7.65-7.42 (m, 1H), 7.34 (d, J=20.9 Hz, 1H), 6.77 (s, 2H), 3.46 (t, J=6.2 Hz, 2H), 3.41-3.17 (m, 2H), 2.08-1.85 (m, 1H), 1.80-1.64 (m, 2H), 1.56 (s, 9H), 1.00-0.87 (m, 2H), 0.84-0.60 (m, 2H). MS [M+H]⁺=398.

### Compounds 385-386

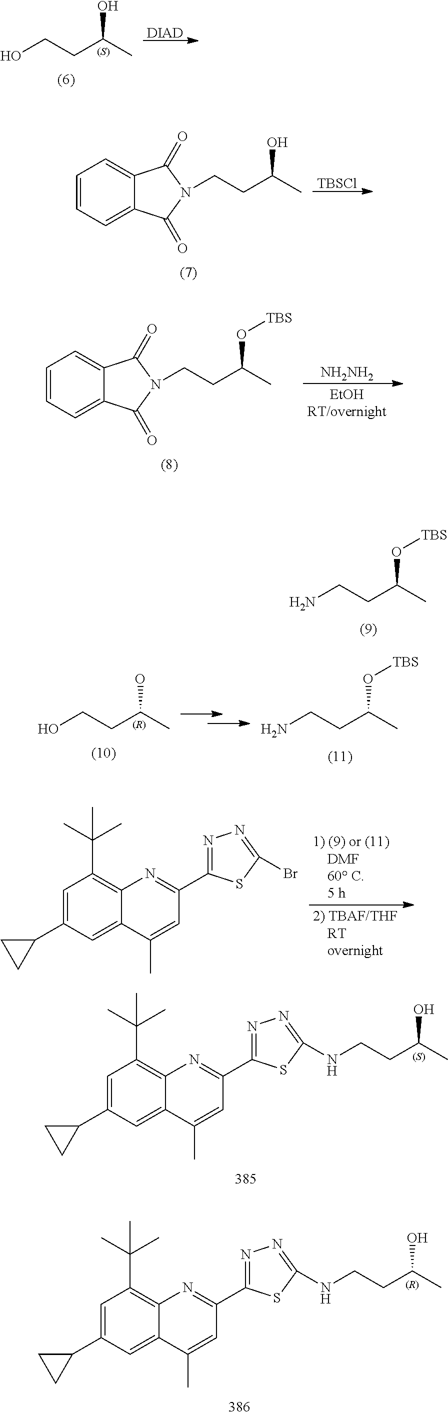

Diol (6) (1 g, 11.1 mmol)) in THF (60 mL) with PPh3 (3.2 g, 11.2 mmol) and phthalimide (1.63 g, 11.1 mmol) was cooled to 0° C. under N². DEAD (40% in toluene, 5.53 mL, 12.2 mmol) was added dropwise. The mixture was stirred form 0° C. to RT for 4 h. After completion of the reaction, it was concentrated. The crude product was purified by flash chromatography on silica gel with EtOAc/Hexane to give compound (7).

Compound (7) was dissolved in DCM (100 mL) with imidazole (1.52 g, 22.2 mmol). It was cooled to 0° C. under N₂. TBS-Cl (2.07 g, 13.3 mmol) was added dropwise. The mixture was stirred form 0° C. to RT for 18 h. After completion of the reaction, it was concentrated. The crude product was purified by flash chromatography on silica gel with EtOAc/Hexane to give compound (8), yield 3.0 g, 81% from (6).

Compound (8) (1.0 g, 3.0 mmol) was dissolved in EtOH (30 mL) with hydrazine hydrate (0.73 mL, 15 mmol). It was stirred at RT under N₂ overnight. The solid was filtered off. The filtrate was concentrated. The residue was dissolved in Ether, washed with water, brine and dried (Na₂SO₄). After concentration, it gave a colorless liquid as amine product (9).

Compound 385: was prepared from compound 8 and bromo-intermediate as described previously.

¹H-NMR (400 MHz, CDCl₃) δ 11.35 (m, 1H), 7.87 (s, 1H), 7.53 (s, 1H), 7.43 (s, 1H), 4.03 (m, 1H), 3.66-3.57 (m, 2H), 2.72 (s, 3H), 2.11 (m, 2H), 1.99 (m, 1H), 1.86 (m, 1H), 1.65 (s, 9H), 1.30 (d, 3H), 1.09 (m, 2H), 0.85 (m, 2H); ¹⁹F NMR (376.1 MHz) δ −76.5 (s); MS [M+H]⁺=411.3

Compound 386: was prepared in a manner similar to compound 385.

¹H-NMR (400 MHz, CDCl₃) δ 11.35 (m, 1H), 7.87 (s, 1H), 7.53 (s, 1H), 7.43 (s, 1H), 4.05 (m, 1H), 3.60 (m, 2H), 2.73 (s, 3H), 2.30-1.80 (m, 4H), 1.64 (s, 9H), 1.30 (d, 3H), 1.10 (m, 2H), 0.85 (m, 2H); ¹⁹F NMR (376.1 MHz) δ −76.5 (s); MS [M+H]⁺=411.3

### Example 40

### Compounds 387-390

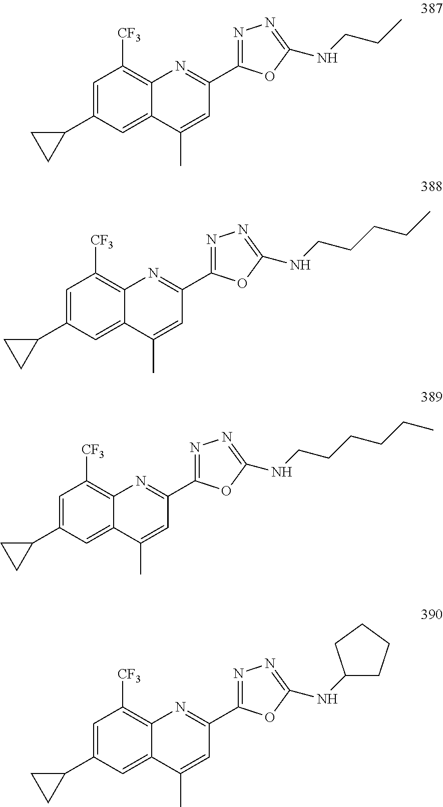

The compounds in the example were made according to procedures described in example 29.

387: ¹H NMR (400 MHz, DMSO-d6) δ 8.16 (t, J=5.5 Hz, 1H), 8.04 (d, J=15.3 Hz, 2H), 7.86 (s, 1H), 3.21 (dd, J=12.8, 6.5 Hz, 2H), 2.75 (s, 3H), 2.26 (d, J=5.1 Hz, 1H), 1.58 (dd, J=14.2, 7.2 Hz, 2H), 1.14-1.02 (m, 2H), 0.92 (dt, J=14.8, 5.9 Hz, 5H); ¹⁹F NMR (376.1 MHz) δ −58.82, −75.25 (TFA salt); MS [M+H]⁺=377.2; LC/MS RT=2.54 min.

388: ¹H NMR (400 MHz, DMSO-d6) δ 8.14 (d, J=5.4 Hz, 1H), 8.06 (s, 1H), 8.02 (s, 1H), 7.86 (s, 1H), 3.24 (dd, J=12.9, 6.9 Hz, 2H), 2.75 (s, 3H), 2.27 (s, 1H), 1.57 (s, 2H), 1.33-1.25 (m, 4H), 1.09 (d, J=8.3 Hz, 2H), 0.94 (d, J=6.5 Hz, 2H), 0.86 (d, J=6.9 Hz, 3H); ¹⁹F NMR (376.1 MHz) δ −58.82, −74.74 (TFA salt); MS [M+H]⁺=405.2; LC/MS RT=2.65 min.

389: ¹H NMR (400 MHz, DMSO-d6) δ 8.13 (s, 1H), 8.05 (s, 1H), 8.02 (s, 1H), 7.86 (s, 1H), 3.28 (s, 2H), 3.24 (dd, J=12.7, 6.7 Hz, 2H), 2.75 (s, 3H), 2.27 (s, 1H), 1.55 (d, J=7.4 Hz, 2H), 1.26 (d, J=6.7 Hz, 6H), 1.09 (d, J=6.4 Hz, 2H), 0.94 (d, J=6.8 Hz, 2H), 0.84 (t, J=6.7 Hz, 3H); ¹⁹F NMR (376.1 MHz) δ −58.82; MS [M+H]⁺=419.3; LC/MS RT=2.71 min.

390: ¹H NMR (400 MHz, DMSO-d6) δ 8.15 (d, J=6.6 Hz, 1H), 8.06 (s, 1H), 8.02 (s, 1H), 7.86 (s, 1H), 3.95 (d, J=6.7 Hz, 1H), 2.75 (s, 3H), 2.27 (s, 1H), 1.89 (s, 2H), 1.68 (s, 2H), 1.56 (d, J=12.5 Hz, 4H), 1.13-1.06 (m, 2H), 0.93 (d, J=6.8 Hz, 2H); ¹⁹F NMR (376.1 MHz) δ −58.80, −74.98 (TFA salt); MS [M+H]⁺=403.2; LC/MS RT=2.56 min.

### Example 41

### Compounds 391-392

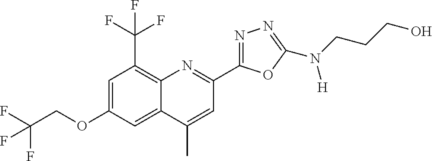

Prepared analogously to compound 280.

### Compound 391

¹H NMR (400 MHz, dmso) δ 8.09 (m, 2H), 7.93 (d, J=2.7 Hz, 1H), 7.86 (d, J=2.7 Hz, 1H), 5.08 (q, J=8.8 Hz, 2H), 3.47 (t, J=6.2 Hz, 2H), 3.31 (dd, J=12.7, 6.9 Hz, 2H), 2.76 (s, 3H), 1.72 (p, J=6.5 Hz, 2H); ¹⁹F NMR (376 MHz, dmso) 6-59.17, −72.85, −72.88, −72.90, −75.03); MS [M+H]⁺=437.16.

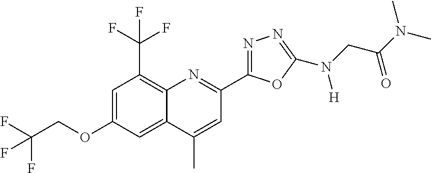

### Compound 392

¹H NMR (400 MHz, dmso) δ 8.25 (t, J=6.3 Hz, 1H), 8.10 (s, 1H), 7.94 (d, J=2.6 Hz, 1H), 7.86 (d, J=2.5 Hz, 1H), 5.08 (q, J=8.8 Hz, 2H), 4.12 (d, J=6.2 Hz, 2H), 2.98 (s, 3H), 2.83 (s, 3H), 2.76 (s, 3H); ¹⁹F NMR (376 MHz, dmso) δ −59.19, −72.85, −72.87, −72.90, −74.96; MS [M+H]⁺=437.16.

### Example 42

### Compounds 393-423

### Compound 393

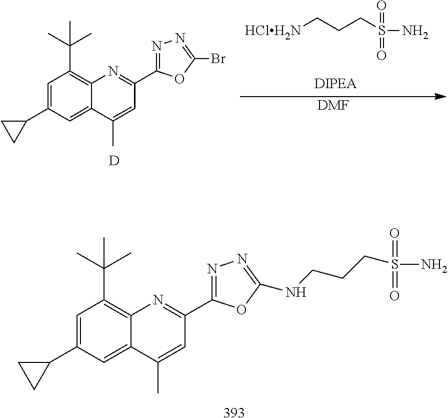

Compound D from Example 31 (150 mg, 0.389 mmol), dissolved in DMF (3 mL), was treated with 3-aminopropane-1-sulfonamide hydrochloride (102 mg, 0.583 mmol), followed by diisopropyl ethylamine (135 μL, 0.777 mmol). The reaction mixture was heated at 45° C. for 4 h. The reaction mixture was then concentrated and the residue was suspended in DMF and filtered through a syringe filter before purification by prep HPLC to give compound 393 as a white solid (4 mg, 2%).

¹H NMR (400 MHz, DMSO-d6) δ 8.09 (s, 1H), 7.87 (s, 1H), 7.56 (s, 1H), 7.38 (s, 1H), 6.78 (s, 2H), 3.38 (d, J=5.9 Hz, 2H), 3.10-3.00 (m, 2H), 2.67 (s, 3H), 2.13 (s, 1H), 2.01 (s, 2H), 1.62 (s, 9H), 1.03 (d, J=8.4 Hz, 2H), 0.84 (d, J=6.8 Hz, 2H); ¹⁹F NMR (376.1 MHz) δ −75.04 (TFA salt); MS [M+H]⁺=444.3; LC/MS RT=2.31 min.

### Compounds 394-395

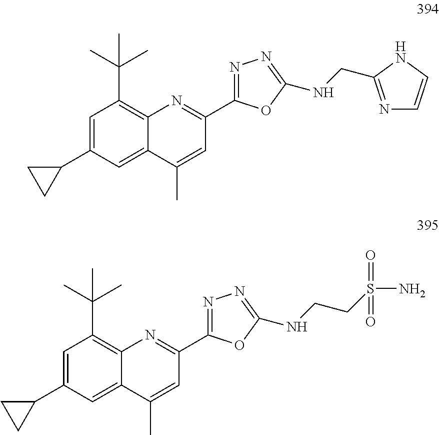

The compounds in the example were made according to procedures described previously.

394: ¹H NMR (400 MHz, DMSO-d6) δ 8.91-8.83 (m, 1H), 7.89 (s, 1H), 7.58 (s, 3H), 7.39 (s, 1H), 4.77 (s, 2H), 2.68 (s, 3H), 2.18-2.07 (m, 1H), 1.60 (s, 9H), 1.10-1.00 (m, 2H), 0.88-0.81 (m, 2H); ¹⁹F NMR (376.1 MHz) δ −74.03 (TFA salt); MS [M+H]⁺=403.2; LC/MS RT=2.13 min.

395: ¹H NMR (400 MHz, DMSO-d6) δ 8.14 (s, 1H), 7.89 (s, 1H), 7.57 (s, 1H), 7.38 (s, 1H), 6.96 (s, 2H), 3.64 (s, 2H), 3.33 (s, 3H), 2.68 (s, 3H), 2.17-2.08 (m, 1H), 1.61 (s, 9H), 1.05 (s, 2H), 0.85 (s, 2H); ¹⁹F NMR (376.1 MHz) δ −74.54 (TFA salt); MS [M+H]⁺=430.2; LC/MS RT=2.32 min.

### Compound 396

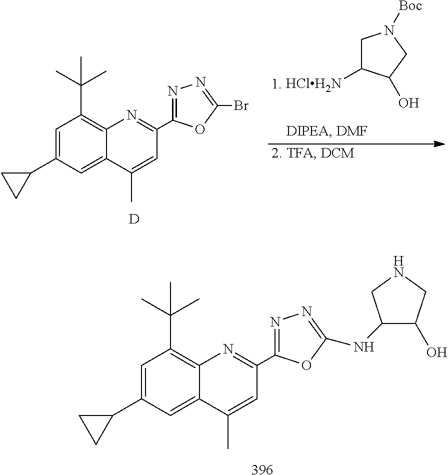

### Step 1:

Compound D from Example 31 (150 mg, 0.389 mmol), dissolved in DMF (4 mL) was treated with tert-butyl 3-amino-4-hydroxypyrrolidine-1-carboxylate hydrochloride (139 mg, 0.583 mmol), followed by diisopropyl ethylamine (135 μL, 0.777 mmol). The reaction mixture was stirred at rt overnight. After diluting with EtOAc and washed with water, the organic layer was washed with brine and dried over Na₂SO₄ before concentrating to an oily residue.

### Step 2:

The residue from the previous step was dissolved in DCM (4 mL) and treated with 1 mL of TFA. The reaction mixture was stirred at rt for 2 d. After concentrating the reaction mixture, the residue was redissolved in EtOAc and washed with sat. NaHCO₃ soln. The organic layer was dried over Na₂SO₄ and concentrated before purification by prep HPLC to give compound 396 as a white solid (34 mg, 21%).

¹H NMR (400 MHz, DMSO-d6) δ 9.30-9.13 (m, 1H), 8.96-8.83 (m, 1H), 8.23 (d, J=4.8 Hz, 1H), 7.91 (s, 1H), 7.58 (s, 1H), 7.39 (s, 1H), 5.92 (s, 1H), 4.45 (s, 1H), 4.07 (s, 1H), 3.53 (s, 2H), 3.15 (s, 1H), 2.68 (s, 3H), 2.14 (s, 1H), 1.62 (s, 9H), 1.04 (d, J=6.3 Hz, 2H), 0.84 (d, J=5.3 Hz, 2H); ¹⁹F NMR (376.1 MHz) δ −74.09 (TFA salt); MS [M+H]⁺=408.3; LC/MS RT=2.08 min.

### Preparation of Compounds 397-400

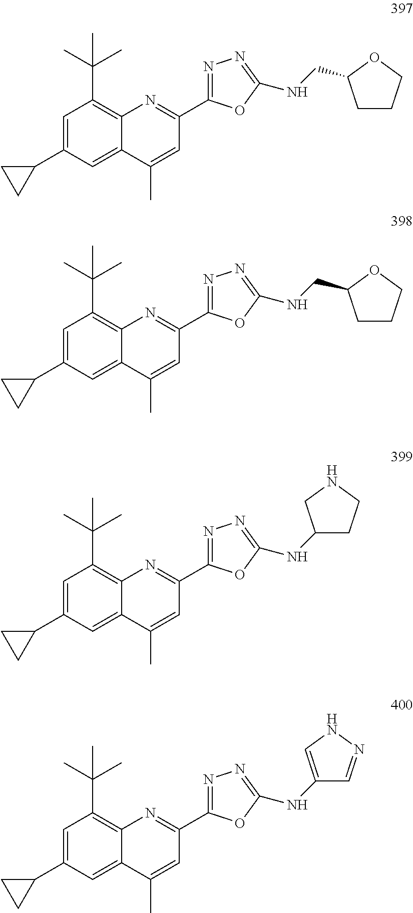

The compounds in the example were made according to procedures described in example 31.

397: ¹H NMR (400 MHz, DMSO-d6) δ 8.12 (s, 1H), 7.86 (s, 1H), 7.56 (s, 1H), 7.38 (s, 1H), 4.07-3.98 (m, 1H), 3.79-3.71 (m, 1H), 3.61 (dd, J=14.3, 7.3 Hz, 1H), 3.28 (t, J=5.9 Hz, 2H), 2.67 (s, 3H), 2.13 (s, 1H), 1.91 (d, J=12.0 Hz, 1H), 1.82 (s, 2H), 1.59 (d, J=24.4 Hz, 11H), 1.03 (d, J=8.3 Hz, 2H), 0.84 (d, J=6.9 Hz, 2H); ¹⁹F NMR (376.1 MHz) δ −75.10 (TFA salt); MS [M+H]⁺=407.3; LC/MS RT=2.58 min.

398: ¹H NMR (400 MHz, DMSO-d6) δ 8.12 (s, 1H), 7.86 (s, 1H), 7.56 (s, 1H), 7.38 (s, 1H), 4.03 (t, J=6.2 Hz, 1H), 3.74 (t, J=6.8 Hz, 1H), 3.61 (dd, J=14.1, 7.5 Hz, 1H), 3.28 (t, J=5.9 Hz, 2H), 2.67 (s, 3H), 2.13 (s, 1H), 1.91 (d, J=12.0 Hz, 1H), 1.82 (s, 2H), 1.62 (s, 10H), 1.03 (d, J=6.3 Hz, 2H), 0.84 (d, J=6.0 Hz, 2H); ¹⁹F NMR (376.1 MHz) δ −75.20 (TFA salt); MS [M+H]⁺=407.3; LC/MS RT=2.59 min.

399: ¹H NMR (400 MHz, DMSO-d6) δ 8.02 (s, 2H), 7.89 (s, 1H), 7.57 (s, 1H), 7.39 (s, 1H), 3.99 (s, 1H), 3.80 (s, 1H), 3.66 (d, J=33.5 Hz, 4H), 2.68 (s, 3H), 2.37-2.26 (m, 1H), 2.14 (s, 2H), 1.63 (s, 9H), 1.04 (d, J=6.2 Hz, 2H), 0.84 (d, J=6.0 Hz, 2H); ¹⁹F NMR (376.1 MHz) δ −74.47 (TFA salt); MS [M+H]⁺=392.3; LC/MS RT=2.12 min.

400: ¹H NMR (400 MHz, DMSO-d6) δ 10.45 (s, 1H), 7.92 (s, 1H), 7.69 (s, 2H), 7.58 (s, 1H), 7.40 (s, 1H), 2.69 (s, 3H), 2.14 (s, 1H), 1.66 (s, 9H), 1.04 (d, J=8.6 Hz, 2H), 0.85 (d, J=4.8 Hz, 2H); ¹⁹F NMR (376.1 MHz) δ −74.97 (TFA salt); MS [M+H]⁺=389.3; LC/MS RT=2.41 min.

### Compound 401

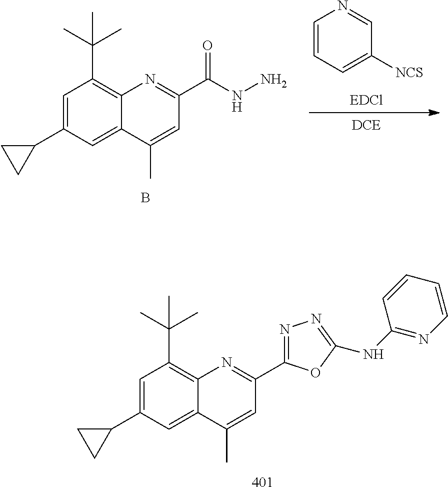

Compound B from example 31 (80 mg, 0.269 mmol), suspended in DCE (2 mL), was treated with 3-pyridyl isothiocyanate (36 mg, 0.269 mmol). The reaction mixture was stirred at 60° C. for 2 h. It was then cooled to rt and EDCl (155 mg, 0.808 mmol) was then added. The reaction mixture was heated at 60° C. for 30 min. It was concentrated and the residue was suspended in DMF and filtered through a syringe filter before purification by prep HPLC to give compound 401 as a white solid (15 mg, 14%).

¹H NMR (400 MHz, DMSO-d6) δ 11.19 (s, 1H), 8.87 (s, 1H), 8.29 (s, 1H), 8.24-8.17 (m, 1H), 7.97 (s, 1H), 7.60 (s, 1H), 7.55-7.48 (m, 1H), 7.41 (s, 1H), 3.56 (s, 5H), 2.71 (s, 3H), 2.15 (s, 1H), 1.66 (s, 9H), 1.06 (s, 2H), 0.86 (s, 2H); ¹⁹F NMR (376.1 MHz) δ −74.78 (TFA salt); MS [M+H]⁺=400.3; LC/MS RT=2.54 min.

### Compound 402

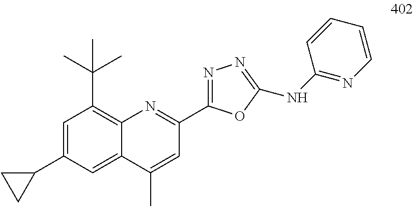

The compounds in the example were made according to procedures described previously.

¹H NMR (400 MHz, DMSO-d6) δ 8.28 (s, 1H), 7.95 (s, 1H), 7.85 (s, 2H), 7.60 (s, 1H), 7.41 (s, 1H), 7.02 (s, 1H), 2.71 (s, 3H), 2.15 (s, 1H), 1.65 (s, 9H), 1.05 (d, J=8.1 Hz, 2H), 0.86 (d, J=6.6 Hz, 2H); ¹⁹F NMR (376.1 MHz) δ −75.11 (TFA salt); MS [M+H]⁺=400.3; LC/MS RT=2.61 min.

### Compounds 403

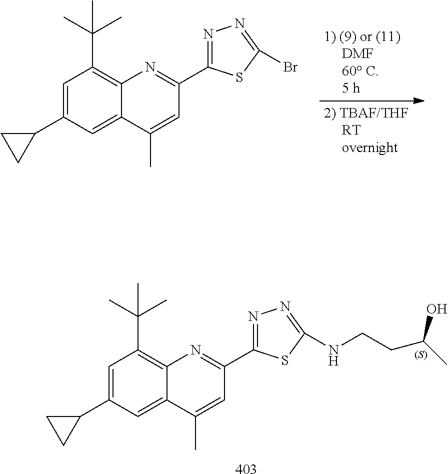

¹H-NMR (400 MHz, CDCl₃) δ 11.35 (m, 1H), 7.87 (s, 1H), 7.53 (s, 1H), 7.43 (s, 1H), 4.03 (m, 1H), 3.66-3.57 (m, 2H), 2.72 (s, 3H), 2.11 (m, 2H), 1.99 (m, 1H), 1.86 (m, 1H), 1.65 (s, 9H), 1.30 (d, 3H), 1.09 (m, 2H), 0.85 (m, 2H); ¹⁹F NMR (376.1 MHz) δ −76.5 (s); MS [M+H]⁺=411.3

### Compound 404

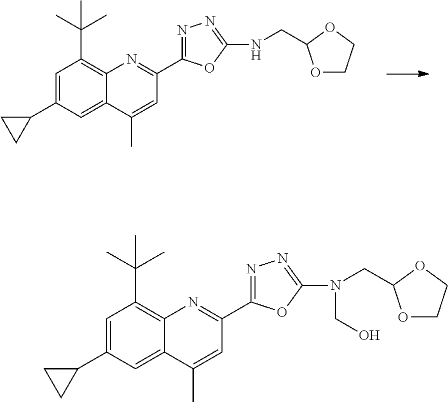

Compound 283 (25 mg, 0.061 mmol) was taken up in 10 mL 36.5% formaldehyde and heated at 60 C for 1 h. The reaction was diluted with EtOAc and washed with water three times. The organic layer was dried with sodium sulfate and concentrated to provide the desired product (27.6 mg, 99% yield) as a white solid. ¹H-NMR (400 MHz, DMSO) δ 7.89 (s, 1H), 7.58 (s, 1H), 7.38 (s, 1H), 6.26 (t, J=7 Hz, 1H), 5.15 (t, J=4 Hz, 1H), 4.95 (d, J=7 Hz, 2H), 4.76 (d J=6 Hz, 1H) 3.92 (m, 2H), 3.80 (m, 2H), 2.67 (s, 3H), 2.14 (m, 1H), 1.03 (m, 2H), 0.84 (m, 2H); MS [M−H]⁺=439.16.

### Compound 405

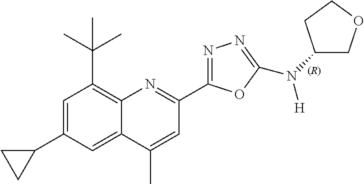

¹H NMR (400 MHz, dmso) δ 8.25 (d, J=5.9 Hz, 1H), 7.87 (s, 1H), 7.56 (s, 1H), 7.37 (s, 1H), 4.20 (s, 1H), 3.89-3.77 (m, 2H), 3.71 (ddd, J=15.9, 8.5, 4.4 Hz, 2H), 2.67 (s, 3H), 2.24-2.07 (m, 2H), 1.95 (d, J=3.8 Hz, 1H), 1.62 (s, 9H), 1.09-0.97 (m, 2H), 0.88-0.79 (m, 2H)

### Compound 406

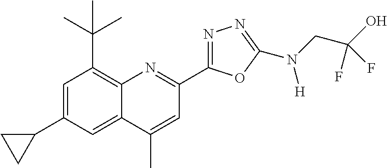

This compound was prepared by coupling of the appropriate isothiocyanate and hydrazide followed by EDCl cyclization to the 1,3,4-oxadiazole. Deprotection was accomplished via TFA/pTosH.

¹H NMR (400 MHz, dmso) δ 8.46 (t, J=6.4 Hz, 1H), 7.88 (s, 1H), 7.57 (s, 1H), 7.38 (s, 1H), 5.57 (t, J=6.4 Hz, 1H), 3.89-3.53 (m, 4H), 2.68 (s, 3H), 2.20-2.03 (m, 1H), 1.62 (s, 9H), 1.09-0.97 (m, 2H), 0.87-0.67 (m, 2H); MS [M+H]⁺=417.27.

### Compound 407

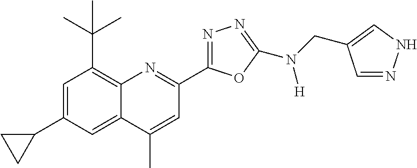

¹H NMR (400 MHz, dmso) δ 8.38 (s, 1H), 7.84 (s, 1H), 7.53 (s, 2H), 7.34 (s, 1H), 6.20 (s, 1H), 4.40 (d, J=5.7 Hz, 2H), 2.64 (s, 3H), 2.10 (s, 1H), 1.58 (s, 10H), 1.12-0.90 (m, 2H), 0.81 (d, J=6.9 Hz, 2H); MS [M+H]⁺=417.27

### Compound 408

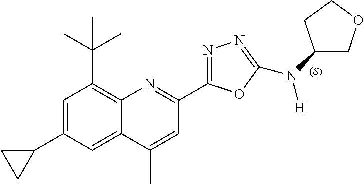

¹H NMR (400 MHz, dmso) δ 8.25 (d, J=5.7 Hz, 1H), 7.88 (s, 1H), 7.57 (s, 1H), 7.38 (s, 1H), 4.20 (s, 1H), 3.93-3.77 (m, 2H), 3.71 (ddd, J=16.0, 8.6, 4.3 Hz, 2H), 2.68 (s, 3H), 2.25-2.01 (m, 2H), 1.96 (s, 1H), 1.62 (s, 9H), 1.03 (dd, J=7.3, 5.1 Hz, 2H), 0.90-0.75 (m, 2H); MS [M+H]⁺=417.27.

### Compound 409

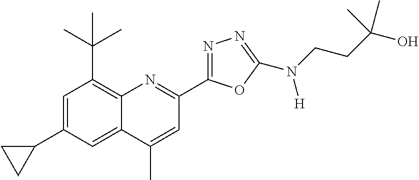

¹H NMR (400 MHz, dmso) δ 7.90-7.83 (m, 2H), 7.56 (s, 1H), 7.37 (s, 1H), 4.34 (s, 1H), 3.34 (dd, J=10.6, 5.4 Hz, 2H), 2.67 (s, 3H), 2.21-2.05 (m, 1H), 1.76-1.64 (m, 2H), 1.61 (s, 9H), 1.12 (s, 6H), 1.03 (td, J=6.3, 4.3 Hz, 2H), 0.88-0.80 (m, 2H); MS [M+H]⁺=417.27.

### Compound 410

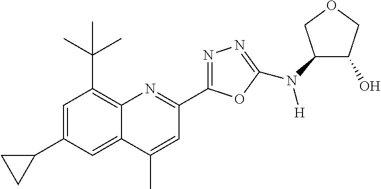

¹H NMR (400 MHz, dmso) δ 8.25 (d, J=5.9 Hz, 1H), 7.88 (s, 1H), 7.56 (s, 1H), 7.37 (s, 1H), 4.27 (s, 1H), 3.94 (ddd, J=13.5, 9.2, 4.8 Hz, 3H), 3.73 (d, J=8.8 Hz, 1H), 3.53 (d, J=9.5 Hz, 1H), 2.67 (s, 3H), 2.19-2.06 (m, 1H), 1.61 (s, 8H), 1.04 (t, J=7.3 Hz, 2H), 0.84 (d, J=5.3 Hz, 2H); MS [M+H]⁺=409.27.

### Compound 411

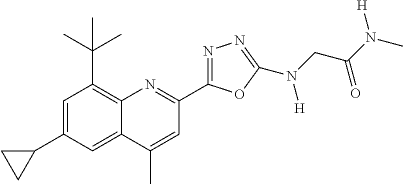

¹H NMR (400 MHz, dmso) δ 8.27 (t, J=6.3 Hz, 1H), 7.97 (d, J=4.5 Hz, 1H), 7.88 (s, 1H), 7.57 (d, J=1.5 Hz, 1H), 7.38 (d, J=1.6 Hz, 1H), 3.82 (d, J=6.2 Hz, 2H), 2.68 (s, 3H), 2.58 (d, J=4.6 Hz, 3H), 1.62 (s, 9H), 1.07-0.97 (m, 2H), 0.88-0.79 (m, 2H;

¹⁹F NMR (376 MHz, dmso) δ −75.18; MS [M+H]⁺=394.32.

### Compound 410

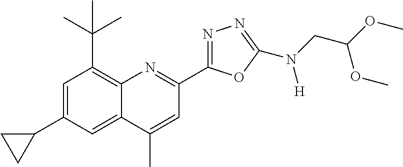

¹H NMR (400 MHz, dmso) δ 8.13 (d, J=6.1 Hz, 1H), 7.87 (s, 1H), 7.57 (d, J=1.6 Hz, 1H), 7.38 (d, J=1.7 Hz, 1H), 4.57 (t, J=5.5 Hz, 1H), 3.34 (t, J=5.8 Hz, 2H), 2.67 (s, 3H), 2.17-2.04 (m, 1H), 1.62 (s, 9H), 1.06-0.99 (m, 2H), 0.87-0.81 (m, 2H); MS [M+H]⁺=411.41.

### Compound 411

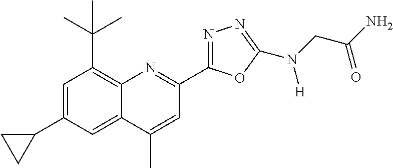

¹H NMR (400 MHz, dmso) δ 8.18 (t, J=6.3 Hz, 1H), 7.87 (s, 1H), 7.57 (s, 1H), 7.49 (s, 1H), 7.38 (s, 1H), 7.09 (s, 1H), 3.80 (d, J=6.3 Hz, 2H), 2.68 (s, 3H), 2.18-2.08 (m, 1H), 1.62 (s, 9H), 1.10-0.99 (m, 2H), 0.88-0.79 (m, 2H); ¹⁹F NMR (376 MHz, dmso) δ −75.02;

### Compound 412

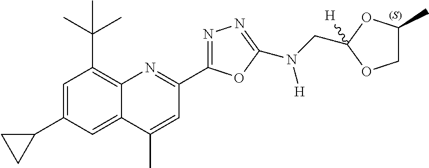

NMR (400 MHz, dmso) δ 8.19 (q, J=6.0 Hz, 1H), 7.86 (s, 1H), 7.57 (d, J=1.5 Hz, 1H), 7.38 (s, 1H), 5.16 (t, J=4.5 Hz, 0.5H), 5.04 (t, J=4.3 Hz, 0.5H), 4.22 (dd, J=12.8, 6.1 Hz, 0.5H), 4.10 (dt, J=8.0, 6.0 Hz, 1H), 3.98-3.86 (m, 0.5H), 3.47-3.15 (m, 3H), 2.67 (s, 3H), 2.19-2.05 (m, 1H), 1.61 (s, 9H), 1.16 (dd, J=13.7, 6.1 Hz, 3H), 1.07-0.97 (m, 2H), 0.89-0.79 (m, 2H); ¹⁹F NMR (376 MHz, dmso) δ −75.32; MS [M+H]⁺=423.43.

### Compound 413

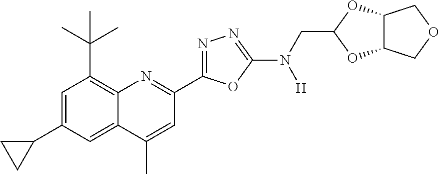

¹H NMR (400 MHz, dmso) δ 8.22 (t, J=6.0 Hz, 1H), 7.86 (s, 1H), 7.56 (s, 1H), 7.38 (s, 1H), 5.24 (s, 0.25H), 5.06 (t, J=4.6 Hz, 0.75H), 4.80 (s, 0.5H), 4.71 (s, 1.5H), 3.88 (dd, J=11.3, 3.6 Hz, 2H), 3.37 (ddd, J=23.8, 14.3, 7.9 Hz, 4H), 2.67 (s, 3H), 2.13 (td, J=8.4, 4.2 Hz, 1H), 1.61 (s, 9H), 1.08-0.99 (m, 2H), 0.87-0.79 (m, 2H); ¹⁹F NMR (376 MHz, dmso) δ −75.23; MS [M+H]⁺=451.47

### Compound 414

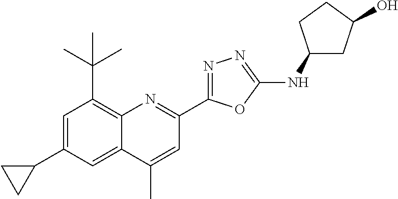

¹H NMR (400 MHz, dmso) δ 8.00 (d, J=6.7 Hz, 1H), 7.86 (s, 1H), 7.56 (s, 1H), 7.38 (s, 1H), 4.14-4.03 (m, 1H), 3.88 (dd, J=14.3, 6.9 Hz, 2H), 2.67 (s, 3H), 2.32-2.18 (m, 1H), 2.17-2.07 (m, 1H), 1.94 (d, J=5.3 Hz, 1H), 1.81-1.64 (m, 1H), 1.62 (s, 9H), 1.55-1.45 (m, 1H), 1.09-0.98 (m, 2H), 0.88-0.78 (m, 2H); ¹⁹F NMR (376 MHz, dmso) δ −74.64; MS [M+H]⁺=407.29.

### Compound 415

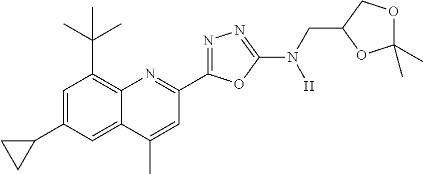

¹H NMR (400 MHz, dmso) δ 8.14 (d, J=5.9 Hz, 1H), 7.87 (s, 1H), 7.56 (s, 1H), 7.38 (s, 1H), 4.28 (t, J=6.0 Hz, 1H), 4.06-3.97 (m, 1H), 3.72 (dd, J=8.3, 5.7 Hz, 1H), 3.35 (t, J=6.1 Hz, 2H), 2.67 (s, 3H), 2.13 (s, 1H), 1.62 (s, 9H), 1.32 (s, 3H), 1.24 (s, 3H), 1.08-1.00 (m, 2H), 0.84 (d, J=6.8 Hz, 2H); MS [M+H]⁺=437.16.

### Compound 416

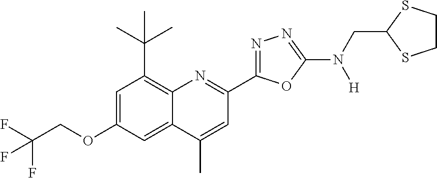

¹H NMR (400 MHz, dmso) δ 8.45 (t, J=5.9 Hz, 1H), 7.87 (s, 1H), 7.57 (s, 1H), 7.44-7.26 (m, 2H), 4.74 (t, J=7.2 Hz, 1H), 3.52-3.33 (m, 2H), 3.21 (s, 3H), 2.13 (td, J=8.4, 4.3 Hz, 1H), 1.62 (s, 9H), 1.08-0.96 (m, 2H), 0.87-0.78 (m, 2H); ¹⁹F NMR (376 MHz, dmso) δ −73.57, −73.59; MS [M+H]⁺=441.16

### Compounds 417-419

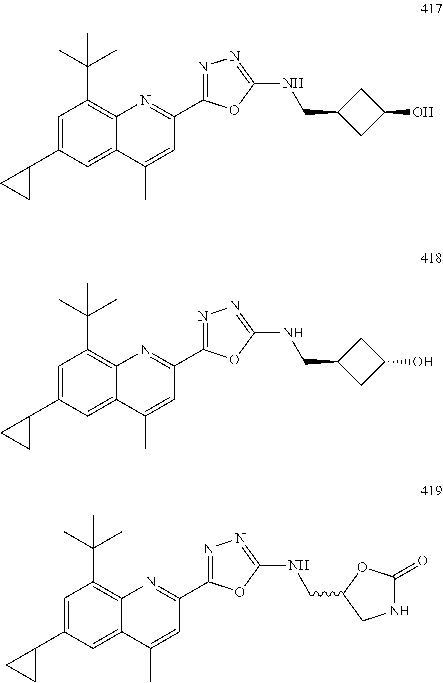

Compound 417: ¹H-NMR (400 MHz, DMSO-d₆) δ 8.01 (m, 1H), 7.86 (s, 1H), 7.56 (s, 1H), 7.37 (s, 1H), 3.87 (m, 1H), 3.24 (m, 2H), 2.67 (s, 3H), 2.26 (m, 2H), 2.13 (m, 1H), 2.00 (m, 1H), 1.62 (s, 9H), 1.55 (m, 2H), 1.03 (m, 2H), 0.84 (m, 2H). ¹⁹F NMR (376.1 MHz) δ −75.20 (s); MS [M+H]⁺=407.3

Compound 418: ¹H-NMR (400 MHz, DMSO-d₆) δ 8.05 (m, 1H), 7.86 (s, 1H), 7.56 (s, 1H), 7.37 (s, 1H), 4.20 (m, 1H), 3.28 (m, 2H), 2.67 (s, 3H), 2.38 (m, 1H), 2.13 (m, 1H), 2.00 (m, 2H), 1.90 (m, 2H), 1.61 (s, 9H), 1.02 (m, 2H), 0.84 (m, 2H). ¹⁹F NMR (376.1 MHz) δ −75.27 (s); MS [M+H]⁺=407.3

Compound 419: ¹H-NMR (400 MHz, DMSO-d₆) δ 8.30 (m, 1H), 7.87 (s, 1H), 7.56 (m, 1H), 7.56 (s, 1H), 7.38 (s, 1H), 4.77 (m, 1H), 3.56 (m, 1H), 3.49 (m, 2H), 3.27 (m, 2H), 2.67 (s, 3H), 2.13 (m, 1H), 1.61 (s, 9H), 1.04 (m, 2H), 0.84 (m, 2H). ¹⁹F NMR (376.1 MHz) δ −75.04 (s); MS [M+H]⁺=422.3

### Compounds 420-422

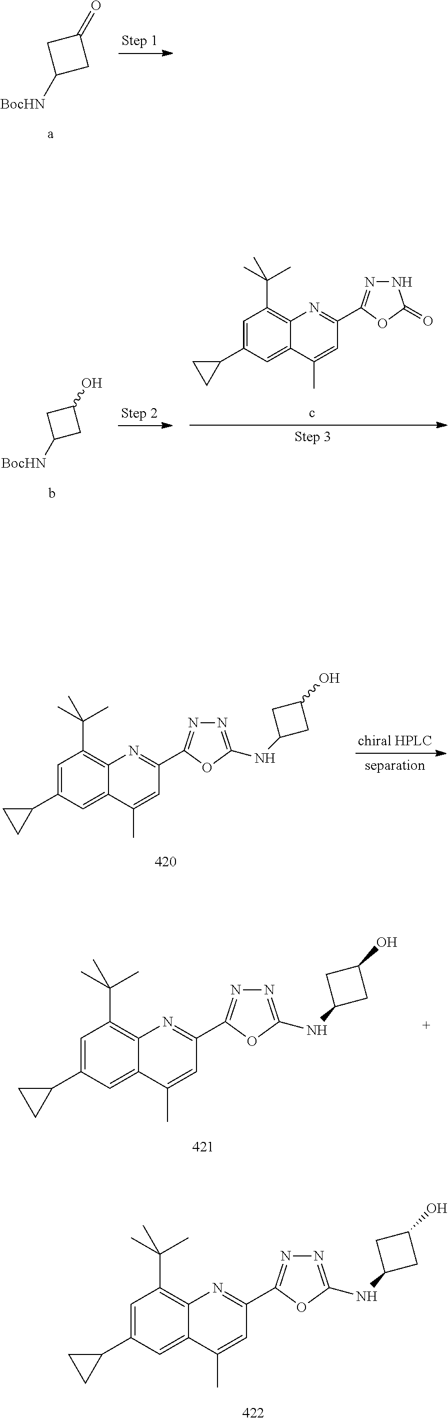

### Step 1

A solution of compound a (2.00 g, 10.80 mmol) in ethanol (20 mL) was stirred at 0° C. as NaBH₄ (210 mg, 5.55 mmol) was added. After 2 h at 0° C. The solution was concentrated and the residue was dissolved in CH₂Cl₂ and washed with aq. NaHCO₃ and water (1:1). After the aq. fraction was extracted with CH₂Cl₂ (×2), the organic fractions were washed with water (×1), combined, dried (Na₂SO₄), and concentrated to obtain crude compound b (2.13 g). ¹H-NMR (400 MHz, CDCl₃) δ 4.65 (br, 1H), 4.47 (m, 0.3H), 4.22 (br, 0.3H), 4.02 (quintet, J=−7.1 Hz, 0.7H), 3.65 (br, 0.7H), 2.76 (m, 1.4H), 2.30 (m, 0.6H), 2.22 (m, 0.6H), 1.87 (br, 1H), 1.79 (m, 1.4H), 1.43 (s, 9H)

### Step 2 and 3

A solution of crude compound b (351 mg, 1.88 mmol) in CH₂Cl₂ (5 mL) and 4 M HCl in dioxane (5 mL) was stirred at it for 1 h. The mixture was concentrated and dried in vacuum.

Compound 420 (213 mg, 59%, ˜7:3 mixture of cis and trans isomers) was prepared from compound c (300 mg, 0.93 mmol) in a manner similar to that described previously.

¹H-NMR (400 MHz, CDCl₃) δ 7.96 (s, 1H), 7.51 (s, 1H), 7.41 (s, 1H), 7.26 (s, 1H), 6.07 (d, J=6.8 Hz, 0.7H), 5.92 (d, J=4.4 Hz, 0.3H), 4.67 (quintet, J=5.9 Hz, 0.3H), 4.47 (sixtet, J=˜5 Hz, 0.3H), 4.17 (quintet, J=6.9 Hz, 0.7H), 3.90 (sixtet, J=7.5 Hz, 0.7H), 3.00 (m, 1.4H), 2.69 (s, 3H), 2.53 (m, 1.2H), 2.21 (m, 1.4H), 2.09 (m, 1H), 1.68 (s, 2.7H), 1.67 (s, 6.3H), 1.08 (m, 2H), 0.85 (m, 2H); MS [M+H]⁺=393.3

Two isomers were separated by preparative chiral HPLC.

Compound 421: (14.3 mg): ¹H-NMR (400 MHz, CDCl₃) δ 7.96 (s, 1H), 7.51 (s, 1H), 7.41 (s, 1H), 7.26 (s, 1H), 6.07 (d, J=6.8 Hz, 1H), 4.17 (quintet, J=6.9 Hz, 1H), 3.90 (sixtet, J=7.5 Hz, 1H), 3.00 (m, 2H), 2.69 (s, 3H), 2.21 (m, 2H), 2.09 (m, 1H), 1.67 (s, 9H), 1.08 (m, 2H), 0.85 (m, 2H); MS [M+H]⁺=393.3

Compound 421: (5.8 mg): ¹H-NMR (400 MHz, CDCl₃) δ 7.96 (s, 1H), 7.51 (s, 1H), 7.41 (s, 1H), 7.26 (s, 1H), 5.92 (d, J=4.4 Hz, 1H), 4.67 (quintet, J=5.9 Hz, 1H), 4.47 (sixtet, J=˜5 Hz, 1H), 2.69 (s, 3H), 2.53 (m, 4H), 2.09 (m, 1H), 1.68 (s, 9H), 1.08 (m, 2H), 0.85 (m, 2H); MS [M+H]⁺=393.3

### Compound 423

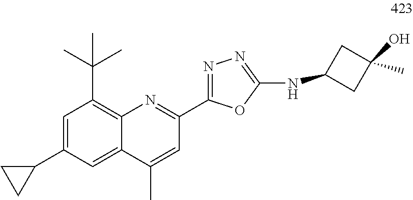

Compound 423 was prepared in the manners similar to compounds 421 and 422, starting from starting material a for compound 420. Desired isomer 423 was obtained by silica gel chromatography.

¹H-NMR (400 MHz, CH₃OH-d₄) δ 7.98 (s, 1H), 7.63 (s, 1H), 7.48 (s, 1H), 3.86 (m, 1H), 2.78 (s, 3H), 2.62 (m, 2H), 2.24 (m, 3H), 1.77 (s, 9H), 1.43 (s, 3H), 1.18 (m, 2H), 0.89 (m, 2H); MS [M+H]⁺=407.

### Example 43

### Compounds 424-431

Compounds 424-431 were prepared from compound 225 in the manner similar to compound 226 in example 26.

### Compound 424

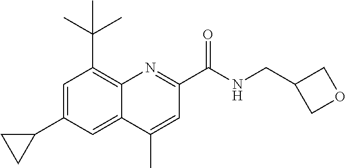

¹H-NMR (400 MHz, CDCl₃) δ 8.3 (t, NH), 8.04 (s, 1H), 7.53 (d, 1H), 7.41 (d, 1H), 4.84 (m, 2H), 4.54 (m, 2H), 3.82 (t, 2H), 3.34 (m, 1H), 2.71 (s, 3H), 2.1 (m, 1H), 1.65 (s, 9H), 1.07 (m, 2H), 0.84 (m, 2H).

### Compound 425

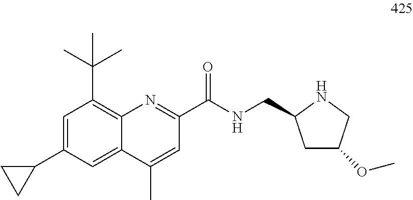

¹H-NMR (400 MHz, CH₃OH-d₄) δ 8.58 (m, 1H), 8.02 (s, 1H), 7.68 (s, 1H), 7.52 (s, 1H), 4.18 (m, 1H), 4.06 (m, 1H), 3.86 (m, 2H), 3.52 (m, 1H), 3.40 (m, 1H), 3.34 (s, 3H), 2.78 (s, 3H), 2.42 (m, 1H), 2.18 (m, 1H), 1.96 (m, 1H), 1.62 (s, 9H), 1.18 (m, 2H), 0.91 (m, 2H); MS [M+H]⁺=396.

### Compound 426

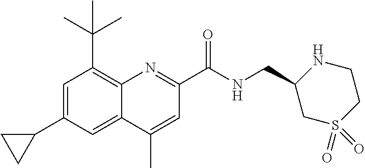

¹H-NMR (400 MHz, DMSO-d6) δ 7.95 (s, 1H), 7.63 (d, 1H), 7.47 (d, 1H), 3.72 (m, 1H), 3.57 (m, 6H), 3.2 (m, 3H), 2.71 (s, 3H), 2.12 (m, 1H), 1.68 (s, 9H), 1.09 (m, 2H), 0.84 (m, 2H).

MS [M+H]⁺=430.22

### Compound 427

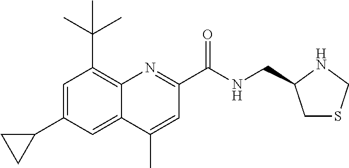

¹H-NMR (400 MHz, DMSO-d6) δ 7.94 (s, 1H), 7.63 (s, 1H), 7.46 (s, 1H), 3.76 (m, 1H), 3.6 (m, 1H), 3.4 (dd, 1H), 3.29 (s, 2H), 3.01 (m, 1H), 2.72 (s, 3H), 2.68 (m, 1H), 2.13 (m, 1H), 1.67 (s, 9H), 1.08 (m, 2H), 0.84 (m, 2H).

MS [M+H]⁺=384.16

### Compound 428

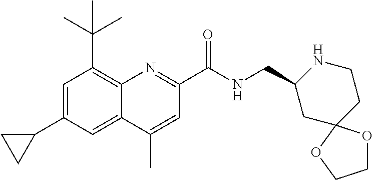

¹H-NMR (400 MHz, CDCl3) δ 8.44 (m, 1H), 8.01 (s, 1H), 7.51 (s, 1H), 7.4 (d, 1H), 3.56 (m, 2H), 3.17 (m, 2H), 2.91 (m, 2H), 2.67 (s, 3H), 2.06 (m, 1H), 1.65 (s, 9H), 1.59 (m, 4H), 1.06 (m, 2H), 0.84 (m, 2H).

MS [M+H]⁺=438.23

### Compound 429

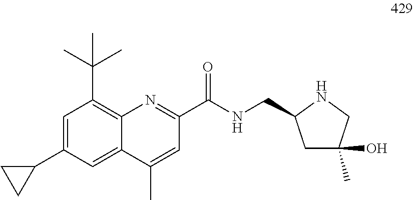

¹H-NMR (400 MHz, CH₃OH-d₄) δ 8.02 (s, 1H), 7.66 (s, 1H), 7.52 (s, 1H), 4.18 (m, 1H), 3.93 (m, 2H), 3.30-3.16 (m, 2H), 2.78 (s, 3H), 2.30 (m, 1H), 2.20 (m, 1H), 1.98 (m, 1H), 1.73 (s, 9H), 1.43 (3, 3H), 1.18 (m, 2H), 0.91 (m, 2H); MS [M+H]⁺=396.

### Compound 430

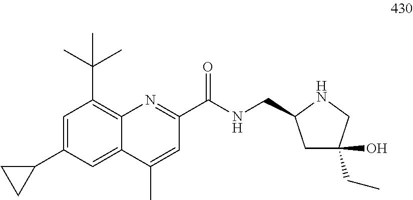

¹H-NMR (400 MHz, CH₃OH-d₄) δ 7.96 (s, 1H), 7.61 (s, 1H), 7.47 (s, 1H), 4.18 (m, 1H), 3.93 (m, 2H), 3.29 (m, 2H), 3.11 (m, 1H), 2.69 (s, 3H), 2.27 (m, 1H), 2.21 (m, 1H), 1.98 (m, 1H), 1.73 (m, 2H), 1.67 (s, 9H), 1.10 (m, 2H), 1.02 (m, 3H), 0.84 (m, 2H); MS [M+H]⁺=410.

### Compound 431

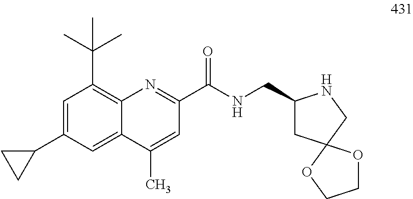

¹H-NMR (400 MHz, CD₃OD₃) δ 7.94 (s, 1H), 7.61 (d, 1H), 7.46 (d, 1H), 4.83-3.85 (m, 3H), 3.60-3.54 (m, 2H), 3.29 (m, 1H), 3.05-2.91 (dd, 2H), 2.70 (s, 3H), 2.12 (m, 2H), 1.80 (m, 1H), 1.67 (s, 9H), 1.09 (m, 2H), 0.83 (m, 2H); MS [M+H]⁺=424.50

### Example 44

### Compound 432

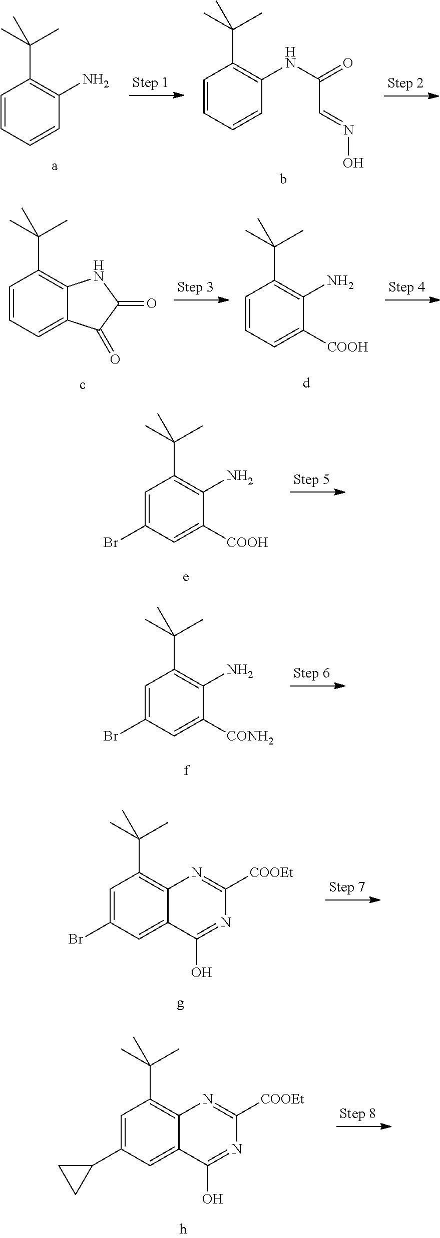

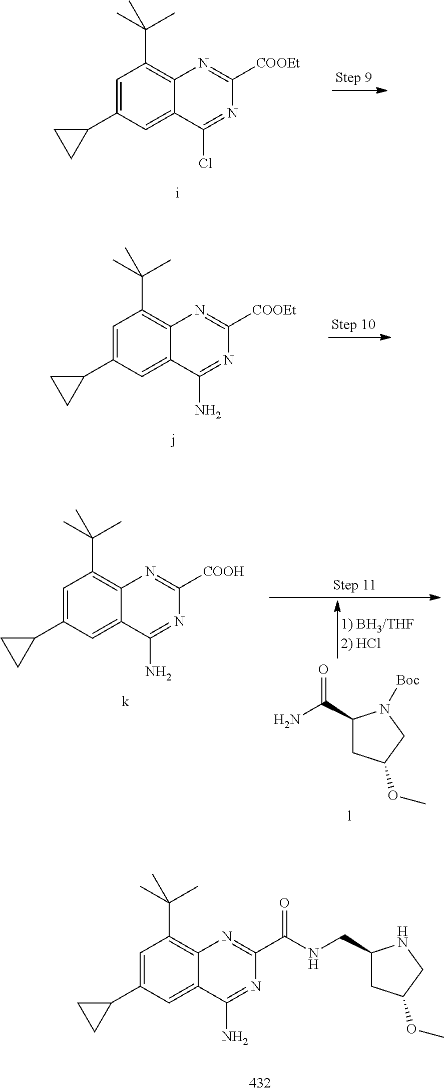

### Step 1 and 2

A mixture of compound a (26.91 g, 0.18 mol), Na₂SO₄ (222.1 g, 1.56 mol), and chloral hydrate (36.1 g, 0.218 mol) in H₂O (1.2 L) was stirred at rt as c. HCl (17 mL) in H₂O (200 mL) followed by H₂NOH—HCl (42.05 g, 0.605 g) in H₂O (100 mL) were added. The resulting mixture was heated at 105° C. bath. After the reflux began, the mixture was stirred 0.5 h at the bath and then slowly cooled to rt. The sticky brown solids were collected, washed with H₂O, and dried before the next step. MS [M+H]⁺=220.9

A flask containing H₂SO₄ (100 mL) was stirred at 85° C. as the solids obtained above was added over 3 min. After the mixture was stirred for 15 min, it was poured to ice (1-1.5 Kg). The precipitate was filtered and the filter cake was stirred with H₂O before filtration. After the filter cake was dissolved in CH₂Cl₂ (500 mL) and refluxed for 30 min, the solution with some black tar was dried with MgSO4 and the resulting solution was concentrated with silica gel. The adsorbed crude product was purified by combiflash using hexanes and dichloromethane to obtain compound c (7.00 g). MS [M+H]⁺=204.1

### Step 3

A suspension of compound c (6.14 g, 30.2 mmol) in 10% aq. NaOH (24 mL) was stirred at 80° C. bath as 30% H₂O₂ in H₂O (7 mL, 68.5 mmol) was added dropwise over 1 h. After addition, the mixture was heated for 30 min and cooled to rt. To the mixture was added activated charcoal (0.5 g) and stirred for 30 min at rt before filtration. The filtrate was neutralized with c. HCl. The resulting mixture was stirred in ice bath for 30 min and filtered. The solids collected was washed with water and dried to obtain compound d (4.653 g, 80%). MS [M+H]⁺=194.0

### Step 4

A solution of compound d (4.653 g, 24.1 mmol) in DMF (25 mL) was stirred at 0° C. as a solution of NBS (4.32 g, 24.3 mmol) was added over 20 min. After 1 h at 0° C., the resulting solution was stirred at rt for 20 h. The solution was concentrated to ˜1/3 volume and poured into an ice cold H₂O (500 mL). After the mixture was stirred for 2 h at 0° C., the precipitated solids were filtered and washed with water, and dried in vacuum to obtain 6.50 g (99%) of compound e. MS [M+H]⁺=272.1

### Step 5

A solution of compound e (6.49 g, 23.86 mmol), HOBt (3.55 g, 26.27 mmol), and EDCl (5.26 g, 27.44 mmol) in DMF (100 mL) was stirred at rt for 1 h. After the solution was cooled at 0° C., c. NH₄OH (2 mL) was added and the solution was stirred for 1 h. After 1 h, additional c. NH₄OH (1 mL) was added and the mixture was stirred at rt for 1.5 h. The solution was concentrated, and the residue was dissolved in ethyl acetate and water with some NaHCO₃ before separation of two fractions. After the aqueous fraction was extracted with ethyl acetate, organic fractions were washed with water (×2), combined, dried (Na₂SO₄) and concentrated with silica gel. The adsorbed sample was purified by combiflash using hexanes and ethyl acetate to obtain 5.56 g (86%) of compound f. MS [M+H]⁺=271.0

### Step 6

A solution of compound f (5.56 g, 10.49 mmol) and 2,6-lutibine (5.3 mL, 45.65 mmol) in THF (100 mL) was starred at 0° C. as ethyl chlorooxoacetate (2.6 mL, 23.27 mmol) was added over 5 min. After the mixture was stirred at rt for 30 min, the mixture was refluxed for 2 d. After the mixture was cooled to rt, it was diluted with ethyl acetate (400 mL), water (400 mL), and saturated aq. NaHCO₃ (100 mL), and the insoluble material was filtered and washed with water and ethyl acetate to obtain compound 13 (2.63 g, 36%). Two layers of the filtrate were separated and the organic fraction was washed with water, dried (Na₂SO₄), and concentrated. After the residue was triturated with ethyl acetate (20 mL) and hexanes (20 mL) mixture at 0° C. for 1 h, the solids were filtered, washed with cold ethyl acetate-hexanes (1:1) mixture, and dried to get additional compound g (3.34 g, 46%). MS [M+H]⁺=353.1

### Step 7

A mixture of compound g (1.002 g, 2.84 mmol), Pd(dppf)Cl₂—CH₂Cl₂, (234 mg, 0.287 mmol), K₂CO₃ (1.564 g, 11.32 mmol), and cyclopropylboronic acid hydrate (886 mg, 8.53 mmol) was degassed and dioxane (10 mL) was added. The mixture was refluxed at 110° C. bath for 1 h. The mixture was dissolved in ethyl acetate and water and filtered to remove insoluble materials. After the two layers were separated, aqueous fraction was extracted with ethyl acetate (×1). The organic fractions were washed with water (×1), combined, dried (Na₂SO₄), and concentrated. The residue was purified by combiflash using hexanes-ethyl acetate to obtain a mixture (224 mg, 5:3:2 ratio) of compound h, compound g, and the debrominated product. MS [M+H]⁺=315.17 and 275.1

### Step 8

A suspension of the product mixture of step 7 in CH₂Cl₂ (5 mL) was stirred at rt as oxalyl chloride (0.2 mL, 2.29 mmol) followed by DMF (2 drops) were added. After 1.5 h, additional oxalyl chloride (0.1 mL, 1.15 mmol) and CH₂Cl₂ (5 mL) were added. After 5.25 h, some silica gel was added to the mixture and the resulting slurry was concentrated. The adsorbed product was purified by combiflash using hexanes-ethyl acetate to obtain a mixture (125 mg, 5:2 ration) of the desired compound i with cyclopropyl lacking impurity. MS [M+H]⁺=333.2 and 293.2

### Step 9, 10 and 11

A solution of the product mixture (101 mg, 5:2 ratio) of step 8 in 0.5 M NH₃ in dioxane (5 mL) was heated at 100° C. bath in a pressure tube for 10.5 h. The mixture was concentrated and the residue was dissolved in THF (1 mL), MeOH (1 mL), and 1 N KOH (0.9 mL). After 1.5 h at rt, The solution was acidified using 1 N HCl (1 mL) and concentrated. After the residue was co-evaporated with toluene (×2), the residual crude compound k was used for the next reaction. MS [M+H]⁺=314.2, 274.2

A solution of compound I (171 mg, 0.70 mmol) in THF (2 mL) was stirred at rt as 1.0 M BH₃ in THF (5 mL) was added. The solution was refluxed for 3 h and cooled to rt before MeOH (5 mL) was added cautiously. After the resulting solution was concentrated, the residue was dissolved in MeOH and concentrated again and dried in vacuum. The residue was dissolved in 4 M HCl in dioxane (5 mL) and stirred at rt for 1 h. After the solution was concentrated, the residue was coevaporated with toluene (×2).

The crude compound was dissolved in DMF (5 mL) and transferred to the flask containing the crude diamine-HCl salt and N-methylmorpholine (0.35 mL, 3.18 mmol). The mixture was stirred at 0° C. as HATU (345 mg, 0.91 mmol) was added. After 1 h at rt, the mixture was stored in freezer overnight. The mixture was diluted with ethyl acetate and washed with 5% LiCl solution followed by water. The aqueous solutions were extracted with ethyl acetate (×1). The organic fractions were combined, dried (Na₂SO₄), and concentrated. The residue was purified by preparative HPLC to obtain compound 432 (25 mg).

¹H-NMR (400 MHz, CD₃OD) δ 7.86 (d, J=1.6 Hz, 1H), 7.80 (d, J=1.6 Hz, 1H), 4.18 (t, J=4.0 Hz, 1H), 4.05 (m, J=5.9 Hz, 1H), 3.89 (d, J=16.0 Hz, 1H), 3.84 (d, J=16.0 Hz, 1H), 3.53 (dd, J=12.8 and 4.4 Hz, 1H), 3.42 (d, J=12.8 Hz, 1H), 3.35 (s, 3H), 3.02 (br, 1H), 2.41 (dd, J=14.0 and 6.0 Hz, 1H), 2.15 (m, 1H), 1.91 (m, 1H), 1.59 (s, 9H), 1.16 (m, 2H), 0.90 (m, 2H); MS [M+H]⁺=398.3

### Compound 433

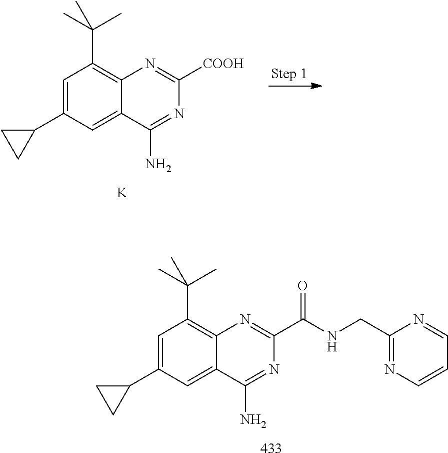

Compound 433 (72 mg) was prepared from compound K in a manner similar to that described previously for compound 432

¹H-NMR (400 MHz, CD₃OD) δ 8.81 (d, J=5.2 Hz, 2H), 7.94 (d, J=2.0 Hz, 1H), 7.88 (d, J=2.0 Hz, 1H), 7.43 (t, J=5.2 Hz, 1H), 4.89 (s, 2H), 2.18 (m, 1H), 1.70 (s, 9H), 1.18 (m, 2H), 0.92 (m, 2H); MS [M+H]⁺=377.3

### Compound 434

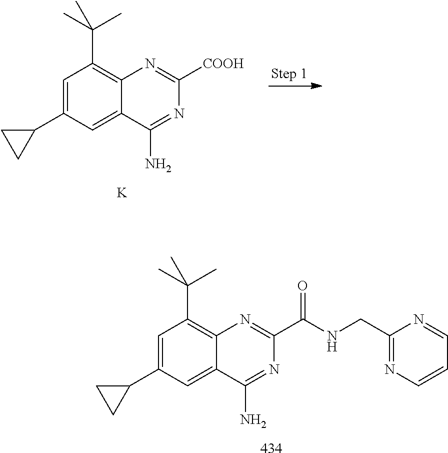

Compound 434 (66 mg) was prepared from compound K in a manner similar to that described previously for compound 432.

¹H-NMR (400 MHz, CD₃OD) δ 8.71 (d, J=1.2 Hz, 1H), 8.61 (dd, J=2.4 and 1.2 Hz, 1H), 8.54 (d, J=2.4 Hz, 1H), 7.94 (d, J=2.0 Hz, 1H), 7.88 (d, J=2.0 Hz, 1H), 4.89 (s, 2H), 2.18 (m, 1H), 1.67 (s, 9H), 1.17 (m, 2H), 0.92 (m, 2H); MS [M+H]⁺=377.3

### Compounds 435-436

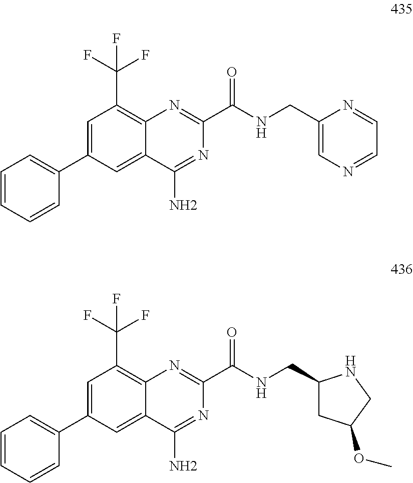

Compounds 435 and 436 were prepared in the manner similar to compound 51 of example 11.

### Compound 435

¹H-NMR (400 MHz, DMSO-d₆) δ 9.04 (m, 1H), 8.95 (s, 1H), 8.66 (s, 1H), 8.60 (d, 1H), 8.54 (d, 1H), 8.44 (s, 1H), 7.92 (s, 1H), 7.90 (s, 1H), 7.55-7.40 (m, 3H), 4.69 (m, 2H), 3.13 (d, 1H); ¹⁹F NMR (376.1 MHz) δ −59.30 (s); MS [M+H]⁺=425.5

### Compound 436

¹H-NMR (400 MHz, CD₃OD₃) δ 8.60 (m, 1H), 8.31 (s, 1H), 7.69 (d, 1H), 7.67 (s, 1H), 7.46-7.35 (m, 3H), 4.12 (m, 1H), 4.02 (m, 1H), 3.72 (m, 2H), 3.46-3.33 (m, 12H), 3.20 (s, 3H), 3.33 (m, 1H), 1.83 (m, 1H); ¹⁹F NMR (376.1 MHz) δ −61.97 (s), −77.66 (s); MS [M+H]⁺=446.2

### Example 45

### Compounds 437-444

Compounds 437-444 were prepared from intermediate 4000 in the manner described below

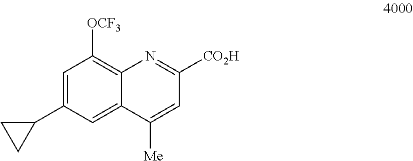

Compound 4000 was used as starting material, itself prepared via a sequence identical to that reported elsewhere in this patent for quinoline compounds with trifluoromethyl and t-butyl groups at the C8 position.

General procedure for amide couplings employing carboxylic acid 4000

100 mg (0.321 mmol) of 4000 was dissolved in 1.6 mL DMF, to which was added 0.244 g HATU (0.642 mmol, 2 eq.), 0.17 mL Heunig's base (0.964 mmol, 3 eq.) and 3 eq. of the corresponding amine. The mixture was stirred at room temperature until LC-MS indicates complete consumption of 4000 (12 hours or less). The mixture was quenched by the addition of 2 mL sat. aq. Ammonium chloride and diluted with 10 mL ethyl acetate and water. The phases are separated and the organic was washed with 5% aqueous LiCl (w/w) and then brine, and concentrated. Preparative HPLC chromatography afforded the pure amide. Product yields ranged from between 30 to 80 percent.

### Compound 437

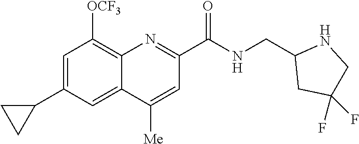

¹H NMR (400 MHz, CDCl₃) δ 8.81 (t, 1H), 8.10 (s, 1H), 7.84 (s, 1H), 7.49 (s, 1H), 4.25 (d, 2H); 1.27-0.89 (m, 5H).

¹⁹F NMR (100 MHz, CDCl₃) δ −58.41, −72.54, −74.41, −76.60 (trifluoroacetate salt) MS [M+H]⁺=430.

### Compound 438

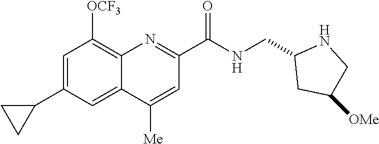

¹H NMR (400 MHz, CDCl₃) δ 9.10; 8.11; 7.93; 7.89; 7.56; 7.52; 1.27-0.86

¹⁹F NMR (100 MHz, CDCl₃) δ −58.38, −72.25, −74.13, −76.30 (trifluoroacetate salt)

MS [M+H]⁺=424.

### Compound 439

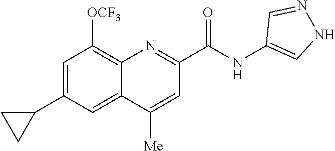

¹H NMR (400 MHz, CDCl₃) δ 9.96 (s, 1H), 8.15 (s, 1H), 7.97 (s, 1H), 7.63 (s, 1H), 7.27 (s, 1H), 7.19 (s, 1H); 2.72 (s, 3H), 1.18-0.79 (m, 5H).

¹⁹F NMR (100 MHz, CDCl₃) δ −57.36

MS [M+H]⁺=377.

### Compound 440

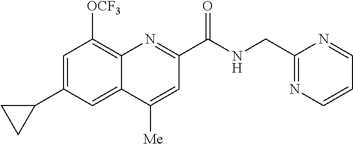

¹H NMR (400 MHz, CDCl₃) δ 9.50, (s, 1H), 8.77 (d, 2H), 8.19 (s, 1H), 7.67 (s, 1H), 7.32 (s, 1H), 4.96 (d, 2H), 2.79 (s, 3H), 0.84-1.17 (m, 5H).

¹⁹F NMR (100 MHz, CDCl₃) δ −57.76

MS [M+H]⁺=403.

### Compound 441

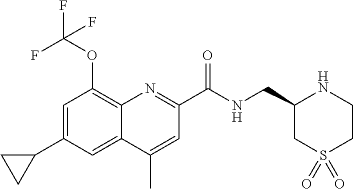

¹H-NMR (400 MHz, MeOD) δ 8.07 (s, 1H), 7.82 (d, 1H), 7.43 (s, 1H), 3.58 (m, 2H), 3.44 (m, 2H), 3.13 (m, 2H), 2.99 (m, 2H), 2.87 (m, 1H), 2.77 (s, 3H), 2.21 (m, 1H), 1.15 (m, 2H), 0.88 (m, 2H)

MS [M+H]⁺=458.05

### Compound 442

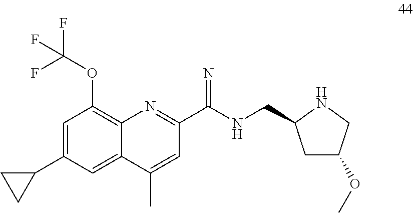

¹H-NMR (400 MHz, CH₃OH-d₄) δ 9.02 (m, 1H), 8.11 (s, 1H), 7.83 (s, 1H), 7.45 (s, 1H), 4.16 (m, 1H), 4.06 (m, 1H), 3.84 (m, 2H), 3.43 (m, 2H), 3.33 (s, 3H), 2.79 (s, 3H), 2.38 (m, 2H), 2.12 (m, 1H), 1.91 (m, 1H), 1.18 (m, 2H), 0.87 (m, 2H); ¹⁹F NMR (400 MHz, CH₃OH-d₄) δ −59.21 (s); MS [M+H]⁺=424.

### Compound 443

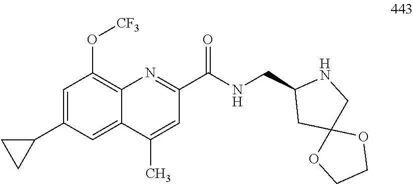

Compound 443 was prepared from an intermediate in example compound 332 and acid 4000 in this example.

¹H-NMR (400 MHz, CD₃OD₃) δ 8.07 (s, 1H), 7.82 (s, 1H), 7.43 (s, 1H), 3.89 (m, 4H), 3.63 (s, 3H), 3.18-2.96 (m, 3H), 2.78 (s, 2H), 2.20 (m, 2H), 1.85 (m, 1H), 1.27 (m, 4H), 1.15 (m, 2H), 0.88 (m, 2H); ¹⁹F NMR (376.1 MHz) δ −59.16 (s); MS [M+H]⁺=452.2

### Compound 444

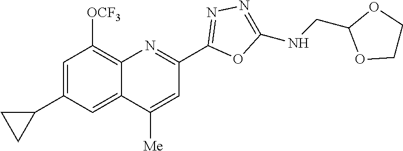

¹H NMR (400 MHz, CDCl₃) δ 8.32 (t, 1H), 8.03 (s, 1H), 7.79 (s, 1H), 7.52 (s, 1H), 5.02 (t, 1H), 3.92-3.80 (m, 5H), 3.09 (m, 2H), 2.73 (s, 3H), 0.88 (comp, 5H).

¹⁹F NMR (100 MHz, CDCl₃) δ −56.57 (s), −74.70 (s).

MS [M+H]⁺=437.

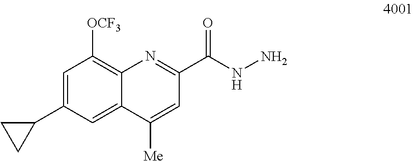

2.93 g BOP reagent (6.95 mmol, 1.25 eq.) was added to a solution of 4000 from this example in 11 mL DMF. 1.23 mL of N-methylmorpholine (11.12 mmol, 2 eq.) was added, followed by 1.10 g tert-butyl hydrazinecarboxylate (8.34 mmol, 1.5 eq.). The mixture was stirred at room temperature for 30 minutes, then 10 mL saturated aq. ammonium chloride was added. The mixture was diluted with 30 mL ethyl acetate and 30 mL water, the phases are separated, and the organics washed with 5% aqueous LiCl (w/w) and then brine, then dried and concentrated. Flash column chromatography (0% EtOAc→100% EtOAc, over 6 column volumes) provided 0.59 g of Boc-protected acyl hydrazide.

This material was dissolved in 1.1 mL of CH₂Cl₂, to which was added 1.1 mL TFA. After 30 minutes, 5 mL ethyl acetate was added, as was 5 mL of 10% sodium citrate. The phases were separated and the organic dried and concentrated to provide 400 mg of 4001.

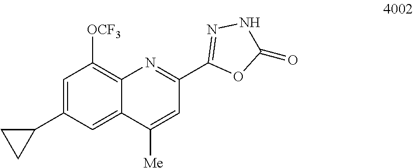

400 mg of 4001 (1.23 mmol) was dissolved in 25 mL of CH₂Cl₂, to which was added 0.52 mL Heunig's base (3.07 mmol, 2.5 eq.) and 0.128 g triphosgene (0.43 mmol, 0.35 eq.). The mixture was stirred until complete consumption of starting material was indicated by LC-MS or cessation of further reactivity. The reaction was diluted with 10 mL DI water, the layers separated, and the organic dried and concentrated to provide 4002, which was carried forward without additional purification.

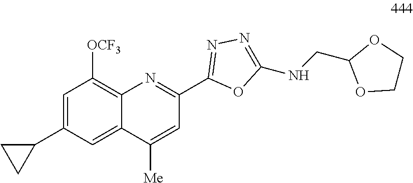

To a solution of 223 mg of 4002 (0.64 mmol) in 6.4 mL CH₂Cl₂ was added 0.22 mL Heunig's base (1.27 mmol, 2 eq.), 0.2 g (1,3-dioxolan-2-yl)methanamine (1.90 mmol, 3 eq.), and 0.29 g BOP reagent (0.64 mmol, 1 eq.). The mixture was stirred overnight, then quenched with 5 mL saturated aqueous ammonium chloride. This mixture was diluted with 15 mL ethyl acetate and 15 mL DI water, the layers separated, and the organic washed with 5% aqueous LiCl (w/w) then brine, then dried and concentrated. Preparative HPLC afforded 7.5 mg of 444, spectral data for which is presented above.

### Compounds 445-448

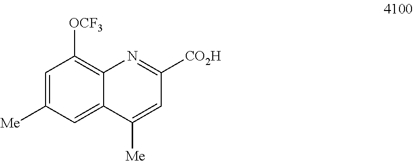

ref Tet Lett 45 (2001) 817-819.

Preparation of intermediate 4100 was based on the lit. and previously described procedures. Coupling of 4100 with appropriate amines yielded compounds 445 to 448.

### Compound 445

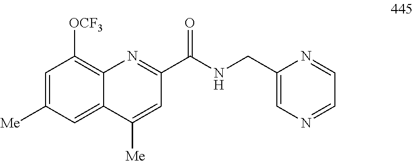

¹H NMR (400 MHz, CDCl₃) δ 9.22 (t, 1H), 8.66-7.76 (m, 6H), 4.76 (d, 2H), 2.76 (s, 3H), 2.57 (s, 3H).

¹⁹F NMR (100 MHz, CDCl₃) δ −56.86.

MS [M+H]⁺=377.

### Compound 446

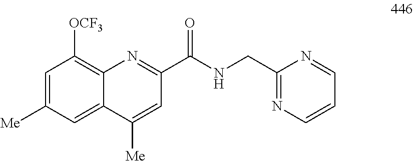

¹H NMR (400 MHz, CDCl₃) δ 9.27 (s, 1H), 8.79-7.43 (m, 6H), 4.79 (s, 2H), 2.77, (s, 3H), 2.48, (s, 3H).

¹⁹F NMR (100 MHz, CDCl₃) δ −56.72, s MS [M+H]⁺=377.

### Compounds 447,447b

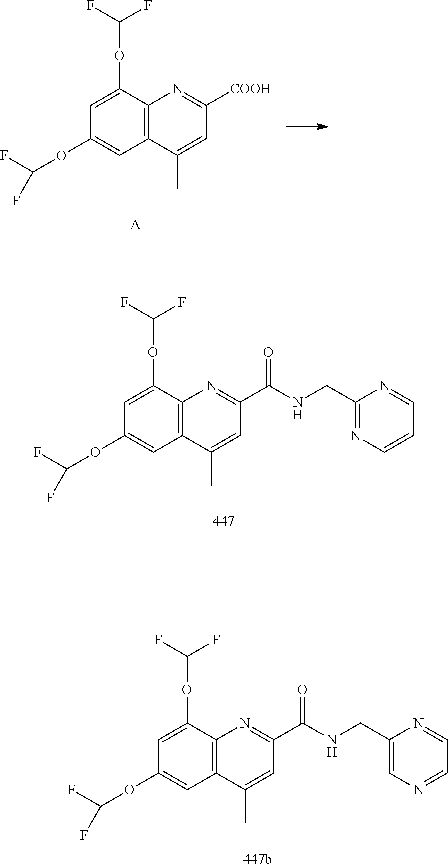

Compounds 447 and 447b were prepared from intermediate A in a similar manner as the preparation of compound 226 from compound 225.

Compound 447: ¹H-NMR (400 MHz, DMSO) δ 7.56 (s, 2H), 7.16 (dd, J=8.6, 5.7 Hz, 3H), 6.51 (s, 2H), 6.24-5.85 (m, 7H), 5.80 (d, J=5.0 Hz, 3H), 3.17 (s, 2H), 1.14 (d, J=6.4 Hz, 3H). MS [M+H]⁺=411.

Compound 448: ¹H-NMR (400 MHz, DMSO) 7.64 (s, 1H), 7.24-6.88 (m, 3H), 6.56 (s, 1H), 6.17 (dd, J=44.7, 29.6 Hz, 2H), 5.99 (s, 1H), 3.19 (s, 2H), 1.19 (s, 3H). MS [M+H]⁺=411.

### Example 46

### Compound 448

### Stage A

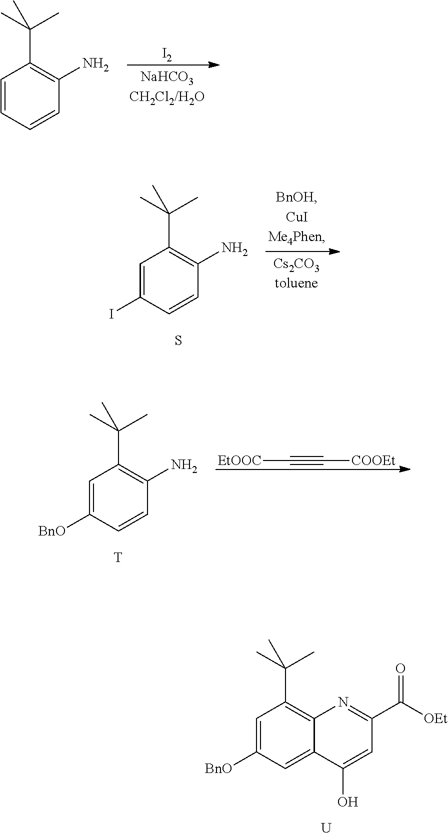

A mixture of 27.80 g (186.3 mmol) of 2-tert-butylaniline and 27.90 g (332 mmol) of sodium bicarbonate in dichloromethane (190 mL) and water (190 mL) was stirred vigorously at 0° C. bath 47.44 g (186.9 mmol) of iodine was added portion wise (every 5 min) over 1 h. After addition, the mixture was stirred for 30 min at 0° C. bath and the mixture was diluted with dichloromethane, water (200 mL each), and some aq. Na₂S₂O₃ solution before two layers were separated. The aqueous fraction was extracted with dichloromethane (100 mL×1) and the two organic fractions were washed with water (×1), combined, dried (Na₂SO₄), and concentrated to dryness to obtain 50.30 g (98%) of the crude iodide S.

A flask containing the crude iodide S (13.185 g, 47.92 mmol), CuI (458 mg, 2.408 mmol), 3,4,7,8-tetramethyl-1,10-phenanthroline (1.134 g, 4.799 mmol), cecium carbonate (18.752 g, 57.55 mmol), and a magnetic stir bar was evacuated and back-filled with argon 3 times. After benzyl alcohol (10.0 mL, 96.54 mmol) and toluene (24 mL) were added to this mixture, the flask was capped tightly and the resulting mixture was stirred at 80° C. for 17 h. The mixture was further stirred at 110° C. for 6 h and cooled to rt before dilution with ethyl acetate. The mixture was filtered through silica gel pad and the silica gel pad was washed with ethyl acetate (total 200 mL of ethyl acetate was used). After the filtrate was concentrated, the residual oil was purified by combiflash (330 g column) using hexane and ethyl acetate to obtain 9.964 g (81%) of T as dark brown solids. Aniline substrate T (10.0 g, 39.2 mmol) was taken up in diphenyl ether (50 mL) and treated with diethyl acetylenedicarboxylate (6.9 mL, 43.1 mmol). The mixture was heated to 60° C. for 1 h under N₂ atmosphere. An internal thermocouple (J-KEM) was attached, and the mixture was placed in a preheated reaction block (225° C.) and heated to an internal temperature of 183° C. Analysis by LCMS indicated complete conversion to the desired product. The mixture was cooled to below 100° C. with vigorous stirring and diluted with hexanes. After stirring at reflux for 15 min, the mixture was cooled to rt and filtered, providing 7.1 g (48%) of U as a tan solid. The mother liquor was applied directly to a 65 g loading cartridge and purified by ISCO (220 g Column, 100% DCM to 100% EtOAc gradient) providing an additional 3.75 g (25%) of U. MS [M+H]⁺=352.23 (100%), 354.0 (90%). ¹H-NMR (400 MHz, DMSO-d6) δ 11.44 (s, 1H), 7.52-7.47 (m, 2H), 7.43-7.25 (m, 5H), 5.19 (s, 2H), 4.32 (q, J=8 Hz, 2H), 1.58 (s, 9H), 1.32 (t, J=8 Hz, 3 Hz); MS [M+H]⁺=380.17.

### Stage B

### Step I

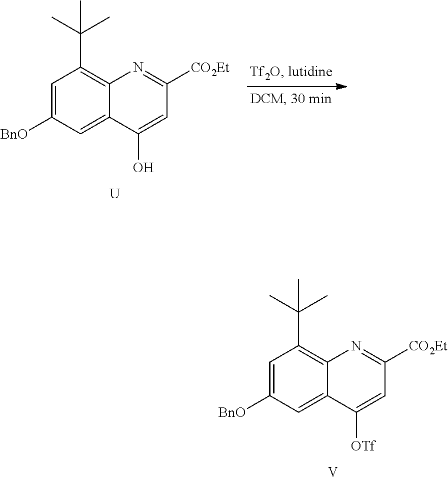

A 3-L reactor fitted with an addition funnel, a N₂ inlet and a thermocouple was charged with compound U (91 g, 240 mmol), 300 mL DCM and 2,6-lutidine (84 mL, 723 mmol). The solution was cooled to an internal temperature of less than 10° C., and a solution of trifluoromethanesulfonic anhydride (100 g, 355 mmol., single 100 g ampule) in 100 mL DCM was added over 20 min, keeping the internal temperature below 10° C. After the addition, analysis of the reaction mixture by LCMS indicated clean conversion to the desired product. The reaction was diluted with ˜1.5 L 1 N HCl and the DCM layer drained. The organic layer was washed twice with DCM, the organics were combined, dried with MgSO₄ and filtered thru a pad of silica. After removal of the solvent, the product crystallized into a very hard, solid mass. The mass was suspended in ether and carefully broken up with heating and sonication. Filtration provided the desired product (85.1 g, 70% yield) as an off-white solid. The filtrate was concentrated and the solids slurried in hexanes, then filtered to provide a second crop of the desired product (31.7 g, 25.8% yield), again as an off-white sold. Concentration of the filtrate provided a third batch of product (7.5 g, 6% yield). All 3 batches were essentially pure by 1H-NMR; LCMS rt=4.65 min; [M+H]=512.1; ¹⁹F-NMR δ −73.47 (s); ¹H-NMR (400 mHz, DMSO) δ 8.07 (s, 1H), 7.50 (m, 3H), 7.39 (m, 2H), 7.34 (m, 2H), 7.22 (d, 2H, J=2 Hz), 5.29 (s, 2H), 4.39 (q, J=7 Hz, 2H), 1.59 (s, 9H), 1.43 (t, J=7 Hz, 3H).

### Step II

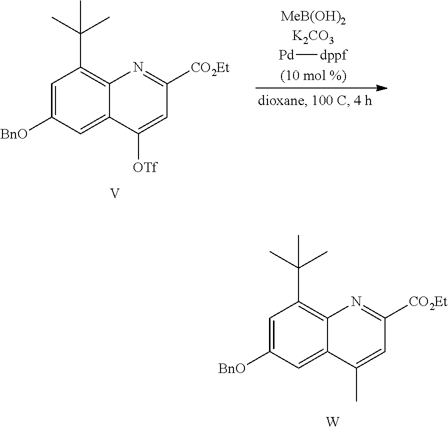

A 3-L reactor fitted with a thermocouple and a N₂ inlet was charged with 800 mL dioxane, triflate V (124 g, 240 mmol), methylboronic acid (35.2 g, 587 mmol) and K₂CO₃ (108 g, 782 mmol). The mixture was degassed by stirring under vacuum and backfilling with N₂ (3×). [1,1′-Bis(diphenylphosphino)ferrocene]palladium(II) chloride, complex with dichloromethane (1:1) (16 g, 19.6 mmol) was added and the mixture was heated to 100° C. After 1 h, analysis by LCMS indicated that the reaction was complete. The reaction mixture was cooled to rt and concentrated by rotary evaporation. The residue was suspended in 500 mL of DCM and filtered thru silica, washing well with additional DCM. The filtrate was concentrated to provide the desired product (91.4 g, 101% yield) as a light yellow solid; LCMS rt=2.81 min; [M+H]=378.1;

### Step III

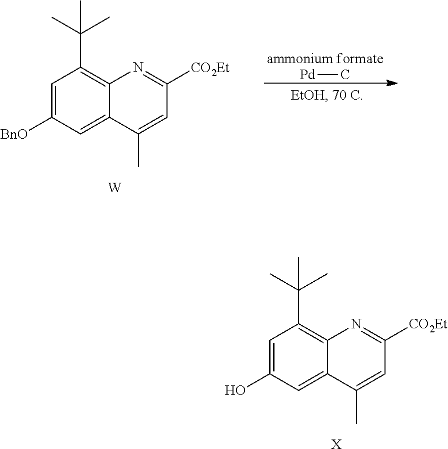

A solution of benzyl ether W (91.4 g, 242 mmol) in 1 L of EtOH was treated with ammonium formate (153 g, 2.42 mmol) and a slurry of 10% Pd—C (18.1 g, 17.2 mmol, Degussa-type E101) in ˜8 mL water. The mixture was heated to 55° C. for 1 h, then cooled to rt and filtered thru Celite (Note: filtration was very sluggish). The filtrate was concentrated and the residue partitioned between EtOAc and water. The organic layer was washed with water, brine, dried with sodium sulfate and concentrated to provide the desired product (60.1 g, 87% yield); LCMS rt=2.63 min; [M+H]=288.1; ¹H-NMR (400 mHz, DMSO) δ 10.22 (s, 1H), 7.82 (s, 1H), 7.26 (s, 1H), 7.10 (s, 1H), 4.39 (q, J=7 Hz, 2H), 2.57 (s, 3H), 1.58 (s, 9H), 1.32 (t, J=7 Hz, 3H).

### Step IV

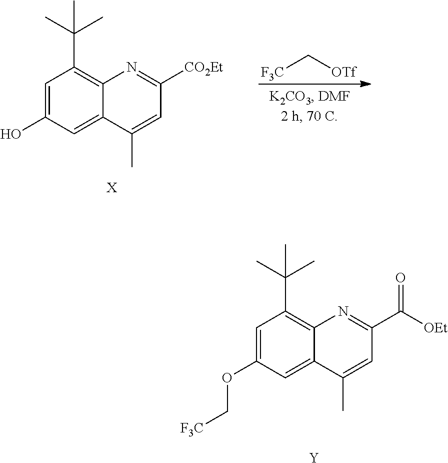

To a solution of ethyl 8-tert-butyl-6-hydroxy-4-methylquinoline-2-carboxylate X (28 g, 97.6 mmol) in 400 mL DMF was added powdered potassium carbonate (27 g, 195.6 mmol). The reaction mixture was stirred and 2,2,2-trifluoroethyl trifluoromethanesulfonate (34 g, 146.2 mmol) was added. The reaction was heated at 70° C. for 3 h and was cooled to 0° C. Water (10 was added. A light-yellow precipitate formed. It was filtered, washed with water, and dried to give the product as a light-yellow solid (35.8 g, 100%); LCMS rt=2.75 min; [M+H]=370.1; ¹H-NMR (400 mHz, DMSO) δ 7.92 (s, 1H), 7.41 (s, 1H), 7.32 (s, 1H), 4.97 (q, J=9 Hz, 2H), 4.35 (q, J=7 Hz, 2H), 2.69 ((s, 3H), 1.59 (s, 9H), 1.34 (t, J=7 Hz, 3H).

### Step V

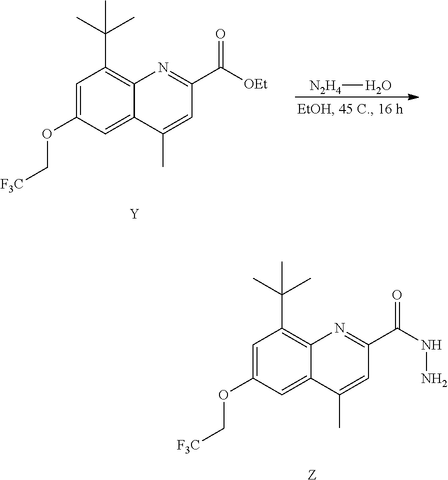

Hydrazine hydrate Y (17.8 ml, 365.7 mmol) was added to a suspension of ethyl 8-tert-butyl-4-methyl-6-(2,2,2-trifluoroethoxy)quinoline-2-carboxylate (27 g, 73.1 mmol) in 300 mL EtOH. The reaction mixture was stirred at 70° C. for 2 h and then was concentrated to remove EtOH. Water (500 mL) was added. A light-yellow precipitate formed. It was filtered, washed with water, and dried to give the product Z as a light-yellow solid (26 g, 100%); LCMS rt=2.42 min; [M+H]=356.1; ¹⁹F-NMR δ −72.8 (t); ¹H-NMR (400 mHz, DMSO) δ 8.87 (s, 1H), 7.93 (s, 1H), 7.42 (d, J=3 Hz, 1H), 7.33 (d, J=3 Hz, 1H), 4.96 (d, J=9 Hz. 2H), 4.69 (s, 2H), 2.70 (s, 3H), 1.60 (s, 9H).

### Compound 448

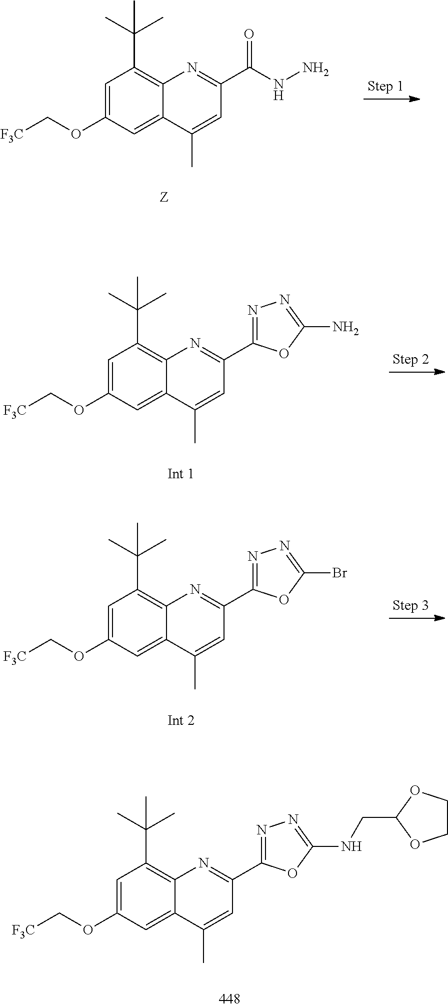

### Step 1:

Int 1 was made from Z according to procedures described in Step 3 of Example 31.

### Step 2:

Int 2 was made according to procedures described in Step 4 of Example 31.

### Step 3:

The procedures described in Step 5 of Example 31 were followed to give compound 448 as a white solid.

¹H NMR (400 MHz, DMSO-d6) δ 8.19 (d, J=6.3 Hz, 1H), 7.91 (s, 1H), 7.40 (s, 1H), 7.32 (s, 1H), 5.04 (t, J=4.3 Hz, 1H), 4.96 (q, J=8.9 Hz, 2H), 3.92 (t, J=6.8 Hz, 2H), 3.80 (t, J=6.9 Hz, 2H), 3.41-3.35 (m, 2H), 2.69 (s, 3H), 1.61 (s, 9H); ¹⁹F NMR (376.1 MHz) δ −72.82, −75.17 (TFA salt); MS [M+H]⁺=467.2; LC/MS RT=2.58 min.

### Compounds 449-456

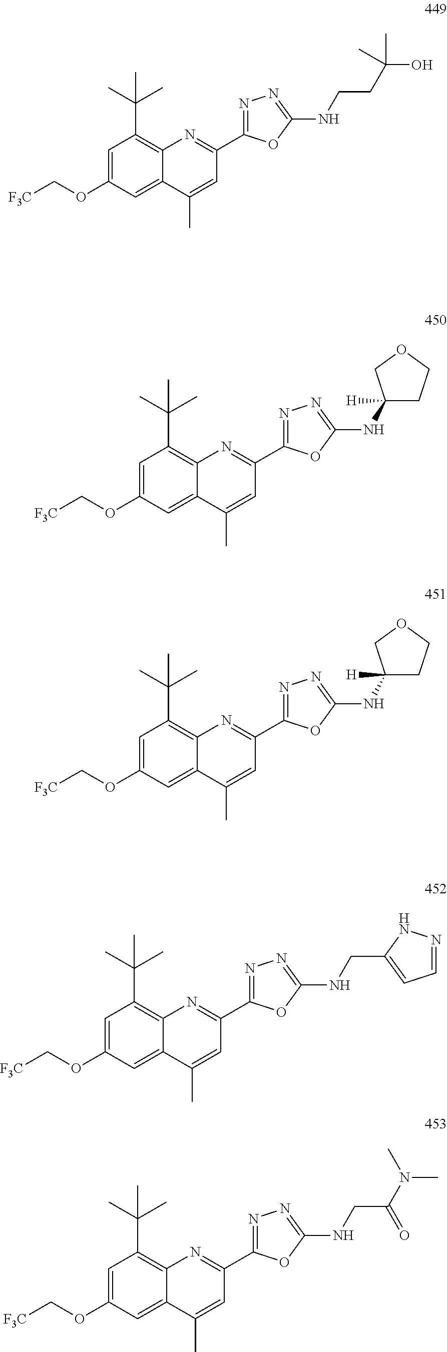

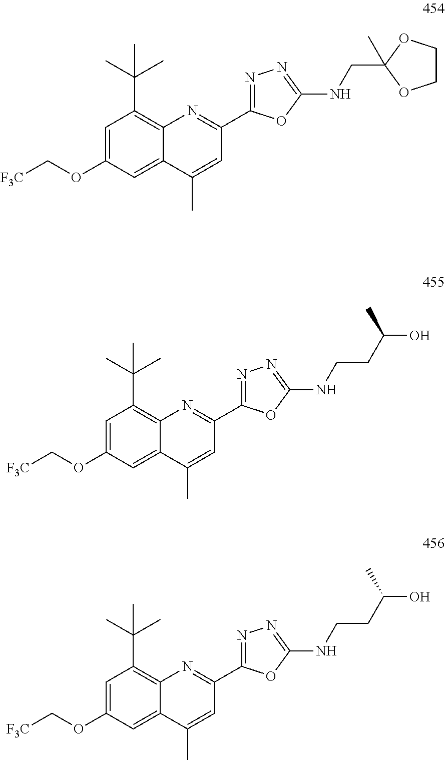

The compounds in the example were made according to procedures described previously.

449: ¹H NMR (400 MHz, DMSO-d6) δ 7.91 (s, 1H), 7.87 (s, 1H), 7.40 (s, 1H), 7.31 (s, 1H), 5.01-4.90 (m, 2H), 3.34 (s, 2H), 2.69 (s, 3H), 1.76-1.65 (m, 2H), 1.61 (s, 9H), 1.12 (s, 6H); ¹⁹F NMR (376.1 MHz) δ −72.82, −75.15 (TFA salt); MS [M+H]⁺=467.2; LC/MS RT=2.38 min.

450: ¹H NMR (400 MHz, DMSO-d6) δ 8.24 (d, J=5.8 Hz, 1H), 7.92 (s, 1H), 7.41 (s, 1H), 7.32 (d, J=2.6 Hz, 1H), 4.96 (q, J=8.7 Hz, 2H), 4.20 (s, 1H), 3.83 (dd, J=15.7, 8.3 Hz, 2H), 3.76-3.64 (m, 2H), 2.69 (s, 3H), 2.18 (dd, J=13.0, 7.8 Hz, 1H), 1.96 (s, 1H), 1.62 (s, 9H); ¹⁹F NMR (376.1 MHz) δ −72.82, −75.24 (TFA salt); MS [M+H]⁺=451.3; LC/MS RT=2.45 min.

451: ¹H NMR (400 MHz, DMSO-d6) δ 8.24 (d, J=5.6 Hz, 1H), 7.92 (s, 1H), 7.41 (s, 1H), 7.32 (d, J=2.8 Hz, 1H), 4.95 (t, J=8.8 Hz, 2H), 4.20 (s, 1H), 3.83 (dd, J=15.8, 8.3 Hz, 2H), 3.70 (dd, J=10.5, 7.1 Hz, 2H), 2.69 (s, 3H), 2.17 (d, J=8.0 Hz, 1H), 1.96 (s, 1H), 1.62 (s, 9H); ¹⁹F NMR (376.1 MHz) δ −72.82, −75.20 (TFA salt); MS [M+H]⁺=451.3; LC/MS RT=2.47 min.

452: ¹H NMR (400 MHz, DMSO-d6) δ 8.41 (s, 1H), 7.92 (s, 1H), 7.40 (s, 1H), 7.32 (s, 1H), 5.01-4.91 (m, 2H), 4.44 (s, 2H), 2.69 (s, 3H), 1.61 (s, 9H); 19F NMR (376.1 MHz) δ −72.82, −75.08 (TFA salt); MS [M+H]⁺=461.3; LC/MS RT=2.34 min.

453: ¹H NMR (400 MHz, DMSO-d6) δ 8.11 (s, 1H), 7.92 (s, 1H), 7.41 (d, J=2.6 Hz, 1H), 7.32 (d, J=2.7 Hz, 1H), 4.96 (q, J=8.7 Hz, 3H), 4.11 (d, J=5.9 Hz, 2H), 2.99 (s, 3H), 2.82 (s, 3H), 2.69 (s, 3H), 1.61 (s, 9H); ¹⁹F NMR (376.1 MHz) δ −72.82, −75.29 (TFA salt); MS [M+H]⁺=466.2; LC/MS RT=2.38 min.

454: ¹H NMR (400 MHz, DMSO-d6) δ 8.13 (s, 1H), 7.91 (s, 1H), 7.40 (s, 1H), 7.32 (s, 1H), 4.96 (q, J=9.0 Hz, 2H), 3.90 (d, J=8.4 Hz, 4H), 3.36 (d, J=6.5 Hz, 2H), 2.69 (s, 3H), 1.62 (s, 9H), 1.30 (s, 3H); ¹⁹F NMR (376.1 MHz) δ −72.82, −75.13 (TFA salt); MS [M+H]⁺=481.3; LC/MS RT=2.58 min.

455: ¹H NMR (400 MHz, DMSO-d6) δ 7.91 (s, 2H), 7.40 (s, 1H), 7.32 (s, 1H), 4.95 (d, J=9.0 Hz, 2H), 3.69 (s, 2H), 3.31 (s, 2H), 2.69 (s, 3H), 1.61 (s, 12H), 1.07 (d, J=6.1 Hz, 3H); ¹⁹F NMR (376.1 MHz) δ −72.83, −74.79 (TFA salt); MS [M+H]⁺=453.2; LC/MS RT=2.44 min.

456: ¹H NMR (400 MHz, DMSO-d6) δ 7.90 (d, J=5.1 Hz, 2H), 7.40 (s, 1H), 7.32 (s, 1H), 4.94 (t, J=8.9 Hz, 2H), 3.74-3.65 (m, 1H), 2.69 (s, 3H), 1.61 (s, 11H), 1.07 (d, J=6.2 Hz, 3H); ¹⁹F NMR (376.1 MHz) δ −72.83, −73.98 (TFA salt); MS [M+H]⁺=453.2; LC/MS RT=2.43 min.

### Compound 457

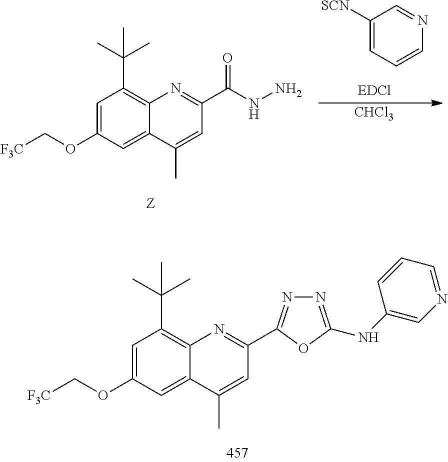

The compound in the example was made according to procedures described previously.

¹H NMR (400 MHz, DMSO-d6) δ 11.21 (s, 1H), 8.89 (s, 1H), 8.32 (d, J=4.7 Hz, 1H), 8.23 (s, 1H), 8.02 (s, 1H), 7.56 (s, 1H), 7.44 (s, 1H), 7.36 (s, 1H), 4.98 (q, J=9.0 Hz, 2H), 2.73 (s, 3H), 1.66 (s, 9H); ¹⁹F NMR (376.1 MHz) δ −72.80, −74.96 (TFA salt); MS [M+H]⁺=458.3; LC/MS RT=2.53 min.

### Compound 458

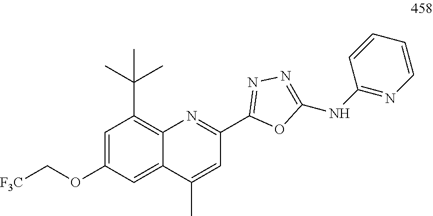

The compound in the example was made according to procedures described previously.

¹H NMR (400 MHz, DMSO-d6) δ 8.29-8.24 (m, 1H), 8.00 (s, 1H), 7.85 (s, 2H), 7.43 (s, 1H), 7.35 (s, 1H), 7.05-6.99 (m, 1H), 4.97 (d, J=8.6 Hz, 2H), 2.72 (s, 3H), 1.65 (s, 9H); ¹⁹F NMR (376.1 MHz) δ −72.78, −74.89 (TFA salt); MS [M+H]⁺=458.2; LC/MS RT=2.59 min.

### Compound 459

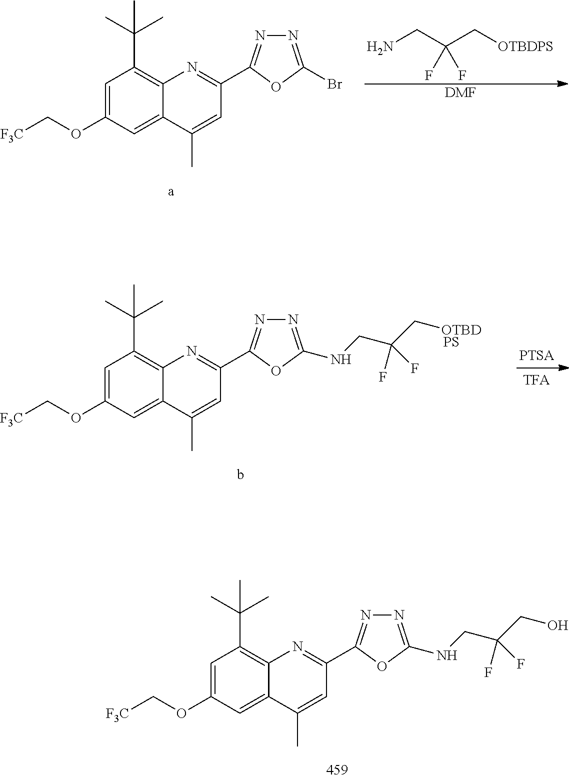

### Step 1:

The procedures described in Step 5 of Example 31 were followed to give b.

### Step 2:

PTSA (300 mg) was added to b (160 mg, 0.225 mmol) dissolved in TFA (1.5 mL). The reaction mixture was stirred at it for 2 d. It was then diluted with water and extracted with EtOAc. The organic layer was concentrated and purified on prep HPLC to give compound 459 as an off-white solid (15 mg, 13%).

¹H NMR (400 MHz, DMSO-d6) δ 8.44 (t, J=6.4 Hz, 1H), 7.92 (s, 1H), 7.41 (s, 1H), 7.32 (d, J=2.7 Hz, 1H), 5.56 (t, J=6.3 Hz, 1H), 4.96 (q, J=8.8 Hz, 2H), 3.77 (td, J=14.4, 6.1 Hz, 2H), 3.67 (td, J=13.4, 6.3 Hz, 2H), 2.69 (s, 3H), 1.62 (s, 10H), 0.90 (dd, J=15.8, 9.7 Hz, 3H); ¹⁹F NMR (376.1 MHz) δ −72.83, −73.94, −112.78 (TFA salt); MS [M+H]⁺=475.3; LC/MS RT=2.37 min.

### Compounds 460

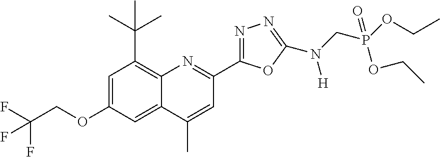

This compound was prepared analogously to 448 employing an appropriate amine.

¹H NMR (400 MHz, dmso) δ 8.49 (d, J=6.3 Hz, 1H), 7.93 (s, 1H), 7.41 (d, J=2.6 Hz, 1H), 7.32 (d, J=2.7 Hz, 1H), 4.96 (q, J=8.9 Hz, 2H), 4.10-3.96 (m, 4H), 3.74 (dd, J=9.9, 6.4 Hz, 2H), 2.69 (s, 3H), 1.62 (s, 9H), 1.18 (t, J=7.0 Hz, 6H); ¹⁹F NMR (376 MHz, dmso) δ −72.80, −72.82, −72.85, −74.79; ³¹P NMR (162 MHz, dmso) δ 22.26; MS [M+H]⁺=531.17.

### Compounds 461-463

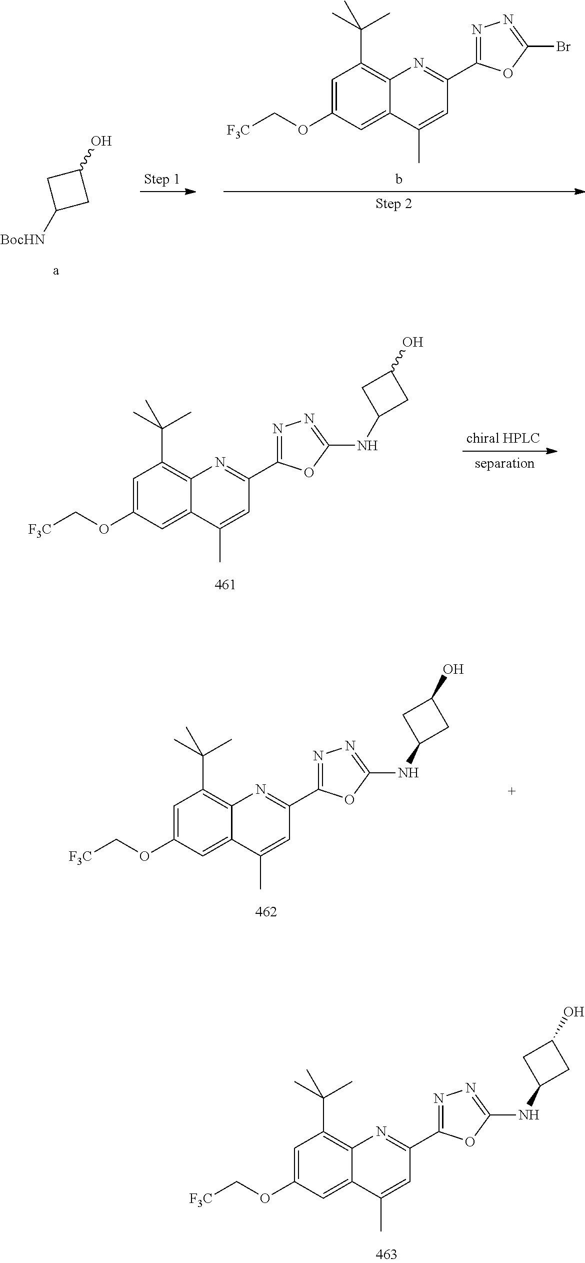

Compound a (356 mg, 1.90 mmol) was treated with HCl in dioxane to remove Boc protecting group as described previously.

Compound 460 (91 mg, 44%, ˜7:3 mixture of cis and trans isomers) was prepared from compound b (202 mg, 0.46 mmol) in a manner similar to that described previously.

### Compound 461

¹H-NMR (400 MHz, CDCl₃) δ 7.94 (s, 1H), 7.45 (s, 1H), 7.10 (d, J=1.6 Hz, 1H), 4.80 (br, 2.6H), 4.51 (q, J=7.8 Hz, 2H), 4.20 (br, 0.7H), 3.88 (br, 0.7H), 3.01 (br, 1.4H), 2.71 (s, 3H), 2.57 (br, 1.2H), 2.23 (br, 1.4H), 1.68 (s, 9H); ¹⁹F NMR (376.1 MHz, CDCl₃) δ −74.10 (t, J=7.7 Hz, 3F), −76.53 (s, 3F); MS [M+H]⁺=451.3

Two isomers were separated by preparative chiral HPLC.

Compound 462: (38.7 mg): ¹H-NMR (400 MHz, CD₃OD) δ 7.89 (s, 1H), 7.40 (d, J=2.8 Hz, 1H), 7.29 (d, J=2.8 Hz, 1H), 4.71 (q, J=8.4 Hz, 2H), 4.03 (q, J=7.3 Hz, 1H), 3.71 (m, 1H), 2.84 (m, 2H), 2.70 (s, 3H), 2.00 (m, 2H), 1.66 (s, 9H); ¹⁹F NMR (376.1 MHz, CDCl₃) δ −75.91 (t, J=8.3 Hz, 3F); MS [M+H]⁺=451.3

Compound 463: (14.3 mg): ¹H-NMR (400 MHz, CDCl₃) δ 7.92 (s, 1H), 7.40 (d, J=2.8 Hz, 1H), 7.31 (d, J=2.8 Hz, 1H), 4.72 (q, J=8.4 Hz, 2H), 4.49 (quintet, J=6.1 Hz, 1H), 4.26 (m, 1H), 2.712 (s, 3H), 2.42 (m, 4H), 1.68 (s, 9H); ¹⁹F NMR (376.1 MHz, CDCl₃) δ −75.80 (t, J=8.4 Hz, 3F); MS [M+H]⁺=451.3

### Compounds 464-465

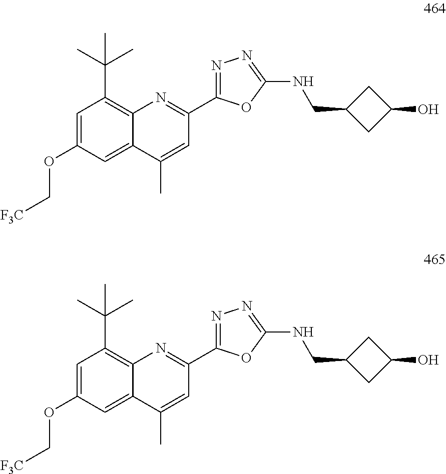

Compound 464: ¹H-NMR (400 MHz, DMSO-d₆) δ 8.02 (m, 1H), 7.91 (s, 1H), 7.40 (m, 1H), 7.31 (m, 1H), 4.95 (q, 2H), 3.88 (m, 1H), 3.25 (m, 4H), 2.69 (s, 3H), 2.25 (m, 2H), 2.00 (m, 1H), 1.61 (s, 9H), 1.52 (m, 2H). ¹⁹F NMR (376.1 MHz) 6-72.83 (t), −75.17 (s); MS [M+H]⁺=465.3

Compound 465: ¹H-NMR (400 MHz, DMSO-d₆) δ 8.04 (m, 1H), 7.91 (s, 1H), 7.40 (m, 1H), 7.31 (m, 1H), 4.95 (q, 2H), 4.21 (m, 1H), 3.28 (m, 4H), 2.69 (s, 3H), 2.38 (m, 1H), 2.00 (m, 2H), 1.92 (m, 2H), 1.61 (s, 9H). ¹⁹F NMR (376.1 MHz) 6-72.82 (t), −75.17 (s); MS [M+H]⁺=465.3

### Compound 466

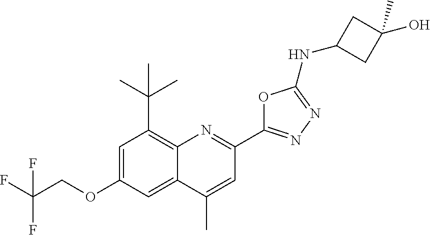

¹H-NMR (400 MHz, MeOD) δ 7.91 (s, 1H), 7.39 (d, 1H), 7.3 (d, 1H), 4.7 (q, 2H), 3.79 (m, 1H), 2.71 (s, 3H), 2.54 (m, 2H), 2.15 (m, 2H), 1.66 (s, 9H), 1.37 (s, 3H)

¹⁹F NMR (376.1 MHz) δ −75.99 (t)

MS [M+H]⁺=465.2

### Compound 467

### Step I

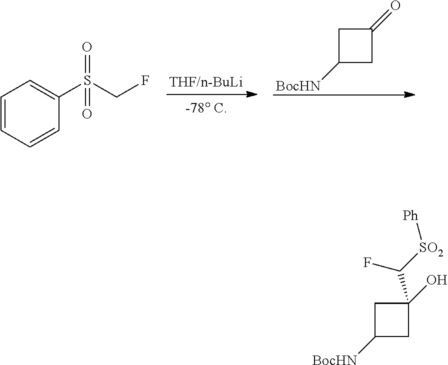

Fluoromethylphenylsulfone (17.4 g, 100 mmol) was dissolved in THF (150 ml), followed by the addition of 2.5 N of n-BuLi in Hexane solution (40 ml, 100 mmol) at −78° C., After 30 min. cyclobutanone (9.25 g, 50 mmol) in 50 ml THF solution was added. The reaction was stirred at −78° C. for 2 h. After warm to room temperature, the reaction was quenched with saturated NH₄Cl water solution and extracted with ethyl acetate. The extract was dried and purified silica gel column, the purified material was crystallized from the mixture of ethyl acetate and hexane to afford 8.82 g, 98% pure cis product.

¹H-NMR (400 MHz, CDCl3) δ 7.93 (m, 2H), 7.72 (m, 1H), 7.6 (m, 2H), 5 (d, 1H), 4.87 (br., 1H), 3.88 (m, 2H), 3.0 (m, 2H), 2.1 (m, 2H), 1.42 (s, 9H)

MS [M+H]⁺=359.51

### Step II

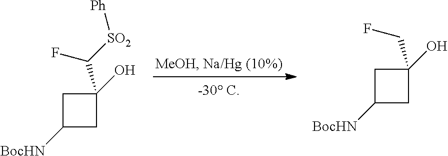

Phenylsulfone (8.8 g, 24.5 mmol) was dissolved in MeOH (100 ml), followed by the addition of Na₂HPO₄ (20.88 g, 147 mmol) and Na/Hg (10%, 28.2 g, 122.5 mmol) at −30° C., After 30 min. the reaction was filtered and the MeOH was removed, the product was crystallized from the mixture of ethyl acetate and hexane to afford 4.47 g of pure cis product.

¹H-NMR (400 MHz, CDCl3) δ 4.75 (br., 1H), 4.3 (d, 2H), 3.7 (br., 1H), 2.62 (m, 2H), 2.0 (m, 2H), 1.42 (s, 9H)

### Step III

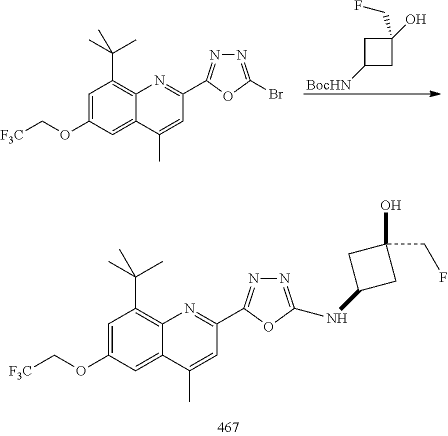

Compound 467 was prepared in the manner similar to compound 461.

¹H-NMR (400 MHz, CD₃OD) δ 7.91 (s, 1H), 7.41 (m, 1H), 7.11 (m, 1H), 4.72 (m, 2H), 4.42, 4.36 (d, 2H), 3.85 (m, 1H), 2.78 (m, 2H), 2.72 (s, 3H), 2.20 (m, 2H), 1.62 (s, 9H). ¹⁹F NMR (376.1 MHz) δ −72.02, −73.98 (d), −225.06 (t); MS [M+H]⁺=483.4

Alternatively, 467 was prepared from the above intermediate and intermediate Z in this example in the manner similar to example compound 457 via intermediates prepared according to the procedures:

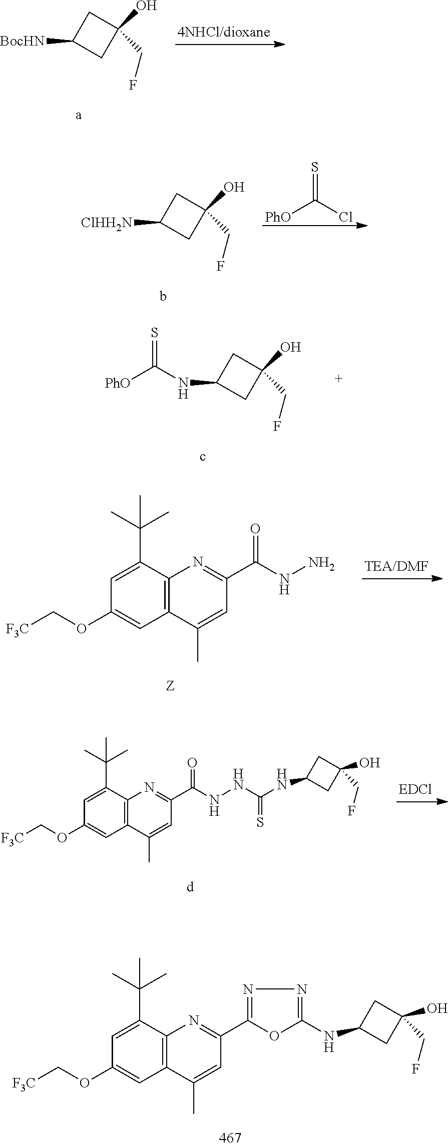

A 100-mL 1-neck rbf was charged with intermediate a (1.36 g, 6.2 mmol) and DCM (5 mL). A solution of 4 N HCl in dioxane (8 mL) was added dropwise to the reaction mixture with stirring at room temperature. The reaction mixture was stirred for 1 hour until TLC indicated intermediate 1 was gone. After removal of the solvent in vacuo, intermediate b (˜1.0 g) was obtained and used for next step without further purification.

A 100-mL 1-neck rbf was charged with intermediate b (1.0 g, 6.2 mmol), TEA (1.44 g, 14.3 mmol) and DCM (10 mL). The reaction mixture was cooled to 0° C. Phenyl chlorothionformate (1.1 g, 6.2 mmol) was added drop wise to the reaction mixture with stirring. The reaction mixture was warmed to room temperature and maintained stirring for another 2 hours. After removal of the solvent in vacuo, the residue was dissolved in EtOAc (50 mL) and washed by H₂O (30 mL) and brine (30 mL) and dried by Na₂SO₄. After concentration, the residue was purified by flash chromatography (silica gel, ethyl acetate/hexane gradient) affording 1.55 g (˜90% pure) of intermediate c as yellow oil.

A 100-mL 1-neck rbf was charged with intermediate c (1.4 g from 1.55 g 90% pure crude, 5.5 mmol), TEA (0.56 g, 5.5 mmol), intermediate Z (1.95 g, 5.5 mmol) and DMF (20 mL). The reaction mixture was heated to 65° C. for 0.5 hour, LC-MS indicated all intermediate Z converted to intermediate d. The reaction was cooled back to room temperature. EDCl (1.6 g, 8.2 mmol) was added in. The reaction mixture was heated to 65° C. for another 0.5 hour with stirring. LC-MS indicated all intermediate d converted to compound 467. The reaction mixture was cooled to room temperature and diluted with EtOAc (3000 mL) and washed by H₂O (100 mL), 5% LiCl (100 mL) and dried by Na₂SO₄. After concentration, the residue was purified by flash chromatography (silica gel, ethyl acetate/hexane gradient) affording 2.3 g compound 467 as a white solid.

### Compound 468

### Step I

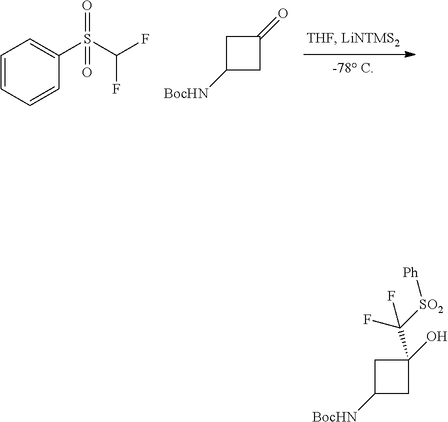

Difluoromethylphenylsulfone (5 g, 27 mmol) and cyclobutanone (5.19 g, 27 mmol) were dissolved in THF (100 ml), followed by the addition of 1 N of LiNTMS₂ in THF solution (54 ml, 54 mmol) at −78° C. The reaction was stirred at −78° C. for 2 h. After warm to room temperature, the reaction was quenched with saturated NH₄Cl water solution and extracted with ethyl acetate. The extract was dried and purified and recrystallized from the mixture of ethyl acetate and hexane to afford 5.25 g pure cis product.

¹H-NMR (400 MHz, CDCl3) δ 7.97 (m, 2H), 7.75 (m, 1H), 7.6 (m, 2H), 4.9 (br., 1H), 4.1 Br., 1H), 3.87 (m, 1H), 3.18 (m, 2H), 2.28 (m, 2H), 1.42 (s, 9H)

¹⁹F NMR (376.1 MHz) δ −112.45 (s)

### Step II

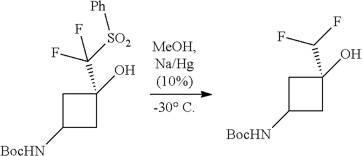

Procedure is same as for example compound 467.

¹H-NMR (400 MHz, CDCl3) δ 5.65 (t, 1H), 4.85 (br., 1H), 3.75 (m, 1H), 3.0 (br., 1H), 2.82 (m, 2H), 2.1 (m, 2H), 1.42 (s, 9H)

¹⁹F NMR (376.1 MHz) δ −133.9 (d)

### Step III: Compound 468

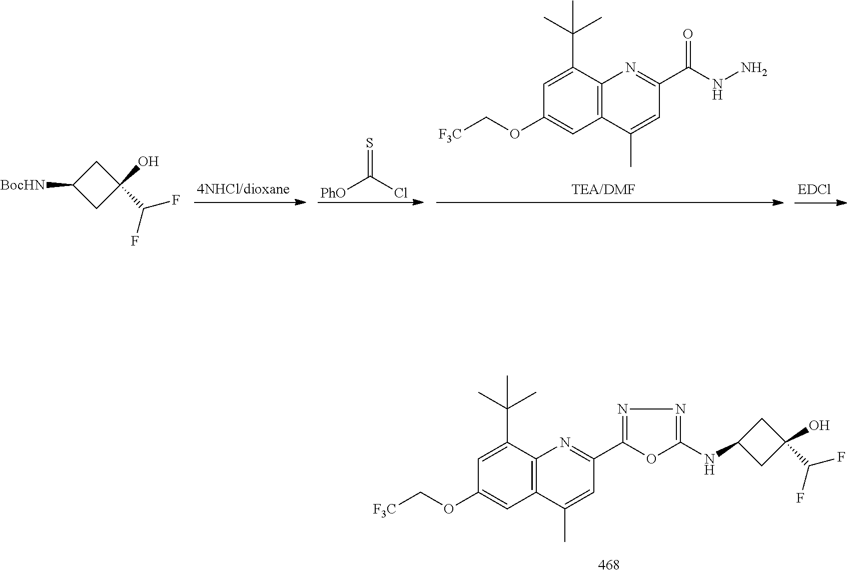

Compound 468 was prepared in the manner similar to example compound 467.

¹H-NMR (400 MHz, MeOD-d) δ 7.91 (s, 1H), 7.4 (d, 1H), 7.3 (d, 1H), 5.768 (t, 1H), 4.72 (q, 2H), 3.92 (m, 1H), 2.92 (m, 2H), 2.71 9s, 3H), 2.2 (m, 2H), 1.66 (s, 9H)

¹⁹F NMR (376.1 MHz) δ −75.99 (t), −135.27 (d)

MS [M+H]⁺=501.18

### Compound 469

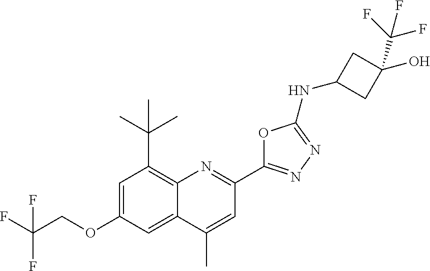

Compound 469 was prepared in the manner similar to compound 467.

¹H-NMR (400 MHz, CDCl3) δ 7.98 (s, 1H), 7.42 (d, 1H), 7.07 (d, 1H), 6.22 (s, 1H), 4.49 (q, 2H), 4.1 (s, 1H), 3.13 (m, 2H), 2.68 (s, 3H), 2.66 (m, 2H), 1.65 (s, 9H)

¹⁹F NMR (376.1 MHz) δ −74.13 (t), −84.89 (s)

MS [M+H]⁺=519.24

### Compounds 470

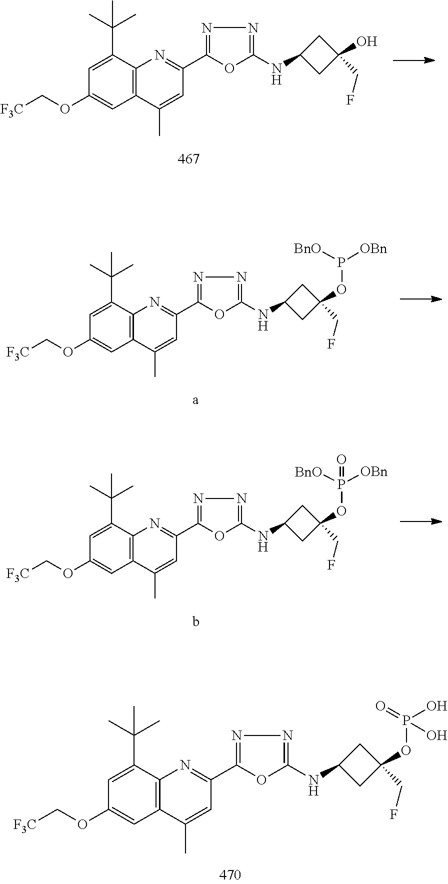

467 (44 mg, 0.091 mmol) dissolved in DCE (5 ml) was added dibenzyl-N,N-diisopropylphosphanate (94.76 mg, 0.27 mmol) and 1,2,4-triazole (18.6 mg, 0.27 mmol). After reflux for 4 h (Attached LC-MS), added dibenzyl-N,N-diisopropylphosphanate (49 mg) and 1,2,4-triazole (10 mg) and heated to reflux again for 4 h (attached LC-MS).

After cooled to room temperature, Hydrogen peroxide (30%, 2 ml) was added and stirred for 0.5 h, LC-MS show the completion of the reaction. The reaction mixture was diluted with EtOAc (100 ml) and washed with 10% of Na2S2O3 solution and brine. The organic layer was dried (Na₂SO₄) and concentrated. The residue was purified by flash chromatography on silica gel with EA/Hex to give 40 mg of phosphate.

Dibenzyl phosphate b (40 mg) dissolved in EtOH (10 ml) was added 10% Pd/C (25 mg), then under hydrogen (balloon pressure) for 1 h. The catalyst was remover through celite filtration. After removed the solvent, and crystallization from DCM/Hexane, yielded 22.5 mg of compound 470.

¹H-NMR (400 MHz, d-DMSO) δ 8.5 (d, 1H), 7.9 (s, 1H), 7.4 (d, 1H), 7.31 (d, 1H), 4.95 (q, 2H), 4.63 (s, 1H), 4.51 (s, 1H), 3.8 (m, 1H), 2.68 (s, 3H), 2.61 (m, 2H), 2.4 (m, 2H), 1.61 (s, 9H)

¹⁹F NMR (376.1 MHz) δ −72.83 (t), −226.15 (t)

MS [M+H]⁺=563.17

### Compounds 471-473

Compounds 471 to 473 were prepared in the manner similar to compound 470.

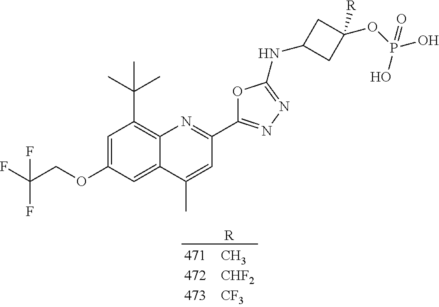

### Compound 471

¹H-NMR (400 MHz, d-DMSO) δ 8.32 (s, 1H), 7.93 (s, 1H), 7.4 (d, 1H), 7.32 (d, 1H), 4.72 (q, 2H), 4.49 (m, 1H), 3.81 (m, 1H), 2.94 (m, 2H), 2.72 (s, 3H), 2.25 (m, 2H), 1.67 (s, 9H)

¹⁹F NMR (376.1 MHz) δ −76.01 (t)

MS [M+H]⁺=531.17

### Compound 472

¹H-NMR (400 MHz, DMSO-d₆) δ 8.55 (d, 1H), 7.92 (s, 1H), 7.41 (m, 1H), 7.31 (m, 1H), 6.25 (t, 1H), 4.96 (m, 2H), 3.81 (m, 1H), 2.81 (m, 2H), 2.76 (m, 2H), 2.72 (s, 3H), 1.61 (s, 9H). ¹⁹F NMR (376.1 MHz) δ −72.81 (t), −133.72 (d); ³¹P NMR (400 MHz) δ −4.30 (s); MS [M+H]⁺=581.13

### Compound 473

¹H-NMR (400 MHz, d-DMSO) δ 7.89 (s, 1H), 7.39 (s, 1H), 7.3 (s, 1H), 5.46 (s, 2H), 4.71 (q, 2H), 4 (s, 1H), 2.99 (s, 1H), 2.7 (s, 3H), 1.65 (s, 9H)

¹⁹F NMR (376.1 MHz) δ −75.97 (t), −85.92 (s)

MS [M+H]⁺=599.19

### Compound 474 (racemate)

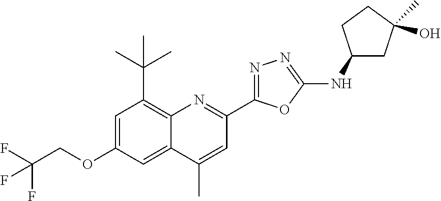

This compound was prepared analogously to 448 employing an appropriate amine. This compound is racemic. It was prepared from the less polar isomer of N-Boc 1-methyl-3-aminocyclopentanol resulting from the action of methyl lithium on N-Boc cyclopentan-3-one, as per example 222/223 above.

¹H NMR (400 MHz, dmso) δ 7.97 (d, J=6.6 Hz, 1H), 7.91 (s, 1H), 7.41 (d, J=2.7 Hz, 1H), 7.31 (d, J=2.7 Hz, 1H), 4.95 (q, J=8.9 Hz, 2H), 4.50 (s, 1H), 3.95 (d, J=7.8 Hz, 1H), 2.69 (s, 3H), 2.02 (dd, J=13.1, 8.4 Hz, 2H), 1.88-1.65 (m, 3H), 1.61 (s, 9H), 1.54-1.42 (m, 1H), 1.21 (s, 3H); ¹⁹F NMR (376 MHz, dmso) δ −72.80, −72.83, −72.85; MS [M+H]⁺=479.13.

### Compound 475 (racemate)

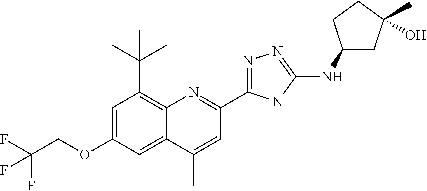

This compound was prepared analogously to 448 employing an appropriate amine. This compound is racemic. It was prepared from the more polar isomer of N-Boc 1-methyl-3-aminocyclopentanol resulting from the action of methyl lithium on N-Boc cyclopentan-3-one, as per example 222/223 above.

¹H NMR (400 MHz, dmso) δ 7.97 (d, J=6.6 Hz, 1H), 7.91 (s, 1H), 7.41 (d, J=2.7 Hz, 1H), 7.31 (d, J=2.7 Hz, 1H), 4.95 (q, J=8.9 Hz, 2H), 4.50 (s, 1H), 3.95 (d, J=7.8 Hz, 1H), 2.69 (s, 3H), 2.02 (dd, J=13.1, 8.4 Hz, 2H), 1.88-1.65 (m, 3H), 1.61 (s, 9H), 1.54-1.42 (m, 1H), 1.21 (s, 3H); ¹⁹F NMR (376 MHz, dmso) δ −72.80, −72.83, −72.85; [M+H]⁺=479.23

### Compound 476 (racemate)

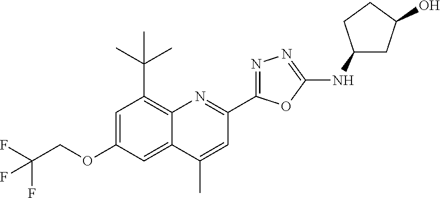

Prepared employing 448 and an appropriate racemic amine.

¹H NMR (400 MHz, dmso) δ 8.00 (d, J=6.6 Hz, 1H), 7.91 (s, 1H), 7.40 (s, 1H), 7.31 (d, J=2.7 Hz, 1H), 4.95 (q, J=8.8 Hz, 2H), 4.08 (d, J=5.0 Hz, 1H), 3.88 (d, J=6.9 Hz, 1H), 2.69 (s, 3H), 2.26 (dd, J=13.0, 6.7 Hz, 2H), 1.94 (s, 1H), 1.72 (s, 2H), 1.62 (s, 9H), 1.51 (s, 2H); ¹⁹F NMR (376 MHz, dmso) δ −72.80, −72.83, −72.85, −74.45; MS [M+H]⁺=465.25.

### Compound 477 (racemate)

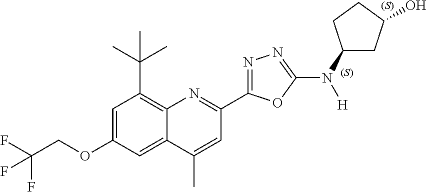

¹H NMR (400 MHz, dmso) δ 7.95 (d, J=6.8 Hz, 1H), 7.91 (s, 1H), 7.40 (d, J=2.6 Hz, 1H), 7.31 (d, J=2.7 Hz, 1H), 4.95 (q, J=8.9 Hz, 2H), 4.52 (d, J=3.8 Hz, 1H), 4.21 (d, J=3.2 Hz, 1H), 4.12 (dd, J=13.7, 6.9 Hz, 1H), 2.69 (s, 3H), 2.12 (dd, J=12.2, 7.2 Hz, 1H), 1.90 (dt, J=14.7, 7.2 Hz, 2H), 1.82-1.67 (m, 1H), 1.61 (s, 9H), 1.50 (s, 2H); ¹⁹F NMR (376 MHz, dmso) δ −72.80, −72.83, −72.85; [M+H]⁺=465.23.

### Compound 478

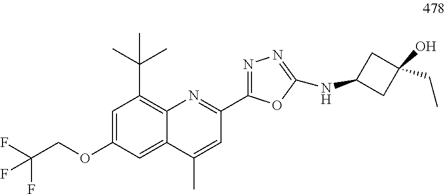

¹H-NMR (400 MHz, CH₃OH-d₄) δ 7.86 (s, 1H), 7.40 (s, 1H), 7.29 (s, 1H), 4.76 (m, 2H), 3.78 (m, 1H), 2.69 (s, 3H), 2.62 (m, 2H), 2.18 (m, 2H), 1.65 (m, 11H), 0.97 (m, 3H); ¹⁹F NMR (400 MHz, CH₃OH-d₄) δ −75.21 (s); MS [M+H]⁺=479

### Compound 479

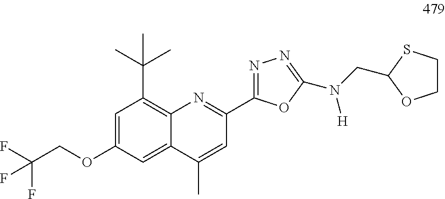

¹H NMR (400 MHz, dmso) δ 8.36 (t, J=6.0 Hz, 1H), 7.92 (s, 1H), 7.41 (d, J=2.6 Hz, 1H), 7.32 (d, J=2.7 Hz, 1H), 5.32 (dd, J=7.1, 4.4 Hz, 1H), 4.96 (q, J=8.8 Hz, 2H), 4.22-4.15 (m, 1H), 3.88 (dt, J=9.3, 6.5 Hz, 1H), 3.65 (dd, J=13.5, 5.5 Hz, 1H), 3.60-3.51 (m, 1H), 3.51-3.39 (m, 2H), 3.00 (t, J=6.4 Hz, 2H), 2.69 (s, 3H), 1.62 (s, 9H); ¹⁹F NMR (376 MHz, dmso) δ −72.80, −72.82, −72.85;

MS [M+H]⁺=483.16.

### Compound 480

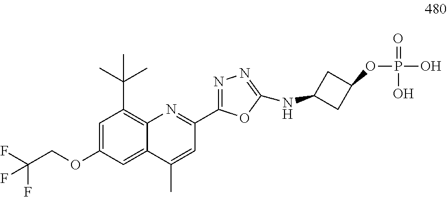

Prepared analogously to example compound 470 from compound 462.

¹H-NMR (400 MHz, CH₃OH-d₄) δ 7.84 (s, 1H), 7.40 (s, 1H), 7.29 (s, 1H), 4.76 (m, 2H), 4.46 (m, 1H), 3.80 (m, 1H), 2.95 (m, 2H), 2.69 (s, 3H), 2.30 (m, 2H), 1.69 (s, 9H); ¹⁹F NMR (400 MHz, CH₃OH-d₄) δ −75.88 (s); ³¹P NMR (400 MHz, CH₃OH-d₄) δ −1.38 (s)MS [M+H]⁺=531.

### Compound 481

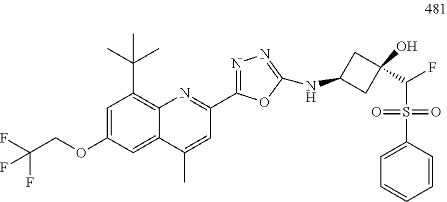

¹H-NMR (400 MHz, CH₃OH-d₄) δ 8.00 (m, 2H), 7.90 (s, 1H), 7.73 (m, 1H), 7.62 (m, 2H), 7.40 (s, 1H), 7.30 (m, 1H), 5.55 (m, 1H), 4.70 (m, 2H), 3.98 (m, 1H), 3.29 (m, 1H), 3.15 (m, 1H), 2.69 (s, 3H), 2.40 (m, 1H), 2.25 (m, 1H), 1.67 (s, 9H); ¹⁹F NMR (400 MHz, CH₃OH-d₄) δ −76.22 (t, 3F), −187.83 (d, 1F); MS [M+H]⁺=623.

### Compound 482

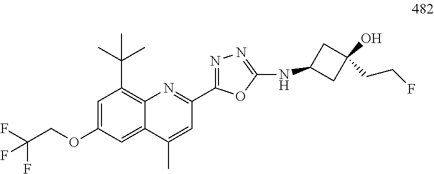

¹H-NMR (400 MHz, CH₃OH-d₄) δ 7.83 (s, 1H), 7.39 (s, 1H), 7.27 (s, 1H), 4.76 (m, 3H), 4.59 (m, 1H), 3.80 (m, 1H), 2.75 (m, 2H), 2.66 (s, 3H), 2.20 (m, 2H), 2.08 (m, 2H), 1.62 (s, 9H); ¹⁹F NMR (400 MHz, CH₃OH-d₄) δ −75.21 (s); MS [M+H]⁺=497.

### Compound 483

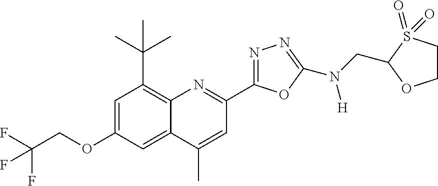

¹H NMR (400 MHz, dmso) δ 8.49 (t, J=5.8 Hz, 7H), 7.94 (s, 7H), 7.41 (d, J=2.6 Hz, 8H), 7.32 (d, J=2.7 Hz, 8H), 4.96 (q, J=9.0 Hz, 20H), 4.61-4.42 (m, 28H), 4.20 (dd, J=16.7, 10.4 Hz, 20H), 3.77-3.50 (m, 22H), 3.41 (d, J=3.9 Hz, 10H), 3.31 (dd, J=19.9, 11.1 Hz, 10H), 2.70 (s, 27H), 1.62 (s, 79H); ¹⁹F NMR (376 MHz, dmso) δ −72.80, −72.82, −72.85, −74.94; MS [M+H]=515.12

### Compound 484

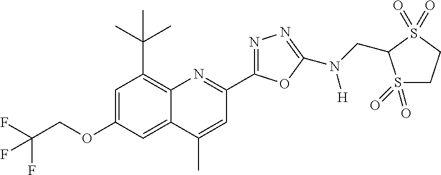

¹H NMR (400 MHz, dmso) δ 8.73 (s, 1H), 7.95 (s, 1H), 7.42 (d, J=2.7 Hz, 1H), 7.33 (s, 1H), 4.96 (dd, J=18.0, 8.9 Hz, 4H), 4.23-4.04 (m, 2H), 4.00 (dd, J=14.9, 6.7 Hz, 2H), 3.94-3.84 (m, 2H), 2.70 (s, 3H), 1.62 (s, 9H); ¹⁹F NMR (376 MHz, dmso) δ −72.80, −72.82, −72.84, −74.57; MS [M+H]=563.23.

### Compound 485

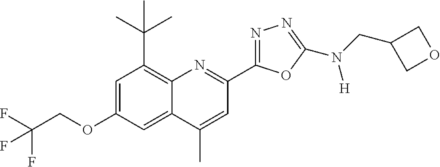

¹H NMR (400 MHz, dmso) δ 7.97 (s, 1H), 7.47 (d, J=2.6 Hz, 1H), 7.39 (d, J=2.7 Hz, 1H), 5.00 (q, J=8.8 Hz, 2H), 4.24 (dd, J=12.2, 5.1 Hz, 1H), 3.95 (dd, J=12.1, 8.2 Hz, 1H), 3.67 (dd, J=12.9, 3.5 Hz, 1H), 3.56 (d, J=6.0 Hz, 2H), 3.40 (dd, J=12.6, 8.5 Hz, 1H), 2.74 (s, 3H), 1.62 (s, 9H); ¹⁹F NMR (376 MHz, dmso) δ −72.77, −72.80, −72.82, −74.43; MS [M+H]=451.29

### Compound 486

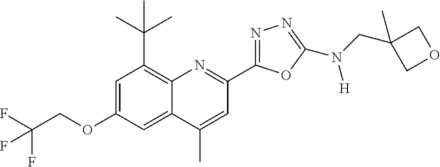

¹H NMR (400 MHz, dmso) δ 7.97 (s, 1H), 7.47 (d, J=2.7 Hz, 1H), 7.39 (d, J=2.6 Hz, 1H), 5.00 (q, J=8.7 Hz, 2H), 4.07 (d, J=12.1 Hz, 1H), 3.86 (d, J=11.9 Hz, 1H), 3.50 (d, J=12.5 Hz, 2H), 3.27 (d, J=12.7 Hz, 1H), 2.74 (s, 3H), 1.62 (s, 9H), 1.07 (s, 3H); ¹⁹F NMR (376 MHz, dmso) δ −72.77, −72.80, −72.82, −74.40; M+1=465.34.

### Compound 487

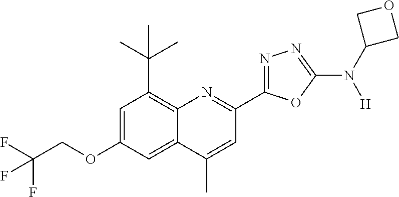

¹H NMR (400 MHz, dmso) δ 8.76 (d, J=5.1 Hz, 1H), δ 7.92 (s, 1H), 7.41 (d, J=2.7 Hz, 1H), 7.32 (d, J=2.7 Hz, 1H), 4.96 (q, J=8.8 Hz, 2H), 4.78 (d, J=2.4 Hz, 3H), 4.58 (s, 2H), 2.69 (s, 3H), 1.62 (s, 9H); ¹⁹F NMR (376 MHz, dmso) δ −72.80, −72.83, −72.85, −75.19; MS [M+H]=437.22

### Compound 488

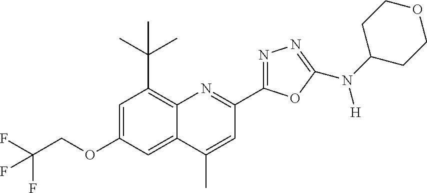

¹H NMR (400 MHz, dmso) δ 8.04 (d, J=7.1 Hz, 1H), 7.92 (s, 1H), 7.41 (d, J=2.7 Hz, 1H), 7.31 (d, J=2.7 Hz, 1H), 4.95 (q, J=8.9 Hz, 2H), 3.87 (d, J=11.4 Hz, 2H), 3.66 (dd, J=10.8, 4.1 Hz, 1H), 3.37 (dd, J=11.5, 9.7 Hz, 2H), 2.69 (s, 3H), 1.93 (d, J=12.5 Hz, 2H), 1.68-1.47 (m, 11H); ¹⁹F NMR (376 MHz, dmso) δ −72.81, −72.83, −72.85; MS [M+H]=465.21

### Compound 489 (enantiomer)

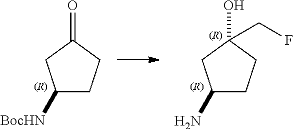

### Step 1

Staring from (R)-tert-butyl 3-oxocyclopentylcarbamate by the procedure of example compound 467.

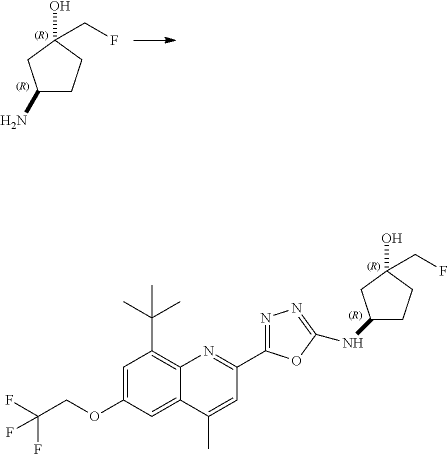

### Step 2

Compound 489 was prepared by analogous procedure of example compound 448.

¹H-NMR (400 MHz, DMSO) δ 8.08 (d, J=6 Hz, 1H), 7.92 (s, 1H), 7.40 (s, 1H), 7.31 (s, 1H), 4.94 (q, J=9 Hz, 2H), 4.20 (d, J=48 Hz, 2H), 3.92 (quin, J=8 Hz, 1H), 2.69 (s, 3H), 2.21 (m, 2H), 2.03 (m, 1H), 1.85 (m, 1H), 1.66 (m, 2H), 1.64 (s, 9H); ¹⁹F NMR (376.1 MHz) δ −72.82 (t, J=9 Hz), −221.51 (s); MS [M−H]⁺=497.14.

### Compound 490 (enantiomer)

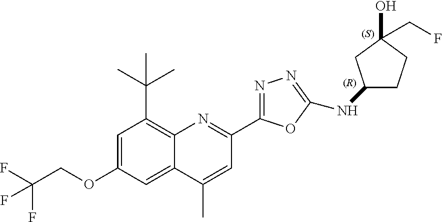

¹H-NMR (400 MHz, DMSO) δ 8.03 (d, J=7 Hz, 1H), 7.40 (d, J=2 Hz, 1H), 7.31 (d, J=2 Hz, 2H), 4.97 (q, J=9 Hz, 2H), 4.86 (s, 1H), 4.21 (d, J=51 Hz, 2H), 2.69 (s, 3H), 2.22-2.15 (m, 1H), 2.05-2.00 (m, 1H), 1.87-1.81 (m, 1H), 1.70-1.52 (m, 3H), 1.61 s, 9H); ¹⁹F NMR (376.1 MHz) δ −72.81 (t, J=9 Hz), −221.19 (t, J=51 Hz); MS [M−H]⁺=497.17.

### Compound 491 (enantiomer)

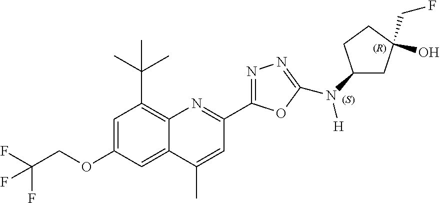

¹H NMR (400 MHz, dmso) δ 8.10 (d, J=6.6 Hz, 1H), 7.92 (s, 1H), 7.40 (d, J=2.6 Hz, 1H), 7.31 (d, J=2.8 Hz, 1H), 4.96 (p, J=8.9 Hz, 3H), 4.26 (s, 1H), 4.14 (s, 1H), 3.92 (dd, J=14.5, 7.6 Hz, 1H), 2.69 (s, 3H), 2.21 (dd, J=13.8, 8.1 Hz, 1H), 2.02 (s, 1H), 1.91-1.77 (m, 1H), 1.64 (d, J=18.8 Hz, 13H); ¹⁹F NMR (376 MHz, dmso) δ −72.80, −72.82, −72.84, −221.37, −221.50, −221.63; MS [M+H]⁺=497.18

### Compound 492 (enantiomer)

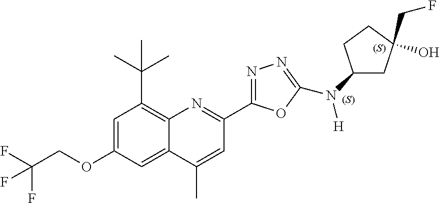

¹H NMR (400 MHz, dmso) δ 8.04 (d, J=6.7 Hz, 6H), 7.92 (s, 6H), 7.40 (s, 6H), 7.31 (d, J=2.7 Hz, 6H), 4.95 (t, J=8.8 Hz, 13H), 4.86 (s, 6H), 4.40-4.06 (m, 20H), 3.56 (d, J=6.6 Hz, 15H), 2.69 (s, 19H), 2.15 (s, 3H), 2.01 (d, J=7.1 Hz, 5H), 1.84 (s, 4H), 1.71 (dd, J=16.0, 9.4 Hz, 21H), 1.61 (s, 65H), 1.32 (s, 7H); ¹⁹F NMR (376 MHz, dmso) δ −72.80, −72.82, −72.85; MS [M+H]⁺=497.17.

### Compound 493

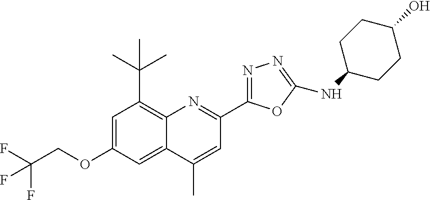

This compound was made analogously to 448 employing an appropriate amine.

¹H NMR (400 MHz, dmso) δ 7.93 (s, 1H), 7.90 (s, 1H), 7.40 (d, J=2.4 Hz, 1H), 7.31 (d, J=2.6 Hz, 1H), 4.95 (q, J=8.8 Hz, 2H), 2.68 (s, 3H), 1.99 (d, J=11.0 Hz, 2H), 1.84 (d, J=10.8 Hz, 2H), 1.75 (d, J=8.4 Hz, 2H), 1.61 (s, 9H), 1.40-1.08 (m, 5H); ¹⁹F NMR (376 MHz, dmso) δ −72.81, −72.83; MS [M+H]⁺=479.21.

### Compound 494

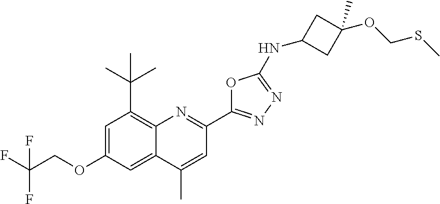

¹H-NMR (400 MHz, CDCl3) δ 7.999 (s, 1H), 7.4 (d, 1H), 7.06 (d, 1H), 4.53 (s, 2H), 4.47 (q, 2H), 4.1 (m, 1H), 2.66 (s, 3H), 2.5 (m, 2H), 2.22 (m, 2H), 2.21 (s, 3H), 1.66 (s, 9H), 1.44 (s, 3H)

¹⁹F NMR (376.1 MHz) δ −74.13 (t)

MS [M+H]⁺=525.21

### Compound 495

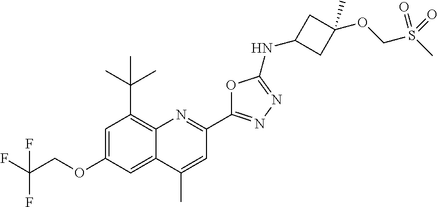

¹H-NMR (400 MHz, CDCl3) δ 8 (s, 1H), 7.39 (d, 1H), 7.06 (d, 1H), 6.05 (br, 1H), 4.48 (q, 2H), 4.45 (m, 2H), 4.1 (br, 1H), 2.93 (s, 1H), 2.67 (s, 3H), 2.63 (m, 2H), 2.44 (m, 2H), 1.65 (s, 9H), 1.45 (s, 3H)

¹⁹F NMR (376.1 MHz) δ −74.11 (t)

MS [M+H]⁺=557.22

### Compound 496

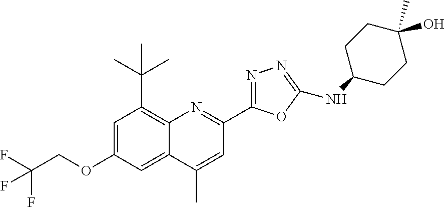

¹H NMR (400 MHz, dmso) δ 7.91 (d, J=6.9 Hz, 2H), 7.40 (s, 1H), 7.31 (s, 1H), 4.95 (d, J=9.0 Hz, 2H), 4.04 (s, 1H), 3.41-3.29 (m, 1H), 2.68 (s, 3H), 1.73 (s, 2H), 1.67 (d, J=13.7 Hz, 2H), 1.61 (s, 10H), 1.55 (s, 2H), 1.34 (s, 1H), 1.10 (s, 3H); ¹⁹F NMR (376 MHz, dmso) δ −72.80, −72.83, −72.85; MS [M+H]⁺=493.18.

### Compound 497

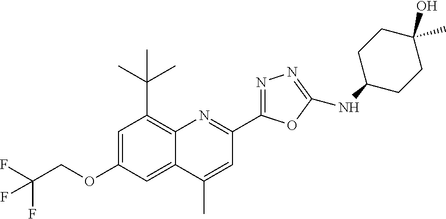

¹H NMR (400 MHz, dmso) δ 7.91 (s, 1H), 7.87 (d, J=7.0 Hz, 1H), 7.40 (d, J=2.5 Hz, 1H), 7.31 (d, J=2.6 Hz, 1H), 4.95 (q, J=8.9 Hz, 2H), 4.24 (s, 1H), 3.58-3.45 (m, 1H), 2.68 (s, 3H), 2.04 (s, 2H), 1.90 (s, 3H), 1.61 (s, 9H), 1.58 (s, 1H), 1.45 (ddd, J=22.0, 20.7, 11.3 Hz, 4H), 1.11 (s, 3H); ¹⁹F NMR (376 MHz, dmso) δ −72.81, −72.83, −72.86; MS [M+H]⁺=493.23.

### Compound 498

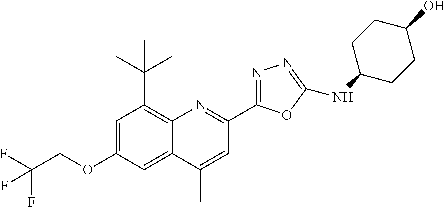

This compound was made analogously to 448 employing an appropriate amine.

¹H NMR (400 MHz, dmso) δ 7.93 (d, J=7.1 Hz, 1H), 7.91 (s, 1H), 7.40 (d, J=2.8 Hz, 1H), 7.31 (d, J=2.7 Hz, 1H), 4.95 (q, J=8.9 Hz, 2H), 4.37 (d, J=2.9 Hz, 1H), 3.70 (s, 1H), 3.49 (s, 1H), 2.69 (s, 3H), 1.84-1.64 (m, 5H), 1.62 (s, 9H), 1.48 (s, 2H); ¹⁹F NMR (376 MHz, dmso) δ −72.80, −72.83, −72.85.

MS [M+H]⁺=479.23.

### Compounds 499-500

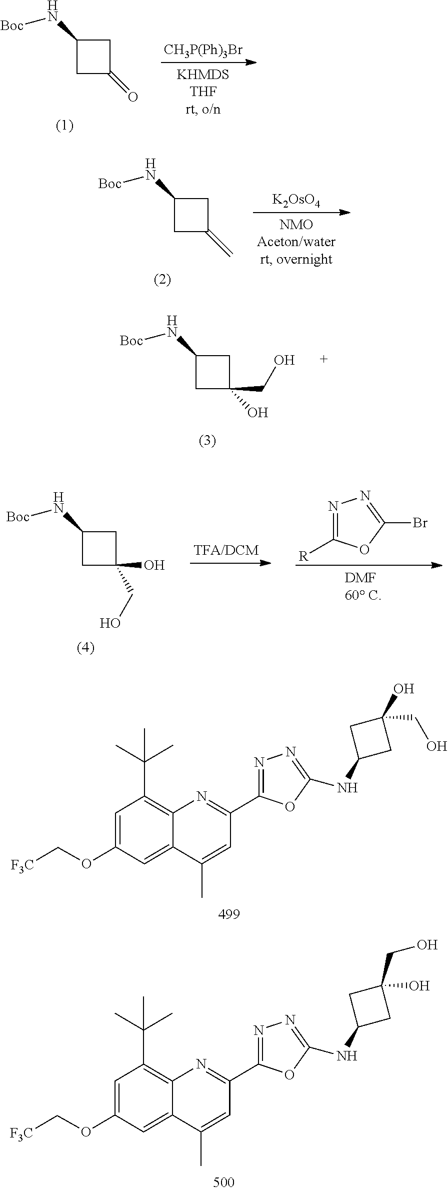

### Step 1

MeP(Ph)₃Br (44.3 g, 121.5 mmol, 1.5 eq) in THF (500 mL) was cooled to −78° C. under N₂ in a three necked flask equipped with additional funnel. KHMDS (0.5M in toluene, 210.6 mL, 103.5 mmol, 1.3 eq) was added to dropwise over 60 mins (internal temperature was monitored below −60° C. After addition, it was stirred for 15 min at −78° C. Tert-butyl 3-oxocyclobutylcarbamate (1) (15 g, 81 mmol, 1 eq) in 300 mL of THF was added slowly while maintaining internal reaction temperature below −60° C. too. The reaction mixture was then left stirred and warmed to RT overnight. Reaction was done by checking on TLC. Diluted with EtOAc, it was washed with sat'd NH₄Cl, brine, dried over Na₂SO₄ then concentrated. The residue was purified by flash chromatography with EA/Hexane to give 8.2 g (2), 55% yield.

### Step 2

The compound (2) (8.2 g, 44.75 mmol, 1 eg) with NMO (10.81 g, 89.5 mmol, 2 eq) in Acetone (240 mL)/water (160 mL) was cooled to 0° C. K₂OsO₄ was added carefully. The mixture was stirred at RT for 18 h. The reaction was monitored by TLC. It was quenched with sat'd Na₂S₂O₃ (500 mL), stirred for 30 mins then concentrated to remove acetone. The aq layer was extracted with EtOAc twice. The organic layers were washed with brine, dried with Na₂SO₄ and purified by fractional re-crystallization from EtOAc/EtOH to give tran-isomer (3) 3.0 g, and cis isomer compound (4) 4.42 g (containing about 10-15% other isomer), 46% yield. Total yield was 84% for both isomers.

Coupling of (3) and (4) with intermediate from example compound 448 produced both 499 and 500.

### Compound 499

¹H-NMR (400 MHz, CD₃OD) δ 7.91 (s, 1H), 7.40 (m, 1H), 7.30 (m, 1H), 4.70 (m, 2H), 4.35 (m, 1H), 3.48 (s, 2H), 2.70 (s, 3H), 2.43 (m, 2H), 2.25 (m, 2H), 1.66 (s, 9H). ¹⁹F NMR (376.1 MHz) δ −76.0 (t); MS [M+H]⁺=481.2

### Compound 500

¹H-NMR (400 MHz, CD₃OD) δ 7.93 (s, 1H), 7.40 (m, 1H), 7.31 (m, 1H), 4.70 (m, 2H), 3.79 (m, 1H), 3.51 (s, 2H), 2.72 (s, 3H), 2.68 (m, 2H), 2.10 (m, 2H), 1.67 (s, 9H). ¹⁹F NMR (376.1 MHz) δ −76.0 (t); MS [M+H]⁺=481.2

### Compounds 501-502

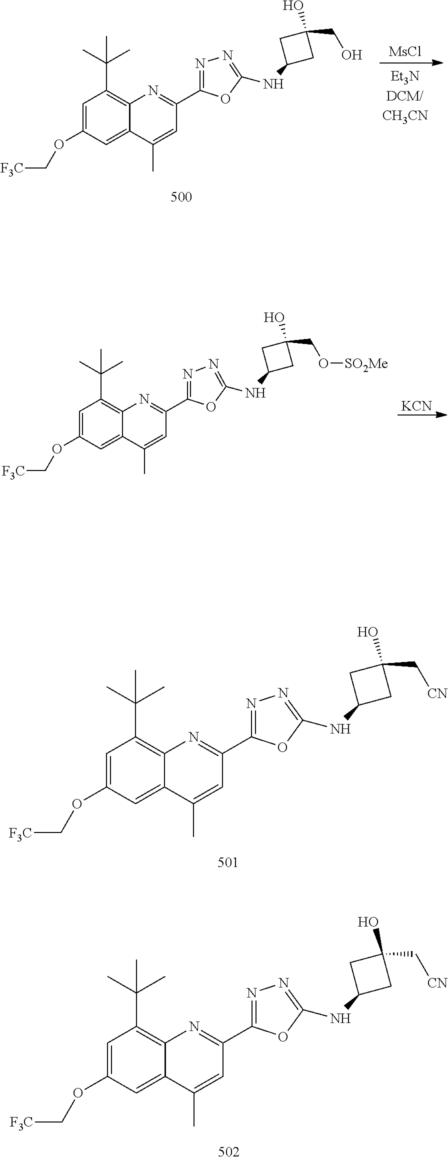

### Step 1

Diol 500 (125 mg, 0.260 mmol) and Et₃N (0.072 mL, 0.52 mmol) in DCM (4 mL) and CH₃CN (1 mL) was cooled to 0° C. under N₂. MsCl (0.024 mL, 0.52 mmol) was added. It was stirred for 30 min at 0° C. Reaction was quenched with a few drops of ice then concentrated. It was purified HPLC to give 34 mg of mono-mesylated compound and 24 mg of bis-mesylated compound.

### Step 2

The mono-Ms compound (34 mg, 0.061 mmol) in 60% EtOAc/water (3 mL) was cooled to 0° C. KCN (12.6 mg, 0.193 mmol) was added carefully. The mixture was stirred at RT for 18 h. Extracted with EtOAc twice, the organic layers were washed with brine, dried with Na₂SO₄ and concentrated to give 27 mg of tran-isomer 501

¹H-NMR (400 MHz, CD₃OD) δ 7.93 (s, 1H), 7.40 (m, 1H), 7.31 (m, 1H), 4.70 (m, 2H), 4.33 (m, 1H), 2.79 (s, 2H), 2.72 (s, 3H), 2.61 (m, 2H), 2.28 (m, 2H), 1.66 (s, 9H). ¹⁹F NMR (376.1 MHz) δ −76.0 (t); MS [M+H]⁺=490.3;

502: It was made with same chemistry from cis-diol 499.

¹H-NMR (400 MHz, CD₃OD) δ 7.93 (s, 1H), 7.40 (m, 1H), 7.31 (m, 1H), 4.70 (m, 2H), 3.49 (m, 1H), 2.79 (s, 2H), 2.72 (s, 3H), 2.60 (m, 2H), 2.26 (m, 2H), 1.64 (s, 9H). ¹⁹F NMR (376.1 MHz) δ −76.0 (t); MS [M+H]⁺=490.3

### Compounds 503-504

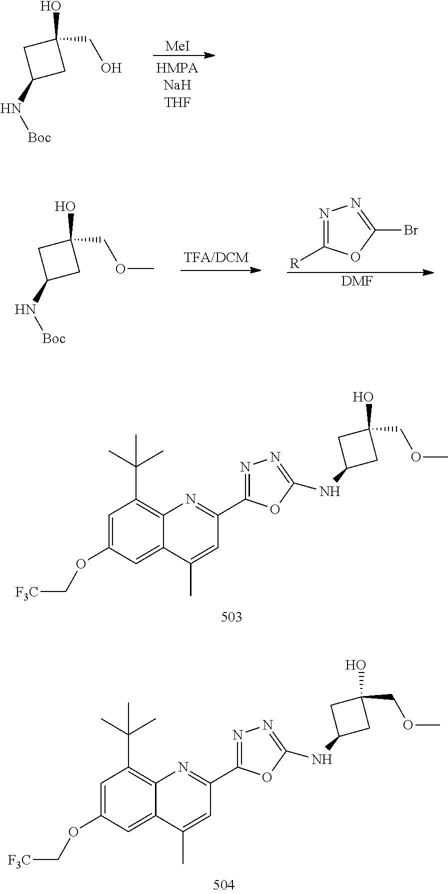

### Step 1

Diol (374 mg, 1.72 mmol) and HMPA (1.8 mL, 10.33 mmol) in THF (4 mL) was added NaH (60% in mineral oil, 83 mg, 2.06 mmol). It was stirred for 60 min at RT then cooled to 0° C. under N₂. MeI (0.314 mL, 2.06 mmol) was added. After the reaction was stirred at rt for 18 h, it was quenched with a few drops of ice then extracted with EtOAc twice. The organic layers were washed with brine, dried with Na₂SO₄ and concentrated. The residue was purified by flash chromatography to give 200 mg mono-methylated compound. It was not clean by

NMR.

### Step 2

The mono-Methylated compound was treated with TFA/DCM the reacted with corresponding bromo-oxadiazol to give 10 mg of cis-isomer 503

Compound 503: ¹H-NMR (400 MHz, CD₃OD) δ 7.93 (s, 1H), 7.42 (m, 1H), 7.33 (m, 1H), 4.70 (m, 2H), 4.27 (m, 1H), 3.65 (s, 2H), 3.28 (s, 3H), 2.62 (s, 3H), 2.60 (m, 2H), 2.13 (m, 2H), 1.68 (s, 9H). ¹⁹F NMR (376.1 MHz) δ −76.8 (t), −78.9 (s);

MS [M+H]⁺=495.2;

Compound 504: It was made with same chemistry on trans-isomer.

¹H-NMR (400 MHz, CD₃OD) δ 7.85 (s, 1H), 7.39 (m, 1H), 7.26 (m, 1H), 4.70 (m, 2H), 4.35 (m, 1H), 3.39 (s, 3H), 3.29 (s, 2H), 2.67 (s, 3H), 2.46 (m, 2H), 2.28 (m, 2H), 1.64 (s, 9H). ¹⁹F NMR (376.1 MHz) δ −75.9 (t), −78.1 (s); MS [M+H]⁺=495.3

### Compounds 505

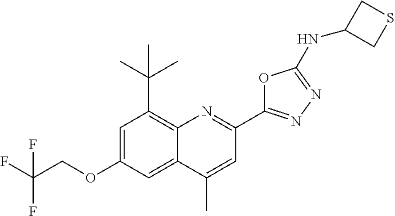

¹H-NMR (400 MHz, CDCl3) δ 8.01 (s, 1H), 7.41 (d, 1H), 7.07 (d, 1H), 6.07 (br., 1H), 5.15 (m., 1H), 4.49 (q, 2H), 3.58 (m, 2H), 3.51 (m, 2H), 2.67 (s, 3H), 1.67 (s, 9H)

¹⁹F NMR (376.1 MHz) δ −74.13 (t)

MS [M+H]⁺=453.26

### Compounds 506

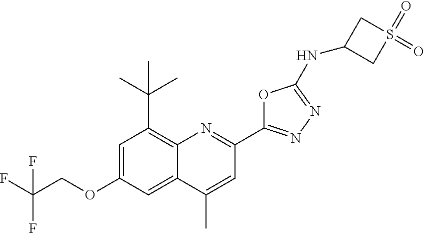

¹H-NMR (400 MHz, CDCl3) δ 8.01 (s, 1H), 7.42 (d, 1H), 7.08 (d, 1H), 6.15 (br., 1H), 4.66 (m, 3H), 4.49 (q, 2H), 4.24 (m, 2H), 2.69 (s, 3H), 1.66 (s, 9H)

¹⁹F NMR (376.1 MHz) δ −74.12 (t)

MS [M+H]⁺=485.17

### Compounds 507

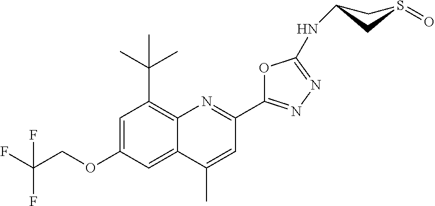

¹H-NMR (400 MHz, CDCl3) δ 8.01 (s, 1H), 7.41 (d, 1H), 7.08 (d, 1H), 4.49 (q, 2H), 4.4 (m, 1H), 4.28 (m, 2H), 3.43 (m, 2H), 2.68 (s, 3H), 1.66 (s, 9H)

¹⁹F NMR (376.1 MHz) δ −74.11 (t)

MS [M+H]⁺=469.15

### Compounds 508

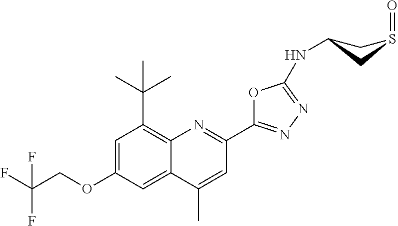

¹H-NMR (400 MHz, CDCl3) δ 8.01 (s, 1H), 7.42 (d, 1H), 7.08 (d, 1H), 6.18 (br., 1H), 4.49 (q, 2H), 4.4 (m, 1H), 3.96 (m, 2H), 3.54 (m, 2H), 2.68 (s, 3H), 1.66 (s, 9H)

¹⁹F NMR (376.1 MHz) δ −74.12 (t)

MS [M+H]⁺=469.14

### Compounds 509

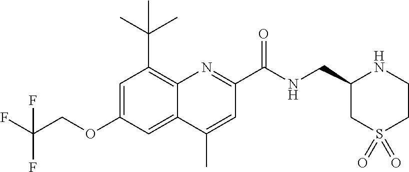

Compound 509 was prepared from intermediate Y and amine used in example compound 416.

¹H-NMR (400 MHz, MeOD) δ 7.99 (s, 1H), 7.44 (d, 1H), 7.34 (d, 1H), 4.73 (q, 2H), 3.67 (m, 1H), 3.49 (m, 2H), 3.16 (m, 2H), 2.88 (m, 1H), 2.74 (s, 3H), 1.68 (s, 9H)

¹⁹F NMR (376.1 MHz) δ −75.98 (t)

MS [M+H]⁺=488.2

### Compounds 510

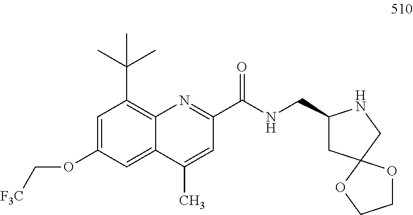

Compound 510 was prepared from intermediate Y and amine used in example compound 443.

¹H-NMR (400 MHz, CDCl₃) δ 8.41 (m, 1H), 8.01 (s, 1H), 7.42 (m, 1H), 7.08 (d, 1H), 4.48 (q, 2H), 3.90 (m, 4H), 3.65-3.51 (m, 2H), 3.00 (q, 2H), 2.69 (s, 3H), 2.11 (m, 1H), 1.99 (b, 1H), 1.78 (m, 1H), 1.66 (s, 9H); ¹⁹F NMR (376.1 MHz) δ −74.12 (t); MS [M+H]⁺=482.2

### Compounds 511

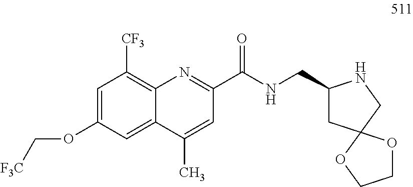

¹H-NMR (400 MHz, CDCl₃) δ 8.51 (m, 1H), 8.19 (s, 1H), 7.83 (d, 1H), 7.43 (d, 1H), 4.54 (q, 2H), 3.91 (m, 4H), 3.62 (m, 2H), 3.56 (m, 1H), 3.00 (q, 2H), 2.74 (s, 3H), 2.58 (m, 2H), 2.13 (m, 1H), 1.79 (m, 1H); ¹⁹F NMR (376.1 MHz) δ −60.75 (s), −74.06 (t); MS [M+H]⁺=494.2

### Compounds 512

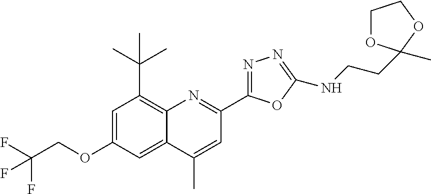

This compound was prepared analogously to example compound 448.

¹H NMR (400 MHz, dmso) δ 7.97 (t, J=5.7 Hz, 1H), 7.91 (s, 1H), 7.41 (d, J=2.6 Hz, 1H), 7.31 (d, J=2.7 Hz, 1H), 4.95 (q, J=8.9 Hz, 2H), 3.82 (m, 4H), 3.25 (d, J=5.7 Hz, 3H), 2.69 (s, 3H), 1.62 (s, 9H), 1.20 (s, 3H); ¹⁹F NMR (376 MHz, dmso) δ −72.81, −72.83, −72.85; MS [M+H]⁺=509.06.

### Compounds 513-517

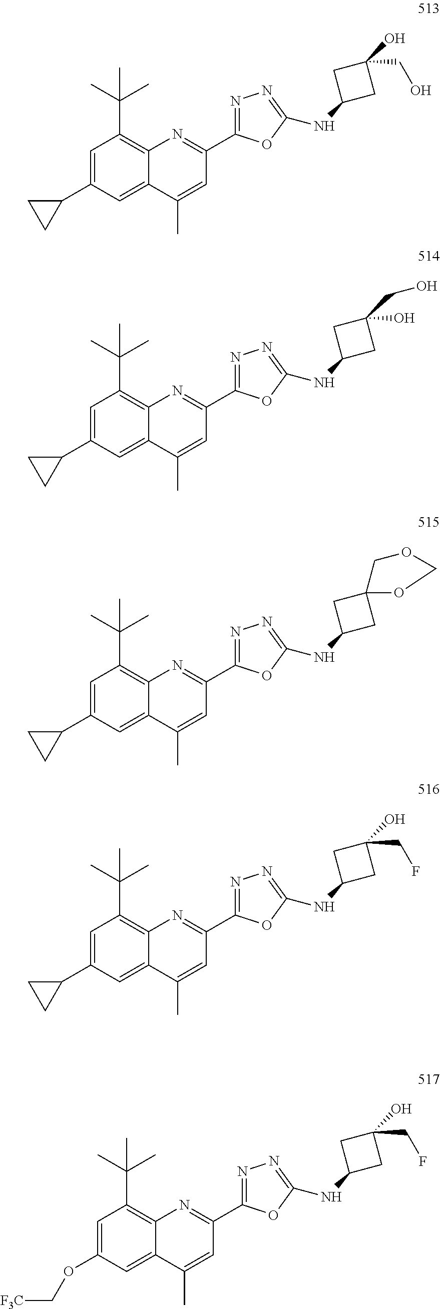

### Compound 513

¹H-NMR (400 MHz, DMSO-d₆) δ 8.25 (m, 1H), 7.85 (s, 1H), 7.56 (s, 1H), 7.38 (s, 1H), 3.66 (m, 1H), 3.56 (m, 1H), 3.24 (s, 2H), 2.67 (s, 3H), 2.51 (m, 2H), 2.14 (m, 1H), 1.96 (m, 1H), 1.62 (s, 9H), 1.03 (m, 2H), 0.83 (m, 2H), ¹⁹F NMR (376.1 MHz) δ −75.02 (d); MS [M+H]⁺=423.2

### Compound 514

¹H-NMR (400 MHz, DMSO-d₆) δ 8.19 (m, 1H), 7.85 (s, 1H), 7.56 (s, 1H), 7.34 (s, 1H), 4.20 (m, 1H), 3.24 (s, 2H), 2.67 (s, 3H), 2.14 (m, 5H), 1.62 (s, 9H), 1.03 (m, 2H), 0.83 (m, 2H), ¹⁹F NMR (376.1 MHz) δ −75.2 (d); MS [M+H]⁺=423.3

### Compound 515

¹H-NMR (400 MHz, CD₃OD) δ 7.85 (s, 1H), 7.56 (1H), 7.41 (m, 1H), 4.89 (m, 2H), 4.83 (m, 1H), 3.89-3.77 (m, 3H), 2.76-2.06 (m, 4H), 2.67 (s, 3H), 1.62 (s, 9H), 1.03 (m, 2H), 0.86 (m, 2H), ¹⁹F NMR (376.1 MHz) δ −75.02 (d); MS [M+H]⁺=435.5

### Compound 516

¹H-NMR (400 MHz, CD₃OD) δ 7.88 (s, 1H), 7.60 (m, 1H), 7.44 (m, 1H), 5.47 (s, 2H), 4.40 (m, 1H), 4.37, 4.25 (d, 2H), 2.70 (s, 3H), 2.50 (m, 2H), 2.40 (m, 2H), 2.12 (m, 1H), 1.67 (s, 9H), 1.10 (m, 2H), 0.83 (m, 2H). ¹⁹F NMR (376.1 MHz) δ −78.06 (s); MS [M+H]⁺=425.3

### Compound 517

¹H-NMR (400 MHz, CD₃OD) δ 7.91 (s, 1H), 7.41 (m, 1H), 7.30 (m, 1H), 4.70 (m, 2H), 4.40 (m, 1H), 4.35, 4.23 (d, 2H), 2.70 (s, 3H), 2.49 (m, 2H), 2.30 (m, 2H), 1.66 (s, 9H). ¹⁹F NMR (376.1 MHz) δ −76.0, −78.1 (d), −228.4 (t); MS [M+H]⁺=483.3

### Compounds 518

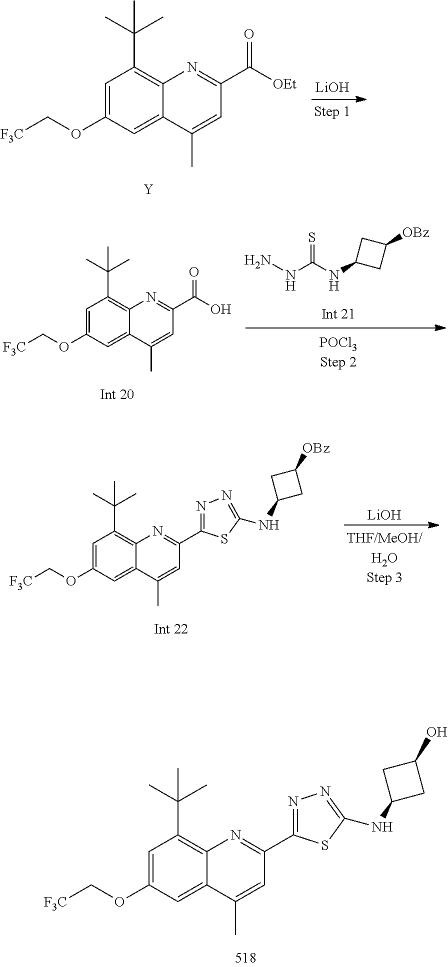

### Step 1:

Intermediate Y in example compound 448 (120 mg, 0.325 mmol) dissolved in THF (500 μL) and MeOH (250 μL) was treated with LiOH (41 mg, 0.976 mmol) dissolved in water. The reaction mixture was stirred at rt for 1 h. After concentrating to dryness, the residue was suspended in EtOAc and washed with 1N HCl soln. The organic layer was concentrated to give Int 20 as a tan solid (86 mg, 77%).

### Step 2:

Int 20 (50 mg, 0.147 mmol) and Int 21 (32 mg, 0.191 mmol) were combined and then phosphorus oxychloride (1.2 mL) was added. The reaction mixture was heated at 70° C. for 1 h. It was then poured into ice water and then extracted with DCM. The organic layer was concentrated to give Int 22.

### Step 3:

The procedures described previously were followed to give Compound 518 as an off-white solid (32 mg, 78%).

¹H NMR (400 MHz, DMSO-d6) δ 8.32-8.29 (m, 1H), 7.97 (s, 1H), 7.39 (s, 1H), 7.30 (s, 1H), 4.95 (d, J=9.2 Hz, 2H), 3.88-3.81 (m, 1H), 3.72-3.63 (m, 1H), 2.69 (s, 3H), 2.67-2.61 (m, 2H), 1.78 (s, 2H), 1.59 (s, 9H); ¹⁹F NMR (376.1 MHz) δ −72.82, −74.97 (TFA salt); MS [M+H]⁺=467.3; LC/MS RT=2.38 min.

### Compounds 519

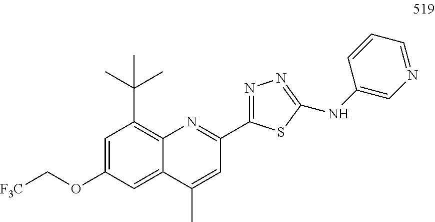

The compound in the example was made according to procedures described previously.

¹H NMR (400 MHz, DMSO-d6) δ 8.32-8.29 (m, 1H), 7.97 (s, 1H), 7.39 (s, 1H), 7.30 (s, 1H), 4.95 (d, J=9.2 Hz, 2H), 3.88-3.81 (m, 1H), 3.72-3.63 (m, 1H), 2.69 (s, 3H), 2.67-2.61 (m, 2H), 1.78 (s, 2H), 1.59 (s, 9H); ¹⁹F NMR (376.1 MHz) δ −72.82, −74.97 (TFA salt); MS [M+H]⁺=467.3; LC/MS RT=2.38 min.

### Compound 520

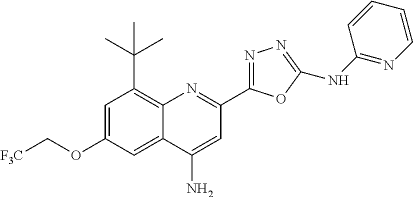

The compound in the example was made according to procedures described previously.

¹H NMR (400 MHz, DMSO-d6) δ 8.26 (s, 1H), 7.84 (s, 2H), 7.56 (s, 1H), 7.24 (d, J=5.8 Hz, 2H), 7.02 (s, 1H), 4.86-4.76 (m, 3H), 1.62 (s, 9H); ¹⁹F NMR (376.1 MHz) δ −72.92, −75.09 (TFA salt); MS [M+H]⁺=459.3; LC/MS RT=2.44 min.

### Compound 521

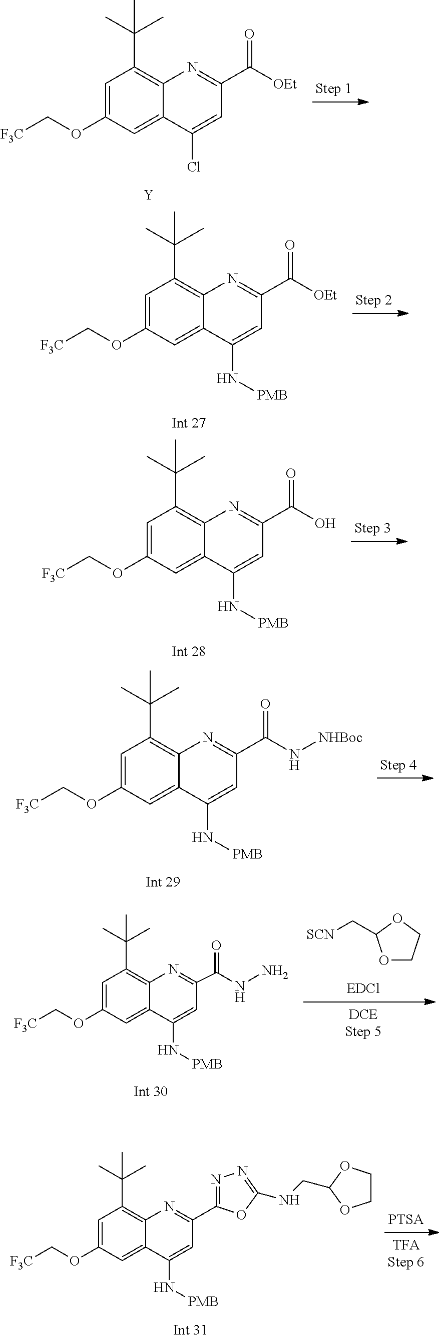

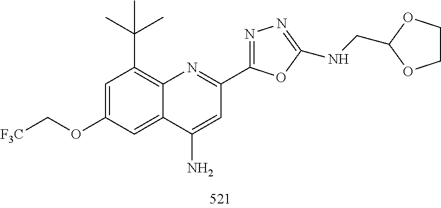

### Step 1:

Int 27 was made according to procedures described in Step 1 of Example 26.

### Step 2:

Int 28 was made according to procedures described in Step 1 of Example 30.

### Step 3:

Int 29 was made according to procedures described in Step 2 of Example 30.

### Step 4:

Int 30 was made according to procedures described in Step 3 of Example 30.

### Step 5:

Int 31 was made according to procedures described previously.

### Step 6:

The procedures described previously were followed to give Compound 521 as a yellow solid.

¹H NMR (400 MHz, DMSO-d6) δ 8.17 (d, J=6.3 Hz, 1H), 7.85 (s, 1H), 7.40 (s, 1H), 7.31 (s, 1H), 5.04 (t, J=4.3 Hz, 1H), 4.96 (q, J=8.9 Hz, 2H), 3.92 (t, J=6.8 Hz, 2H), 3.80 (t, J=6.9 Hz, 2H), 3.41-3.35 (m, 2H), 2.69 (s, 3H), 1.61 (s, 9H); ¹⁹F NMR (376.1 MHz) δ −72.74, −74.85 (TFA salt); MS [M+H]⁺=468.3; LC/MS RT=2.33 min.

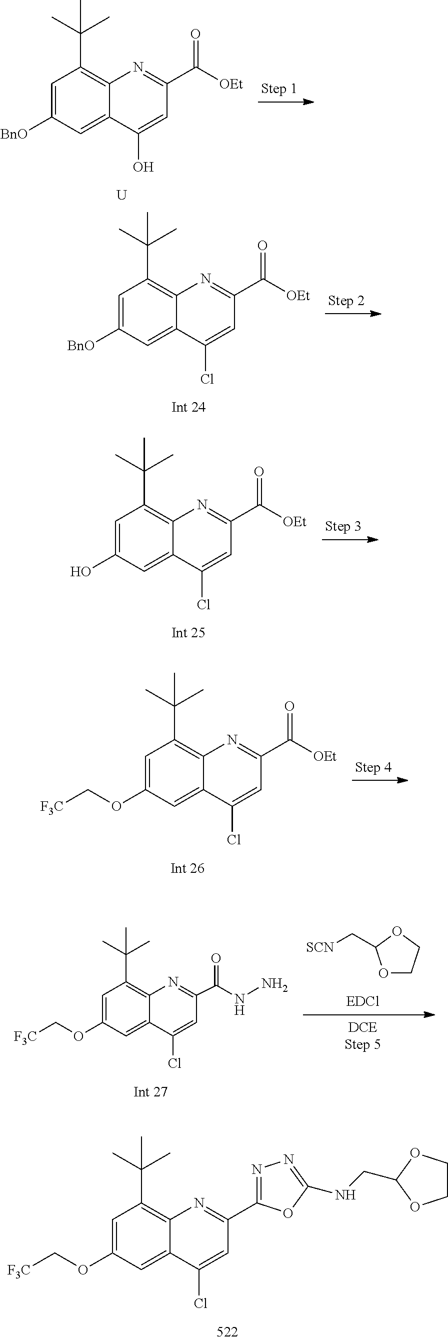

### Step 1:

Intermediate U in example compound 448 (10 g, 26.4 mmol) dissolved in DCE (250 mL) was treated with oxalyl chloride (6.9 mL, 79.2 mL) and a few drops of DMF. The reaction mixture was heated at 55° C. for 1 h. After cooling to rt, the reaction was quenched by adding sat. NaHCO₃ soln and the layers were separated. The organic layer was concentrated to give Int 24 as a yellow solid (10 g, 95%).

### Step 2:

Int 24 (100 mg, 0.252 mmol) dissolved in DCM was cooled to 0° C. and then a solution of 1.0 M BCl3 in DCM (500 μL, 0.504 mmol) was added dropwise. The reaction was stirred at 0° C. for 10 min before warming to rt and stirred for 1 h. The reaction was quenched by adding a solution of triethylamine in MeOH and then concentrated. The residue was redissolved in MeOH and then concentrated again to give Int 25 as a yellow solid (30 mg, 39%).

### Step 3:

Int 26 was made according to procedures described previously.

### Step 4:

Int 27 was made according to procedures described previously.

### Step 5:

Compound 522 was made according to procedures described in previously.

¹H NMR (400 MHz, DMSO-d6) δ 8.32 (s, 1H), 8.16 (s, 1H), 7.53 (s, 1H), 7.42 (s, 1H), 5.02 (m, 3H), 3.92 (t, J=7.0 Hz, 2H), 3.80 (t, J=6.9 Hz, 2H), 3.42-3.37 (m, 2H), 1.61 (s, 9H); ¹⁹F NMR (376.1 MHz) δ −72.74, −74.85 (TFA salt); MS [M+H]⁺=487.3; LC/MS RT=2.49 min.

### Example 47

### Compound 523

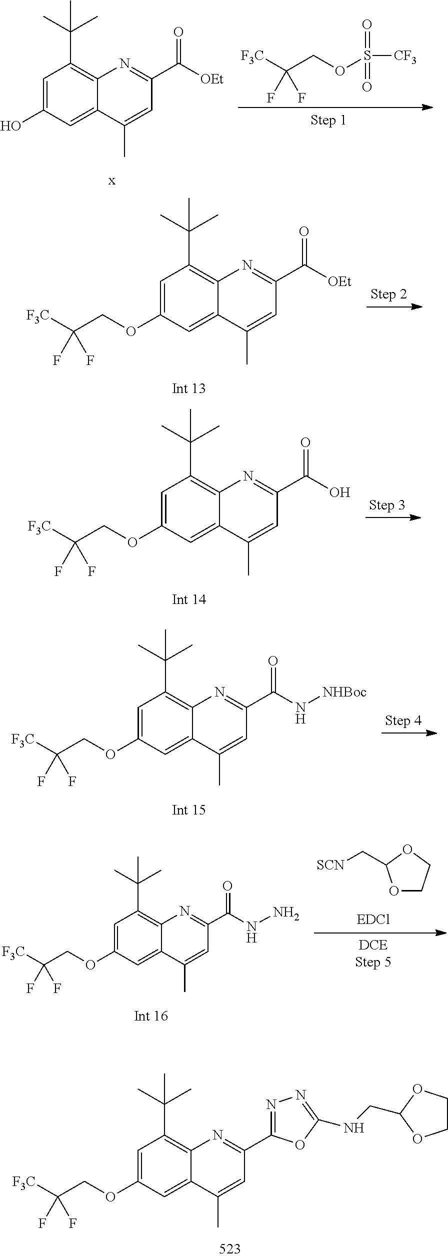

### Step 1:

Int 13 was made according to procedures described in example compound 448.

### Step 2:

Int 14 was made according to procedures described previously.

### Step 3:

Int 15 was made according to procedures described previously.

### Step 4:

Int 16 was made according to procedures described previously.

### Step 5:

The procedures described previously were followed to give compound 523 as an off-white solid.

¹H NMR (400 MHz, DMSO-d6) δ 8.20 (d, J=6.0 Hz, 1H), 7.91 (s, 1H), 7.45 (s, 1H), 7.29 (d, J=2.5 Hz, 1H), 5.03 (dd, J=14.0, 9.5 Hz, 3H), 3.92 (t, J=7.0 Hz, 2H), 3.80 (t, J=6.9 Hz, 2H), 3.42-3.34 (m, 2H), 2.70 (s, 3H), 1.61 (s, 9H); ¹⁹F NMR (376.1 MHz) δ −75.04, −83.03, −122.86 (TFA salt); MS [M+H]⁺=517.2; LC/MS RT=2.50 min.

### Compounds 524-525

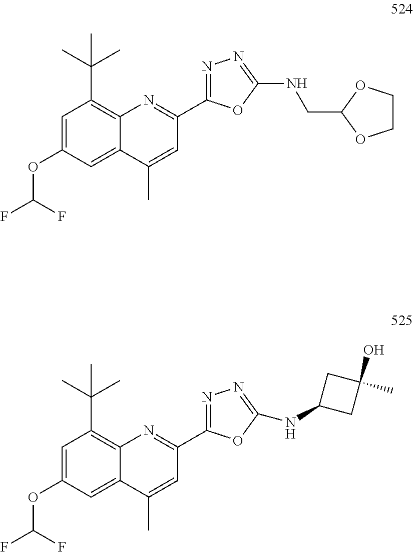

Compound 524 and 525 were prepared in the manner similar to example compound 523.

### Compound 524

¹H-NMR (400 MHz, DMSO-d₆) δ 8.27 (m, 1H), 7.91 (s, 1H), 7.62 (m, 1H), 7.49 (t, 1H), 7.42 (s, 1H), 5.04 (m, 1H), 3.91 (m, 2H), 3.80 (m, 2H), 3.39 (m, 2H), 2.68 (s, 3H), 1.61 (s, 9H). ¹⁹F NMR (376.1 MHz) δ −83.00 (d), −75.17 (s); MS [M+H]⁺=435.2

### Compound 525

¹H-NMR (400 MHz, DMSO-d₆) δ 8.33 (m, 1H), 7.96 (s, 1H), 7.62 (m, 1H), 7.48 (t, 1H), 7.42 (s, 1H), 3.66 (m, 1H), 2.68 (s, 3H), 2.37 (m, 2H), 2.06 (m, 2H), 1.62 (s, 9H), 1.24 (s, 3H), ¹⁹F NMR (376.1 MHz) δ −83.00 (d); MS [M+H]⁺=433.2

### Compound 526

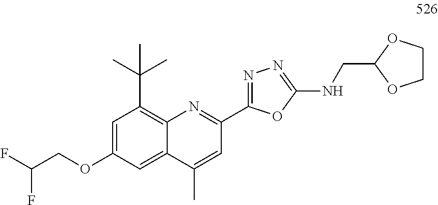

The compound in the example was made according to procedures described previously.

¹H NMR (400 MHz, DMSO-d6) δ 8.19 (d, J=6.2 Hz, 1H), 7.89 (s, 1H), 7.33 (d, J=2.7 Hz, 1H), 7.29 (d, J=2.7 Hz, 1H), 6.46 (t, J=54.4 Hz, 2H), 5.04 (t, J=4.4 Hz, 1H), 4.50 (dd, J=14.6, 11.1 Hz, 2H), 3.96-3.87 (m, 2H), 3.80 (dd, J=8.7, 5.1 Hz, 2H), 3.43-3.34 (m, 2H), 2.69 (s, 3H), 1.61 (s, 9H); ¹⁹F NMR (376.1 MHz) δ −75.24, −126.05 (TFA salt); MS [M+H]⁺=449.2; LC/MS RT=2.45 min.

### Compound 527

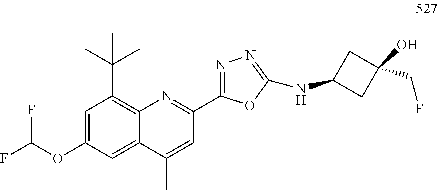

¹H-NMR (400 MHz, CH₃OH-d₄) δ 7.78 (s, 1H), 7.48 (s, 1H), 7.40 (s, 1H), 7.20-6.80 (m, 1H), 4.41 (m, 2H), 3.82 (m, 1H), 2.75 (m, 2H), 2.69 (s, 3H), 2.18 (m, 2H), 1.62 (s, 9H); ¹⁹F NMR (400 MHz, CH₃OH-d₄) δ −84.21 (d, 2F), −228.23 (t, 1F); MS [M+H]⁺=451.

### Compound 528

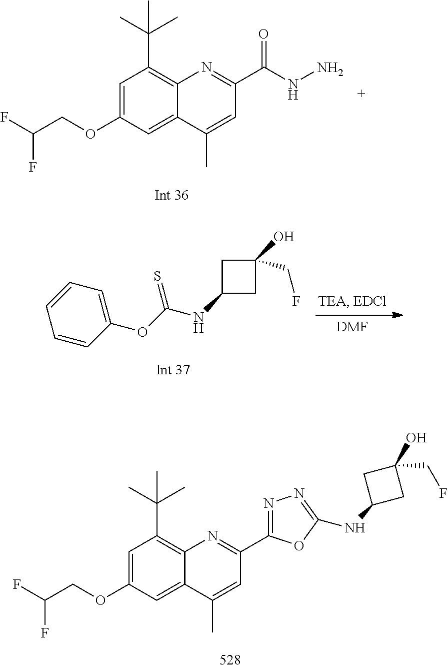

A 100-mL 1-neck rbf was charged with Int 36 (1.4 g, 5.5 mmol), TEA (0.56 g, 5.5 mmol), Int 37 (1.95 g, 5.5 mmol) and DMF (20 mL). The reaction mixture was heated to 65° C. for 30 min. The reaction was cooled back to room temperature. EDCl (1.6 g, 8.2 mmol) was then added. The reaction mixture was heated to 65 C for another 30 min. The reaction mixture was cooled to room temperature and diluted with EtOAc (3000 mL) and washed with water and dried by Na₂SO₄. The organic layer was concentrated and purified by flash chromatography to give Compound 528 as a white solid.

¹H NMR (400 MHz, DMSO-d6) δ 8.33 (d, J=6.1 Hz, 1H), 7.89 (s, 1H), 7.34-7.25 (m, 2H), 6.46 (t, J=54.5 Hz, 1H), 5.49 (s, 1H), 4.50 (dd, J=14.8, 11.3 Hz, 2H), 4.35 (s, 1H), 4.23 (s, 1H), 3.72 (d, J=6.7 Hz, 1H), 2.68 (s, 3H), 2.59-2.51 (m, 2H), 2.07 (s, 2H), 1.61 (s, 9H); ¹⁹F NMR (376.1 MHz) δ −126.12, −225.05; MS [M+H]⁺=465.2; LC/MS RT=2.42 min.

### Compound 529

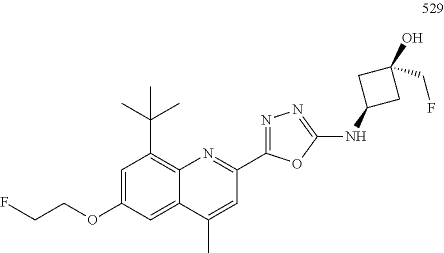

The compound in the example was made according to procedures described previously.

¹H NMR (400 MHz, DMSO-d6) δ 8.32 (d, J=5.9 Hz, 1H), 7.88 (s, 1H), 7.28 (s, 1H), 7.25 (s, 1H), 4.86 (s, 1H), 4.75 (s, 1H), 4.46 (s, 1H), 4.35 (s, 2H), 4.23 (s, 1H), 3.71 (s, 1H), 2.67 (s, 3H), 2.54 (s, 2H), 2.07 (s, 2H), 1.61 (s, 9H); ¹⁹F NMR (376.1 MHz) δ −75.02, −222.30, −225.02 (TFA salt); MS [M+H]⁺=447.2; LC/MS RT=2.25 min.

### Compound 530

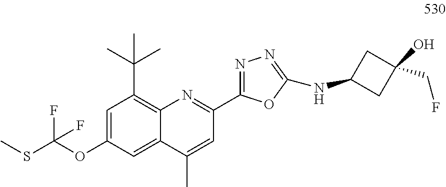

¹H-NMR (400 MHz, CH₃OH-d₄) δ 7.98 (s, 1H), 7.78 (s, 1H), 7.66 (s, 1H), 4.41 (m, 2H), 3.82 (m, 1H), 2.75 (m, 2H), 2.69 (s, 3H), 2.42 (s, 3H), 2.18 (m, 2H), 1.67 (s, 9H); ¹⁹F NMR (400 MHz, CH₃OH-d₄) δ −47.98 (s, 2F), −228.89 (t, 1F); MS [M+H]⁺=497.

### Compound 531

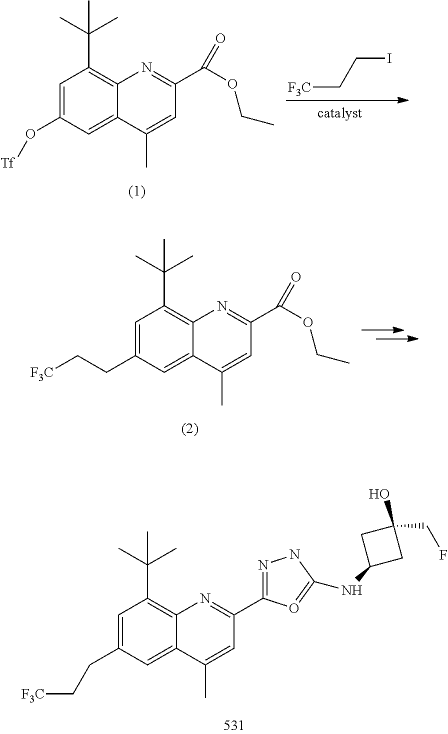

### Step 1

Zn dust (5.84 g, 89.32 mmol) in THF (15 mL) was stirred at rt for 15 min under N₂ then added 1,2-dibromoethane (0.422 mL, 4.89 mmol). Using heating gun to heat up the mixture to reflux for 3 min, then cooled to it in a water bath. Repeating this heating for 3 times total. It was then cooled to 0° C. TMS-Cl (0.662 mL, 5.23 mmol) was added slowly to the mixture. Let it stirred for 5 min at 0° C. and 15 min at it. It was then cooled to 0° C. again. 1,1,1-trifluoro-3-iodopropane (1.09 g, 4.844 mmol) was added carefully. The mixture was stirred at it for another 1 h before diluted with DMA (5 mL) to give organozinc reagent solution (A).

In another reaction flask, ethyl 8-tert-butyl-4-methyl-6-(trifluoromethylsulfonyloxy)quinoline-2-carboxylate prepared from intermediate X from example compound 448 (508 mg, 1.211 mmol) was dissolved in DMA (15 mL) at rt. Dichlorobis-(benzonitrile)palladium (II) (29.3 mg, 0.076 mmol) and 2-dicyclohexy(phosphino-2′-methylbiphenyl (47 mg, 0.128 mmol) were added followed by addition of solution (A). The mixture was heated to 60° C. for 1 h. The reaction was done. It was cooled to rt, diluted with EtOAc, and sat'd NaHCO₃. The mixture was filtered through a pile of Celite. The layers were separated. The organic layer was washed with brine, dried over Na₂SO₄ then concentrated. The residue was purified by flash chromatography with EA/Hexane to give 0.533 g of (2).

Compound 531 was made with same chemistry from (2) as described before.

¹H-NMR (400 MHz, CD₃OD) δ 7.93 (s, 1H), 7.83 (m, 1H), 7.61 (m, 1H), 4.41, 4.30 (d, 2H), 3.83 (m, 1H), 3.08 (m, 2H), 2.73 (m, 2H), 2.67 (s, 3H), 2.56 (m, 2H), 2.13 (m, 2H), 1.68 (s, 9H). ¹⁹F NMR (376.1 MHz) δ −68.4 (s), −229.1 (t); MS [M+H]⁺=481.2

### Compound 532

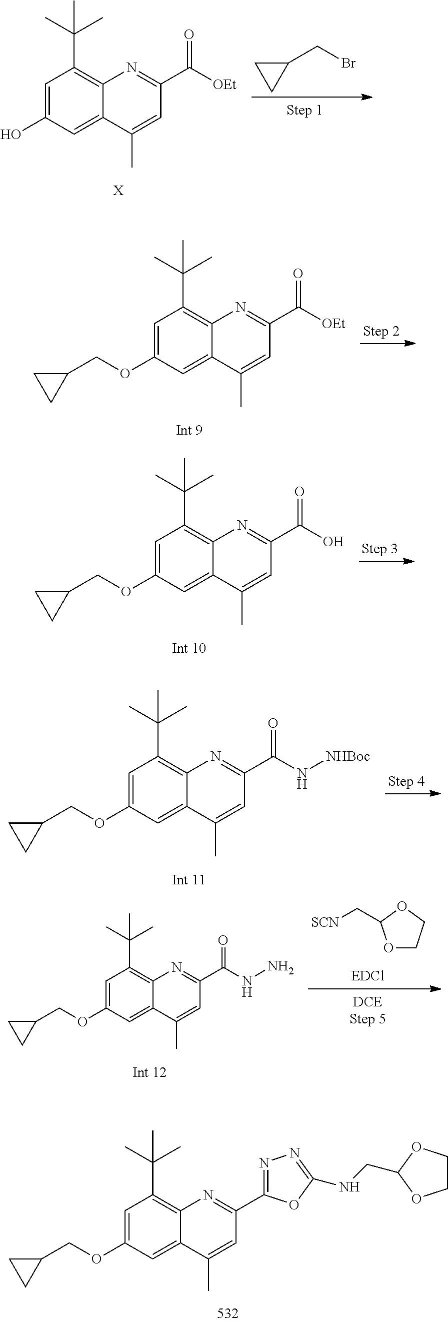

### Step 1:

Int 9 was made according to procedures described previously.

### Step 2:

Int 10 was made according to procedures described in Step 1 of Example 30.

### Step 3:

Int 11 was made according to procedures described in Step 2 of Example 30.

### Step 4:

Int 12 was made according to procedures described in Step 3 of Example 30.

### Step 5:

The procedures described previously were followed to give Compound 532 as a yellow solid.

¹H NMR (400 MHz, DMSO-d6) δ 7.81 (s, 1H), 7.50 (s, 1H), 6.90 (s, 1H), 6.80 (s, 1H), 4.68 (t, J=4.3 Hz, 1H), 3.61 (d, J=7.0 Hz, 2H), 3.56 (t, J=6.9 Hz, 2H), 3.44 (t, J=6.7 Hz, 2H), 3.05-3.00 (m, 3H), 2.29 (s, 3H), 1.25 (s, 9H), 0.92 (s, 1H), 0.24 (d, J=7.8 Hz, 2H); ¹⁹F NMR (376.1 MHz) δ −75.19 (TFA salt); MS [M+H]⁺=439.2; LC/MS RT=2.57 min.

### Compound 533

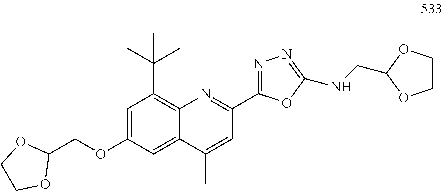

The compound in the example was made according to procedures described previously.

¹H NMR (400 MHz, DMSO-d6) δ 8.19 (t, J=4.5 Hz, 1H), 7.89 (s, 1H), 7.27 (s, 2H), 5.30 (m, 1H), 5.05 (m, 1H), 4.19 (d, J=3.3 Hz, 2H), 3.95 (m, 2H), 3.92 (m, 2H), 3.83 (m, 2H), 3.80 (m, 2H), 3.40 (t, J=4.2 Hz, 2H), 2.69 (s, 3H), 1.62 (s, 9H); ¹⁹F NMR (376.1 MHz) δ −75.06 (TFA salt); MS [M+H]⁺=471.3; LC/MS RT=2.38 min.

### Compound 534

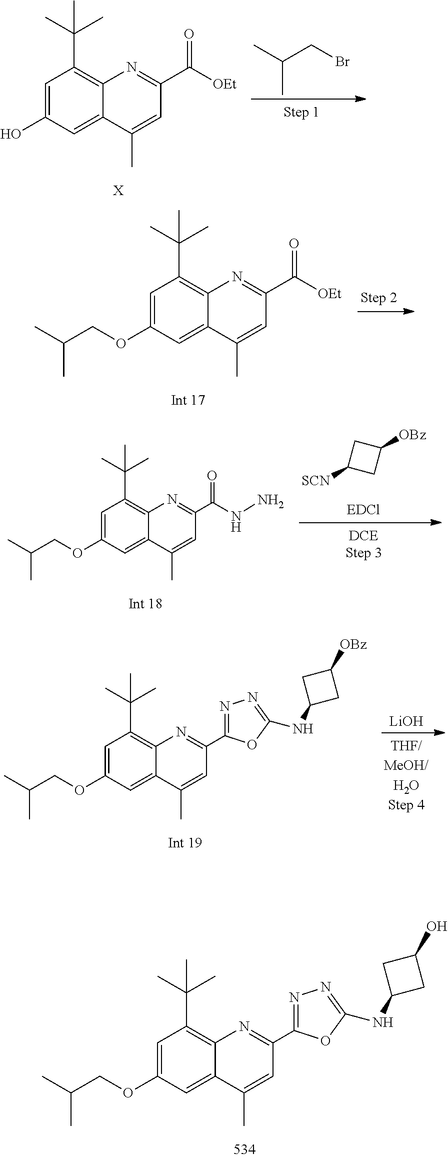

Compound 534 was prepared in the manner described previously.

¹H NMR (400 MHz, DMSO-d6) δ 8.20 (d, J=6.5 Hz, 1H), 7.86 (s, 1H), 7.24 (s, 1H), 7.18 (s, 1H), 3.90 (d, J=6.4 Hz, 2H), 3.83 (m, 1H), 3.55 (m, 1H), 2.66 (s, 3H), 2.65-2.58 (m, 2H), 2.08 (m, 1H), 1.88 (m, 2H), 1.61 (s, 9H), 1.02 (d, J=6.7 Hz, 6H); ¹⁹F NMR (376.1 MHz) δ −75.06 (TFA salt); MS [M+H]⁺=425.2; LC/MS RT=2.45 min.

### Compound 535

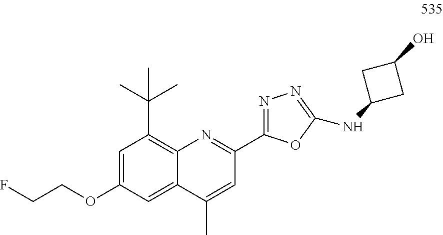

The compound in the example was made according to procedures described previously.

¹H NMR (400 MHz, DMSO-d6) δ 8.21 (d, J=6.5 Hz, 1H), 7.88 (s, 1H), 7.28 (s, 1H), 7.25 (s, 1H), 4.87 (s, 1H), 4.75 (s, 1H), 4.45 (s, 1H), 4.37 (s, 1H), 3.88-3.78 (m, 1H), 3.55 (s, 1H), 2.67 (s, 3H), 2.63 (s, 2H), 1.87 (d, J=11.7 Hz, 2H), 1.61 (s, 9H); ¹⁹F NMR (376.1 MHz) δ −75.03, −222.38 (TFA salt); MS [M+H]⁺=415.2; LC/MS RT=2.29 min.

### Compound 536

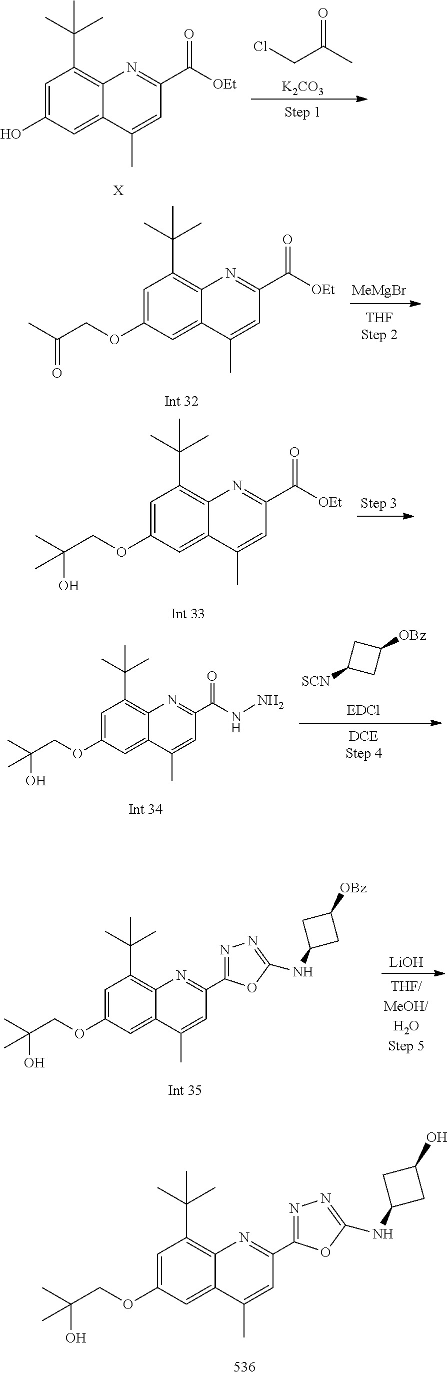

### Step 1:

Int 32 was made according to procedures described in example compound 448.

### Step 2:

Int 32 (70 mg, 0.204 mmol) dissolved in THF (2 mL) was cooled to 0° C. and treated with methyl magnesium bromide (200 μL, 0.612 mmol). The reaction mixture was stirred at rt for 30 min. After reaction had reached completion, the reaction mixture was diluted with EtOAc and washed with 1N HCl soln. The organic layer was concentrated to give Int 33 as a yellow solid (66 mg, 90%).

### Step 3:

Int 34 was made according to procedures described previously.

### Step 4:

Int 35 was made according to procedures described previously.

### Step 5:

Compound 536 was made according to procedures described previously

¹H NMR (400 MHz, DMSO-d6) δ 8.20 (s, 1H), 7.86 (s, 1H), 7.28 (s, 1H), 7.17 (s, 1H), 3.87 (s, 2H), 3.82 (d, J=7.3 Hz, 1H), 3.56 (d, J=7.1 Hz, 1H), 2.66 (s, 3H), 2.63 (s, 2H), 1.87 (d, J=11.2 Hz, 2H), 1.61 (s, 9H), 1.23 (s, 6H); ¹⁹F NMR (376.1 MHz) δ −75.28 (TFA salt); MS [M+H]⁺=441.3; LC/MS RT=2.15 min.

### Compound 537

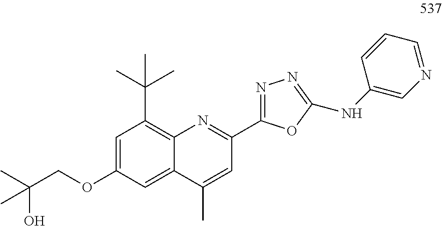

The compound in the example was made according to procedures described previously.

¹H NMR (400 MHz, DMSO-d6) δ 8.89 (s, 1H), 8.32 (s, 1H), 8.27-8.21 (m, 1H), 7.98 (s, 1H), 7.59-7.53 (m, 1H), 7.32 (s, 1H), 7.22 (s, 1H), 3.89 (s, 2H), 2.71 (s, 3H), 1.66 (s, 9H), 1.24 (s, 6H); ¹⁹F NMR (376.1 MHz) δ −74.82 (TFA salt); MS [M+H]⁺=448.3; LC/MS RT=2.20 min.

### Example 48

### Compound 538

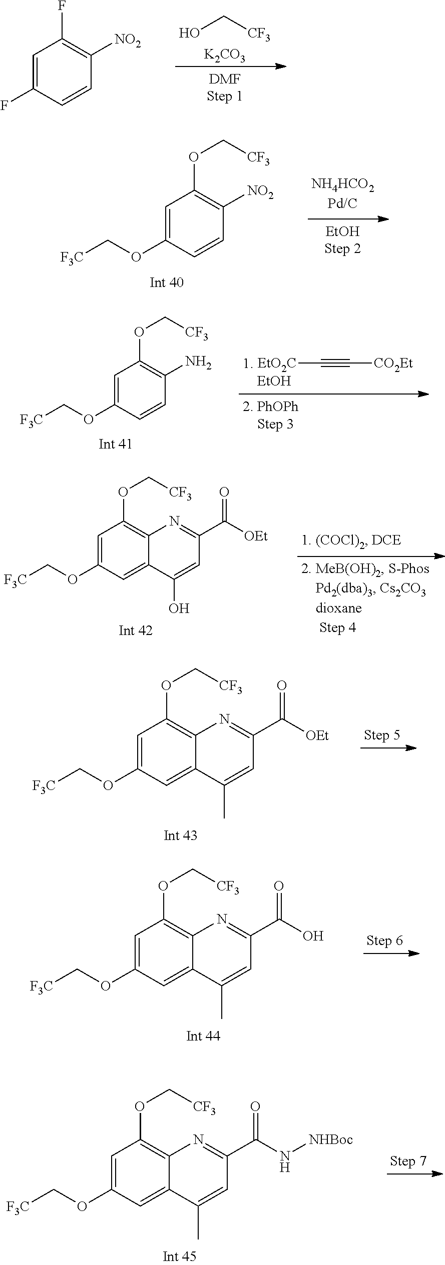

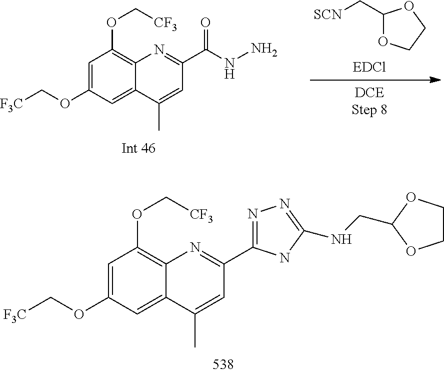

### Step 1:

To a solution of 2,4-difluoronitrobenzene (690 μL, 6.23 mmol) in DMF (12 mL) was added powdered potassium carbonate (2.60 g, 18.9 mmol) followed by trifluoroethanol (900 μL, 12.6 mmol). The reaction mixture was stirred at 80° C. overnight. Water was added to the reaction mixture and the resulting precipitate was filtered and dried to give Int 40 as a yellow solid (1.4 g, 70%).

### Step 2:

Int 40 (1 g, 3.46 mmol) dissolved in EtOH (35 mL) was treated with ammonium formate (1.31 g, 20.8 mmol) followed by 10% Pd/C (370 mg, 0.346 mmol). The reaction mixture was heated at 55° C. for 1.5 h. After cooling to rt, the reaction mixture was filtered and the filtrate was concentrated to give Int 41 as a pale pink solid (750 mg, 83%).

### Step 3:

Int 42 was made according to procedures described in Step 1 and Step 2 of Example 1.

### Step 4:

Int 42 (1.0 g, 2.59 mmol) suspended in DCE (25 mL) was treated with oxalyl chloride (680 μL, 7.77 mmol) and a few drops of DMF. The reaction mixture was heated at 55° C. for 20 min. After cooling to rt, the reaction mixture was quenched by adding sat. NaHCO₃ soln and the layers were separated. The organic layer was concentrated to give an off-white solid.

The solid (980 mg, 2.27 mmol) was dissolved in dioxane and the solution was degassed with N₂ for a few minutes before Pd₂(dba)₃ (52 mg, 0.057 mmol), methyl boronic acid (409 mg, 6.82 mmol), S-Phos (93 mg, 0.227 mmol), and cesium carbonate (2.96 g, 9.09 mmol) were added. The reaction mixture was heated at 100° C. for 2 h. After cooling to rt, the reaction mixture was diluted with EtOAc and washed with Na₂CO₃ soln, water, and brine. The organic layer was concentrated and purified by flash chromatography to give Int 43 as a yellow solid (230 mg, 25%).

### Step 5:

Int 44 was made according to procedures described in Step 1 of Example 30.

### Step 6:

Int 45 was made according to procedures described in Step 2 of Example 30.

### Step 7:

Int 46 was made according to procedures described in Step 3 of Example 30.

### Step 8:

Compound 538 was made according to procedures described previously.

¹H NMR (400 MHz, DMSO-d6) δ 8.24 (s, 1H), 7.98 (s, 1H), 7.21 (d, J=5.5 Hz, 2H), 5.00 (tt, J=17.7, 9.0 Hz, 5H), 3.98-3.88 (m, 2H), 3.80 (t, J=6.7 Hz, 2H), 3.53 (s, 3H), 3.39 (d, J=3.3 Hz, 2H); ¹⁹F NMR (376.1 MHz) δ −72.86, −73.97 (TFA salt); MS [M+H]⁺=509.2; LC/MS RT=2.31 min.

### Compound 539

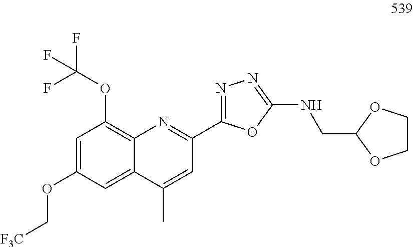

Compound 539 was prepared in the manner similar to example compound 538.

¹H-NMR (400 MHz, CDCl₃) δ 8.15 (s, 1H), 7.38 (s, 1H), 7.18 (m, 1H), 5.31 (m, 1H), 5.13 (m, 1H), 4.51 (m, 2H), 4.10-3.87 (m, 6H), 3.71 (m, 2H), 2.71 (s, 3H). ¹⁹F NMR (376.1 MHz) δ −57.82 (s), −74.06 (t); MS [M+H]⁺=495.2

### Example 49

### Compound 540

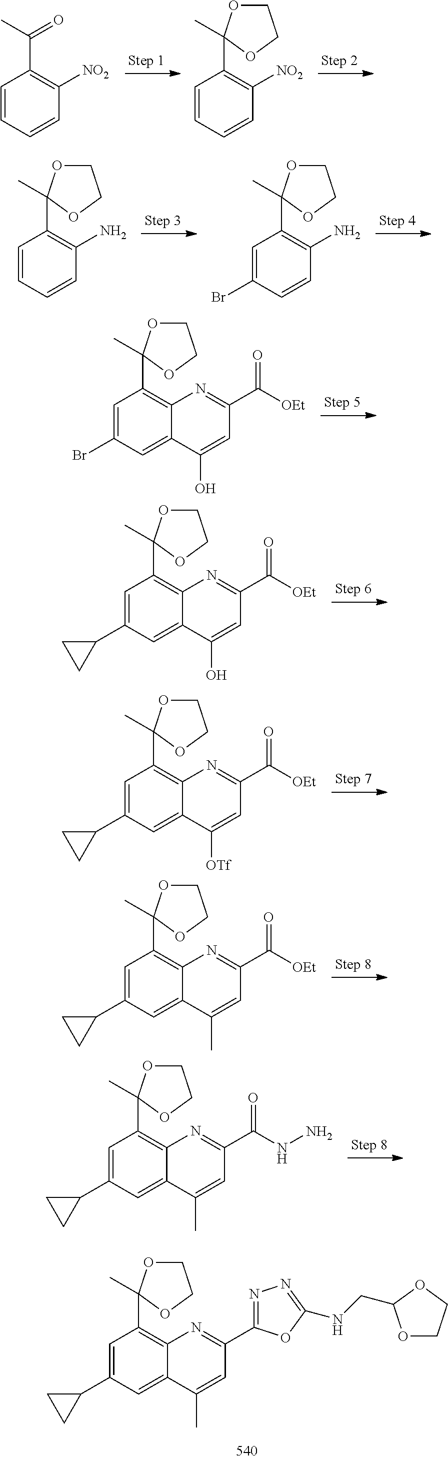

### Step 1

Intermediate 1 was prepared using the method of Kingsbury (Kingsbury, William D.; Boehm, Jeffrey C.; Jakas, Dalia R.; Holden, Kenneth G.; Hecht, Sidney M, Journal of Medicinal Chemistry, 1991, 34:1, 98-107;

### Step 2

Intermediate 1 (24 g, 115 mmol) was taken up in 550 mL of warm ethanol (stirred for 45 min to fully dissolve) and treated with ˜20 mL of a slurry of 2800 Raney Ni, and the mixture stirred vigorously under H₂ at 45° C. After 3 h, the reaction was stirred under vacuum (60 torr) for several minutes, then filtered and concentrated to provide Intermediate 2 (23.4 g, 111% yield) as an off-white solid.

### Step 3

A solution of Intermediate 2 (26 g, 145 mmol) in 350 mL MeCN was cooled to −30° C. and treated with NBS (25.8 g, 145 mmol). The mixture was allowed to warm slowly to 0° C., then a 1N sodium sulfite solution was added and the mixture stirred for 30 min. The reaction was partitioned between 750 mL EtOAc and 750 mL 1N sodium carbonate. The organic layer was washed with 2.5% LiCl and brine, dried with sodium sulfate and filtered thru silica gel to provide intermediate 3 (35.8 g, 96% yield) as a tan solid.

MS [M+H]+=258.04.

### Step 4

A 1-L 3-neck Morton flask was charged with intermediate 3 (37.4 g, 145 mmol) and 250 mL MeOH. Diethylacetylene dicarboxylate (25.4 mL, 160 mmol) was added, and the mixture heated to 65° C. for 1 h. The MeOH was distilled off using a short path distillation apparatus, and the residue heated to 220° C. in a reaction block. Once the internal temperature reached 217° C. the reaction was removed from heating and cooled to an internal temperature of 140° C. Heptane (300 mL was then added, and the mixture left to stir overnight. The residue was purified by silica chromatography to provide the desired product (17.1 g, 31% yield) as a brown solid. MS [M+H]⁺=382.11.

### Step 5

A solution of Intermediate 4 (3.74 g, 9.79 mmol) in 100 mL dioxane was thoroughly degassed and treated with cyclopropylboronic acid (2.95 g, 34.3 mmol), cesium carbonate (11.16 g, 34.3 mmol) and [1,1′-Bis(diphenylphosphino)ferrocene]palladium(II) dichloride (959 mg, 1.17 mmol). After heating at 100 C for 5 min, aqueous workup with ethyl acetate and citric acid, followed by silica gel chromatography provided the desired product (1.82 g, 54% yield) as an off white solid. MS [M+H]⁺=344.14.

### Step 6

A solution of Intermediate 5 (1.85 g, 5.4 mmol) in 25 mL DCM was cooled to 0 C and treated with lutidine (1.56 mL, 13.5 mmol) and Tf2O (1.90 mL, 11.3 mmol). After stirring for 1 h the reaction was diluted with EtOAc and pH 2 phosphate buffer. The organic layer was dried (Na2SO4) and concentrated to provide the desired product (2.5 g, 100%) as a light colored oil. MS [M+H]+=476.11

### Step 7

A solution of Intermediate 6 (2.5 g, 5.4 mmol) in 25 mL dioxane was treated with methylboronic acid (1.134 g, 18.9 mmol), potassium carbonate (2.98 g, 21.6 mmol) and [1,1-Bis(diphenylphosphino)ferrocene]palladium(II) dichloride (441 mg, 0.54 mmol). The mixture was heated at 100 C for 15 min, then cooled to rt, diluted with EtOAc and washed with water and brine. The residue was purified by silica gel chromatography to provide the desired product (1.88 g, 102% yield) as a tan solid. MS [M+H]+=342.15.

### Step 8

A solution of Intermediate 7 (900 mg, 2.64 mmol) was taken up in 24 mL THF, 16 mL MeOH and 6.6 mL 1N LiOH. After 15 min at rt, the reaction was diluted with EtOAc and washed with pH 3 citrate buffer. The organic was washed with brine, dried with sodium sulfate and concentrated. The residue was taken up in 15 mL DCM at 0 C and treated with NMM (0.68 mL, 6.6 mmol). Isobutylchloroformate (415 uL, 3.17 mmol) was added dropwise and the reaction mixture stirred for 15 min, then treated solution of hydrazine (2.5 mL, 7.92 mmol) and TEA (1.06 ml, 7.6 mmol). Aqueos work-up with EtOAc and pH 5 citrate buffer provided the desired product (260 mg, 31% yield) as a tan sold.

MS [M+H]⁺=328.23.

### Step 9

A mixture of intermediate 8 (288 mg, 0.88 mmol) and 2-(isothiocyanatomethyl)-1,3-dioxolane (132 mg, 0.91 mmol) in 10 mL DCE were heated to 60 C for 2 h. The mixture was then cooled to rt and treated with EDCl (510 mg, 2.7 mmol). The mixture was heated at 60 C overnight. The reaction was diluted with EtOAc and washed with 10% citric acid, sodium bicarbonate and brine. Purification by silica chromatography gave the desired product 540 (227 mg, 59% yield) as a tan solid. ¹H-NMR (400 MHz, DMSO) δ 8.24 (t, J=6 Hz, 1H), 7.90 (s, 1H), 7.70 (d, J=2 Hz, 1H), 7.59 (d, J=2 Hz, 1H), 5.04 (t, J=6 Hz, 1H), 3.99-3.91 (m, 4H), 3.81 to 3.78 (m, 2H), 3.74-3.70 (m, 2H), 2.69 (s, 3H), 2.16 (m, 1H), 2.05 (s, 3H), 1.07-1.04 (m, 2H), 0.85-0.81 (m, 2H); MS [M+H]⁺=439.17.

### Compound 541

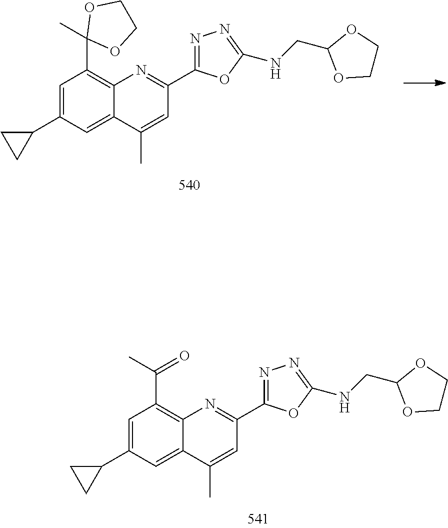

A solution of Compound 540 (150 mg, 0.34 mmol) in 8 mL THF was treated with 1.5 mL 2N HCl and heated to 60° C. for 30 min. The reaction is diluted with ice water and the precipitate is filtered to provide Compound 541 (117 mg, 87% Yield) as a bright yellow solid. ¹H-NMR (400 MHz, DMSO) δ 8.25 (t), 7.91 (s, 1H), 7.69 (s, 1H), 7.56 (s, 1H), 5.03 (t, J=4 Hz, 1H), 3.92 (m, 2H), 3.80 (m, 2H), 3.39 (t, J=5 Hz, 2H), 2.70 (s, 3H), 2.19 (m, 1H), 2.11 (s, 3H), 1.05 (m, 2H), 0.84 (m, 2H; MS [M+H]⁺=395.23.

### Compound 542

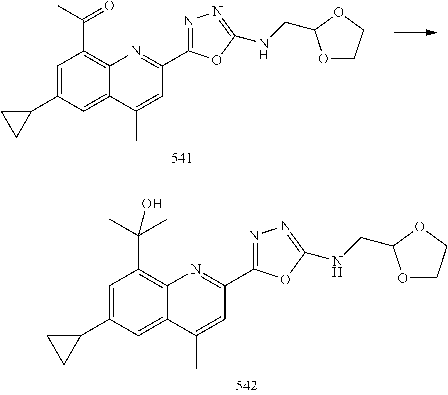

A solution of Compound 541 (25 mg, 0.063 mmol) in 10 mL THF was cooled to 0 C and treated with 150 uL of 3N MeMgBr in diethyl ether. After 5 min the reaction was diluted with EtOAc and water. The organic layer was dried with sodium sulfate, filtered thru silica and concentrated to provide Compound 542 (23.2 mg, 96% Yield) as a yellow solid; ¹H-NMR (400 MHz, DMSO) δ 8.26 (t, 1H), 7.90 (s, 1H), 7.62 (m, 2H), 6.25 (s, 1H), 5.03 (t, J=4 Hz, 1H), 3.91 (m, 2H), 3.81 (m, 2H), 3.40 t, J=4 Hz, 1H), 2.70 (s, 3H), 2.15 (m, 1H), 1.71 (s, 6H), 1.04 (m, 2H), 0.85 (m, 2H); MS [M+H]⁺=411.11.

### Compound 543

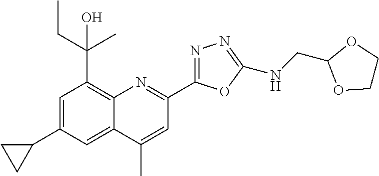

Compound 543 was prepared using a similar method to compound 542

¹H-NMR (400 MHz, MeOD) δ 7.97 (s, 1H), 7.70 (d, J=2 Hz, 1H), 7.48 (d, J=2 Hz, 1H), 5.10 (t, J=4 Hz, 1H), 3.88 (m, 2H), 3.68 (m, 2H), 3.31 (d, J=4 Hz, 2H), 2.07 (m, 2H), 2.02 (m, 1H), 1.09 (m, 2H), 0.86 (m, 2H), 0.762 (t, J=7 Hz, 3H); MS [M+H]⁺=425.13.

### Compound 544

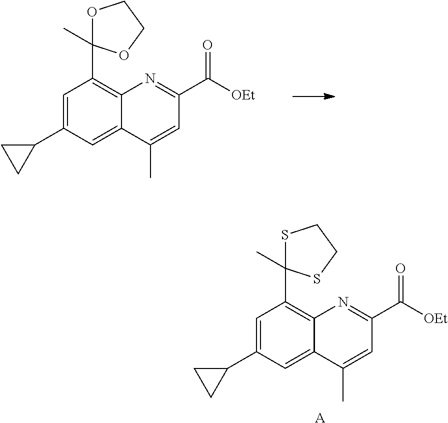

A solution of intermediate in example compound 540 (100 mg, 0.292 mmol) in 350 uL DCE was treated with ethanedithiol (98 uL, 1.168 mmol) and BF₃—OEt₂ (40 uL, 0.321 mmol). The mixture heated at 70° C. for 2 h in a sealed vial. The reaction was diluted with EtOAc and washed with 1N carbonate buffer. The crude product was purified by flash column chromatography to provide compound A (66.4 g, 61% yield) as a white solid; MS [M+H]⁺=374.15.

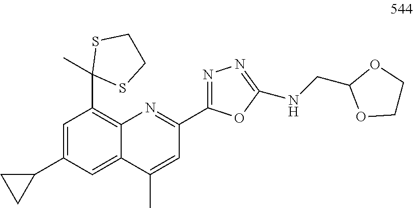

Compound 544 was prepared from Intermediate A using same method used for Compound 540. ¹H-NMR (400 MHz, DMSO) δ 8.22 (t, 1H), 7.95 (s, 1H), 7.91 (s, 1H), 7.66 (s, 1H), 5.06 (t, 1H), 3.92 (m, 2H), 3.81 (m, 2H), 3.40 (m, 2H), 3.33 (m, 2H), 3.15 (m, 2H), 2.69 (s, 3H), 2.39 (s, 3H), 2.15 (m, 1H), 1.05 (m, 2H), 0.82 (m, 2H); MS [M+H]⁺=471.24.

### Compound 545

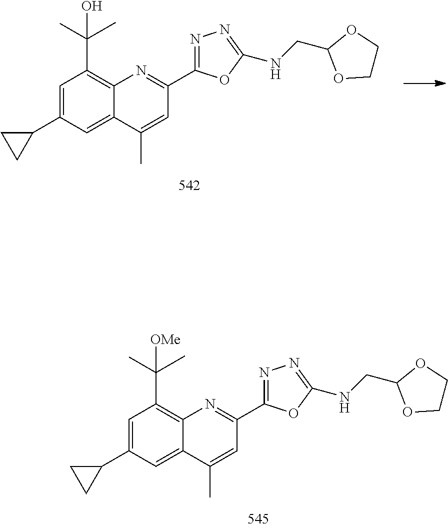

A solution of compound 542 (50 mg, 0.121 mmol) in 5 mL MeOH was treated with 0.5 mL TFA and heated to 55° C. for 90 min. The reaction mixture was concentrated in vacuo and the residue sonicated in Et₂O to provide the desired product 545 (32 mg, 60% yield) as a yellow solid. ¹H-NMR (400 MHz, MeOD) δ 7.89 (s, 1H), 7.69 (s, 1H), 7.63 (s, 1H), 5.11 (m, 1H), 4.04 (m, 2H), 3.90 (m, 2H), 3.55 (m, 2H), 3.38 (s, 3H), 2.72 (s, 3H), 1.91 (s, 6H), 1.10 (m, 2H), 0.86 (m, 2H); MS [M+H]⁺=425.21.

### Compound 546

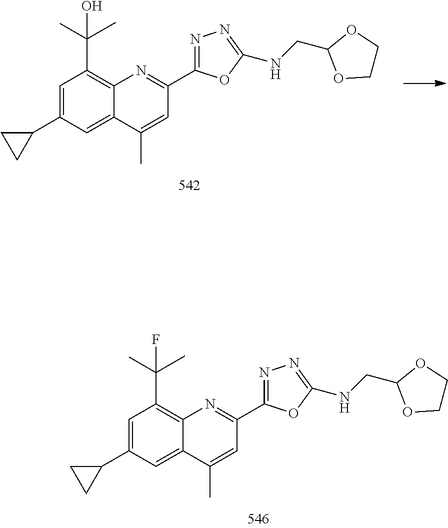

H-NMR (400 MHz, DMSO) δ 8.25 (t, J=6 Hz, 1H), 7.91 (s, 1H), 7.69 (s, 1H), 7.56 (s, 1H), 5.03 (t, J=4 Hz, 1H), 3.92 (m, 2H), 3.80 (m, 2H), 3.39 (t, J=5 Hz, 2H), 2.70 (s, 3H), 2.19 (m, 1H), 2.00 (s, 3H), 1.94 (s, 3H), 1.05 (m, 2H), 0.84 (m, 2H); MS [M+H]⁺=413.19.

### Compound 547

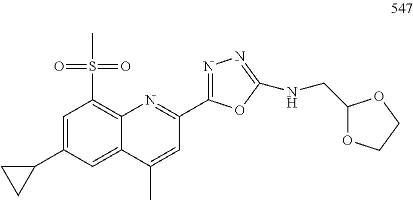

Compound 547 was prepared in the manner similar to example compound 540.

¹H-NMR (400 MHz, CH₃OH-d₄) δ 8.18 (s, 1H), 8.16 (s, 1H), 8.08 (s, 1H), 5.10 (m, 1H), 4.02 (m, 2H), 3.85 (m, 2H), 3.63 (s, 1H), 3.58 (m, 3H), 2.80 (s, 3H), 2.26 (m, 1H), 0.90 (m, 4H); MS [M+H]⁺=431.

### Compound 548

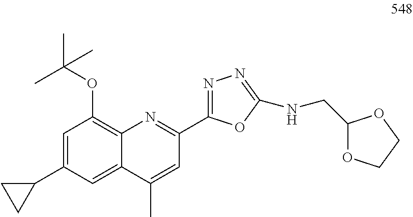

Compound 548 was prepared in the manner similar to example compound 540.

¹H-NMR (400 MHz, MeOD) δ 7.94 (s, 1H), 7.54 (d, 1H), 7.12 (d, 1H), 5.1 (m, 1H), 4 (m, 2H), 3.88 (m, 2H), 3.58 (m, 2H), 2.74 (s, 3H), 2.06 (m, 1H), 1.44 (s, 9H), 1.1 (m, 2H), 0.83 (m, 2H)

MS [M+H]⁺=425.16

### Compound 549

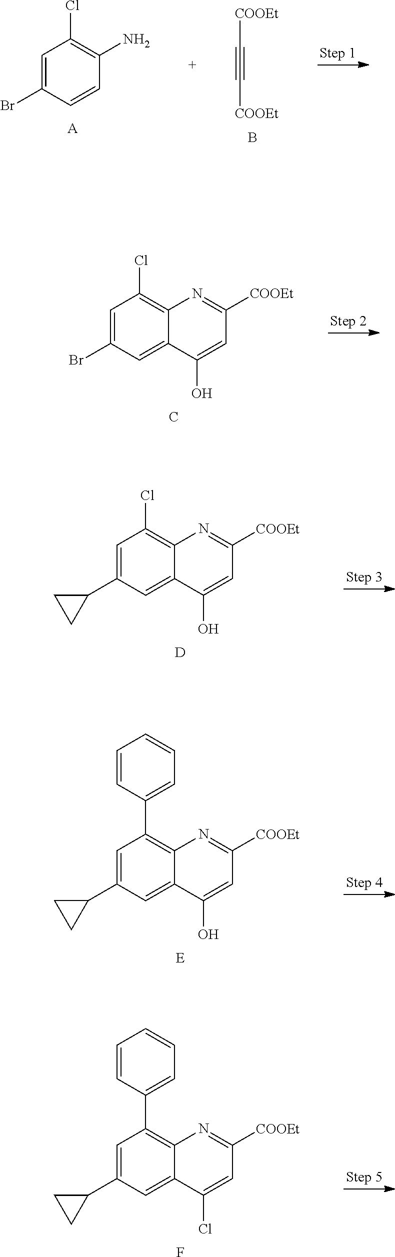

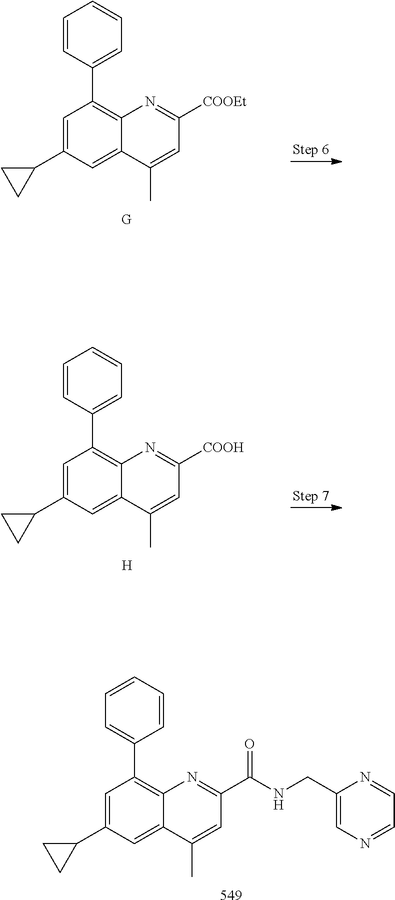

### Step 1

Compound C (6.78 g, 61%) was prepared from compound A and compound B in a manner similar to that described previously. MS [M+H]⁺=329.9

### Step 2 and 3

Compound C (1.122 g, 3.39 mmol), Pd(dppf)Cl₂—CH₂Cl₂, (282 mg, 0.345 mmol), K₂CO₃ (943 mg, 6.82 mmol), and cyclopropylboronic acid hydrate (398 mg, 3.83 mmol) was degassed and dioxane (15 mL) was added. The resulting mixture was refluxed for 4 h. The mixture was dissolved in ethyl acetate and water and the two layers were separated. After the aqueous fraction was extracted with ethyl acetate (×1), the organic fractions were washed with water (×1), combined, dried (Na₂SO₄), and concentrated. The residue was purified by combiflash using hexanes-ethyl acetate to obtain a mixture (1.444 g) of impure compound D. MS [M+H]⁺=292.3

A mixture of the impure compound D, Pd(dppf)Cl₂—CH₂Cl₂, (278 mg, 0.340 mmol), K₂CO₃ (1.892 g, 13.69 mmol), and phenylboronic acid (1.262 g, 10.35 mmol) was degassed and dioxane (20 mL) was added before refluxing for 11 h. To the mixture was added additional phenylboronic acid (350 mg, 2.87 mmol) and the resulting mixture was refluxed for 2 h. After additional Pd(dppf)Cl₂—CH₂Cl₂, (277 mg, 0.339 mmol) was added, the mixture was refluxed for 2 h and the mixture was diluted with ethyl acetate and water before filtration through celite pad. After the two layers of the filtrate were separated and the aqueous fraction was extracted with ethyl acetate (×1), the organic fractions were washed with water (×1), combined, dried (Na₂SO₄), and concentrated. The residue was purified by combiflash using hexanes-ethyl acetate to obtain compound E (499 mg) with some impurities. MS [M+H]⁺=334.2

### Step 4

Compound F (351 mg) was prepared from compound E (499 mg) in a manner similar to that described previously. MS [M+H]⁺=352.2

### Step 5

Compound G (325 mg, 98%) was prepared from compound 27 (351 mg) in a manner similar to that described previously. MS [M+H]⁺=332.3

### Step 6

Compound H (294 mg, 99%) was prepared from compound G (325 mg) in a manner similar to that described previously. MS [M+H]⁺=304.1

### Step 7

Compound 549 (64 mg, 96%) was prepared from compound H (51 mg) in a manner similar to that described previously.

¹H-NMR (400 MHz, CD₃OD) δ 8.78 (br t, J=5.2 Hz, 1H), 8.63 (s, 1H), 8.51 (m, 2H), 8.13 (s, 1H), 7.71 (dd, J=13.6 and 2.0 Hz, 2H), 7.68 (s, 1H), 7.40-7.50 (m, 4H), 4.79 (d, J=5.6 Hz, 2H), 2.79 (s, 3H), 2.17 (m, 1H), 1.14 (m, 2H), 0.90 (m, 2H); MS [M+H]⁺=395.3

### Biological Examples

### Assay Protocol

The anti-HCV activity of the compounds of this invention was tested in a human hepatoma Huh-7 cell line harboring a HCV replicon. The assay comprised the following steps:

### Step 1: Compound Preparation and Serial Dilution.

Serial dilution was performed in 100% DMSO in a 384-well plate. A solution containing a compound at 225-fold concentration of the starting final serial dilution concentration was prepared in 100% DMSO and 15 μL added to the pre-specified wells in column 3 or 13 of a polypropylene 384-well plate. The rest of the 384-well plate was filled with 10 μL 100% DMSO except for columns 23 and 24, where 10 μL of 500 μM a HCV protease inhibitor (ITMN-191) in 100% DMSO was added. The HCV protease inhibitor was used a control of 100% inhibition of HCV replication. The plate was then placed on a Biomek FX Workstation to start the serial dilution. The serial dilution was performed for ten cycles of 3-fold dilution from column 3 to 12 or from column 13 to 22.

### Step 2: Cell Culture Plate Preparation and Compound Addition

To each well of a black polypropylene 384-well plate, 90 μL of cell media containing 1600 suspended Huh-7 HCV replicon cells was added with a Biotek uFlow Workstation. A volume of 0.4 μL of the compound solution was transferred from the serial dilution plate to the cell culture plate on a Biomek FX Workstation. The DMSO concentration in the final assay condition was 0.44%.

The plates were incubated for 3 days at 37° C. with 5% CO2 and 85% humidity.

### Step 3: Detection of Cytotoxicity and Inhibition of Viral Replication

a) Assessment of cytotoxicity: The media in the 384-well cell culture plate was aspirated with a Biotek EL405 plate-washer. A volume of 50 μL of a solution containing 400 nM Calcein AM in 100% PBS was added to each well of the plate with a Biotek uFlow Workstation. The plate was incubated for 30 minutes at room temperature before the fluorescence signal (emission 490 nm, exitation 520 nm) was measured with a Perkin Elmer Envision Plate Reader.

b) Assessment of inhibition of viral replication: The calcein-PBS solution in the 384-well cell culture plate was aspirated with a Biotek EL405 plate-washer. A volume of 20 μL of Dual-Glo luciferase buffer (Promega, Dual-Glo Luciferase Assay Reagent, cat. #E298B) was added to each well of the plate with a Biotek uFlow Workstation. The plate was incubated for 10 minutes at room temperature. A volume of 20 μL of a solution containing 1:100 mixture of Dual-Glo Stop & Glo substrate(Promega, Dual-Glo Luciferase Assay Reagent, cat. #E313B) and Dual-Glo Stop & Glo buffer (Promega, Dual-Glo Luciferase Assay Reagent, cat. #E314B) was then added to each well of the plate with a Biotek uFlow Workstation. The plate was incubated at room temperature for 10 minutes before the luminescence signal was measured with a Perkin Elmer Envision Plate Reader.

### Step 4: Calculation

The percent cytotoxicity was determined by calcein AM conversion to fluorescent product. The average fluorescent signal from the DMSO control wells were defined as 100% nontoxic. The individual fluorescent signal from testing compound treated well was divided by the average signal from DMSO control wells and then multiplied by 100% to get the percent viability. The percent anti-HCV replication activity was determined by the luminescence signal from the testing well compared to DMSO controls wells. The background signal was determined by the average luminescence signal from the HCV protease inhibitor treated wells and was subtracted from the signal from the testing wells as well as the DMSO control wells. Following 3-fold serial dilutions, the EC₅₀ and CC₅₀ values were calculated by fitting % inhibition at each concentration to the following equation:

% inhibition=100%/[(EC₅₀/[I])^{b}+1]

Where b is Hill's coefficient. See, for reference, Hill, A. V., The Possible Effects of the Aggregation of the Molecules of Haemoglobin on its Dissociation Curves, J. Physiol. 40: iv-vii. (1910).

% inhibition values at a specific concentration, for example 2 μM, can also be derived from the formula above.

When tested, certain compounds of this invention were found to inhibit viral replication as listed in Table 1:

TABLE 1

| | | % inhibition @ 10 μM | |
|---|---|---|---|
| | | HCV | HCV |
| Example | | Replicon | Replicon |
| Number | Structure | IB | IA |
| 5 | 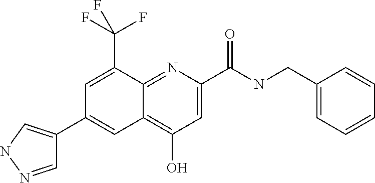 | 99.22 | 99.24 |
| 7 | 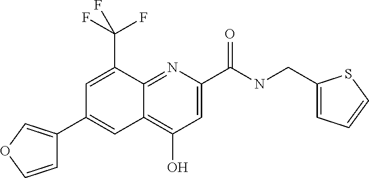 | 99.77 | 99.18 |
| 8 | 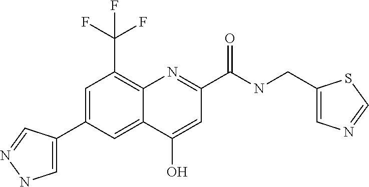 | 94.04 | 98.80 |
| 9 | 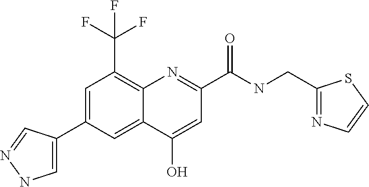 | 98.51 | 98.09 |
| 10 | 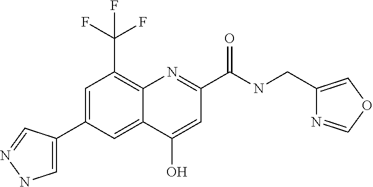 | 74.27 | 80.66 |
| 11 | 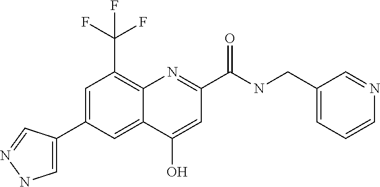 | 66.09 | 63.19 |
| 12 | 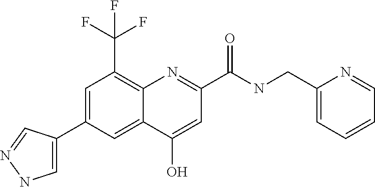 | 99.24 | 98.21 |
| 13 | 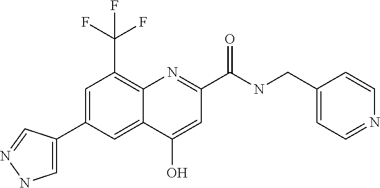 | 20.73 | 43.40 |
| 14 | 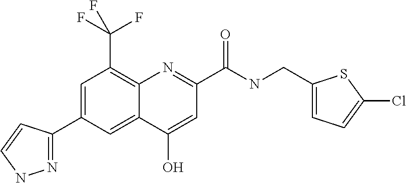 | 68.57 | 84.19 |
| 15 | 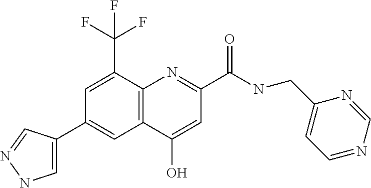 | 47.34 | 59.32 |
| 16 | 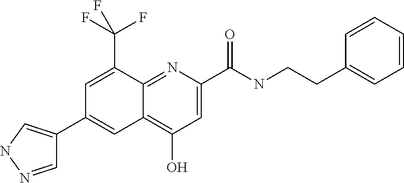 | 96.65 | 92.97 |
| 17 | 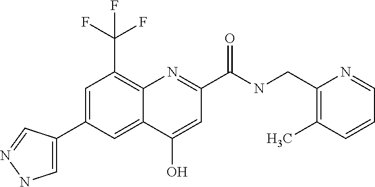 | 96.44 | 98.34 |
| 18 | 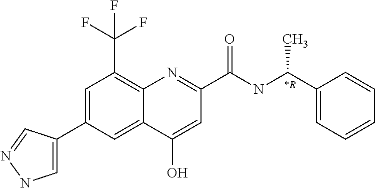 | 55.80 | 55.27 |
| 19 | 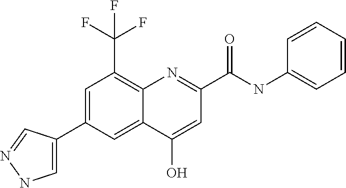 | 71.12 | 98.45 |
| 20 | 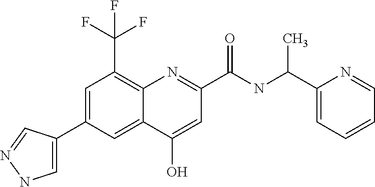 | 89.04 | 86.07 |
| 23 | 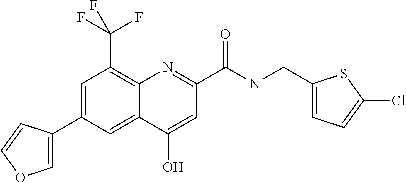 | 99.39 | 99.88 |
| 25 | 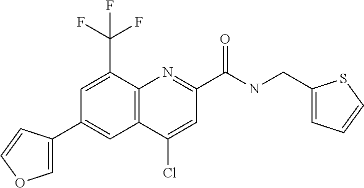 | 99.66 | 98.70 |
| 27 | 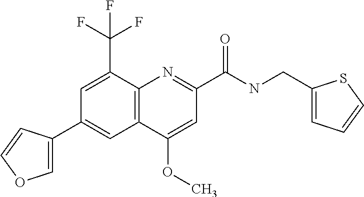 | 80.34 | 65.47 |
| 28 | 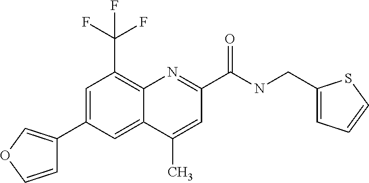 | 99.98 | 99.82 |
| 29 | 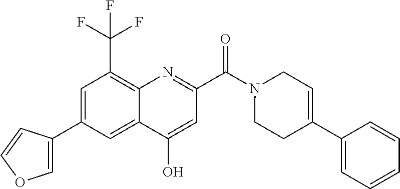 | 37.69 | 87.42 |
| 30 | 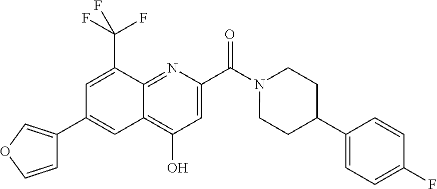 | 74.88 | 95.33 |
| 31 | 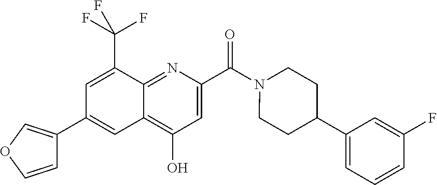 | 58.12 | 98.10 |
| 34 | 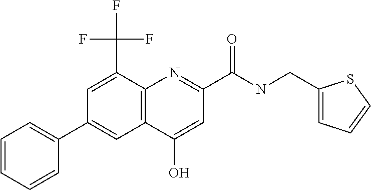 | 98.34 | 97.85 |
| 35 | 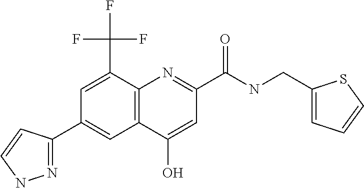 | 94.52 | 94.23 |
| 36 | 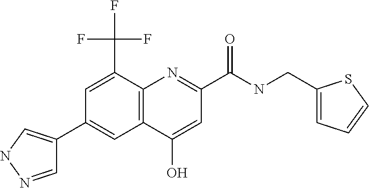 | 99.44 | 99.28 |
| 39 | 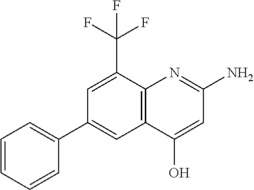 | 64.96 | 92.51 |
| 40 | 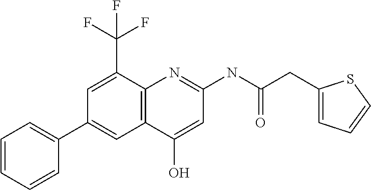 | 97.58 | 99.74 |
| 47 | 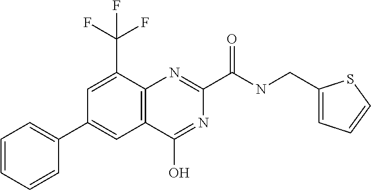 | 99.84 | 99.71 |
| 48 | 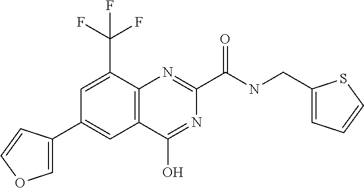 | 99.93 | 99.37 |
| 49 | 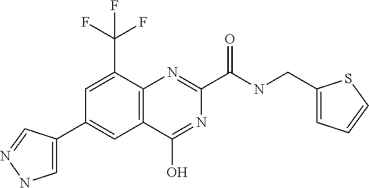 | 99.02 | 99.68 |
| 51 | 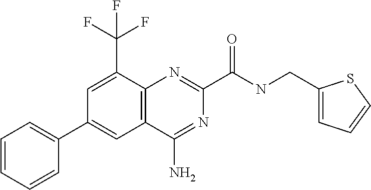 | 99.96 | 99.99 |
| 52 | 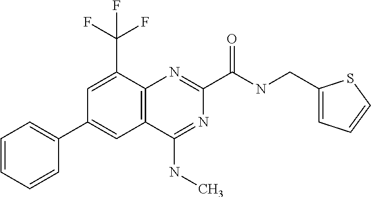 | 99.96 | 99.63 |
| 53 | 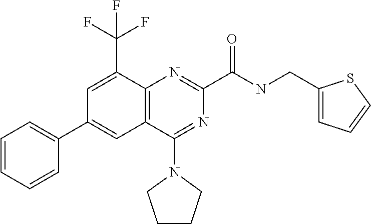 | 86.19 | 91.40 |
| 54 | 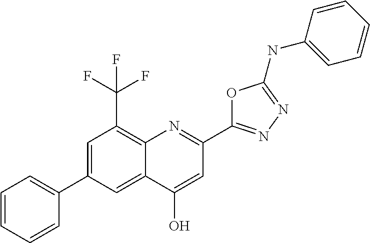 | 98.73 | 97.92 |
| 59 | 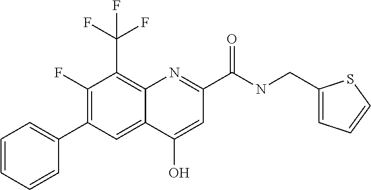 | 97.21 | 92.66 |
| 64 | 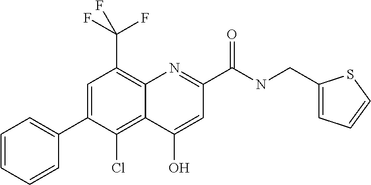 | 88.17 | 79.24 |
| 65 | 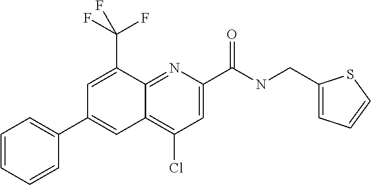 | 99.47 | 97.45 |
| 66 | 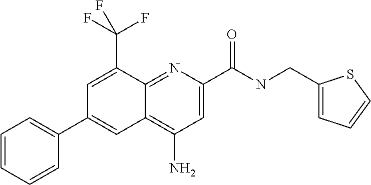 | 99.93 | 99.87 |
| 67 | 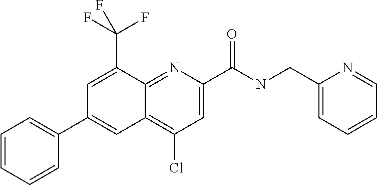 | 97.75 | 96.87 |
| 68 | 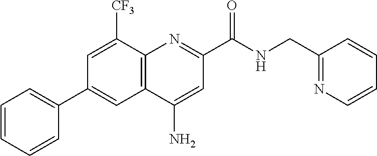 | 99.66 | 99.89 |
| 70 | 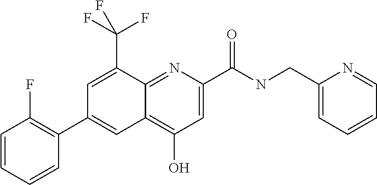 | 82.02 | 69.12 |

| Exam- | | Inhibition @ 10 |
|---|---|---|
| ple # | MOLSTRUCTURE | uM in 1b-Rluc |
| 174 | 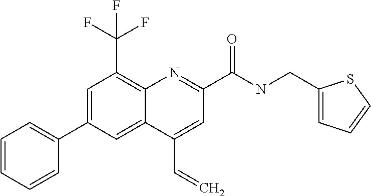 | 63.62 |
| 222 | 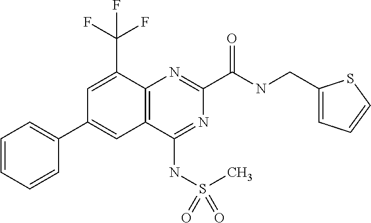 | 95.10 |
| 149 | 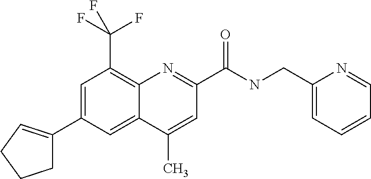 | 99.38 |
| 150 | 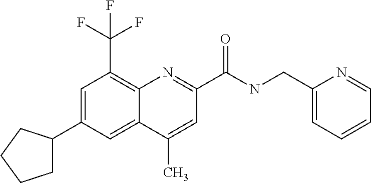 | 99.05 |
| 151 | 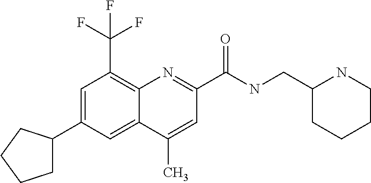 | 99.58 |
| 171 | 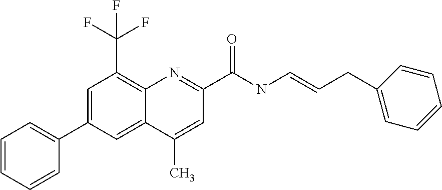 | 92.47 |
| 170 | 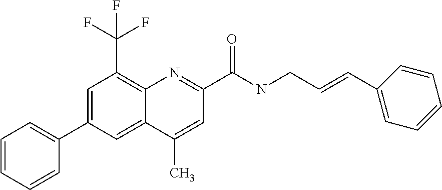 | 66.44 |
| 169 | 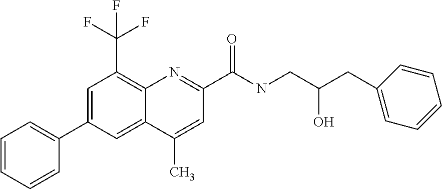 | 93.01 |
| 173 | 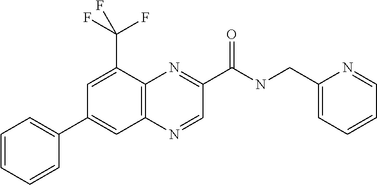 | 86.26 |
| 172 | 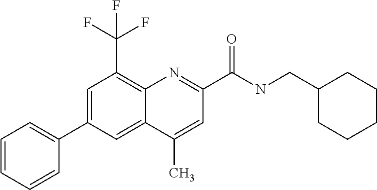 | 95.20 |
| 168 | 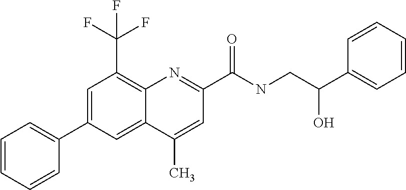 | 86.36 |
| 167 | 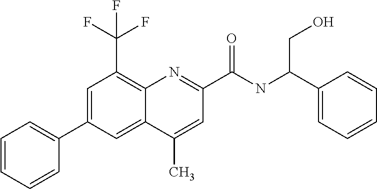 | 87.54 |
| 175 | 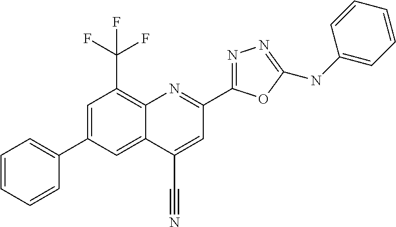 | 89.32 |
| 79 | 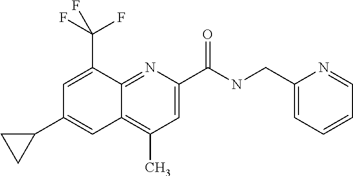 | 99.98 |
| 184 | 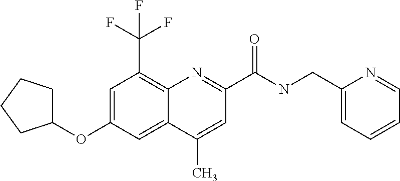 | 95.96 |
| 183 | 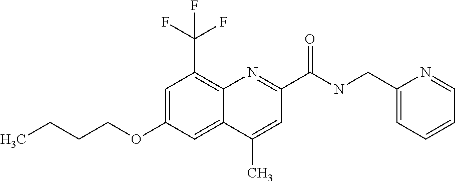 | 88.85 |
| 176 | 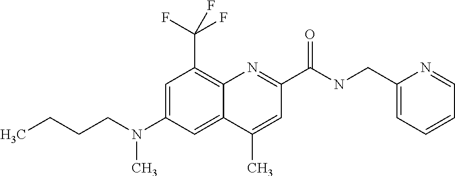 | 80.43 |
| 156 | 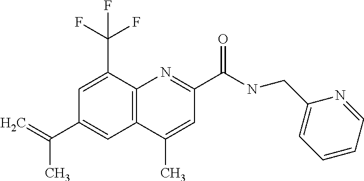 | 99.64 |
| 157 | 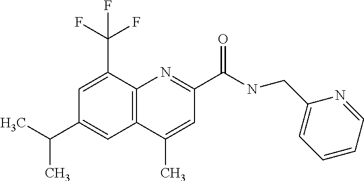 | 98.78 |
| 166 | 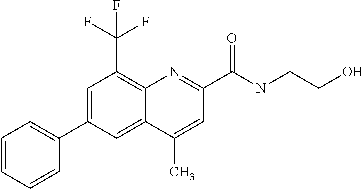 | 93.85 |
| 165 | 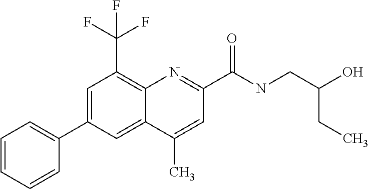 | 96.52 |
| 105 | 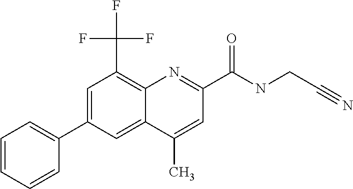 | 82.85 |
| 104 | 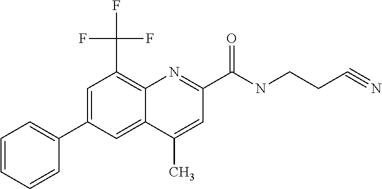 | 70.31 |
| 164 | 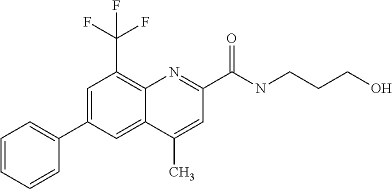 | 94.61 |
| 163 | 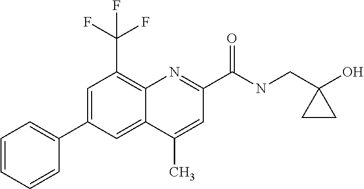 | 98.32 |
| 162 | 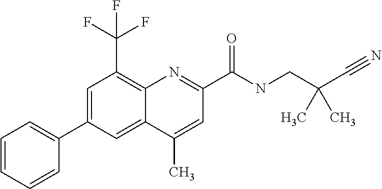 | 80.54 |
| 182 | 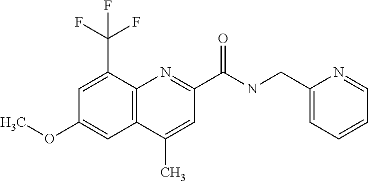 | 99.35 |
| 144 | 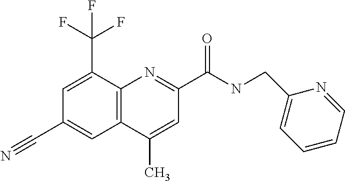 | 57.91 |
| 81 | 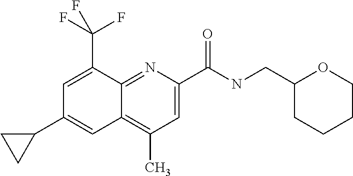 | 99.98 |
| 146 | 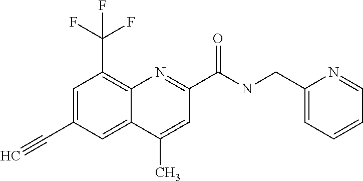 | 99.43 |
| 143 | 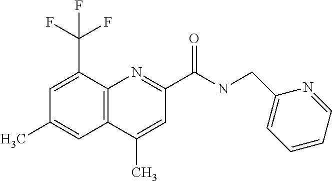 | 93.71 |
| 152 | 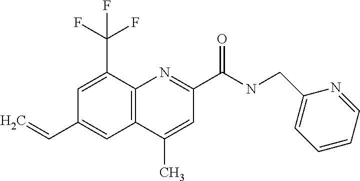 | 99.88 |
| 158 | 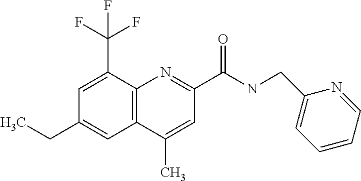 | 98.89 |
| 138 | 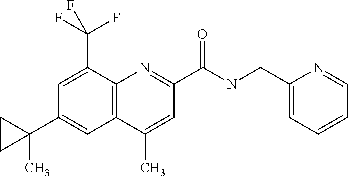 | 98.97 |
| 145 | 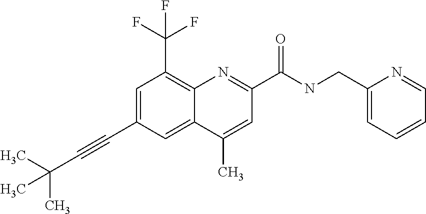 | 72.01 |
| 160 | 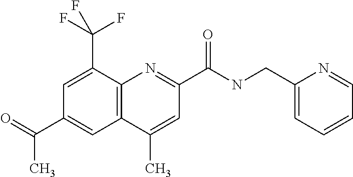 | 93.51 |
| 161 | 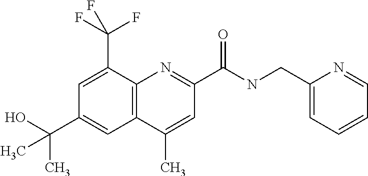 | 76.68 |
| 305 | 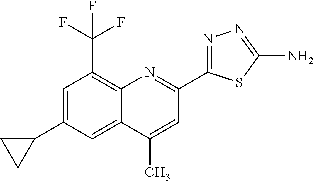 | 96.60 |
| 147 | 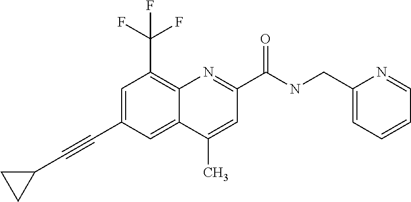 | 94.12 |
| 148 | 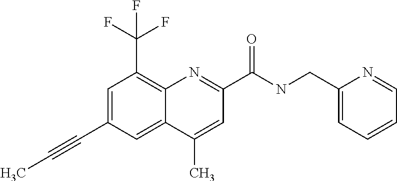 | 99.47 |
| 181 | 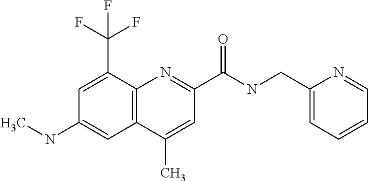 | 99.37 |
| 242 | 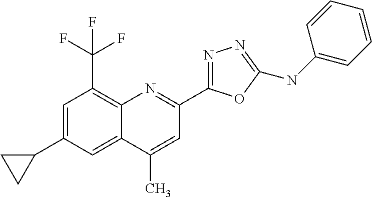 | 99.98 |
| 273 | 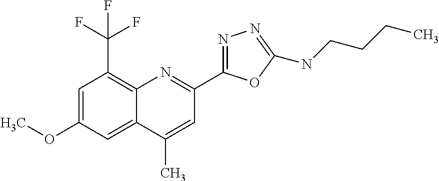 | 100.00 |
| 243 | 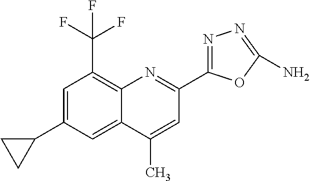 | 88.41 |
| 95 | 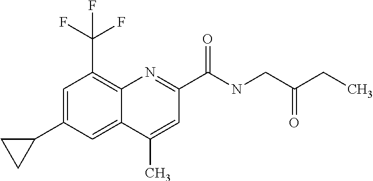 | 99.99 |
| 180 | 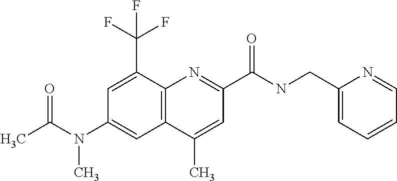 | 65.46 |
| 82 | 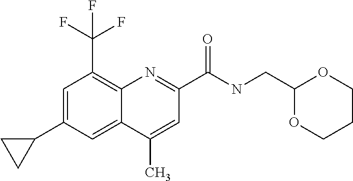 | 99.99 |
| 179 | 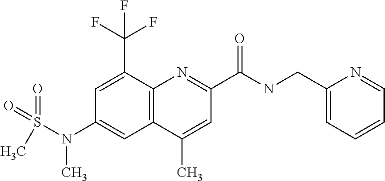 | 98.90 |
| 178 | 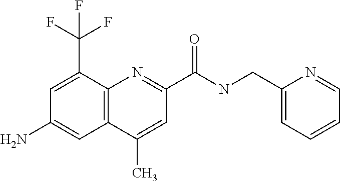 | 86.90 |
| 83 | 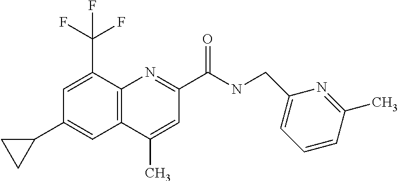 | 99.42 |
| 84 | 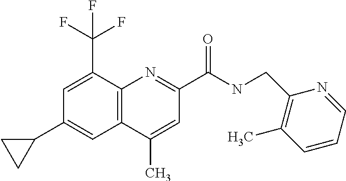 | 99.04 |
| 85 | 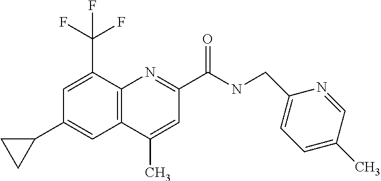 | 100.00 |
| 86 | 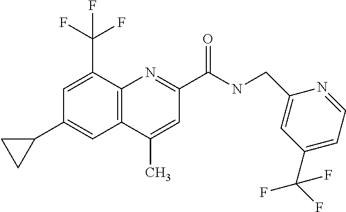 | 100.00 |
| 177 | 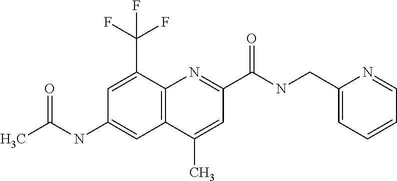 | 94.67 |
| 89 | 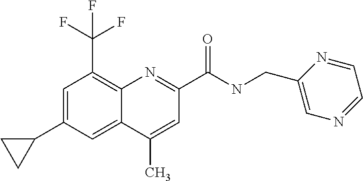 | 99.99 |
| 90 | 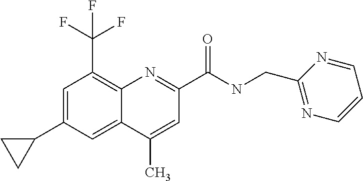 | 99.98 |
| 91 | 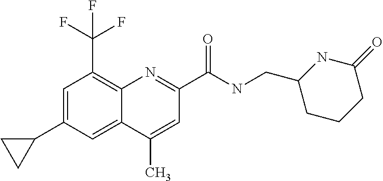 | 92.67 |
| 244 | 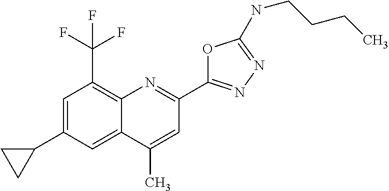 | 99.99 |
| 245 | 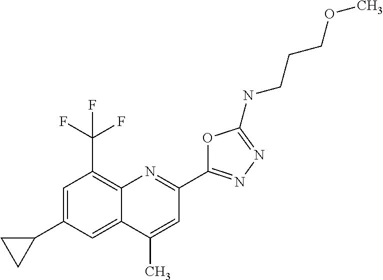 | 99.97 |
| 92 | 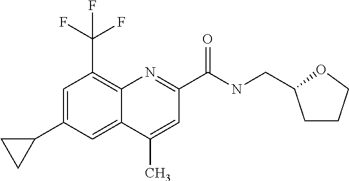 | 99.97 |
| 288 | 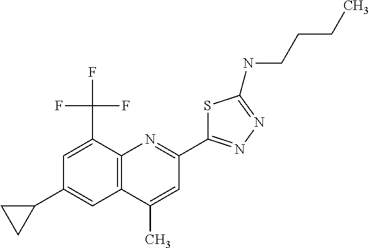 | 99.97 |
| 93 | 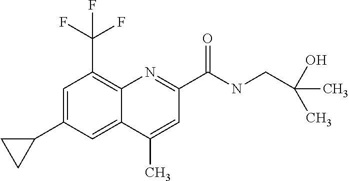 | 98.98 |
| 289 | 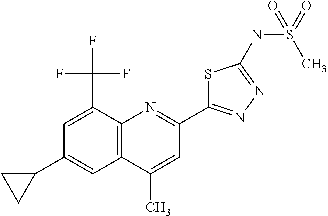 | 70.68 |
| 94 | 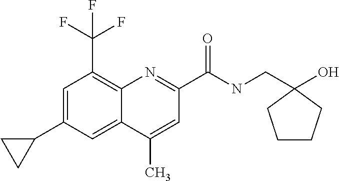 | 99.93 |
| 87 | 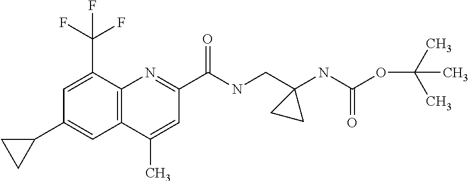 | 84.47 |
| 88 | 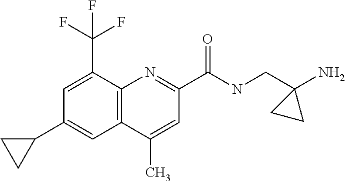 | 95.64 |
| 290 | 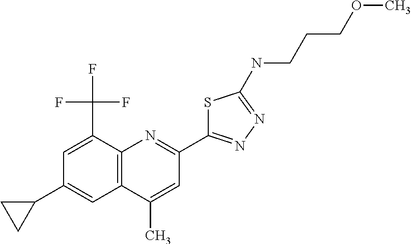 | 99.89 |
| 220 | 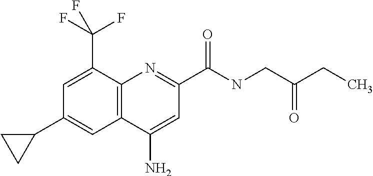 | 100.00 |
| 221 | 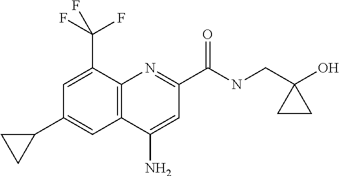 | 98.76 |
| 231 | 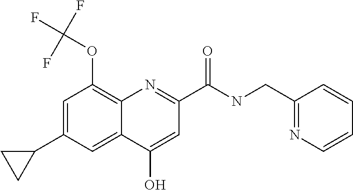 | 98.55 |
| 96 | 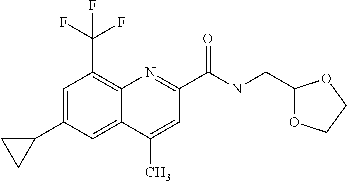 | 99.89 |
| 97 | 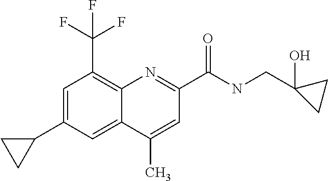 | 98.78 |
| 187 | 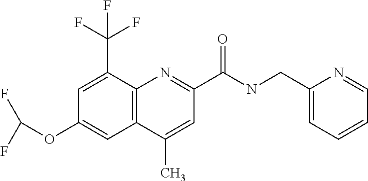 | 99.89 |
| 98 | 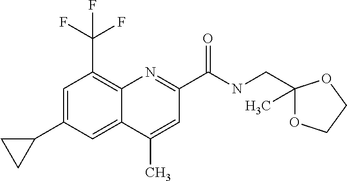 | 98.10 |
| 100 | 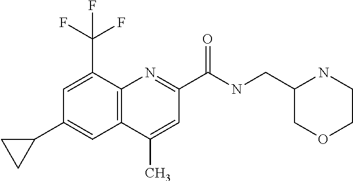 | 99.48 |
| 107 | 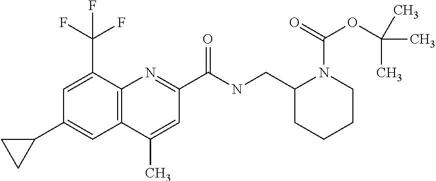 | 94.47 |
| 210 | 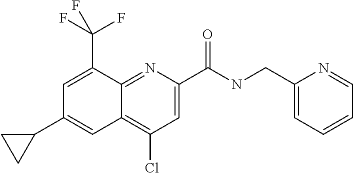 | 99.94 |
| 101 | 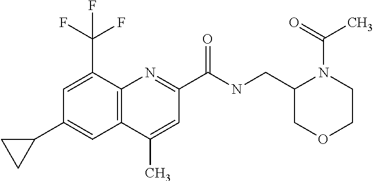 | 73.27 |
| 291 | 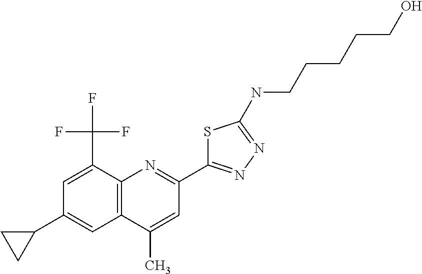 | 99.10 |
| 292 | 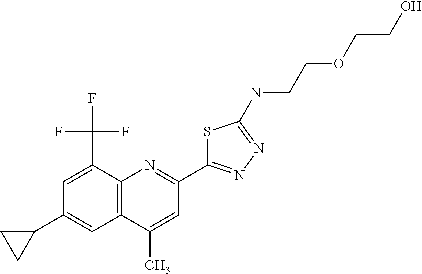 | 97.86 |
| 286 | 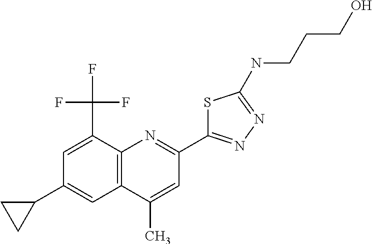 | 99.94 |
| 293 | 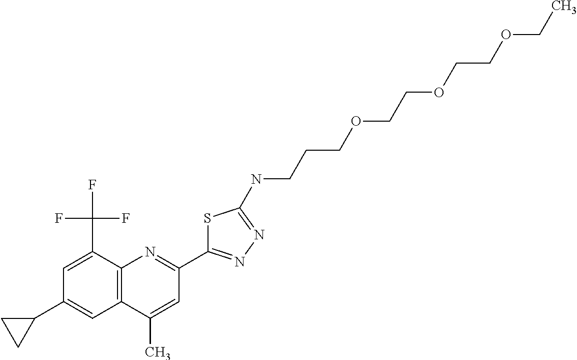 | 96.01 |
| 102 | 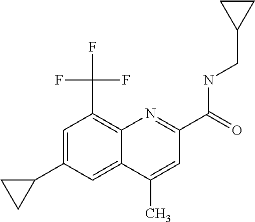 | 99.66 |
| 108 | 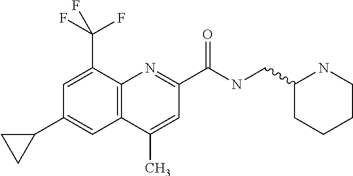 | 96.45 |
| 294 | 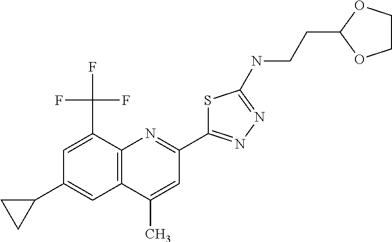 | 99.87 |
| 295 | 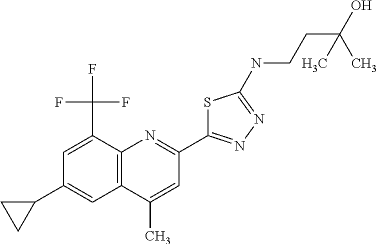 | 98.14 |
| 296 | 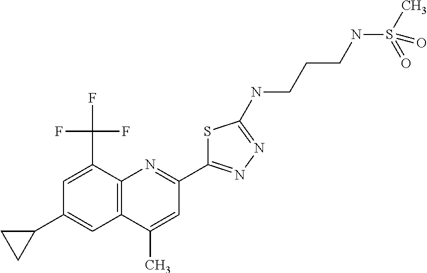 | 51.34 |
| 226 | 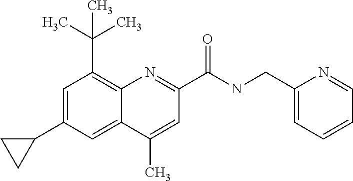 | 99.99 |
| 211 | 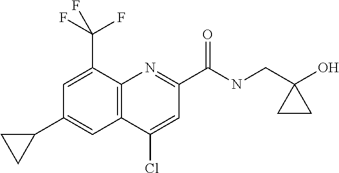 | 94.83 |
| 297 | 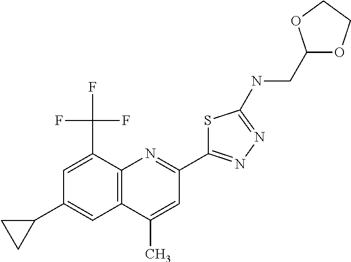 | 99.99 |
| 103 | 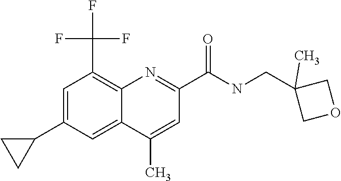 | 96.08 |
| 106 | 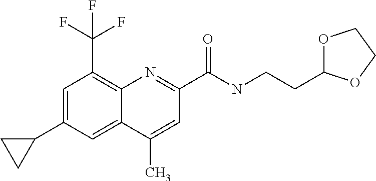 | 99.72 |
| 139 | 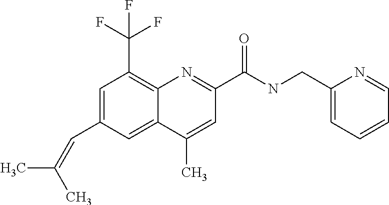 | 99.72 |
| 140 | 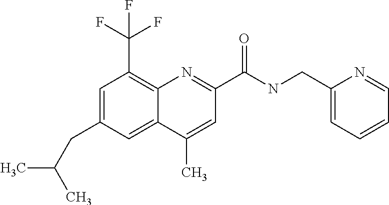 | 99.07 |
| 215 | 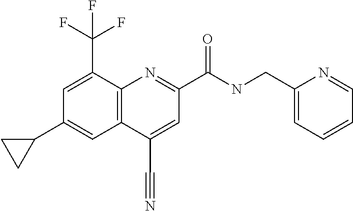 | 99.37 |
| 214 | 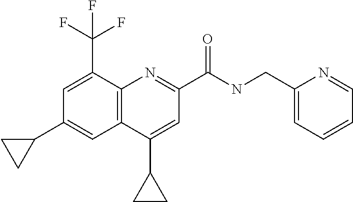 | 92.56 |
| 246 | 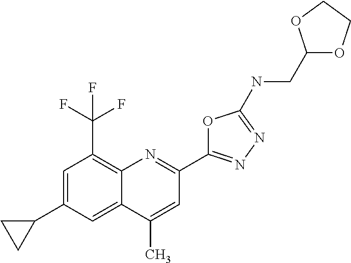 | 100.00 |
| 247 | 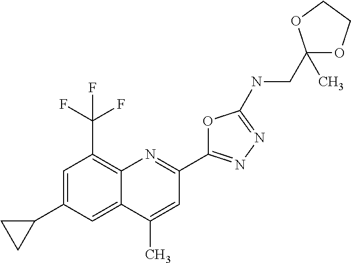 | 100.00 |
| 248 | 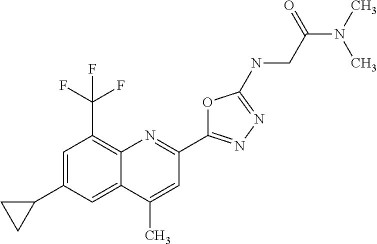 | 99.93 |
| 249 | 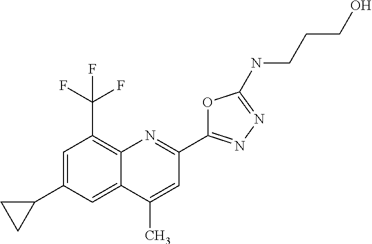 | 99.97 |
| 250 | 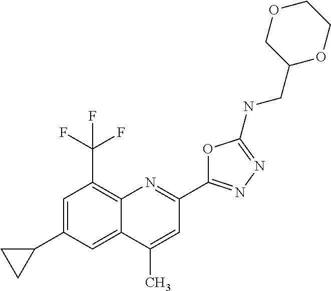 | 99.94 |
| 110 | 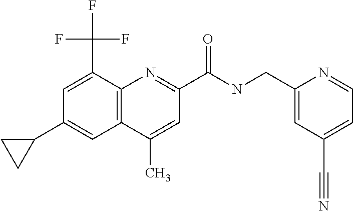 | 99.91 |
| 112 | 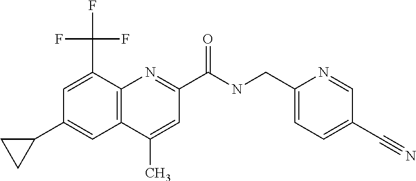 | 99.69 |
| 142 | 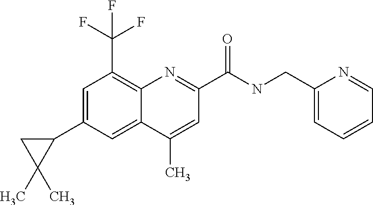 | 99.44 |
| 113 | 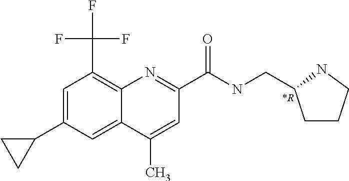 | 96.25 |
| 116 | 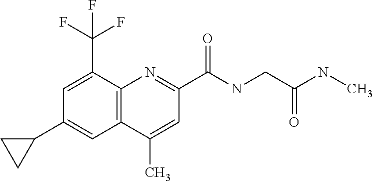 | 97.22 |
| 117 | 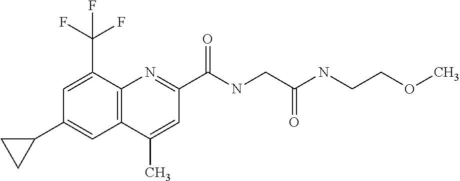 | 94.66 |
| 118 | 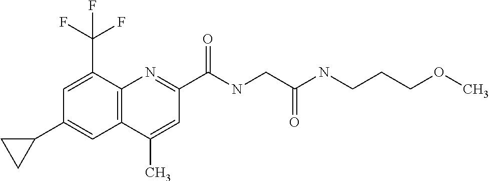 | 84.54 |
| 119 | 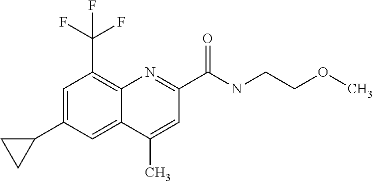 | 99.55 |
| 120 | 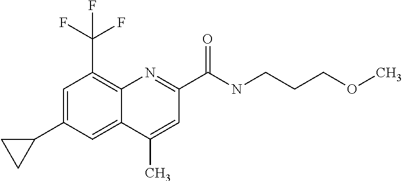 | 99.76 |
| 251 | 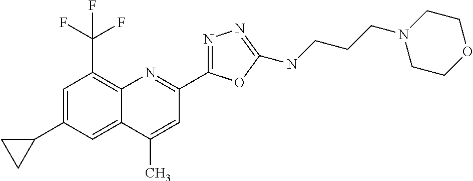 | 80.92 |
| 252 | 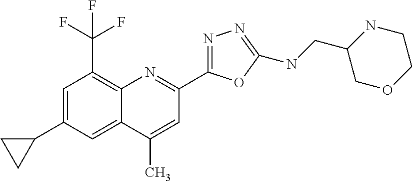 | 92.67 |
| 216 | 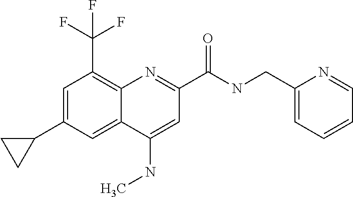 | 98.15 |
| 122 | 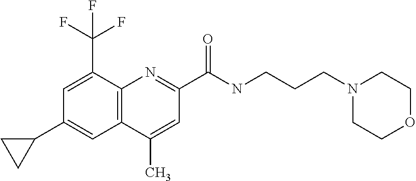 | 97.24 |
| 121 | 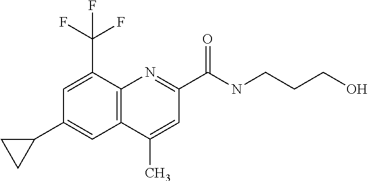 | 99.15 |
| 274 | 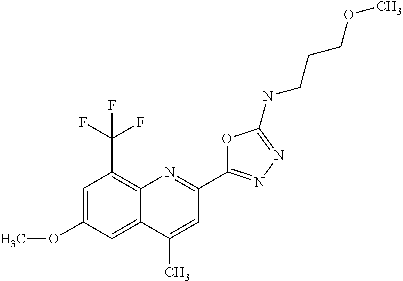 | 99.40 |
| 275 | 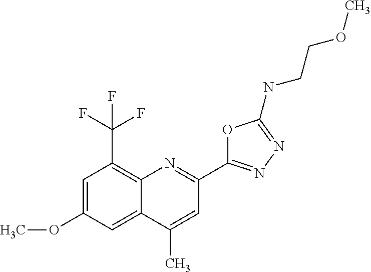 | 99.70 |
| 276 | 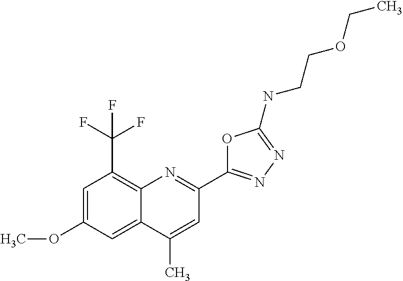 | 97.61 |
| 277 | 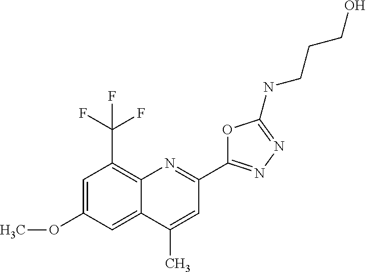 | 98.24 |
| 141 | 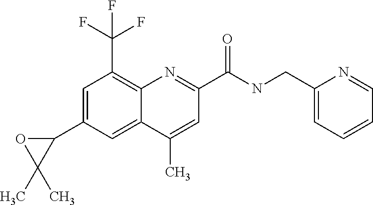 | 94.84 |
| 234 | 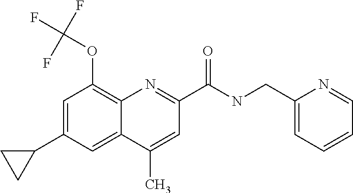 | 99.99 |
| 262 | 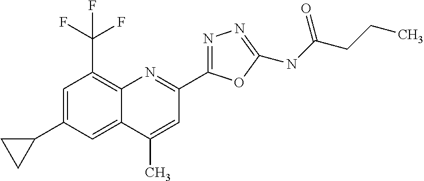 | 99.66 |
| 298 | 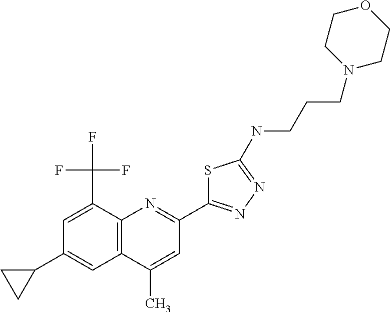 | 88.04 |
| 299 | 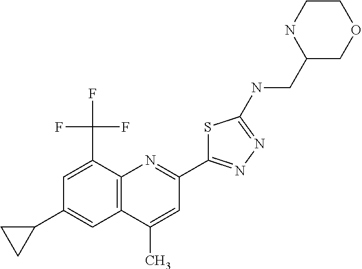 | 97.98 |
| 300 | 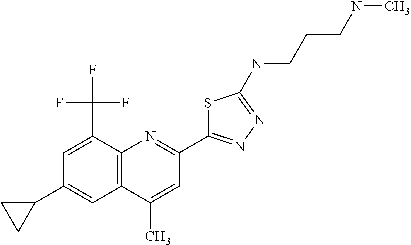 | 97.87 |
| 123 | 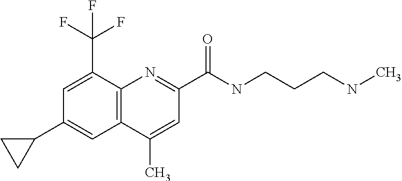 | 96.65 |
| 124 | 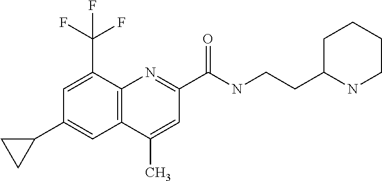 | 99.21 |
| 125 | 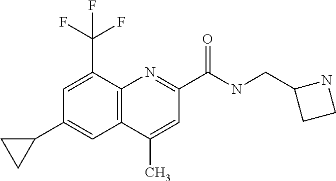 | 95.16 |
| 253 | 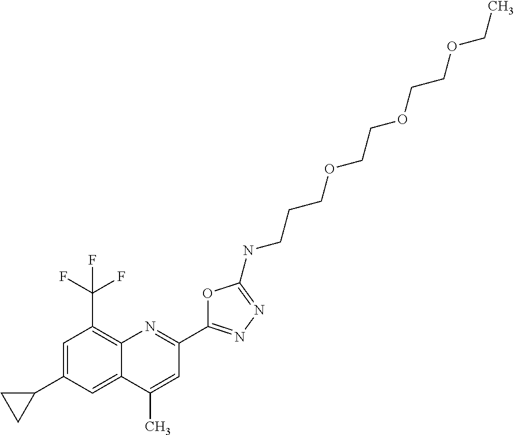 | 80.03 |
| 254 | 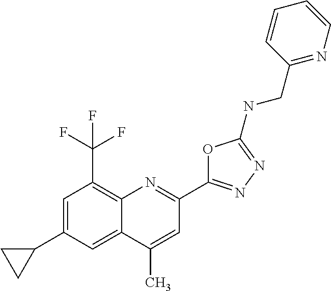 | 99.68 |
| 278 | 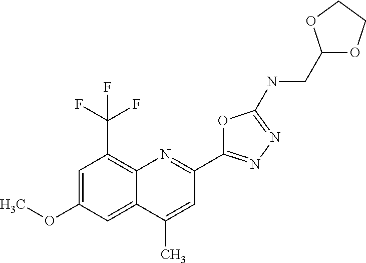 | 99.89 |
| 264 | 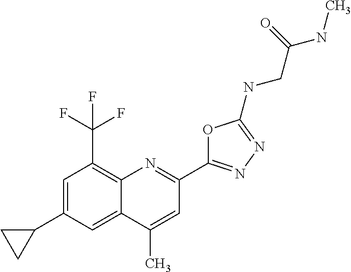 | 99.78 |
| 229 | 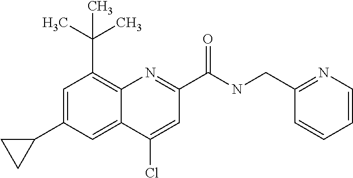 | 99.95 |
| 228 | 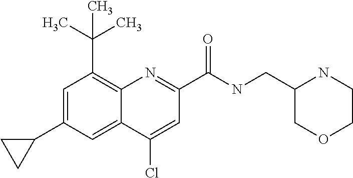 | 94.68 |
| 227 | 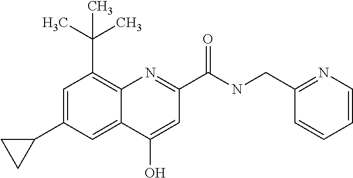 | 99.96 |
| 279 | 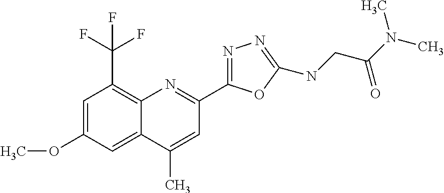 | 98.40 |
| 255 | 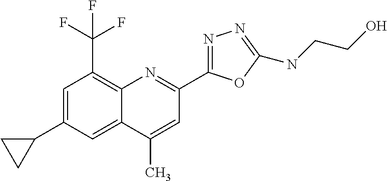 | 99.43 |
| 256 | 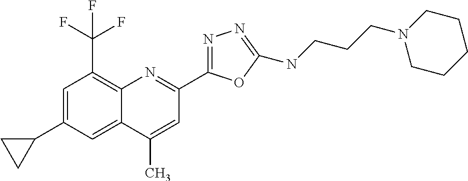 | 99.40 |
| 301 | 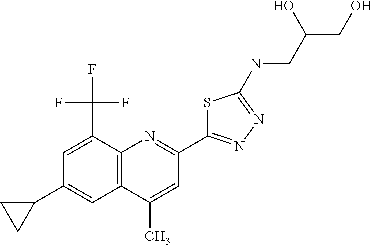 | 99.58 |
| 213 | 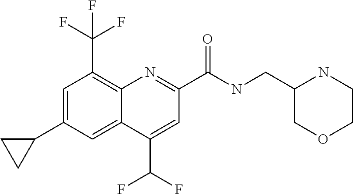 | 88.70 |
| 126 | 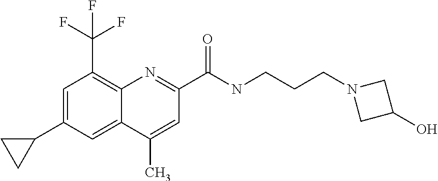 | 90.08 |
| 265 | 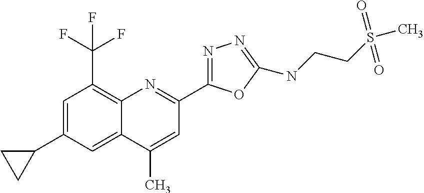 | 98.05 |
| 266 | 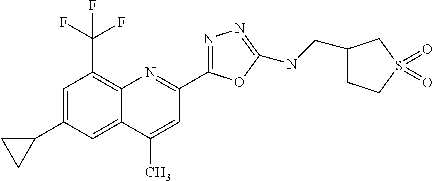 | 90.60 |
| 233 | 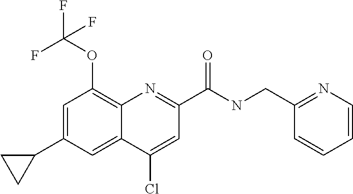 | 99.96 |
| 257 | 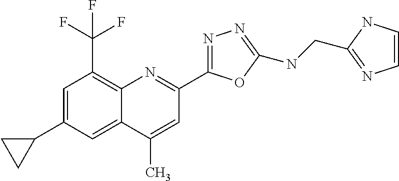 | 99.72 |
| 128 | 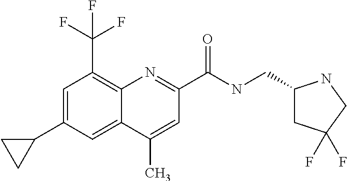 | 99.4 |
| 129 | 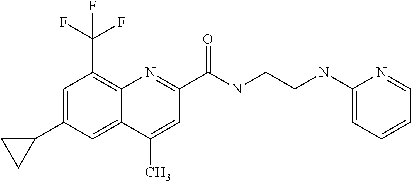 | 94.94 |
| 268 | 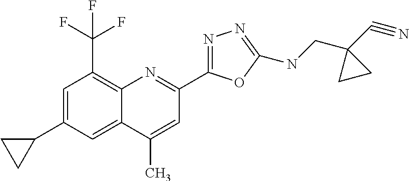 | 99.37 |
| 281 | 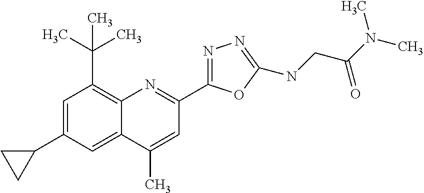 | 99.85 |
| 258 | 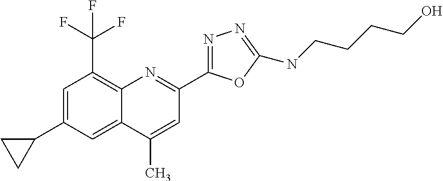 | 99.97 |
| 132 | 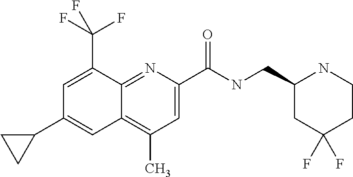 | 99.95 |
| 287 | 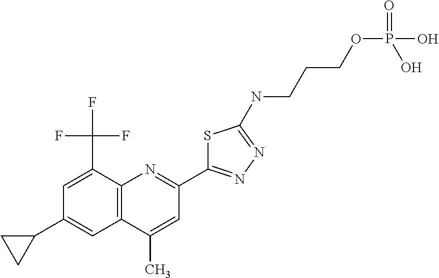 | 99.89 |
| 218 | 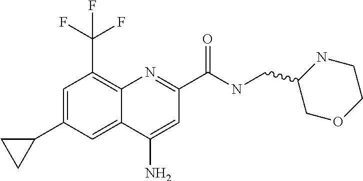 | 99.86 |
| 217 | 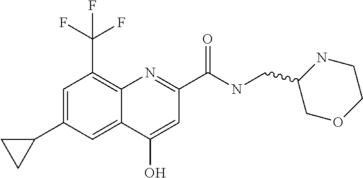 | 77.00 |
| 127 | 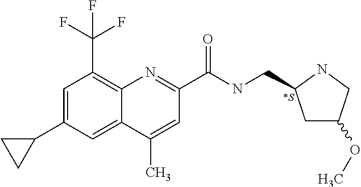 | 99.74 |
| 130 | 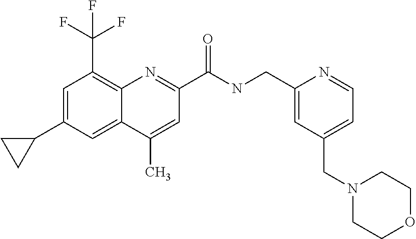 | 84.73 |
| 131 | 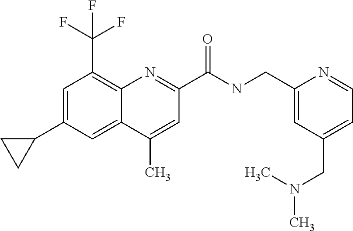 | 85.85 |
| 282 | 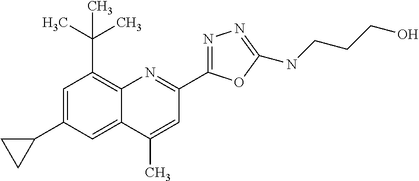 | 99.94 |
| 283 | 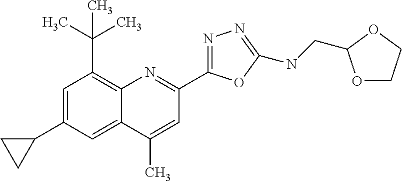 | 100.00 |
| 133 | 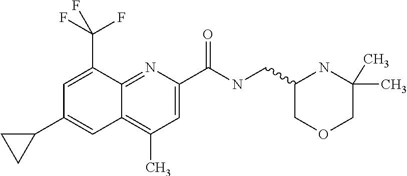 | 92.43 |
| 134 | 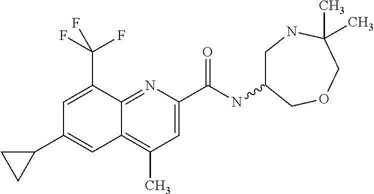 | 93.14 |
| 194 | 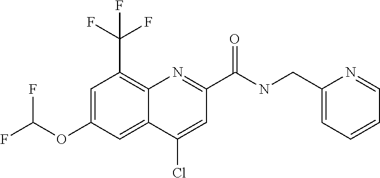 | 99.49 |
| 154 | 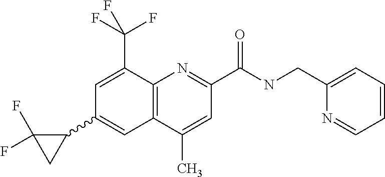 | 99.98 |
| 302 | 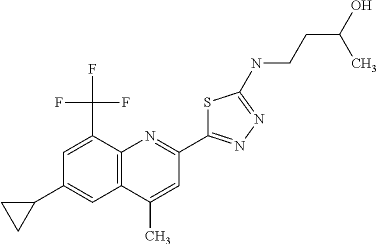 | 99.93 |
| 280 | 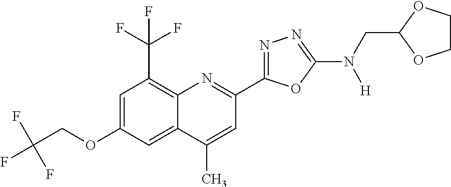 | 99.92 |
| 155 | 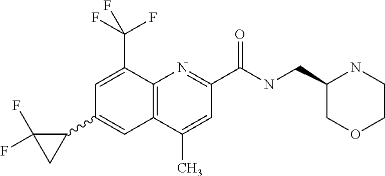 | 99.71 |
| 191 | 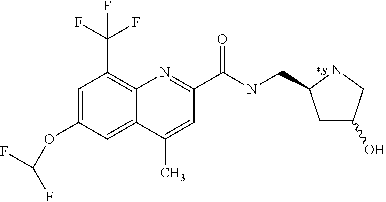 | 50.23 |
| 189 | 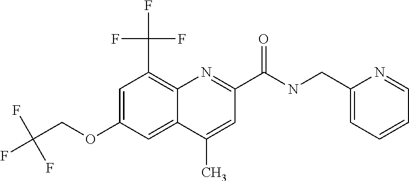 | 99.78 |
| 190 | 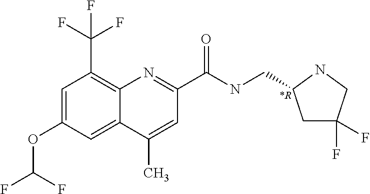 | 94.69 |
| 200 | 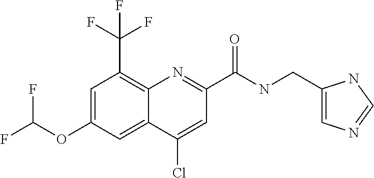 | 90.86 |
| 80 | 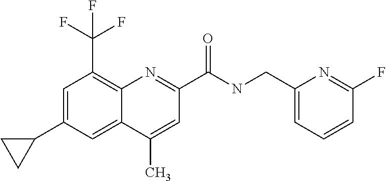 | 99.79 |
| 230 | 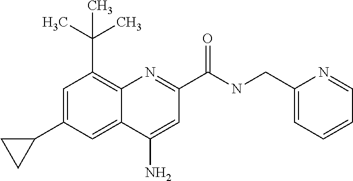 | 99.99 |
| 231 | 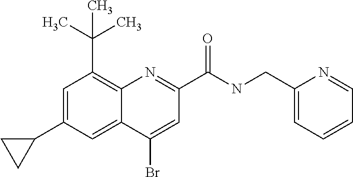 | 99.05 |
| 267 | 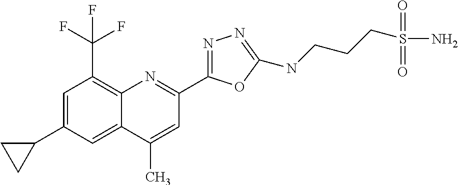 | 97.71 |
| 269 | 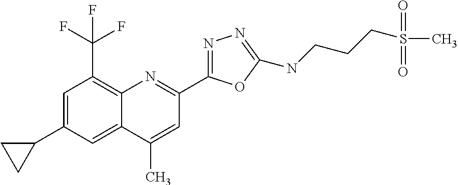 | 87.39 |
| 259 | 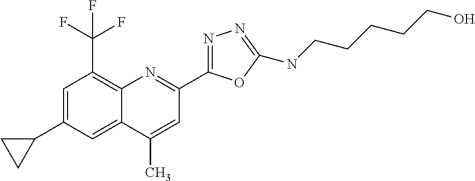 | 98.44 |
| 240 | 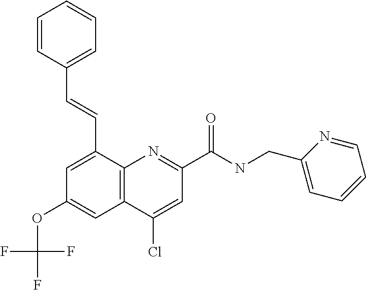 | 42.23 |
| 241 | 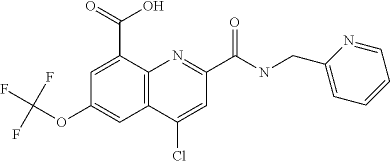 | 7.94 |
| 236 | 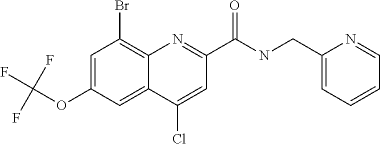 | 86.39 |
| 203 | 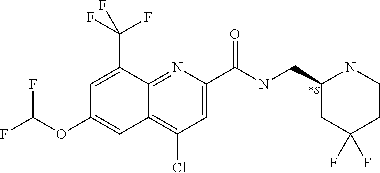 | 98.30 |
| 202 | 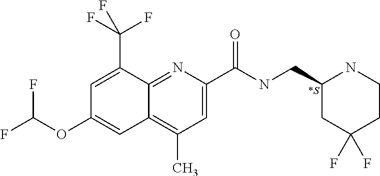 | 99.50 |
| 237 | 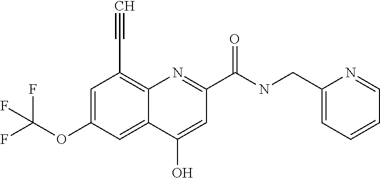 | 21.67 |
| 238 | 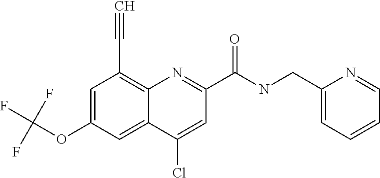 | 9.91 |
| 199 | 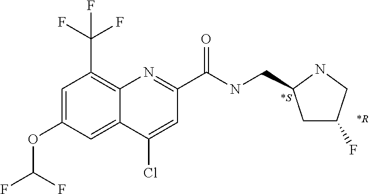 | 98.08 |
| 284 | 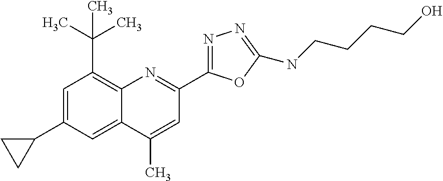 | 99.88 |
| 285 | 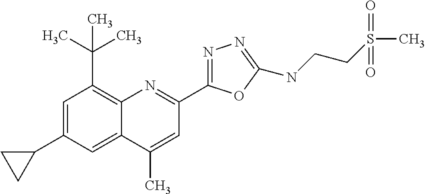 | 99.59 |
| 260 | 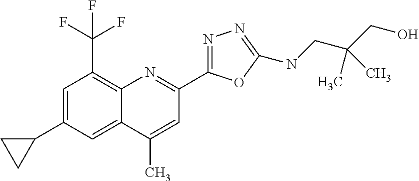 | 96.74 |
| 261 | 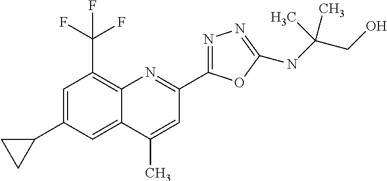 | 99.15 |
| 263 | 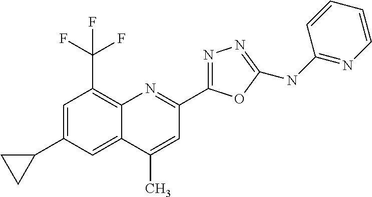 | 98.70 |
| 207 | 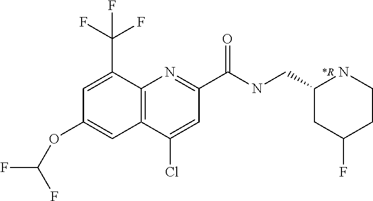 | 86.20 |
| 196 | 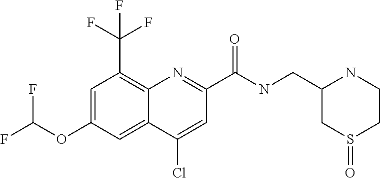 | 54.99 |
| 197 | 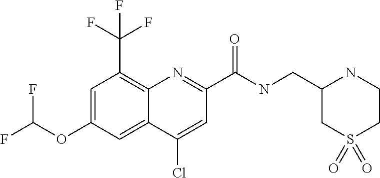 | 92.75 |
| 303 | 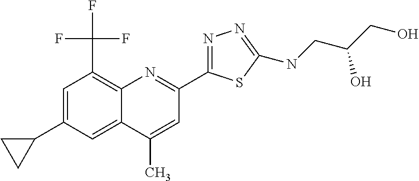 | 98.15 |
| 235 | 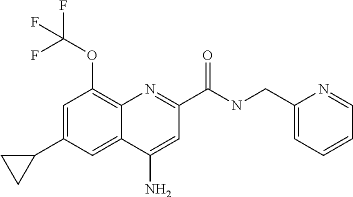 | 99.99 |
| 198 | 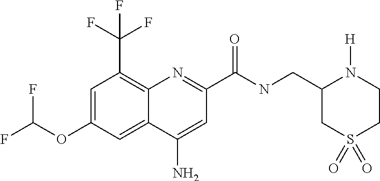 | 98.39 |
| 304 | 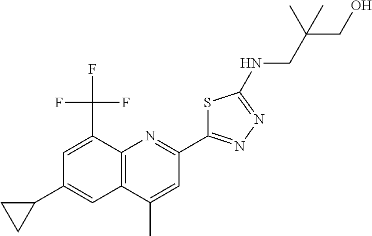 | 96.21 |
| 239 | 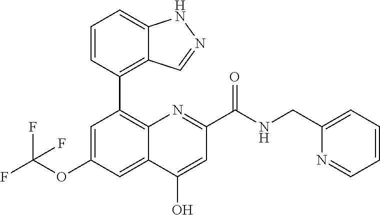 | 49.33 |
| 271 | 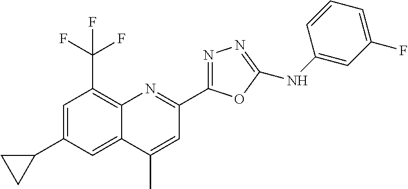 | 99.99 |
| 270 | 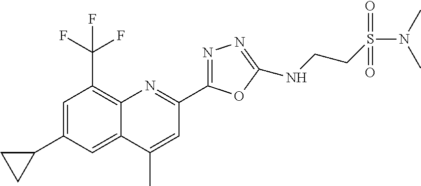 | 84.74 |
| 272 | 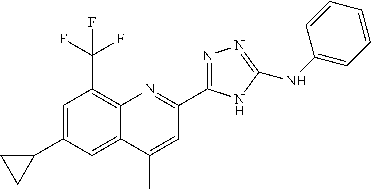 | 93.56 |
| 204 | 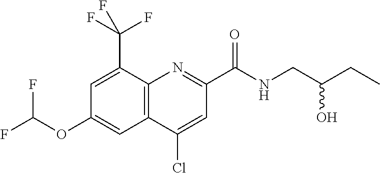 | 95.55 |
| 205 | 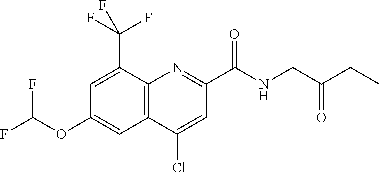 | 99.23 |
| 206 | 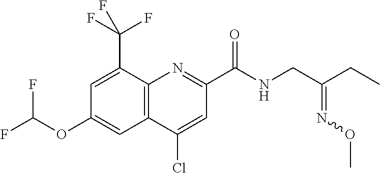 | 63.97 |
| 195 | 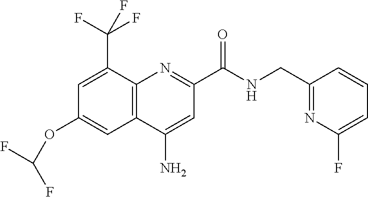 | 99.71 |

The anti-HCV activity of the compounds of this invention was tested in a human hepatoma Huh-7 cell line harboring a HCV replicon as described above were performed on Compound numbers 306-549, and the EC₅₀ values were determined for HCV1B as shown in Table II:

TABLE II
Compounds 306-549

| compound # | EC50_HCV1B_(nM) |
|---|---|
| 306 | 1407.70 |
| 307 | 14.14 |
| 308 | 66.00 |
| 309 | 7089.70 |
| 310 | 1572.70 |
| 311 | 1135.00 |
| 312 | 2117.10 |
| 313 | 10.17 |
| 314 | 26.07 |
| 315 | 83.89 |
| 316 | 61.83 |
| 317 | 9787.10 |
| 318 | 44444.00 |
| 319 | 0.81 |
| 320 | 1.70 |
| 321 | 2.91 |
| 322 | 5.38 |
| 323 | 7.00 |
| 324 | 3.63 |
| 326 | 13.79 |
| 327 | 16.49 |
| 328 | 15.93 |
| 329 | 9.80 |
| 330 | 19.79 |
| 331 | 242.79 |
| 332 | 3.07 |
| 333 | 6.32 |
| 334 | 1.71 |
| 335 | 3.02 |
| 336 | 4.45 |
| 337 | 69.57 |
| 338 | 4705.70 |
| 339 | 1226.80 |
| 340 | 3905.00 |
| 341 | 3531.30 |
| 342 | 2.11 |
| 343 | 2.18 |
| 344 | 11.01 |
| 345 | 559.82 |
| 346 | 156.65 |
| 347 | 7.54 |
| 348 | 5.27 |
| 349 | 26.89 |
| 350 | 1.90 |
| 351 | 24.10 |
| 352 | 218.73 |
| 352 | 12.83 |
| 354 | 15.31 |
| 355 | 3.24 |
| 356 | 10.59 |
| 357 | 356.21 |
| 358 | 1.69 |
| 359 | 1.21 |
| 360 | 44.11 |
| 361 | 7.36 |
| 362 | 1.72 |
| 363 | 2.33 |
| 364 | 2.54 |
| 364 | 2.32 |
| 365 | 4.87 |
| 366 | 2.01 |
| 367 | 2.15 |
| 368 | 17.45 |
| 369 | 29.78 |
| 370 | 2.18 |
| 371 | 1.70 |
| 372 | 1.95 |
| 373 | 65.05 |
| 374 | 61.97 |
| 375 | 9.56 |
| 376 | 0.86 |
| 377 | 6.23 |
| 378 | 48.90 |
| 379 | 1101.70 |
| 380 | 12.68 |
| 381 | 2931.20 |
| 382 | 14.24 |
| 383 | 4.85 |
| 384 | 3.66 |
| 385 | 1.73 |
| 386 | 1.14 |
| 387 | 3.30 |
| 388 | 13.21 |
| 389 | 145.28 |
| 390 | 1.75 |
| 391 | 22.25 |
| 392 | 31.34 |
| 393 | 1199.70 |
| 394 | 1126.00 |
| 395 | 34.37 |
| 396 | 1109.10 |
| 397 | 3.17 |
| 398 | 3.69 |
| 399 | 887.71 |
| 400 | 75.51 |
| 401 | 4.16 |
| 402 | 2.86 |
| 403 | 6.52 |
| 404 | 2.18 |
| 405 | 5.25 |
| 406 | 5.83 |
| 407 | 11.48 |
| 408 | 3.37 |
| 409 | 9.43 |
| 410 | 10.27 |
| 410 | 25.34 |
| 411 | 53.96 |
| 411 | 162.33 |
| 412 | 36.19 |
| 413 | 310.87 |
| 414 | 1.74 |
| 415 | 85.03 |
| 416 | 0.91 |
| 417 | 6.11 |
| 418 | 4.58 |
| 419 | 43.86 |
| 420 | 3.10 |
| 421 | 3.24 |
| 422 | 5.55 |
| 423 | 1.35 |
| 424 | 488.11 |
| 425 | 12.45 |
| 426 | 6.27 |
| 427 | 7.44 |
| 428 | 834.40 |
| 429 | 293.54 |
| 430 | 1449.10 |
| 431 | 8.04 |
| 432 | 83.35 |
| 433 | 1.19 |
| 434 | 1.33 |
| 435 | 7.84 |
| 436 | 588.71 |
| 437 | 20.18 |
| 438 | 45.95 |
| 439 | 1157.50 |
| 440 | 10.16 |
| 441 | 12.37 |
| 442 | 14.94 |
| 443 | 18.75 |
| 444 | 5.88 |
| 445 | 256.69 |
| 446 | 523.10 |
| 447 | 37.50 |
| 447b | 52.14 |
| 448 | 1.57 |
| 449 | 11.31 |
| 450 | 3.15 |
| 451 | 2.45 |
| 452 | 19.65 |
| 453 | 14.93 |
| 454 | 2.27 |
| 455 | 3.05 |
| 456 | 3.30 |
| 457 | 4.10 |
| 458 | 0.83 |
| 459 | 4.32 |
| 460 | 388.09 |
| 461 | 1.38 |
| 462 | 1.54 |
| 463 | 3.76 |
| 464 | 3.43 |
| 465 | 4.99 |
| 466 | 0.88 |
| 467 | 0.63 |
| 468 | 0.36 |
| 469 | 115.36 |
| 470 | 0.72 |
| 471 | 0.94 |
| 472 | 0.30 |
| 473 | 270.05 |
| 474 | 1.02 |
| 475 | 8.54 |
| 476 | 1.32 |
| 477 | 2.22 |
| 478 | 1.48 |
| 479 | 1.14 |
| 480 | 1.24 |
| 481 | 2882.60 |
| 482 | 2.96 |
| 483 | 1.72 |
| 484 | 2187.60 |
| 485 | 1045.70 |
| 486 | 1845.80 |
| 487 | 41.55 |
| 488 | 6.82 |
| 489 | 26.71 |
| 490 | 50.05 |
| 491 | 0.57 |
| 492 | 19.29 |
| 493 | 8.74 |
| 494 | 43.06 |
| 495 | 38.41 |
| 496 | 13.65 |
| 497 | 73.70 |
| 498 | 2.39 |
| 499 | 16.77 |
| 500 | 7.57 |
| 501 | 7.23 |
| 502 | 68.63 |
| 503 | 1.71 |
| 504 | 9.86 |
| 505 | 6.25 |
| 506 | 62.53 |
| 507 | 19.06 |
| 508 | 24.31 |
| 509 | 3.61 |
| 510 | 9.80 |
| 511 | 32.31 |
| 512 | 37.29 |
| 513 | 8.11 |
| 514 | 11.17 |
| 515 | 15.95 |
| 516 | 7.15 |
| 517 | 5.76 |
| 518 | 51.28 |
| 519 | 21.11 |
| 520 | 0.44 |
| 521 | |
| 522 | 5.85 |
| 523 | 232.55 |
| 524 | 1.25 |
| 525 | 0.74 |
| 526 | 1.64 |
| 527 | 0.81 |
| 528 | 0.41 |
| 529 | 1.37 |
| 530 | 2.81 |
| 531 | 6.51 |
| 532 | 41.17 |
| 533 | 305.64 |
| 534 | 153.86 |
| 535 | 2.54 |
| 536 | 31.40 |
| 537 | 69.11 |
| 538 | 66.57 |
| 539 | 4.12 |
| 540 | 36.58 |
| 541 | 18.62 |
| 542 | 43.81 |
| 543 | 154.86 |
| 544 | 46.69 |
| 545 | 7.68 |
| 546 | 7.48 |
| 547 | 184.77 |
| 548 | 37.06 |
| 549 | 65.50 |

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of Formula (3), 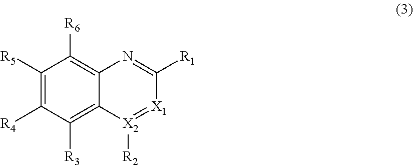
or a pharmaceutically acceptable salt, solvate, tautomer, or prodrug thereof, wherein:
X₁ is CR₆ or N;
X₂ is C or N
R₁ is C(O)NZ₁R₇ or heteroaryl, wherein said heteroaryl is optionally substituted with NZ₁R₇;
R₂ is:
a) not present when X₂ is N, or
b) H, (C₁-C₃)alkyl, (C₂-C₆)alkenyl, (C₂-C₆)alkynyl, (C₁-C₃)alkoxy, hydroxyl, halo, amino, amido, amino(C₁-C₈)alkylamido, heterocyclyl, sulfonyl, aminosulfonyl, amino(C₁-C₈)alkysulfonyl, cyano, or (C₁-C₃)haloalkyl;
provided that (1) when X₁ is N then R₂ is not halogen, and (2) only one of X₁ and X₂ is N;
R₃ is H, halo, (C₁-C₃)alkyl, or (C₁-C₃)haloalkyl;
R₄ is Halo, (C₁-C₆)alkyl, (C₂-C₆)alkenyl, (C₂-C₆)alkynyl, (C₁-C₆)alkoxy, cycloalkyl, heterocyclyl, aryl, heteroaryl, cycloalkoxy, aryloxy, amino, aminosulfonyl, alkylsulfonyl or amido, and wherein any of the preceding substitutents are optionally independently substituted with halo, amino, amido, (C₁-C₆)alkyl, (C₂-C₆)alkenyl, (C₂-C₆)alkynyl, (C₁-C₆)alkoxy, cycloalkyl, heterocyclyl, hydroxyl, and combinations thereof;
R₅ is H or halo;
R₆ is Halo, (C₁-C₈)alkyl, (C₂-C₈)alkenyl, C₂-C₈)alkynyl, (C₁-C₈)haloalkyl, (C₂-C₈)haloalkenyl, (C₂-C₈)haloalkynyl, (C₁-C₈)alkoxy, (C₁-C₈)haloalkoxy, heterocyclyl, heteroaryl, C(O), C(O)OH, hydroxyl, (C₁-C₄)alkylsulfonyl, aminosulfonyl, amino(C₁-C₄)alkylsulfonyl or aryl;
R₇ is H, sulfonyl, (C₁-C₆)alkyl, (C₁-C₆)alkoxy, (C₂-C₆)alkenyl, (C₂-C₆)alkynyl, (C₁-C₆)OH, (C₁-C₆)alkylcycloalkyl, cyano(C₁-C₆)alkyl, (C₁-C₆)alkylcarbonyl, aryl, (C₁-C₆)arylalkyl, (C₁-C₆)alkoxyaryl (C₂-C₆)alkenylaryl (C₁-C₆)alkylheterocycle, or (C₁-C₆)alkylheteroaryl,
wherein any of said (C₁-C₆)alkyl, (C₁-C₆)alkoxy, (C₂-C₆)alkenyl, (C₂-C₆)alkynyl, (C₁-C₆)alcohol, (C₁-C₆)alkylcycloalkyl, (C₁-C₆)alkylnitrile, (C₂-C₆)alkylcarbonyl, aryl, (C₁-C₆)arylalkyl, (C₁-C₆)alkoxyaryl (C₂-C₆)alkenylaryl (C₁-C₆)alkylheterocycle, (C₁-C₆)alkylheteroaryl, are optionally substituted with carbonyl, (C₁-C₄)alkyl, (C₁-C₄)haloalkyl, hydroxyl, C(O)O—R₂₉;
Z₁ is H or C₁-C₆ alkyl;
R₇ and R₈ are independently optionally substituted heterocyclylalkyl, optionally substituted heterocyclylalkylalkyl, optionally substituted aryl, optionally substituted arylalkyl, optionally substituted heteroaryl, optionally substituted heteroarylalkyl, optionally substituted heterocyclylalkynyl, optionally substituted heterocyclylalkenyl, optionally substituted arylalkenyl, optionally substituted arylalkynyl, optionally substituted cycloalkyl, optionally substituted cycloalkylalkyl, optionally substituted alkyl, or H; or,
Z₁ and R₇, together with the nitrogen atom to which they are attached, form a 5 or 6 membered heterocycle, said heterocycle optionally substituted with aryl or heteroaryl; and
R₂₉ is H or (C₁-C₄)alkyl.

2. The compound of claim 1 wherein X₂ is C.

3. The compound of claim 1 wherein X₂ is N, and R₂ is not present.

4. The compound of claim 1 wherein X₁ is CR₆ and X₂ is C.

5. The compound of claim 1 wherein R₃ is H.

6. The compound of claim 5 wherein R₅ is H or F.

7. The compound of claim 1, wherein R₁ is —C(O)NR₁₀R₁₁, and wherein R₁₀ and R₁₁, together with the nitrogen to which they are both attached, form a 5-6 membered heterocycle which is optionally substituted by aryl or aralkyl.

8. A compound of Formula 4: 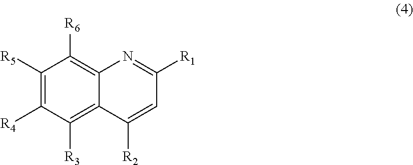
or a pharmaceutically acceptable salt, solvate, tautomer, or prodrug thereof, wherein:
R₁ is substituted C₁-C₆ alkyl, optionally substituted C₁-C₆ alkenyl, optionally substituted aryl, optionally substituted heteroaryl, optionally substituted arylalkyl, optionally substituted heteroarylalkyl, optionally substituted arylalkenyl, —C(O)NZ₁R₇, —NHR₈, —YR₉, —NHYR₉, —C(O)NR₁₀R₁₁, or —C═N—NR_{A}R_{B};
wherein Z₁ is H or C₁-C₆ alkyl;
wherein R₇ and R₈ are independently optionally substituted heterocyclylalkyl, optionally substituted heterocyclylalkylalkyl, optionally substituted aryl, optionally substituted arylalkyl, optionally substituted heteroaryl, optionally substituted heteroarylalkyl, optionally substituted heterocyclylalkynyl, optionally substituted heterocyclylalkenyl, optionally substituted arylalkenyl, optionally substituted arylalkynyl, optionally substituted cycloalkyl, optionally substituted cycloalkylalkyl, optionally substituted alkyl, or H;
wherein R₉ is optionally substituted arylalkyl or optionally substituted heteroarylalkyl;
wherein R₁₀ and R₁₁, together with the nitrogen to which they are both attached, form a heterocycle which is optionally substituted;
wherein Y is C(O) or SO₂; and
wherein R_{A} and R_{B} are each independently C₁-C₆ alkyl;
R₂ is H, hydroxyl, cyano, sulfonamide, sulfone, optionally substituted C₁-C₆ alkyl, optionally substituted C₂-C₆ alkene, optionally substituted C₂-C₆ alkyne, optionally substituted C₁-C₆ alkoxy, optionally substituted aryl, optionally substituted heteroaryl, optionally substituted cycloalkyl, optionally substituted heterocycle, NHR₁₂, NR₁₃R₁₄, S(O)₀₋₂R₁₅, or wherein R₁₂, R₁₃ and R₁₄ are each independently H, sulfonyl, sulfone, sulfonamide, acetyl, optionally substituted C₁-C₆ alkyl, optionally substituted C₁-C₆ alkenyl, optionally substituted C₁-C₆ alkoxy, or hydroxy; and
wherein R₁₅ is sulfonyl, sulfone, sulfonamide, acetyl, optionally substituted C₁-C₆ alkyl, optionally substituted C₁-C₆ alkenyl, optionally substituted C₁-C₆ alkoxy, or hydroxy;
R₃ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide;
R₄ is optionally substituted aryl, optionally substituted heteroaryl, provided that said heteroaryl is not a bicyclic heteroaryl, —C(O)NR₁₆R₁₇, optionally substituted cycloalkyl, optionally substituted cycloalkoxy, optionally substituted C₁-C₆ alkoxy, —NR₁₈R₁₉, optionally substituted C₁-C₆ alkyl, optionally substituted C₂-C₆ alkenyl, optionally substituted C₂-C₆ alkynyl, cyano, —C(O)R₂OR₂₁, —NR₂₂C(O)R₂₃, —NR₂₂S(O)₂R₂₃, or optionally substituted heterocycloalkyl;
wherein each of R₁₆, R₁₇, R₁₈, R₁₉, R₂₀, R₂₁, R₂₂ and R₂₃ are independently H, optionally substituted C₁-C₆ alkyl, or optionally substituted C₂-C₆ alkenyl;
R₅ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide;
R₆ is H, halogen, haloalkyl, C₁-C₆ haloalkoxy, optionally substituted C₁-C₆ alkyl, optionally substituted C₂-C₆ alkenyl, optionally substituted C₂-C₆ alkynyl, optionally substituted aryl, optionally substituted arylalkyl, optionally substituted arylalkenyl, optionally substituted heteroaryl, optionally substituted heteroarylalkyl, optionally substituted heteroarylalkenyl, or —C(O)OH.

9. The compound of claim 8 wherein R₃ is H.

10. The compound of claim 8 wherein R₅ is H or halogen.

11. The compound of claim 8, wherein R₁ is —C(O)NR₁₀R₁₁, and wherein R₁₀ and R₁₁, together with the nitrogen to which they are both attached, form a heterocycle which is optionally substituted.

12. A compound of Formula (5): 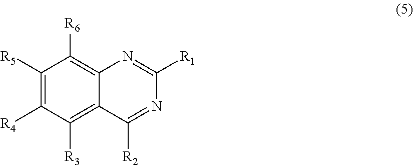
or a pharmaceutically acceptable salt, solvate, tautomer, or prodrug thereof, wherein:
R₁ is substituted C₁-C₆ alkyl, optionally substituted C₁-C₆ alkenyl, optionally substituted aryl, optionally substituted heteroaryl, optionally substituted arylalkyl, optionally substituted heteroarylalkyl, optionally substituted arylalkenyl, —C(O)NZ₁R₇, —NHR₈, —YR₉, —NHYR₉, —C(O)NR₁₀R₁₁, —C═N—NR_{A}R_{B}, or —C═N—O;
wherein Z₁ is H or C₁-C₆ alkyl;
wherein R₇ and R₈ are independently optionally substituted heterocyclylalkyl, optionally substituted heterocyclylalkylalkyl, optionally substituted aryl, optionally substituted arylalkyl, optionally substituted heteroaryl, optionally substituted heteroarylalkyl, optionally substituted heterocyclylalkynyl, optionally substituted heterocyclylalkenyl, optionally substituted arylalkenyl, optionally substituted arylalkynyl, optionally substituted cycloalkyl, optionally substituted cycloalkylalkyl, optionally substituted alkyl, or H;
wherein R₉ is optionally substituted arylalkyl or optionally substituted heteroarylalkyl;
wherein R₁₀ and R₁₁, together with the nitrogen to which they are both attached, form a heterocycle which is optionally substituted;
wherein Y is C(O) or SO₂; and
wherein R_{A} and R_{B} are each independently C₁-C₆ alkyl;
R₂ is H, hydroxyl, cyano, sulfonamide, sulfone, optionally substituted C₁-C₆ alkyl, optionally substituted C₂-C₆ alkene, optionally substituted C₂-C₆ alkyne, optionally substituted C₁-C₆ alkoxy, optionally substituted aryl, optionally substituted heteroaryl, optionally substituted cycloalkyl, optionally substituted heterocycle, NHR₁₂, NR₁₃R₁₄, S(O)₀₋₂R₁₅, or halogen;
wherein R₁₂, R₁₃ and R₁₄ are each independently H, sulfonyl, sulfone, sulfonamide, acetyl, optionally substituted C₁-C₆ alkyl, optionally substituted C₁-C₆ alkenyl, optionally substituted C₁-C₆ alkoxy, or hydroxy; and
wherein R₁₅ is sulfonyl, sulfone, sulfonamide, acetyl, optionally substituted C₁-C₆ alkyl, optionally substituted C₁-C₆ alkenyl, optionally substituted C₁-C₆ alkoxy, or hydroxy;
R₃ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide;
R₄ is optionally substituted aryl, optionally substituted heteroaryl, provided that said heteroaryl is not a bicyclic heteroaryl, —C(O)NR₁₆R₁₇, optionally substituted cycloalkyl, optionally substituted cycloalkoxy, optionally substituted C₁-C₆ alkoxy, —NR₁₈R₁₉, optionally substituted C₁-C₆ alkyl, optionally substituted C₂-C₆ alkenyl, optionally substituted C₂-C₆ alkynyl, cyano, —C(O)R₂OR₂₁, —NR₂₂C(O)R₂₃, —NR₂₂S(O)₂R₂₃, or optionally substituted heterocycloalkyl;
wherein each of R₁₆, R₁₇, R₁₈, R₁₉, R₂₀, R₂₁, R₂₂ and R₂₃ are independently H, optionally substituted C₁-C₆ alkyl, or optionally substituted C₂-C₆ alkenyl;
R₅ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide; and
R₆ is H, halogen, haloalkyl, C₁-C₆ haloalkoxy, optionally substituted C₁-C₆ alkyl, optionally substituted C₂-C₆ alkenyl, optionally substituted C₂-C₆ alkynyl, optionally substituted aryl, optionally substituted arylalkyl, optionally substituted arylalkenyl, optionally substituted heteroaryl, optionally substituted heteroarylalkyl, optionally substituted heteroarylalkenyl, or —C(O)OH.

13. The compound of claim 12 wherein R₃ is H.

14. The compound of claim 12 wherein R₅ is H or halogen.

15. The compound of claim 12, wherein R₁ is —C(O)NR₁₀R₁₁, and wherein R₁₀ and R₁₁, together with the nitrogen to which they are both attached, form a heterocycle which is optionally substituted.

16. A compound of Formula 6: 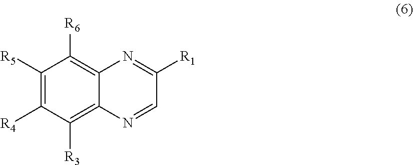
or a pharmaceutically acceptable salt, solvate, tautomer, or prodrug thereof, wherein:
R₁ is optionally substituted C₁-C₆ alkyl, optionally substituted C₁-C₆ alkenyl, optionally substituted aryl, optionally substituted heteroaryl, optionally substituted arylalkyl, optionally substituted heteroarylalkyl, optionally substituted arylalkenyl, —C(O)NZ₁R₇, —NHR₈, —YR₉, —NHYR₉, —C(O)NR₁₀R₁₁, —C═N—NR_{A}R_{B}, or —C═N—O;
wherein Z₁ is H or C₁-C₆ alkyl;
wherein R₇ and R₈ are independently optionally substituted heterocyclylalkyl, optionally substituted heterocyclylalkylalkyl, optionally substituted aryl, optionally substituted arylalkyl, optionally substituted heteroaryl, optionally substituted heteroarylalkyl, optionally substituted heterocyclylalkynyl, optionally substituted heterocyclylalkenyl, optionally substituted arylalkenyl, optionally substituted arylalkynyl, optionally substituted cycloalkyl, optionally substituted cycloalkylalkyl, optionally substituted alkyl, or H;
wherein R₉ is optionally substituted arylalkyl or optionally substituted heteroarylalkyl;
wherein R₁₀ and R₁₁, together with the nitrogen to which they are both attached, form a heterocycle which is optionally substituted;
wherein Y is C(O) or SO₂; and
wherein R_{A} and R_{B} are each independently C₁-C₆ alkyl;
R₃ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide;
R₄ is optionally substituted aryl, optionally substituted heteroaryl, provided that said heteroaryl is not a bicyclic heteroaryl, —C(O)NR₁₆R₁₇, optionally substituted cycloalkyl, optionally substituted cycloalkoxy, optionally substituted C₁-C₆ alkoxy, —NR₁₈R₁₉, optionally substituted C₁-C₆ alkyl, optionally substituted C₂-C₆ alkenyl, optionally substituted C₂-C₆ alkynyl, cyano, —C(O)R₂₀R₂₁, —NR₂₂C(O)R₂₃, —NR₂₂S(O)₂R₂₃, or optionally substituted heterocycloalkyl;
wherein each of R₁₆, R₁₇, R₁₈, R₁₉, R₂₀, R₂₁, R₂₂ and R₂₃ are independently H, optionally substituted C₁-C₆ alkyl, or optionally substituted C₂-C₆ alkenyl;
R₅ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide; and
R₆ is H, halogen, haloalkyl, C₁-C₆ haloalkoxy, optionally substituted C₁-C₆ alkyl, optionally substituted C₂-C₆ alkenyl, optionally substituted C₂-C₆ alkynyl, optionally substituted aryl, optionally substituted arylalkyl, optionally substituted arylalkenyl, optionally substituted heteroaryl, optionally substituted heteroarylalkyl, optionally substituted heteroarylalkenyl, or —C(O)OH.

17. The compound of claim 16 wherein R₃ is H.

18. The compound of claim 16 wherein R₅ is H or halogen.

19. The compound of claim 18 wherein R₃ is H.

20. The compound of claim 16, wherein R₁ is —C(O)NR₁₀R₁₁, and wherein R₁₀ and R₁₁, together with the nitrogen to which they are both attached, form a heterocycle which is optionally substituted.

21. A compound of Formula (7): 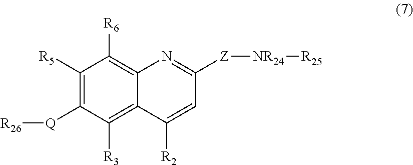
or a pharmaceutically acceptable salt, solvate, tautomer, or prodrug thereof, wherein:
R₂ is H, hydroxyl, cyano, sulfonamide, sulfone, optionally substituted C₁-C₆ alkyl, optionally substituted C₂-C₆ alkene, optionally substituted C₂-C₆ alkyne, optionally substituted C₁-C₆ alkoxy, optionally substituted aryl, optionally substituted heteroaryl, optionally substituted cycloalkyl, optionally substituted heterocycle, NHR₁₂, NR₁₃R₁₄, S(O)₀₋₂R₁₅, or halogen;
R₃ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide;
R₅ is H or halo;
R₆ is H, halogen, haloalkyl, (C₁-C₆) haloalkoxy, optionally substituted (C₁-C₆) alkyl, optionally substituted (C₂-C₆) alkenyl, optionally substituted (C₂-C₆) alkynyl, optionally substituted aryl, optionally substituted arylalkyl, optionally substituted arylalkenyl, optionally substituted heteroaryl, optionally substituted heteroarylalkyl, optionally substituted heteroarylalkenyl, or —C(O)OH;
Z is carbocyclyl, aryl, O-carbocyclyl, O-aryl, or a 5-6 membered heterocyclyl;
Q is CH₂, O, N or S;
R₂₄ is H or optionally substituted (C₁-C₆)alkyl;
R₂₅ is H, (C₁-C₆)alkyl, (C₂-C₆)alkynyl, (C₁-C₆)haloalkyl, (C₂-C₆)haloalkenyl, (C₂-C₆)haloalkynyl, carbocyclyl, aryl, or heterocyclyl and
R₂₆ is H, (C₁-C₆)alkyl, (C₂-C₆)alkenyl, (C₂-C₆)alkynyl, (C₁-C₆)haloalkyl, (C₂-C₆)haloalkenyl, (C₂-C₆)haloalkynyl, carbocyclyl, aryl, or heterocyclyl.

22. The compound of claim 21 wherein R₃ is H.

23. The compound of claim 21 wherein R₅ is H or F.

24. The compound of claim 21 wherein R₆ is (C₁-C₆)alkyl, (C₂-C₆)alkenyl, (C₂-C₆)alkynyl, (C₁-C₃)haloalkyl, or O—(C₁-C₃)haloalkyl.

25. A compound of Formula (8): 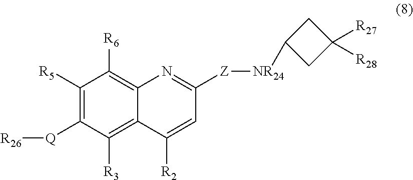
or a pharmaceutically acceptable salt, solvate, tautomer, or prodrug thereof, wherein:
R₂ is H, hydroxyl, cyano, sulfonamide, sulfone, optionally substituted C₁-C₆ alkyl, optionally substituted C₂-C₆ alkene, optionally substituted C₂-C₆ alkyne, optionally substituted C₁-C₆ alkoxy, optionally substituted aryl, optionally substituted heteroaryl, optionally substituted cycloalkyl, optionally substituted heterocycle, NHR₁₂, NR₁₃R₁₄, S(O)₀₋₂R₁₅, or halogen;
R₃ is H, halogen, optionally substituted C₁-C₆ alkyl, optionally substituted C₂-C₆ alkenyl, C₁-C₆ haloalkyl, cyano, optionally substituted C₁-C₆ alkoxy, sulfone, or sulfonamide;
R₅ is H or F;
R₆ is H, halogen, haloalkyl, (C₁-C₆) haloalkoxy, optionally substituted (C₁-C₆) alkyl, optionally substituted (C₂-C₆) alkenyl, optionally substituted (C₂-C₆) alkynyl, optionally substituted aryl, optionally substituted arylalkyl, optionally substituted arylalkenyl, optionally substituted heteroaryl, optionally substituted heteroarylalkyl, optionally substituted heteroarylalkenyl, and —C(O)OH;
Z is a 5-6 membered heterocyclyl;
Q is CH₂, O or S;
R₂₄ is H or (C₁-C₃)alkyl
R₂₆ is H, (C₁-C₆)alkyl, (C₂-C₆)alkenyl, (C₂-C₆)alkynyl, (C₁-C₆)haloalkyl, (C₂-C₆)haloalkenyl, (C₂-C₆)haloalkynyl, carbocyclyl, aryl, or heterocyclyl.
R₂₇ is H, O, N, S, hydroxyl, phosphate, or optionally substituted (C₁-C₆)alkyl; and
R₂₈ is H, (C₁-C₆)alkyl, (C₂-C₆)alkenyl, (C₂-C₆)alkynyl, (C₁-C₆)haloalkyl, (C₂-C₆)haloalkenyl, or (C₂-C₆)haloalkynyl.

26. The compound of claim 25 wherein Z is oxadiazolyl or thiadiazolyl.

27. The compound of claim 25 wherein R₂₄ is H.

28. The compound of claim 25 wherein R₂₇ is hydroxyl.

29. The compound of claim 25 wherein R₂₈ is (C₁-C₆)haloalkyl.

30. The compound of claim 25 wherein:
R₂ is methyl;
R₃ is H
Z is oxadiazolyl or thiadiazolyl;
Q is O or S;
R₅ is H;
R₆ is (C₁-C₆)alkyl, (C₁-C₃)haloalkyl, or O—(C₁-C₃)haloalkyl;
R₂₆ is (C₁-C₆)alkyl or (C₁-C₆)haloalkyl.
R₂₇ is H, OH, phosphate, or optionally substituted (C₁-C₆)alkyl; and
R₂₈ is (C₁-C₆)alkyl, or (C₁-C₆)haloalkyl.

31. A compound selected from the group consisting of:

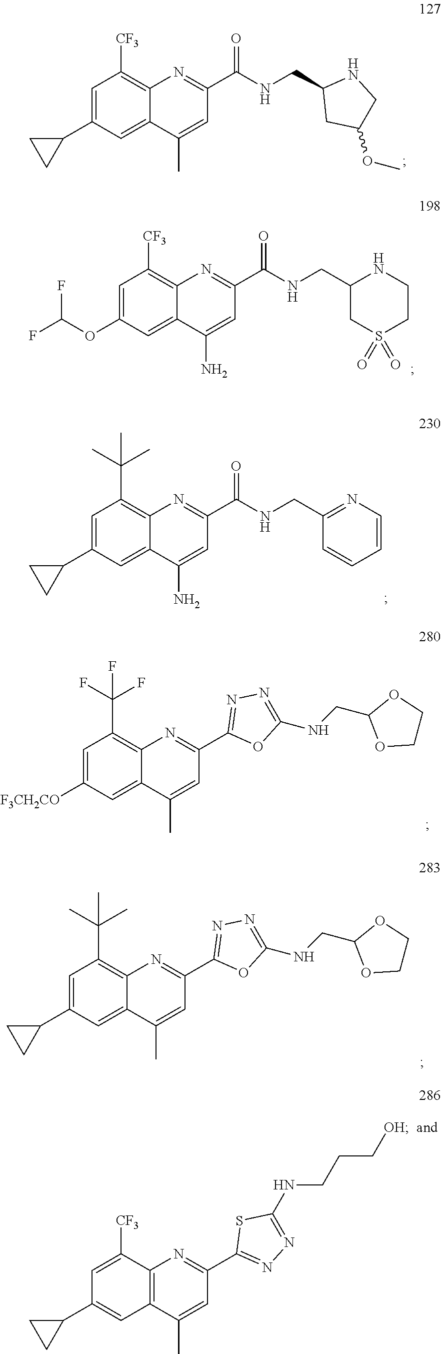

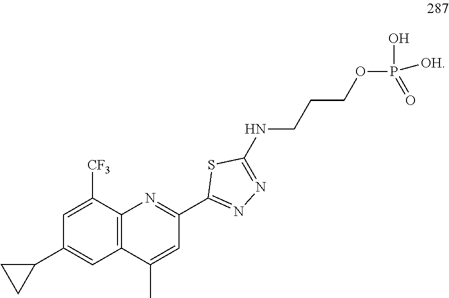

32. A compound selected from the group consisting of: 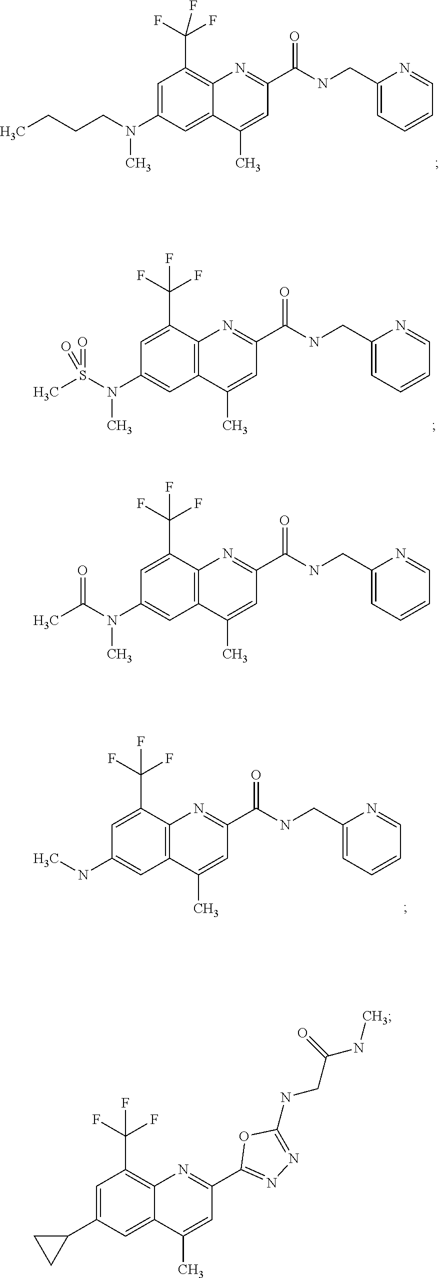 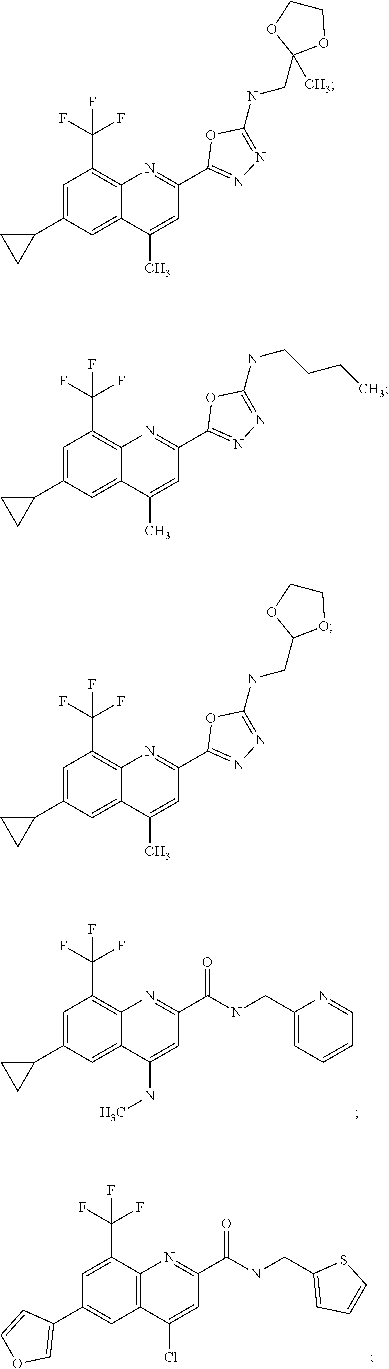 [chemical structure]
